(19) United States
(12) Patent Application Publication
(10) Pub. No.: US 2022/0064255 A1
(43) Pub. Date: Mar. 3, 2022

(54) ANTI-TCR ANTIBODY MOLECULES AND USES THEREOF

(71) Applicant: Marengo Therapeutics, Inc., Cambridge, MA (US)

(72) Inventors: Andreas Loew, Boston, MA (US); Stephen Arkinstall, Belmont, MA (US)

(21) Appl. No.: 17/366,638

(22) Filed: Jul. 2, 2021

Related U.S. Application Data
(63) Continuation of application No. PCT/US2020/012162, filed on Jan. 3, 2020.
(60) Provisional application No. 62/788,497, filed on Jan. 4, 2019.
(60) Provisional application No. 62/803,893, filed on Feb. 11, 2019.

(51) Int. Cl. C07K 14/725; C12N 5/0783; C07K 16/28; C07K 14/705

(52) U.S. Cl. CPC C07K 14/7051; C12N 5/0636; C07K 16/2809; C07K 16/2803; C07K 14/70521; C07K 2317/76; C07K 2317/622; C07K 2319/30; C07K 2319/33; C07K 2317/77; C07K 2317/33; C12N 2501/515

(57) ABSTRACT

The disclosure provides methods of expanding T cells ex vivo comprising contacting the T cells with antibody molecules that bind to TCR Vβ regions. In some embodiments, the T cells comprise one or more nucleic acid molecule encoding an exogenous cellular receptor, for example, a chimeric antigen receptor (CAR) or an exogenous T cell receptor (TCR).

### RELATED APPLICATIONS

This application is a continuation of International Application No. PCT/US2020/012162, filed on Jan. 3, 2020, which claims the benefit of U.S. Provisional Patent Application No. 62/788,497, filed on Jan. 4, 2019, and U.S. Provisional Patent Application No. 62/803,893, filed on Feb. 11, 2019; the disclosures of each of which are hereby incorporated by reference herein in their entirety.

### SEQUENCE LISTING

The instant application contains a Sequence Listing which has been submitted electronically in ASCII format and is hereby incorporated by reference in its entirety. Said ASCII copy, created on Jan. 31, 2020, is named 53676-702_301_SL.txt and is 311,084 bytes in size.

### BACKGROUND

Current molecules designed to activate and expand T cells encoding exogenous receptors (e.g., CAR T cells, T cells expressing an exogenous TCR) ex vivo for cancer immunotherapy typically target the CD3 epsilon (CD3ε) subunit of the T cell receptor (TCR) alone or in combination with targeting the costimulatory receptor CD28. However, there are limitations to this approach. Previous studies have shown that use of these anti-CD3ε targeting molecules can produce T cells that when infused into a subject either produce or stimulate other cells to produce proinflammatory cytokines (e.g., IL-1, IL-6 and TNFα) associated with inflammatory conditions such cytokine release syndrome (CRS), macrophage activation syndrome, neurological toxicities, and tumor lysis syndrome. Thus, a current need exists to develop additional methods of activating and expanding T cells ex vivo, which do not pose these significant risks to patients.

### SUMMARY

This disclosure is based, at least in part, on the unexpected discovery that T cells can be activated and expanded ex vivo using anti-TCRVβ antibodies; and that these T cells secrete substantially lower levels of proinflammatory cytokines associated with the induction of cytokine release syndrome (CRS), macrophage activation syndrome, neurological toxicities, and tumor lysis syndrome, such as IFNγ, IL-10, IL-17A, IL-1α, IL-1β, IL-2, IL-6, and TNFα in vivo; while also secreting higher or similar levels of IL-2.

Disclosed herein are, inter alio, methods of expanding T cells ex vivo using antibodies directed to the variable chain of the beta subunit of TCR (TCRβV). In some embodiments, methods described herein result in less or no production of cytokines associated with cytokine release syndrome (CRS), e.g., IL-6, IL-1beta and TNF alpha; and enhanced and/or delayed production of IL-2 and IFNγ. In some embodiments, methods described herein limit the unwanted side-effects of CRS, e.g., CRS associated with anti-CD3e targeting.

According, in one aspect, provided herein are methods of expanding T cells ex vivo comprising contacting a plurality of T cells to a first agent, wherein the first agent comprises a first domain that specifically binds to a T cell receptor beta variable beta chain (TCRβV) region, thereby generating a first population of T cells.

In some embodiments, the first agent further comprises a second domain that binds to a protein expressed on the surface of a population of T cells in the plurality.

In some embodiments, the first agent is a bispecific antibody molecule.

In some embodiments, the second domain specifically binds to a T cell receptor variable beta chain (TCRβV) region.

In some embodiments, the second domain and the first domain specifically bind to different T cell receptor variable beta chain (TCRβV) regions.

In some embodiments, the second domain and the first domain specifically bind to TCRβVs belonging to different subfamilies or different members of the same TCRβV subfamily.

In some embodiments, the first domain specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily, and the second domain specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily.

In some embodiments, the first domain specifically binds to a TCRβV region of a TCRβV belonging to a TCRβV12 subfamily.

In some embodiments, the second domain and the first domain specifically bind to TCRβVs belonging to different subfamilies.

In some embodiments, the second domain and the first domain specifically bind to different members of the same TCRβV subfamily.

In some embodiments, the second domain specifically binds to an antibody molecule. In some embodiments, the antibody molecule is expressed by a population of T cells in the plurality. In some embodiments, the antibody molecule comprises a variable heavy chain and a variable light chain. In some embodiments, the antibody molecule is a scFv or a Fab.

In some embodiments, the second domain specifically binds to a light chain region of the antibody molecule. In some embodiments, the second domain specifically binds to a κ light chain region of an antibody molecule. In some embodiments, the second domain comprises protein L.

In some embodiments, the first domain comprises LC CDR1, LC CDR2, LC CDR, HC CDR1, HC CDR 2, and HC CDR 3 of an antibody described in Table 2, Table 3, Table 4 or Table 5. In some embodiments, the first domain comprises a VH and VL chain sequences of an antibody disclosed in Table 2, Table 3, Table 4, or Table 5.

In some embodiments, the first agent comprises LC CDR1, LC CDR2, LC CDR, HC CDR1, HC CDR 2, and HC CDR 3 of an antibody described in Table 2, Table 3, Table 4 or Table 5. In some embodiments, the first agent comprises a VH and VL chain sequences of an antibody disclosed in Table 2, Table 3, Table 4, or Table 5.

In some embodiments, said first agent specifically binds to at least two TCRβVs belonging to different subfamilies.

In some embodiments, said first agent specifically binds to at least three, four, five, or six TCRβVs belonging to different subfamilies.

In some embodiments, said first agent specifically binds to at least two different members of the same TCRβV subfamily.

In some embodiments, said first agent specifically binds to at least three, four, five, six, or seven different members of the same TCRβV subfamily.

In some embodiments, the method further comprises contacting the plurality of T cells with a second agent, wherein the second agent comprises a domain that specifically binds to a T cell receptor variable beta chain (TCRβV) region, wherein the first and the second agents specifically bind to different TCRβV regions.

In some embodiments, the first agent comprises a domain that specifically binds to a TCRβV region of a first TCRβV, and the second agent comprises a domain that specifically binds to a TCRβV region of a second TCRβV, wherein the first and the second TCRβVs belong to different TCRβV subfamilies or are different members of the same TCRβV subfamily.

In some embodiments, the first domain specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily, and the second domain specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily.

In some embodiments, the first agent comprises a domain that specifically binds to a TCRβV region of a first TCRβV belonging to a TCRβV12 subfamily.

In some embodiments, the first and the second agent each specifically bind to a TCRβV belonging to a different subfamily.

In some embodiments, the first and the second agent each specifically bind to different members of the same TCRβV subfamily.

In some embodiments, the first population of T cells exhibit at least one (e.g., at least 2, 3, 4, 5, 6, 7, or 8) of: a lower level of IL-1β expression, a lower level of IL-6 expression, a lower level of TNFα expression, a lower level of IFNγ expression, a lower level of IL-1β expression, a lower level of IL-17 expression, a higher level of IL-2 expression, or a higher level of IL-15 expression, relative to a comparable population of T cells that contacted with an agent comprising a domain that specifically binds CD3ε (e.g., an anti-CD3ε antibody).

In some embodiments, expression is measured by determining the level of the protein secreted from the population of T cells, as measured by an assay described herein.

In some embodiments, the level of IL-1β expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-6 expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-10 expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-17 expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IFN-γ expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of TNF-α expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-15 expression is at least 5%, 10%, 15%, 20%, 25%, 30%, 35%, 40%, 45%, 50%, 55%, 60%, 65%, 70% or 75% higher than the level expressed by the comparable population of T cells contacted with an agent comprising a domain that specifically binds CD3ε, as measured by an assay described herein.

In some embodiments, the level of IL-2 expression is at least 5%, 10%, 15%, 20%, 25%, 30%, 35%, 40%, 45%, 50%, 55%, 60%, 65%, 70% or 75% greater than the level expressed by the comparable population of T cells contacted with an agent comprising a domain that specifically binds CD3ε, as measured by an assay described herein.

In some embodiments, the number of T cells in the first population of T cells, it at least about 10 fold higher (e.g., at least 50, 100, 500, 1000, or 10000 fold higher) than the number of T cells in the plurality of T cells.

In some embodiments, the number of T cells in the first population of T cells that express CD45R, express CD95, and exhibit low or no detectable expression of CCR7 is higher compared to the number of T cells in a comparable population that express CD45R, express CD95, and exhibit low or no detectable expression of CCR7 contacted with an agent comprising a domain that specifically binds CD3ε (e.g., an anti-CD3ε antibody).

In some embodiments, the number of T cells in the first population that express CD45R, express CD95, and exhibit low or no detectable expression of CCR7 is at least 2, 3, 4, 5, 10, 15, 20, 50, 100, 500, or 1000 fold higher than the number of T cells in in a comparable population that express CD45R, express CD95, and exhibit low or no detectable expression of CCR7 contacted with an agent comprising a domain that specifically binds CD3ε (e.g., an anti-CD3 antibody).

In some embodiments, the expression of CD45R, CD95, and CCR7 is measured by determining the level of the protein on the surface of the cell (e.g., as measured by flow cytometry).

In some embodiments, the number of TEMRA T cells in the first population is higher than the number of TEMRA T cells in a comparable population of T cells contacted with an agent comprising a domain that specifically binds CD3ε (e.g., an anti-CD3ε antibody).

In some embodiments, the number of TEMRA T cells in the first population is at least 2, 3, 4, 5, 10, 15, 20, 50, 100, 500, or 1000 fold higher than the number of TEMRA T cells in a comparable population of T cells contacted with an agent comprising a domain that specifically binds CD3ε (e.g., an anti-CD3ε antibody).

In some embodiments, the contacting comprises incubating the plurality of T cells with the first agent.

In some embodiments, contacting comprises incubating or culturing the plurality of T cells with the first agent for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days.

In some embodiments, contacting comprises incubating or culturing the plurality of T cells with the first agent for at most about 10 minutes, 20 minutes, 30 minutes, 1 hour, 6 hours, 10 hours, 12 hours, 24 hours, 36 hours, 48 hours, 72 hours, 5 days, 7 days, 10 days, 12 days, 14 days, 15 days, 21 days, 30 days, 45 days, or 60 days.

In some embodiments, contacting comprises incubating or culturing the plurality of T cells with the first agent for from about 10-90 minutes, 10-60 minutes, 10-30 minutes, 1-30 days, 1-21 days, 1-14 days, 1-7 days, 1-5 days, 1-3 days, 21-30 days, 14-30 days, 7-30 days, 5-30 days, or 3-30 days.

In some embodiments, the first agent is coupled to a solid surface (e.g., a bead, a cell culture plate). In some embodiments, said coupling enables cross linking of the TCRs on the surface of the plurality of T cells specifically bound by the first agent.

In some embodiments, the first agent comprises an antibody domain. In some embodiments, the first agent comprises an anti-idiotypic antibody domain. In some embodiments, the first agent comprises a human or humanized antibody domain. In some embodiments, the first agent comprises an antigen binding domain comprising a single chain Fv (scFv) or a Fab. In some embodiments, the first agent comprises an antibody comprising two antibody heavy chains, each of the two heavy chains comprising a variable region and a constant region; and two antibody light chains, each of the two light chains comprising a variable region and a constant region.

In some embodiments, the plurality of T cells comprises a population of T cells that comprise an exogenous nucleic acid. In some embodiments, the exogenous nucleic acid encodes a cell surface receptor. In some embodiments, the cell surface receptor is a chimeric antigen receptor (CAR) or a T cell receptor (TCR). In some embodiments, the method further comprises introducing an exogenous nucleic acid into at least a portion of T cells of the plurality prior to contacting the plurality of T cells with the first agent. In some embodiments, the method further comprises introducing an exogenous nucleic acid into at least a portion of T cells of the plurality after contacting the plurality of T cells with the first agent. In some embodiments, the exogenous nucleic acid is introduced by transduction or transfection.

In some embodiments, the plurality of T cells are human. In some embodiments, the plurality of T cells comprises T cells from a human subject that was healthy when the cells were removed (e.g., a subject that does not have or has not been diagnosed with a predetermined disease or condition, e.g., a cancer). In some embodiments, the plurality of T cells comprises T cells from a human subject having or diagnosed with a disease or condition when the cells were removed (e.g., diagnosed with a predetermined disease or condition, e.g., cancer). In some embodiments, the disease is a cancer.

In one aspect, provided herein are methods of expanding T cells ex vivo comprising contacting a plurality of T cells to a plurality of agents, wherein the plurality of agents comprises at least a first and a second agent, wherein each agent of the plurality comprises a domain that specifically binds to a different T cell receptor variable beta chain (TCRβV) region, thereby generating a first population of T cells.

In some embodiments, said first agent or said second agent or both specifically binds to at least two TCRβVs belonging to different subfamilies.

In some embodiments, said first agent or said second agent or both specifically binds to at least three, four, five, or six TCRβVs belonging to different subfamilies.

In some embodiments, said first agent or said second agent or both specifically binds to at least two different members of the same TCRβV subfamily.

In some embodiments, said first agent or said second agent or both specifically binds to at least three, four, five, six, or seven different members of the same TCRβV subfamily.

In some embodiments, the plurality comprises at least three, four, five, six, seven, eight, nine, or ten agents, wherein each agent of the plurality comprises a domain that specifically binds to a different T cell receptor variable beta chain (TCRβV) region.

In some embodiments, each agent of the plurality specifically binds to a different TCRβV, wherein each TCRβV belongs to a different TCRβV subfamily or are different members of the same TCRβV subfamily.

In some embodiments, each agent of the plurality comprises a domain that specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily.

In some embodiments, at least one agent of said plurality comprises a domain that specifically binds to a TCRβV region of a TCRβV belonging to a TCRβV12 subfamily.

In some embodiments, each agent of the plurality specifically binds to a different TCRβV, wherein each TCRβV belongs to a different TCRβV subfamily.

In some embodiments, each agent of the plurality specifically binds to a different TCRβV, wherein each TCRβV or are different members of the same TCRβV subfamily.

In some embodiments, the first population of T cells exhibit at least one (e.g., at least 2, 3, 4, 5, 6, 7, or 8) of: a lower level of IL-1β expression, a lower level of IL-6 expression, a lower level of TNFα expression, a lower level of IFNγ expression, a lower level of IL-10 expression, a lower level of IL-17 expression, a higher level of IL-2 expression, or a higher level of IL-15 expression, relative to a comparable population of T cells that contacted with an agent comprising a domain that specifically binds CD3ε (e.g., an anti-CD3ε antibody).

In some embodiments, expression is measured by determining the level of the protein secreted from the population of T cells, as measured by an assay described herein.

In some embodiments, the level of IL-1β expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-6 expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-1β expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-17 expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IFN-γ expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of TNF-α expression is at least 5%, 10%, 15%, 20%, 25%, 30%, 35%, 40%, 45%, 50%, 55%, 60%, 65%, 70% or 75% less than the level expressed by the comparable population of T cells contacted with an agent comprising a domain that specifically binds CD3ε, as measured by an assay described herein.

In some embodiments, the level of IL-15 expression is at least 5%, 10%, 15%, 20%, 25%, 30%, 35%, 40%, 45%, 50%, 55%, 60%, 65%, 70% or 75% higher than the level expressed by the comparable population of T cells contacted with an agent comprising a domain that specifically binds CD3ε, as measured by an assay described herein.

In some embodiments, the level of IL-2 expression is at least 5%, 10%, 15%, 20%, 25%, 30%, 35%, 40%, 45%, 50%, 55%, 60%, 65%, 70% or 75% greater than the level expressed by the comparable population of T cells contacted with an agent comprising a domain that specifically binds CD3ε, as measured by an assay described herein.

In some embodiments, the number of T cells in the first population of T cells, it at least about 10 fold higher (e.g., at least 50, 100, 500, 1000, or 10000 fold higher) than the number of T cells in the plurality of T cells.

In some embodiments, the number of T cells in the first population of T cells that express CD45R, express CD95, and exhibit low or no detectable expression of CCR7 is higher compared to the number of T cells in a comparable population that express CD45R, express CD95, and exhibit low or no detectable expression of CCR7 contacted with an agent comprising a domain that specifically binds CD3ε (e.g., an anti-CD3ε antibody).

In some embodiments, the number of T cells in the first population that express CD45R, express CD95, and exhibit low or no detectable expression of CCR7 is at least 2, 3, 4, 5, 10, 15, 20, 50, 100, 500, or 1000 fold higher than the number of T cells in in a comparable population that express CD45R, express CD95, and exhibit low or no detectable expression of CCR7 contacted with an agent comprising a domain that specifically binds CD3ε (e.g., an anti-CD3 antibody).

In some embodiments, the expression of CD45R, CD95, and CCR7 is measured by determining the level of the protein on the surface of the cell (e.g., as measured by flow cytometry).

In some embodiments, the number of TEMRA T cells in the first population is higher than the number of TEMRA T cells in a comparable population of T cells contacted with an agent comprising a domain that specifically binds CD3ε (e.g., an anti-CD3ε antibody).

In some embodiments, the number of TEMRA T cells in the first population is at least 2, 3, 4, 5, 10, 15, 20, 50, 100, 500, or 1000 fold higher than the number of TEMRA T cells in a comparable population of T cells contacted with an agent comprising a domain that specifically binds CD3ε (e.g., an anti-CD3ε antibody).

In some embodiments, the contacting comprises incubating the plurality of T cells with the first agent.

In some embodiments, contacting comprises incubating or culturing the plurality of T cells with the first agent for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days.

In some embodiments, contacting comprises incubating or culturing the plurality of T cells with the first agent for at most about 10 minutes, 20 minutes, 30 minutes, 1 hour, 6 hours, 10 hours, 12 hours, 24 hours, 36 hours, 48 hours, 72 hours, 5 days, 7 days, 10 days, 12 days, 14 days, 15 days, 21 days, 30 days, 45 days, or 60 days.

In some embodiments, contacting comprises incubating or culturing the plurality of T cells with the first agent for from about 10-90 minutes, 10-60 minutes, 10-30 minutes, 1-30 days, 1-21 days, 1-14 days, 1-7 days, 1-5 days, 1-3 days, 21-30 days, 14-30 days, 7-30 days, 5-30 days, or 3-30 days.

In some embodiments, the first agent is coupled to a solid surface (e.g., a bead, a cell culture plate). In some embodiments, said coupling enables cross linking of the TCRs on the surface of the plurality of T cells specifically bound by the first agent.

In some embodiments, the first agent comprises an antibody domain. In some embodiments, the first agent comprises an anti-idiotypic antibody domain. In some embodiments, the first agent comprises a human or humanized antibody domain. In some embodiments, the first agent comprises an antigen binding domain comprising a single chain Fv (scFv) or a Fab. In some embodiments, the first agent comprises an antibody comprising two antibody heavy chains, each of the two heavy chains comprising a variable region and a constant region; and two antibody light chains, each of the two light chains comprising a variable region and a constant region.

In some embodiments, the plurality of T cells comprises a population of T cells that comprise an exogenous nucleic acid. In some embodiments, the exogenous nucleic acid encodes a cell surface receptor. In some embodiments, the cell surface receptor is a chimeric antigen receptor (CAR) or a T cell receptor (TCR). In some embodiments, the method further comprises introducing an exogenous nucleic acid into at least a portion of T cells of the plurality prior to contacting the plurality of T cells with the first agent. In some embodiments, the method further comprises introducing an exogenous nucleic acid into at least a portion of T cells of the plurality after contacting the plurality of T cells with the first agent. In some embodiments, the exogenous nucleic acid is introduced by transduction or transfection.

In some embodiments, the plurality of T cells are human. In some embodiments, the plurality of T cells comprises T cells from a human subject that was healthy when the cells were removed (e.g., a subject that does not have or has not been diagnosed with a predetermined disease or condition, e.g., a cancer). In some embodiments, the plurality of T cells comprises T cells from a human subject having or diagnosed with a disease or condition when the cells were removed (e.g., diagnosed with a predetermined disease or condition, e.g., cancer). In some embodiments, the disease is a cancer.

In one aspect, provided herein are methods of treating cancer in a subject, the method comprising administering at least a portion of the first population of cells described herein or a pharmaceutical composition comprising at least a portion of the first population of cells described herein.

In some embodiments, the plurality of T cells express an exogenous cell surface receptor. In some embodiments, the exogenous cell surface receptor is a chimeric antigen receptor (CAR) or an exogenous T cell receptor (TCR).

In some embodiments, the cell is autologous or allogenic to the subject administered said cell.

In some embodiments, the cancer is a solid cancer or hematological cancer.

The method of any one of claim 81, wherein the cancer is a solid cancer.

In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer.

In some embodiments, the cancer is a hematologic cancer.

In some embodiments, the hematologic cancer is a leukemia, lymphoma, or myeloma.

In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

In one aspect, provided herein are methods of treating cancer in a subject, the method comprising: removing a plurality of T cells from a human subject, expanding at least a portion of the plurality of T cells from the human subject by the method described herein, to thereby generate the first population of T cells, administering at least a portion of the first population of T cells into the human subject, to thereby treat the cancer in the subject.

In some embodiments, the plurality of T cells express an exogenous cell surface receptor. In some embodiments, the exogenous cell surface receptor is a chimeric antigen receptor (CAR) or an exogenous T cell receptor (TCR).

In some embodiments, the cell is autologous or allogenic to the subject administered said cell.

In some embodiments, the cancer is a solid cancer or hematological cancer.

The method of any one of claim 81, wherein the cancer is a solid cancer.

In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer.

In some embodiments, the cancer is a hematologic cancer.

In some embodiments, the hematologic cancer is a leukemia, lymphoma, or myeloma.

In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

In one aspect, provided herein are methods of preventing or lessening cytokine release syndrome (CRS) in a human subject, the method comprising: removing a plurality of T cells from a human subject, expanding at least a portion of the plurality of T cells from the human subject by a method described herein, to thereby generate the first population of T cells, administering at least a portion of the first population of T cells into the human subject, wherein after the administration (e.g., within 24 hours, 48 hours, 72 hours, 4 days, 5 days, 6 days, 7 days, 14 days, 21 days, or 30 days) the subject shows no symptoms of cytokine release syndrome or at least one symptom of CRS is less severe relative to a human subject administered with at least a portion of a comparable population of T cells generated by expanding the T cells by contacting the plurality of T cells with an agent that binds CD3ε (e.g., an anti-CD3ε antibody).

In some embodiments, the at least one symptom is selected from those listed in Table 8, Table 9, or Table 10.

In some embodiments, the at least one symptom is selected from hemophagocytic lymphohistiocytosis (HLH), fever, nausea, vomiting, chills, hypotension, tachycardia, arrhythmia, cardiomyopathy, acute heart failure, asthenia, headache, rash, dyspnea, encephalopathy, aphasia, tremor, ataxia, hemiparesis, palsy, dysmetria, seizure, motor weakness, loss of consciousness, hallucinations, cerebral edema, hepatomegaly, hypofibrinogeniemia, liver failure, diarrhea, edema, rigor, arthralgia, myalgia, acute kidney failure, splenomegaly, respiratory failure, pulmonary edema, hypoxia, capillary leak syndrome, macrophage activation syndrome, or tachypnea.

The method of any one of claims 87-89, wherein the subject does not exhibit at least one symptom of CRS (e.g., as described herein) within 1, 2, 3, 4, 5, 6, 7, 10, 14, 21, or 30 days of administration of the at least a portion of the first population of T cells.

In some embodiments, the subject does not exhibit at least one symptom grade 4 or grade 5 CRS (e.g., as described herein).

In some embodiments, the subject does not exhibit any symptom grade 4 or grade 5 CRS (e.g., as described herein).

In some embodiments, the level of one or more protein selected from the group consisting of IL-6, IL-10, IL-8, IL-10, IFNγ, TNFα, sIL2Rα, sgp130, sIL6R, MCP1, MIP1α, MIP1β, and GM-CSF, in the serum of the subject post administration (e.g., 1 hour, 6 hours, 12 hours, 1 day, 2 days, 3 days, 4 days, 5, days, 6 days, 7 days, 10 days, 14 days, 21 days, 30 days) of the at least a portion of the first population of T cells is within ±20%, ±15%, ±10%, ±9%, ±8%, ±7%, ±6%, ±5%, ±4%, ±3%, ±2% or ±1% of the level of the one or more protein in the serum of the subject prior to administration (e.g., 10 minutes, 30 minutes, 1 hour, 6 hours, 12 hours, 24 hours, 48 hours, 72 hours) of the at least a portion of the first population of T cells.

In some embodiments, the method further comprises selecting the subject for administration of the first population of T cells described herein based on a determination of at least one of the following: the subject's risk of developing CRS, the subject's risk of developing CRS if administered a cell expressing a CAR comprising a CD3ζ signaling domain, the subject's diagnosis of CRS, or the subject's diagnosis of CRS associated with or induced by administration of a cell expressing a CAR comprising a CD3ζ signaling domain.

In some embodiments, the subject is selected for administration if the subject is at risk of developing CRS, if the subject is at risk of developing CRS if administered a CAR comprising a cell expressing a CAR CD3ζ signaling domain, if the subject has been diagnosed with CRS, or if the subject has been diagnosed with CRS associated with or induced by administration of a cell expressing a CAR comprising a CD3ζ signaling domain.

In some embodiments, the cell is autologous or allogenic to the subject administered said cell.

In some embodiments, the cancer is a solid cancer or hematological cancer.

In some embodiments, the cancer is a solid cancer.

In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer.

In some embodiments, the cancer is a hematologic cancer.

In some embodiments, the hematologic cancer is a leukemia, lymphoma, or myeloma.

In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

In one aspect, provided herein are recombinant nucleic acid molecule encoding a chimeric antigen receptor (CAR), wherein the CAR comprises (a) an antigen binding domain, wherein the antigen binding domain does not contain a T cell receptor α (TCRα) variable region or a T cell receptor β (TCRβ) variable region; (b) a transmembrane domain; and (c) an intracellular signaling domain comprising a TCRβ constant region intracellular domain; wherein the intracellular signaling domain does not contain a functional CD3ζ signaling domain.

In some embodiments, the chimeric antigen receptor (CAR) does not contain a T cell receptor α (TCRα) variable region or a T cell receptor β (TCRβ) variable region. In some embodiments, the antigen binding domain, transmembrane domain, and intracellular signaling domain are operatively linked. In some embodiments, the CAR further comprises a TCRβ 1 constant domain or a TCRβ 2 constant domain. In some embodiments, the transmembrane domain comprises a TCRβ constant 1 domain or a TCRβ constant 2 domain. In some embodiments, the antigen binding domain is connected to the transmembrane domain by a linker. In some embodiments, the TCRβ constant intracellular domain comprises a TCRβ constant 1 intracellular domain or a TCRβ constant 2 intracellular domain. In some embodiments, the intracellular signaling domain further comprises a costimulatory signaling domain. In some embodiments, the antigen binding domain is a human or humanized single chain variable fragment (scFv) or single domain antibody (sdAb). In some embodiments, the antigen binding domain specifically binds to a tumor associated antigen. In some embodiments, the encoded chimeric antigen receptor (CAR) is expressed in frame and as a single polypeptide chain.

In one aspect, provided herein are recombinant nucleic acids encoding a chimeric antigen receptor (CAR), wherein the CAR comprises (a) an antigen binding domain, wherein the antigen binding domain is a single chain variable fragment (scFv) or a single domain antibody; (b) a transmembrane domain; and (c) an intracellular signaling domain comprising a TCRβ intracellular domain; wherein the intracellular signaling domain does not contain a functional CD3 signaling domain.

In some embodiments, the chimeric antigen receptor (CAR) does not contain a T cell receptor α (TCRα) variable region or a T cell receptor β (TCRβ) variable region. In some embodiments, the antigen binding domain, transmembrane domain, and intracellular signaling domain are operatively linked. In some embodiments, the CAR further comprises a TCRβ 1 constant domain or a TCRβ 2 constant domain. In some embodiments, the transmembrane domain comprises a TCRβ constant 1 domain or a TCRβ constant 2 domain. In some embodiments, the antigen binding domain is connected to the transmembrane domain by a linker. In some embodiments, the TCRβ constant intracellular domain comprises a TCRβ constant 1 intracellular domain or a TCRβ constant 2 intracellular domain. In some embodiments, the intracellular signaling domain further comprises a costimulatory signaling domain. In some embodiments, the antigen binding domain is a human or humanized single chain variable fragment (scFv) or single domain antibody (sdAb). In some embodiments, the antigen binding domain specifically binds to a tumor associated antigen. In some embodiments, the encoded chimeric antigen receptor (CAR) is expressed in frame and as a single polypeptide chain.

In one aspect, provided herein are polypeptides encoded by a recombinant nucleic acid described herein.

In one aspect, provided herein are vectors comprising a recombinant nucleic acid molecule described herein.

In one aspect, provided herein are methods of making a population of immune effector cells, comprising transducing a plurality of immune effector cells with a vector described herein.

In one aspect, provided herein are populations of immune effector cells, wherein the immune effector cells comprise a recombinant nucleic acid described herein.

In some embodiments, the population of immune effector cells are made by the method described herein.

In some embodiments, the population of immune effector cells upon binding of the antigen binding domain of the CAR to a cognate antigen expressed by a cell, the level of expression of at least one proinflammatory cytokine by the population immune effector cells is lower relative to the level of expression of the at least one proinflammatory cytokine by a comparable population of immune effector cells that comprise a nucleic acid encoding a CAR that comprises a CD3ζ intracellular signaling domain.

In some embodiments, the population of immune effector cells upon binding of the antigen binding domain of the CAR to a cognate antigen expressed by a cell, the level of expression of at least one proinflammatory cytokine by the population of immune effector cells is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression of the at least one proinflammatory cytokine by a comparable population of immune effector cells that comprise a nucleic acid encoding a CAR that comprises a CD3ζ intracellular signaling domain.

In some embodiments, the population of immune effector cells upon binding of the antigen binding domain of the CAR to a cognate antigen expressed by a cell in the presence of a population of antigen presenting cells, the level of expression of at least one proinflammatory cytokine by the population of antigen presenting cells is lower relative to the level of expression of the at least one proinflammatory cytokine by a comparable population of antigen presenting cells in the presence of a comparable population of immune effector cells that comprise a nucleic acid encoding a CAR that comprises a CD3ζ intracellular signaling domain.

In some embodiments, the population of immune effector cells upon binding of the antigen binding domain of the CAR to a cognate antigen expressed by a cell in the presence of a population of antigen presenting cells, the level of expression of at least one proinflammatory cytokine by the antigen presenting cell is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression of the at least one proinflammatory cytokine by a comparable population of antigen presenting cells in the presence of a population of comparable immune effector cells that comprise a nucleic acid encoding a CAR that comprises a CD3ζ intracellular signaling domain.

In some embodiments, the at least one proinflammatory cytokine is selected from the group consisting of IFNγ, TNFα, IL-6, IL-1β, IL-8, IL-10, IL-17, sIL2Rα, sgp130, sIL6R, MCP1, MIP1α, MIP1β, and GM-CSF.

In some embodiments, expression of the at least one proinflammatory cytokine is measured by determining the level of the cytokine secreted from the population of immune effector cells, as measured by an assay described herein.

In some embodiments, expression of the at least one proinflammatory cytokine is measured by determining the level of the cytokine secreted from the population of antigen presenting cells, as measured by an assay described herein.

In some embodiments, said population of antigen presenting cells comprises dendritic cells, macrophages, or monocytes.

In one aspect, provided herein are pharmaceutical compositions comprising at least a portion of the population of immune effector cells described herein.

In one aspect, provided herein are methods of treating a cancer in a subject, the method comprising: administering to the subject at least a portion of the population of immune effector cells described herein.

In one aspect, provided herein are methods of preventing or lessening the severity of cytokine release syndrome (CRS) in a human subject, the method comprising: administering to the subject at least a portion of the population of immune effector cells described herein.

In some embodiments, the subject has cancer.

In some embodiments, the subject does not exhibit at least one symptom of CRS (e.g., as described herein) within 1, 2, 3, 4, 5, 6, 7, 10, 14, 21, or 30 days of administration of the immune cell.

In some embodiments, the subject does not exhibit at least one symptom grade 4 or grade 5 CRS (e.g., as described herein).

In some embodiments, the subject does not exhibit any symptom grade 4 or grade 5 CRS (e.g., as described herein).

In some embodiments, the level of one or more protein selected from the group consisting of IL-6, IL-1β, IL-8, IL-10, IFNγ, TNFα, sIL2Rα, sgp130, sIL6R, MCP1, MIP1α, MIP1β, and GM-CSF, in the serum of the subject post administration (e.g., 1 hour, 6 hours, 12 hours, 1 day, 2 days, 3 days, 4 days, 5, days, 6 days, 7 days, 10 days, 14 days, 21 days, 30 days) of the cell (e.g., population of cells, e.g., population of immune effector cells) is within ±20%, ±15%, ±10%, ±9%, ±8%, ±7%, ±6%, ±5%, ±4%, ±3%, ±2% or ±1% of the level of the one or more protein in the serum of the subject prior to administration (e.g., 10 minutes, 30 minutes, 1 hour, 6 hours, 12 hours, 24 hours, 48 hours, 72 hours) of the immune cell.

In some embodiments, the method further comprises selecting the subject for administration of the immune cell of any one of claims 86-100 based on a determination of at least one of the following: the subject's risk of developing CRS, the subject's risk of developing CRS if administered a cell expressing a CAR comprising a CD3ζ signaling domain, the subject's diagnosis of CRS, or the subject's diagnosis of CRS associated with or induced by administration of a cell expressing a CAR comprising a CD3ζ signaling domain.

In some embodiments, the subject is selected for administration if the subject is at risk of developing CRS, if the subject is at risk of developing CRS if administered a CAR comprising a cell expressing a CAR CD3ζ signaling domain, if the subject has been diagnosed with CRS, or if the subject has been diagnosed with CRS associated with or induced by administration of a cell expressing a CAR comprising a CD3ζ signaling domain.

In some embodiments, the cell is autologous or allogenic to the subject administered said cell.

In some embodiments, the cancer is a solid cancer or hematological cancer.

In some embodiments, the cancer is a solid cancer.

In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer.

In some embodiments, the cancer is a hematologic cancer.

In some embodiments, the hematologic cancer is a leukemia, lymphoma, or myeloma.

In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

In one aspect, provided herein are recombinant nucleic acids encoding an exogenous T cell receptor (TCR), wherein the TCR comprises: a TCRα chain comprising i) an immunoglobulin variable heavy domain, ii) a TCRα transmembrane domain, and iii) an intracellular signaling domain comprising optionally a TCRα intracellular domain; a TCRβ chain comprising i) an immunoglobulin variable light domain, ii) a TCRβ transmembrane domain, and iii) an intracellular signaling domain comprising a TCRβ intracellular domain; wherein the immunoglobulin variable heavy domain and the immunoglobulin variable light domain form an antigen binding domain; wherein the TCR does not contain a functional CD3ζ intracellular signaling domain; and wherein the TCR does not comprise a T cell receptor α (TCRα) variable region or a T cell receptor β (TCRβ) variable region.

In some embodiments, the TCRα chain further comprises a TCRα constant domain.

In one aspect, provided herein are recombinant nucleic acids encoding an exogenous T cell receptor (TCR), wherein the TCR comprises: a TCRα chain comprising i) an immunoglobulin variable light domain, ii) a TCRα transmembrane domain, and iii) an intracellular signaling domain comprising optionally a TCRα intracellular domain; a TCRβ chain comprising i) an immunoglobulin variable heavy domain, ii) a TCRβ transmembrane domain, and iii) an intracellular signaling domain comprising a TCRβ intracellular domain; wherein the immunoglobulin variable heavy domain and the immunoglobulin variable light domain form an antigen binding domain; wherein the TCR does not contain a functional CD3ζ intracellular signaling domain; and wherein the TCR does not comprise a T cell receptor α (TCRα) variable region or a T cell receptor β (TCRβ) variable region.

In some embodiments, the TCRα chain further comprises a TCRα constant domain.

In one aspect, provided herein are recombinant nucleic acids encoding an exogenous T cell receptor (TCR), wherein the TCR comprises: a TCRα chain comprising i) an antigen binding domain (e.g., a scFv), ii) a TCRα variable domain, iii) a TCRα constant domain, iv) a TCRα transmembrane domain, and iii) an intracellular signaling domain comprising optionally a TCRα intracellular domain; a TCRβ chain comprising i) an TCRβ variable domain, ii) a TCRβ constant domain, iii) a TCRβ transmembrane domain, and iv) an intracellular signaling domain comprising a TCRβ intracellular domain; and wherein the TCR does not contain a functional CD3ζ intracellular signaling domain.

In one aspect, provided herein are recombinant nucleic acids encoding an exogenous T cell receptor (TCR), wherein the TCR comprises: a TCRα chain comprising i) a TCRα variable domain, ii) a TCRα constant domain, iii) a TCRα transmembrane domain, and iv) an intracellular signaling domain comprising optionally a TCRα intracellular domain; a TCRβ chain comprising i) an antigen binding domain (e.g., a scFv), ii) an TCRβ variable domain, iii) a TCRβ constant domain, iii) a TCRβ transmembrane domain, and iv) an intracellular signaling domain comprising a TCRβ intracellular domain; and wherein the TCR does not contain a functional CD3ζ intracellular signaling domain.

In one aspect, provided herein are polypeptides encoded by the recombinant nucleic acid described herein.

In one aspect, provided herein are vectors comprising the recombinant nucleic acid described herein.

In one aspect, provided herein are methods of making a population of immune effector cells, comprising transducing the population of immune effector cells with a vector described herein.

In one aspect, provided herein are populations of immune effector cells, wherein the immune effector cells comprise a recombinant nucleic acid described herein.

In some embodiments, the immune effector cells are made by the method described herein.

In some embodiments, the immune effector cells upon binding of the antigen binding domain of the TCR to a cognate antigen expressed by a cell, the level of expression of at least one proinflammatory cytokine by the population immune effector cells is lower relative to the level of expression of the at least one proinflammatory cytokine by a comparable population of immune effector cells that comprise a nucleic acid encoding a TCR that comprises a CD3ζ intracellular signaling domain.

In some embodiments, the immune effector cells upon binding of the antigen binding domain of the TCR to a cognate antigen expressed by a cell, the level of expression of at least one proinflammatory cytokine by the population of immune effector cells is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression of the at least one proinflammatory cytokine by a comparable population of immune effector cells that comprise a nucleic acid encoding a TCR that comprises a CD3 intracellular signaling domain.

In some embodiments, the immune effector cells upon binding of the antigen binding domain of the TCR to a cognate antigen expressed by a cell in the presence of a population of antigen presenting cells, the level of expression of at least one proinflammatory cytokine by the population of antigen presenting cells is lower relative to the level of expression of the at least one proinflammatory cytokine by a comparable population of antigen presenting cells in the presence of a comparable population of immune effector cells that comprise a nucleic acid encoding a TCR that comprises a CD3ζ intracellular signaling domain.

In some embodiments, the immune effector cells upon binding of the antigen binding domain of the TCR to a cognate antigen expressed by a cell in the presence of a population of antigen presenting cells, the level of expression of at least one proinflammatory cytokine by the antigen presenting cell is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression of the at least one proinflammatory cytokine by a comparable population of antigen presenting cells in the presence of a population of comparable immune effector cells that comprise a nucleic acid encoding a TCR that comprises a CD3ζ intracellular signaling domain.

In some embodiments, the at least one proinflammatory cytokine is selected from the group consisting of IFNγ, TNFα, IL-6, IL-1β, IL-8, IL-10, IL-17, sIL2Rα, sgp130, sIL6R, MCP1, MIP1α, MIP1β, and GM-CSF.

In some embodiments, expression of the at least one proinflammatory cytokine is measured by determining the level of the cytokine secreted from the population of immune effector cells, as measured by an assay described herein.

In some embodiments, expression of the at least one proinflammatory cytokine is measured by determining the level of the cytokine secreted from the population of antigen presenting cells, as measured by an assay described herein.

In some embodiments, said population of antigen presenting cells comprises dendritic cells, macrophages, or monocytes.

In one aspect, provided herein are pharmaceutical compositions comprising at least a portion of the population of immune effector cells described herein.

In one aspect, provided herein are methods of treating a cancer in a subject, the method comprising: administering to the subject at least a portion of the population of immune effector cells described herein.

In one aspect, provided herein are methods of preventing or lessening the severity of cytokine release syndrome (CRS) in a human subject, the method comprising: administering to the subject at least a portion of the population of immune effector cells described herein.

In some embodiments, the subject has cancer.

In some embodiments, the subject does not exhibit at least one symptom of CRS (e.g., as described herein) within 1, 2, 3, 4, 5, 6, 7, 10, 14, 21, or 30 days of administration of the immune cell.

In some embodiments, the subject does not exhibit at least one symptom grade 4 or grade 5 CRS (e.g., as described herein).

In some embodiments, the subject does not exhibit any symptom grade 4 or grade 5 CRS (e.g., as described herein).

In some embodiments, the level of one or more protein selected from the group consisting of IL-6, IL-10, IL-8, IL-10, IFNγ, TNFα, sIL2Rα, sgp130, sIL6R, MCP1, MIP1α, MIP1β, and GM-CSF, in the serum of the subject post administration (e.g., 1 hour, 6 hours, 12 hours, 1 day, 2 days, 3 days, 4 days, 5, days, 6 days, 7 days, 10 days, 14 days, 21 days, 30 days) of the cell (e.g., population of cells, e.g., population of immune effector cells) is within ±20%, ±15%, ±10%, ±9%, ±8%, ±7%, ±6%, ±5%, ±4%, ±3%, ±2% or ±1% of the level of the one or more protein in the serum of the subject prior to administration (e.g., 10 minutes, 30 minutes, 1 hour, 6 hours, 12 hours, 24 hours, 48 hours, 72 hours) of the immune cell.

In some embodiments, the method further comprises selecting the subject for administration of the immune cell described herein based on a determination of at least one of the following: the subject's risk of developing CRS, the subject's risk of developing CRS if administered a cell expressing a CAR comprising a CD3ζ signaling domain, the subject's diagnosis of CRS, or the subject's diagnosis of CRS associated with or induced by administration of a cell expressing a CAR comprising a CD3ζ signaling domain.

In some embodiments, the subject is selected for administration if the subject is at risk of developing CRS, if the subject is at risk of developing CRS if administered a CAR comprising a cell expressing a CAR CD3ζ signaling domain, if the subject has been diagnosed with CRS, or if the subject has been diagnosed with CRS associated with or induced by administration of a cell expressing a CAR comprising a CD3ζ signaling domain.

In some embodiments, the cell is autologous or allogenic to the subject administered said cell.

In some embodiments, the cancer is a solid cancer or hematological cancer.

In some embodiments, the cancer is a solid cancer.

In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer.

In some embodiments, the cancer is a hematologic cancer.

In some embodiments, the hematologic cancer is a leukemia, lymphoma, or myeloma.

In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

In one aspect, provided herein are methods of expanding a T cell population ex vivo comprising contacting the T cell population with one or more anti-TCRβV antibody, and methods of treating a disease or disorder, e.g., cancer, using the aforesaid expanded cell populations.

Methods described herein include, methods of activating or expanding (or both activating and expanding) T cells ex vivo comprising contacting a plurality of T cells to a first agent, wherein the first agent comprises a first domain that specifically binds to a T cell receptor beta variable chain (TCRβV) region, thereby generating a first population of T cells.

In some embodiments, the method further comprises contacting the plurality of T cells with a second agent, wherein the second agent comprises a domain that specifically binds to a T cell receptor beta variable chain (TCRβV) region, wherein the first and the second agents specifically bind to different TCRβV regions.

In some embodiments, the first agent comprises a domain that specifically binds to a TCRβV region of a first TCRβV, and the second agent comprises a domain that specifically binds to a TCRβV region of a second TCRβV, wherein the first and the second TCRβVs belong to different TCRβV subfamilies or are different members of the same TCRβV subfamily.

In some embodiments, the first agent comprises a domain that specifically binds to a TCRβV region of a first TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily; the second agent comprises a domain that specifically binds to a second TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and wherein the first and the second agent each specifically bind a TCRβV belonging to a different subfamily or different members of the same TCRβV subfamily.

In some embodiments, the first and the second agent each specifically bind a TCRβV belonging to a different subfamily. In some embodiments, the first and the second agent each specifically bind different members of the same TCRβV subfamily.

In some embodiments, the methods further comprise contacting the plurality of T cells with one more (e.g., 1, 2, 3, 4, 5, 6, 7, 8, 9, 10, or more) agents, wherein each of the one or more agents comprises a domain that specifically binds to a T cell receptor beta variable chain (TCRβV) region, wherein each of the one or more agents specifically binds to a different T cell receptor beta variable chain (TCRβV) region, and wherein each one of the TCRβV regions the one or more agents specifically bind is different from the TCRβV regions the first and the second agents specifically bind.

In some embodiments, each of the one or more (e.g., 1, 2, 3, 4, 5, 6, 7, 8, 9, 10, or more) agents specifically bind to a TCRβV belonging to different TCRβV subfamily or that are different members of the same TCRβV subfamily; and wherein each of the one or more agents specifically bind to a TCRβV belonging to different TCRβV subfamily than the TCRβVs bound by the first agent and the second agent or each of the one or more agents specifically bind different members of the same TCRβV subfamily as the TCRβVs bound by the first agent, the second agent, or both.

In some embodiments, the one or more (e.g., 1, 2, 3, 4, 5, 6, 7, 8, 9, 10, or more) agents each comprise a domain that specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily.

In some embodiments, each of the one or more (e.g., 1, 2, 3, 4, 5, 6, 7, 8, 9, 10, or more) agents specifically bind a TCRβV belonging to a different subfamily, and wherein each of the one or more agents specifically bind a TCRβV that belongs to a different subfamily than the TCRβVs bound by the first agent and the second agent.

In some embodiments, the first agent further comprises a second domain that binds to a protein expressed on the surface of one more T-cells in the plurality. In some embodiments, the first agent is a bispecific antibody molecule.

In some embodiments, the second domain specifically binds to a T cell receptor beta variable chain (TCRβV) region. In some embodiments, the second domain and the first domain specifically bind different T cell receptor beta variable chain (TCRβV) regions. In some embodiments, the second domain and the first domain specifically bind TCRβVs belonging to different subfamilies or different members of the same TCRβV subfamily. In some embodiments, the first domain specifically binds specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and the second domain specifically binds a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the second domain and the first domain specifically bind TCRβVs belonging to different subfamilies. In some embodiments, the second domain and the first domain specifically bind different members of the same TCRβV subfamily.

In some embodiments, the second domain specifically binds to CD19 or 4-1BB.

In some embodiments, the second domain specifically binds to an antibody molecule. In some embodiments, the antibody molecule is expressed by one or more of the T cells in the plurality. In some embodiments, the antibody molecule comprises a variable heavy chain and a variable light chain. In some embodiments, the antibody molecule is a scFv or a Fab. In some embodiments, the second domain specifically binds to a light chain of the antibody molecule. In some embodiments, the second domain specifically binds to a κ light chain region of an antibody molecule. In some embodiments, the second domain comprises a protein L.

In some embodiments, the first population of T cells exhibit one or more of: (i) reduced expression of IL-1β, (ii) reduced expression level of IL-6, (iii) reduced expression of TNFα, (iv) increased expression of IL-2, (v) increased expression of IFNγ, (vi) maintained expression of IFNγ, and (vii) increased expression of 4-1BB, relative to a plurality of T cells contacted with an agent comprising a domain that specifically binds CD3 (e.g., CDRε).

In some embodiments, the contacting comprises incubating the plurality of T cells with the first agent.

In some embodiments, contacting comprises incubating or culturing the plurality of T cells with the first agent for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days. In some embodiments, contacting comprises incubating or culturing the plurality of T cells with the first agent for at most about 10 minutes, 20 minutes, 30 minutes, 1 hour, 6 hours, 10 hours, 12 hours, 24 hours, 36 hours, 48 hours, 72 hours, 5 days, 7 days, 10 days, 12 days, 14 days, 15 days, 21 days, 30 days, 45 days, or 60 days. In some embodiments, contacting comprises incubating or culturing the plurality of T cells with the first agent for about from 10-90 minutes, 10-60 minutes, 10-30 minutes, 1-30 days, 1-21 days, 1-14 days, 1-7 days, 1-5 days, 1-3 days, 21-30 days, 14-30 days, 7-30 days, 5-30 days, or 3-30 days.

In some embodiments, the first agent is coupled to a solid surface (e.g., a bead). In some embodiments, the first agent comprises an antibody domain.

In some embodiments, the first agent comprises an anti-idiotypic antibody domain. In some embodiments, the first agent comprises a mouse antibody domain. In some embodiments, the first agent comprises a human antibody domain. In some embodiments, the first agent comprises a humanized antibody domain. In some embodiments, the first agent comprises an antigen binding domain comprising a single chain Fv (scFv) or a Fab. In some embodiments, the first agent comprises an antibody comprising two antibody heavy chains, each heavy chain comprising a variable region and a constant region; and two antibody light chains, each light chain comprising a variable region and a constant region.

In some embodiments, the plurality of T cells comprises one or more T cell comprising an exogenous nucleic acid.

In some embodiments, the plurality of T cells comprises one or more T cell comprising an exogenous nucleic acid encoding a chimeric polypeptide. In some embodiments, the method comprises introducing an exogenous nucleic acid into one or more T cells of the plurality prior to contacting the plurality of T cells with the first agent. In some embodiments, the method comprises introducing an exogenous nucleic acid into one or more T cells of the plurality after to contacting the plurality of T cells with the first agent. In some embodiments, the method further comprises introducing an exogenous nucleic acid encoding a chimeric polypeptide into one or more of the T cells of the plurality prior to contacting the plurality of T cells with the first agent. In some embodiments, the method further comprises introducing an exogenous nucleic acid encoding a chimeric polypeptide into one or more of the T cells of the plurality after to contacting the plurality of T cells with the first agent. In some embodiments, the exogenous nucleic acid is introduced by transduction or transfection.

In some embodiments, the chimeric polypeptide is a chimeric antigen receptor (CAR). In some embodiments, the chimeric antigen receptor (CAR) comprises and antigen binding region, a transmembrane region, and an intracellular signaling region. In some embodiments, the intracellular signaling region comprises one or more signaling domain. In some embodiments, the intracellular signaling domain comprises a signaling domain from CD27, CD28, 4-1BB, ICOS, OX40, DAP10, DAP12, CD134, CD3-zeta or fragment or combination thereof. In some embodiments, the transmembrane region comprises a transmembrane region from CD8, CD28, or CTLA4.

In some embodiments, the antigen binding region comprises an antibody domain. In some embodiments, the antibody domain comprises a scFv or a Fab. In some embodiments, the antigen binding region specifically binds a tumor associated antigen (e.g., as described herein).

In some embodiments, the chimeric polypeptide is a chimeric T cell receptor (TCR). In some embodiments, the chimeric TCR comprises an antigen binding region. In some embodiments, the chimeric TCR further comprises a transmembrane region. In some embodiments, the chimeric TCR further comprises an intracellular signaling region. In some embodiments, the chimeric TCR comprises a TCR α polypeptide and a TCR β polypeptide. In some embodiments, chimeric TCR comprises a TCR γ polypeptide and a TCR δ polypeptide. In some embodiments, the antigen binding region specifically binds a tumor associated antigen.

In some embodiments, the plurality of T cells comprises one or more T cells from a human subject.

In some embodiments, the one or more T cell are removed from the human subject via apheresis.

In some embodiments, the plurality of T cells comprises one or more T cell from a human subject that is healthy (e.g., a subject that does not have or has not been diagnosed with a specified disease or condition, e.g., a cancer). In some embodiments, the plurality of T cells comprises one or more T cells from a mammalian (e.g., human) subject having or diagnosed with a disease or condition (e.g., diagnosed with a specified disease or condition, e.g., cancer). In some embodiments, the disease is a cancer. In some embodiments, the cancer is a solid tumor or hematological cancer. In some embodiments, the cancer is selected from the group consisting of leukemia, lymphoma, myeloma, prostate, lung, renal, stomach, colon, ovarian, bladder, breast, cervical, esophageal, testicular, liver, pancreatic, rectal, thyroid, uterine, skin, muscle, cartilage, bone, endothelial, epithelial, dermal, basal, retinal, skin, or brain.

In some embodiments, the plurality of T cells comprises one or more autologous T cell. In some embodiments, the plurality of T cells comprises one or more allogeneic T cell.

In some embodiments, the number of cells in the first T cell population is at least 1, 2, 3, 4, 5, 6, 7, 8, 9, 10, 50, 100, or 1000 fold greater than the number of cells in the plurality of T cells prior to be contacted with the first agent.

In some embodiments, the agent that specifically binds CD3 (e.g., CD3ε) comprises an antibody domain (e.g., an anti-CD3 antibody (e.g., an anti-CD3ε antibody)).

In some embodiments, the agent that specifically binds CD3 specifically binds CD3ε.

In some embodiments, the first agent, upon binding to the TCRβV region, results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, the first agent, upon binding to the TCRβV region, results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., T effector memory (TEM) cells, e.g., TEM cells expressing CD45RA (TEMRA) cells.

In some embodiments, expansion of a population of memory effector T cells, e.g., TEM cells, e.g., TEMRA cells, in the first population of T cells a is increased compared to expansion of a similar population of cells with an antibody that binds to a CD3 molecule.

In some embodiments, the population of expanded T effector memory cells comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7−, CD95+ T cells, optionally wherein the T cells comprise CD3+, CD4+ or CD8+ T cells.

In some embodiments, binding of the first agent to the TCRβV region results in one, two, three, four, five, six, seven, eight, nine, ten or more (e.g., all) of the following: (i) reduced level, e.g., expression level, and/or activity of IL-10; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, binding of the first agent to the TCRβV region results in a reduction of at least 2, 5, 10, 20, 50, 100, or 200 fold, or at least 2-200 fold (e.g., 5-150, 10-100, 20-50 fold) in the expression level and or activity of IL-1β as measured by an assay described herein.

In some embodiments, binding of the first agent to the TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay described herein.

In some embodiments, binding of the first agent to the TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay described herein.

In some embodiments, binding of the first agent to the TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay described herein.

Methods described herein, include, methods of expanding T cells ex vivo comprising contacting a plurality of T cells to a plurality of agents, wherein the plurality of agents comprises two, three, four, five, or more agents, wherein each agent of the plurality comprises a domain that specifically binds to a different T cell receptor beta variable chain (TCRβV) region, thereby generating a first population of T cells.

In some embodiments, each agent of the plurality specifically binds to a different TCRβV, wherein each TCRβV belongs to a different TCRβV subfamily or are different members of the same TCRβV subfamily.

In some embodiments, each agent of the plurality comprises a domain that specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily.

In some embodiments, each agent of the plurality specifically binds to a different TCRβV, wherein each TCRβV belongs to a different TCRβV subfamily.

Methods described herein, include, methods of expanding T cells ex vivo comprising contacting a plurality of T cells to a plurality of agents, wherein the plurality of agents comprises at least a first and a second agent, wherein each agent of the plurality comprises a domain that specifically binds to a different T cell receptor beta variable chain (TCRβV) region, thereby generating a first population of T cells.

In some embodiments, the plurality comprises at least 3, 4, 5, 6, 7, 8, 9, 10, or more agents.

In some embodiments, the method further comprises contacting the plurality of T cells with a second agent, wherein the second agent comprises a domain that specifically binds to a T cell receptor beta variable chain (TCRβV) region, wherein the first and the second agents specifically bind to different TCRβV regions.

In some embodiments, the first agent comprises a domain that specifically binds to a TCRβV region of a first TCRβV, and the second agent comprises a domain that specifically binds to a TCRβV region of a second TCRβV, wherein the first and the second TCRβVs belong to different TCRβV subfamilies or are different members of the same TCRβV subfamily.

In some embodiments, the first agent comprises a domain that specifically binds to a TCRβV region of a first TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily; the second agent comprises a domain that specifically binds to a second TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and wherein the first and the second agent each specifically bind a TCRβV belonging to a different subfamily or different members of the same TCRβV subfamily.

In some embodiments, the first and the second agent each specifically bind a TCRβV belonging to a different subfamily. In some embodiments, the first and the second agent each specifically bind different members of the same TCRβV subfamily.

In some embodiments, the methods further comprise contacting the plurality of T cells with one more (e.g., 1, 2, 3, 4, 5, 6, 7, 8, 9, 10, or more) agents, wherein each of the one or more agents comprises a domain that specifically binds to a T cell receptor beta variable chain (TCRβV) region, wherein each of the one or more agents specifically binds to a different T cell receptor beta variable chain (TCRβV) region, and wherein each one of the TCRβV regions the one or more agents specifically bind is different from the TCRβV regions the first and the second agents specifically bind.

In some embodiments, each of the one or more (e.g., 1, 2, 3, 4, 5, 6, 7, 8, 9, 10, or more) agents specifically bind to a TCRβV belonging to different TCRβV subfamily or that are different members of the same TCRβV subfamily; and wherein each of the one or more agents specifically bind to a TCRβV belonging to different TCRβV subfamily than the TCRβVs bound by the first agent and the second agent or each of the one or more agents specifically bind different members of the same TCRβV subfamily as the TCRβVs bound by the first agent, the second agent, or both.

In some embodiments, the one or more (e.g., 1, 2, 3, 4, 5, 6, 7, 8, 9, 10, or more) agents each comprise a domain that specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily.

In some embodiments, each of the one or more (e.g., 1, 2, 3, 4, 5, 6, 7, 8, 9, 10, or more) agents specifically bind a TCRβV belonging to a different subfamily, and wherein each of the one or more agents specifically bind a TCRβV that belongs to a different subfamily than the TCRβVs bound by the first agent and the second agent.

In some embodiments, the first agent and/or the second agent further comprises a second domain that binds to a protein expressed on the surface of one more T-cells in the plurality. In some embodiments, the first agent is a bispecific antibody molecule.

In some embodiments, the second domain specifically binds to a T cell receptor beta variable chain (TCRβV) region. In some embodiments, the second domain and the first domain specifically bind different T cell receptor beta variable chain (TCRβV) regions. In some embodiments, the second domain and the first domain specifically bind TCRβVs belonging to different subfamilies or different members of the same TCRβV subfamily. In some embodiments, the first domain specifically binds specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and the second domain specifically binds a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the second domain and the first domain specifically bind TCRβVs belonging to different subfamilies. In some embodiments, the second domain and the first domain specifically bind different members of the same TCRβV subfamily.

In some embodiments, the second domain specifically binds to CD19 or 4-1BB.

In some embodiments, the second domain specifically binds to an antibody molecule. In some embodiments, the antibody molecule is expressed by one or more of the T cells in the plurality. In some embodiments, the antibody molecule comprises a variable heavy chain and a variable light chain. In some embodiments, the antibody molecule is a scFv or a Fab. In some embodiments, the second domain specifically binds to a light chain of the antibody molecule. In some embodiments, the second domain specifically binds to a κ light chain region of an antibody molecule. In some embodiments, the second domain comprises a protein L.

In some embodiments, the first population of T cells exhibit one or more of: (i) reduced expression of IL-1β, (ii) reduced expression level of IL-6, (iii) reduced expression of TNFα, (iv) increased expression of IL-2, (v) increased expression of IFNγ, (vi) maintained expression of IFNγ, and (vii) increased expression of 4-1BB, relative to a plurality of T cells contacted with an agent comprising a domain that specifically binds CD3 (e.g., CDR).

In some embodiments, the contacting comprises incubating the plurality of T cells with the first agent.

In some embodiments, contacting comprises incubating or culturing the plurality of T cells with the first agent for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days. In some embodiments, contacting comprises incubating or culturing the plurality of T cells with the first agent for at most about 10 minutes, 20 minutes, 30 minutes, 1 hour, 6 hours, 10 hours, 12 hours, 24 hours, 36 hours, 48 hours, 72 hours, 5 days, 7 days, 10 days, 12 days, 14 days, 15 days, 21 days, 30 days, 45 days, or 60 days. In some embodiments, contacting comprises incubating or culturing the plurality of T cells with the first agent for about from 10-90 minutes, 10-60 minutes, 10-30 minutes, 1-30 days, 1-21 days, 1-14 days, 1-7 days, 1-5 days, 1-3 days, 21-30 days, 14-30 days, 7-30 days, 5-30 days, or 3-30 days.

In some embodiments, the first agent is coupled to a solid surface (e.g., a bead). In some embodiments, the first agent comprises an antibody domain. In some embodiments, each agent of the plurality is coupled to one or more solid surface (e.g., one or more beads). In some embodiments, each agent of the plurality comprises an antibody domain.

In some embodiments, the first agent comprises an anti-idiotypic antibody domain. In some embodiments, the first agent comprises a mouse antibody domain. In some embodiments, the first agent comprises a human antibody domain. In some embodiments, the first agent comprises a humanized antibody domain. In some embodiments, the first agent comprises an antigen binding domain comprising a single chain Fv (scFv) or a Fab. In some embodiments, the first agent comprises an antibody comprising two antibody heavy chains, each heavy chain comprising a variable region and a constant region; and two antibody light chains, each light chain comprising a variable region and a constant region.

In some embodiments, each agent of the plurality comprises an anti-idiotypic antibody domain. In some embodiments, each agent of the plurality comprises a mouse antibody domain. In some embodiments, each agent of the plurality comprises a human antibody domain. In some embodiments, each agent of the plurality comprises a humanized antibody domain. In some embodiments, each agent of the plurality comprises an antigen binding domain comprising a single chain Fv (scFv) or a Fab. In some embodiments, each agent of the plurality comprises an antibody comprising two antibody heavy chains, each heavy chain comprising a variable region and a constant region; and two antibody light chains, each light chain comprising a variable region and a constant region.

In some embodiments, the plurality of T cells comprises one or more T cell comprising an exogenous nucleic acid.

In some embodiments, the plurality of T cells comprises one or more T cell comprising an exogenous nucleic acid encoding a chimeric polypeptide. In some embodiments, the method comprises introducing an exogenous nucleic acid into one or more T cells of the plurality prior to contacting the plurality of T cells with the first agent. In some embodiments, the method comprises introducing an exogenous nucleic acid into one or more T cells of the plurality after contacting the plurality of T cells with the first agent. In some embodiments, the method further comprises introducing an exogenous nucleic acid encoding a chimeric polypeptide into one or more of the T cells of the plurality prior to contacting the plurality of T cells with the first agent. In some embodiments, the method further comprises introducing an exogenous nucleic acid encoding a chimeric polypeptide into one or more of the T cells of the plurality after to contacting the plurality of T cells with the first agent. In some embodiments, the exogenous nucleic acid is introduced by transduction or transfection.

In some embodiments, the chimeric polypeptide is a chimeric antigen receptor (CAR). In some embodiments, the chimeric antigen receptor (CAR) comprises and antigen binding region, a transmembrane region, and an intracellular signaling region. In some embodiments, the intracellular signaling region comprises one or more signaling domain. In some embodiments, the intracellular signaling domain comprises a signaling domain from CD27, CD28, 4-1BB, ICOS, OX40, DAP10, DAP12, CD134, CD3-zeta or fragment or combination thereof. In some embodiments, the transmembrane region comprises a transmembrane region from CD8, CD28, or CTLA4.

In some embodiments, the antigen binding region comprises an antibody domain. In some embodiments, the antibody domain comprises a scFv or a Fab. In some embodiments, the antigen binding region specifically binds a tumor associated antigen.

In some embodiments, the chimeric polypeptide is a chimeric T cell receptor (TCR). In some embodiments, the chimeric TCR comprises an antigen binding region. In some embodiments, the chimeric TCR further comprises a transmembrane region. In some embodiments, the chimeric TCR further comprises an intracellular signaling region. In some embodiments, the chimeric TCR comprises a TCR α polypeptide and a TCR β polypeptide. In some embodiments, chimeric TCR comprises a TCR γ polypeptide and a TCR δ polypeptide. In some embodiments, the antigen binding region specifically binds a tumor associated antigen.

In some embodiments, the plurality of T cells comprises one or more T cells from a human subject. In some embodiments, the one or more T cell are removed from the human subject via apheresis. In some embodiments, the plurality of T cells comprises one or more T cell from a human subject that is healthy (e.g., a subject that does not have or has not been diagnosed with a specified disease or condition, e.g., a cancer). In some embodiments, the plurality of T cells comprises one or more T cells from a mammalian (e.g., human) subject having or diagnosed with a disease or condition (e.g., diagnosed with a specified disease or condition, e.g., cancer). In some embodiments, the disease is a cancer. In some embodiments, the cancer is a solid tumor or hematological cancer. In some embodiments, the cancer is selected from the group consisting of leukemia, lymphoma, myeloma, prostate, lung, renal, stomach, colon, ovarian, bladder, breast, cervical, esophageal, testicular, liver, pancreatic, rectal, thyroid, uterine, skin, muscle, cartilage, bone, endothelial, epithelial, dermal, basal, retinal, skin, or brain.

In some embodiments, the plurality of T cells comprises one or more autologous T cell. In some embodiments, the plurality of T cells comprises one or more allogeneic T cell.

In some embodiments, the number of cells in the first T cell population is at least 1, 2, 3, 4, 5, 6, 7, 8, 9, 10, 50, 100, or 1000 fold greater than the number of cells in the plurality of T cells prior to be contacted with the first agent.

In some embodiments, the agent that specifically binds CD3 (e.g., CD3ε) comprises an antibody domain (e.g., an anti-CD3 antibody (e.g., an anti-CD3ε antibody)).

In some embodiments, the agent that specifically binds CD3 specifically binds CD3ε.

In some embodiments, the first agent, upon binding to the TCRβV region, results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, the first agent, upon binding to the TCRβV region, results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., T effector memory (TEM) cells, e.g., TEM cells expressing CD45RA (TEMRA) cells.

In some embodiments, expansion of a population of memory effector T cells, e.g., TEM cells, e.g., TEMRA cells, in the first population of T cells a is increased compared to expansion of a similar population of cells with an antibody that binds to a CD3 molecule.

In some embodiments, the population of expanded T effector memory cells comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7−, CD95+ T cells, optionally wherein the T cells comprise CD3+, CD4+ or CD8+ T cells.

In some embodiments, binding of the first agent to the TCRβV region results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, binding of the first agent to the TCRβV region results in a reduction of at least 2, 5, 10, 20, 50, 100, or 200 fold, or at least 2-200 fold (e.g., 5-150, 10-100, 20-50 fold) in the expression level and or activity of IL-1β as measured by an assay described herein.

In some embodiments, binding of the first agent to the TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay described herein.

In some embodiments, binding of the first agent to the TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay described herein.

In some embodiments, binding of the first agent to the TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay described herein.

Methods disclosed herein, include, methods of treating cancer in a subject, the method comprising: removing a plurality of T cells from a human subject, expanding the plurality of T cells from the human subject comprising contacting the plurality of T cells to a first agent, wherein the first agent comprises a domain that specifically binds to a T cell receptor beta variable chain (TCRβV) region, thereby generating a first population of T cells, infusing at least a portion of the first population of T cells into the human subject, to thereby treat the cancer in the subject.

In some embodiments, the method further comprises contacting the plurality of T cells with a second agent, wherein the second agent comprises a domain that specifically binds to a T cell receptor beta variable chain (TCRβV) region, wherein the first and the second agents specifically bind to different TCRβV regions.

In some embodiments, the first agent comprises a domain that specifically binds to a TCRβV region of a first TCRβV, and the second agent comprises a domain that specifically binds to a TCRβV region of a second TCRβV, wherein the first and the second TCRβVs belong to different TCRβV subfamilies or are different members of the same TCRβV subfamily.

In some embodiments, the first agent comprises a domain that specifically binds to a TCRβV region of a first TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily; the second agent comprises a domain that specifically binds to a second TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and wherein the first and the second agent each specifically bind a TCRβV belonging to a different subfamily or different members of the same TCRβV subfamily.

In some embodiments, the first and the second agent each specifically bind a TCRβV belonging to a different subfamily. In some embodiments, the first and the second agent each specifically bind different members of the same TCRβV subfamily.

In some embodiments, the methods further comprise contacting the plurality of T cells with one more (e.g., 1, 2, 3, 4, 5, 6, 7, 8, 9, 10, or more) agents, wherein each of the one or more agents comprises a domain that specifically binds to a T cell receptor beta variable chain (TCRβV) region, wherein each of the one or more agents specifically binds to a different T cell receptor beta variable chain (TCRβV) region, and wherein each one of the TCRβV regions the one or more agents specifically bind is different from the TCRβV regions the first and the second agents specifically bind.

In some embodiments, each of the one or more (e.g., 1, 2, 3, 4, 5, 6, 7, 8, 9, 10, or more) agents specifically bind to a TCRβV belonging to different TCRβV subfamily or that are different members of the same TCRβV subfamily; and wherein each of the one or more agents specifically bind to a TCRβV belonging to different TCRβV subfamily than the TCRβVs bound by the first agent and the second agent or each of the one or more agents specifically bind different members of the same TCRβV subfamily as the TCRβVs bound by the first agent, the second agent, or both.

In some embodiments, the one or more (e.g., 1, 2, 3, 4, 5, 6, 7, 8, 9, 10, or more) agents each comprise a domain that specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily.

In some embodiments, each of the one or more (e.g., 1, 2, 3, 4, 5, 6, 7, 8, 9, 10, or more) agents specifically bind a TCRβV belonging to a different subfamily, and wherein each of the one or more agents specifically bind a TCRβV that belongs to a different subfamily than the TCRβVs bound by the first agent and the second agent.

In some embodiments, the first agent further comprises a second domain that binds to a protein expressed on the surface of one more T-cells in the plurality. In some embodiments, the first agent is a bispecific antibody molecule.

In some embodiments, the second domain specifically binds to a T cell receptor beta variable chain (TCRβV) region. In some embodiments, the second domain and the first domain specifically bind different T cell receptor beta variable chain (TCRβV) regions. In some embodiments, the second domain and the first domain specifically bind TCRβVs belonging to different subfamilies or different members of the same TCRβV subfamily. In some embodiments, the first domain specifically binds specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and the second domain specifically binds a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the second domain and the first domain specifically bind TCRβVs belonging to different subfamilies. In some embodiments, the second domain and the first domain specifically bind different members of the same TCRβV subfamily.

In some embodiments, the second domain specifically binds to CD19 or 4-1BB.

In some embodiments, the second domain specifically binds to an antibody molecule. In some embodiments, the antibody molecule is expressed by one or more of the T cells in the plurality. In some embodiments, the antibody molecule comprises a variable heavy chain and a variable light chain. In some embodiments, the antibody molecule is a scFv or a Fab. In some embodiments, the second domain specifically binds to a light chain of the antibody molecule. In some embodiments, the second domain specifically binds to a κ light chain region of an antibody molecule. In some embodiments, the second domain comprises a protein L.

In some embodiments, the first population of T cells exhibit one or more of: (i) reduced expression of IL-1β, (ii) reduced expression level of IL-6, (iii) reduced expression of TNFα, (iv) increased expression of IL-2, (v) increased expression of IFNγ, (vi) maintained expression of IFNγ, and (vii) increased expression of 4-1BB, relative to a plurality of T cells contacted with an agent comprising a domain that specifically binds CD3 (e.g., CDR).

In some embodiments, the contacting comprises incubating the plurality of T cells with the first agent.

In some embodiments, contacting comprises incubating or culturing the plurality of T cells with the first agent for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days. In some embodiments, contacting comprises incubating or culturing the plurality of T cells with the first agent for at most about 10 minutes, 20 minutes, 30 minutes, 1 hour, 6 hours, 10 hours, 12 hours, 24 hours, 36 hours, 48 hours, 72 hours, 5 days, 7 days, 10 days, 12 days, 14 days, 15 days, 21 days, 30 days, 45 days, or 60 days. In some embodiments, contacting comprises incubating or culturing the plurality of T cells with the first agent for about from 10-90 minutes, 10-60 minutes, 10-30 minutes, 1-30 days, 1-21 days, 1-14 days, 1-7 days, 1-5 days, 1-3 days, 21-30 days, 14-30 days, 7-30 days, 5-30 days, or 3-30 days.

In some embodiments, the first agent is coupled to a solid surface (e.g., a bead). In some embodiments, the first agent comprises an antibody domain.

In some embodiments, the first agent comprises an anti-idiotypic antibody domain. In some embodiments, the first agent comprises a mouse antibody domain. In some embodiments, the first agent comprises a human antibody domain. In some embodiments, the first agent comprises a humanized antibody domain. In some embodiments, the first agent comprises an antigen binding domain comprising a single chain Fv (scFv) or a Fab. In some embodiments, the first agent comprises an antibody comprising two antibody heavy chains, each heavy chain comprising a variable region and a constant region; and two antibody light chains, each light chain comprising a variable region and a constant region.

In some embodiments, the plurality of T cells comprises one or more T cell comprising an exogenous nucleic acid.

In some embodiments, the plurality of T cells comprises one or more T cell comprising an exogenous nucleic acid encoding a chimeric polypeptide. In some embodiments, the method comprises introducing an exogenous nucleic acid into one or more T cells of the plurality prior to contacting the plurality of T cells with the first agent. In some embodiments, the method comprises introducing an exogenous nucleic acid into one or more T cells of the plurality after to contacting the plurality of T cells with the first agent. In some embodiments, the method further comprises introducing an exogenous nucleic acid encoding a chimeric polypeptide into one or more of the T cells of the plurality prior to contacting the plurality of T cells with the first agent. In some embodiments, the method further comprises introducing an exogenous nucleic acid encoding a chimeric polypeptide into one or more of the T cells of the plurality after to contacting the plurality of T cells with the first agent. In some embodiments, the exogenous nucleic acid is introduced by transduction or transfection.

In some embodiments, the chimeric polypeptide is a chimeric antigen receptor (CAR). In some embodiments, the chimeric antigen receptor (CAR) comprises and antigen binding region, a transmembrane region, and an intracellular signaling region. In some embodiments, the intracellular signaling region comprises one or more signaling domain. In some embodiments, the intracellular signaling domain comprises a signaling domain from CD27, CD28, 4-1BB, ICOS, OX40, DAP10, DAP12, CD134, CD3-zeta or fragment or combination thereof. In some embodiments, the transmembrane region comprises a transmembrane region from CD8, CD28, or CTLA4.

In some embodiments, the antigen binding region comprises an antibody domain. In some embodiments, the antibody domain comprises a scFv or a Fab. In some embodiments, the antigen binding region specifically binds a tumor associated antigen.

In some embodiments, the chimeric polypeptide is a chimeric T cell receptor (TCR). In some embodiments, the chimeric TCR comprises an antigen binding region. In some embodiments, the chimeric TCR further comprises a transmembrane region. In some embodiments, the chimeric TCR further comprises an intracellular signaling region. In some embodiments, the chimeric TCR comprises a TCR α polypeptide and a TCR β polypeptide. In some embodiments, chimeric TCR comprises a TCR γ polypeptide and a TCR δ polypeptide. In some embodiments, the antigen binding region specifically binds a tumor associated antigen.

In some embodiments, the plurality of T cells comprises one or more T cells from a human subject. In some embodiments, the one or more T cell are removed from the human subject via apheresis. In some embodiments, the plurality of T cells comprises one or more T cell from a human subject that is healthy (e.g., a subject that does not have or has not been diagnosed with a specified disease or condition, e.g., a cancer). In some embodiments, the plurality of T cells comprises one or more T cells from a mammalian (e.g., human) subject having or diagnosed with a disease or condition (e.g., diagnosed with a specified disease or condition, e.g., cancer). In some embodiments, the disease is a cancer. In some embodiments, the cancer is a solid tumor or hematological cancer. In some embodiments, the cancer is selected from the group consisting of leukemia, lymphoma, myeloma, prostate, lung, renal, stomach, colon, ovarian, bladder, breast, cervical, esophageal, testicular, liver, pancreatic, rectal, thyroid, uterine, skin, muscle, cartilage, bone, endothelial, epithelial, dermal, basal, retinal, skin, or brain.

In some embodiments, the plurality of T cells comprises one or more autologous T cell. In some embodiments, the plurality of T cells comprises one or more allogeneic T cell.

In some embodiments, the number of cells in the first T cell population is at least 1, 2, 3, 4, 5, 6, 7, 8, 9, 10, 50, 100, or 1000 fold greater than the number of cells in the plurality of T cells prior to be contacted with the first agent.

In some embodiments, the agent that specifically binds CD3 (e.g., CD3ε) comprises an antibody domain (e.g., an anti-CD3 antibody (e.g., an anti-CD3ε antibody)).

In some embodiments, the agent that specifically binds CD3 specifically binds CD3ε.

In some embodiments, the first agent, upon binding to the TCRβV region, results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, the first agent, upon binding to the TCRβV region, results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., T effector memory (TEM) cells, e.g., TEM cells expressing CD45RA (TEMRA) cells.

In some embodiments, expansion of a population of memory effector T cells, e.g., TEM cells, e.g., TEMRA cells, in the first population of T cells a is increased compared to expansion of a similar population of cells with an antibody that binds to a CD3 molecule.

In some embodiments, the population of expanded T effector memory cells comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7−, CD95+ T cells, optionally wherein the T cells comprise CD3+, CD4+ or CD8+ T cells.

In some embodiments, binding of the first agent to the TCRβV region results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, binding of the first agent to the TCRβV region results in a reduction of at least 2, 5, 10, 20, 50, 100, or 200 fold, or at least 2-200 fold (e.g., 5-150, 10-100, 20-50 fold) in the expression level and or activity of IL-1β as measured by an assay described herein.

In some embodiments, binding of the first agent to the TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay described herein.

In some embodiments, binding of the first agent to the TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay described herein.

In some embodiments, binding of the first agent to the TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay described herein.

Methods described herein include, methods of preventing or lessening cytokine release syndrome (CRS) in a human subject, the method comprising: removing a plurality of T cells from a human subject, expanding the plurality of T cells from the human subject comprising contacting the plurality of T cells to a first agent, wherein the first agent comprises a domain that specifically binds to a T cell receptor beta variable chain (TCRβV) region, thereby generating a first population of T cells, infusing at least a portion of the first population of T cells into the human subject, wherein the subject shows no symptoms of CRS or less severe symptoms (e.g., one or more symptom described herein) of CRS relative to a human subject infused with at least a first population of T cells generated by removing a plurality of T cells the subject and expanding the plurality of T cells by contacting the plurality of T cells with an agent that binds CD3 (e.g., CD3e).

In some embodiments, the human subject has cancer.

In some embodiments, the first agent comprises a domain that specifically binds to a TCRβV region of a first TCRβV, and the second agent comprises a domain that specifically binds to a TCRβV region of a second TCRβV, wherein the first and the second TCRβVs belong to different TCRβV subfamilies or are different members of the same TCRβV subfamily.

In some embodiments, the first agent comprises a domain that specifically binds to a TCRβV region of a first TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily; the second agent comprises a domain that specifically binds to a second TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and wherein the first and the second agent each specifically bind a TCRβV belonging to a different subfamily or different members of the same TCRβV subfamily.

In some embodiments, the first and the second agent each specifically bind a TCRβV belonging to a different subfamily. In some embodiments, the first and the second agent each specifically bind different members of the same TCRβV subfamily.

In some embodiments, the methods further comprise contacting the plurality of T cells with one more (e.g., 1, 2, 3, 4, 5, 6, 7, 8, 9, 10, or more) agents, wherein each of the one or more agents comprises a domain that specifically binds to a T cell receptor beta variable chain (TCRβV) region, wherein each of the one or more agents specifically binds to a different T cell receptor beta variable chain (TCRβV) region, and wherein each one of the TCRβV regions the one or more agents specifically bind is different from the TCRβV regions the first and the second agents specifically bind.

In some embodiments, each of the one or more (e.g., 1, 2, 3, 4, 5, 6, 7, 8, 9, 10, or more) agents specifically bind to a TCRβV belonging to different TCRβV subfamily or that are different members of the same TCRβV subfamily; and wherein each of the one or more agents specifically bind to a TCRβV belonging to different TCRβV subfamily than the TCRβVs bound by the first agent and the second agent or each of the one or more agents specifically bind different members of the same TCRβV subfamily as the TCRβVs bound by the first agent, the second agent, or both.

In some embodiments, the one or more (e.g., 1, 2, 3, 4, 5, 6, 7, 8, 9, 10, or more) agents each comprise a domain that specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily.

In some embodiments, each of the one or more (e.g., 1, 2, 3, 4, 5, 6, 7, 8, 9, 10, or more) agents specifically bind a TCRβV belonging to a different subfamily, and wherein each of the one or more agents specifically bind a TCRβV that belongs to a different subfamily than the TCRβVs bound by the first agent and the second agent.

In some embodiments, the first agent further comprises a second domain that binds to a protein expressed on the surface of one more T-cells in the plurality. In some embodiments, the first agent is a bispecific antibody molecule.

In some embodiments, the second domain specifically binds to a T cell receptor beta variable chain (TCRβV) region. In some embodiments, the second domain and the first domain specifically bind different T cell receptor beta variable chain (TCRβV) regions. In some embodiments, the second domain and the first domain specifically bind TCRβVs belonging to different subfamilies or different members of the same TCRβV subfamily. In some embodiments, the first domain specifically binds specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and the second domain specifically binds a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the second domain and the first domain specifically bind TCRβVs belonging to different subfamilies. In some embodiments, the second domain and the first domain specifically bind different members of the same TCRβV subfamily.

In some embodiments, the second domain specifically binds to CD19 or 4-1BB.

In some embodiments, the second domain specifically binds to an antibody molecule. In some embodiments, the antibody molecule is expressed by one or more of the T cells in the plurality. In some embodiments, the antibody molecule comprises a variable heavy chain and a variable light chain. In some embodiments, the antibody molecule is a scFv or a Fab. In some embodiments, the second domain specifically binds to a light chain of the antibody molecule. In some embodiments, the second domain specifically binds to a κ light chain region of an antibody molecule. In some embodiments, the second domain comprises a protein L.

In some embodiments, the first population of T cells exhibit one or more of: (i) reduced expression of IL-1β, (ii) reduced expression level of IL-6, (iii) reduced expression of TNFα, (iv) increased expression of IL-2, (v) increased expression of IFNγ, (vi) maintained expression of IFNγ, and (vii) increased expression of 4-1BB, relative to a plurality of T cells contacted with an agent comprising a domain that specifically binds CD3 (e.g., CDR).

In some embodiments, the contacting comprises incubating the plurality of T cells with the first agent.

In some embodiments, contacting comprises incubating or culturing the plurality of T cells with the first agent for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days. In some embodiments, contacting comprises incubating or culturing the plurality of T cells with the first agent for at most about 10 minutes, 20 minutes, 30 minutes, 1 hour, 6 hours, 10 hours, 12 hours, 24 hours, 36 hours, 48 hours, 72 hours, 5 days, 7 days, 10 days, 12 days, 14 days, 15 days, 21 days, 30 days, 45 days, or 60 days. In some embodiments, contacting comprises incubating or culturing the plurality of T cells with the first agent for about from 10-90 minutes, 10-60 minutes, 10-30 minutes, 1-30 days, 1-21 days, 1-14 days, 1-7 days, 1-5 days, 1-3 days, 21-30 days, 14-30 days, 7-30 days, 5-30 days, or 3-30 days.

In some embodiments, the first agent is coupled to a solid surface (e.g., a bead). In some embodiments, the first agent comprises an antibody domain.

In some embodiments, the first agent comprises an anti-idiotypic antibody domain. In some embodiments, the first agent comprises a mouse antibody domain. In some embodiments, the first agent comprises a human antibody domain. In some embodiments, the first agent comprises a humanized antibody domain. In some embodiments, the first agent comprises an antigen binding domain comprising a single chain Fv (scFv) or a Fab. In some embodiments, the first agent comprises an antibody comprising two antibody heavy chains, each heavy chain comprising a variable region and a constant region; and two antibody light chains, each light chain comprising a variable region and a constant region.

In some embodiments, the plurality of T cells comprises one or more T cell comprising an exogenous nucleic acid.

In some embodiments, the plurality of T cells comprises one or more T cell comprising an exogenous nucleic acid encoding a chimeric polypeptide. In some embodiments, the method comprises introducing an exogenous nucleic acid into one or more T cells of the plurality prior to contacting the plurality of T cells with the first agent. In some embodiments, the method comprises introducing an exogenous nucleic acid into one or more T cells of the plurality after to contacting the plurality of T cells with the first agent. In some embodiments, the method further comprises introducing an exogenous nucleic acid encoding a chimeric polypeptide into one or more of the T cells of the plurality prior to contacting the plurality of T cells with the first agent. In some embodiments, the method further comprises introducing an exogenous nucleic acid encoding a chimeric polypeptide into one or more of the T cells of the plurality after to contacting the plurality of T cells with the first agent. In some embodiments, the exogenous nucleic acid is introduced by transduction or transfection.

In some embodiments, the chimeric polypeptide is a chimeric antigen receptor (CAR). In some embodiments, the chimeric antigen receptor (CAR) comprises and antigen binding region, a transmembrane region, and an intracellular signaling region. In some embodiments, the intracellular signaling region comprises one or more signaling domain. In some embodiments, the intracellular signaling domain comprises a signaling domain from CD27, CD28, 4-1BB, ICOS, OX40, DAP10, DAP12, CD134, CD3-zeta or fragment or combination thereof. In some embodiments, the transmembrane region comprises a transmembrane region from CD8, CD28, or CTLA4.

In some embodiments, the antigen binding region comprises an antibody domain. In some embodiments, the antibody domain comprises a scFv or a Fab. In some embodiments, the antigen binding region specifically binds a tumor associated antigen.

In some embodiments, the chimeric polypeptide is a chimeric T cell receptor (TCR). In some embodiments, the chimeric TCR comprises an antigen binding region. In some embodiments, the chimeric TCR further comprises a transmembrane region. In some embodiments, the chimeric TCR further comprises an intracellular signaling region. In some embodiments, the chimeric TCR comprises a TCR α polypeptide and a TCR β polypeptide. In some embodiments, chimeric TCR comprises a TCR γ polypeptide and a TCR δ polypeptide.

In some embodiments, the plurality of T cells comprises one or more T cells from a human subject. In some embodiments, the one or more T cell are removed from the human subject via apheresis. In some embodiments, the plurality of T cells comprises one or more T cell from a human subject that is healthy (e.g., a subject that does not have or has not been diagnosed with a specified disease or condition, e.g., a cancer). In some embodiments, the plurality of T cells comprises one or more T cells from a mammalian (e.g., human) subject having or diagnosed with a disease or condition (e.g., diagnosed with a specified disease or condition, e.g., cancer). In some embodiments, the disease is a cancer. In some embodiments, the cancer is a solid tumor or hematological cancer. In some embodiments, the cancer is selected from the group consisting of leukemia, lymphoma, myeloma, prostate, lung, renal, stomach, colon, ovarian, bladder, breast, cervical, esophageal, testicular, liver, pancreatic, rectal, thyroid, uterine, skin, muscle, cartilage, bone, endothelial, epithelial, dermal, basal, retinal, skin, or brain.

In some embodiments, the plurality of T cells comprises one or more autologous T cell. In some embodiments, the plurality of T cells comprises one or more allogeneic T cell.

In some embodiments, the number of cells in the first T cell population is at least 1, 2, 3, 4, 5, 6, 7, 8, 9, 10, 50, 100, or 1000 fold greater than the number of cells in the plurality of T cells prior to be contacted with the first agent.

In some embodiments, the agent that specifically binds CD3 (e.g., CD3ε) comprises an antibody domain (e.g., an anti-CD3 antibody (e.g., an anti-CD3ε antibody)).

In some embodiments, the agent that specifically binds CD3 specifically binds CD3ε.

In some embodiments, the first agent, upon binding to the TCRβV region, results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, the first agent, upon binding to the TCRβV region, results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., T effector memory (TEM) cells, e.g., TEM cells expressing CD45RA (TEMRA) cells.

In some embodiments, expansion of a population of memory effector T cells, e.g., TEM cells, e.g., TEMRA cells, in the first population of T cells a is increased compared to expansion of a similar population of cells with an antibody that binds to a CD3 molecule.

In some embodiments, the population of expanded T effector memory cells comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7−, CD95+ T cells, optionally wherein the T cells comprise CD3+, CD4+ or CD8+ T cells.

In some embodiments, binding of the first agent to the TCRβV region results in one, two, three, four, five, six, seven, eight, nine, ten or more (e.g., all) of the following: (i) reduced level, e.g., expression level, and/or activity of IL-10; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, binding of the first agent to the TCRβV region results in a reduction of at least 2, 5, 10, 20, 50, 100, or 200 fold, or at least 2-200 fold (e.g., 5-150, 10-100, 20-50 fold) in the expression level and or activity of IL-1β as measured by an assay described herein.

In some embodiments, binding of the first agent to the TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 as measured by an assay described herein.

In some embodiments, binding of the first agent to the TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα as measured by an assay described herein.

In some embodiments, binding of the first agent to the TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay described herein.

Provided herein are, inter alia, recombinant nucleic acids encoding a chimeric antigen receptor (CAR), wherein the CAR comprises (a) an antigen binding domain, wherein the antigen binding domain does not contain a T cell receptor α (TCRα) variable region or a T cell receptor β (TCRβ) variable region; (b) a transmembrane domain; and (c) an intracellular signaling domain comprising a TCRβ constant region intracellular domain; wherein the intracellular signaling domain does not contain a functional CD3 signaling domain. Also provided herein are, inter alia, recombinant nucleic acid encoding a chimeric antigen receptor (CAR), wherein the CAR comprises (a) an antigen binding domain, wherein the antigen binding domain is a single chain variable fragment (scFv) or a single domain antibody; (b) a transmembrane domain; and (c) an intracellular signaling domain comprising a TCRβ intracellular domain; wherein the intracellular signaling domain does not contain a functional CD3ζ signaling domain.

In some embodiments, the chimeric antigen receptor (CAR) does not contain a T cell receptor α (TCRα) variable region or a T cell receptor β (TCRβ) variable region.

In some embodiments, the antigen binding domain, transmembrane domain, and intracellular signaling domain are operatively linked.

In some embodiments, the CAR further comprises a TCRβ constant domain. In some embodiments, the TCRβ constant domain is a TCRβ 1 constant domain. In some embodiments, the TCRβ constant domain is a TCRβ 2 constant domain. In some embodiments, the CAR comprises a TCRβ constant domain 1 at least 90%, 91%, 92%, 93%, 94%, 95%, 96%, 97%, 98%, 99%, or 100% homologous to SEQ ID NO: 296.

In some embodiments, the CAR comprises a TCRβ constant domain 1 at least 90%, 91%, 92%, 93%, 94%, 95%, 96%, 97%, 98%, 99%, or 100% homologous to SEQ ID NO: 297.

In some embodiments, the CAR comprises a TCRβ constant domain 2 at least 90%, 91%, 92%, 93%, 94%, 95%, 96%, 97%, 98%, 99%, or 100% homologous to SEQ ID NO: 300.

In some embodiments, the CAR comprises a TCRβ constant domain 2 at least 90%, 91%, 92%, 93%, 94%, 95%, 96%, 97%, 98%, 99%, or 100% homologous to SEQ ID NO: 301.

In some embodiments, the transmembrane domain comprises a transmembrane domain of a protein selected from the group consisting of a T cell receptor β chain, T cell receptor α chain, CD28, CD3ε, CD45, CD4, CD5, CD8, CD9, CD16, CD22, CD33, CD37, CD64, CD80, CD86, CD134, CD137 or CD154.

In some embodiments, the transmembrane domain comprises a TCRβ constant 1 domain. In some embodiments, the transmembrane domain comprises a TCRβ constant 2 domain.

In some embodiments, the transmembrane domain comprises a nucleic acid encoding an amino acid sequence at least 90%, 91%, 92%, 93%, 94%, 95%, 96%, 97%, 98%, 99%, or 100% homologous to SEQ ID NO: 302.

In some embodiments, the transmembrane domain comprises a nucleic acid encoding an amino acid sequence at least 90%, 91%, 92%, 93%, 94%, 95%, 96%, 97%, 98%, 99%, or 100% homologous to SEQ ID NO: 298.

In some embodiments, the antigen binding domain is connected to the transmembrane domain by a linker. In some embodiments, the linker comprises or consists of glycine and serine.

In some embodiments, the TCRβ constant intracellular domain comprises a TCRβ constant 1 intracellular domain. In some embodiments, the TCRβ constant intracellular domain comprises a TCRβ constant 2 intracellular domain.

In some embodiments, the TCRβ intracellular domain comprises nucleic acid encoding an amino acid sequence at least 90%, 91%, 92%, 93%, 94%, 95%, 96%, 97%, 98%, 99%, or 100% homologous to SEQ ID NO: 299.

In some embodiments, the TCRβ intracellular domain comprises nucleic acid encoding an amino acid sequence at least 90%, 91%, 92%, 93%, 94%, 95%, 96%, 97%, 98%, 99%, or 100% homologous to SEQ ID NO: 303.

In some embodiments, the intracellular signaling domain further comprises a costimulatory signaling domain. In some embodiments, the intracellular signaling domain comprises from N to C terminus one or more costimulatory signaling domains and a TCRβ constant region intracellular domain.

In some embodiments, the costimulatory signaling domain comprises one or more functional signaling domain of one or more protein selected from the group consisting of 4-1BB (CD137), OX40, CD2, CD7, CD27, CD28, CD30, CD40, CDS, ICAM-1, B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, MEW class I molecules, TNF receptor proteins, Immunoglobulin-like proteins, cytokine receptors, integrins, signaling lymphocytic activation molecules (SLAM proteins), activating NK cell receptors, BTLA, and a Toll ligand receptor.

In some embodiments, the antigen binding domain is a human or humanized single chain variable fragment (scFv) or single domain antibody (sdAb). In some embodiments, the antigen binding domain comprises a single chain variable fragment (scFv). In some embodiments, the antigen binding domain comprises a single domain antibody (sdAb).

In some embodiments, the antigen binding domain binds to a tumor associated antigen.

In some embodiments, the encoded chimeric antigen receptor (CAR) is expressed in frame and as a single polypeptide chain.

Provided herein are, inter alia, vectors comprising the nucleic acid molecule described herein. In some embodiments, the vector is a DNA vector, a RNA vector, a plasmid, a lentivirus vector, an adenoviral vector, or a retrovirus vector.

Provided herein are, inter alia, methods of making an immune effector cell, comprising transducing the immune effector cell with a vector described herein. In some embodiments, the immune effector cell is a T cell or an NK cell. In some embodiments, the immune effector cell is an autologous or allogenic immune effector cell.

Provided herein are, inter alia, immune effector cells comprising the nucleic acid molecule described herein.

In some embodiments, the immune effector cell is made by a method described herein. In some embodiments, the immune effector cell is a T cell or an NK cell. In some embodiments, the immune effector cell is an autologous or allogenic immune effector cell.

In some embodiments, upon binding of the antigen binding domain to a cognate antigen expressed by a cell, the level of expression (e.g., release) of one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by the immune effector cell is lower relative to the level of expression (e.g., release) of the one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by an immune effector cell comprising a nucleic acid encoding a CAR comprising a CD3ζ intracellular signaling domain.

In some embodiments, upon binding of the antigen binding domain to a cognate antigen expressed by a cell, the level of expression (e.g., release) of one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by the immune effector cell is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by an immune effector cell comprising a nucleic acid encoding a CAR comprising a CD3ζ intracellular signaling domain.

In some embodiments, upon binding of the antigen binding domain to a cognate antigen expressed by a cell in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a CAR comprising a CD3ζ intracellular signaling domain.

In some embodiments, upon binding of the antigen binding domain to a cognate antigen expressed by a cell in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a CAR comprising a CD3ζ intracellular signaling domain.

In some embodiments, upon binding of the antigen binding domain to a cognate antigen expressed by a cell in vitro, in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is lower relative to the level of expression (e.g., release) of the one or more proinflammatory cytokines (e.g., IL-6, IFNγ, TNFα, IL-6, IL-10, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a CAR comprising a CD3ζ intracellular signaling domain.

In some embodiments, upon binding of the antigen binding domain to a cognate antigen expressed by a cell in vitro, in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP10, and GM-CSF) by one or more (e.g., a population) antigen presenting cell in the presence of an immune effector cell comprising a nucleic acid encoding a CAR comprising a CD3ζ intracellular signaling domain.

Provided herein are, inter alia, pharmaceutical compositions comprising the immune effector cell described herein.

Provided herein are, inter alia, polypeptides encoded by the recombinant nucleic acid described herein.

Provided herein are, inter alia, methods of generating a population of RNA-engineered cells comprising introducing an in vitro transcribed RNA or synthetic RNA into a cell, wherein the RNA comprises the nucleic acid molecule described herein.

Provided herein are, inter alia, chimeric antigen receptors (CARs) comprising: (a) an antigen binding domain, wherein the antigen binding domain does not contain a T cell receptor α (TCRα) variable region or a T cell receptor (TCRβ) variable region; (b) a transmembrane domain; and (c) an intracellular signaling domain comprising a TCRβ constant region intracellular domain; wherein the intracellular signaling domain does not contain a functional CD3ζ intracellular signaling domain. Provided herein are also, inter alia, chimeric antigen receptors (CARs) comprising: (a) an antigen binding domain, wherein the antigen binding domain is a single chain variable fragment (scFv) or a single domain antibody; (b) a transmembrane domain; and (c) an intracellular signaling domain comprising a TCRβ intracellular domain; wherein the intracellular signaling domain does not contain a functional CD3ζ intracellular signaling domain.

In some embodiments, the CAR does not comprise a T cell receptor α (TCRα) variable region or a T cell receptor β (TCRβ) variable region.

In some embodiments, the antigen binding domain, transmembrane domain, and intracellular signaling domain are operatively linked. In some embodiments, the transmembrane domain comprises a transmembrane domain of a protein selected from the group consisting of a T cell receptor β chain, T cell receptor α chain, CD28, CD3ε, CD45, CD4, CD5, CD8, CD9, CD16, CD22, CD33, CD37, CD64, CD80, CD86, CD134, CD137 and CD154.

In some embodiments, the transmembrane domain comprises a TCRβ transmembrane domain. In some embodiments, the transmembrane domain comprises a TCRβ 1 transmembrane domain. In some embodiments, the transmembrane domain comprises a TCRβ 2 transmembrane domain.

In some embodiments, the transmembrane domain comprises a nucleic acid encoding an amino acid sequence at least 90%, 91%, 92%, 93%, 94%, 95%, 96%, 97%, 98%, 99%, or 100% homologous to SEQ ID NO: 298.

In some embodiments, the transmembrane domain comprises a nucleic acid encoding an amino acid sequence at least 90%, 91%, 92%, 93%, 94%, 95%, 96%, 97%, 98%, 99%, or 100% homologous to SEQ ID NO: 302.

In some embodiments, the antigen binding domain is connected to the transmembrane domain by a linker. In some embodiments, the linker comprises glycine and serine.

In some embodiments, the TCRβ constant intracellular domain comprises a TCRβ constant 1 intracellular domain. In some embodiments, the TCRβ constant intracellular domain comprises a TCRβ constant 2 intracellular domain.

In some embodiments, the intracellular signaling domain comprises nucleic acid encoding an amino acid sequence at least 90%, 91%, 92%, 93%, 94%, 95%, 96%, 97%, 98%, 99%, or 100% homologous to SEQ ID NO: 299.

In some embodiments, the intracellular signaling domain comprises nucleic acid encoding an amino acid sequence at least 90%, 91%, 92%, 93%, 94%, 95%, 96%, 97%, 98%, 99%, or 100% homologous to SEQ ID NO: 303.

In some embodiments, the intracellular signaling domain further comprises a costimulatory signaling domain. In some embodiments, the intracellular signaling domain comprises from N to C terminus one or more costimulatory signaling domains and a TCRβ constant region intracellular domain. In some embodiments, the costimulatory signaling domain comprises one or more functional signaling domain of one or more protein selected from the group consisting of 4-1BB (CD137), OX40, CD2, CD7, CD27, CD28, CD30, CD40, CDS, ICAM-1, B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, MHC class I molecules, TNF receptor proteins, Immunoglobulin-like proteins, cytokine receptors, integrins, signaling lymphocytic activation molecules (SLAM proteins), activating NK cell receptors, BTLA, and a Toll ligand receptor.

In some embodiments, the antigen binding domain is a human or humanized single chain variable fragment (scFv) and single domain antibody.

In some embodiments, the antigen binding domain comprises a single chain variable fragment (scFv). In some embodiments, the antigen binding domain comprises a single domain antibody (sdAb).

In some embodiments, the antigen binding domain binds to a tumor associated antigen.

In some embodiments, the CAR is manufactured by a method described herein.

Provided herein are, inter alia, methods of treating a cancer in a subject, the method comprising: administering to the subject a cell (e.g., a population of cells, e.g., a population of immune effector cells), expressing a chimeric antigen receptor (CAR) described herein.

In some embodiments, the chimeric antigen receptor (CAR) is encoded by a nucleic acid molecule described herein.

Provided herein are, inter alia, methods of preventing cytokine release syndrome (CRS) in a subject having a cancer (e.g., CRS associated with or induced by administration of a chimeric antigen receptor (CAR) cell therapy), the method comprising administering to the subject a cell (e.g., a population of cells, e.g., a population of immune effector cells), expressing a chimeric antigen receptor (CAR) described herein.

In some embodiments, the chimeric antigen receptor (CAR) is encoded by a nucleic acid molecule described herein.

In some embodiments, the subject does not exhibit one or more symptom of CRS (e.g., as described herein) within 1, 2, 3, 4, 5, 6, 7, 10, 14, 21, or 30 days of administration of the cell (e.g., a population of cells, e.g., a population of immune effector cells). In some embodiments, the subject does not exhibit one or more symptom grade 4 or grade 5 CRS (e.g., as described herein). In some embodiments, the subject does not exhibit any symptom grade 4 or grade 5 CRS (e.g., as described herein). In some embodiments, the level of one or more protein selected from the group consisting of IL-6, IL-1β, IL-8, IL-10, IFNγ, TNFα, sIL2Rα, sgp130, sIL6R, MCP1, MIP1α, MIP1β, and GM-CSF, in the serum of the subject post administration (e.g., 1 hour, 6 hours, 12 hours, 1 day, 2 days, 3 days, 4 days, 5, days, 6 days, 7 days, 10 days, 14 days, 21 days, 30 days) of the cell (e.g., population of cells, e.g., population of immune effector cells) is within ±20%, ±15%, ±10%, ±9%, ±8%, ±7%, ±6%, ±5%, ±4%, ±3%, ±2% or ±1% of the level of the one or more protein in the serum of the subject prior to administration (e.g., 10 minutes, 30 minutes, 1 hour, 6 hours, 12 hours, 24 hours, 48 hours, 72 hours) of the cell (e.g., population of cells, e.g., population of immune effector cells).

In some embodiments, the methods further comprise selecting the subject for administration of the cell (e.g., population of cells, e.g., population of immune effector cells) based on a determination of one or more of the following: the subject's risk of developing CRS, the subject's risk of developing CRS if administered a cell expressing a CAR comprising a CD3 signaling domain, the subject's diagnosis of CRS, the subject's diagnosis of CRS associated with or induced by administration of a cell expressing a CAR comprising a CD3ζ signaling domain.

In some embodiments, the subject is selected for administration if the subject is at risk of developing CRS, if the subject is at risk of developing CRS if administered a CAR comprising a cell expressing a CAR CD3ζ signaling domain, if the subject has been diagnosed with CRS, if the subject has been diagnosed with CRS associated with or induced by administration of a cell expressing a CAR comprising a CD3ζ signaling domain.

In some embodiments, the cell (e.g., population of cells, e.g., population of immune effector cells) is a cell described herein. In some embodiments, the cell (e.g., population of cells, e.g., population of immune effector cells) is a T cell or NK cell. In some embodiments, the cell (e.g., population of cells, e.g., population of immune effector cells) is an autologous or allogenic immune effector cell.

In some embodiments, the subject is a mammal, e.g., a human.

In some embodiments, the cell (e.g., population of cells, e.g., population of immune effector cells) is administered in combination is a further therapeutic agent.

In some embodiments, the cancer is a solid cancer or hematological cancer. In some embodiments, the cancer is a solid cancer. In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer. In some embodiments, the hematologic cancer is a leukemia, lymphoma, or myeloma. In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CIVIL), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

Provided herein are, inter alia, recombinant T cell receptors (TCRs) comprising: (a) a TCRα chain comprising i) an immunoglobulin variable heavy domain, ii) a TCRα transmembrane domain, and iii) an intracellular signaling domain comprising optionally a TCRα intracellular domain; (b) a TCRβ chain comprising i) an immunoglobulin variable light domain, ii) a TCRβ transmembrane domain, and iii) an intracellular signaling domain comprising a TCRβ intracellular domain; wherein the immunoglobulin variable heavy domain and the immunoglobulin variable light domain form an antigen binding domain; wherein the recombinant TCR does not contain a functional CD3ζ intracellular signaling domain; and wherein the recombinant TCR does not comprise a T cell receptor α (TCRα) variable region or a T cell receptor β (TCRβ) variable region.

Provided herein are also, inter alia, recombinant T cell receptor (TCR) comprising: (a) a TCRα chain comprising i) an immunoglobulin variable light domain, ii) a TCRα transmembrane domain, and iii) an intracellular signaling domain comprising optionally a TCRα intracellular domain; (b) a TCRβ chain comprising i) an immunoglobulin variable heavy domain, ii) a TCRβ transmembrane domain, and iii) an intracellular signaling domain comprising a TCRβ intracellular domain; wherein the immunoglobulin variable heavy domain and the immunoglobulin variable light domain form an antigen binding domain; wherein the recombinant TCR does not contain a functional CD3ζ intracellular signaling domain; and wherein the recombinant TCR does not comprise a T cell receptor α (TCRα) variable region or a T cell receptor β (TCRβ) variable region.

In some embodiments, the TCRα chain further comprises a TCRα constant domain.

In some embodiments, the TCRα chain further comprises a TCRα constant domain at least 90%, 91%, 92%, 93%, 94%, 95%, 96%, 97%, 98%, 99%, or 100% homologous to SEQ ID NO: 293.

In some embodiments, the TCRβ chain further comprises a TCRβ constant domain.

In some embodiments, the TCRβ constant domain comprises a TCRβ constant domain 1.

In some embodiments, the TCRβ constant domain comprises a TCRβ constant domain 2.

In some embodiments, the TCRβ chain comprises a TCRβ constant domain 1 at least 90%, 91%, 92%, 93%, 94%, 95%, 96%, 97%, 98%, 99%, or 100% homologous to SEQ ID NO: 296.

In some embodiments, the TCRβ chain comprises a TCRβ constant domain 1 at least 90%, 91%, 92%, 93%, 94%, 95%, 96%, 97%, 98%, 99%, or 100% homologous to SEQ ID NO: 297.

In some embodiments, the TCRβ chain further comprises a TCRβ constant domain 2 at least 90%, 91%, 92%, 93%, 94%, 95%, 96%, 97%, 98%, 99%, or 100% homologous to SEQ ID NO: 300.

In some embodiments, the TCRβ chain comprises a TCRβ constant domain 2 at least 90%, 91%, 92%, 93%, 94%, 95%, 96%, 97%, 98%, 99%, or 100% homologous to SEQ ID NO: 301.

In some embodiments, the TCRβ transmembrane domain comprises a nucleic acid encoding an amino acid sequence at least 90%, 91%, 92%, 93%, 94%, 95%, 96%, 97%, 98%, 99%, or 100% homologous to SEQ ID NO: 302.

In some embodiments, the TCRβ transmembrane domain comprises a nucleic acid encoding an amino acid sequence at least 90%, 91%, 92%, 93%, 94%, 95%, 96%, 97%, 98%, 99%, or 100% homologous to SEQ ID NO: 298.

In some embodiments, the TCRα transmembrane domain comprises a nucleic acid encoding an amino acid sequence at least 90%, 91%, 92%, 93%, 94%, 95%, 96%, 97%, 98%, 99%, or 100% homologous to SEQ ID NO: 294.

In some embodiments, the antigen binding domain is connected to the transmembrane domain by a linker. In some embodiments, the linked comprises or consists of glycine and serine.

In some embodiments, the TCRβ intracellular domain comprises a TCRβ 1 intracellular domain. In some embodiments, the TCRβ intracellular domain comprises a TCRβ 2 intracellular domain.

In some embodiments, the TCRβ intracellular domain comprises nucleic acid encoding an amino acid sequence at least 90%, 91%, 92%, 93%, 94%, 95%, 96%, 97%, 98%, 99%, or 100% homologous to SEQ ID NO: 299.

In some embodiments, the TCRβ intracellular domain comprises nucleic acid encoding an amino acid sequence at least 90%, 91%, 92%, 93%, 94%, 95%, 96%, 97%, 98%, 99%, or 100% homologous to SEQ ID NO: 303.

In some embodiments, the TCRα intracellular domain comprises nucleic acid encoding an amino acid sequence at least 90%, 91%, 92%, 93%, 94%, 95%, 96%, 97%, 98%, 99%, or 100% homologous to SEQ ID NO: 295.

In some embodiments, the TCRα intracellular signaling domain further comprises a costimulatory signaling domain. In some embodiments, the TCRβ intracellular signaling domain further comprises a costimulatory signaling domain.

In some embodiments, the costimulatory signaling domain comprises one or more functional signaling domain of one or more protein selected from the group consisting of 4-1BB (CD137), OX40, CD2, CD7, CD27, CD28, CD30, CD40, CDS, ICAM-1, B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, MHC class I molecules, TNF receptor proteins, Immunoglobulin-like proteins, cytokine receptors, integrins, signaling lymphocytic activation molecules (SLAM proteins), activating NK cell receptors, BTLA, and a Toll ligand receptor.

In some embodiments, i) the immunoglobulin variable heavy domain and the immunoglobulin variable light domain are humanized; or ii) the immunoglobulin variable heavy domain and the immunoglobulin variable light domain are human.

In some embodiments, the antigen binding domain binds to a tumor associated antigen.

In some embodiments, the recombinant T cell receptor (TCR) is manufactured by a method described herein.

Provided herein are, inter alia, recombinant nucleic acids encoding a recombinant TCR described herein.

Provided herein are, inter alia, polypeptides encoding a recombinant TCR described herein encoded by a nucleic acid described herein.

Provided herein are, inter alia, vectors comprising the nucleic acid molecule encoding a recombinant TCR described herein. In some embodiments, the vector is a DNA vector, a RNA vector, a plasmid, a lentivirus vector, an adenoviral vector, or a retrovirus vector.

Provided herein are, inter alia, methods of making an immune effector cell, comprising transducing the immune effector cell with the vector described herein. In some embodiments, the immune effector cell is a T cell or an NK cell. In some embodiments, the immune effector cell is an autologous or allogenic immune effector cell.

Provided herein are, inter alia, immune effector cells comprising the nucleic acid molecule described herein encoding a recombinant TCR described herein.

In some embodiments, the immune effector cell is made by a method described herein. In some embodiments, the immune effector cell is a T cell or an NK cell. In some embodiments, the immune effector cell is an autologous or allogenic immune effector cell. In some embodiments, upon binding of the antigen binding domain to a cognate antigen expressed by a cell, the level of expression (e.g., release) of one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by the immune effector cell is lower relative to the level of expression (e.g., release) of the one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by an immune effector cell comprising a nucleic acid encoding a TCR comprising a CD3ζ intracellular signaling domain.

In some embodiments, upon binding of the antigen binding domain to a cognate antigen expressed by a cell, the level of expression (e.g., release) of one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by the immune effector cell is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by an immune effector cell comprising a nucleic acid encoding a TCR comprising a CD3ζ intracellular signaling domain.

In some embodiments, upon binding of the antigen binding domain to a cognate antigen expressed by a cell in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

In some embodiments, upon binding of the antigen binding domain to a cognate antigen expressed by a cell in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

In some embodiments, upon binding of the antigen binding domain to a cognate antigen expressed by a cell in vitro, in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

In some embodiments, upon binding of the antigen binding domain to a cognate antigen expressed by a cell in vitro, in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

Provided herein are, inter alio, pharmaceutical compositions comprising the immune effector cell described herein.

Provided herein are, inter alio, methods of treating a cancer in a subject, the method comprising: administering to the subject a cell (e.g., a population of cells, e.g., a population of immune effector cells), expressing a TCR described herein.

In some embodiments, the recombinant T cell receptor (TCR) is encoded by a nucleic acid molecule described herein.

Provided herein are, inter alio, methods of preventing cytokine release syndrome (CRS) in a subject having a cancer (e.g., CRS associated with or induced by administration of a recombinant T cell receptor (TCR) cell therapy), the method comprising administering to the subject a cell (e.g., a population of cells, e.g., a population of immune effector cells), expressing a recombinant T cell receptor (TCR) described herein.

In some embodiments, the recombinant T cell receptor (TCR) is encoded by a nucleic acid molecule described herein.

In some embodiments, the subject does not exhibit one or more symptom of CRS (e.g., as described herein) within 1, 2, 3, 4, 5, 6, 7, 10, 14, 21, or 30 days of administration of the cell (e.g., a population of cells, e.g., a population of immune effector cells). In some embodiments, the subject does not exhibit one or more symptom grade 4 or grade 5 CRS (e.g., as described herein). In some embodiments, the subject does not exhibit any symptom grade 4 or grade 5 CRS (e.g., as described herein). In some embodiments, the level of one or more protein selected from the group consisting of IL-6, IL-10, IL-8, IL-10, IFNγ, TNFα, sIL2Rα, sgp130, sIL6R, MCP1, MIP1α, MIP1β, and GM-CSF, in the serum of the subject post administration (e.g., 1 hour, 6 hours, 12 hours, 1 day, 2 days, 3 days, 4 days, 5, days, 6 days, 7 days, 10 days, 14 days, 21 days, 30 days) of the cell (e.g., population of cells, e.g., population of immune effector cells) is within ±20%, ±15%, ±10%, ±9%, ±8%, ±7%, ±6%, ±5%, ±4%, ±3%, ±2% or ±1% of the level of the one or more protein in the serum of the subject prior to administration (e.g., 10 minutes, 30 minutes, 1 hour, 6 hours, 12 hours, 24 hours, 48 hours, 72 hours) of the cell (e.g., population of cells, e.g., population of immune effector cells).

In some embodiments, the method further comprises selecting the subject for administration of the cell (e.g., population of cells, e.g., population of immune effector cells) based on a determination of one or more of the following: the subject's risk of developing CRS, the subject's risk of developing CRS if administered a cell expressing a recombinant TCR comprising a CD3ζ signaling domain, the subject's diagnosis of CRS, the subject's diagnosis of CRS associated with or induced by administration of a cell expressing a recombinant TCR comprising a CD3ζ signaling domain.

In some embodiments, the subject is selected for administration if the subject is at risk of developing CRS, if the subject is at risk of developing CRS if administered a cell expressing a recombinant TCR comprising a CD3ζ signaling domain, if the subject has been diagnosed with CRS, if the subject has been diagnosed with CRS associated with or induced by administration of a cell expressing a recombinant TCR comprising a CD3ζ signaling domain.

In some embodiments, the cell (e.g., population of cells, e.g., population of immune effector cells) is a cell described herein. In some embodiments, the cell (e.g., population of cells, e.g., population of immune effector cells) is a T cell or NK cell. In some embodiments, the cell (e.g., population of cells, e.g., population of immune effector cells) is an autologous or allogenic immune effector cell.

In some embodiments, the subject is a mammal, e.g., a human.

In some embodiments, the cell (e.g., population of cells, e.g., population of immune effector cells) is administered in combination is a further therapeutic agent.

In some embodiments, the cancer is a solid cancer or hematological cancer. In some embodiments, the cancer is a solid cancer. In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer. In some embodiments, the cancer is a hematologic cancer. In some embodiments, the hematologic cancer is a leukemia, lymphoma, or myeloma. In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CIVIL), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

Provided herein are, inter alia, recombinant T cell receptors (TCRs) comprising: (a) a TCRα chain comprising i) an antigen binding domain (e.g., a scFv), ii) a TCRα variable domain, iii) a TCRα constant domain, iv) a TCRα transmembrane domain, and iii) an intracellular signaling domain comprising optionally a TCRα intracellular domain; (b) a TCRβ chain comprising i) an TCRβ variable domain, ii) a TCRβ constant domain, iii) a TCRβ transmembrane domain, and iv) an intracellular signaling domain comprising a TCRβ intracellular domain; and wherein the recombinant TCR does not contain a functional CD3ζ intracellular signaling domain. Provided herein are, inter alia, recombinant T cell receptors (TCRs) comprising:

(a) a TCRα chain comprising i) a TCRα variable domain, ii) a TCRα constant domain, iii) a TCRα transmembrane domain, and iv) an intracellular signaling domain comprising optionally a TCRα intracellular domain; (b) a TCRβ chain comprising i) an antigen binding domain (e.g., a scFv), ii) an TCRβ variable domain, iii) a TCRβ constant domain, iii) a TCRβ transmembrane domain, and iv) an intracellular signaling domain comprising a TCRβ intracellular domain; and wherein the recombinant TCR does not contain a functional CD3ζ intracellular signaling domain.

In some embodiments, the TCRα constant domain is at least 90%, 91%, 92%, 93%, 94%, 95%, 96%, 97%, 98%, 99%, or 100% homologous to SEQ ID NO: 293.

In some embodiments, the TCRβ constant domain comprises a TCRβ constant domain 1.

In some embodiments, the TCRβ constant domain comprises a TCRβ constant domain 2.

In some embodiments, the TCRβ constant domain 1 is at least 90%, 91%, 92%, 93%, 94%, 95%, 96%, 97%, 98%, 99%, or 100% homologous to SEQ ID NO: 296.

In some embodiments, the TCRβ constant domain 1 is at least 90%, 91%, 92%, 93%, 94%, 95%, 96%, 97%, 98%, 99%, or 100% homologous to SEQ ID NO: 297.

In some embodiments, the TCRβ constant domain 2 is at least 90%, 91%, 92%, 93%, 94%, 95%, 96%, 97%, 98%, 99%, or 100% homologous to SEQ ID NO: 300.

In some embodiments, the TCRβ constant domain 2 is at least 90%, 91%, 92%, 93%, 94%, 95%, 96%, 97%, 98%, 99%, or 100% homologous to SEQ ID NO: 301.

In some embodiments, the TCRβ transmembrane domain comprises a nucleic acid encoding an amino acid sequence at least 90%, 91%, 92%, 93%, 94%, 95%, 96%, 97%, 98%, 99%, or 100% homologous to SEQ ID NO: 302.

In some embodiments, the TCRβ transmembrane domain comprises a nucleic acid encoding an amino acid sequence at least 90%, 91%, 92%, 93%, 94%, 95%, 96%, 97%, 98%, 99%, or 100% homologous to SEQ ID NO: 298.

In some embodiments, the TCRα transmembrane domain comprises a nucleic acid encoding an amino acid sequence at least 90%, 91%, 92%, 93%, 94%, 95%, 96%, 97%, 98%, 99%, or 100% homologous to SEQ ID NO: 294.

In some embodiments, the antigen binding domain is connected to the transmembrane domain by a linker. In some embodiments, the linked comprises or consists of glycine and serine.

In some embodiments, the TCRβ intracellular domain comprises a TCRβ 1 intracellular domain. In some embodiments, the TCRβ intracellular domain comprises a TCRβ 2 intracellular domain.

In some embodiments, the TCRβ intracellular domain comprises nucleic acid encoding an amino acid sequence at least 90%, 91%, 92%, 93%, 94%, 95%, 96%, 97%, 98%, 99%, or 100% homologous to SEQ ID NO: 299.

In some embodiments, the TCRβ intracellular domain comprises nucleic acid encoding an amino acid sequence at least 90%, 91%, 92%, 93%, 94%, 95%, 96%, 97%, 98%, 99%, or 100% homologous to SEQ ID NO: 303.

In some embodiments, the TCRα intracellular domain comprises nucleic acid encoding an amino acid sequence at least 90%, 91%, 92%, 93%, 94%, 95%, 96%, 97%, 98%, 99%, or 100% homologous to SEQ ID NO: 295.

In some embodiments, the TCRα intracellular signaling domain further comprises a costimulatory signaling domain. In some embodiments, the TCRβ intracellular signaling domain further comprises a costimulatory signaling domain. In some embodiments, the costimulatory signaling domain comprises one or more functional signaling domain of one or more protein selected from the group consisting of 4-1BB (CD137), OX40, CD2, CD7, CD27, CD28, CD30, CD40, CDS, ICAM-1, B7-H3, ICOS (CD278), GITR, BAFFR, LIGHT, HVEM (LIGHTR), KIRDS2, SLA1VIF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, a ligand that specifically binds with CD83, MHC class I molecules, TNF receptor proteins, Immunoglobulin-like proteins, cytokine receptors, integrins, signaling lymphocytic activation molecules (SLAM proteins), activating NK cell receptors, BTLA, and a Toll ligand receptor.

In some embodiments, the antigen binding domain is a scFv, a single domain antibody, or a nanobody. In some embodiments, the antigen binding domain binds to a tumor associated antigen.

In some embodiments, the TCR is manufactured by a method described herein.

Provided herein are, inter alia, recombinant nucleic acids encoding a recombinant TCR described herein.

Provided herein are, inter alia, polypeptides encoded by the nucleic acid described herein.

Provided herein are, inter alia, vectors comprising the nucleic acid molecule described herein.

In some embodiments, the vector is a DNA vector, a RNA vector, a plasmid, a lentivirus vector, an adenoviral vector, or a retrovirus vector.

Provided herein are, inter alia, methods of making an immune effector cell, comprising transducing the immune effector cell with the vector described herein. In some embodiments, the immune effector cell is a T cell or an NK cell. In some embodiments, the immune effector cell is an autologous or allogenic immune effector cell.

Provided herein are, inter alia, immune effector cells comprising the nucleic acid molecule described herein. In some embodiments, the immune effector cell is made by a method described herein. In some embodiments, the immune effector cell is a T cell or an NK cell. In some embodiments, the immune effector cell is an autologous or allogenic immune effector cell.

In some embodiments, upon binding of the antigen binding domain to a cognate antigen expressed by a cell, the level of expression (e.g., release) of one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by the immune effector cell is lower relative to the level of expression (e.g., release) of the one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by an immune effector cell comprising a nucleic acid encoding a TCR comprising a CD3ζ intracellular signaling domain.

In some embodiments, upon binding of the antigen binding domain to a cognate antigen expressed by a cell, the level of expression (e.g., release) of one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by the immune effector cell is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IFNγ, TNFα, IL-6, IL-1β, IL-8, IL-10, sIL2Rα, sgp130, sIL6R, MCP1, MIP1α, MIP1β, and GM-CSF) by an immune effector cell comprising a nucleic acid encoding a TCR comprising a CD3ζ intracellular signaling domain.

In some embodiments, upon binding of the antigen binding domain to a cognate antigen expressed by a cell in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

In some embodiments, upon binding of the antigen binding domain to a cognate antigen expressed by a cell in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

In some embodiments, upon binding of the antigen binding domain to a cognate antigen expressed by a cell in vitro, in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

In some embodiments, upon binding of the antigen binding domain to a cognate antigen expressed by a cell in vitro, in the presence of one or more an antigen presenting cell, the level of expression (e.g., release) of one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell (e.g., dendritic cell or macrophage) is at least 1%, 2%, 3%, 4%, 5%, 6%, 7%, 8%, 9%, 10%, 15%, 20%, 25%, 30%, 40%, 50%, 60%, 70%, 80%, 90%, 95%, 96%, 97%, 98%, or 99% lower relative to the level of expression (e.g., release) of the one or more proinflammatory cytokines (e.g., IL-6, IFNγ, TNFα, IL-6, IL-1β, IL-8, IL-10, sIL2Rα, sgp130, sIL6R, MCP1, MIP1α, MIP1β, and GM-CSF) by one or more (e.g., a population) antigen presenting cell in the presence of an immune effector cell comprising a nucleic acid encoding a TCR comprising a CD3ζ intracellular signaling domain.

Provided herein are, inter alio, pharmaceutical compositions comprising the immune effector cell described herein.

Provided herein are, inter alio, methods of treating a cancer in a subject, the method comprising: administering to the subject a cell (e.g., a population of cells, e.g., a population of immune effector cells), expressing a TCR described herein. In some embodiments, the recombinant T cell receptor (TCR) is encoded by a nucleic acid molecule described herein.

Provided herein are, inter alio, methods of preventing cytokine release syndrome (CRS) in a subject having a cancer (e.g., CRS associated with or induced by administration of a recombinant T cell receptor (TCR) cell therapy), the method comprising administering to the subject a cell (e.g., a population of cells, e.g., a population of immune effector cells), expressing a recombinant T cell receptor (TCR) described herein.

In some embodiments, the recombinant T cell receptor (TCR) is encoded by a nucleic acid molecule described herein.

In some embodiments, the subject does not exhibit one or more symptom of CRS (e.g., as described herein) within 1, 2, 3, 4, 5, 6, 7, 10, 14, 21, or 30 days of administration of the cell (e.g., a population of cells, e.g., a population of immune effector cells). In some embodiments, the subject does not exhibit one or more symptom grade 4 or grade 5 CRS (e.g., as described herein). In some embodiments, the subject does not exhibit any symptom grade 4 or grade 5 CRS (e.g., as described herein). In some embodiments, the level of one or more protein selected from the group consisting of IL-6, IL-10, IL-8, IL-10, IFNγ, TNFα, sIL2Rα, sgp130, sIL6R, MCP1, MIP1α, MIP1β, and GM-CSF, in the serum of the subject post administration (e.g., 1 hour, 6 hours, 12 hours, 1 day, 2 days, 3 days, 4 days, 5, days, 6 days, 7 days, 10 days, 14 days, 21 days, 30 days) of the cell (e.g., population of cells, e.g., population of immune effector cells) is within ±20%, ±15%, ±10%, ±9%, ±8%, ±7%, ±6%, ±5%, ±4%, ±3%, ±2% or ±1% of the level of the one or more protein in the serum of the subject prior to administration (e.g., 10 minutes, 30 minutes, 1 hour, 6 hours, 12 hours, 24 hours, 48 hours, 72 hours) of the cell (e.g., population of cells, e.g., population of immune effector cells).

In some embodiments, the method further comprises selecting the subject for administration of the cell (e.g., population of cells, e.g., population of immune effector cells) based on a determination of one or more of the following: the subject's risk of developing CRS, the subject's risk of developing CRS if administered a cell expressing a recombinant TCR comprising a CD3 signaling domain, the subject's diagnosis of CRS, the subject's diagnosis of CRS associated with or induced by administration of a cell expressing a recombinant TCR comprising a CD3ζ signaling domain.

In some embodiments, the subject is selected for administration if the subject is at risk of developing CRS, if the subject is at risk of developing CRS if administered a cell expressing a recombinant TCR comprising a CD3ζ signaling domain, if the subject has been diagnosed with CRS, if the subject has been diagnosed with CRS associated with or induced by administration of a cell expressing a recombinant TCR comprising a CD3ζ signaling domain.

In some embodiments, the cell (e.g., population of cells, e.g., population of immune effector cells) is a cell described herein. In some embodiments, the cell (e.g., population of cells, e.g., population of immune effector cells) is a T cell or NK cell. In some embodiments, the cell (e.g., population of cells, e.g., population of immune effector cells) is an autologous or allogenic immune effector cell.

In some embodiments, the subject is a mammal, e.g., a human.

In some embodiments, the cell (e.g., population of cells, e.g., population of immune effector cells) is administered in combination is a further therapeutic agent.

In some embodiments, the cancer is a solid cancer or hematological cancer. In some embodiments, the cancer is a solid cancer. In some embodiments, the solid cancer is a prostate cancer, lung cancer, renal cancer, stomach cancer, colon cancer, ovarian cancer, bladder cancer, breast cancer, cervical cancer, esophageal cancer, testicular cancer, liver cancer, pancreatic cancer, rectal cancer, thyroid cancer, uterine cancer, skin cancer, muscle cancer, cartilage cancer, bone cancer, endothelial cancer, epithelial cancer, dermal cancer, basal cancer, retinal cancer, skin cancer, or brain cancer. In some embodiments, the cancer is a hematologic cancer. In some embodiments, the hematologic cancer is a leukemia, lymphoma, or myeloma. In some embodiments, the hematologic cancer is B-cell acute lymphoid leukemia (B-ALL), T-cell acute lymphoid leukemia (T-ALL), acute lymphoblastic leukemia (ALL); chronic myelogenous leukemia (CIVIL), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell-follicular lymphoma, large cell-follicular lymphoma, malignant lymphoproliferative conditions, MALT lymphoma, mantle cell lymphoma, Marginal zone lymphoma, multiple myeloma, myelodysplasia, myelodysplastic syndrome, non-Hodgkin's lymphoma, plasmablastic lymphoma, plasmacytoid dendritic cell neoplasm, Waldenstrom macroglobulinemia, or preleukemia.

### BRIEF DESCRIPTION OF THE DRAWINGS

FIG. 1 depicts the phylogenetic tree of TCRβV gene family and subfamilies with corresponding antibodies mapped. Subfamily identities are as follows: Subfamily A: TCRβ V6; Subfamily B: TCRβ V10; Subfamily C: TCRβ V12; Subfamily D: TCRβ V5; Subfamily E: TCRβ V7; Subfamily F: TCRβ V11; Subfamily G: TCRβ V14; Subfamily H: TCRβ V16; Subfamily I:TCRβ V18; Subfamily J:TCRβ V9; Subfamily K: TCRβ V13; Subfamily L: TCRβ V4; Subfamily M:TCRβ V3; Subfamily N:TCRβ V2; Subfamily O:TCRβ V15; Subfamily P: TCRβ V30; Subfamily Q: TCRβ V19; Subfamily R:TCRβ V27; Subfamily S:TCRβ V28; Subfamily T: TCRβ V24; Subfamily U: TCRβ V20; Subfamily V: TCRβ V25; and Subfamily W:TCRβ V29 subfamily. Subfamily members are described in detail herein in the Section titled “TCR beta V (TCRβV)”.

FIGS. 2A-2C show human CD3+ T cells activated by anti-TCR Vβ13.1 antibody (BHM1709) for 6-days. Human CD3+ T cells were isolated using magnetic-bead separation (negative selection) and activated with immobilized (plate-coated) anti-TCR Vβ13.1 (BHM1709) or anti-CD3ε (OKT3) antibodies at 100 nM for 6 days. FIG. 2A shows two scatter plots (left: activated with OKT3; and right: activated with BHM1709) of expanded T cells assessed for TCR V013.1 surface expression using anti-TCR V013.1 (BHM1709) followed by a secondary fluorochrome-conjugated antibody for flow cytometry analysis. FIG. 2B shows percentage (%) of TCR Vβ13.1 positive T cells activated by anti-TCR Vβ13.1 (BHM1709) or anti-CD3e (OKT3) plotted against total T cells (CD3+). FIG. 2C shows relative cell count acquired by counting the number of events in each T cell subset gate (CD3 or TCR Vβ13.1) for 20 seconds at a constant rate of 60 μl/min. Data shown as mean value from 3 donors.

FIGS. 3A-3B show cytolytic activity of human CD3+ T cells activated by anti-TCR Vβ13.1 antibody (BHM1709) against transformed cell line RPMI 8226. FIG. 3A depicts target cell lysis of human CD3+ T cells activated with BHM 1709 or OKT3. Human CD3+ T cells were isolated using magnetic-bead separation (negative selection) and activated with immobilized (plate-coated) BHM1709 or OKT3 at the indicated concentrations for 4 days prior to co-culture with RPMI 8226 cells at a (E:T) ratio of 5:1 for 2 days. Samples were next analyzed for cell lysis of RPMI 8226 cells by FACS staining for CFSE/CD138-labeled, and membrane-impermeable DNA dyes (DRAQ7) using flow cytometry analysis. FIG. 3B shows target cell lysis of human CD3+ T cells activated with BHM 1709 or OKT3 incubated with RPMI-8226 at a (E:T) ratio of 5:1 for 6 days followed by cell lysis analysis of RPMI 8226 cells as described above. Percentage (%) target cell lysis was determined by normalizing to basal target cell lysis (i.e. without antibody treatment) using the following formula, ((x-basal)/(100%-basal), where x is cell lysis of sample). Data shown is a representative of n=1 donor.

FIGS. 4A-4B show IFNγ production by human PBMCs activated with the indicated antibodies. Human PBMCs were isolated from whole blood from the indicated number of donors, followed by solid-phase (plate-coated) stimulation with the indicated antibodies at 100 Nm. Supernatant was collected on Days 1, 2, 3, 5, or 6. FIG. 4A is a graph comparing the production of IFNγ in human PBMCs activated with the antibodies indicated activated with anti-TCR Vβ13.1 antibodies (BHM1709 or BHM1710) or anti-CD3e antibodies (OKT3 or SP34-2) on Day 1, 2, 3, 5, or 6 post-activation. FIG. 4B shows IFNγ production in human PBMCs activated with the antibodies indicated activated with the indicated anti-TCR Vβ13.1 antibodies or anti-CD3e antibody (OKT3) on Day 1, 2, 3, 5, or 6 post-activation.

FIG. 5A shows IL-2 production by human PBMCs activated with the indicated antibodies. A similar experimental setup as described for FIGS. 4A-4B was used. FIG. 5B shows IL-2 production by human PBMCs activated with the indicated antibodies. A similar experimental setup as described for FIGS. 4A-4B was used.

FIG. 6A shows IL-6 production by human PBMCs activated with the indicated antibodies. A similar experimental setup as described for FIGS. 4A-4B was used. FIG. 6B shows IL-6 production by human PBMCs activated with the indicated antibodies. A similar experimental setup as described for FIGS. 4A-4B was used.

FIG. 7A shows TNF-alpha production by human PBMCs activated with the indicated antibodies. A similar experimental setup as described for FIGS. 4A-4B was used. FIG. 7B shows TNF-alpha production by human PBMCs activated with the indicated antibodies. A similar experimental setup as described for FIGS. 4A-4B was used.

FIG. 8A is a line graph showing IL-1beta production by human PBMCs activated with the indicated antibodies. FIG. 8B is a line graph showing IL-1beta production by human PBMCs activated with the indicated antibodies. A similar experimental setup as described for FIGS. 4A-4B was used.

FIG. 9A is a graph showing delayed kinetics of IFNγ secretion in human PMBCs from 4 donors activated by anti-TCR Vβ13.1 antibody BHM1709 when compared to PBMCs activated by anti-CD3e antibody OKT3. FIG. 9B is a graph showing delayed kinetics of IFNγ secretion in human PMBCs from 4 donors activated by anti-TCR Vβ13.1 antibody BHM1709 when compared to PBMCs activated by anti-CD3e antibody OKT3. Data shown is representative of n=8 donors.

FIG. 10 depicts increased CD8+ TSCM and TEMRA T cell subsets in human PBMCs activated by anti-TCR Vβ13.1 antibodies (BHM1709 or BHM1710) compared to PBMCs activated by anti-CD3e antibodies (OKT3 or SP34-2).

FIG. 11A depicts an exemplary T cell stimulation method. FIG. 11B depicts a graph of IFNγ production by human PBMCs activated with the indicated antibodies. FIG. 11C depicts a graph of IFNγ production by human PBMCs activated with the indicated antibodies using the method shown in FIG. 11A.

FIG. 12A depicts an exemplary T cell stimulation method. FIG. 12B depicts a graph of IL-6 production by human PBMCs activated with the indicated antibodies using the method shown in FIG. 12A. FIG. 12C depicts a graph of IL-1β production by human PBMCs activated with the indicated antibodies using the method shown in FIG. 12A. FIG. 12D depicts a graph of IFNγ production by human PBMCs activated with the indicated antibodies using the method shown in FIG. 12A.

FIG. 13 depicts an exemplary T cell receptor (TCR) described herein. The TCR comprises a TCRα polypeptide chain comprising an immunoglobulin variable heavy chain or immunoglobulin variable light chain, a TCRα constant domain, a TCRα transmembrane domain, a TCRα intracellular domain, and optionally one or more (e.g., 2 or 3) costimulatory domains; and a TCRβ polypeptide chain comprising an immunoglobulin variable heavy chain or immunoglobulin variable light chain, a TCRβ constant domain (TCRβ constant 1 domain or TCRβ constant 2 domain), a TCRα transmembrane domain, a TCRβ intracellular domain, and optionally one or more (e.g., 2 or 3) costimulatory domains, wherein the immunoglobulin domains forma an antigen binding domain.

FIG. 14 depicts an exemplary chimeric antigen receptor (CAR) described herein. The CAR comprises an antigen binding domain (e.g., a scFv), a TCRβ constant domain (TCRβ constant 1 domain or TCRβ constant 2 domain), a TCRα transmembrane domain, a TCRβ intracellular domain, and optionally one or more (e.g., 2 or 3) costimulatory domains.

FIG. 15 depicts an anti-CD19 chimeric antigen receptor (CAR) cassette used in Example 3. The CAR comprises an EF1A promoter, a CD8a signal peptide, FMC63 single chain Fv that binds CD19, a FLAG tag, a CD28 intracellular costimulatory domain, and a CD3ζ intracellular signaling domain.

FIG. 16 is a bar graph showing the number of live cells 6 days post activation of T cell or CAR T cell cultures from 1 of 3 donors (donor 010, donor 541, donor 871). One of three activation conditions was used. Condition 1: activation using equal amounts of TCRβV clonotype specific antibodies H131 and 16G8 (50 nM each in PBS); Condition 2: activation using equal amounts of TCRβV clonotype specific antibodies H131 and 16G8 (50 nM each in PBS) and IL2 (culture medium containing 300 U/mL rIL-2 (cat. #Pr21269, ProMab); Condition 3: activation using equal amounts of anti-CD3ε and anti-CD28 antibodies (50 nM each in PBS). The number of live cells was determined by FACS analysis.

FIG. 17 is a bar graph showing the number of live cells 9 days post activation of T cell or CAR T cell cultures from 1 of 3 donors (donor 010, donor 541, donor 871). One of three activation conditions was used. Condition 1: activation using equal amounts of TCRβV clonotype specific antibodies H131 and 16G8 (50 nM each in PBS); Condition 2: activation using equal amounts of TCRβV clonotype specific antibodies H131 and 16G8 (50 nM each in PBS) and IL2 (culture medium containing 300 U/mL rIL-2 (cat. #Pr21269, ProMab); Condition 3: activation using equal amounts of anti-CD3ε and anti-CD28 antibodies (50 nM each in PBS). The number of live cells was determined by FACS analysis.

FIG. 18 is a bar graph showing the number of CD3+ cells 9 days post activation of T cell or CAR T cell cultures from 1 of 3 donors (donor 010, donor 541, donor 871). One of three activation conditions was used. Condition 1: activation using equal amounts of TCRβV clonotype specific antibodies H131 and 16G8 (50 nM each in PBS); Condition 2: activation using equal amounts of TCRβV clonotype specific antibodies H131 and 16G8 (50 nM each in PBS) and IL2 (culture medium containing 300 U/mL rIL-2 (cat. #Pr21269, ProMab); Condition 3: activation using equal amounts of anti-CD3ε and anti-CD28 antibodies (50 nM each in PBS). The number of CD3+ cells was determined by FACS analysis.

FIG. 19 is a bar graph showing the ratio of CD4+ to CD8+ T cells 9 days post activation of T cell or CART cell cultures from 1 of 3 donors (donor 010, donor 541, donor 871). One of three activation conditions was used. Condition 1: activation using equal amounts of TCRβV clonotype specific antibodies H131 and 16G8 (50 nM each in PBS); Condition 2: activation using equal amounts of TCRβV clonotype specific antibodies H131 and 16G8 (50 nM each in PBS) and IL2 (culture medium containing 300 U/mL rIL-2 (cat. #Pr21269, ProMab); Condition 3: activation using equal amounts of anti-CD3ε and anti-CD28 antibodies (50 nM each in PBS). The ratio of CD4+ to CD8+ T cells was determined by FACS analysis.

FIG. 20 is a bar graph showing the percentage of TCRβV+ cells 9 days post activation of T cell or CAR T cell cultures from 1 of 3 donors (donor 010, donor 541, donor 871). One of three activation conditions was used. Condition 1: activation using equal amounts of TCRβV clonotype specific antibodies H131 and 16G8 (50 nM each in PBS); Condition 2: activation using equal amounts of TCRβV clonotype specific antibodies H131 and 16G8 (50 nM each in PBS) and IL2 (culture medium containing 300 U/mL rIL-2 (cat. #Pr21269, ProMab); Condition 3: activation using equal amounts of anti-CD3ε and anti-CD28 antibodies (50 nM each in PBS). The ratio of CD4+ to CD8+ T cells was determined by FACS analysis using a 16G8-PE labeled antibody.

FIG. 21 is a bar graph showing the percentage of CAR+ T cells 9 days post activation of T cells from 1 of 3 donors (donor 010, donor 541, donor 871). One of three activation conditions was used. Condition 1: activation using equal amounts of TCRβV clonotype specific antibodies H131 and 16G8 (50 nM each in PBS); Condition 2: activation using equal amounts of TCRβV clonotype specific antibodies H131 and 16G8 (50 nM each in PBS) and IL2 (culture medium containing 300 U/mL rIL-2 (cat. #Pr21269, ProMab); Condition 3: activation using equal amounts of anti-CD3ε and anti-CD28 antibodies (50 nM each in PBS). The ratio of CD4+ to CD8+ T cells was determined by FACS analysis using FLAG staining as the CAR construct as shown in FIG. 15 contains a FLAG tag.

FIG. 22 is a graphic depiction of T cell (e.g., CART cells) expansion protocols described herein. T cells expanded using clonotypic anti-TCRβV antibodies target and expand only a specific subset of T cells. In contrast to the use of anti-CD3ε antibodies that activate all T cells. Activation and expansion of T cells using the anti-TCRβV antibodies prevents systemic release of cytokines that can lead to toxicity (e.g., CRS) when administered to a subject.

FIG. 23 is a FACS plot showing the expansion of TCRvb 6-5+ T cells over 8 days using anti-TCRvb 6-5 v1.

FIG. 24 is a bar graph showing the expansion of TCRvb 6-5+ CD4+ T cells and TCRvb 6-5+ CD8+ T cells over 8 days using the anti-CD3ε antibody OKT3 (100 nM).

FIG. 25 is a bar graph showing the expansion of TCRvb 6-5+ CD4+ T cells and TCRvb 6-5+ CD8+ T cells over 8 days using the anti-TCRvb 6-5 v1 antibody (100 nM).

FIG. 26 is a FACS plot showing the showing the expansion of TCRvb 6-5+ T cells over 8 days using anti-TCRvb 6-5 v1 or the anti-CD3ε antibody OKT3.

FIG. 27A is a bar graph showing the percentage of TCRβV 6-5+ T cells in PBMC cultures after 8 days of culture with the indicated antibody. Data for 5 replicates are shown. FIG. 27B is a bar graph showing the percentage of TCRβV 6-5+ T cells in purified T cell cultures after 8 days of culture with the indicated antibody. Data for 5 replicates are shown.

FIG. 28A is a bar graph showing the relative count of TCRβV 6-5+ T cells in PBMC culture after 8 days of culture with the indicated antibody. FIG. 28B is a bar graph showing the relative count of TCRβV 6-5+ T cells in PBMC culture after 8 days of culture with the indicated antibody.

FIG. 29A is a bar graph showing the relative count of TCRβV 6-5+ T cells in a purified T cell culture after 8 days of culture with the indicated antibody. FIG. 29B is a bar graph showing the relative count of TCRβV 6-5+ T cells in a purified T cell culture after 8 days of culture with the indicated antibody.

FIG. 30 is a line graph showing the total CD3+ T cell count (fold increase) after 8 days of T cell culture with either the anti-CD3ε antibody OKT3 or the anti-TCRvb 6-5 v1 antibody.

FIG. 31 is a series of line graphs showing the kinetics of target cells by TCRβV 6-5 v1 activated T cells or anti-CD3ε (OKT3) activated T cells. T cells from three different donors were utilized (donor 6769, donor 9880, donor 5411).

FIG. 32A is a scatter plot showing the percent of target cell lysis by T cells by TCRβV 6-5 v1 activated T cells or anti-CD3ε (OKT3) activated T cells without T cell pre activation. The data is presented at day 6 of co-culture between target cells and effector T cells. FIG. 32B is a scatter plot showing the percent of target cell lysis by T cells by TCRβV 6-5 v1 activated T cells or anti-CD3ε (OKT3) activated T cells with 4 days of T cell pre activation. The data is presented at day 2 of co-culture between target cells and effector T cells (after 4 days of T cell pre-activation).

FIG. 33 is a scatter plot showing the percent of target cell lysis by T cells by TCRβV 6-5 v1 activated T cells or anti-CD3ε (OKT3) activated T cells with 4 days of T cell pre activation. The data is presented at day 2 of co-culture between target cells and effector T cells (after 4 days of T cell pre-activation).

FIG. 34 is a bar graph showing target cell lysis by T cells by TCRβV 6-5 v1 activated T cells or anti-CD3ε (OKT3) activated T cells (100 nM each antibody). The data includes seven replicates of each experimental condition.

FIG. 35 is a series of FACS plots that show the cell surface expression of CD3ε on CD4+ TCRβV 6-5⁻ or CD4+ TCRβV 6-5⁺ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 36 is a series of FACS plots that show the cell surface expression of CD3ε on CD8+ TCRβV 6-5⁻ or CD8+ TCRβV 6-5⁺ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 37 is a series of FACS plots that show the cell surface expression of TCRβV on CD4+ TCRβV 6-5⁻ or CD4+ TCRβV 6-5⁻ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 38 is a series of FACS plots that show the cell surface expression of TCRβV on CD8+ TCRβV 6-5⁻ or CD8+ TCRβV 6-5⁻ T cells activated with either SP34-2 (anti-CD3ε antibody) or anti-TCRβV 6-5 v1 (anti-TCRβV 6-5 antibody) at days 0, 1, 2, 4, 6, or 8 post antibody activation.

FIG. 39A shows FACS plot of TCRβV 6-5⁺ cynomolgus T cell expansion either unstimulated (left) or stimulated with anti-TCRβV 6-5 v1 (right) 7 days post activation of cynomolgus PBMCs. PBMCs from Donor DW8N (fresh PBMC sample, male, age 8, weight 7.9 kgs) were used. FIG. 39B shows FACS plot of TCRβV 6-5⁺ cynomolgus T cell expansion either unstimulated (left) or stimulated with anti-TCRβV 6-5 v1 (right) 7 days post activation of cynomolgus PBMCs. PBMCs from Donor G709 (cryopreserved sample, male, age 6, weight 4.7 kgs) were used.

FIG. 40 shows FACS plot and corresponding microscopy images of TCRβV 6-5⁺ cynomolgus T cell expansion either unstimulated (left), stimulated with SP34-2 (anti-CD3ε antibody) (middle); or stimulated with anti-TCRβV 6-5 v1 (right) post activation of cryopreserved donor DW8N cynomolgus PBMCs. The microscopy images show the cell cluster formation (indicated by circles).

FIG. 41 shows a schematic of FACS plot showing the FACS gating/staining of PBMCs prior γ6 T cell purification.

FIG. 42 shows a schematic of FACS plot showing the FACS gating/staining of purified γ6 T cell population.

FIG. 43 show activation of purified γ6 T cell population with anti-CD3ε antibody (SP34-2) (left) or anti-TCRβV antibody (anti-TCRβV 6-5 v1) (right).

FIG. 44A shows the release of IFNγ from purified γ6 T cell populations activated with anti-CD3ε antibody (SP34-2), anti-TCRβV antibody (anti-TCRβV 6-5 v1), or unstimulated. FIG. 44B shows the release of TNFα from purified γ6 T cell populations activated with anti-CD3ε antibody (SP34-2), anti-TCRβV antibody (anti-TCRβV 6-5 v1), or unstimulated. FIG. 44C shows the release of IL-2 from purified γ6 T cell populations activated with anti-CD3ε antibody (SP34-2), anti-TCRβV antibody (anti-TCRβV 6-5 v1), or unstimulated. FIG. 44D shows the release of IL-17A from purified γ6 T cell populations activated with anti-CD3ε antibody (SP34-2), anti-TCRβV antibody (anti-TCRβV 6-5 v1), or unstimulated. FIG. 44E shows the release of IL-1α from purified γ6 T cell populations activated with anti-CD3ε antibody (SP34-2), anti-TCRβV antibody (anti-TCRβV 6-5 v1), or unstimulated. FIG. 44F shows the release of IL-1β from purified γ6 T cell populations activated with anti-CD3ε antibody (SP34-2), anti-TCRβV antibody (anti-TCRβV 6-5 v1), or unstimulated. FIG. 44G shows the release of IL-6 from purified γ6 T cell populations activated with anti-CD3ε antibody (SP34-2), anti-TCRβV antibody (anti-TCRβV 6-5 v1), or unstimulated. FIG. 44H shows the release of IL-1β from purified γ6 T cell populations activated with anti-CD3ε antibody (SP34-2), anti-TCRβV antibody (anti-TCRβV 6-5 v1), or unstimulated.

FIG. 45 shows the relative representations of all TCR alpha V segments (TRAV group of genes) and their variants (top), all TCR beta V segment 6-5 variants (TRBV6-5 gene) (bottom left), and all TCR beta V segments and variants excluding 6-5 (bottom right).

FIG. 46A is a FACS plot showing phenotypic markers of CD4+ T cells expanded with anti-TCRβV antibody (anti-TCRβV 6-5 v1). Defined phenotypes include TEMRA (top left), Naïve/TSCM (top right), TEM (bottom left), and TCM (bottom right). FIG. 46B is a FACS plot showing phenotypic markers of CD4+ T cells expanded with anti-CD3ε antibody (OKT3). Defined phenotypes include TEMRA (top left), Naïve/TSCM (top right), TEM (bottom left), and TCM (bottom right).

FIG. 47A is a FACS plot showing phenotypic markers of CD8+ T cells expanded with anti-TCRβV antibody (anti-TCRβV 6-5 v1). Defined phenotypes include TEMRA (top left), Naïve/TSCM (top right), TEM (bottom left), and TCM (bottom right). FIG. 47B is a FACS plot showing phenotypic markers of CD8+ T cells expanded with anti-CD3ε antibody (OKT3). Defined phenotypes include TEMRA (top left), Naïve/TSCM (top right), TEM (bottom left), and TCM (bottom right).

FIG. 48A is a bar graph showing the percentage of PD1 expressing CD4+ T cells from T cell cultures activated with anti-TCRβV antibody (anti-TCRβV 6-5 v1), anti-CD3ε antibody (OKT3), or unstimulated. FIG. 48B is a bar graph showing the percentage of PD1 expressing CD8+ T cells from T cell cultures activated with anti-TCRβV antibody (anti-TCRβV 6-5 v1), anti-CD3ε antibody (OKT3), or unstimulated.

FIG. 49A is a bar graph showing the expression of Ki-67 by CD4+ T cells from T cell cultures activated with anti-TCRβV antibody (anti-TCRβV 6-5 v1), anti-CD3ε antibody (OKT3), or unstimulated. FIG. 49B is a bar graph showing the expression of Ki-67 by CD8+ T cells from T cell cultures activated with anti-TCRβV antibody (anti-TCRβV 6-5 v1), anti-CD3ε antibody (OKT3), or unstimulated.

FIG. 50A is a FACS plot showing the percentage of TEMRA-like CD8+ T cells activated using anti-TCRβV antibody (anti-TCRβV 6-5 v1) that express CD57 (18.7%). FIG. 50B is a FACS plot showing the percentage of TEM-like CD8+ T cells activated using anti-CD3ε antibody (OKT3) that express CD57 (46.8%) and the percentage of TCM-like CD8+ T cells activated using anti-CD3ε antibody (OKT3) that express CD57 (18.9%).

FIG. 51 shows a series of FACS plots showing the expression of expression of CD27 and by CD4+ (top) or CD8+ (bottom) T cells from T cell cultures activated with anti-TCRβV antibody (anti-TCRβV 6-5 v1), anti-CD3ε antibody (OKT3), or unstimulated.

FIG. 52 shows a series of FACS plots showing the expression of expression of OX40, 41BB, and ICOS by CD4+ (top) or CD8+ (bottom) T cells from T cell cultures activated with anti-TCRβV antibody (anti-TCRβV 6-5 v1), anti-CD3ε antibody (OKT3), or unstimulated.

FIG. 53 shows a series of FACS plots showing the expression level of TCRβV6-5 by Jurkat cells passaged through 11 (P11), 15 (P15), and 21 (P21) passages.

FIG. 54 shows a series of FACS plots showing the percentage of CD3+ (CD4 gated) TCRβV 6-5+ T cells 1, 2, 3, 4, 5, 6, and 8 days port activation with BCMA and the anti-TCR Vβ antibody anti-TCR Vβ 6-5 v1.

FIG. 55A shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 0 post activation. FIG. 55B shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 1 post activation. FIG. 55C shows a series of FACS plots showing the percentage of CD4+ T cells expanded using isotype control (IgG1 N297A), anti-TCRβV (anti-TCR 6-5 v1), or anti-CD3ε (OKT3) antibodies on day 2 post activation. FIG. 55D shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 3 post activation. FIG. 55E shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 4 post activation. FIG. 55F shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 5 post activation. FIG. 55G shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 6 post activation. FIG. 55H shows a series of FACS plots showing the percentage of CD4+ T cells expanded using isotype control (IgG1 N297A), anti-TCRβV (anti-TCR Vβ 6-5 v1), or anti-CD3ε (OKT3) antibodies on day 8 post activation.

FIG. 56A is a map showing differential gene expression between cells activated with anti-TCRvb 6-5 v1 antibody versus unstimulated. FIG. 56B is a map showing differential gene expression between cells activated with cells activated with OKT3 versus unstimulated. FIG. 56C is a map showing differential gene expression between cells activated with cells activated with SP34-2 versus unstimulated. FIG. 56D is a map showing differential gene expression between cells activated with and cells activated with anti-TCRvb 6-5 v1 antibody versus OKT3 FIG. 56E is a map showing no differential gene expression detected between cells activated with OKT3 versus SP34-2.

FIG. 57A shows the distribution of genes differentially upregulated post T cell stimulation with the indicated antibody. FIG. 57B shows the distribution of genes differentially downregulated post T cell stimulation with the indicated antibody. FIG. 57C shows the distribution of genes differentially upregulated or downregulated post T cell stimulation with the indicated antibody. FIG. 57D shows the distribution of genes differentially upregulated or downregulated post T cell stimulation with the indicated antibody.

FIG. 58 shows a heat map of pathway scores for genes differentially regulated and related to various cellular pathways. The purified T cell samples include unstimulated (n=3), OKT3 stimulated (n=3), SP34-2 stimulated (n=3), and anti-TCRβV 6-5 v1 stimulated (n=3).

FIG. 59A shows a plot of cytokines and chemokine pathways upregulated or downregulated by activation with the indicated antibodies or unstimulated. FIG. 59B shows a plot of TNF superfamily and interleukin pathways upregulated or downregulated by activation with the indicated antibodies or unstimulated. FIG. 59C shows a plot of T cell function and senescence pathways upregulated or downregulated by activation with the indicated antibodies or unstimulated. FIG. 59D shows a plot of cell cycle and cytotoxicity pathways upregulated or downregulated by activation with the indicated antibodies or unstimulated.

FIG. 60A shows a plot of T cell function pathway upregulated or downregulated by activation with the indicated antibodies or unstimulated. FIG. 60B shows a plot of senescence pathway upregulated or downregulated by activation with the indicated antibodies or unstimulated.

FIG. 61A shows the differential regulation of granzyme B in cells activated with the indicated antibody or unstimulated. P≤0.01****; p≤0.05***; p≤0.5*; p≤0.5 ns. FIG. 61B shows the differential regulation of perforin in cells activated with the indicated antibody or unstimulated. P≤0.01****; p≤0.05***; p≤0.5*; p≤0.5 ns. FIG. 61C shows the differential regulation of IL-2 in cells activated with the indicated antibody or unstimulated. P≤0.01****; p≤0.05***; p≤0.5*; p≤0.5^{ns}. FIG. 61D shows the differential regulation of LIF in cells activated with the indicated antibody or unstimulated. P≤0.01****; p≤0.05***; p≤0.5*; p≤0.5 ns. FIG. 61E shows the differential regulation of IFNγ in cells activated with the indicated antibody or unstimulated. P≤0.01****; p≤0.05***; p≤0.5*; p≤0.5 ns. FIG. 61F shows the differential regulation of IL-22 in cells activated with the indicated antibody or unstimulated. P≤0.01****; p≤0.05***; p≤0.5*; p≤0.5 ns. FIG. 61G shows the differential regulation of CD40LG in cells activated with the indicated antibody or unstimulated. P≤0.01****; p≤0.05***; p≤0.5*; p≤0.5 ns. FIG. 61H shows the differential regulation of ICOS in cells activated with the indicated antibody or unstimulated. P≤0.01****; p≤0.05***; p≤0.5*; p≤0.5^{ns}. FIG. 61I shows the differential regulation of CXCL9 in cells activated with the indicated antibody or unstimulated. P≤0.01****; p≤0.05***; p≤0.5*; p≤0.5 ns. FIG. 61J shows the differential regulation of CXCL10 in cells activated with the indicated antibody or unstimulated. P≤0.01****; p≤0.05***; p≤0.5*; p≤0.5^{ns}.

FIG. 62 shows a graph from a principal component analysis (PCA) of genes related to T cell activation and exhaustion differentially expressed after activation of T cells with the indicated antibody.

FIG. 63 shows a graph from a principal component analysis (PCA) of genes related to co-stimulatory expressed after activation of T cells with the indicated antibody.

FIG. 64 shows a graph from a principal component analysis (PCA) of genes related to regulatory functions expressed after activation of T cells with the indicated antibody.

FIG. 65A is a bar graph showing ATP production from glycolysis of T cell cultures activated with the indicated antibodies. FIG. 65B is a bar graph showing ATP production from oxidative phosphorylation of T cell cultures activated with the indicated antibodies.

FIG. 66 is a line graph showing the oxygen consumption rate (OCR) of T cells from about 0 to 75 minutes activated with the indicated antibody.

FIG. 67A shows the oxygen consumption rate (OCR) of T cells activated with the indicated antibody during basal respiration. FIG. 67B shows the oxygen consumption rate (OCR) of T cells activated with the indicated antibody during maximal respiration. FIG. 67C shows the oxygen consumption rate (OCR) of T cells activated with the indicated antibody during spare respiratory capacity. FIG. 67D is a line graph indicates the areas of basal respiration and maximal respiration as shown in FIG. 67A and FIG. 67B, respectively.

FIG. 68A is a bar graph showing ATP production from glycolysis of T cell cultures activated with anti-TCRβV 6-5 v1 and re-stimulated with the indicated antibody. FIG. 68B is a bar graph showing ATP production from oxidative phosphorylation of T cell cultures activated with anti-TCRβV 6-5 v1 and re-stimulated with the indicated antibody.

FIG. 69A is a FACS plot showing the percentage of CMV (pp65) specific anti-TCRβV 6-5 v1 activated TCRvβ 6-5+ CD8+ T cells from the indicated donor (donor 14497 or donor 14693). FIG. 69B is a FACS plot showing the percentage of EBV (LMP2) specific anti-TCRβV 6-5 v1 activated TCRvβ 6-5+ CD8+ T cells from the indicated donor (donor 14497 or donor 14693). FIG. 69C is a FACS plot showing the percentage of EBV (mixed peptide) specific anti-TCRβV 6-5 v1 activated TCRvβ 6-5+ CD8+ T cells from the indicated donor (donor 14497 or donor 14693). FIG. 69D is a FACS plot showing the percentage of influenza specific anti-TCRβV 6-5 v1 activated TCRvβ 6-5+ CD8+ T cells from the indicated donor (donor 14497 or donor 14693). FIG. 69E is a FACS plot showing the percentage of influenza specific anti-TCRβV 6-5 v1 activated TCRvβ 6-5+ CD8+ T cells from the indicated donor (donor 11011). FIG. 69F is a bar graph showing the percent viral peptide specific (CD8+ T cells) for in the indicated virus.

FIG. 70 is a FACS plot showing the percentage of NK cells expanded from T cell cultures activated with the indicated antibody.

FIG. 71 is a bar graph showing the number of NK cells expanded from T cell cultures activated with the indicated antibody.

FIG. 72 shows a series of FACS plots showing NK cell proliferation induced by T cell cultures activated with the indicated antibody.

FIG. 73 is a schematic showing an assay described in Example for determining NK cell mediated lysis of target K562 cells.

FIG. 74 is a bar graph showing the percent target cell lysis mediated by NK cells activated by PBMCs activated with the indicated antibody.

FIG. 75 is a bar graph showing the level of secreted IFNγ by T cells activated/expanded with the indicated antibody (anti-TCRβV 6-5 v1, OKT3, or SP34) and cultured with said antibody for the indicated number of days (1, 3, or 5).

FIG. 76 is a bar graph showing the level of secreted IL-2 by T cells activated/expanded with the indicated antibody (anti-TCRβV 6-5 v1, OKT3, or SP34) and cultured with said antibody for the indicated number of days (1, 3, or 5).

FIG. 77 is a bar graph showing the level of secreted IL-15 by T cells activated/expanded with the indicated antibody (anti-TCRβV 6-5 v1, OKT3, or SP34) and cultured with said antibody for the indicated number of days (1, 3, or 5).

FIG. 78 is a bar graph showing the level of secreted IL-1β by T cells activated/expanded with the indicated antibody (anti-TCRβV 6-5 v1, OKT3, or SP34) and cultured with said antibody for the indicated number of days (1, 3, or 5).

FIG. 79 is a bar graph showing the level of secreted IL-6 by T cells activated/expanded with the indicated antibody (anti-TCRβV 6-5 v1, OKT3, or SP34) and cultured with said antibody for the indicated number of days (1, 3, or 5).

FIG. 80 is a bar graph showing the level of secreted IL-1β by T cells activated/expanded with the indicated antibody (anti-TCRβV 6-5 v1, OKT3, or SP34) and cultured with said antibody for the indicated number of days (1, 3, or 5).

FIG. 81 is a bar graph showing the level of the indicated cytokine secreted by T cells activated/expanded with the indicated antibody (anti-TCRβV 6-5 v1 or SP34). The data includes use of 17 individual PBMC donors.

FIG. 82A is a bar graph showing the level of secreted IFNγ by T cells activated/expanded with the indicated antibody (anti-TCRβV 6-5 v1 or OKT3) and cultured with said antibody for the indicated number of days (1, 2, 3, 5, or 6). FIG. 82B is a bar graph showing the level of secreted IL-1β by T cells activated/expanded with the indicated antibody (anti-TCRβV 6-5 v1 or OKT3) and cultured with said antibody for the indicated number of days (1, 2, 3, 5, or 6). FIG. 82C is a bar graph showing the level of secreted IL-4 by T cells activated/expanded with the indicated antibody (anti-TCRβV 6-5 v1 or OKT3) and cultured with said antibody for the indicated number of days (1, 2, 3, 5, or 6). FIG. 82D is a bar graph showing the level of secreted IL-6 by T cells activated/expanded with the indicated antibody (anti-TCRβV 6-5 v1 or OKT3) and cultured with said antibody for the indicated number of days (1, 2, 3, 5, or 6). FIG. 82E is a bar graph showing the level of secreted IL-1β by T cells activated/expanded with the indicated antibody (anti-TCRβV 6-5 v1 or OKT3) and cultured with said antibody for the indicated number of days (1, 2, 3, 5, or 6). FIG. 82F is a bar graph showing the level of secreted TNFα by T cells activated/expanded with the indicated antibody (anti-TCRβV 6-5 v1 or OKT3) and cultured with said antibody for the indicated number of days (1, 2, 3, 5, or 6). FIG. 82G is a bar graph showing the level of secreted IL-2 by T cells activated/expanded with the indicated antibody (anti-TCRβV 6-5 v1 or OKT3) and cultured with said antibody for the indicated number of days (1, 2, 3, 5, or 6).

FIG. 83A is a bar graph showing the level of secreted IFNγ by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 83B is a bar graph showing the level of secreted IL-1β by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 83C is a bar graph showing the level of secreted IL-4 by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 83D is a bar graph showing the level of secreted IL-6 by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6).

FIG. 83E is a bar graph showing the level of secreted IL-1β by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 83F is a bar graph showing the level of secreted TNFα by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6). FIG. 83G is a bar graph showing the level of secreted IL-2 by T cells activated/expanded with the indicated antibody (anti-TCRβV 6-5 v1, anti-TCRβV 6-5 v1, OKT3, SP34-2, or isotype control) and cultured with said antibody for the indicated number of days (1, 2, 3, 5, or 6).

FIG. 84A is a bar graph showing the level of secreted IFNγ by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 84B is a bar graph showing the level of secreted IL-1β by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 84C is a bar graph showing the level of secreted IL-4 by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 84D is a bar graph showing the level of secreted IL-6 by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 84E is a bar graph showing the level of secreted IL-1β by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 84F is a bar graph showing the level of secreted TNFα by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8). FIG. 84G is a bar graph showing the level of secreted IL-2 by T cells activated/expanded with the indicated antibody (anti-TCRβV 6-5 v1, anti-TCRβV 6-5 v1, OKT3, or SP34-2) and cultured with said antibody for the indicated number of days (1, 2, 3, 4, 5, 6, or 8).

FIG. 85A is a bar graph showing the level of secreted IL-17A by T cells activated/expanded with the indicated antibody (anti-TCRβV 6-5 v1, OKT3, or SP34-2) and cultured with said antibody for the indicated number of days (2, 5, or 7). FIG. 85B is a bar graph showing the level of secreted IL-17A by T cells activated/expanded with the indicated antibody (anti-TCRβV 6-5 v1, OKT3, or SP34-2) and cultured with said antibody for the indicated number of days (2, 5, or 8). FIG. 85C is a bar graph showing the level of secreted IL-17A by T cells activated/expanded with the indicated antibody (anti-TCRβV 6-5 v1, OKT3, or SP34-2) and cultured with said antibody for the indicated number of days (2, 5, or 7). FIG. 85D is a bar graph showing the level of secreted IL-17A by T cells activated/expanded with the indicated antibody (anti-TCRβV 6-5 v1 or SP34-2) and cultured with said antibody for the indicated number of days (1, 3, 5, or 7).

FIG. 86A is a bar graph showing the level of secreted IFNγ by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86B is a bar graph showing the level of secreted IL-1β by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86C is a bar graph showing the level of secreted IL-4 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86D is a bar graph showing the level of secreted IL-6 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86E is a bar graph showing the level of secreted IL-1β by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86F is a bar graph showing the level of secreted TNFα by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86G is a bar graph showing the level of secreted IL-2 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86H is a bar graph showing the level of secreted IL-12p70 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86I is a bar graph showing the level of secreted IL-13 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86J is a bar graph showing the level of secreted IL-8 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86K is a bar graph showing the level of secreted exotaxin by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86L is a bar graph showing the level of secreted exotoxin-3 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86M is a bar graph showing the level of secreted IL-8 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86N is a bar graph showing the level of secreted IP-10 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86O is a bar graph showing the level of secreted MCP-1 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86P is a bar graph showing the level of secreted MCP-4 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86Q is a bar graph showing the level of secreted MDC by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86R is a bar graph showing the level of secreted MIP-la by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86S is a bar graph showing the level of secreted MIP-1b by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86T is a bar graph showing the level of secreted TARC by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86U is a bar graph showing the level of secreted GMCSF by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86V is a bar graph showing the level of secreted IL-12-23p40 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86W is a bar graph showing the level of secreted IL-15 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86X is a bar graph showing the level of secreted IL-16 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86Y is a bar graph showing the level of secreted IL-17a by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86Z is a bar graph showing the level of secreted IL-1α by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86AA is a bar graph showing the level of secreted IL-5 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86BB is a bar graph showing the level of secreted IL-7 by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86CC is a bar graph showing the level of secreted TNF-β by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8). FIG. 86DD is a bar graph showing the level of secreted VEGF by T cells activated/expanded with the indicated antibody (isotype control; anti-TCRβV 6-5 v1 with anti-BCMA antibody; anti-TCRβV 6-5 v1; anti-TCRβV 123/4 v1, or SP34-2) and cultured with said antibody for the indicated number of days (1, 2, 3, 4, 5, 6, or 8).

FIG. 87A is a bar graph showing the level of secreted IFN-γ by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87B is a bar graph showing the level of secreted IFN-γ by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87C is a bar graph showing the level of secreted IL-1b by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87D is a bar graph showing the level of secreted IL-6 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87E is a bar graph showing the level of secreted IL-1β by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87F is a bar graph showing the level of secreted IL-15 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87G is a bar graph showing the level of secreted IL-17A by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87H is a bar graph showing the level of secreted IL-1α by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87I is a bar graph showing the level of secreted IL-1b by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87J is a bar graph showing the level of secreted IL-2 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87K is a bar graph showing the level of secreted IL-4 by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7). FIG. 87L is a bar graph showing the level of secreted TNF-α by T cells activated/expanded with the indicated antibody (anti-TCRβV 6-5 v1 (plate coated), anti-CD3ε (plate coated), anti-TCRβV 6-5 v1 (in solution), or anti-CD3ε (in solution) and cultured with said antibody for the indicated number of days (1, 3, 5, or 7).

FIG. 88 shows a graphical representation of the relation of sequences between different TCRVB clonotype subfamilies.

FIG. 89A is a bar graph showing the percentage of cytokine release from PBMCs activated/expanded for eight days using the indicated antibody (anti-TCRβV 12-3/4 v1 or SP34-2). FIG. 89B is a bar graph showing the percentage of cytokine release from PBMCs activated/expanded for eight days using the indicated antibody (anti-TCRβV 5 or SP34-2). FIG. 89C is a bar graph showing the percentage of cytokine release from PBMCs activated/expanded for eight days using the indicated antibody (anti-TCRβV 10 or SP34-2).

FIG. 90 shows a series of FACS plots showing the proliferation of NK cells from PBMC cultures activated/expanded with the indicated antibody (isotype control or OKT3). PBMCs from three donors (D1, D2, and D3) were analyzed.

FIG. 91 shows a series of FACS plots showing the proliferation of NK cells from PBMC cultures activated/expanded with the indicated antibody (anti-TCRvβ 12-3/4 v1 or anti-TCRvβ 12-3/4 v2). PBMCs from three donors (D1, D2, and D3) were analyzed.

FIG. 92 shows a series of FACS plots showing the proliferation of NK cells from PBMC cultures activated/expanded with the indicated antibody (anti-TCRvβ 12-3/4 v3 or SP34-2). PBMCs from three donors (D1, D2, and D3) were analyzed.

FIG. 93A a bar graph showing the level of secreted IFNγ by T cells activated/expanded with the indicated antibody for the indicated number of days (3 or 6). FIG. 93B a bar graph showing the level of secreted IL-1β by T cells activated/expanded with the indicated antibody for the indicated number of days (3 or 6). FIG. 93C a bar graph showing the level of secreted IL-17A by T cells activated/expanded with the indicated antibody for the indicated number of days (3 or 6). FIG. 93D a bar graph showing the level of secreted IL-1α by T cells activated/expanded with the indicated antibody for the indicated number of days (3 or 6). FIG. 93E a bar graph showing the level of secreted IL-1β by T cells activated/expanded with the indicated antibody for the indicated number of days (3 or 6). FIG. 93F a bar graph showing the level of secreted IL-6 by T cells activated/expanded with the indicated antibody for the indicated number of days (3 or 6). FIG. 93G a bar graph showing the level of secreted TNFα by T cells activated/expanded with the indicated antibody for the indicated number of days (3 or 6). FIG. 93H a bar graph showing the level of secreted IL-2 by T cells activated/expanded with the indicated antibody for the indicated number of days (3 or 6).

FIG. 94 is a bar graph summarizing data from FACS analysis of PBMCs activated/expanded for 6 days using the indicated anti-TCRVβ antibody.

FIG. 95A a bar graph showing the level of secreted IFNγ by T cells activated/expanded with the indicated antibody for the indicated number of days (1, 3, 5, or 7). FIG. 95B a bar graph showing the level of secreted IL-1β by T cells activated/expanded with the indicated antibody for the indicated number of days (1, 3, 5, or 7). FIG. 95C a bar graph showing the level of secreted IL-17A by T cells activated/expanded with the indicated antibody for the indicated number of days (1, 3, 5, or 7). FIG. 95D a bar graph showing the level of secreted IL-1α by T cells activated/expanded with the indicated antibody for the indicated number of days (1, 3, 5, or 7). FIG. 95E a bar graph showing the level of secreted IL-1β by T cells activated/expanded with the indicated antibody for the indicated number of days (1, 3, 5, or 7). FIG. 95F a bar graph showing the level of secreted IL-6 by T cells activated/expanded with the indicated antibody for the indicated number of days (1, 3, 5, or 7). FIG. 95G a bar graph showing the level of secreted IL-4 by T cells activated/expanded with the indicated antibody for the indicated number of days (1, 3, 5, or 7). FIG. 95H a bar graph showing the level of secreted IL-2 by T cells activated/expanded with the indicated antibody for the indicated number of days (1, 3, 5, or 7).

FIG. 96 is a bar graph summarizing data from FACS analysis of PBMCs activated/expanded for 7 days using the indicated anti-TCRVβ antibody.

FIG. 97A is a bar graph showing the level of secreted IFNγ by T cells activated/expanded with the indicated antibody for the indicated number of days (3 or 6). FIG. 97B a bar graph showing the level of secreted IL-1β by T cells activated/expanded with the indicated antibody for the indicated number of days (3 or 6). FIG. 97C a bar graph showing the level of secreted IL-17A by T cells activated/expanded with the indicated antibody for the indicated number of days (3 or 6). FIG. 97D a bar graph showing the level of secreted IL-1α by T cells activated/expanded with the indicated antibody for the indicated number of days (3 or 6). FIG. 97E a bar graph showing the level of secreted IL-1β by T cells activated/expanded with the indicated antibody for the indicated number of days (3 or 6). FIG. 97F a bar graph showing the level of secreted IL-6 by T cells activated/expanded with the indicated antibody for the indicated number of days (3 or 6). FIG. 97G a bar graph showing the level of secreted IL-4 by T cells activated/expanded with the indicated antibody for the indicated number of days (3 or 6). FIG. 97H a bar graph showing the level of secreted TNFα by T cells activated/expanded with the indicated antibody for the indicated number of days (3 or 6). FIG. 97I a bar graph showing the level of secreted IL-2 by T cells activated/expanded with the indicated antibody for the indicated number of days (3 or 6).

FIG. 98 is a FACS plot showing the showing the ability of MH3-2 to bind PBMCs from one of two donors when the PBMCs are either preincubated with TM23 or not (MH3-2 Alone).

FIG. 99 is a FACS plot showing the ability of MH3-2 to bind PBMCs from one of two donors when the PBMCs are either preincubated with TM23 or not (MH3-2 Alone).

FIG. 100A is a bar graph showing the polyfunctional strength index (PSI) of PBMC CD4+ T cells, CD4+ T cells expanded with anti-CD3 antibody, (CD3 Expanded T cells), and CD4+ T cells expanded with anti-TCRVβ 6-5 antibody (Drug Expanded T cells). The Effector mediators are Granzyme B, IFNγ, MIP-1a, perforin, TNFα, and TNFβ. The Stimulatory mediators are IL-5. The Chemoattractive mediators are MIP-lb. FIG. 100B is a bar graph showing the polyfunctional strength index (PSI) of PBMC CD8+ T cells, CD8+ T cells expanded with anti-CD3 antibody, (CD3 Expanded T cells), and CD8+ T cells expanded with anti-TCRVβ 6-5 antibody (Drug Expanded T cells). The Effector mediators are Granzyme B, IFNγ, MIP-1a, perforin, and TNFβ. The Chemoattractive mediators are MIP-1b and RANTES.

FIG. 101A is a line graph showing the number of cells at Day 0, Day 7, Day 9, and Day 11 of CAR T cells cultured with the indicated antibody and medium (or no virus control) produced from Donor 177 PBMCs. FIG. 101B is a line graph showing the number of cells at Day 0, Day 7, Day 9, and Day 11 of CAR T cells cultured with the indicated antibody and medium (or no virus control) produced from Donor 178 PBMCs. FIG. 101C is a line graph showing the number of cells at Day 0, Day 7, Day 9, and Day 11 of CAR T cells cultured with the indicated antibody and medium (or no virus control) produced from Donor 890 PBMCs.

FIG. 102 is a schematic of the flow cytometry protocol for staining CAR-T cells at Day 11.

FIG. 103 is a bar graph showing the CAR-T cell frequency at Day 11 of CAR T cells cultured with the indicated antibody and medium (or no virus control).

FIG. 104A is a bar graph showing the percentage of CAR-T cells of Teff, Tem, Tcm, and Tn phenotype based on CD45RO-APC and CD62L-FITC staining of CAR-T cells produced from PBMCs of Donor 177 at Day 11. FIG. 104B is a bar graph showing the percentage of CAR-T cells of Teff, Tem, Tcm, and Tn phenotype based on CD45RO-APC and CD62L-FITC staining of CAR-T cells produced from PBMCs of Donor 178 at Day 11. FIG. 104C is a bar graph showing the percentage of CAR-T cells of Teff, Tem, Tcm, and Tn phenotype based on CD45RO-APC and CD62L-FITC staining of CAR-T cells produced from PBMCs of Donor 890 at Day 11.

FIG. 105A shows the cytotoxicity of CAR-T cells made by activation with the indicated antibody and medium from PBMCs of Donor 177. FIG. 105B shows the cytotoxicity of CAR-T cells made by activation with the indicated antibody and medium from PBMCs of Donor 178. FIG. 105C shows the cytotoxicity of CAR-T cells made by activation with the indicated antibody and medium from PBMCs of Donor 890. FIG. 105D is a bar graph showing a summary of cytotoxicity of CAR-T cells made by activation with the indicated antibody and medium at 8 hours post addition of the target cells.

FIG. 105E is a bar graph showing a summary of cytotoxicity of CAR-T cells made by activation with the indicated antibody and medium at 24 hours post addition of the target cells.

FIG. 106 is a bar graph showing the production of IFNγ by CAR-T cells activated with the indicated antibody and used in cytotoxicity assay.

FIG. 107 shows a sequence alignment of 8 functional human TCRVβ6 family sequences—the boxes show three unique amino acids in subfamily 6-5 (SEQ ID NOS 379-386, respectively, in order of appearance).

FIG. 108A is a line graph showing H131 antibody binding to WT TCR receptor. FIG. 108B is a line graph showing H131 antibody binding to Q78A TCR receptor. FIG. 108C is a line graph showing H131 antibody binding to L101A TCR receptor. FIG. 108D is a line graph showing H131 antibody binding to S102A TCR receptor.

### DETAILED DESCRIPTION

Current methods of expanding T cells ex vivo comprise contacting the T cells with an antibody molecule that specifically binds the CD3ε subunit of the T cell receptor (TCR) alone or in combination with targeting the co-stimulatory receptor CD28. However, there are limitations to this approach which may prevent the full realization of the therapeutic potential for such T cell therapies. Previous studies have shown that even low “activating” doses of anti-CD3ε monoclonal antibodies (mAbs) can cause long-term T cell dysfunction and exert immunosuppressive effects. In addition, administration of T cells activated/expanded with anti-CD3ε antibodies have been associated with inflammatory side effects, including cytokine release syndrome (CRS), macrophage activation syndrome, neurological toxicities, and tumor lysis syndrome. The anti-CD3ε antibody activated T cells secrete proinflammatory cytokines, such as IFNγ, IL-1, IL-6 and TNF-α, or secrete proinflammatory cytokines (e.g., IFNγ) that activate antigen presenting cells, such as macrophages to secrete proinflammatory cytokines, such as IL-1, IL-6 and TNF-α, which induces cytokine release syndrome (CRS), macrophage activation syndrome, neurological toxicities, or tumor lysis syndrome. Thus, the need exists for developing antibodies that are capable of binding and activating only a subset of effector T cells, e.g., to reduce the CRS.

This disclosure is based, at least in part, on the unexpected discovery that T cells can be activated and expanded ex vivo using anti-TCRVβ antibodies; and that these T cells secrete substantially lower levels of proinflammatory cytokines associated with the induction of cytokine release syndrome (CRS), macrophage activation syndrome, neurological toxicities, and tumor lysis syndrome, such as IFNγ, IL-10, IL-17A, IL-1α, IL-1β, IL-2, IL-6, and TNFα in vivo; while also secreting higher or similar levels of IL-2. This disclosure provides, inter alio, methods of using antibodies directed to the use of anti-TCRVβ antibodies to expand T cells ex vivo. Use of the anti-TCRβV antibody molecules disclosed herein result in less or no production of cytokines associated with CRS, e.g., IL-6, IL-1beta and TNF alpha; and enhanced and/or delayed production of IL-2 and IFNγ. In some embodiments, the anti-TCRβV antibodies disclosed herein result in expansion of a subset of memory effector T cells known as T_{EMRA}. In some embodiments, the expanded cells are infused into a subject for treatment of a disease (e.g., cancer). In some embodiments, compositions comprising anti-TCRβV antibody molecules of the present disclosure, can be used, e.g., to expand T cells (CAR-T cells) ex vivo to promote tumor cell lysis for cancer immunotherapy. In some embodiments, methods of expanding T cells ex vivo comprising contacting the T cells to an anti-TCRβV antibody molecules as disclosed herein limit the harmful side-effects of CRS, e.g., CRS associated with anti-CD3e targeting and/or CD28 targeting.

### INCORPORATION BY REFERENCE

All publications and patents mentioned herein are hereby incorporated by reference in their entirety as if each individual publication or patent was specifically and individually indicated to be incorporated by reference.

### Definitions

Unless defined otherwise, all technical and scientific terms used herein have the same meaning as commonly understood by one of ordinary skill in the art.

Ranges: throughout this disclosure, various aspects can be presented in a range format. It should be understood that the description in range format is merely for convenience and brevity and should not be construed as an inflexible limitation on the scope. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

The term “a” and “an” refers to one or to more than one (i.e., to at least one) of the grammatical object of the article. By way of example, “an element” means one element or more than one element.

As used herein, the terms “T cell receptor beta variable chain,” “TCRβV,” “TCRβ V,” “TCR βV,” “TCRβv,” “TCR βv,” “TCRβ v,” “T cell receptor variable beta chain,” “TCRβV,” “TCR Vβ,” “TCRV β,” “TCRβV,” “TCRv β,” or “TCR vβ,” are used interchangeably herein and refer to an extracellular region of the T cell receptor beta chain which comprises the antigen recognition domain of the T cell receptor. The term TCRβV includes isoforms, mammalian, e.g., human TCRβV, species homologs of human and analogs comprising at least one common epitope with TCRβV. Human TCRβV comprises a gene family comprising subfamilies including, but not limited to: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβV comprises TCRβ V6-5*01. TCRβ V6-5*01 is also known as TRBV65; TCRβV 6S5; TCRβV 13S1, or TCRβV 13.1. The amino acid sequence of TCRβ V6-5*01, e.g., human TCRβ V6-5*01, is known in that art, e.g., as provided by IMGT ID L36092.

As used herein, the term “molecule” includes full-length, naturally-occurring molecules, as well as variants, e.g., functional variants (e.g., truncations, fragments, mutated (e.g., substantially similar sequences) or derivatized form thereof), so long as at least one function and/or activity of the unmodified (e.g., full length, naturally-occurring) molecule remains.

The terms “antibody,” and “antibody molecule” are used interchangeably herein and refer to a protein comprising at least one immunoglobulin variable domain sequence. The term antibody encompasses full-length antibodies, antibody fragments (e.g., functional fragments thereof), and variants (e.g., functional variants thereof). Antibodies can be polyclonal or monoclonal, multiple or single chain, or intact immunoglobulins, and may be derived from natural sources or from recombinant sources. Antibodies can be tetramers of immunoglobulin molecules. In an embodiment, an antibody molecule comprises an antigen binding or functional fragment of a full length antibody, or a full length immunoglobulin chain. For example, a full-length antibody is an immunoglobulin (Ig) molecule (e.g., an IgG antibody) that is naturally occurring or formed by normal immunoglobulin gene fragment recombinatorial processes). In embodiments, an antibody molecule refers to an immunologically active, antigen-binding portion of an immunoglobulin molecule, such as an antibody fragment. The term “antibody fragment” refers to at least one portion of an intact antibody, or recombinant variants thereof, and refers to the antigen binding domain, e.g., an antigenic determining variable region of an intact antibody, that is sufficient to confer recognition and specific binding of the antibody fragment to a target, such as an antigen. An antibody fragment, e.g., functional fragment, is a portion of an antibody, e.g., Fab, Fab′, F(ab′)₂, F(ab)₂, variable fragment (Fv), domain antibody (dAb), or single chain variable fragment (scFv). A functional antibody fragment binds to the same antigen as that recognized by the intact (e.g., full-length) antibody. The terms “antibody fragment” or “functional fragment” also include isolated fragments consisting of the variable regions, such as the “Fv” fragments consisting of the variable regions of the heavy and light chains or recombinant single chain polypeptide molecules in which light and heavy variable regions are connected by a peptide linker (“scFv proteins”). In some embodiments, an antibody fragment does not include portions of antibodies without antigen binding activity, such as Fc fragments or single amino acid residues. Exemplary antibody molecules include full length antibodies and antibody fragments, e.g., dAb (domain antibody), single chain, Fab, Fab′, and F(ab′)₂ fragments, and single chain variable fragments (scFvs). Examples of antibody fragments include, but are not limited to, Fab, Fab′, F(ab′)₂, and Fv fragments, scFv antibody fragments, linear antibodies, single domain antibodies such as sdAb (either V_{L} or V_{H}), camelid V_{H}H domains, and multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv. Antigen binding fragments can also be grafted into scaffolds based on polypeptides such as a fibronectin type III (Fn3) (see e.g., U.S. Pat. No. 6,703,199, which describes fibronectin polypeptide minibodies, and is incorporated by reference herein). An antigen binding domain can include a nanobody. In some embodiments, the antigen binding domain can be a non-antibody targeting domain. In some embodiments, the antigen binding domain can be a nanobody.

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the V_{L} and V_{H} variable regions in either order, e.g., with respect to the N-terminal and C-terminal ends of the polypeptide, the scFv may comprise V_{L}-linker-V_{H} or may comprise V_{H}-linker-V_{L}.

The terms “complementarity determining region” or “CDR,” are used interchangeably herein and refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273, 927-948 (“Chothia” numbering scheme), or a combination thereof. Under the Kabat numbering scheme, in some embodiments, the CDR amino acid residues in the heavy chain variable domain (V_{H}) are numbered 31-35 (HCDR1), 50-65 (HCDR2), and 95-102 (HCDR3); and the CDR amino acid residues in the light chain variable domain (V_{L}) are numbered 24-34 (LCDR1), 50-56 (LCDR2), and 89-97 (LCDR3). Under the Chothia numbering scheme, in some embodiments, the CDR amino acids in the V_{H} are numbered 26-32 (HCDR1), 52-56 (HCDR2), and 95-102 (HCDR3); and the CDR amino acid residues in the V_{L} are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a V_{H}, e.g., a mammalian V_{H}, e.g., a human V_{H}; and amino acid residues 24-34 (LCDR1), 50-56 (LCDR2), and 89-97 (LCDR3) in a V_{L}, e.g., a mammalian V_{L}, e.g., a human V_{L}.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “specifically binds,” refers to an antibody, or a ligand, which recognizes and binds with a cognate binding partner (e.g., a stimulatory and/or costimulatory molecule present on a T cell) protein present in a sample, but which antibody or ligand does not substantially recognize or bind other molecules in the sample.

As used herein, an “immune cell” refers to any of various cells that function in the immune system, e.g., to protect against agents of infection and foreign matter. In embodiments, this term includes leukocytes, e.g., neutrophils, eosinophils, basophils, lymphocytes, and monocytes. Innate leukocytes include phagocytes (e.g., macrophages, neutrophils, and dendritic cells), mast cells, eosinophils, basophils, and natural killer cells. Innate leukocytes identify and eliminate pathogens, either by attacking larger pathogens through contact or by engulfing and then killing microorganisms, and are mediators in the activation of an adaptive immune response. The cells of the adaptive immune system are special types of leukocytes, called lymphocytes. B cells and T cells are important types of lymphocytes and are derived from hematopoietic stem cells in the bone marrow. B cells are involved in the humoral immune response, whereas T cells are involved in cell-mediated immune response. The term “immune cell” includes immune effector cells.

As used herein the term “immune effector cell,” refers to a cell that is involved in an immune response, e.g., in the promotion of an immune effector response. Examples of immune effector cells include, but are not limited to, T cells (e.g., alpha/beta T cells, gamma/delta T cells CD4+ T cells, CD8+ T cells), B cells, natural killer (NK) cells, natural killer T (NK T) cells, monocytes, macrophages, neutrophils, basophils, dendritic cells and mast cells.

The terms “effector function” or “effector response” refer to a specialized function of a cell. Effector function of a T cell, for example, may be cytolytic activity (e.g., CD8+ T cells) or helper activity (e.g., CD4+ T cells) including the secretion of cytokines.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

The term, a “substantially purified cell” or “substantially purified cell population” refers to a cell or cell population that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature. An isolated nucleic acid or protein can exist in substantially purified form, or can exist in a non-native environment such as, for example, a host cell.

Some compositions and methods described herein encompass polypeptides and nucleic acids having the sequences specified, or sequences substantially identical or similar thereto, e.g., sequences at least 80%, 85%, 90%, 95% identical or higher to the sequence specified. In the context of an amino acid sequence, the term “substantially identical” is used herein to refer to a first amino acid that contains a sufficient or minimum number of amino acid residues that are i) identical to, or ii) conservative substitutions of aligned amino acid residues in a second amino acid sequence such that the first and second amino acid sequences can have a common structural domain and/or common functional activity. For example, amino acid sequences that contain a common structural domain having at least about 80%, 85%, 90%. 91%, 92%, 93%, 94%, 95%, 96%, 97%, 98% or 99% identity to a reference sequence, e.g., a sequence provided herein. In the context of nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity. For example, nucleotide sequences having at least about 80%, 85%, 90%, 91%, 92%, 93%, 94%, 95%, 96%, 97%, 98% or 99% identity to a reference sequence, e.g., a sequence provided herein.

The terms “homology” and “sequence identity” are used interchangeably herein and refer to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous. Calculations of homology between sequences are performed as follows. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences. The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used unless otherwise specified) are a Blossum 62 scoring matrix with a gap penalty of 12, a gap extend penalty of 4, and a frameshift gap penalty of 5. The percent identity between two amino acid or nucleotide sequences can be determined using the algorithm of E. Meyers and W. Miller ((1989) CABIOS, 4:11-17) which has been incorporated into the ALIGN program (version 2.0), using a PAM120 weight residue table, a gap length penalty of 12 and a gap penalty of 4. The nucleic acid and protein sequences described herein can be used as a “query sequence” to perform a search against public databases to, for example, identify other family members or related sequences. Such searches can be performed using the NBLAST and)(BLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to a nucleic acid molecule. BLAST protein searches can be performed with the)(BLAST program, score=50, wordlength=3 to obtain amino acid sequences homologous to protein molecules. To obtain gapped alignments for comparison purposes, Gapped BLAST can be utilized as described in Altschul et al., (1997) Nucleic Acids Res. 25:3389-3402. When utilizing BLAST and Gapped BLAST programs, the default parameters of the respective programs (e.g., XBLAST and NBLAST) can be used. It is understood that the molecules may have additional conservative or non-essential amino acid substitutions, which do not have a substantial effect on their functions.

The term “amino acid” is intended to embrace all molecules, whether natural or synthetic, which include both an amino functionality and an acid functionality and capable of being included in a polymer of naturally-occurring amino acids. Exemplary amino acids include naturally-occurring amino acids; analogs, derivatives and congeners thereof; amino acid analogs having variant side chains; and all stereoisomers of any of any of the foregoing. As used herein the term “amino acid” includes both the D- or L-optical isomers and peptidomimetics.

A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Such conservative modifications include amino acid substitutions, additions and deletions. Modifications can be introduced into an antibody or antibody fragment by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR can be replaced with other amino acid residues from the same side chain family and the altered CAR can be tested using the functional assays described herein.

The terms “polypeptide”, “peptide” and “protein” (if single chain) are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

The terms “nucleic acid,” “nucleic acid sequence,” “nucleotide sequence,” “polynucleotide sequence,” and “polynucleotide” are used interchangeably herein. They refer to a polymeric form of nucleotides of any length, either deoxyribonucleotides or ribonucleotides, or analogs thereof. The polynucleotide may be either single-stranded or double-stranded, and if single-stranded may be the coding strand or non-coding (antisense) strand. A polynucleotide may comprise modified nucleotides, such as methylated nucleotides and nucleotide analogs. The sequence of nucleotides may be interrupted by non-nucleotide components. A polynucleotide may be further modified after polymerization, such as by conjugation with a labeling component. The nucleic acid may be a recombinant polynucleotide, or a polynucleotide of genomic, cDNA, semisynthetic, or synthetic origin which either does not occur in nature or is linked to another polynucleotide in a non-natural arrangement. The following abbreviations for the commonly occurring nucleic acid bases are used. “A” refers to adenosine, “C” refers to cytosine, “G” refers to guanosine, “T” refers to thymidine, and “U” refers to uridine.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “vector” as used herein refers to any vehicle that can be used to deliver and/or express a nucleic acid molecule. It can be a transfer vector or an expression vector as described herein.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive promoter” refers to a nucleotide sequence which, when operably linked with a polynucleotide which encodes or specifies a gene product, causes the gene product to be produced in a cell under most or all physiological conditions of the cell.

The term “inducible promoter” refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific promoter” refers to a nucleotide sequence which, when operably linked with a polynucleotide encodes or specified by a gene, causes the gene product to be produced in a cell substantially only if the cell is a cell of the tissue type corresponding to the promoter.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “chimeric antigen receptor” or alternatively a “CAR” are used interchangeably herein and refer to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, e.g., comprise a chimeric fusion protein. In some embodiments, the domains in the CAR polypeptide construct are not contiguous with each other, e.g., are in different polypeptide chains. In one aspect, the stimulatory molecule of the CAR is the zeta chain associated with the T cell receptor complex. In one aspect, the cytoplasmic signaling domain comprises a primary signaling domain (e.g., a primary signaling domain of CD3-zeta). In one aspect, the cytoplasmic signaling domain further comprises one or more functional signaling domains derived from at least one costimulatory molecule as defined below. In one aspect, the costimulatory molecule is chosen from 4-1BB (i.e., CD137), CD27, ICOS, and/or CD28. In one aspect, the CAR comprises a chimeric fusion protein comprising an extracellular antigen recognition domain, a transmembrane domain and an intracellular signaling domain comprising a functional signaling domain derived from a stimulatory molecule. In one aspect, the CAR comprises a chimeric fusion protein comprising an extracellular antigen recognition domain, a transmembrane domain and an intracellular signaling domain comprising a functional signaling domain derived from a co-stimulatory molecule and a functional signaling domain derived from a stimulatory molecule. In one aspect, the CAR comprises a chimeric fusion protein comprising an extracellular antigen recognition domain, a transmembrane domain and an intracellular signaling domain comprising two functional signaling domains derived from one or more co-stimulatory molecule(s) and a functional signaling domain derived from a stimulatory molecule. In one aspect, the CAR comprises a chimeric fusion protein comprising an extracellular antigen recognition domain, a transmembrane domain and an intracellular signaling domain comprising at least two functional signaling domains derived from one or more co-stimulatory molecule(s) and a functional signaling domain derived from a stimulatory molecule. In one aspect the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., a scFv) during cellular processing and localization of the CAR to the cellular membrane.

The term “signaling domain” as used herein refers to the functional portion of a protein which acts by transmitting information within the cell to regulate cellular activity via defined signaling pathways by generating second messengers or functioning as effectors by responding to such messengers.

An “intracellular signaling domain,” as the term is used herein, refers to an intracellular portion of a molecule. The intracellular signaling domain can generate a signal that promotes an immune effector function of the CAR containing cell, e.g., a CART cell or CAR-expressing NK cell. Examples of immune effector function, e.g., in a CART cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal. In some embodiment, the intracellular signaling domain comprises a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CAR-expressing immune effector cell, e.g., CART cell or CAR-expressing NK cell, a primary intracellular signaling domain can comprise a cytoplasmic sequence of a T cell receptor, and a costimulatory intracellular signaling domain can comprise cytoplasmic sequence from co-receptor or costimulatory molecule. A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CDS, CD22, CD79a, CD79b, CD278 (“ICOS”), FcεRI, CD66d, DAP10, and DAP12.

The term “zeta” or alternatively “zeta chain”, “CD3-zeta” or “TCR-zeta” is defined as the protein provided as GenBan Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an a MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, V_{L}A1, CD49a, ITGA4, IA4, CD49D, ITGA6, V_{L}A-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A “costimulatory intracellular signaling domain” refers to the intracellular portion of a costimulatory molecule. The intracellular signaling domain can comprise the entire intracellular portion, or the entire native intracellular signaling domain, of the molecule from which it is derived, or a functional fragment thereof.

The term “signal transduction pathway” as used herein refers to the biochemical relationship between a variety of signal transduction molecules that play a role in the transmission of a signal from one portion of a cell to another portion of a cell.

The term “cell surface receptor” as used herein includes molecules and complexes of molecules capable of receiving a signal and transmitting signal across the membrane of a cell.

The term “anti-tumor effect” or “anti-cancer effect,” used interchangeably herein refer to a biological effect which can be manifested by various means, including but not limited to, e.g., a decrease in tumor volume, a decrease in the number of tumor cells, a decrease in the number of metastases, an increase in life expectancy, decrease in tumor cell proliferation, decrease in tumor cell survival, or amelioration of various physiological symptoms associated with the cancerous condition. An “anti-tumor effect” can also be manifested by the ability of the peptides, polynucleotides, cells and antibodies in prevention of the occurrence of tumor in the first place.

The terms “Cancer” or “tumor” as used interchangeably herein and encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer. For example, both terms encompass solid and liquid tumors. As used herein, the term cancer includes premalignant, as well as malignant cancers and tumors.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, e.g., by retransfusion. Thus, in the context of “an apheresis sample” refers to a sample obtained using apheresis.

The term “combination” refers to either a fixed combination in one dosage unit form, or a combined administration where a compound and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both of the components (e.g., powders or liquids) may be reconstituted or diluted to a desired dose prior to administration. The terms “co-administration” or “combined administration” or the like as utilized herein are meant to encompass administration of the selected combination partner to a single subject in need thereof (e.g. a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g. a compound and a combination partner, are both administered to a patient simultaneously in the form of a single entity or dosage. The term “non-fixed combination” means that the active ingredients, e.g. a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g. the administration of three or more active ingredients.

The term “effective amount” or “therapeutically effective amount” are used interchangeably herein, and refer to an amount of a compound, formulation, material, or composition, as described herein effective to achieve a particular biological result.

As used herein, the terms “treat,” “treatment,” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as a CAR). In specific embodiments, the terms “treat,” “treatment,” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat,” “treatment,” and “treating” refer to the reduction or stabilization of tumor size or cancerous cell count.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “subject” is intended to include living organisms in which an immune response can be elicited (e.g., mammals, human).

### Sources and Isolation of T Cells

In some aspects described herein, prior to activation and expansion, T cells are obtained from a subject (e.g., a human subject). Examples of subjects include humans, dogs, cats, mice, rats, and transgenic species thereof. T cells can be obtained from a number of sources, including but not limited to, blood, peripheral blood mononuclear cells (PBMCs), bone marrow, lymph node tissue, cord blood, thymus tissue, tissue from a site of infection, ascites, pleural effusion, spleen tissue, and tumors. In some embodiments, T cells are obtained from a unit of blood collected from a subject using any number of techniques known to the skilled artisan, such as ficoll separation.

In some embodiments, cells from the circulating blood of an individual are obtained by apheresis or leukapheresis. The apheresis product can contain lymphocytes, including T cells, monocytes, granulocytes, B cells, other nucleated white blood cells, red blood cells, and platelets. In some embodiments, the cells collected by apheresis are washed to remove the plasma fraction and to place the cells in an appropriate buffer or media for subsequent processing steps. In some embodiments, the cells are washed with phosphate buffered saline (PBS). In some embodiments, the wash solution lacks calcium, lacks magnesium, lacks both calcium and magnesium, or lacks all divalent cations. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. In some embodiments, after washing, the cells are resuspended in a variety of biocompatible buffers, for example, Ca-free, Mg-free PBS, PlasmaLyte A, or other saline solution with or without buffer. Alternatively, in some embodiments, the undesirable components of the apheresis sample are removed and the cells directly resuspended in culture media.

Collected apheresis products can be processed in various ways depending on the downstream procedures. Devices such as Haemonetics Cell Saver 5+, COBE2991, and Fresenius Kabi LOVO have the ability to remove gross red blood cells and platelet contaminants. Terumo Elutra and Biosafe Sepax systems provide size-based cell fractionation for the depletion of monocytes and the isolation of lymphocytes. Instruments such as CliniMACS Plus and Prodigy systems allow the enrichment of specific subsets of T cells, such as CD4⁺, CD8⁺, CD25⁺, or CD62L⁺ T cells using Miltenyi beads post-cell washing.

Enrichment of a T cell population by negative selection can be accomplished using a combination of antibodies directed to surface markers unique to the negatively selected cells. For example, one method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8. In certain embodiments, it may be desirable to enrich for or positively select for regulatory T cells which typically express CD4+, CD25+, CD62Lhi, GITR+, and FoxP3+. Alternatively, in certain embodiments, T regulatory cells are depleted by anti-CD25 conjugated beads or other similar method of selection.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, monocyte populations (i.e., CD14+ cells) are depleted from blood preparations prior to ex vivo expansion by a variety of methodologies, including anti-CD14 coated beads or columns, or utilization of the phagocytotic activity of these cells to facilitate removal, or by the use of counterflow centrifugal elutriation. In certain embodiments, paramagnetic particles of a size sufficient to be engulfed by phagocytotic monocytes are used. In certain embodiments, the paramagnetic particles are commercially available beads, for example, those produced by Dynal AS under the trade name Dynabeads™. Exemplary Dynabeads™ in this regard are M-280, M-450, and M-500. In some embodiments, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be expanded. In certain embodiments the irrelevant beads include beads coated with sheep anti-mouse antibodies, goat anti-mouse antibodies, and human serum albumin.

In some embodiments, T cells are obtained from a patient directly following a therapeutic agent (e.g., an agent administered to a subject to treat cancer). In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context to collect blood cells, including T cells, dendritic cells, or other cells of the hematopoietic lineage, during this recovery phase. Further, in certain embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, T cells are cultured ex vivo on a biocompatible substantially non-toxic surface. In some embodiments, the surface comprises agent/or ligands that bind to the surface. The biocompatible surface may be biodegradable or non-biodegradable. The surface may be natural or synthetic (e.g., a polymer).

In some embodiments, an agent is attached or coupled to, or integrated into a surface by a variety of methods known and available in the art. In some embodiments, the agent is a natural ligand, a protein ligand, or a synthetic ligand. The attachment may be covalent or noncovalent, electrostatic, or hydrophobic and may be accomplished by a variety of attachment means, including for example, chemical, mechanical, enzymatic, electrostatic, or other means whereby a ligand is capable of stimulating the cells. For example, the antibody to a ligand first may be attached to a surface, or avidin or streptavidin may be attached to the surface for binding to a biotinylated ligand. The antibody to the ligand may be attached to the surface via an anti-idiotype antibody. Another example includes using protein A or protein G, or other non-specific antibody binding molecules, attached to surfaces to bind an antibody. Alternatively, the ligand may be attached to the surface by chemical means, such as cross-linking to the surface, using commercially available cross-linking reagents (Pierce, Rockford, Ill.) or other means. In certain embodiments, the ligands are covalently bound to the surface.

In some embodiments, the agent, such as certain ligands are of singular origin or multiple origins. In some embodiments, the agent is an antibody or functional fragment thereof. Furthermore, one of ordinary skill in the art will recognize that any ligand useful in the activation and induction of proliferation of a subset of T cells may also be immobilized on the surface of the biocompatible substance. In addition, while covalent binding of the ligand to the surface is one preferred methodology, adsorption or capture by a secondary monoclonal antibody may also be used. The amount of a particular ligand attached to a surface may be readily determined by flow cytometric analysis if the surface is that of beads or determined by enzyme-linked immunosorbent assay (ELISA) if the surface is a tissue culture dish, mesh, fibers, bags, for example.

In some embodiments, blood samples or leukapheresis products are collected from a subject at a time period prior to when the expanded cells as described herein are needed. As such, the source of the cells to be expanded can be collected at any time point necessary, and desired cells, such as T cells, isolated and frozen for later use in T cell therapy for any number of diseases or conditions that would benefit from T cell therapy, such as those described herein. In one embodiment a blood sample or a leukapheresis is taken from a generally healthy subject. In certain embodiments, a blood sample or a leukapheresis is taken from a generally healthy subject who is at risk of developing a disease, but who has not yet developed a disease, and the cells of interest are isolated and frozen for later use. In certain embodiments, the T cells may be expanded, frozen, and used at a later time. In certain embodiments, samples are collected from a patient shortly after diagnosis of a particular disease as described herein but prior to any treatments. In a further embodiment, the cells are isolated from a blood sample or a leukapheresis from a subject prior to any number of relevant treatment modalities, including but not limited to treatment with agents such as antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxin, fludaribine, cyclosporin, FK506, rapamycin, mycophenolic acid, steroids, FR901228, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cells are isolated for a patient and frozen for later use in conjunction with (e.g. before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cells are isolated prior to and can be frozen for later use for treatment following B-cell ablative therapy such as agents that react with CD20, e.g. Rituxan.

In some embodiments, following isolation, T cells are incubated in cell medium in a culture apparatus for a period of time or until the cells reach confluency before passing the cells to another culture apparatus. The culturing apparatus can be of any culture apparatus commonly used for culturing cells in vitro. A period of time can be any time suitable for the culture of cells in vitro. T cell medium may be replaced during the culture of the T cells at any time. In some embodiments, the T cell medium is replaced about every 2 to 3 days. In some embodiments, T cells are then harvested from the culture apparatus whereupon the T cells can be used immediately or cryopreserved to be stored for use at a later time. In some embodiments, T cells are harvested by trypsinization, EDTA treatment, or any other procedure used to harvest cells from a culture apparatus.

### Activating and Expanding T Cells

Provided herein are, inter alia, methods of activating and expanding T cells ex vivo. In some embodiments, the methods comprise expanding T cells ex vivo using an anti-TCRβV agent, e.g., an anti-TCRβV antibody or functional fragment or functional variant thereof. Accordingly, in some embodiments, the methods described herein allow for activation and expansion of any T cell population ex vivo and substantially increasing the number of T cells for subsequent use following expansion. Accordingly, in some aspects, provided herein are methods of multiplying, expanding or otherwise culturing T cells isolated from a subject ex vivo, using the methods disclosed herein.

In some embodiments, the anti-TCRβV agent, e.g., anti-TCRβV antibody, is coupled to a solid surface, e.g., a bead, a cell culture plate, etc.

In some embodiments, at least a plurality of the T cells being expanded comprise an exogenous nucleic acid or polypeptide. In some embodiments, the exogenous nucleic acid encodes a chimeric polypeptide. In some embodiments, the exogenous nucleic acid encodes an exogenous polypeptide. In some embodiments, the chimeric polypeptide encodes a chimeric antigen receptor or a chimeric T cell receptor. In some embodiments, the exogenous nucleic acid encodes an exogenous cellular receptor. In some embodiments, said exogenous cellular receptor is an exogenous T cell receptor. In some embodiments, the polypeptide comprises a chimeric antigen receptor or a chimeric T cell receptor. In some embodiments, the polypeptide comprises an exogenous cellular receptor. In some embodiments, said exogenous cellular receptor is an exogenous T cell receptor.

In some embodiments, the methods described herein comprise introducing an exogenous nucleic acid into a plurality of T cells prior to contacting the plurality of T cells with the anti-TCRβV agent, e.g., anti-TCRβV antibody. In some embodiments, the methods described herein comprise introducing an exogenous nucleic acid into a plurality of T cells after contacting the plurality of T cells with anti-TCRβV agent, e.g., anti-TCRβV antibody. In some embodiments, the methods described herein comprise contacting a plurality of T cells with the anti-TCRβV agent, e.g., anti-TCRβV antibody, then introducing an exogenous nucleic acid into the plurality of T cells while continuing to contact the plurality of T cells with the anti-TCRβV agent, e.g., anti-TCRβV antibody. In some embodiments, the exogenous nucleic acid encodes a chimeric antigen receptor (CAR). In some embodiments, the exogenous nucleic acid encodes a T cell receptor.

In some embodiments, methods of expanding T cells ex vivo comprise contacting a plurality of T cells with a first agent, wherein the first agent comprises a domain that specifically binds to a TCRβV region, thereby generating a first population of T cells. In some embodiments, the first population of T cells exhibit one or more of: reduced expression of IL-1β, reduced expression level of IL-6, reduced expression of TNFα, increased expression of IL-2, reduced expression of IFNγ, relative to a plurality of T cells contacted with an agent comprising a domain that specifically binds CD3ε.

In some embodiments, the contacting comprises incubating or culturing the plurality of T cells with an anti-TCRβV antibody (e.g., as described herein) for at least about 10 minutes, 20 minutes, 30 minutes, 1 hour, 6 hours, 10 hours, 12 hours, 24 hours, 36 hours, 48 hours, 72 hours, 5 days, 7 days, 10 days, 14 days, 15 days, or 30 days. In some embodiments, contacting comprises incubating or culturing the plurality of T cells with an anti-TCRβV antibody (e.g., as described herein) for at most about 10 minutes, 20 minutes, 30 minutes, 1 hour, 6 hours, 10 hours, 12 hours, 24 hours, 36 hours, 48 hours, 72 hours, 4 days, 5 days, 6 days, 7 days, 8 days, 9 days, 10 days, 11 days, 12 days, 13 days, 14 days, 15 days, 21 days, 30 days, 45 days, or 60 days. In some embodiments, contacting comprises incubating or culturing the plurality of T cells with an anti-TCRβV antibody (e.g., as described herein) for about from 10-60 minutes, 10-30 minutes, 1-30 days, 1-21 days, 1-14 days, 1-10 days, 1-9 days, 1-8 days, 1-7 days, 1-6 days, 1-5 days, 1-4 days, 1-3 days, 1-2 days, 21-30 days, 14-30 days, 7-30 days, 5-30 days, or 3-30 days.

In some embodiments, methods of activating or expanding T cells comprises contacting a plurality of T cells to a plurality of with a plurality of anti-TCRβV antibodies (e.g., as described herein), wherein the plurality of agents comprises at least two, three, four, five, six, seven, eight, nine, or ten agents, wherein each anti-TCRβV antibody of the plurality comprises a domain that specifically binds to a different TCRβV region, thereby generating a first population of T cells. In some embodiments, each anti-TCRβV antibody of the plurality specifically binds to a different TCRβV, wherein each TCRβV belongs to a different TCRβV subfamily or are different members of the same TCRβV subfamily. In some embodiments, each anti-TCRβV antibody of the plurality comprises a domain that specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, each agent of the plurality specifically binds to a different TCRβV, wherein each TCRβV belongs to a different TCRβV subfamily.

In some embodiments, the first anti-TCRβV antibody further comprises a second domain that binds to a protein expressed on the surface of a population of T cells in the plurality. In some embodiments, the first anti-TCRβV antibody is a bispecific antibody molecule. In some embodiments, the second domain specifically binds to a TCRβV region. In some embodiments, the second domain and the first domain specifically bind different TCRβV regions. In some embodiments, the second domain and the first domain specifically bind TCRβVs belonging to different subfamilies or different members of the same TCRβV subfamily. In some embodiments, the first domain specifically binds specifically binds to a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily, and the second domain specifically binds a TCRβV region of a TCRβV belonging to a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the second domain and the first domain specifically bind TCRβVs belonging to different subfamilies. In some embodiments, the second domain and the first domain specifically bind different members of the same TCRβV subfamily. In some embodiments, the second domain specifically binds to CD19 or 4-1BB.

### Human T Cell Receptor (TCR) Complex

T cell receptors (TCR) are expressed on the surface of T cells. TCRs recognize antigens, e.g., peptides, presented on, e.g., bound to, major histocompatibility complex (MHC) molecules on the surface of cells, e.g., antigen-presenting cells. TCRs are heterodimeric molecules and can comprise an alpha chain, a beta chain, a gamma chain or a delta chain. TCRs comprising an alpha chain and a beta chain are also referred to as TCRαβ. The TCR beta chain consists of the following regions (also known as segments): variable (V), diversity (D), joining (J) and constant (C) (see Mayer G. and Nyland J. (2010) Chapter 10: Major Histocompatibility Complex and T-cell Receptors-Role in Immune Responses. In: Microbiology and Immunology on-line, University of South Carolina School of Medicine). The TCR alpha chain consists of V, J and C regions. The rearrangement of the T-cell receptor (TCR) through somatic recombination of V (variable), D (diversity), J (joining), and C (constant) regions is a defining event in the development and maturation of a T cell. TCR gene rearrangement takes place in the thymus.

TCRs can comprise a receptor complex, known as the TCR complex, which comprises a TCR heterodimer comprising of an alpha chain and a beta chain, and dimeric signaling molecules, e.g., CD3 co-receptors, e.g., CD3δ/ε, and/or CD3γ/ε.

### TCRβV

Diversity in the immune system enables protection against a huge array of pathogens. Since the germline genome is limited in size, diversity is achieved not only by the process of V(D)J recombination but also by junctional (junctions between V-D and D-J segments) deletion of nucleotides and addition of pseudo-random, non-templated nucleotides. The TCR beta gene undergoes gene arrangement to generate diversity.

The TCR V beta repertoire varies between individuals and populations because of, e.g., 7 frequently occurring inactivating polymorphisms in functional gene segments and a large insertion/deletion-related polymorphism encompassing 2 V beta gene segments.

This disclosure provides, inter alia, antibody molecules and functional fragments thereof, that bind, e.g., specifically bind, to a human TCR beta V chain (TCRβV), e.g., a TCRβV gene family, e.g., a TCRβV subfamily, e.g., as described herein. TCR beta V families and subfamilies are known in the art, e.g., as described in Yassai et al., (2009) Immunogenetics 61(7) pp:493-502; Wei S. and Concannon P. (1994) Human Immunology 41(3) pp: 201-206. The antibodies described herein can be recombinant antibodies, e.g., recombinant non-murine antibodies, e.g., recombinant human or humanized antibodies.

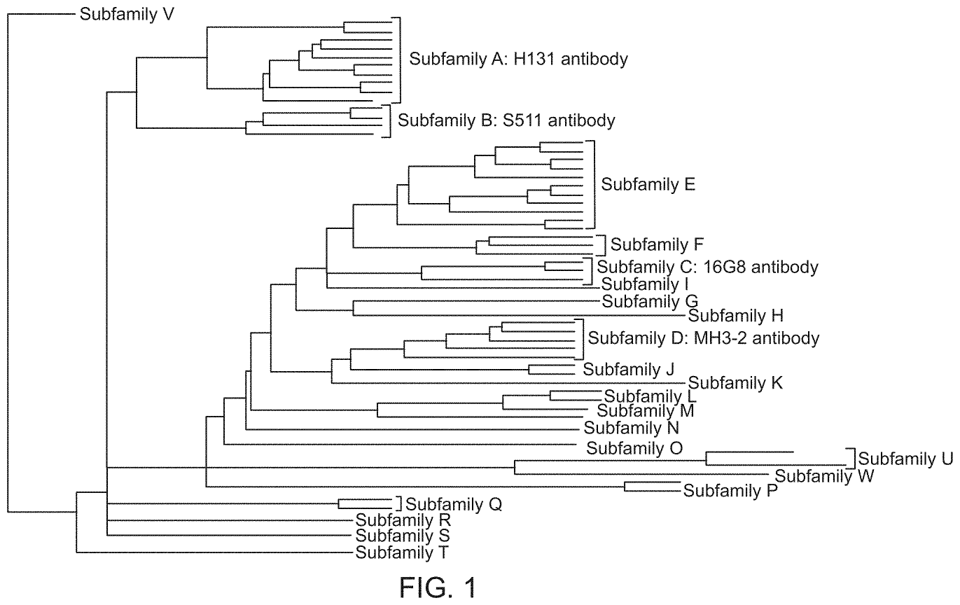

In an aspect, the disclosure provides an anti-TCRβV antibody molecule that binds to human TCRβV, e.g., a TCRβV family, e.g., gene family. In some embodiments a TCRβV gene family comprises one or more subfamilies, e.g., as described herein, e.g., in FIG. 1. In some embodiments, the TCRβV gene family comprises subfamilies comprising: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily.

The TCRβ V6 subfamily is also known as TCRβ V13.1. In some embodiments, the TCRβ V6 subfamily comprises: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, TCRβ V6 comprises TCRβ V6-5*01. In some embodiments, TCRβ V6, e.g., TCRβ V6-5*01, is recognized, e.g., bound, by SEQ ID NO: 11 and/or SEQ ID NO: 10.

The TCRβ V10 subfamily is also known as TCRβ V12. In some embodiments, the TCRβ V10 subfamily comprises: TCRβ V10-1*01, TCRβ V10-1*02, TCRβ V10-3*01 or TCRβ V10-2*01.

The TCRβ V12 subfamily is also known as TCRβ V8.1. In some embodiments, the TCRβ V12 subfamily comprises: TCRβ V12-4*01, TCRβ V12-3*01, or TCRβ V12-5*01. In some embodiments, TCRβ V12 is recognized, e.g., bound, by SEQ ID NO: 58

In some embodiments, the TCRβ V5 subfamily is chosen from: TCRβ V5-5*01, TCRβ V5-6*01, TCRβ V5-4*01, TCRβ V5-8*01, TCRβ V5-1*01. In some embodiments, the TCRβ V7 subfamily comprises TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01. In some embodiments, the TCRβ V11 subfamily comprises: TCRβ V11-1*01, TCRβ V11-2*01 or TCRβ V11-3*01.

In some embodiments, the TCRβ V14 subfamily comprises TCRβ V14*01. In some embodiments, the TCRβ V16 subfamily comprises TCRβ V16*01. In some embodiments, the TCRβ V18 subfamily comprises TCRβ V18*01. In some embodiments, the TCRβ V9 subfamily comprises TCRβ V9*01 or TCRβ V9*02. In some embodiments, the TCRβ V13 subfamily comprises TCRβ V13*01. In some embodiments, the TCRβ V4 subfamily comprises TCRβ V4-2*01, TCRβ V4-3*01, or TCRβ V4-1*01. In some embodiments, the TCRβ V3 subfamily comprises TCRβV3-1*01. In some embodiments, the TCRβ V2 subfamily comprises TCRβ V2*01. In some embodiments, the TCRβ V15 subfamily comprises TCRβ V15*01. In some embodiments, the TCRβ V30 subfamily comprises TCRβ V30*01, or TCRβ V30*02. In some embodiments, the TCRβ V19 subfamily comprises TCRβ V19*01, or TCRβ V19*02. In some embodiments, the TCRβ V27 subfamily comprises TCRβV27*01. In some embodiments, the TCRβ V28 subfamily comprises TCRβ V28*01. In some embodiments, the TCRβ V24 subfamily comprises TCRβ V24-1*01. In some embodiments, the TCRβ V20 subfamily comprises TCRβ V20-1*01, or TCRβ V20-1*02. In some embodiments, the TCRβ V25 subfamily comprises TCRβ V25-1*01. In some embodiments, the TCRβ V29 subfamily comprises TCRβ V29-1*01.

TABLE 1
List of TCRβV subfamilies and subfamily members

| Reference | | |
|---|---|---|
| in FIG. 1 | Subfamily | Subfamily members |
| A | TCRβ V6 | TCRβ V6-4*01, TCRβ V6-4*02, |
| | Also referred to as: | TCRβ V6-9*01, TCRβ V6-8*01, |
| | TCR VB 13.1 | TCRβ V6-5*01, TCRβ V6-6*02, |
| | | TCRβ V6-6*01, TCRβ V6-2*01, |
| | | TCRβ V6-3*01 or TCRβ V6-1*01. |
| B | TCRβ V10 | TCRβ V10-1*01, TCRβ V10-1*02, |
| | Also referred to as: | TCRβ V10-3*01 or TCRβ V10-2*01 |
| | TCRβ V12 | |
| C | TCRβ V12 | TCRβ V12-4*01, TCRβ V12-3*01, |
| | Also referred to as: | or TCRβ V12-5*01 |
| | TCRβ V8.1 | |
| D | TCRβ V5 | TCRβ V5-5*01, TCRβ V5-6*01, |
| | | TCRβ V5-4*01, TCRβ V5-8*01, |
| | | TCRβ V5-1*01 |
| E | TCRβ V7 | TCRβ V7-7*01, TCRβ V7-6*01, |
| | | TCRβ V7 -8*02, TCRβ V7 -4*01, |
| | | TCRβ V7-2*02, TCRβ V7-2*03, |
| | | TCRβ V7-2*01, TCRβ V7-3*01, |
| | | TCRβ V7-9*03, or TCRβ V7-9*01 |
| F | TCRβ V11 | TCRβ V11-1*01, TCRβ V11-2*01 |
| | | or TCRβ V11-3*01 |
| G | TCRβ V14 | TCRβ V14*01 |
| H | TCRβ V16 | TCRβ V16*01 |
| I | TCRβ V18 | TCRβ V18*01 |
| J | TCRβ V9 | TCRβ V9*01 or TCRβ V9*02 |
| K | TCRβ V13 | TCRβ V13*01 |
| L | TCRβ V4 | TCRβ V4-2*01, TCRβ V4-3*01, |
| | | or TCRβ V4-1*01 |
| M | TCRβ V3 | TCRβ V3-1*01 |
| N | TCRβ V2 | TCRβ V2*01 |
| O | TCRβ V15 | TCRβ V15*01 |
| P | TCRβ V30 | TCRβ V30*01, or TCRβ V30*02 |
| Q | TCRβ V19 | TCRβ V19*01, or TCRβ V19*02 |
| R | TCRβ V27 | TCRβ V27*01. |
| S | TCRβ V28 | TCRβ V28*01. |
| T | TCRβ V24 | TCRβ V24-1*01 |
| U | TCRβ V20 | TCRβ V20-1*01, or TCRβ V20-1*02 |
| V | TCRβ V25 | TCRβ V25-1*01 |
| W | TCRβ V29 | TCRβ V29-1*01 |

### Anti-TCRβV Antibodies

In some embodiments, methods provided herein comprise contacting a population of T cells ex vivo to at least one anti-TCRβV antibody molecule that binds to human TCRβV, e.g., a TCRβV gene family, e.g., one or more of a TCRβV subfamily, e.g., as described herein, e.g., in FIG. 1; Table 1. In some embodiments, an anti-TCRβV antibody described herein that binds to a human TCRβV protein of a family or subfamily disclosed in Table 1. In some embodiments, the anti-TCRβV antibody molecule binds to one or more TCRβV subfamilies chosen from: a TCRβ V6 subfamily, a TCRβ V10 subfamily, a TCRβ V12 subfamily, a TCRβ V5 subfamily, a TCRβ V7 subfamily, a TCRβ V11 subfamily, a TCRβ V14 subfamily, a TCRβ V16 subfamily, a TCRβ V18 subfamily, a TCRβ V9 subfamily, a TCRβ V13 subfamily, a TCRβ V4 subfamily, a TCRβ V3 subfamily, a TCRβ V2 subfamily, a TCRβ V15 subfamily, a TCRβ V30 subfamily, a TCRβ V19 subfamily, a TCRβ V27 subfamily, a TCRβ V28 subfamily, a TCRβ V24 subfamily, a TCRβ V20 subfamily, TCRβ V25 subfamily, or a TCRβ V29 subfamily. In some embodiments, the anti-TCRβV antibody molecule binds to a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01. In some embodiments, the anti-TCRβV antibody molecule binds to a TCRβ V10 subfamily comprising: TCRβ V10-1*01, TCRβ V10-1*02, TCRβ V10-3*01 or TCRβ V10-2*01. In some embodiments, the anti-TCRβV antibody molecule binds to a TCRβ V12 subfamily comprising: TCRβ V12-4*01, TCRβ V12-3*01 or TCRβ V12-5*01. In some embodiments, the anti-TCRβV antibody molecule binds to a TCRβ V5 subfamily comprising: TCRβ V5-5*01, TCRβ V5-6*01, TCRβ V5-4*01, TCRβ V5-8*01, TCRβ V5-1*01.

In some embodiments, the anti-TCRβV antibody binds to at least two TCRβV subfamilies of a Subfamily in Table 1. For example, in some embodiments, the anti-TCRβV antibody binds at least two (e.g., at least 3, 4, 5, or 6) of TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments, the anti-TCRβV antibody binds at least two (e.g., at least 3, 4, 5, or 6) of TCRβ V10-1*01, TCRβ V10-1*02, TCRβ V10-3*01 or TCRβ V10-2*01. In some embodiments, the anti-TCRβV antibody binds at least two (e.g., at least 3, 4, 5, or 6) of TCRβ V12-4*01, TCRβ V12-3*01, or TCRβ V12-5*01. In some embodiments, the anti-TCRβV antibody binds at least two (e.g., at least 3, 4, 5, or 6) of TCRβ V5-5*01, TCRβ V5-6*01, TCRβ V5-4*01, TCRβ V5-8*01, TCRβ V5-1*01. In some embodiments, the anti-TCRβV antibody binds at least two (e.g., at least 3, 4, 5, or 6) of TCRβ V7-7*01, TCRβ V7-6*01, TCRβ V7-8*02, TCRβ V7-4*01, TCRβ V7-2*02, TCRβ V7-2*03, TCRβ V7-2*01, TCRβ V7-3*01, TCRβ V7-9*03, or TCRβ V7-9*01. In some embodiments, the anti-TCRβV antibody binds at least two (e.g., at least 3, 4, 5, or 6) of TCRβ V11-1*01, TCRβ V11-2*01 or TCRβ V11-3*01. In some embodiments, the anti-TCRβV antibody binds at least two of TCRβ V9*01 or TCRβ V9*02. In some embodiments, the anti-TCRβV antibody binds at least two (e.g., at least 3) of TCRβ V4-2*01, TCRβ V4-3*01, or TCRβ V4-1*01. In some embodiments, the anti-TCRβV antibody binds at least two of TCRβ V30*01, or TCRβ V30*02. In some embodiments, the anti-TCRβV antibody binds at least two of TCRβ V19*01, or TCRβ V19*02. In some embodiments, the anti-TCRβV antibody binds at least two of TCRβ V20-1*01, or TCRβ V20-1*02.

In some embodiments, the anti-TCRβV antibody binds at least two different subfamilies of TCRVB. For example, in some embodiments, anti-TCRβV antibody binds a first TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily, and the second domain specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily; and also binds to a second TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily, and the second domain specifically binds to a TCRβV region of a TCRβV belonging to a TCRβV6 subfamily, a TCRβV10 subfamily, a TCRβV12 subfamily, a TCRβV5 subfamily, a TCRβV7 subfamily, a TCRβV11 subfamily, a TCRβV14 subfamily, a TCRβV16 subfamily, a TCRβV18 subfamily, a TCRβV9 subfamily, a TCRβV13 subfamily, a TCRβV4 subfamily, a TCRβV3 subfamily, a TCRβV2 subfamily, a TCRβV15 subfamily, a TCRβV30 subfamily, a TCRβV19 subfamily, a TCRβV27 subfamily, a TCRβV28 subfamily, a TCRβV24 subfamily, a TCRβV20 subfamily, TCRβV25 subfamily, or a TCRβV29 subfamily; wherein the first and second TCRβV regions belong to different TCRβV subfamilies (e.g., TCRβV 5 subfamily and TCRβV 7 subfamily.

In some embodiments, the anti-TCRβV antibody comprises an antibody sequence, e.g., CDRs, VH, VL, humanized VH and humanized VL chain sequences, disclosed in US20180256716, the contents of which are hereby incorporated by reference herein in their entirety.

In some embodiments, the anti-TCRβV antibody is an idiotypic antibody. In some embodiments, the anti-TCRβV antibody is a human antibody. In some embodiments, the anti-TCRβV antibody is a murine antibody. In some embodiments, the anti-TCRβV antibody is a humanized antibody. In some embodiments, the anti-TCRβV antibody is a single chain Fv (scFv) or a Fab. In some embodiments, the anti-TCRβV antibody is a full antibody comprising two antibody heavy chains, each heavy chain comprising a variable region and a constant region; and two antibody light chains, each light chain comprising a variable region and a constant region.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155, which is incorporated by reference herein.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155, which is incorporated by reference herein.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155, which is incorporated by reference herein.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155, which is incorporated by reference herein.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155, which is incorporated by reference herein.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155, which is incorporated by reference herein.

In one embodiment, the first anti-TCRβV antibody is an anti-TCRβ V6 antibody. In some embodiments, the anti-TCRβV antibody molecule binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01. In some embodiments, the anti-TCRβ V6 (e.g., anti-TCRβ V6-5*01) antibody, includes at least one, two, or three complementarity determining regions (CDRs) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences. In some embodiments, the anti-TCRβ V6 (e.g., anti-TCRβ V6-5*01) antibody, includes at least one, two, or three CDRs (or collectively all of the CDRs) from a light chain variable region comprising an amino acid sequence shown in Table 2, or encoded by a nucleotide sequence shown in Table 2. In one embodiment, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβ V6 (e.g., anti-TCRβ V6-5*01) antibody comprises a heavy chain (HC) having a CDR1, a CDR2, and a CDR3; wherein the CDR1, CDR2 and CDR3 has a sequence as set forth in SEQ ID NO: 1, SEQ ID NO: 2, and SEQ ID NO: 3 respectively, according to Combined CDR1, CDR2, and CDR3 definition. In some embodiments, the anti-TCRβ V6 (e.g., anti-TCRβ V6-5*01) antibody molecule comprises a heavy chain (HC) having a CDR1, a CDR2, and a CDR3; wherein the CDR1, CDR2 and CDR3 has a sequence as set forth in SEQ ID NO: 4, SEQ ID NO: 2, and SEQ ID NO: 3 respectively, according to Kabat CDR1, CDR2, and CDR3 definition. In some embodiments, the anti-TCRβ V6 (e.g., anti-TCRβ V6-5*01) antibody molecule comprises a heavy chain (HC) having a CDR1, a CDR2, and a CDR3; wherein the CDR1, CDR2 and CDR3 has a sequence as set forth in SEQ ID NO: 5, SEQ ID NO: 6, and SEQ ID NO: 3 respectively, according to Chothia CDR1, CDR2, and CDR3 definition.

In some embodiments, the anti-TCRβ V6 (e.g., anti-TCRβ V6-5*01) antibody comprises a light chain (LC) having a CDR1, a CDR2, and a CDR3; wherein the CDR1, CDR2 and CDR3 has a sequence as set forth in SEQ ID NO: 7, SEQ ID NO: 8, and SEQ ID NO: 9 respectively, according to Combined CDR1, CDR2, and CDR3 definition. In some embodiments, the anti-TCRβ V6 (e.g., anti-TCRβ V6-5*01) antibody molecule comprises a light chain (LC) having a CDR1, a CDR2, and a CDR3; wherein the CDR1, CDR2 and CDR3 has a sequence as set forth in SEQ ID NO: 7, SEQ ID NO: 8, and SEQ ID NO: 9 respectively according to Kabat CDR1, CDR2, and CDR3 definition. In some embodiments, the anti-TCRβ V6 (e.g., anti-TCRβ V6-5*01) antibody comprises a light chain (LC) having a CDR1, a CDR2, and a CDR3; wherein the CDR1, CDR2 and CDR3 has a sequence as set forth in SEQ ID NO: 7, SEQ ID NO: 8, and SEQ ID NO: 9 respectively, according to Chothia CDR1, CDR2, and CDR3 definition. In some embodiments, the anti-TCRβ V6 (e.g., anti-TCRβ V6-5*01) antibody comprises a heavy chain (HC) variable region (VH) as set forth in SEQ ID NO: 12, or a sequence substantially identical (e.g., at least 80%, 85%, 90%, 92%, 95%, 97%, 98%, 99% or higher identical) to SEQ ID NO: 12. In some embodiments, the anti-TCRβ V6 (e.g., anti-TCRβ V6-5*01) antibody molecule comprises a light chain (LC) variable region (VL) as set forth in SEQ ID NO: 13, or a sequence substantially identical (e.g., at least 80%, 85%, 90%, 92%, 95%, 97%, 98%, 99% or higher identical) to SEQ ID NO: 13. In some embodiments, the anti-TCRβ V6 (e.g., anti-TCRβ V6-5*01) antibody molecule comprises a heavy chain (HC) variable region (VH) as set forth in SEQ ID NO: 14, or a sequence substantially identical (e.g., at least 80%, 85%, 90%, 92%, 95%, 97%, 98%, 99% or higher identical) to SEQ ID NO: 14. In some embodiments, the anti-TCRβ V6 (e.g., anti-TCRβ V6-5*01) antibody molecule comprises a heavy chain (HC) variable region (VH) as set forth in SEQ ID NO: 15, or a sequence substantially identical (e.g., at least 80%, 85%, 90%, 92%, 95%, 97%, 98%, 99% or higher identical) to SEQ ID NO: 15. In some embodiments, the anti-TCRβ V6 (e.g., anti-TCRβ V6-5*01) antibody molecule comprises a heavy chain (HC) variable region (VH) as set forth in SEQ ID NO: 12, or a sequence substantially identical (e.g., at least 80%, 85%, 90%, 92%, 95%, 97%, 98%, 99% or higher identical) to SEQ ID NO: 12. In some embodiments, the anti-TCRβ V6 (e.g., anti-TCRβ V6-5*01) antibody molecule comprises a light chain (LC) variable region (VL) as set forth in SEQ ID NO: 17, or a sequence substantially identical (e.g., at least 80%, 85%, 90%, 92%, 95%, 97%, 98%, 99% or higher identical) to SEQ ID NO: 17. In some embodiments, the anti-TCRβ V6 (e.g., anti-TCRβ V6-5*01) antibody molecule comprises a light chain (LC) variable region (VL) as set forth in SEQ ID NO: 16, or a sequence substantially identical (e.g., at least 80%, 85%, 90%, 92%, 95%, 97%, 98%, 99% or higher identical) to SEQ ID NO: 16.

In some embodiments, the anti-TCRβ antibody comprises a sequence as described in Table 3, or a sequence substantially identical (e.g., at least 80%, 85%, 90%, 92%, 95%, 97%, 98%, 99% or higher identical) to a sequences sequence as described in Table 3.

In some embodiments, the anti-TCRβV antibody molecule comprises a heavy chain (HC) having a CDR1, a CDR2, and a CDR3; wherein the CDR1, CDR2 and CDR3 has a sequence as set forth in SEQ ID NO: 49, SEQ ID NO: 50, and SEQ ID NO: 51 respectively, according to Combined CDR1, CDR2, and CDR3 definition. In some embodiments, the anti-TCRβV antibody molecule comprises a heavy chain (HC) having a CDR1, a CDR2, and a CDR3; wherein the CDR1, CDR2 and CDR3 has a sequence as set forth in SEQ ID NO: 52, SEQ ID NO: 50, and SEQ ID NO: 51 respectively, according to Kabat CDR1, CDR2, and CDR3 definition. In some embodiments, the anti-TCRβV antibody molecule comprises a heavy chain (HC) having a CDR1, a CDR2, and a CDR3; wherein the CDR1, CDR2 and CDR3 has a sequence as set forth in SEQ ID NO: 53, SEQ ID NO: 54, and SEQ ID NO: 51 respectively, according to Chothia CDR1, CDR2, and CDR3 definition.

In some embodiments, the anti-TCRβV antibody molecule comprises a light chain (LC) having a CDR1, a CDR2, and a CDR3; wherein the CDR1, CDR2 and CDR3 has a sequence as set forth in SEQ ID NO: 55, SEQ ID NO: 56, and SEQ ID NO: 57 respectively, according to Combined CDR1, CDR2, and CDR3 definition. In some embodiments, the anti-TCRβV antibody molecule comprises a light chain (LC) having a CDR1, a CDR2, and a CDR3; wherein the CDR1, CDR2 and CDR3 has a sequence as set forth in SEQ ID NO: 55, SEQ ID NO: 56, and SEQ ID NO: 57 respectively according to Kabat CDR1, CDR2, and CDR3 definition. In some embodiments, the anti-TCRβV antibody molecule comprises a light chain (LC) having a CDR1, a CDR2, and a CDR3; wherein the CDR1, CDR2 and CDR3 has a sequence as set forth in SEQ ID NO: 55, SEQ ID NO: 56, and SEQ ID NO: 57 respectively, according to Chothia CDR1, CDR2, and CDR3 definition. In some embodiments, the anti-TCRβV antibody molecule comprises a heavy chain (HC) variable region (VH) as set forth in SEQ ID NO: 61, or a sequence substantially identical (e.g., at least 80%, 85%, 90%, 92%, 95%, 97%, 98%, 99% or higher identical) to SEQ ID NO: 61. In some embodiments, the anti-TCRβV antibody molecule comprises a light chain (LC) variable region (VL) as set forth in SEQ ID NO: 60, or a sequence substantially identical (e.g., at least 80%, 85%, 90%, 92%, 95%, 97%, 98%, 99% or higher identical) to SEQ ID NO: 60. In some embodiments, the anti-TCRβV antibody molecule comprises a heavy chain (HC) variable region (VH) as set forth in SEQ ID NO: 63, or a sequence substantially identical (e.g., at least 80%, 85%, 90%, 92%, 95%, 97%, 98%, 99% or higher identical) to SEQ ID NO: 64. In some embodiments, the anti-TCRβV antibody molecule comprises a heavy chain (HC) variable region (VL) as set forth in SEQ ID NO: 66, or a sequence substantially identical (e.g., at least 80%, 85%, 90%, 92%, 95%, 97%, 98%, 99% or higher identical) to SEQ ID NO: 66. In some embodiments, the anti-TCRβV antibody molecule comprises a heavy chain (HC) variable region (VL) as set forth in SEQ ID NO: 64, or a sequence substantially identical (e.g., at least 80%, 85%, 90%, 92%, 95%, 97%, 98%, 99% or higher identical) to SEQ ID NO: 64. In some embodiments, the anti-TCRβV antibody molecule comprises a heavy chain (HC) variable region (VL) as set forth in SEQ ID NO: 63, or a sequence substantially identical (e.g., at least 80%, 85%, 90%, 92%, 95%, 97%, 98%, 99% or higher identical) to SEQ ID NO: 63. In some embodiments, the anti-TCRβV antibody molecule comprises a light chain (LC) variable region (VL) as set forth in SEQ ID NO: 62, or a sequence substantially identical (e.g., at least 80%, 85%, 90%, 92%, 95%, 97%, 98%, 99% or higher identical) to SEQ ID NO: 62. In some embodiments, the anti-TCRβV antibody molecule comprises a light chain (LC) variable region (VL) as set forth in SEQ ID NO: 65, or a sequence substantially identical (e.g., at least 80%, 85%, 90%, 92%, 95%, 97%, 98%, 99% or higher identical) to SEQ ID NO: 65. In some embodiments, the anti-TCRβV antibody molecule comprises a light chain (LC) variable region (VL) as set forth in SEQ ID NO: 67, or a sequence substantially identical (e.g., at least 80%, 85%, 90%, 92%, 95%, 97%, 98%, 99% or higher identical) to SEQ ID NO: 67. In some embodiments, the anti-TCRβV antibody molecule comprises a light chain (LC) variable region (VL) as set forth in SEQ ID NO: 68, or a sequence substantially identical (e.g., at least 80%, 85%, 90%, 92%, 95%, 97%, 98%, 99% or higher identical) to SEQ ID NO: 68. In some embodiments, the anti-TCRβV antibody molecule comprises a light chain (LC) variable region (VL) as set forth in SEQ ID NO: 69, or a sequence substantially identical (e.g., at least 80%, 85%, 90%, 92%, 95%, 97%, 98%, 99% or higher identical) to SEQ ID NO: 69.

In some embodiments, the anti-TCRβ antibody comprises a sequence as described in Table 4, or a sequence substantially identical (e.g., at least 80%, 85%, 90%, 92%, 95%, 97%, 98%, 99% or higher identical) to a sequences sequence as described in Table 4.

In some embodiments, the anti-TCRβV antibody molecule comprises a heavy chain (HC) having a CDR1, a CDR2, and a CDR3; wherein the CDR1, CDR2 and CDR3 has a sequence as set forth in SEQ ID NO: 70, SEQ ID NO: 71, and SEQ ID NO: 72 respectively, according to Combined CDR1, CDR2, and CDR3 definition. In some embodiments, the anti-TCRβV antibody molecule comprises a heavy chain (HC) having a CDR1, a CDR2, and a CDR3; wherein the CDR1, CDR2 and CDR3 has a sequence as set forth in SEQ ID NO: 73, SEQ ID NO: 74, and SEQ ID NO: 72 respectively, according to Kabat CDR1, CDR2, and CDR3 definition. In some embodiments, the anti-TCRβV antibody molecule comprises a heavy chain (HC) having a CDR1, a CDR2, and a CDR3; wherein the CDR1, CDR2 and CDR3 has a sequence as set forth in SEQ ID NO: 75, SEQ ID NO: 71, and SEQ ID NO: 72 respectively, according to Chothia CDR1, CDR2, and CDR3 definition.

In some embodiments, the anti-TCRβV antibody molecule comprises a light chain (LC) having a CDR1, a CDR2, and a CDR3; wherein the CDR1, CDR2 and CDR3 has a sequence as set forth in SEQ ID NO: 76, SEQ ID NO: 77, and SEQ ID NO: 78 respectively, according to Combined CDR1, CDR2, and CDR3 definition. In some embodiments, the anti-TCRβV antibody molecule comprises a light chain (LC) having a CDR1, a CDR2, and a CDR3; wherein the CDR1, CDR2 and CDR3 has a sequence as set forth in SEQ ID NO: 76, SEQ ID NO: 77, and SEQ ID NO: 78 respectively according to Kabat CDR1, CDR2, and CDR3 definition. In some embodiments, the anti-TCRβV antibody molecule comprises a light chain (LC) having a CDR1, a CDR2, and a CDR3; wherein the CDR1, CDR2 and CDR3 has a sequence as set forth in SEQ ID NO: 76, SEQ ID NO: 77, and SEQ ID NO: 78 respectively, according to Chothia CDR1, CDR2, and CDR3 definition. In some embodiments, the anti-TCRβV antibody molecule comprises a heavy chain (HC) variable region (VH) as set forth in SEQ ID NO: 82, or a sequence substantially identical (e.g., at least 80%, 85%, 90%, 92%, 95%, 97%, 98%, 99% or higher identical) to SEQ ID NO: 82. In some embodiments, the anti-TCRβV antibody molecule comprises a light chain (LC) variable region (VL) as set forth in SEQ ID NO: 81, or a sequence substantially identical (e.g., at least 80%, 85%, 90%, 92%, 95%, 97%, 98%, 99% or higher identical) to SEQ ID NO: 81. In some embodiments, the anti-TCRβV antibody molecule comprises a heavy chain (HC) variable region (VH) as set forth in SEQ ID NO: 83, or a sequence substantially identical (e.g., at least 80%, 85%, 90%, 92%, 95%, 97%, 98%, 99% or higher identical) to SEQ ID NO: 83. In some embodiments, the anti-TCRβV antibody molecule comprises a heavy chain (HC) variable region (VH) as set forth in SEQ ID NO: 84, or a sequence substantially identical (e.g., at least 80%, 85%, 90%, 92%, 95%, 97%, 98%, 99% or higher identical) to SEQ ID NO: 84. In some embodiments, the anti-TCRβV antibody molecule comprises a heavy chain (HC) variable region (VL) as set forth in SEQ ID NO: 85, or a sequence substantially identical (e.g., at least 80%, 85%, 90%, 92%, 95%, 97%, 98%, 99% or higher identical) to SEQ ID NO: 85. In some embodiments, the anti-TCRβV antibody molecule comprises a heavy chain (HC) variable region (VH) as set forth in SEQ ID NO: 86, or a sequence substantially identical (e.g., at least 80%, 85%, 90%, 92%, 95%, 97%, 98%, 99% or higher identical) to SEQ ID NO: 86. In some embodiments, the anti-TCRβV antibody molecule comprises a heavy chain (HC) variable region (VH) as set forth in SEQ ID NO: 87, or a sequence substantially identical (e.g., at least 80%, 85%, 90%, 92%, 95%, 97%, 98%, 99% or higher identical) to SEQ ID NO: 87. In some embodiments, the anti-TCRβV antibody molecule comprises a light chain (LC) variable region (VL) as set forth in SEQ ID NO: 88, or a sequence substantially identical (e.g., at least 80%, 85%, 90%, 92%, 95%, 97%, 98%, 99% or higher identical) to SEQ ID NO: 88. In some embodiments, the anti-TCRβV antibody molecule comprises a light chain (HC) variable region (VL) as set forth in SEQ ID NO: 89, or a sequence substantially identical (e.g., at least 80%, 85%, 90%, 92%, 95%, 97%, 98%, 99% or higher identical) to SEQ ID NO: 89. In some embodiments, the anti-TCRβV antibody molecule comprises a light chain (LC) variable region (VL) as set forth in SEQ ID NO: 90, or a sequence substantially identical (e.g., at least 80%, 85%, 90%, 92%, 95%, 97%, 98%, 99% or higher identical) to SEQ ID NO: 90. In some embodiments, the anti-TCRβV antibody molecule comprises a light chain (LC) variable region (VL) as set forth in SEQ ID NO: 91, or a sequence substantially identical (e.g., at least 80%, 85%, 90%, 92%, 95%, 97%, 98%, 99% or higher identical) to SEQ ID NO: 91. In some embodiments, the anti-TCRβV antibody molecule comprises a light chain (LC) variable region (VL) as set forth in SEQ ID NO: 92, or a sequence substantially identical (e.g., at least 80%, 85%, 90%, 92%, 95%, 97%, 98%, 99% or higher identical) to SEQ ID NO: 92. In some embodiments, the anti-TCRβV antibody molecule comprises a light chain (LC) variable region (VL) as set forth in SEQ ID NO: 93, or a sequence substantially identical (e.g., at least 80%, 85%, 90%, 92%, 95%, 97%, 98%, 99% or higher identical) to SEQ ID NO: 93. In some embodiments, the anti-TCRβV antibody molecule comprises a light chain (LC) variable region (VL) as set forth in SEQ ID NO: 94, or a sequence substantially identical (e.g., at least 80%, 85%, 90%, 92%, 95%, 97%, 98%, 99% or higher identical) to SEQ ID NO: 94. In some embodiments, the anti-TCRβV antibody molecule comprises a light chain (LC) variable region (VL) as set forth in SEQ ID NO: 95, or a sequence substantially identical (e.g., at least 80%, 85%, 90%, 92%, 95%, 97%, 98%, 99% or higher identical) to SEQ ID NO: 95.

In some embodiments, the anti-TCRβ antibody comprises a sequence as described in Table 5, or a sequence substantially identical (e.g., at least 80%, 85%, 90%, 92%, 95%, 97%, 98%, 99% or higher identical) to a sequences sequence as described in Table 5.

In some embodiments, the anti-TCRβV antibody molecule comprises a heavy chain (HC) variable region (VH) as set forth in SEQ ID NO: 108, or a sequence substantially identical (e.g., at least 80%, 85%, 90%, 92%, 95%, 97%, 98%, 99% or higher identical) to SEQ ID NO: 108. In some embodiments, the anti-TCRβV antibody molecule comprises a heavy chain (HC) variable region (VH) as set forth in SEQ ID NO: 109, or a sequence substantially identical (e.g., at least 80%, 85%, 90%, 92%, 95%, 97%, 98%, 99% or higher identical) to SEQ ID NO: 109. In some embodiments, the anti-TCRβV antibody molecule comprises a heavy chain (HC) variable region (VH) as set forth in SEQ ID NO: 110, or a sequence substantially identical (e.g., at least 80%, 85%, 90%, 92%, 95%, 97%, 98%, 99% or higher identical) to SEQ ID NO: 110. In some embodiments, the anti-TCRβV antibody molecule comprises a heavy chain (HC) variable region (VH) as set forth in SEQ ID NO: 111, or a sequence substantially identical (e.g., at least 80%, 85%, 90%, 92%, 95%, 97%, 98%, 99% or higher identical) to SEQ ID NO: 111. In some embodiments, the anti-TCRβV antibody molecule comprises a light chain (LC) variable region (VL) as set forth in SEQ ID NO: 112, or a sequence substantially identical (e.g., at least 80%, 85%, 90%, 92%, 95%, 97%, 98%, 99% or higher identical) to SEQ ID NO: 112. In some embodiments, the anti-TCRβV antibody molecule comprises a light chain (LC) variable region (VL) as set forth in SEQ ID NO: 113, or a sequence substantially identical (e.g., at least 80%, 85%, 90%, 92%, 95%, 97%, 98%, 99% or higher identical) to SEQ ID NO: 113. In some embodiments, the anti-TCRβV antibody molecule comprises a light chain (LC) variable region (VL) as set forth in SEQ ID NO: 114, or a sequence substantially identical (e.g., at least 80%, 85%, 90%, 92%, 95%, 97%, 98%, 99% or higher identical) to SEQ ID NO: 114.

In some embodiments, the anti-TCRβV antibody molecule comprises a heavy chain (HC) variable region (VH) as set forth in SEQ ID NO: 127, or a sequence substantially identical (e.g., at least 80%, 85%, 90%, 92%, 95%, 97%, 98%, 99% or higher identical) to SEQ ID NO: 127. In some embodiments, the anti-TCRβV antibody molecule comprises a heavy chain (HC) variable region (VH) as set forth in SEQ ID NO: 128, or a sequence substantially identical (e.g., at least 80%, 85%, 90%, 92%, 95%, 97%, 98%, 99% or higher identical) to SEQ ID NO: 128. In some embodiments, the anti-TCRβV antibody molecule comprises a heavy chain (HC) variable region (VH) as set forth in SEQ ID NO: 129, or a sequence substantially identical (e.g., at least 80%, 85%, 90%, 92%, 95%, 97%, 98%, 99% or higher identical) to SEQ ID NO: 129. In some embodiments, the anti-TCRβV antibody molecule comprises a heavy chain (HC) variable region (VH) as set forth in SEQ ID NO: 130, or a sequence substantially identical (e.g., at least 80%, 85%, 90%, 92%, 95%, 97%, 98%, 99% or higher identical) to SEQ ID NO: 130. In some embodiments, the anti-TCRβV antibody molecule comprises a heavy chain (HC) variable region (VH) as set forth in SEQ ID NO: 131, or a sequence substantially identical (e.g., at least 80%, 85%, 90%, 92%, 95%, 97%, 98%, 99% or higher identical) to SEQ ID NO: 131.

In some embodiments, the anti-TCRβV antibody molecule comprises a light chain (LC) variable region (VL) as set forth in SEQ ID NO: 132, or a sequence substantially identical (e.g., at least 80%, 85%, 90%, 92%, 95%, 97%, 98%, 99% or higher identical) to SEQ ID NO: 132. In some embodiments, the anti-TCRβV antibody molecule comprises a light chain (LC) variable region (VL) as set forth in SEQ ID NO: 133, or a sequence substantially identical (e.g., at least 80%, 85%, 90%, 92%, 95%, 97%, 98%, 99% or higher identical) to SEQ ID NO: 133. In some embodiments, the anti-TCRβV antibody molecule comprises a light chain (LC) variable region (VL) as set forth in SEQ ID NO: 134, or a sequence substantially identical (e.g., at least 80%, 85%, 90%, 92%, 95%, 97%, 98%, 99% or higher identical) to SEQ ID NO: 134. In some embodiments, the anti-TCRβV antibody molecule comprises a light chain (LC) variable region (VL) as set forth in SEQ ID NO: 135, or a sequence substantially identical (e.g., at least 80%, 85%, 90%, 92%, 95%, 97%, 98%, 99% or higher identical) to SEQ ID NO: 135. In some embodiments, the anti-TCRβV antibody molecule comprises a light chain (LC) variable region (VL) as set forth in SEQ ID NO: 136, or a sequence substantially identical (e.g., at least 80%, 85%, 90%, 92%, 95%, 97%, 98%, 99% or higher identical) to SEQ ID NO: 136.

In some embodiments, the anti-TCRβV antibody molecule comprises a heavy chain (HC) variable region (VH) as set forth in SEQ ID NO: 149, or a sequence substantially identical (e.g., at least 80%, 85%, 90%, 92%, 95%, 97%, 98%, 99% or higher identical) to SEQ ID NO: 149. In some embodiments, the anti-TCRβV antibody molecule comprises a heavy chain (HC) variable region (VH) as set forth in SEQ ID NO: 150, or a sequence substantially identical (e.g., at least 80%, 85%, 90%, 92%, 95%, 97%, 98%, 99% or higher identical) to SEQ ID NO: 150. In some embodiments, the anti-TCRβV antibody molecule comprises a heavy chain (HC) variable region (VH) as set forth in SEQ ID NO: 151, or a sequence substantially identical (e.g., at least 80%, 85%, 90%, 92%, 95%, 97%, 98%, 99% or higher identical) to SEQ ID NO: 151. In some embodiments, the anti-TCRβV antibody molecule comprises a heavy chain (HC) variable region (VH) as set forth in SEQ ID NO: 152, or a sequence substantially identical (e.g., at least 80%, 85%, 90%, 92%, 95%, 97%, 98%, 99% or higher identical) to SEQ ID NO: 152. In some embodiments, the anti-TCRβV antibody molecule comprises a heavy chain (HC) variable region (VH) as set forth in SEQ ID NO: 153, or a sequence substantially identical (e.g., at least 80%, 85%, 90%, 92%, 95%, 97%, 98%, 99% or higher identical) to SEQ ID NO: 153.

In some embodiments, the anti-TCRβV antibody molecule comprises a light chain (LC) variable region (VL) as set forth in SEQ ID NO: 154, or a sequence substantially identical (e.g., at least 80%, 85%, 90%, 92%, 95%, 97%, 98%, 99% or higher identical) to SEQ ID NO: 154. In some embodiments, the anti-TCRβV antibody molecule comprises a light chain (LC) variable region (VL) as set forth in SEQ ID NO: 155, or a sequence substantially identical (e.g., at least 80%, 85%, 90%, 92%, 95%, 97%, 98%, 99% or higher identical) to SEQ ID NO: 155. In some embodiments, the anti-TCRβV antibody molecule comprises a light chain (LC) variable region (VL) as set forth in SEQ ID NO: 156, or a sequence substantially identical (e.g., at least 80%, 85%, 90%, 92%, 95%, 97%, 98%, 99% or higher identical) to SEQ ID NO: 156. In some embodiments, the anti-TCRβV antibody molecule comprises a light chain (LC) variable region (VL) as set forth in SEQ ID NO: 157, or a sequence substantially identical (e.g., at least 80%, 85%, 90%, 92%, 95%, 97%, 98%, 99% or higher identical) to SEQ ID NO: 157. In some embodiments, the anti-TCRβV antibody molecule comprises a light chain (LC) variable region (VL) as set forth in SEQ ID NO: 158, or a sequence substantially identical (e.g., at least 80%, 85%, 90%, 92%, 95%, 97%, 98%, 99% or higher identical) to SEQ ID NO: 158.

In some embodiments, the anti-TCRβV antibody molecule comprises a heavy chain (HC) variable region (VH) as set forth in SEQ ID NO: 170, or a sequence substantially identical (e.g., at least 80%, 85%, 90%, 92%, 95%, 97%, 98%, 99% or higher identical) to SEQ ID NO: 170. In some embodiments, the anti-TCRβV antibody molecule comprises a heavy chain (HC) variable region (VH) as set forth in SEQ ID NO: 171, or a sequence substantially identical (e.g., at least 80%, 85%, 90%, 92%, 95%, 97%, 98%, 99% or higher identical) to SEQ ID NO: 171. In some embodiments, the anti-TCRβV antibody molecule comprises a heavy chain (HC) variable region (VH) as set forth in SEQ ID NO: 172, or a sequence substantially identical (e.g., at least 80%, 85%, 90%, 92%, 95%, 97%, 98%, 99% or higher identical) to SEQ ID NO: 172. In some embodiments, the anti-TCRβV antibody molecule comprises a heavy chain (HC) variable region (VH) as set forth in SEQ ID NO: 173, or a sequence substantially identical (e.g., at least 80%, 85%, 90%, 92%, 95%, 97%, 98%, 99% or higher identical) to SEQ ID NO: 173. In some embodiments, the anti-TCRβV antibody molecule comprises a heavy chain (HC) variable region (VH) as set forth in SEQ ID NO: 174, or a sequence substantially identical (e.g., at least 80%, 85%, 90%, 92%, 95%, 97%, 98%, 99% or higher identical) to SEQ ID NO: 174.

In some embodiments, the anti-TCRβV antibody molecule comprises a light chain (LC) variable region (VL) as set forth in SEQ ID NO: 175, or a sequence substantially identical (e.g., at least 80%, 85%, 90%, 92%, 95%, 97%, 98%, 99% or higher identical) to SEQ ID NO: 175. In some embodiments, the anti-TCRβV antibody molecule comprises a light chain (LC) variable region (VL) as set forth in SEQ ID NO: 176, or a sequence substantially identical (e.g., at least 80%, 85%, 90%, 92%, 95%, 97%, 98%, 99% or higher identical) to SEQ ID NO: 176. In some embodiments, the anti-TCRβV antibody molecule comprises a light chain (LC) variable region (VL) as set forth in SEQ ID NO: 177, or a sequence substantially identical (e.g., at least 80%, 85%, 90%, 92%, 95%, 97%, 98%, 99% or higher identical) to SEQ ID NO: 177. In some embodiments, the anti-TCRβV antibody molecule comprises a light chain (LC) variable region (VL) as set forth in SEQ ID NO: 178, or a sequence substantially identical (e.g., at least 80%, 85%, 90%, 92%, 95%, 97%, 98%, 99% or higher identical) to SEQ ID NO: 178. In some embodiments, the anti-TCRβV antibody molecule comprises a light chain (LC) variable region (VL) as set forth in SEQ ID NO: 179, or a sequence substantially identical (e.g., at least 80%, 85%, 90%, 92%, 95%, 97%, 98%, 99% or higher identical) to SEQ ID NO: 179. In some embodiments, the anti-TCRβV antibody molecule comprises a light chain (LC) variable region (VL) as set forth in SEQ ID NO: 180, or a sequence substantially identical (e.g., at least 80%, 85%, 90%, 92%, 95%, 97%, 98%, 99% or higher identical) to SEQ ID NO: 180. In some embodiments, the anti-TCRβV antibody molecule comprises a light chain (LC) variable region (VL) as set forth in SEQ ID NO: 181, or a sequence substantially identical (e.g., at least 80%, 85%, 90%, 92%, 95%, 97%, 98%, 99% or higher identical) to SEQ ID NO: 181.

In some embodiments, the anti-TCRβV antibody molecule comprises a heavy chain (HC) variable region (VH) as set forth in SEQ ID NO: 194, or a sequence substantially identical (e.g., at least 80%, 85%, 90%, 92%, 95%, 97%, 98%, 99% or higher identical) to SEQ ID NO: 194. In some embodiments, the anti-TCRβV antibody molecule comprises a heavy chain (HC) variable region (VH) as set forth in SEQ ID NO: 195, or a sequence substantially identical (e.g., at least 80%, 85%, 90%, 92%, 95%, 97%, 98%, 99% or higher identical) to SEQ ID NO: 195. In some embodiments, the anti-TCRβV antibody molecule comprises a heavy chain (HC) variable region (VH) as set forth in SEQ ID NO: 196, or a sequence substantially identical (e.g., at least 80%, 85%, 90%, 92%, 95%, 97%, 98%, 99% or higher identical) to SEQ ID NO: 196. In some embodiments, the anti-TCRβV antibody molecule comprises a heavy chain (HC) variable region (VH) as set forth in SEQ ID NO: 197, or a sequence substantially identical (e.g., at least 80%, 85%, 90%, 92%, 95%, 97%, 98%, 99% or higher identical) to SEQ ID NO: 197. In some embodiments, the anti-TCRβV antibody molecule comprises a heavy chain (HC) variable region (VH) as set forth in SEQ ID NO: 198, or a sequence substantially identical (e.g., at least 80%, 85%, 90%, 92%, 95%, 97%, 98%, 99% or higher identical) to SEQ ID NO: 198. In some embodiments, the anti-TCRβV antibody molecule comprises a heavy chain (HC) variable region (VH) as set forth in SEQ ID NO: 199, or a sequence substantially identical (e.g., at least 80%, 85%, 90%, 92%, 95%, 97%, 98%, 99% or higher identical) to SEQ ID NO: 199.

In some embodiments, the anti-TCRβV antibody molecule comprises a light chain (LC) variable region (VL) as set forth in SEQ ID NO: 200, or a sequence substantially identical (e.g., at least 80%, 85%, 90%, 92%, 95%, 97%, 98%, 99% or higher identical) to SEQ ID NO: 200. In some embodiments, the anti-TCRβV antibody molecule comprises a light chain (LC) variable region (VL) as set forth in SEQ ID NO: 201, or a sequence substantially identical (e.g., at least 80%, 85%, 90%, 92%, 95%, 97%, 98%, 99% or higher identical) to SEQ ID NO: 201. In some embodiments, the anti-TCRβV antibody molecule comprises a light chain (LC) variable region (VL) as set forth in SEQ ID NO: 202, or a sequence substantially identical (e.g., at least 80%, 85%, 90%, 92%, 95%, 97%, 98%, 99% or higher identical) to SEQ ID NO: 202. In some embodiments, the anti-TCRβV antibody molecule comprises a light chain (LC) variable region (VL) as set forth in SEQ ID NO: 203, or a sequence substantially identical (e.g., at least 80%, 85%, 90%, 92%, 95%, 97%, 98%, 99% or higher identical) to SEQ ID NO: 203. In some embodiments, the anti-TCRβV antibody molecule comprises a light chain (LC) variable region (VL) as set forth in SEQ ID NO: 204, or a sequence substantially identical (e.g., at least 80%, 85%, 90%, 92%, 95%, 97%, 98%, 99% or higher identical) to SEQ ID NO: 204. In some embodiments, the anti-TCRβV antibody molecule comprises a light chain (LC) variable region (VL) as set forth in SEQ ID NO: 205, or a sequence substantially identical (e.g., at least 80%, 85%, 90%, 92%, 95%, 97%, 98%, 99% or higher identical) to SEQ ID NO: 205.

In some embodiments, the anti-TCRβV antibody molecule comprises a heavy chain (HC) variable region (VH) as set forth in SEQ ID NO: 217, or a sequence substantially identical (e.g., at least 80%, 85%, 90%, 92%, 95%, 97%, 98%, 99% or higher identical) to SEQ ID NO: 217. In some embodiments, the anti-TCRβV antibody molecule comprises a heavy chain (HC) variable region (VH) as set forth in SEQ ID NO: 218, or a sequence substantially identical (e.g., at least 80%, 85%, 90%, 92%, 95%, 97%, 98%, 99% or higher identical) to SEQ ID NO: 218. In some embodiments, the anti-TCRβV antibody molecule comprises a heavy chain (HC) variable region (VH) as set forth in SEQ ID NO: 219, or a sequence substantially identical (e.g., at least 80%, 85%, 90%, 92%, 95%, 97%, 98%, 99% or higher identical) to SEQ ID NO: 219. In some embodiments, the anti-TCRβV antibody molecule comprises a heavy chain (HC) variable region (VH) as set forth in SEQ ID NO: 220, or a sequence substantially identical (e.g., at least 80%, 85%, 90%, 92%, 95%, 97%, 98%, 99% or higher identical) to SEQ ID NO: 220. In some embodiments, the anti-TCRβV antibody molecule comprises a heavy chain (HC) variable region (VH) as set forth in SEQ ID NO: 221, or a sequence substantially identical (e.g., at least 80%, 85%, 90%, 92%, 95%, 97%, 98%, 99% or higher identical) to SEQ ID NO: 221.

In some embodiments, the anti-TCRβV antibody molecule comprises a light chain (LC) variable region (VL) as set forth in SEQ ID NO: 222, or a sequence substantially identical (e.g., at least 80%, 85%, 90%, 92%, 95%, 97%, 98%, 99% or higher identical) to SEQ ID NO: 222. In some embodiments, the anti-TCRβV antibody molecule comprises a light chain (LC) variable region (VL) as set forth in SEQ ID NO: 223, or a sequence substantially identical (e.g., at least 80%, 85%, 90%, 92%, 95%, 97%, 98%, 99% or higher identical) to SEQ ID NO: 223. In some embodiments, the anti-TCRβV antibody molecule comprises a light chain (LC) variable region (VL) as set forth in SEQ ID NO: 224, or a sequence substantially identical (e.g., at least 80%, 85%, 90%, 92%, 95%, 97%, 98%, 99% or higher identical) to SEQ ID NO: 224. In some embodiments, the anti-TCRβV antibody molecule comprises a light chain (LC) variable region (VL) as set forth in SEQ ID NO: 225, or a sequence substantially identical (e.g., at least 80%, 85%, 90%, 92%, 95%, 97%, 98%, 99% or higher identical) to SEQ ID NO: 225. In some embodiments, the anti-TCRβV antibody molecule comprises a light chain (LC) variable region (VL) as set forth in SEQ ID NO: 226, or a sequence substantially identical (e.g., at least 80%, 85%, 90%, 92%, 95%, 97%, 98%, 99% or higher identical) to SEQ ID NO: 226. In some embodiments, the anti-TCRβV antibody molecule comprises a light chain (LC) variable region (VL) as set forth in SEQ ID NO: 227, or a sequence substantially identical (e.g., at least 80%, 85%, 90%, 92%, 95%, 97%, 98%, 99% or higher identical) to SEQ ID NO: 227.

In some embodiments, the anti-TCRβV antibody molecule comprises a heavy chain (HC) variable region (VH) as set forth in SEQ ID NO: 262, or a sequence substantially identical (e.g., at least 80%, 85%, 90%, 92%, 95%, 97%, 98%, 99% or higher identical) to SEQ ID NO: 262. In some embodiments, the anti-TCRβV antibody molecule comprises a heavy chain (HC) variable region (VH) as set forth in SEQ ID NO: 263, or a sequence substantially identical (e.g., at least 80%, 85%, 90%, 92%, 95%, 97%, 98%, 99% or higher identical) to SEQ ID NO: 263. In some embodiments, the anti-TCRβV antibody molecule comprises a heavy chain (HC) variable region (VH) as set forth in SEQ ID NO: 264, or a sequence substantially identical (e.g., at least 80%, 85%, 90%, 92%, 95%, 97%, 98%, 99% or higher identical) to SEQ ID NO: 264. In some embodiments, the anti-TCRβV antibody molecule comprises a heavy chain (HC) variable region (VH) as set forth in SEQ ID NO: 310, or a sequence substantially identical (e.g., at least 80%, 85%, 90%, 92%, 95%, 97%, 98%, 99% or higher identical) to SEQ ID NO: 265. In some embodiments, the anti-TCRβV antibody molecule comprises a heavy chain (HC) variable region (VH) as set forth in SEQ ID NO: 311, or a sequence substantially identical (e.g., at least 80%, 85%, 90%, 92%, 95%, 97%, 98%, 99% or higher identical) to SEQ ID NO: 265.

In some embodiments, the anti-TCRβV antibody molecule comprises a light chain (LC) variable region (VL) as set forth in SEQ ID NO: 266, or a sequence substantially identical (e.g., at least 80%, 85%, 90%, 92%, 95%, 97%, 98%, 99% or higher identical) to SEQ ID NO: 266. In some embodiments, the anti-TCRβV antibody molecule comprises a light chain (LC) variable region (VL) as set forth in SEQ ID NO: 267, or a sequence substantially identical (e.g., at least 80%, 85%, 90%, 92%, 95%, 97%, 98%, 99% or higher identical) to SEQ ID NO: 267. In some embodiments, the anti-TCRβV antibody molecule comprises a light chain (LC) variable region (VL) as set forth in SEQ ID NO: 268, or a sequence substantially identical (e.g., at least 80%, 85%, 90%, 92%, 95%, 97%, 98%, 99% or higher identical) to SEQ ID NO: 268. In some embodiments, the anti-TCRβV antibody molecule comprises a light chain (LC) variable region (VL) as set forth in SEQ ID NO: 269, or a sequence substantially identical (e.g., at least 80%, 85%, 90%, 92%, 95%, 97%, 98%, 99% or higher identical) to SEQ ID NO: 269.

In some embodiments, the anti-TCRβV antibody molecule comprises a heavy chain (HC) variable region (VH) as set forth in SEQ ID NO: 240, or a sequence substantially identical (e.g., at least 80%, 85%, 90%, 92%, 95%, 97%, 98%, 99% or higher identical) to SEQ ID NO: 240. In some embodiments, the anti-TCRβV antibody molecule comprises a heavy chain (HC) variable region (VH) as set forth in SEQ ID NO: 241, or a sequence substantially identical (e.g., at least 80%, 85%, 90%, 92%, 95%, 97%, 98%, 99% or higher identical) to SEQ ID NO: 241. In some embodiments, the anti-TCRβV antibody molecule comprises a heavy chain (HC) variable region (VH) as set forth in SEQ ID NO: 242, or a sequence substantially identical (e.g., at least 80%, 85%, 90%, 92%, 95%, 97%, 98%, 99% or higher identical) to SEQ ID NO: 242. In some embodiments, the anti-TCRβV antibody molecule comprises a heavy chain (HC) variable region (VH) as set forth in SEQ ID NO: 243, or a sequence substantially identical (e.g., at least 80%, 85%, 90%, 92%, 95%, 97%, 98%, 99% or higher identical) to SEQ ID NO: 243.

In some embodiments, the anti-TCRβV antibody molecule comprises a light chain (LC) variable region (VL) as set forth in SEQ ID NO: 244, or a sequence substantially identical (e.g., at least 80%, 85%, 90%, 92%, 95%, 97%, 98%, 99% or higher identical) to SEQ ID NO: 244. In some embodiments, the anti-TCRβV antibody molecule comprises a light chain (LC) variable region (VL) as set forth in SEQ ID NO: 245, or a sequence substantially identical (e.g., at least 80%, 85%, 90%, 92%, 95%, 97%, 98%, 99% or higher identical) to SEQ ID NO: 245. In some embodiments, the anti-TCRβV antibody molecule comprises a light chain (LC) variable region (VL) as set forth in SEQ ID NO: 246, or a sequence substantially identical (e.g., at least 80%, 85%, 90%, 92%, 95%, 97%, 98%, 99% or higher identical) to SEQ ID NO: 246. In some embodiments, the anti-TCRβV antibody molecule comprises a light chain (LC) variable region (VL) as set forth in SEQ ID NO: 247, or a sequence substantially identical (e.g., at least 80%, 85%, 90%, 92%, 95%, 97%, 98%, 99% or higher identical) to SEQ ID NO: 247. In some embodiments, the anti-TCRβV antibody molecule comprises a light chain (LC) variable region (VL) as set forth in SEQ ID NO: 248, or a sequence substantially identical (e.g., at least 80%, 85%, 90%, 92%, 95%, 97%, 98%, 99% or higher identical) to SEQ ID NO: 248. In some embodiments, the anti-TCRβV antibody molecule comprises a light chain (LC) variable region (VL) as set forth in SEQ ID NO: 249, or a sequence substantially identical (e.g., at least 80%, 85%, 90%, 92%, 95%, 97%, 98%, 99% or higher identical) to SEQ ID NO: 249.

In some embodiments, the anti-TCRβV antibody molecule comprises a heavy chain (HC) variable region (VH) as set forth in SEQ ID NO: 282, or a sequence substantially identical (e.g., at least 80%, 85%, 90%, 92%, 95%, 97%, 98%, 99% or higher identical) to SEQ ID NO: 282. In some embodiments, the anti-TCRβV antibody molecule comprises a heavy chain (HC) variable region (VH) as set forth in SEQ ID NO: 283, or a sequence substantially identical (e.g., at least 80%, 85%, 90%, 92%, 95%, 97%, 98%, 99% or higher identical) to SEQ ID NO: 283. In some embodiments, the anti-TCRβV antibody molecule comprises a heavy chain (HC) variable region (VH) as set forth in SEQ ID NO: 284, or a sequence substantially identical (e.g., at least 80%, 85%, 90%, 92%, 95%, 97%, 98%, 99% or higher identical) to SEQ ID NO: 284.

In some embodiments, the anti-TCRβV antibody molecule comprises a light chain (LC) variable region (VL) as set forth in SEQ ID NO: 285, or a sequence substantially identical (e.g., at least 80%, 85%, 90%, 92%, 95%, 97%, 98%, 99% or higher identical) to SEQ ID NO: 285. In some embodiments, the anti-TCRβV antibody molecule comprises a light chain (LC) variable region (VL) as set forth in SEQ ID NO: 286, or a sequence substantially identical (e.g., at least 80%, 85%, 90%, 92%, 95%, 97%, 98%, 99% or higher identical) to SEQ ID NO: 286. In some embodiments, the anti-TCRβV antibody molecule comprises a light chain (LC) variable region (VL) as set forth in SEQ ID NO: 287, or a sequence substantially identical (e.g., at least 80%, 85%, 90%, 92%, 95%, 97%, 98%, 99% or higher identical) to SEQ ID NO: 287. In some embodiments, the anti-TCRβV antibody molecule comprises a light chain (LC) variable region (VL) as set forth in SEQ ID NO: 288, or a sequence substantially identical (e.g., at least 80%, 85%, 90%, 92%, 95%, 97%, 98%, 99% or higher identical) to SEQ ID NO: 288.

In some embodiments, the first agent, upon binding to the TCRβV region, results in expansion of T cells ex vivo. In some embodiments, binding of the first agent to the TCRβV region results in an increase of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of IL-2 as measured by an assay described herein.

In some embodiments, the methods described herein result in expansion of T cells ex vivo with less or no production of cytokines associated with CRS, e.g., IL-6, IL-1beta and TNF alpha; and enhanced and/or delayed production of IL-2 and IFNγ. In some embodiments, the first agent, upon binding to the TCRβV region, results in one, two, three, four, five, six, seven, eight, nine, ten or more (e.g., all) of the following: (i) reduced level, e.g., expression level, and/or activity of IL-1β; (ii) reduced level, e.g., expression level, and/or activity of IL-6; (iii) reduced level, e.g., expression level, and/or activity of TNFα; (iv) increased level, e.g., expression level, and/or activity of IL-2; (v) a delay, e.g., at least 1, 2, 3, 4, 5, 6, 7, 8, 9, 10 or more hours delay, in increased level, e.g., expression level, and/or activity of IL-2; (vi) a delay, e.g., at least 1, 2, 3, 4, 5, 6, 7, 8, 9, 10 hours delay, in increased level, e.g., expression level, and/or activity of IFNγ; (vii) reduced T cell proliferation kinetics; or (viii) reduced cytokine storm, e.g., cytokine release syndrome (CRS), e.g., as measured by an assay described herein; (ix) cell killing, e.g., target cell killing, e.g. cancer cell killing, e.g., as measured by an assay described herein; (x) increased level, e.g., expression level, and/or activity of IL-15; or (xi) increased Natural Killer (NK) cell proliferation, e.g., expansion, compared to an antibody that binds to: a CD3 molecule, e.g., CD3 epsilon (CD3e) molecule; or a TCR alpha (TCRα) molecule.

In some embodiments, the methods of expanding T cells ex vivo described herein result in expansion of a subset of memory effector T cells, e.g., T effector memory (TEM) cells, e.g., TEM cells expressing CD45RA (TEMRA) cells. In some embodiments, the first agent, upon binding to the TCRβV region, results in expansion, e.g., at least about 1.1-10 fold expansion (e.g., at least about 1.1, 1.2, 1.3, 1.4, 1.5, 2, 3, 4, 5, 6, 7, 8, 9, or 10 fold expansion), of a population of memory T cells, e.g., TEMRA cells. In some embodiments, the population of expanded T effector memory cells comprises cells which: (i) have a detectable level of CD45RA, e.g., express or re-express CD45RA; (ii) have low or no expression of CCR7; and/or (iii) have a detectable level of CD95, e.g., express CD95, e.g., a population of CD45RA+, CCR7−, CD95+ T cells, optionally wherein the T cells comprise CD3+, CD4+ or CD8+ T cells. In some embodiments, binding of the first agent to the TCRβV region results in a reduction of at least 2, 5, 10, 20, 50, 100, or 200 fold, or at least 2-200 fold (e.g., 5-150, 10-100, 20-50 fold) in the expression level and or activity of IL-1β compared to a population of memory T cells that are expanded in absence of the first agent, as measured by an assay described herein.

In some embodiments, binding of the first agent to the TCRβV region results in a reduction of at least 2, 5, 10, 20, 50, 100, 200, 300, 400, 500, 600, 700, 800, 900, or 1000 fold, or at least 2-1000 fold (e.g., 5-900, 10-800, 20-700, 50-600, 100-500, or 200-400 fold) in the expression level and or activity of IL-6 compared to a population of memory T cells that are expanded in absence of the first agent, as measured with respect to by an assay described herein.

In some embodiments, binding of the first agent to the TCRβV region results in a reduction of at least 2, 5, 10, 20, 50, 100, 200, 300, 400, 500, 600, 700, 800, 900, 1000, or 2000 fold, or at least 2-2000 fold (e.g., 5-1000, 10-900, 20-800, 50-700, 100-600, 200-500, or 300-400 fold) in the expression level and or activity of TNFα compared to a population of memory T cells that are expanded in absence of the first agent, as measured by an assay described herein.

In some embodiments, T cells are activated and expanded and expanded ex vivo using an anti-TCRβV antibody described herein. In some embodiments, the TCRβV antibody comprises a humanized antibody CDR or variable region as listed in Tables 2, 3, 4, or 5, or a sequence substantially identical (e.g., at least 80%, 85%, 90%, 92%, 95%, 97%, 98%, 99% or higher identical) to any of the aforesaid sequences. In some embodiments, the anti-TCRβV antibody molecule comprises a heavy chain constant region for an IgG4, e.g., a human IgG4. In still another embodiment, the anti-TCRβV antibody molecule includes a heavy chain constant region for an IgG1, e.g., a human IgG1. In some embodiments, the anti-TCRβV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1. In one embodiment, the heavy chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule or parts thereof may be a humanized version selected from an antibody designated as BHM1709, H131, H131-3, TM29, 16G8, TM23, MPB2D5, CAS1.1.3, IMMU222, REA1062, JOVI-3, 5511, MH3-2, and 4H11.

In some embodiments, the anti-TCRβV antibody molecule is a full antibody or fragment thereof (e.g., a Fab, F(ab′)₂, Fv, or a single chain Fv fragment (scFv)). In embodiments, the anti-TCRβV antibody molecule antibody molecule is a monoclonal antibody or an antibody with single specificity. In some embodiments, the anti-TCRβV antibody molecule can also be a humanized, chimeric, camelid, shark, or an in vitro-generated antibody molecule. In some embodiments, the anti-TCRβV antibody molecule is a humanized antibody molecule. The heavy and light chains of the anti-TCRβV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule is in the form of a multi-specific molecule, e.g., a bispecific molecule, e.g., as described herein.

### Anti-TCRβ V6 Antibodies

In some embodiments, the anti-TCRβV antibody molecule binds to human TCRβ V6, e.g., a TCRβ V6 subfamily comprising: TCRβ V6-4*01, TCRβ V6-4*02, TCRβ V6-9*01, TCRβ V6-8*01, TCRβ V6-5*01, TCRβ V6-6*02, TCRβ V6-6*01, TCRβ V6-2*01, TCRβ V6-3*01 or TCRβ V6-1*01. In some embodiments the TCRβ V6 subfamily comprises TCRβ V6-5*01.

In some embodiments, the anti-TCRβV antibody molecule, e.g., anti-TCRβ V6 (e.g., anti-TCRβ V6-5*01) antibody molecule, is a non-murine antibody molecule, e.g., a human or humanized antibody molecule. In some embodiments, the anti-TCRβV antibody molecule, e.g., anti-TCRβ V6 (e.g., anti-TCRβ V6-5*01) antibody molecule is a human antibody molecule. In some embodiments, the anti-TCRβV antibody molecule, e.g., anti-TCRβ V6 (e.g., anti-TCRβ V6-5*01) antibody molecule is a humanized antibody molecule.

In some embodiments, the anti-TCRβV antibody molecule, e.g., anti-TCRβ V6 (e.g., anti-TCRβ V6-5*01) antibody molecule, is isolated or recombinant.

In some embodiments, the anti-TCRβV antibody molecule, e.g., anti-TCRβ V6 (e.g., anti-TCRβ V6-5*01) antibody molecule, comprises at least one antigen-binding region, e.g., a variable region or an antigen-binding fragment thereof, from an antibody described herein, e.g., as described in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one, two, three or four variable regions from an antibody described herein, e.g., as described in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two heavy chain variable regions from an antibody described herein, e.g., as described in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises at least one or two light chain variable regions from an antibody described herein, e.g., as described in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, comprises a heavy chain constant region for an IgG4, e.g., a human IgG4. In still another embodiment, the anti-TCRβV antibody molecule, e.g., anti-TCRβ V6 (e.g., anti-TCRβ V6-5*01) antibody molecule includes a heavy chain constant region for an IgG1, e.g., a human IgG1. In one embodiment, the heavy chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes a kappa light chain constant region, e.g., a human kappa light chain constant region. In one embodiment, the light chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a heavy chain variable region of an antibody described herein, e.g., an antibody chosen from BHM1709 or BHM1710, or as described in Table 2, or encoded by the nucleotide sequence in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a heavy chain variable region comprising an amino acid sequence shown in Table 2, or encoded by a nucleotide sequence shown in Table 2. In one embodiment, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V6 (e.g., anti-TCRβ V6-5*01) antibody molecule, includes at least one, two, or three complementarity determining regions (CDRs) from a light chain variable region of an antibody described herein, e.g., an antibody as described in Table 2, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V6 (e.g., anti-TCRβ V6-5*01) antibody molecule, includes at least one, two, or three CDRs (or collectively all of the CDRs) from a light chain variable region comprising an amino acid sequence shown in Table 2, or encoded by a nucleotide sequence shown in Table 2. In one embodiment, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V6 (e.g., anti-TCRβ V6-5*01) antibody molecule, includes at least one, two, three, four, five or six CDRs (or collectively all of the CDRs) from a heavy and light chain variable region comprising an amino acid sequence shown in Table 2, or encoded by a nucleotide sequence shown in Table 2. In one embodiment, one or more of the CDRs (or collectively all of the CDRs) have one, two, three, four, five, six or more changes, e.g., amino acid substitutions or deletions, relative to the amino acid sequence shown in Table 2, or encoded by a nucleotide sequence shown in Table 2.

In some embodiments, the anti-TCRβV antibody molecule, e.g., anti-TCRβ V6 (e.g., anti-TCRβ V6-5*01) antibody molecule, molecule includes all six CDRs from an antibody described herein, e.g., as described in Table 2, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 2) from a heavy chain variable region of an antibody described herein, e.g., as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V6 (e.g., anti-TCRβ V6-5*01) antibody molecule includes at least one, two, or three CDRs according to Kabat et al. (e.g., at least one, two, or three CDRs according to the Kabat definition as set out in Table 2) from a light chain variable region of an antibody described herein, e.g., as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 2.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Kabat et al. (e.g., at least one, two, three, four, five, or six CDRs according to the Kabat definition as set out in Table 2) from the heavy and light chain variable regions of an antibody described herein, e.g., as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 2.

In some embodiments, the anti-TCRβV antibody molecule, e.g., anti-TCRβ V6 (e.g., anti-TCRβ V6-5*01) antibody molecule, includes all six CDRs according to Kabat et al. (e.g., all six CDRs according to the Kabat definition as set out in Table 2) from the heavy and light chain variable regions of an antibody described herein, e.g., or as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 2. In one embodiment, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, includes at least one, two, or three hypervariable loops that have the same canonical structures as the corresponding hypervariable loop of an antibody described herein, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 2) from a heavy chain variable region of an antibody described herein, e.g., as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V6 (e.g., anti-TCRβ V6-5*01) antibody molecule includes at least one, two, or three CDRs according to Chothia et al. (e.g., at least one, two, or three CDRs according to the Chothia definition as set out in Table 2) from a light chain variable region of an antibody described herein, e.g., as described in Table 2, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 2.

In some embodiments, the anti-TCRβV antibody molecule, e.g., anti-TCRβ V6 (e.g., anti-TCRβ V6-5*01) antibody molecule, includes at least one, two, three, four, five, or six CDRs according to Chothia et al. (e.g., at least one, two, three, four, five, or six CDRs according to the Chothia definition as set out in Table 2) from the heavy and light chain variable regions of an antibody described herein, e.g., as described in Table 2, or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 2.

In some embodiments, the anti-TCRβV antibody molecule, e.g., anti-TCRβ V6 (e.g., anti-TCRβ V6-5*01) antibody molecule, includes all six CDRs according to Chothia et al. (e.g., all six CDRs according to the Chothia definition as set out in Table 8) from the heavy and light chain variable regions of an antibody described herein, e.g., as described in Table 2, or encoded by the nucleotide sequence in Table 2; or encoded by the nucleotide sequence in Table 2; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 2. In one embodiment, the anti-TCRβV antibody molecule, e.g., anti-TCRβ V6 (e.g., anti-TCRβ V6-5*01) antibody molecule, may include any CDR described herein.

In some embodiments, the anti-TCRβV antibody molecule, e.g., anti-TCRβ V6 (e.g., anti-TCRβ V6-5*01) antibody molecule, molecule includes a combination of CDRs or hypervariable loops defined according to Kabat et al., Chothia et al., or as described in Table 2.

In some embodiments, the anti-TCRβV antibody molecule, e.g., anti-TCRβ V6 (e.g., anti-TCRβ V6-5*01) antibody molecule, can contain any combination of CDRs or hypervariable loops according to the Kabat and Chothia definitions.

In some embodiments, a combined CDR as set out in Table 2 is a CDR that comprises a Kabat CDR and a Chothia CDR.

In some embodiments, the anti-TCRβV antibody molecule, e.g., anti-TCRβ V6 (e.g., anti-TCRβ V6-5*01) antibody molecule, molecule includes a combination of CDRs or hypervariable loops identified as combined CDRs in Table 2. In some embodiments, the anti-TCRβV antibody molecule, e.g., anti-TCRβ V6 (e.g., anti-TCRβ V6-5*01) antibody molecule, can contain any combination of CDRs or hypervariable loops according the “combined” CDRs are described in Table 2.

In an embodiment, e.g., an embodiment comprising a variable region, a CDR (e.g., a combined CDR, Chothia CDR or Kabat CDR), or other sequence referred to herein, e.g., in Table 2, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprise a multi-specific molecule, e.g., a bispecific molecule, e.g., as described herein.

In an embodiment, the anti-TCRβV antibody molecule, e.g., anti-TCRβ V6 (e.g., anti-TCRβ V6-5*01) antibody molecule includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 10, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 11.

In some embodiments, the anti-TCRβV antibody molecule, e.g., anti-TCRβ V6 (e.g., anti-TCRβ V6-5*01) antibody molecule comprises a LC CDR1, LC CDR2, and LC CDR3 of SEQ ID NO: 10, and a HC CDR1, HC CDR2, and HC CDR3 of SEQ ID NO: 11.

In one embodiment, the light or the heavy chain variable framework (e.g., the region encompassing at least FR1, FR2, FR3, and optionally FR4) of the anti-TCRβV antibody molecule, e.g., anti-TCRβ V6 (e.g., anti-TCRβ V6-5*01) antibody molecule can be chosen from: (a) a light or heavy chain variable framework including at least 80%, 85%, 87% 90%, 92%, 93%, 95%, 97%, 98%, or 100% of the amino acid residues from a human light or heavy chain variable framework, e.g., a light or heavy chain variable framework residue from a human mature antibody, a human germline sequence, or a human consensus sequence; (b) a light or heavy chain variable framework including from 20% to 80%, 40% to 60%, 60% to 90%, or 70% to 95% of the amino acid residues from a human light or heavy chain variable framework, e.g., a light or heavy chain variable framework residue from a human mature antibody, a human germline sequence, or a human consensus sequence; (c) a non-human framework (e.g., a rodent framework); or (d) a non-human framework that has been modified, e.g., to remove antigenic or cytotoxic determinants, e.g., deimmunized, or partially humanized. In one embodiment, the light or heavy chain variable framework region (particularly FR1, FR2 and/or FR3) includes a light or heavy chain variable framework sequence at least 70, 75, 80, 85, 87, 88, 90, 92, 94, 95, 96, 97, 98, 99% identical or identical to the frameworks of a VL or VH segment of a human germline gene.

In some embodiments, the antibody comprises a heavy chain that shares at least 80%, 85%, 90%, 92%, 95%, 97%, 98%, 99%, or 100% identity to SEQ ID Nos: 19-21; the antibody comprises a light chain that shares at least 80%, 85%, 90%, 92%, 95%, 97%, 98%, 99%, or 100% identity to SEQ ID Nos: 22-23. In some embodiments, the antibody comprises a single chain Fv that shares at least 80%, 85%, 90%, 92%, 95%, 97%, 98%, 99%, or 100% identity to SEQ ID Nos: 24-48.

TABLE 2
Amino acid and nucleotide sequences for murine, chimeric and
humanized antibody molecules. The antibody molecules include
murine monoclonal antibody H131, several humanized versions
of H131, and several scFvs using humanized versions of H131.
H131 (murine)
Binds to human TCRVβ 6-5

| | | |
|---|---|---|
| SEQ ID NO: 1 | HC CDR1 (Combined) | GYSFTTYYIH |
| SEQ ID NO: 2 | HC CDR2 (Combined) | WFFPGSGNIKYNEKFKG |
| SEQ ID NO: 3 | HC CDR3 (Combined) | SYYSYDVLDY |
| SEQ ID NO: 4 | HC CDR1 (Kabat) | TYYIE |
| SEQ ID NO: 2 | HC CDR2 (Kabat) | WFFPGSGNIKYNEKFKG |
| SEQ ID NO: 3 | HC CDR3 (Kabat) | SYYSYDVLDY |
| SEQ ID NO: 5 | HC CDR1 (Chothia) | GYSFTTY |
| SEQ ID NO: 6 | HC CDR2 (Chothia) | FPGSGN |
| SEQ ID NO: 3 | HC CDR3 (Chothia) | SYYSYDVLDY |
| SEQ ID NO: 7 | LC CDR1 (Combined) | KASQNVGINVV |
| SEQ ID NO: 8 | LC CDR2 (Combined) | SSSHRYS |
| SEQ ID NO: 9 | LC CDR3 (Combined) | QQFKSYPLT |
| SEQ ID NO: 7 | LC CDR1 (Kabat) | KASQNVGINVV |
| SEQ ID NO: 8 | LC CDR2 (Kabat) | SSSHRYS |
| SEQ ID NO: 9 | LC CDR3 (Kabat) | QQFKSYPLT |
| SEQ ID NO: 7 | LC CDR1 (Chothia) | KASQNVGINVV |
| SEQ ID NO: 8 | LC CDR2 (Chothia) | SSSHRYS |
| SEQ ID NO: 9 | LC CDR3 (Chothia) | QQFKSYPLT |
| SEQ ID NO: 10 | VL | DILMTQSQKFMSTSLGDRVSVSCKASQNVGINVVW |
| | | HQQKPGQSPKALIYSSSHRYSGVPDRFTGSGSGTD |
| | | FTLTINNVQSEDLAEYFCQQFKSYPLTFGAGTKLE |
| | | LK |
| SEQ ID NO: 11 | VH | QVQLQQSGPELVKPGTSVKISCKASGYSFTTYYIE |
| | | WVKQRPGQGLEWIGWFFPGSGNIKYNEKFKGKATL |
| | | TADTSSSTAYMQLSSLTSEESAVYFCAGSYYSYDV |
| | | LDYWGHGTTLTVSS |

BHM1709 (humanized) Also referred to herein as TRVβ 6-5 v1, and
BJM0816 Binds to human TCRVβ 6-5

| | | |
|---|---|---|
| SEQ ID NO: 1 | HC CDR1 (Combined) | GYSFTTYYIH |
| SEQ ID NO: 2 | HC CDR2 (Combined) | WFFPGSGNIKYNEKFKG |
| SEQ ID NO: 3 | HC CDR3 (Combined) | SYYSYDVLDY |
| SEQ ID NO: 4 | HC CDR1 (Kabat) | TYYIE |
| SEQ ID NO: 2 | HC CDR2 (Kabat) | WFFPGSGNIKYNEKFKG |
| SEQ ID NO: 3 | HC CDR3 (Kabat) | SYYSYDVLDY |
| SEQ ID NO: 5 | HC CDR1 (Chothia) | GYSFTTY |
| SEQ ID NO: 6 | HC CDR2 (Chothia) | FPGSGN |
| SEQ ID NO: 3 | HC CDR3 (Chothia) | SYYSYDVLDY |
| SEQ ID NO: 7 | LC CDR1 (Combined) | KASQNVGINVV |
| SEQ ID NO: 8 | LC CDR2 (Combined) | SSSHRYS |
| SEQ ID NO: 9 | LC CDR3 (Combined) | QQFKSYPLT |
| SEQ ID NO: 7 | LC CDR1 (Kabat) | KASQNVGINVV |
| SEQ ID NO: 8 | LC CDR2 (Kabat) | SSSHRYS |
| SEQ ID NO: 9 | LC CDR3 (Kabat) | QQFKSYPLT |
| SEQ ID NO: 7 | LC CDR1 (Chothia) | KASQNVGINVV |
| SEQ ID NO: 8 | LC CDR2 (Chothia) | SSSHRYS |
| SEQ ID NO: 9 | LC CDR3 (Chothia) | QQFKSYPLT |
| SEQ ID NO: 12 | VH | QVQLVQSGAEVKKPGSSVKVSCKASGYSFTTYYIE |
| | | WVRQAPGQGLEWMGWFFPGSGNIKYNEKFKGRVTI |
| | | TADTSTSTAYMELSSLRSEDTAVYYCAGSYYSYDV |
| | | LDYWGQGTTVTVSS |
| SEQ ID NO: 13 | DNA VH | CAGGTGCAGCTGGTTCAGTCTGGCGCCGAAGTGAA |
| | | GAAACCTGGCTCCTCCGTGAAGGTGTCCTGCAAGG |
| | | CTTCCGGCTACTCCTTCACCACCTACTACATCCAC |
| | | TGGGTCCGACAGGCCCCTGGACAAGGATTGGAATG |
| | | GATGGGCTGGTTCTTCCCCGGCTCCGGCAACATCA |
| | | AGTACAACGAGAAGTTCAAGGGCCGCGTGACCATC |
| | | ACCGCCGACACCTCTACCTCTACCGCCTACATGGA |
| | | ACTGTCCAGCCTGAGATCTGAGGACACCGCCGTGT |
| | | ACTACTGCGCCGGCTCCTACTACTCTTACGACGTG |
| | | CTGGATTACTGGGGCCAGGGCACCACAGTGACAGT |
| | | GTCCTCT |
| SEQ ID NO: 14 | VL | DIQMTQSPSFLSASVGDRVTITCKASQNVGINVVW |
| | | HQQKPGKAPKALIYSSSHRYSGVPSRFSGSGSGTE |
| | | FTLTISSLQPEDFATYFCQQFKSYPLTFGQGTKLE |
| | | IK |
| SEQ ID NO: 15 | DNA VL | GACATCCAGATGACCCAGTCTCCATCCTTCCTGTC |
| | | CGCCTCTGTGGGCGACAGAGTGACCATCACATGCA |
| | | AGGCCTCTCAGAACGTGGGCATCAACGTCGTGTGG |
| | | CACCAGCAGAAGCCTGGCAAGGCTCCTAAGGCTCT |
| | | GATCTACTCCTCCAGCCACCGGTACTCTGGCGTGC |
| | | CCTCTAGATTTTCCGGCTCTGGCTCTGGCACCGAG |
| | | TTTACCCTGACAATCTCCAGCCTGCAGCCTGAGGA |
| | | CTTCGCCACCTACTTTTGCCAGCAGTTCAAGAGCT |
| | | ACCCTCTGACCTTTGGCCAGGGCACCAAGCTGGAA |
| | | ATCAAG |

BHM1710 (humanized) Also referred to herein as TRVβ 6-5 v2
Binds to human TCRVβ 6-5

| | | |
|---|---|---|
| SEQ ID NO: 1 | HC CDR1 (Combined) | GYSFTTYYIH |
| SEQ ID NO: 2 | HC CDR2 (Combined) | WFFPGSGNIKYNEKFKG |
| SEQ ID NO: 3 | HC CDR3 (Combined) | SYYSYDVLDY |
| SEQ ID NO: 4 | HC CDR1 (Kabat) | TYYIE |
| SEQ ID NO: 2 | HC CDR2 (Kabat) | WFFPGSGNIKYNEKFKG |
| SEQ ID NO: 3 | HC CDR3 (Kabat) | SYYSYDVLDY |
| SEQ ID NO: 5 | HC CDR1 (Chothia) | GYSFTTY |
| SEQ ID NO: 6 | HC CDR2 (Chothia) | FPGSGN |
| SEQ ID NO: 3 | HC CDR3 (Chothia) | SYYSYDVLDY |
| SEQ ID NO: 7 | LC CDR1 (Combined) | KASQNVGINVV |
| SEQ ID NO: 8 | LC CDR2 (Combined) | SSSHRYS |
| SEQ ID NO: 9 | LC CDR3 (Combined) | QQFKSYPLT |
| SEQ ID NO: 7 | LC CDR1 (Kabat) | KASQNVGINVV |
| SEQ ID NO: 8 | LC CDR2 (Kabat) | SSSHRYS |
| SEQ ID NO: 9 | LC CDR3 (Kabat) | QQFKSYPLT |
| SEQ ID NO: 7 | LC CDR1 (Chothia) | KASQNVGINVV |
| SEQ ID NO: 8 | LC CDR2 (Chothia) | SSSHRYS |
| SEQ ID NO: 9 | LC CDR3 (Chothia) | QQFKSYPLT |
| SEQ ID NO: 12 | VH | QVQLVQSGAEVKKPGSSVKVSCKASGYSFTTYYIE |
| | | WVRQAPGQGLEWMGWFFPGSGNIKYNEKFKGRVTI |
| | | TADTSTSTAYMELSSLRSEDTAVYYCAGSYYSYDV |
| | | LDYWGQGTTVTVSS |
| SEQ ID NO: 13 | DNA VH | CAGGTGCAGCTGGTTCAGTCTGGCGCCGAAGTGAA |
| | | GAAACCTGGCTCCTCCGTGAAGGTGTCCTGCAAGG |
| | | CTTCCGGCTACTCCTTCACCACCTACTACATCCAC |
| | | TGGGTCCGACAGGCCCCTGGACAAGGATTGGAATG |
| | | GATGGGCTGGTTCTTCCCCGGCTCCGGCAACATCA |
| | | AGTACAACGAGAAGTTCAAGGGCCGCGTGACCATC |
| | | ACCGCCGACACCTCTACCTCTACCGCCTACATGGA |
| | | ACTGTCCAGCCTGAGATCTGAGGACACCGCCGTGT |
| | | ACTACTGCGCCGGCTCCTACTACTCTTACGACGTG |
| | | CTGGATTACTGGGGCCAGGGCACCACAGTGACAGT |
| | | GTCCTCT |
| SEQ ID NO: 16 | VL | DIQMTQSPSSLSASVGDRVTITCKASQNVGINVVW |
| | | HQQKPGKVPKALIYSSSHRYSGVPSRFSGSGSGTD |
| | | FTLTISSLQPEDVATYFCQQFKSYPLTFGQGTKLE |
| | | IK |
| SEQ ID NO: 17 | DNA VL | GACATCCAGATGACCCAGTCTCCATCCTCTCTGTC |
| | | CGCCTCTGTGGGCGACAGAGTGACCATCACATGCA |
| | | AGGCCTCTCAGAACGTGGGCATCAACGTCGTGTGG |
| | | CACCAGCAGAAACCTGGCAAGGTGCCCAAGGCTCT |
| | | GATCTACTCCTCCAGCCACAGATACTCCGGCGTGC |
| | | CCTCTAGATTCTCCGGCTCTGGCTCTGGCACCGAC |
| | | TTTACCCTGACAATCTCCAGCCTGCAGCCTGAGGA |
| | | CGTGGCCACCTACTTTTGCCAGCAGTTCAAGAGCT |
| | | ACCCTCTGACCTTTGGCCAGGGCACCAAGCTGGAA |
| | | ATCAAG |

H131-3 (humanized)
Binds to human TCRVβ 6-5

| | | |
|---|---|---|
| SEQ ID NO: 1 | HC CDR1 (Combined) | GYSFTTYYIH |
| SEQ ID NO: 2 | HC CDR2 (Combined) | WFFPGSGNIKYNEKFKG |
| SEQ ID NO: 3 | HC CDR3 (Combined) | SYYSYDVLDY |
| SEQ ID NO: 4 | HC CDR1 (Kabat) | TYYIE |
| SEQ ID NO: 2 | HC CDR2 (Kabat) | WFFPGSGNIKYNEKFKG |
| SEQ ID NO: 3 | HC CDR3 (Kabat) | SYYSYDVLDY |
| SEQ ID NO: 5 | HC CDR1 (Chothia) | GYSFTTY |
| SEQ ID NO: 6 | HC CDR2 (Chothia) | FPGSGN |
| SEQ ID NO: 3 | HC CDR3 (Chothia) | SYYSYDVLDY |
| SEQ ID NO: 7 | LC CDR1 (Combined) | KASQNVGINVV |
| SEQ ID NO: 8 | LC CDR2 (Combined) | SSSHRYS |
| SEQ ID NO: 9 | LC CDR3 (Combined) | QQFKSYPLT |
| SEQ ID NO: 7 | LC CDR1 (Kabat) | KASQNVGINVV |
| SEQ ID NO: 8 | LC CDR2 (Kabat) | SSSHRYS |
| SEQ ID NO: 9 | LC CDR3 (Kabat) | QQFKSYPLT |
| SEQ ID NO: 7 | LC CDR1 (Chothia) | KASQNVGINVV |
| SEQ ID NO: 8 | LC CDR2 (Chothia) | SSSHRYS |
| SEQ ID NO: 9 | LC CDR3 (Chothia) | QQFKSYPLT |
| SEQ ID NO: 12 | VH | QVQLVQSGAEVKKPGSSVKVSCKASGYSFTTYYIE |
| | | WVRQAPGQGLEWMGWFFPGSGNIKYNEKFKGRVTI |
| | | TADTSTSTAYMELSSLRSEDTAVYYCAGSYYSYDV |
| | | LDYWGQGTTVTVSS |
| SEQ ID NO: 18 | VL | QSVLTQPPSVSEAPRQRVTISCKASQNVGINVVWH |
| | | QQLPGKAPKALIYSSSHRYSGVSDRFSGSGSGTSF |
| | | SLAISGLQSEDEADYFCQQFKSYPLTFGTGTKVTV |
| | | L |

H131 (humanized-matured) VHs
Binds to human TCRVβ 6-5

| | | |
|---|---|---|
| SEQ ID NO: 19 | VH-1 | QVQLVQSGAEVKKPGSSVKVSCKASGTDFKLTYIH |
| | | WVRQAPGQGLEWMGRIFPGSGNVKYNEKFKGRVTI |
| | | TADTSTSTAYMELSSLRSEDTAVYYCAGSYYSYDV |
| | | LDYWGQGTTVTVSS |
| SEQ ID NO: 20 | VH-2 | QVQLVQSGAEVKKPGSSVKVSCKASGTDFKLTYIH |
| | | WVRQAPGQGLEWMGRIFPGSGNVKYNEKFKGRVTI |
| | | TADTSTSTAYMELSSLRSEDTAVYYCAVSYYSYDV |
| | | LDYWGQGTTVTVSS |
| SEQ ID NO: 21 | VH-3 | QVQLVQSGAEVKKPGSSVKVSCKASGHDFRLTYIH |
| | | WVRQAPGQGLEWMGRISAGSGNVKYNEKFKGRVTI |
| | | TADTSTSTAYMELSSLRSEDTAVYYCAVSYYSYDV |
| | | LDYWGQGTTVTVSS |

H131 (humanized-matured) VLs
Binds to human TCRVβ 6-5

| | | |
|---|---|---|
| SEQ ID NO: 22 | VL-1 | DIQMTQSPSFLSASVGDRVTITCKASQNVDNRVAW |
| | | YQQKPGKAPKALIYSSSHRYKGVPSRFSGSGSGTE |
| | | FTLTISSLQPEDFATYFCQQFKSYPLTFGQGTKLE |
| | | IK |
| SEQ ID NO: 23 | VL-2 | DIQMTQSPSFLSASVGDRVTITCKASQNVADRVAW |
| | | YQQKPGKAPKALIYSSSHRYKGVPSRFSGSGSGTE |
| | | FTLTISSLQPEDFATYFCQQFKSYPLTFGQGTKLE |
| | | IK |

H131 scFv' s
Binds to human TCRVβ 6-5

| | | |
|---|---|---|
| SEQ ID NO: 24 | ScFv-1 | QVQLVQSGAEVKKPGSSVKVSCKASGTDFDKIYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVEDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 25 | ScFv-2 | QVQLVQSGAEVKKPGSSVKVSCKASGHDFRDFYIH |
| | | WVRQAPGQGLEWMGRVYPGSGSYRYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 26 | ScFv-3 | QVQLVQSGAEVKKPGSSVKVSCKASGHDFKLTYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDNRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 27 | ScFv-4 | QVQLVQSGAEVKKPGSSVKVSCKASGTDFKLTYIH |
| | | WVRQAPGQGLEWMGRIFPGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVENKVAWH |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 28 | ScFv-5 | QVQLVQSGAEVKKPGSSVKVSCKASGHDFDKFYIH |
| | | WVRQAPGQGLEWMGRVSAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGNRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 29 | ScFv-6 | QVQLVQSGAEVKKPGSSVKVSCKASGHDFDKFYIH |
| | | WVRQAPGQGLEWMGRIFAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 30 | ScFv-7 | QVQLVQSGAEVKKPGSSVKVSCKASGTDFKLTYIH |
| | | WVRQAPGQGLEWMGRVSPGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 31 | ScFv-8 | QVQLVQSGAEVKKPGSSVKVSCKASGTDFKLTYIH |
| | | WVRQAPGQGLEWMGRIFPGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDNRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 32 | ScFv-9 | QVQLVQSGAEVKKPGSSVKVSCKASGGTFRLTYIR |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 33 | ScFv-10 | QVQLVQSGAEVKKPGSSVKVSCKASGTDFKLTYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDNKVAWH |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 34 | ScFv-11 | QVQLVQSGAEVKKPGSSVKVSCKASGTDFKLTYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDNKVAWH |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 35 | ScFv-12 | QVQLVQSGAEVKKPGSSVKVSCKASGHDFRLTYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVADRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 36 | ScFv-13 | QVQLVQSGAEVKKPGSSVKVSCKASGHDFHLWYIH |
| | | WVRQAPGQGLEWMGRVSAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDNKVAWH |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 37 | ScFv-14 | QVQLVQSGAEVKKPGSSVKVSCKASGTDFKLTYIH |
| | | WVRQAPGQGLEWMGRVSAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGNRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 38 | ScFv-15 | QVQLVQSGAEVKKPGSSVKVSCKASGHDFHLWYIH |
| | | WVRQAPGQGLEWMGRISPGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGDRVAWH |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 39 | ScFv-16 | QVQLVQSGAEVKKPGSSVKVSCKASGHDFKLTYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 40 | ScFv-17 | QVQLVQSGAEVKKPGSSVKVSCKASGTDFHLWYIH |
| | | WVRQAPGQGLEWMGRVFAGSGSYRYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 41 | ScFv-18 | QVQLVQSGAEVKKPGSSVKVSCKASGHDFDKTYIH |
| | | WVRQAPGQGLEWMGRVSAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVEDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 42 | ScFv-19 | QVQLVQSGAEVKKPGSSVKVSCKASGHDFDKTYIH |
| | | WVRQAPGQGLEWMGRIYPGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVADRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 43 | ScFv-20 | QVQLVQSGAEVKKPGSSVKVSCKASGTDFDKTYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 44 | ScFv-21 | QVQLVQSGAEVKKPGSSVKVSCKASGTDFDKIYIH |
| | | WVRQAPGQGLEWMGRISAGSGNIKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 45 | ScFv-22 | QVQLVQSGAEVKKPGSSVKVSCKASGTDFDKIYIH |
| | | WVRQAPGQGLEWMGRISAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 46 | ScFv-23 | QVQLVQSGAEVKKPGSSVKVSCKASGGTFKLTYIH |
| | | WVRQAPGQGLEWMGRVSAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDDRVAWY |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 47 | ScFv-24 | QVQLVQSGAEVKKPGSSVKVSCKASGTDFKLTYIH |
| | | WVRQAPGQGLEWMGRISPGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVDNRVAWH |
| | | QQKPGKAPKALIYSSSHRYKGVPSRFSGSGSGTEF |
| | | TLTISSLQPEDFATYFCQQFKSYPLTFGQGTKLEI |
| | | K |
| SEQ ID NO: 48 | ScFv-25 | QVQLVQSGAEVKKPGSSVKVSCKASGTDFDKFYIH |
| | | WVRQAPGQGLEWMGRVSAGSGNVKYNEKFKGRVTI |
| | | TADTSTSTAYMELSSLRSEDTAVYYCAGSYYSYDV |
| | | LDYWGQGTTVTVSSGGGGSGGGGSGGGGSGGGGSD |
| | | IQMTQSPSFLSASVGDRVTITCKASQNVGDRVVWY |
| | | QQKPGKAPKALIYSSSHRYKGVPSRFSGSGSGTEF |
| | | TLTISSLQPEDFATYFCQQFKSYPLTFGQGTKLEI |
| | | K |

### Anti-TCRβ V12 Antibodies

Accordingly, in one aspect, the disclosure provides an anti-TCRβV antibody molecule that binds to human TCRβ V12, e.g., a TCRβ V12 subfamily comprising: TCRβ V12-4*01, TCRβ V12-3*01 or TCRβ V12-5*01. In some embodiments the TCRβ V12 subfamily comprises TCRβ V12-4*01. In some embodiments the TCRβ V12 subfamily comprises TCRβ V12-3*01.

In some embodiments, the anti-TCRβV antibody molecule, e.g., anti-TCRβ V12 antibody molecule, is a non-murine antibody molecule, e.g., a human or humanized antibody molecule. In some embodiments, the anti-TCRβV antibody molecule, e.g., anti-TCRβ V12 antibody molecule, is a human antibody molecule. In some embodiments, the anti-TCRβV antibody molecule, e.g., anti-TCRβ V12 antibody molecule is a humanized antibody molecule.

In some embodiments, the anti-TCRβV antibody molecule, e.g., anti-TCRβ V12 antibody molecule, is isolated or recombinant.

In some embodiments, the anti-TCRβV antibody molecule, e.g., anti-TCRβ V12 antibody molecule, comprises at least one antigen-binding region, e.g., a variable region or an antigen-binding fragment thereof, from an antibody described herein, e.g., an antibody described in Table 3, or encoded by the nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one, two, three or four variable regions from an antibody described herein, e.g., an antibody as described in Table 3, or encoded by the nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two heavy chain variable regions from an antibody described herein, e.g., an antibody as described in Table 3, or encoded by the nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises at least one or two light chain variable regions from an antibody described herein, e.g., an antibody as described in Table 3, or encoded by the nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, comprises a heavy chain constant region for an IgG4, e.g., a human IgG4. In still another embodiment, the anti-TCRβV antibody molecule, e.g., anti-TCRβ V12 antibody molecule, includes a heavy chain constant region for an IgG1, e.g., a human IgG1. In one embodiment, the heavy chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes a kappa light chain constant region, e.g., a human kappa light chain constant region. In one embodiment, the light chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule, e.g., anti-TCRβ V12 antibody molecule, includes at least one, two, or three complementarity determining regions (CDRs) from a heavy chain variable region of an antibody described herein, e.g., an antibody as described in Table 3, or encoded by the nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a heavy chain variable region comprising an amino acid sequence shown in Table 3, or encoded by a nucleotide sequence shown in Table 3. In one embodiment, one or more of the CDRs (or collectively all of the CDRs) have one, two, three, four, five, six or more changes, e.g., amino acid substitutions or deletions, relative to the amino acid sequence shown in Table 3, or encoded by a nucleotide sequence shown in Table 3.

In some embodiments, the anti-TCRβV antibody molecule, e.g., anti-TCRβ V12 antibody molecule, includes at least one, two, or three complementarity determining regions (CDRs) from a light chain variable region of an antibody described herein, e.g., an antibody as described in Table 3, or encoded by the nucleotide sequence in Table 3, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule, e.g., anti-TCRβ V12 antibody molecule, includes at least one, two, or three CDRs (or collectively all of the CDRs) from a light chain variable region comprising an amino acid sequence shown in Table 3, or encoded by a nucleotide sequence shown in Table 3. In one embodiment, one or more of the CDRs (or collectively all of the CDRs) have one, two, three, four, five, six or more changes, e.g., amino acid substitutions or deletions, relative to the amino acid sequence shown in Table 3, or encoded by a nucleotide sequence shown in Table 3.

In some embodiments, the anti-TCRβV antibody molecule, e.g., anti-TCRβ V12 antibody molecule, includes at least one, two, three, four, five or six CDRs (or collectively all of the CDRs) from a heavy and light chain variable region comprising an amino acid sequence shown in Table 3, or encoded by a nucleotide sequence shown in Table 3. In one embodiment, one or more of the CDRs (or collectively all of the CDRs) have one, two, three, four, five, six or more changes, e.g., amino acid substitutions or deletions, relative to the amino acid sequence shown in Table 3, or encoded by a nucleotide sequence shown in Table 3.

In some embodiments, the anti-TCRβV antibody molecule, e.g., anti-TCRβ V12 antibody molecule, molecule includes all six CDRs from an antibody described herein, e.g., an antibody as described in Table 3, or encoded by the nucleotide sequence in Table 3, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule, includes at least one, two, or three CDRs according to Kabat et al. (e.g., at least one, two, or three CDRs according to the Kabat definition as set out in Table 3) from a heavy chain variable region of an antibody described herein, e.g., an antibody chosen as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 3.

In some embodiments, the anti-TCRβV antibody molecule, e.g., anti-TCRβ V12 antibody molecule, includes at least one, two, or three CDRs according to Kabat et al. (e.g., at least one, two, or three CDRs according to the Kabat definition as set out in Table 3) from a light chain variable region of an antibody described herein, e.g., an antibody as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 3.

In some embodiments, the anti-TCRβV antibody molecule, e.g., anti-TCRβ V12 antibody molecule, includes at least one, two, three, four, five, or six CDRs according to Kabat et al. (e.g., at least one, two, three, four, five, or six CDRs according to the Kabat definition as set out in Table 3) from the heavy and light chain variable regions of an antibody described herein, e.g., an antibody as described in Table 3, or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 3.

In some embodiments, the anti-TCRβV antibody molecule, e.g., anti-TCRβ V12 antibody molecule, includes all six CDRs according to Kabat et al. (e.g., all six CDRs according to the Kabat definition as set out in Table 9) from the heavy and light chain variable regions of an antibody described herein, e.g., an antibody as described in Table 3, or encoded by the nucleotide sequence in Table 3; or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 3. In some embodiments, the anti-TCRβV antibody molecule, e.g., anti-TCRβ V12 antibody molecule may include any CDR described herein.

In some embodiments, the anti-TCRβV antibody molecule, e.g., anti-TCRβ V12 antibody molecule, includes at least one, two, or three hypervariable loops that have the same canonical structures as the corresponding hypervariable loop of an antibody described herein, e.g., an antibody described in Table 3, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule, e.g., anti-TCRβ V12 antibody molecule, includes at least one, two, or three CDRs according to Chothia et al. (e.g., at least one, two, or three CDRs according to the Chothia definition as set out in Table 3) from a heavy chain variable region of an antibody described herein, e.g., an antibody chosen as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 3.

In some embodiments, the anti-TCRβV antibody molecule, e.g., anti-TCRβ V12 antibody molecule, includes at least one, two, or three CDRs according to Chothia et al. (e.g., at least one, two, or three CDRs according to the Chothia definition as set out in Table 3) from a light chain variable region of an antibody described herein, e.g., an antibody as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 3.

In some embodiments, the anti-TCRβV antibody molecule, e.g., anti-TCRβ V12 antibody molecule, includes at least one, two, three, four, five, or six CDRs according to Chothia et al. (e.g., at least one, two, three, four, five, or six CDRs according to the Chothia definition as set out in Table 3) from the heavy and light chain variable regions of an antibody described herein, e.g., an antibody as described in Table 3, or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 3.

In some embodiments, the anti-TCRβV antibody molecule, e.g., anti-TCRβ V12 antibody molecule, includes all six CDRs according to Chothia et al. (e.g., all six CDRs according to the Chothia definition as set out in Table 9) from the heavy and light chain variable regions of an antibody described herein, e.g., an antibody as described in Table 3, or encoded by the nucleotide sequence in Table 3; or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 3. In some embodiments, the anti-TCRβV antibody molecule, e.g., anti-TCRβ V12 antibody molecule, may include any CDR described herein.

In some embodiments, the anti-TCRβV antibody molecule, e.g., anti-TCRβ V12 antibody molecule, includes at least one, two, or three CDRs according to a combined CDR (e.g., at least one, two, or three CDRs according to the combined CDR definition as set out in Table 3) from a heavy chain variable region of an antibody described herein, e.g., an antibody chosen as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 3.

In some embodiments, the anti-TCRβV antibody molecule, e.g., anti-TCRβ V12 antibody molecule, includes at least one, two, or three CDRs according to a combined CDR (e.g., at least one, two, or three CDRs according to the combined CDR definition as set out in Table 3) from a light chain variable region of an antibody described herein, e.g., an antibody as described in Table 3, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 3.

In some embodiments, the anti-TCRβV antibody molecule, e.g., anti-TCRβ V12 antibody molecule, includes at least one, two, three, four, five, or six CDRs according to a combined CDR. (e.g., at least one, two, three, four, five, or six CDRs according to the combined CDR definition as set out in Table 3) from the heavy and light chain variable regions of an antibody described herein, e.g., an antibody as described in Table 3, or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 3.

In some embodiments, the anti-TCRβV antibody molecule, e.g., anti-TCRβ V12 antibody molecule, includes all six CDRs according to a combined CDR (e.g., all six CDRs according to the combined CDR definition as set out in Table 3) from the heavy and light chain variable regions of an antibody described herein, e.g., an antibody as described in Table 3, or encoded by the nucleotide sequence in Table 3; or encoded by the nucleotide sequence in Table 3; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 3. In some embodiments, the anti-TCRβV antibody molecule, e.g., anti-TCRβ V12 antibody molecule, may include any CDR described herein.

In some embodiments, the anti-TCRβV antibody molecule, e e.g., anti-TCRβ V12 antibody molecule, molecule includes a combination of CDRs or hypervariable loops identified as combined CDRs in Table 3. In some embodiments, the anti-TCRβV antibody molecule, e.g., anti-TCRβ V12 antibody molecule, can contain any combination of CDRs or hypervariable loops according the “combined” CDRs are described in Table 3.

In some embodiments, the anti-TCRβV antibody molecule, e.g., anti-TCRβ V12 antibody molecule, includes a combination of CDRs or hypervariable loops defined according to the Kabat et al. and Chothia et al., or as described in Table 3.

In some embodiments, the anti-TCRβV antibody molecule, e.g., anti-TCRβ V12 antibody molecule, can contain any combination of CDRs or hypervariable loops according to the Kabat and Chothia definitions.

In an embodiment, e.g., an embodiment comprising a variable region, a CDR (e.g., a combined CDR, Chothia CDR or Kabat CDR), or other sequence referred to herein, e.g., in Table 3, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprise a multispecific molecule, e.g., a bispecific molecule, e.g., as described herein.

In some embodiments, the anti-TCRβV antibody molecule, e.g., anti-TCRβ V12 antibody molecule, includes: (i) one, two or all of a light chain complementarity determining region 1 (LC CDR1), a light chain complementarity determining region 2 (LC CDR2), and a light chain complementarity determining region 3 (LC CDR3) of SEQ ID NO: 59, and/or (ii) one, two or all of a heavy chain complementarity determining region 1 (HC CDR1), heavy chain complementarity determining region 2 (HC CDR2), and a heavy chain complementarity determining region 3 (HC CDR3) of SEQ ID NO: 58.

In some embodiments, the heavy or light chain variable domain, or both, of, the anti-TCRβV antibody molecule, e.g., anti-TCRβ V12 antibody molecule, includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody as described in Table 3, or encoded by the nucleotide sequence in Table 3; or which differs at least 1 or 5 residues, but less than 40, 30, 20, or 10 residues, from a variable region of an antibody described herein.

In some embodiments, the anti-TCRβV antibody molecule, e.g., anti-TCRβ V12 antibody molecule, comprises at least one, two, three, or four antigen-binding regions, e.g., variable regions, having an amino acid sequence as set forth in Table 3, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 3. In another embodiment, the anti-TCRβV antibody molecule, e.g., anti-TCRβ V12 antibody molecule, includes a VH and/or VL domain encoded by a nucleic acid having a nucleotide sequence as set forth in Table 3, or a sequence substantially identical thereto (e.g., a sequence at least about 85%, 90%, 95%, 99% or more identical thereto, or which differs by no more than 3, 6, 15, 30, or 45 nucleotides from the sequences shown in Table 3.

In some embodiments, the anti-TCRβV antibody molecule, e.g., anti-TCRβ V12 antibody molecule, is a full antibody or fragment thereof (e.g., a Fab, F(ab′)2, Fv, or a single chain Fv fragment (scFv)). In embodiments, the anti-TCRβV antibody molecule, e.g., anti-TCRβ V6 (e.g., anti-TCRβ V6-5*01) antibody molecule, is a monoclonal antibody or an antibody with single specificity. In some embodiments, the anti-TCRβV antibody molecule, e.g., anti-TCRβ V12 antibody molecule, can also be a humanized, chimeric, camelid, shark, or an in vitro-generated antibody molecule. In some embodiments, the anti-TCRβV antibody molecule, e.g., anti-TCRβ V12 antibody molecule, is a humanized antibody molecule. The heavy and light chains of the anti-TCRβV antibody molecule, e.g., anti-TCRβ V12 antibody molecule, can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule, e.g., anti-TCRβ V12 antibody molecule, is in the form of a multispecific molecule, e.g., a bispecific molecule, e.g., as described herein.

In some embodiments, the anti-TCRβV antibody molecule, e.g., anti-TCRβ V12 antibody molecule, has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the anti-TCRβV antibody molecule does not bind to TCRβ V12, or binds to TCRβ V12 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to TCRβ V12 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule binds to a TCRβV region other than TCRβ V12 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the 16G8 murine antibody or a humanized version thereof as described in U.S. Pat. No. 5,861,155.

In some embodiments, the anti-TCRβV antibody molecule does not comprise the CDRs of the 16G8 murine antibody.

In some embodiments, the anti-TCRβV antibody molecule does not bind to TCRβ V5-5*01 or TCRβ V5-1*01, or binds to TCRβ V5-5*01 or TCRβ V5-1*01 with an affinity and/or binding specificity that is less than (e.g., less than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to TCRβ V5-5*01 or TCRβ V5-1*01 with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule binds to a TCRβV region other than TCRβ V5-5*01 or TCRβ V5-1*01 (e.g., TCRβV region as described herein, e.g., TCRβ V6 subfamily (e.g., TCRβ V6-5*01) with an affinity and/or binding specificity that is greater than (e.g., greater than about 10%, 20%, 30%, 40%, 50%, 60%, 70%, 80%, 90% or about 2-, 5-, or 10-fold) the affinity and/or binding specificity of the TM23 murine antibody or a humanized version thereof as described in U.S. Pat. No. 5,861,155.

In some embodiments, the anti-TCRβV antibody molecule does not comprise the CDRs of the TM23 murine antibody.

In some embodiments, the antibody comprises a heavy chain that shares at least 80%, 85%, 90%, 92%, 95%, 97%, 98%, 99%, or 100% identity to SEQ ID Nos: 63, 64, or 66; the antibody comprises a light chain that shares at least 80%, 85%, 90%, 92%, 95%, 97%, 98%, 99%, or 100% identity to SEQ ID Nos: 62, 65, or 67-69.

TABLE 3
Amino acid and nucleotide sequences for murine and humanized
antibody molecules. The antibody molecules include murine
monoclonal antibody 16G8 and several humanized versions of 16G8.
16G8 (murine) (also referred to as TM27)
Binds to human TCRVβ 12-3/12-4

| | | |
|---|---|---|
| SEQ ID NO: 49 | HC CDR1 (Combined) | GFTFSNFGMH |
| SEQ ID NO: 50 | HC CDR2 (Combined) | YISSGSSTIYYADTLKG |
| SEQ ID NO: 51 | HC CDR3 (Combined) | RGEGAMDY |
| SEQ ID NO: 52 | HC CDR1 (Kabat) | NFGMH |
| SEQ ID NO: 50 | HC CDR2 (Kabat) | YISSGSSTIYYADTLKG |
| SEQ ID NO: 51 | HC CDR3 (Kabat) | RGEGAMDY |
| SEQ ID NO: 53 | HC CDR1 (Chothia) | GFTFSNF |
| SEQ ID NO: 54 | HC CDR2 (Chothia) | SSGSST |
| SEQ ID NO: 51 | HC CDR3 (Chothia) | RGEGAMDY |
| SEQ ID NO: 55 | LC CDR1 (Combined) | RASSSVNYIY |
| SEQ ID NO: 56 | LC CDR2 (Combined) | YTSNLAP |
| SEQ ID NO: 57 | LC CDR3 (Combined) | QQFTSSPFT |
| SEQ ID NO: 55 | LC CDR1 (Kabat) | RASSSVNYIY |
| SEQ ID NO: 56 | LC CDR2 (Kabat) | YTSNLAP |
| SEQ ID NO: 57 | LC CDR3 (Kabat) | QQFTSSPFT |
| SEQ ID NO: 55 | LC CDR1 (Chothia) | RASSSVNYIY |
| SEQ ID NO: 56 | LC CDR2 (Chothia) | YTSNLAP |
| SEQ ID NO: 57 | LC CDR3 (Chothia) | QQFTSSPFT |
| SEQ ID NO: 58 | VH | EVQLVESGGGLVQPGGSRKLSCAASGFTFSNFGMHW |
| | | VRQAPGKGLEWVAYISSGSSTIYYADTLKGRFTISR |
| | | DNPKNTLFLQMTSLRSEDTAMYYCARRGEGAMDYWG |
| | | QGTSVTVSS |
| SEQ ID NO: 59 | VL | ENVLTQSPAIMSASLGEKVTMSCRASSSVNYIYQYQ |
| | | QKSDASPKLWIYYTSNLAPGVPTRFSGSGSGNSYSL |
| | | TISSMEGEDAATYYCQQFTSSPFTFGSGTKLEIK |

TM29 (humanized)
Binds human TCRBβ 12-3/12-4

| | | |
|---|---|---|
| SEQ ID NO: 49 | HC CDR1 (Combined) | GFTFSNFGMH |
| SEQ ID NO: 50 | HC CDR2 (Combined) | YISSGSSTIYYADTLKG |
| SEQ ID NO: 51 | HC CDR3 (Combined) | RGEGAMDY |
| SEQ ID NO: 52 | HC CDR1 (Kabat) | NFGMH |
| SEQ ID NO: 50 | HC CDR2 (Kabat) | YISSGSSTIYYADTLKG |
| SEQ ID NO: 51 | HC CDR3 (Kabat) | RGEGAMDY |
| SEQ ID NO: 53 | HC CDR1 (Chothia) | GFTFSNF |
| SEQ ID NO: 54 | HC CDR2 (Chothia) | SSGSST |
| SEQ ID NO: 51 | HC CDR3 (Chothia) | RGEGAMDY |
| SEQ ID NO: 55 | LC CDR1 (Combined) | RASSSVNYIY |
| SEQ ID NO: 56 | LC CDR2 (Combined) | YTSNLAP |
| SEQ ID NO: 57 | LC CDR3 (Combined) | QQFTSSPFT |
| SEQ ID NO: 55 | LC CDR1 (Kabat) | RASSSVNYIY |
| SEQ ID NO: 56 | LC CDR2 (Kabat) | YTSNLAP |
| SEQ ID NO: 57 | LC CDR3 (Kabat) | QQFTSSPFT |
| SEQ ID NO: 55 | LC CDR1 (Chothia) | RASSSVNYIY |
| SEQ ID NO: 56 | LC CDR2 (Chothia) | YTSNLAP |
| SEQ ID NO: 57 | LC CDR3 (Chothia) | QQFTSSPFT |
| SEQ ID NO: 60 | VL | DIQMTQSPSSLSASVGDRVTITCRASSSVNYIYWYQ |
| | | QTPGKAPKLLIYYTSNLAPGVPSRFSGSGSGTDYTF |
| | | TISSLQPEDIATYYCQQFTSSPFTFGQGTKLQIT |
| SEQ ID NO: 61 | VH | EVQLVESGGGVVQPGRSLRLSCSSGFTFSNFGMHWV |
| | | RQAPGKGLEWVAYISSGSSTIYYADTLKGRFTISRD |
| | | NSKNTLFLQMDSLRPEDTGVYFCARRGEGAMDYWGQ |
| | | G TPVTVSS |

16G8 (humanized −1)
Binds to human TCRV β 12-3/12-4

| | | |
|---|---|---|
| SEQ ID NO: 49 | HC CDR1 (Combined) | GFTFSNFGMH |
| SEQ ID NO: 50 | HC CDR2 (Combined) | YISSGSSTIYYADTLKG |
| SEQ ID NO: 51 | HC CDR3 (Combined) | RGEGAMDY |
| SEQ ID NO: 52 | HC CDR1 (Kabat) | NFGMH |
| SEQ ID NO: 50 | HC CDR2 (Kabat) | YISSGSSTIYYADTLKG |
| SEQ ID NO: 51 | HC CDR3 (Kabat) | RGEGAMDY |
| SEQ ID NO: 53 | HC CDR1 (Chothia) | GFTFSNF |
| SEQ ID NO: 54 | HC CDR2 (Chothia) | SSGSST |
| SEQ ID NO: 51 | HC CDR3 (Chothia) | RGEGAMDY |
| SEQ ID NO: 55 | LC CDR1 (Combined) | RASSSVNYIY |
| SEQ ID NO: 56 | LC CDR2 (Combined) | YTSNLAP |
| SEQ ID NO: 57 | LC CDR3 (Combined) | QQFTSSPFT |
| SEQ ID NO: 55 | LC CDR1 (Kabat) | RASSSVNYIY |
| SEQ ID NO: 56 | LC CDR2 (Kabat) | YTSNLAP |
| SEQ ID NO: 57 | LC CDR3 (Kabat) | QQFTSSPFT |
| SEQ ID NO: 55 | LC CDR1 (Chothia) | RASSSVNYIY |
| SEQ ID NO: 56 | LC CDR2 (Chothia) | YTSNLAP |
| SEQ ID NO: 57 | LC CDR3 (Chothia) | QQFTSSPFT |
| SEQ ID NO: 62 | VL | DNQLTQSPSFLSASVGDRVTITCRASSSVNYIYWYQ |
| | | QKPGKAPKLLIYYTSNLAPGVPSRFSGSGSGNEYTL |
| | | TISSLQPEDFATYYCQQFTSSPFTFGQGTKLEIK |
| SEQ ID NO: 63 | VH | QVQLVESGGGVVQPGRSLRLSCAASGFTFSNFGMHW |
| | | VRQAPGKGLEWVAYISSGSSTIYYADTLKGRFTISR |
| | | DNSKNTLYLQMNSLRAEDTAVYYCARRGEGAMDYWG |
| | | QGTTVTVSS |

16G8 (humanized −2) Also referred to herein as TCRVβ 12-3/4 v1 and
BHM1675 Binds human TCRVβ 12-3/12-4

| | | |
|---|---|---|
| SEQ ID NO: 62 | VL | DNQLTQSPSFLSASVGDRVTITCRASSSVNYIYWYQ |
| | | QKPGKAPKLLIYYTSNLAPGVPSRFSGSGSGNEYTL |
| | | TISSLQPEDFATYYCQQFTSSPFTFGQGTKLEIK |
| SEQ ID NO: 64 | VH | EVQLVESGGGLVQPGGSLRLSCAASGFTFSNFGMHW |
| | | VRQAPGKGLEWVSYISSGSSTIYYADTLKGRFTISR |
| | | DNSKNTLYLQMNSLRAEDTAVYYCARRGEGAMDYWG |
| | | QGTTVTVSS |

16G8 (humanized −3) Also referred to herein as TCRVβ 12-3/4 v2
Binds to human TCRVβ 12-3/12-4

| | | |
|---|---|---|
| SEQ ID NO: 62 | VL | DNQLTQSPSFLSASVGDRVTITCRASSSVNYIYWYQ |
| | | QKPGKAPKLLIYYTSNLAPGVPSRFSGSGSGNEYTL |
| | | TISSLQPEDFATYYCQQFTSSPFTFGQGTKLEIK |
| SEQ ID NO: 63 | VH | QVQLVESGGGVVQPGRSLRLSCAASGFTFSNFGMHW |
| | | VRQAPGKGLEWVAYISSGSSTIYYADTLKGRFTISR |
| | | DNSKNTLYLQMNSLRAEDTAVYYCARRGEGAMDYWG |
| | | QGTTVTVSS |

16G8 (humanized −4) Also referred to herein as TCRVβ 12-3/4 v3
Binds to human TCRVβ 12-3/12-4

| | | |
|---|---|---|
| SEQ ID NO: 65 | VL | DNQLTQSPSSLSASVGDRVTITCRASSSVNYIYWYQ |
| | | QKPGKAPKLLIYYTSNLAPGVPSRFSGSGSGNDYTL |
| | | TISSLQPEDFATYYCQQFTFSSPTFTGQGTKLEIK |
| SEQ ID NO: 63 | VH | QVLQVESGGGVVQPGRSLRLSCAASGFTFSNFGMHW |
| | | VRQAPGKGLEWVAYISSGSSTIYYADTLKGRFTISR |
| | | DNSKNTLYLQMNSLRAEDTAVYYCARRGEGAMDYWG |
| | | QGTTVTVSS |

16G8 (humanized) VHs
Binds to human TCRVβ 12-3/12-4

| | | |
|---|---|---|
| SEQ ID NO: 66 | VH-1 | EVQLVESGGGLVQPGGSLRLSCAASGFTFSNFGMHW |
| | | VRQAPGKGLEWVSYISSGSSTIYYADTLKGRFTISR |
| | | DNAKNSLYLQMNSLRAEDTAVYYCARRGEGAMDYWG |
| | | QGTTVTVSS |
| SEQ ID NO: 64 | VH-2 | EVQLVESGGGLVQPGGSLRLSCAASGFTFSNFGMHW |
| | | VRQAPGKGLEWVSYISSGSSTIYYADTLKGRFTISR |
| | | DNSKNTLYLQMNSLRAEDTAVYYCARRGEGAMDYWG |
| | | QGTTVTVSS |
| SEQ ID NO: 63 | VH-3 | QVQLVESGGGVVQPGRSLRLSCAASGFTFSNFGMHW |
| | | VRQAPGKGLEWVAYISSGSSTIYYADTLKGRFTISR |
| | | DNSKNTLYLQMNSLRAEDTAVYYCARRGEGAMDYWG |
| | | QGTTVTVSS |

16G8 (humanized) VLs
Binds to human TCRVβ 12-3/12-4

| | | |
|---|---|---|
| SEQ ID NO: 62 | VL-1 | DNQLTQSPSFLSASVGDRVTITCRASSSVNYIYWYQ |
| | | QKPGKAPKLLIYYTSNLAPGVPSRFSGSGSGNEYTL |
| | | TISSLQPEDFATYYCQQFTSSPFTFGQGTKLEIK |
| SEQ ID NO: 65 | VL-2 | DNQLTQSPSSLSASVGDRVTITCRASSSVNYIYWYQ |
| | | QKPGKAPKLLIYYTSNLAPGVPSRFSGSGSGNDYTL |
| | | TISSLQPEDFATYYCQQFTSSPFTFGQGTKLEIK |
| SEQ ID NO: 67 | VL-3 | ENVLTQSPATLSVSPGERATLSCRASSSVNYIYWYQ |
| | | QKPGQAPRLLIYYTSNLAPGIPARFSGSGSGNEYTL |
| | | TISSLQSEDFAVYYCQQFTSSPFTFGQGTKLEIK |
| SEQ ID NO: 68 | VL-4 | QNVLTQPPSASGTPGQRVTISVRASSSVNYIYWYQQ |
| | | LPGTAPKLLIYYTSNLAPGVPDRFSGSGSGNSYSLA |
| | | ISGLRSEDEADYYCQQFTSSPFTFGTGTKVTVL |
| SEQ ID NO: 69 | VL-5 | SNELTQPPSVSVSPGQTARITCRASSSVNYIYWYQQ |
| | | KSGQAPVLVIYYTSNLAPGIPERFSGSGSGNMYTLT |
| | | ISGAQVEDEADYYCQQFTSSPFTFGTGTKVTVL |

In some embodiments, the anti-TCRβV antibody molecule comprises at least one antigen-binding region, e.g., a variable region or an antigen-binding fragment thereof, from an antibody described herein, e.g., an antibody described in Table 4 or Table 5, or encoded by the nucleotide sequence in Table 4 or Table 5, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises at least one, two, three or four variable regions from an antibody described herein, e.g., an antibody as described in Table 4 or Table 5, or encoded by the nucleotide sequence in Table 4 or Table 5, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises at least one or two heavy chain variable regions from an antibody described herein, e.g., an antibody as described in Table 4 or Table 5, or encoded by the nucleotide sequence in Table 4 or Table 5, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises at least one or two light chain variable regions from an antibody described herein, e.g., an antibody as described in Table 4 or Table 5, or encoded by the nucleotide sequence in Table 4 or Table 5, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule comprises a heavy chain constant region for an IgG4, e.g., a human IgG4. In still another embodiment, the anti-TCRβV antibody molecule includes a heavy chain constant region for an IgG1, e.g., a human IgG1. In one embodiment, the heavy chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule includes a kappa light chain constant region, e.g., a human kappa light chain constant region. In one embodiment, the light chain constant region comprises an amino sequence set forth in Table 6, or a sequence substantially identical (e.g., at least 80%, 85%, 90%, 92%, 95%, 97%, 98%, 99% or higher identical) thereto.

In some embodiments, the anti-TCRβV antibody molecule includes at least one, two, or three complementarity determining regions (CDRs) from a heavy chain variable region of an antibody described herein, e.g., an antibody as described in Table 4 or Table 5, or encoded by the nucleotide sequence in Table 4 or Table 5, or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule includes at least one, two, or three CDRs (or collectively all of the CDRs) from a heavy chain variable region comprising an amino acid sequence shown in Table 4 or Table 5, or encoded by a nucleotide sequence shown in Table 4 or Table 5. In one embodiment, one or more of the CDRs (or collectively all of the CDRs) have one, two, three, four, five, six or more changes, e.g., amino acid substitutions or deletions, relative to the amino acid sequence shown in Table 4 or Table 5, or encoded by a nucleotide sequence shown in Table 4 or Table 5.

In some embodiments, the anti-TCRβV antibody molecule includes at least one, two, or three complementarity determining regions (CDRs) from a light chain variable region of an antibody described herein, e.g., an antibody as described in Table 4 or Table 5, or encoded by the nucleotide sequence in Table 4 or Table 5 or a sequence substantially identical (e.g., at least 80%, 85%, 90%, 92%, 95%, 97%, 98%, 99% or higher identical) to any of the aforesaid sequences.

In some embodiments, the anti-TCRβV antibody molecule includes at least one, two, or three CDRs (or collectively all of the CDRs) from a light chain variable region comprising an amino acid sequence shown in Table 4 or Table 5, or encoded by a nucleotide sequence shown in Table 4 or Table 5. In one embodiment, one or more of the CDRs (or collectively all of the CDRs) have one, two, three, four, five, six or more changes, e.g., amino acid substitutions or deletions, relative to the amino acid sequence shown in Table 4 or Table 5, or encoded by a nucleotide sequence shown in Table 4 or Table 5.

In some embodiments, the anti-TCRβV antibody molecule includes at least one, two, three, four, five or six CDRs (or collectively all of the CDRs) from a heavy and light chain variable region comprising an amino acid sequence shown in Table 4 or Table 5, or encoded by a nucleotide sequence shown in Table 4 or Table 5. In one embodiment, one or more of the CDRs (or collectively all of the CDRs) have one, two, three, four, five, six or more changes, e.g., amino acid substitutions or deletions, relative to the amino acid sequence shown in Table 4 or Table 5, or encoded by a nucleotide sequence shown in Table 4 or Table 5.

In some embodiments, the anti-TCRβV antibody molecule includes all six CDRs from an antibody described herein, e.g., an antibody as described in Table 4 or Table 5, or encoded by the nucleotide sequence in Table 4 or Table 5, or closely related CDRs, e.g., CDRs which are identical or which have at least one amino acid alteration, but not more than two, three or four alterations (e.g., substitutions, deletions, or insertions, e.g., conservative substitutions). In some embodiments, the anti-TCRβV antibody molecule may include any CDR described herein.

In some embodiments, the anti-TCRβV antibody molecule includes at least one, two, or three CDRs according to Kabat et al. (e.g., at least one, two, or three CDRs according to the Kabat definition as set out in Table 4 or Table 5) from a heavy chain variable region of an antibody described herein, e.g., an antibody chosen as described in Table 4 or Table 5, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 4 or Table 5.

In some embodiments, the anti-TCRβV antibody molecule includes at least one, two, or three CDRs according to Kabat et al. (e.g., at least one, two, or three CDRs according to the Kabat definition as set out in Table 4 or Table 5) from a light chain variable region of an antibody described herein, e.g., an antibody as described in Table 4 or Table 5, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Kabat et al. shown in Table 4 or Table 5.

In some embodiments, the anti-TCRβV antibody molecule includes at least one, two, three, four, five, or six CDRs according to Kabat et al. (e.g., at least one, two, three, four, five, or six CDRs according to the Kabat definition as set out in Table 4 or Table 5) from the heavy and light chain variable regions of an antibody described herein, e.g., an antibody as described in Table 4 or Table 5, or encoded by the nucleotide sequence in Table 4 or Table 5; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Kabat et al. shown in Table 4 or Table 5.

In some embodiments, the anti-TCRβV antibody molecule includes all six CDRs according to Kabat et al. (e.g., all six CDRs according to the Kabat definition as set out in Table 2) from the heavy and light chain variable regions of an antibody described herein, e.g., an antibody as described in Table 4 or Table 5, or encoded by the nucleotide sequence in Table 4 or Table 5; or encoded by the nucleotide sequence in Table 4 or Table 5; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Kabat et al. shown in Table 4 or Table 5. In some embodiments, the anti-TCRβV antibody molecule, e.g., anti-TCRβ V12 antibody molecule may include any CDR described herein.

In some embodiments, the anti-TCRβV antibody molecule includes at least one, two, or three hypervariable loops that have the same canonical structures as the corresponding hypervariable loop of an antibody described herein, e.g., an antibody described in Table 4 or Table 5, e.g., the same canonical structures as at least loop 1 and/or loop 2 of the heavy and/or light chain variable domains of an antibody described herein. See, e.g., Chothia et al., (1992) J. Mol. Biol. 227:799-817; Tomlinson et al., (1992) J. Mol. Biol. 227:776-798 for descriptions of hypervariable loop canonical structures. These structures can be determined by inspection of the tables described in these references.

In some embodiments, the anti-TCRβV antibody molecule includes at least one, two, or three CDRs according to Chothia et al. (e.g., at least one, two, or three CDRs according to the Chothia definition as set out in Table 4 or Table 5) from a heavy chain variable region of an antibody described herein, e.g., an antibody chosen as described in Table 4 or Table 5, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 4 or Table 5.

In some embodiments, the anti-TCRβV antibody molecule includes at least one, two, or three CDRs according to Chothia et al. (e.g., at least one, two, or three CDRs according to the Chothia definition as set out in Table 4 or Table 5) from a light chain variable region of an antibody described herein, e.g., an antibody as described in Table 4 or Table 5, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to Chothia et al. shown in Table 4 or Table 5.

In some embodiments, the anti-TCRβV antibody molecule includes at least one, two, three, four, five, or six CDRs according to Chothia et al. (e.g., at least one, two, three, four, five, or six CDRs according to the Chothia definition as set out in Table 4 or Table 5) from the heavy and light chain variable regions of an antibody described herein, e.g., an antibody as described in Table 4 or Table 5, or encoded by the nucleotide sequence in Table 4 or Table 5; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to Chothia et al. shown in Table 4 or Table 5.

In some embodiments, the anti-TCRβV antibody molecule includes all six CDRs according to Chothia et al. (e.g., all six CDRs according to the Chothia definition as set out in Table 9) from the heavy and light chain variable regions of an antibody described herein, e.g., an antibody as described in Table 4 or Table 5, or encoded by the nucleotide sequence in Table 4 or Table 5; or encoded by the nucleotide sequence in Table 4 or Table 5; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to Chothia et al. shown in Table 4 or Table 5. In some embodiments, the anti-TCRβV antibody molecule, e.g., anti-TCRβ V12 antibody molecule may include any CDR described herein.

In some embodiments, the anti-TCRβV antibody molecule includes at least one, two, or three CDRs according to a combined CDR (e.g., at least one, two, or three CDRs according to the combined CDR definition as set out in Table 4 or Table 5) from a heavy chain variable region of an antibody described herein, e.g., an antibody chosen as described in Table 4 or Table 5, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to combined CDR shown in Table 4 or Table 5.

In some embodiments, the anti-TCRβV antibody molecule includes at least one, two, or three CDRs according to a combined CDR (e.g., at least one, two, or three CDRs according to the combined CDR definition as set out in Table 4 or Table 5) from a light chain variable region of an antibody described herein, e.g., an antibody as described in Table 4 or Table 5, or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, or three CDRs according to a combined CDR shown in Table 4 or Table 5.

In some embodiments, the anti-TCRβV antibody molecule includes at least one, two, three, four, five, or six CDRs according to a combined CDR. (e.g., at least one, two, three, four, five, or six CDRs according to the combined CDR definition as set out in Table 4 or Table 5) from the heavy and light chain variable regions of an antibody described herein, e.g., an antibody as described in Table 4 or Table 5, or encoded by the nucleotide sequence in Table 4 or Table 5; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to one, two, three, four, five, or six CDRs according to a combined CDR shown in Table 4 or Table 5.

In some embodiments, the anti-TCRβV antibody molecule includes all six CDRs according to a combined CDR (e.g., all six CDRs according to the combined CDR definition as set out in Table 4 or Table 5) from the heavy and light chain variable regions of an antibody described herein, e.g., an antibody as described in Table 4 or Table 5, or encoded by the nucleotide sequence in Table 4 or Table 5; or encoded by the nucleotide sequence in Table 4 or Table 5; or a sequence substantially identical (e.g., at least 80%, 85%, 90%, 92%, 95%, 97%, 98%, 99% or higher identical) to any of the aforesaid sequences; or which have at least one amino acid alteration, but not more than two, three or four alterations (e.g., substitutions, deletions, or insertions, e.g., conservative substitutions) relative to all six CDRs according to a combined CDR shown in Table 4 or Table 5. In some embodiments, the anti-TCRβV antibody molecule may include any CDR described herein.

In some embodiments, the anti-TCRβV antibody molecule includes a combination of CDRs or hypervariable loops identified as combined CDRs in Table 4 or Table 5. In some embodiments, the anti-TCRβV antibody molecule contains any combination of CDRs or hypervariable loops according the “combined” CDRs are described in Table 4 or Table 5.

In some embodiments, the anti-TCRβV antibody molecule includes a combination of CDRs or hypervariable loops defined according to the Kabat et al. and Chothia et al., or as described in Table 4 or Table 5.

In some embodiments, the anti-TCRβV antibody molecule can contain any combination of CDRs or hypervariable loops according to the Kabat and Chothia definitions.

In an embodiment, e.g., an embodiment comprising a variable region, a CDR (e.g., a combined CDR, Chothia CDR or Kabat CDR), or other sequence referred to herein, e.g., in Table 4 or Table 5, the antibody molecule is a monospecific antibody molecule, a bispecific antibody molecule, a bivalent antibody molecule, a biparatopic antibody molecule, or an antibody molecule that comprises an antigen binding fragment of an antibody, e.g., a half antibody or antigen binding fragment of a half antibody. In certain embodiments the antibody molecule comprise a multispecific molecule, e.g., a bispecific molecule, e.g., as described herein.

In some embodiments, the heavy or light chain variable domain, or both, of, the anti-TCRβV antibody molecule includes an amino acid sequence, which is substantially identical to an amino acid disclosed herein, e.g., at least 80%, 85%, 90%, 92%, 95%, 97%, 98%, 99% or higher identical to a variable region of an antibody described herein, e.g., an antibody as described in Table 4 or Table 5, or encoded by the nucleotide sequence in Table 4 or Table 5; or which differs at least 1 or 5 residues, but less than 40, 30, 20, or 10 residues, from a variable region of an antibody described herein.

In some embodiments, the anti-TCRβV antibody molecule comprises at least one, two, three, or four antigen-binding regions, e.g., variable regions, having an amino acid sequence as set forth in Table 4 or Table 5, or a sequence substantially identical thereto (e.g., a sequence at least about 85%, 90%, 95%, 99% or more identical thereto, or which differs by no more than 1, 2, 5, 10, or 15 amino acid residues from the sequences shown in Table 4 or Table 5. In another embodiment, the anti-TCRβV antibody molecule includes a VH and/or VL domain encoded by a nucleic acid having a nucleotide sequence as set forth in Table 4 or Table 5, or a sequence substantially identical thereto (e.g., a sequence at least about 85%, 90%, 95%, 99% or more identical thereto, or which differs by no more than 3, 6, 15, 30, or 45 nucleotides from the sequences shown in Table 4 or Table 5.

In some embodiments, the anti-TCRβV antibody molecule is a full antibody or fragment thereof (e.g., a Fab, F(ab′)₂, Fv, or a single chain Fv fragment (scFv)). In embodiments, the anti-TCRβV antibody molecule antibody molecule is a monoclonal antibody or an antibody with single specificity. In some embodiments, the anti-TCRβV antibody molecule can also be a humanized, chimeric, camelid, shark, or an in vitro-generated antibody molecule. In some embodiments, the anti-TCRβV antibody molecule is a humanized antibody molecule. The heavy and light chains of the anti-TCRβV antibody molecule can be full-length (e.g., an antibody can include at least one, and preferably two, complete heavy chains, and at least one, and preferably two, complete light chains) or can include an antigen-binding fragment (e.g., a Fab, F(ab′)2, Fv, a single chain Fv fragment, a single domain antibody, a diabody (dAb), a bivalent antibody, or bispecific antibody or fragment thereof, a single domain variant thereof, or a camelid antibody).

In some embodiments, the anti-TCRβV antibody molecule is in the form of a multispecific molecule, e.g., a bispecific molecule, e.g., as described herein.

In some embodiments, the anti-TCRβV antibody molecule has a heavy chain constant region (Fc) chosen from, e.g., the heavy chain constant regions of IgG1, IgG2, IgG3, IgG4, IgM, IgA1, IgA2, IgD, and IgE. In some embodiments, the Fc region is chosen from the heavy chain constant regions of IgG1, IgG2, IgG3, and IgG4. In some embodiments, the Fc region is chosen from the heavy chain constant region of IgG1 or IgG2 (e.g., human IgG1, or IgG2). In some embodiments, the heavy chain constant region is human IgG1.

In some embodiments, the antibody comprises a heavy chain that shares at least 80%, 85%, 90%, 92%, 95%, 97%, 98%, 99%, or 100% identity to SEQ ID Nos: 83-87; the antibody comprises a light chain that shares at least 80%, 85%, 90%, 92%, 95%, 97%, 98%, 99%, or 100% identity to SEQ ID Nos: 88-95.

In some embodiments, the antibody comprises a heavy chain that shares at least 80%, 85%, 90%, 92%, 95%, 97%, 98%, 99%, or 100% identity to SEQ ID Nos: 108-111; the antibody comprises a light chain that shares at least 80%, 85%, 90%, 92%, 95%, 97%, 98%, 99%, or 100% identity to SEQ ID Nos: 112-114.

In some embodiments, the antibody comprises a heavy chain that shares at least 80%, 85%, 90%, 92%, 95%, 97%, 98%, 99%, or 100% identity to SEQ ID Nos: 127-131; the antibody comprises a light chain that shares at least 80%, 85%, 90%, 92%, 95%, 97%, 98%, 99%, or 100% identity to SEQ ID Nos: 132-136.

In some embodiments, the antibody comprises a heavy chain that shares at least 80%, 85%, 90%, 92%, 95%, 97%, 98%, 99%, or 100% identity to SEQ ID Nos: 149-153; the antibody comprises a light chain that shares at least 80%, 85%, 90%, 92%, 95%, 97%, 98%, 99%, or 100% identity to SEQ ID Nos: 154-158.

In some embodiments, the antibody comprises a heavy chain that shares at least 80%, 85%, 90%, 92%, 95%, 97%, 98%, 99%, or 100% identity to SEQ ID Nos: 170-174; the antibody comprises a light chain that shares at least 80%, 85%, 90%, 92%, 95%, 97%, 98%, 99%, or 100% identity to SEQ ID Nos: 175-181.

In some embodiments, the antibody comprises a heavy chain that shares at least 80%, 85%, 90%, 92%, 95%, 97%, 98%, 99%, or 100% identity to SEQ ID Nos: 194-199; the antibody comprises a light chain that shares at least 80%, 85%, 90%, 92%, 95%, 97%, 98%, 99%, or 100% identity to SEQ ID Nos: 200-204.

In some embodiments, the antibody comprises a heavy chain that shares at least 80%, 85%, 90%, 92%, 95%, 97%, 98%, 99%, or 100% identity to SEQ ID Nos: 217-221; the antibody comprises a light chain that shares at least 80%, 85%, 90%, 92%, 95%, 97%, 98%, 99%, or 100% identity to SEQ ID Nos: 222-227.

In some embodiments, the antibody comprises a heavy chain that shares at least 80%, 85%, 90%, 92%, 95%, 97%, 98%, 99%, or 100% identity to SEQ ID Nos: 240-243; the antibody comprises a light chain that shares at least 80%, 85%, 90%, 92%, 95%, 97%, 98%, 99%, or 100% identity to SEQ ID Nos: 244-249.

In some embodiments, the antibody comprises a heavy chain that shares at least 80%, 85%, 90%, 92%, 95%, 97%, 98%, 99%, or 100% identity to SEQ ID Nos: 262-265; the antibody comprises a light chain that shares at least 80%, 85%, 90%, 92%, 95%, 97%, 98%, 99%, or 100% identity to SEQ ID Nos: 266-269.

In some embodiments, the antibody comprises a heavy chain that shares at least 80%, 85%, 90%, 92%, 95%, 97%, 98%, 99%, or 100% identity to SEQ ID Nos: 282-284; the antibody comprises a light chain that shares at least 80%, 85%, 90%, 92%, 95%, 97%, 98%, 99%, or 100% identity to SEQ ID Nos: 285-288.

TABLE 4
Amino acid sequences for murine and humanized antibody molecules.
The antibody molecules include murine monoclonal antibody TM23
(also known as 4H11) and humanized monoclonal antibodies. The TM23
is also disclosed in U.S. Pat. No. 5,861,155, which is
incorporated by reference herein
TM23 (murine) (also referred to as 4H11 and BJM1162)
Binds to human TCRVβ 5-5, 5-6

| | | |
|---|---|---|
| SEQ ID NO: 70 | HC CDR1 (Kabat) | AYGVN |
| SEQ ID NO: 71 | HC CDR2 (Kabat) | MIWGDGNTDYNSALKS |
| SEQ ID NO: 72 | HC CDR3 (Kabat) | DRVTATLYAMDY |
| SEQ ID NO: 73 | HC CDR1 (Chothia) | GFSLTAY |
| SEQ ID NO: 74 | HC CDR2 (Chothia) | WGDGN |
| SEQ ID NO: 72 | HC CDR3 (Chothia) | DRVTATLYAMDY |
| SEQ ID NO: 75 | HC CDR1 (Combined) | GFSLTAYGVN |
| SEQ ID NO: 71 | HC CDR2 (Combined) | MIWGDGNTDYNSALKS |
| SEQ ID NO: 72 | HC CDR3 (Combined) | DRVTATLYAMDY |
| SEQ ID NO: 76 | LC CDR1 (Kabat) | SASQGISNYLN |
| SEQ ID NO: 77 | LC CDR2 (Kabat) | YTSSLHS |
| SEQ ID NO: 78 | LC CDR3 (Kabat) | QQYSKLPRT |
| SEQ ID NO: 76 | LC CDR1 (Chothia) | SASQGISNYLN |
| SEQ ID NO: 77 | LC CDR2 (Chothia) | YTSSLHS |
| SEQ ID NO: 78 | LC CDR3 (Chothia) | QQYSKLPRT |
| SEQ ID NO: 76 | LC CDR1 (Combined) | SASQGISNYLN |
| SEQ ID NO: 77 | LC CDR2 (Combined) | YTSSLHS |
| SEQ ID NO: 78 | LC CDR3 (Combined) | QQYSKLPRT |
| SEQ ID NO: 79 | VL | DIQMTQTTSSLSASLGDRVTISVSASQGISNYLNWYQQK |
| | | PDGTVKLLIYYTSSLHSGVPSRFSGSGSGTDYSLTISNL |
| | | EPEDIATYYCQQYSKLPRTFGGGTKVEIK |
| SEQ ID NO: 80 | VH | QVQLKESGPGLVAPSQSLSITCTBVSGFSLTAYGVNWVR |
| | | QPPGKGLEWLGMIWGDGNTDYNSALKSRLSISKDNSKSQ |
| | | VFLKMNSLQTDDTARYYCARDRVTATLYAMDYWGQGTSV |
| | | TVSS |

TM23 (humanized −1)
Binds to human TCRVβ 5-5, 5-6

| | | |
|---|---|---|
| SEQ ID NO: 70 | HC CDR1 (Kabat) | AYGVN |
| SEQ ID NO: 71 | HC CDR2 (Kabat) | MIWGDGNTDYNSALKS |
| SEQ ID NO: 72 | HC CDR3 (Kabat) | DRVTATLYAMDY |
| SEQ ID NO: 73 | HC CDR1 (Chothia) | GFSLTAY |
| SEQ ID NO: 74 | HC CDR2 (Chothia) | WGDGN |
| SEQ ID NO: 72 | HC CDR3 (Chothia) | DRVTATLYAMDY |
| SEQ ID NO: 75 | HC CDR1 (Combined) | GFSLTAYGVN |
| SEQ ID NO: 71 | HC CDR2 (Combined) | MIWGDGNTDYNSALKS |
| SEQ ID NO: 72 | HC CDR3 (Combined) | DRVTATLYAMDY |
| SEQ ID NO: 76 | LC CDR1 (Kabat) | SASQGISNYLN |
| SEQ ID NO: 77 | LC CDR2 (Kabat) | YTSSLHS |
| SEQ ID NO: 78 | LC CDR3 (Kabat) | QQYSKLPRT |
| SEQ ID NO: 76 | LC CDR1 (Chothia) | SASQGISNYLN |
| SEQ ID NO: 77 | LC CDR2 (Chothia) | YTSSLHS |
| SEQ ID NO: 78 | LC CDR3 (Chothia) | QQYSKLPRT |
| SEQ ID NO: 76 | LC CDR1 (Combined) | SASQGISNYLN |
| SEQ ID NO: 77 | LC CDR2 (Combined) | YTSSLHS |
| SEQ ID NO: 78 | LC CDR3 (Combined) | QQYSKLPRT |
| SEQ ID NO: 81 | VL | DIQMTQSPSSLSASVGDRVTITCSASQGISNYLNWYQQTP |
| | | GKAPKLLIYYTSSLHSGVPSRFSGSGSGTDYTFTISSLQP |
| | | EDIATYYCQQYSKLPRTFGQGTKLQIT |
| SEQ ID NO: 82 | VH | QVQLQESGPGLVRPSQTLSLTCTVSGFSLTAYGVNWVRQP |
| | | PGRGLEWLGMIWGDGNTDYNSALKSRVTMLKDTSKNQFSL |
| | | RLSSVTAADTAVYYCARDRVTATLYAMDYW GQGSLVTVS |
| | | S |

TM23 (humanized) VHs
Binds to human TCRVβ 5-5, 5-6

| | | |
|---|---|---|
| SEQ ID NO: 83 | VH-1 | QVTLKESGPVLVKPTETLTLTCTVSGFSLTAYGVNWVRQP |
| | | PGKALEWLGMIWGDGNTDYNSALKSRLTISKDNSKSQVVL |
| | | TMTNMDPVDTATYYCARDRVTATLYAMDYWGQGTLVTVSS |
| SEQ ID NO: 84 | VH-2 | QVTLKESGPALVKPTQTLTLTCTVSGFSLTAYGVNWVRQP |
| | | PGKALEWLGMIWGDGNTDYNSALKSRLTISKDNSKSQVVL |
| | | TMTNMDPVDTATYYCARDRVTATLYAMDYWGQGTLVTVSS |
| SEQ ID NO: 85 | VH-3 | QVQLQESGPGLVKPSGTLSLTCAVSGFSLTAYGVNWVRQP |
| | | PGKGLEWLGMIWGDGNTDYNSALKSRLTISKDNSKSQVSL |
| | | KLSSVTAADTAVYYCARDRVTATLYAMDYWGQGLTVTVSS |
| SEQ ID NO: 86 | VH-4 | EVQLVESGGGLVQPGPSLRLSCTVSGFSLTAYGVNWVRQA |
| | | PGKGLEWLGMIWGDGNTDYNSALKSRLTISKDNSKSIVYL |
| | | QMNSLKEDTAVYYCARDRVTATLYAMDYWGQGLTVSS |
| SEQ ID NO: 87 | VH-5 | QVLQQSGPGLVKPSGTSLTCAVSGFSLTAYGVNWVRQSPS |
| | | RGLEWLGMIWGDGNTDYNSALKSRLTINKDNSKSQVSLQL |
| | | NSVTPEDTAVYYCARDRVTATLYAMDYWGQGTLVTVSS |

TM23 (humanized) VLs
Binds to human TCRVβ 5-5, 5-6

| | | |
|---|---|---|
| SEQ ID NO: 88 | VL-1 | DIQMTQSPSFLSASVGDRVTITCSASQGISNYLNWYQQKP |
| | | GKAVKLLIYYTSSLHSGVPSRFSGSGSGTEYTLTISSLQP |
| | | EDFATYYCQQYSKLPRTFGGGTKVEIK |
| SEQ ID NO: 89 | VL-2 | DIQMTQSPSSLSASVGDRVTITCSASQGISNYLNWYQQKP |
| | | GKAVKLLIYYTSSLHSGVPSRFSGSGSGTDYTLTISSLQP |
| | | EDFATYYCQQYSKLPRTFGGGTKVEIK |
| SEQ ID NO: 90 | VL-3 | DIQMTQSPSSLSASVGDRVTITCSASQGISNYLNWYQQKP |
| | | GKVVKLLIYYTSSLHSGVPSRFSGSGSGTDYTLTISSLQP |
| | | EDVATYYCQQYSKLPRTFGGGTKVEIK |
| SEQ ID NO: 91 | VL-4 | AIRMTQSPFSLSASVGDRVTITCSASQGISNYLNWYQQKP |
| | | AKAVKLFIYYTSSLHSGVPSRFSGSGSGTDYTLTISSLQP |
| | | EDFATYYCQQYSKLPRTFGGGTKVEIK |
| SEQ ID NO: 92 | VL-5 | DIQMTQSPSAMSASVGDRVTITCSASQGISNYLNWYQQKP |
| | | GKVVKRLIYYTSSLHSGVPSRFSGSGSGTEYTLTISSLQP |
| | | EDFATYYCQQYSKLPRTFGGGTKVEIK |
| SEQ ID NO: 93 | VL-6 | EIVMTQSPPTLSLSPGERVTLSCSASQGISNYLNWYQQKP |
| | | GQAVKLLIYYTSSLHSGIPARFSGSGSGTDYTLTISSLQP |
| | | EDFAVYYCQQYSKLPRTFGGGTKVEIK |
| SEQ ID NO: 94 | VL-7 | DIVMTQTPLSLSVTPGQPASISCSASQGISNYLNWYLQKP |
| | | GQSVKLLIYYTSSLHSGVPDRFSGSGSGTDYTLKISRVEA |
| | | EDVGVYYCQQYSKLPRTFGGGTKVEIK |
| SEQ ID NO: 95 | VL-8 | DIVMTQSPAFLSVTPGEKVTITCSASQGISNYLNWYQQKP |
| | | DQAVKLLIYYTSSLHSGVPSRFSGSGSGTDYTFTISSLEA |
| | | EDAATYYCQQYSKLPRTFGGGTKVEIK |

TABLE 5
Amino acid sequences for murine and humanized antibody molecules. The
antibody molecules include murine and humanized antibodies that bind
human TCRβV. Antibodies disclosed in the table include, MPB2D5,
CAS1.1.3, IMMU222, REA1062, JOVI-3, S5111, MH3-2, 4H11. MPB2D5 binds
human TCRβV2 (TRβV 20-1). CAS1.1.3 binds human TCRβV14 (TRβV 27).
IMMU 222 binds human TCRβV13.1 (TRβV 6-5,6-6,6-9). REA1062 binds human
TCRβV5.1 (TRβV 5-1). JOVI-3 binds human TCRβV3.1 (TRβV 28). S511 binds
human TCRβV12 (TRβV 10-1,10-2,10-3). MH3 binds human TCRβV5
(TRβV 5-5, 5-6). 4H11 binds human TCRβV5 (TRβV 5-5,5-6).
MPB2D5 (murine), also referred to here as BJ1188, BJ1190 and REA654
Binds to human TCRVβ 20-1

| | | |
|---|---|---|
| SEQ ID NO: 96 | HC CDR1 (Kabat) | SAYMH |
| SEQ ID NO: 97 | HC CDR2 (Kabat) | RIDPATGKTKYAPKFQA |
| SEQ ID NO: 98 | HC CDR3 (Kabat) | SLNWDYGLDY |
| SEQ ID NO: 99 | HC CDR1 (Chothia) | GFNIKSA |
| SEQ ID NO: 100 | HC CDR2 (Chothia) | DPATGK |
| SEQ ID NO: 98 | HC CDR3 (Chothia) | SLNWDYGLDY |
| SEQ ID NO: 101 | HC CDR1 (Combined) | GFNIKSAYMH |
| SEQ ID NO: 97 | HC CDR2 (Combined)) | RIDPATGKTKYAPKFQA |
| SEQ ID NO: 98 | HC CDR3 (Combined) | SLNWDYGLDY |
| SEQ ID NO: 102 | LC CDR1 (Kabat) | RASKSVSILGTHLIH |
| SEQ ID NO: 103 | LC CDR2 (Kabat) | AASNLES |
| SEQ ID NO: 104 | LC CDR3 (Kabat) | QQSIEDPWT |
| SEQ ID NO: 105 | LC CDR1 (Chothia) | SKSVSILGTHL |
| SEQ ID NO: 103 | LC CDR2 (Chothia) | AASNLES |
| SEQ ID NO: 104 | LC CDR3 (Chothia) | QQSIEDPWT |
| SEQ ID NO: 102 | LC CDR1 (Combined) | RASKSVSILGTHLIH |
| SEQ ID NO: 103 | LC CDR2 (Combined) | AASNLES |
| SEQ ID NO: 104 | LC CDR3 (Combined) | QQSIEDPWT |
| SEQ ID NO: 106 | VL | DIVLTQSPASLAVSLGQRATISCRASKSVSILGTHLIH |
| | | WYQQKPGQPPKLLIYAASNLESGVPARFSGSGSETV |
| | | FTLNIHPVEEEDAATYFCQQSIEDPWTFGGGTKLGIK |
| SEQ ID NO: 107 | VH | EVQLQQSVADLVRPGASLKLSCTASGFNIKSAYMH |
| | | WVIQRPDQGPECLGRIDPATGKTKYAPKFQAKATIT |
| | | ADTSSNTAYLQLSSLTSEDTAIYYCTRSLNWDYGLD |
| | | YWGQGTSVTVSS |

MPB2D5 (humanized) VHs
Binds to human TCRVβ 20-1

| | | |
|---|---|---|
| SEQ ID NO: 108 | VH-1 | QVQLVQSGAEVKKPGASVKVSCKASGFNIKSAYMH |
| | | WVRQAPGQGLEWMGRIDPATGKTKYAPKFQARVT |
| | | MTADTSTNTAYMELSSLRSEDTAVYYCARSLNWDY |
| | | GLDYWGQGTLVTVSS |
| SEQ ID NO: 109 | VH-2 | QVQLVQSGAEVKKPGASVKVSCKASGFNIKSAYMH |
| | | WVRQAPGQEPGCMGRIDPATGKTKYAPKFQARVT |
| | | MTADTSINTAYTELSSLRSEDTATYYCARSLNWDYG |
| | | LDYWGQGTLVTVSS |
| SEQ ID NO: 110 | VH-3 | QVQLVQSGAEVKKPGSSVKVSCKASGFNIKSAYMH |
| | | WVRQAPGQGLEWMGRIDPATGKTKYAPKFQARVTI |
| | | TADTSTNTAYMELSSLRSEDTAVYYCARSLNWDYG |
| | | LDYWGQGTLVTVSS |
| SEQ ID NO: 111 | VH-4 | QVQLVQSGAEVKKPGASVKVSCKASGFNIKSAYMH |
| | | WVRQAPGQRLEWMGRIDPATGKTKYAPKFQARVTI |
| | | TADTSANTAYMELSSLRSEDTAVYYCARSLNWDYG |
| | | LDYWGQGTLVTVSS |

MPB2D5 (humanized) VLs
Binds to human TCRVβ 20-1

| | | |
|---|---|---|
| SEQ ID NO: 112 | VL-1 | EIVLTQSPATLSLSPGERATLSCRASKSVSILGTHLIH |
| | | WYQQKPGQAPRLLIYAASNLESGIPARFSGSGSETDF |
| | | TLTISSLEPEDFAVYFCQQSIEDPFGGGTKVEIK |
| SEQ ID NO: 113 | VL-2 | EIVLTQSPATLSLSPGERATLSCRASKSVSILGTHLIH |
| | | WYQQKPGLAPRLLIYAASNLESGIPDRFSGSGSETDF |
| | | TLTISRLEPEDFAVYFCQQSIEDPFGGGTKVEIK |
| SEQ ID NO: 114 | VL-3 | EIVLTQSPGTLSLSPGERATLSCRASKSVSILGTHLIH |
| | | WYQQKPGQAPRLLIYAASNLESGIPDRFSGSGSETDF |
| | | TLTISRLEPEDFAVYFCQQSIEDPFGGGTKVEIK |

CAS1.1.3 (murine) also referred to herein as BJ1460
Binds to human TCRVβ 27

| | | |
|---|---|---|
| SEQ ID NO: 115 | HC CDR1 (Kabat) | DTYMY |
| SEQ ID NO: 116 | HC CDR2 (Kabat) | RIDPANGNTKYDPKFQD |
| SEQ ID NO: 117 | HC CDR3 (Kabat) | GSYYYAMDY |
| SEQ ID NO: 118 | HC CDR1 (Chothia) | GFKTEDT |
| SEQ ID NO: 119 | HC CDR2 (Chothia) | DPANGN |
| SEQ ID NO: 117 | HC CDR3 (Chothia) | GSYYYAMDY |
| SEQ ID NO: 120 | HC CDR1 (Combined) | GFKTEDTYMY |
| SEQ ID NO: 116 | HC CDR2 (Combined)) | RIDPANGNTKYDPKFQD |
| SEQ ID NO: 117 | HC CDR3 (Combined) | GSYYYAMDY |
| SEQ ID NO: 121 | LC CDR1 (Kabat) | RASESVDSYGNSFMEI |
| SEQ ID NO: 122 | LC CDR2 (Kabat) | RASNLES |
| SEQ ID NO: 123 | LC CDR3 (Kabat) | QQSNEDPYT |
| SEQ ID NO: 124 | LC CDR1 (Chothia) | SESVDSYGNSF |
| SEQ ID NO: 122 | LC CDR2 (Chothia) | RASNLES |
| SEQ ID NO: 123 | LC CDR3 (Chothia) | QQSNEDPYT |
| SEQ ID NO: 121 | LC CDR1 (Combined) | RASESVDSYGNSFMEI |
| SEQ ID NO: 122 | LC CDR2 (Combined) | RASNLES |
| SEQ ID NO: 123 | LC CDR3 (Combined) | QQSNEDPYT |
| SEQ ID NO: 125 | VL | DIVLTQSPASLAVSLGQRATISCRASESVDSYGNSFM |
| | | HWYQQKPGQPPKLLIYRASNLESGIPARFSGSGSRTD |
| | | FTLTINPVEADDVATYYCQQSNEDPYTFGGGTKLEI |
| | | K |
| SEQ ID NO: 126 | VH | EVQLQQSGAELVKPGASVKLSCTASGFKTEDTYMY |
| | | WVKQRPEQGLEWIGRIDPANGNTKYDPKFQDKATI |
| | | TADSSSNTAYLQLSSLPSEDTAVYYCARGSYYYAM |
| | | DYWGQGTSVTVSS |

CAS1.1.3 (humanized) VHs
Binds to human TCRVβ 27

| | | |
|---|---|---|
| SEQ ID NO: 127 | VH-1 | QVQLVQSGAEVKKPGSSVKVSCKASGFKTEDTYMY |
| | | WVRQAPGQGLEWIGRIDPANGNTKYDPKFQDRATI |
| | | TADSSTNTAYMELSSLRSEDTAVYYCARGSYYYAM |
| | | DYWGQGTLVTVSS |
| SEQ ID NO: 128 | VH-2 | QVQLVQSGAEVKKPGASVKVSCKASGFKTEDTYM |
| | | YWVRQAPGQRLEWIGRIDPANGNTKYDPKFQDRAT |
| | | ITADSSANTAYMELSSLRSEDTAVYYCARGSYYYA |
| | | MDYWGQGTLVTVSS |
| SEQ ID NO: 129 | VH-3 | EVQLVESGGGLVQPGGSLKLSCAASGFKTEDTYMY |
| | | WVRQASGKGLEWIGRIDPANGNTKYDPKFQDRATI |
| | | SADSSKNTAYLQMNSLKTEDTAVYYCARGSYYYA |
| | | MDYWGQGTLVTVSS |
| SEQ ID NO: 130 | VH-4 | EVQLVQSGAEVKKPGESLRISCKASGFKTEDTYMY |
| | | WVRQMPGKGLEWIGRIDPANGNTKYDPKFQDQATI |
| | | SADSSINTAYLQWSSLKASDTAMYYCARGSYYYAM |
| | | DYWGQGTLVTVSS |
| SEQ ID NO: 131 | VH-5 | QVQLVQSGSELKKPGASVKVSCKASGFKTEDTYMY |
| | | WVRQAPGQGLEWIGRIDPANGNTKYDPKFQDRAVI |
| | | SADSSVNTAYLQISSLKAEDTAVYYCARGSYYYAM |
| | | DYWGQGTLVTVSS |

CAS1.1.3 (humanized) VLs
Binds to human TCRVβ 27

| | | |
|---|---|---|
| SEQ ID NO: 132 | VL-1 | DIVLTQSPDSLAVSLGERATINCRASESVDSYGNSFM |
| | | HWYQQKPGQPPKLLIYRASNLESGVPDRFSGSGSRT |
| | | DFTLTISSLQAEDVAVYYCQQSNEDPYTFGQGTKLEI |
| | | K |
| SEQ ID NO: 133 | VL-2 | EIVLTQSPATLSLSPGERATLSCRASESVDSYGNSFM |
| | | HWYQQKPGQAPKLLIYRASNLESGIPARFSGSGSRT |
| | | DFTLTISRLEPEDFAVYYCQQSNEDPYTFGQGTKLEI |
| | | K |
| SEQ ID NO: 134 | VL-3 | DIQLTQSPSSLSASVGDRVTITCRASESVDSYGNSFM |
| | | HWYQQKPGQAPKLLIYRASNLESGVPSRFSGSGSRT |
| | | DFTLTISSLQPEDVATYYCQQSNEDPYTFGQGTKLEI |
| | | K |
| SEQ ID NO: 135 | VL-4 | AIQLTQSPSSLSASVGDRVTITCRASESVDSYGNSFM |
| | | HWYQQKPGKAPKLLIYRASNLESGVPSRFSGSGSRT |
| | | DFTLTISSLQPEDFATYYCQQSNEDPYTFGQGTKLEI |
| | | K |
| SEQ ID NO: 136 | VL-5 | EIVLTQSPDFQSVTPKEKVTITCRASESVDSYGNSFM |
| | | HWYQQKPDQSPKLLIYRASNLESGVPSRFSGSGSRT |
| | | DFTLTINSLEAEDAATYYCQQSNEDPYTFGQGTKLEI |
| | | K |

IMMU222 (murine) also referred to as BJ1461
Binds to human TCRVβ 6-5,6-6,6-9

| | | |
|---|---|---|
| SEQ ID NO: 137 | HC CDR1 (Kabat) | SYAMS |
| SEQ ID NO: 138 | HC CDR2 (Kabat) | HISNGGDYIYYADTVKG |
| SEQ ID NO: 139 | HC CDR3 (Kabat) | PSYYSDPWFFDV |
| SEQ ID NO: 140 | HC CDR1 (Chothia) | GFTFRSY |
| SEQ ID NO: 141 | HC CDR2 (Chothia) | SNGGDY |
| SEQ ID NO: 139 | HC CDR3 (Chothia) | PSYYSDPWFFDV |
| SEQ ID NO: 142 | HC CDR1 (Combined) | GFTFRSYAMS |
| SEQ ID NO: 138 | HC CDR2 (Combined)) | HISNGGDYIYYADTVKG |
| SEQ ID NO: 139 | HC CDR3 (Combined) | PSYYSDPWFFDV |
| SEQ ID NO: 143 | LC CDR1 (Kabat) | SAGSSVSFMH |
| SEQ ID NO: 144 | LC CDR2 (Kabat) | DTSKLAS |
| SEQ ID NO: 145 | LC CDR3 (Kabat) | LQGSGFPLT |
| SEQ ID NO: 146 | LC CDR1 (Chothia) | GSSVSF |
| SEQ ID NO: 144 | LC CDR2 (Chothia) | DTSKLAS |
| SEQ ID NO: 145 | LC CDR3 (Chothia) | LQGSGFPLT |
| SEQ ID NO: 143 | LC CDR1 (Combined) | SAGSSVSFMH |
| SEQ ID NO: 144 | LC CDR2 (Combined) | DTSKLAS |
| SEQ ID NO: 145 | LC CDR3 (Combined) | LQGSGFPLT |
| SEQ ID NO: 147 | VL | ENVLTQSPAIMSASPGEKVTMTCSAGSSVSFMEIWY |
| | | QQKSSTSPKLWIYDTSKLASGVPGRFSGSGSGNSFSL |
| | | TISMEAEDVAIYYCLQGSGFPLTFGSGTKLEIK |
| SEQ ID NO: 148 | VH | DVKLVESGEGLVKPGGSLKLSCAASGFTFRSYAMS |
| | | WVRQTPEKRLEWVAHISNGGDYIYYADTVKGRFTIS |
| | | RDNARNTLYLQMSSLKSEDTAMYYCTRPSYYSDPW |
| | | FFDVWGTGTTVTVSS |

IMMU222 (humanized) VHs
Binds to human TCRVβ 6-5,6-6,6-9

| | | |
|---|---|---|
| SEQ ID NO: 149 | VH-1 | EVQLVESGGGLVQPGGSLRLSCAASGFTFRSYAMS |
| | | WVRQAPGKGLEWVAHISNGGDYIYYADTVKGRFTI |
| | | SRDNAKNSLYLQMNSLRAEDTAVYYCTRPSYYSDP |
| | | WFFDVWGQGTTVTVSS |
| SEQ ID NO: 150 | VH-2 | QVQLVESGGGVVQPGRSLRLSCAASGFTFRSYAMS |
| | | WVRQAPGKGLEWVAHISNGGDYIYYADTVKGRFTI |
| | | SRDNSKNTLYLQMSSLRAEDTAVYYCTRPSYYSDP |
| | | WFFDVWGQGTTVTVSS |
| SEQ ID NO: 151 | VH-3 | EVQLVESGGGLVQPGGSLRLSCAASGFTFRSYAMS |
| | | WVRQAPGKGLEWVAHISNGGDYIYYADTVKGRFTI |
| | | SRDNSKNTLYLQMNSLRAEDTAVYYCTRPSYYSDP |
| | | WFFDVWGQGTTVTVSS |
| SEQ ID NO: 152 | VH-4 | QVQLVQSGSELKKPGASVKVSCKASGFTFRSYAMS |
| | | WVRQAPGQGLEWVAHISNGGDYIYYADTVKGRFVI |
| | | SRDNSVNTLYLQISSLKAEDTAVYYCTRPSYYSDPW |
| | | FFDVWGQGTTVTVSS |
| SEQ ID NO: 153 | VH-5 | QVQLVQSGAEVKKPGASVKVSCKASGFTFRSYAMS |
| | | WVRQAPGQRLEWVAHISNGGDYIYYADTVKGRFTI |
| | | TRDNSANTLYMELSSLRSEDTAVYYCTRPSYYSDP |
| | | WFFDVWGQGTTVTVSS |

IMMU222 (humanized) VLs
Binds to human TCRVβ 6-5,6-6,6-9

| | | |
|---|---|---|
| SEQ ID NO: 154 | VL-1 | ENVLTQSPATLSLSPGERATLSCSAGSSVSFMHWYQ |
| | | QKPGQAPKLLIYDTSKLASGIPARFSGSGSGNDFTLTI |
| | | SSLEPEDFAVYYCLQGSGFPLTFGQGTKLEIK |
| SEQ ID NO: 155 | VL-2 | ENVLTQSPDFQSVTPKEKVTITCSAGSSVSFMEIWYQ |
| | | QKPDQSPKLLIYDTSKLASGVPSRFSGSGSGNDFTLT |
| | | INSLEAEDAATYYCLQGSGFPLTFGQGTKLEIK |
| SEQ ID NO: 156 | VL-3 | DNQLTQSPSSLSASVGDRVTITCSAGSSVSFMEIWYQ |
| | | QKPGKVPKLLIYDTSKLASGVPSRFSGSGSGNDFTLT |
| | | ISSLQPEDVATYYCLQGSGFPLTFGQGTKLEIK |
| SEQ ID NO: 157 | VL-4 | ANQLTQSPSSLSASVGDRVTITCSAGSSVSFMEIWYQ |
| | | QKPGKAPKLLIYDTSKLASGVPSRFSGSGSGNDFTLT |
| | | ISSLQPEDFATYYCLQGSGFPLTFGQGTKLEIK |
| SEQ ID NO: 158 | VL-5 | DNVLTQSPDSLAVSLGERATINCSAGSSVSFMHWYQ |
| | | QKPGQPPKLLIYDTSKLASGVPDRFSGSGSGNDFTLT |
| | | ISSLQAEDVAVYYCLQGSGFPLTFGQGTKLEIK |

REA1062 (murine), also referred to as BJ1189
Binds to human TCRVβ 5-1

| | | |
|---|---|---|
| SEQ ID NO: 159 | HC CDR1 (Kabat) | DYNIH |
| SEQ ID NO: 160 | HC CDR2 (Kabat) | YINPYNGRTGYNQKFKA |
| SEQ ID NO: 161 | HC CDR3 (Kabat) | WDGSSYFDY |
| SEQ ID NO: 162 | HC CDR1 (Chothia) | GYTFTDYNIH |
| SEQ ID NO: 163 | HC CDR2 (Chothia) | NPYNGR |
| SEQ ID NO: 161 | HC CDR3 (Chothia) | WDGSSYFDY |
| SEQ ID NO: 162 | HC CDR1 (Combined) | GYTFTDYNIH |
| SEQ ID NO: 160 | HC CDR2 (Combined)) | YINPYNGRTGYNQKFKA |
| SEQ ID NO: 161 | HC CDR3 (Combined) | WDGSSYFDY |
| SEQ ID NO: 164 | LC CDR1 (Kabat) | SASSSVSYMH |
| SEQ ID NO: 165 | LC CDR2 (Kabat) | EISKLAS |
| SEQ ID NO: 166 | LC CDR3 (Kabat) | QQWNYPLLT |
| SEQ ID NO: 167 | LC CDR1 (Chothia) | SSSVSY |
| SEQ ID NO: 165 | LC CDR2 (Chothia) | EISKLAS |
| SEQ ID NO: 166 | LC CDR3 (Chothia) | QQWNYPLLT |
| SEQ ID NO: 164 | LC CDR1 (Combined) | SASSSVSYMH |
| SEQ ID NO: 165 | LC CDR2 (Combined) | EISKLAS |
| SEQ ID NO: 166 | LC CDR3 (Combined) | QQWNYPLLT |
| SEQ ID NO: 168 | VL | EIVLTQSPAITAASLGQKVTITCSASSSVSYMHWYQQ |
| | | KSGTSPKPWIYEISKLASGVPARFSGSGSGTSYSLTIS |
| | | SMEAEDAAIYYCQQWNYPLLTFGAGTKLELK |
| SEQ ID NO: 169 | VH | EVQLQQSGPVLVKPGASVRMSCKASGYTFTDYNIH |
| | | WVKQSHGRSLEWVGYINPYNGRTGYNQKFKAKAT |
| | | LTVDKSSSTAYMDLRSLTSEDSAVYYCARWDGSSY |
| | | FDYWGQGTTLTVSS |

REA1062 (humanized) VHs
Binds to human TCRVβ 5-1

| | | |
|---|---|---|
| SEQ ID NO: 170 | VH-1 | QVQLVQSGAEVKKPGSSVKVSCKASGYTFTDYNIH |
| | | WVRQAPGQGLEWVGYINPYNGRTGYNQKFKARAT |
| | | LTVDKSTSTAYMELSSLRSEDTAVYYCARWDGSSY |
| | | FDYWGQGTTVTVSS |
| SEQ ID NO: 171 | VH-2 | QVQLVQSGAEVKKPGASVKVSCKASGYTFTDYNIEI |
| | | WVRQAPGQGLEWVGYINPYNGRTGYNQKFKARAT |
| | | LTVDKSTSTAYMELRSLRSDDMAVYYCARWDGSS |
| | | YFDYWGQGTTVTVSS |
| SEQ ID NO: 172 | VH-3 | QVQLVQSGAEVKKPGASVKVSCKASGYTFTDYNIEI |
| | | WVRQATGQGLEWVGYINPYNGRTGYNQKFKARAT |
| | | LTVNKSISTAYMELSSLRSEDTAVYYCARWDGSSYF |
| | | DYWGQGTTVTVSS |
| SEQ ID NO: 173 | VH-4 | EVQLVESGGGLVQPGRSLRLSCTASGYTFTDYNIHW |
| | | VRQAPGKGLEWVGYINPYNGRTGYNQKFKARATLS |
| | | VDKSKSIAYLQMNSLKTEDTAVYYCARWDGSSYFD |
| | | YWGQGTTVTVSS |
| SEQ ID NO: 174 | VH-5 | QVQLVQSGSELKKPGASVKVSCKASGYTFTDYNIEI |
| | | WVRQAPGQGLEWVGYINPYNGRTGYNQKFKARAV |
| | | LSVDKSVSTAYLQISSLKAEDTAVYYCARWDGSSYF |
| | | DYWGQGTTVTVSS |

REA1062 (humanized) VLs
Binds to human TCRVβ 5-1

| | | |
|---|---|---|
| SEQ ID NO: 175 | VL-1 | EIVLTQSPATLSLSPGERATLSCSASSSVSYMHWYQQ |
| | | KPGQAPKLLIYEISKLASGIPARFSGSGSGTDYTLTIS |
| | | SLEPEDFAVYYCQQWNYPLLTFGQGTKLEIK |
| SEQ ID NO: 176 | VL-2 | EIVLTQSPATLSLSPGERATLSCSASSSVSYMHWYQQ |
| | | KPGQAPKLLIYEISKLASGIPARFSGSGSGTDYTLTIS |
| | | RLEPEDFAVYYCQQWNYPLLTFGQGTKLEIK |
| SEQ ID NO: 177 | VL-3 | EIVLTQSPDFQSVTPKEKVTITCSASSSVSYMEIWYQ |
| | | QKPDQSPKLLIYEISKLASGVPSRFSGSGSGTDYTLTI |
| | | NSLEAEDAATYYCQQWNYPLLTFGQGTKLEIK |
| SEQ ID NO: 178 | VL-4 | DIQLTQSPSFLSASVGDRVTITCSASSSVSYMEIWYQ |
| | | QKPGKAPKLLIYEISKLASGVPSRFSGSGSGTEYTLTI |
| | | SSLQPEDFATYYCQQWNYPLLTFGQGTKLEIK |
| SEQ ID NO: 179 | VL-5 | AIQLTQSPSSLSASVGDRVTITCSASSSVSYMEIWYQ |
| | | QKPGKAPKLLIYEISKLASGVPSRFSGSGSGTDYTLTI |
| | | SSLQPEDFATYYCQQWNYPLLTFGQGTKLEIK |
| SEQ ID NO: 180 | VL-6 | AIRLTQSPFSLSASVGDRVTITCSASSSVSYMEIWYQQ |
| | | KPAKAPKLFIYEISKLASGVPSRFSGSGSGTDYTLTIS |
| | | SLQPEDFATYYCQQWNYPLLTFGQGTKLEIK |
| SEQ ID NO: 181 | VL-7 | DIVLTQSPDSLAVSLGERATINCSASSSVSYMEIWYQ |
| | | QKPGQPPKLLIYEISKLASGVPDRFSGSGSGTDYTLTI |
| | | SSLQAEDVAVYYCQQWNYPLLTFGQGTKLEIK |

JOVI-3 (murine), also referred to as BJ1187
Binds to human TCRVβ 28

| | | |
|---|---|---|
| SEQ ID NO: 182 | HC CDR1 (Kabat) | GSWMN |
| SEQ ID NO: 183 | HC CDR2 (Kabat) | RIYPGDGDTDYSGKFKG |
| SEQ ID NO: 184 | HC CDR3 (Kabat) | SGYFNYVPVFDY |
| SEQ ID NO: 185 | HC CDR1 (Chothia) | GYTFSGS |
| SEQ ID NO: 186 | HC CDR2 (Chothia) | YPGDGD |
| SEQ ID NO: 184 | HC CDR3 (Chothia) | SGYFNYVPVFDY |
| SEQ ID NO: 187 | HC CDR1 (Combined) | GYTFSGSWMN |
| SEQ ID NO: 183 | HC CDR2 (Combined)) | RIYPGDGDTDYSGKFKG |
| SEQ ID NO: 184 | HC CDR3 (Combined) | SGYFNYVPVFDY |
| SEQ ID NO: 188 | LC CDR1 (Kabat) | SANSTVGYIE |
| SEQ ID NO: 189 | LC CDR2 (Kabat) | TTSNLAS |
| SEQ ID NO: 190 | LC CDR3 (Kabat) | HQWSFYPT |
| SEQ ID NO: 191 | LC CDR1 (Chothia) | NSTVGY |
| SEQ ID NO: 189 | LC CDR2 (Chothia) | TTSNLAS |
| SEQ ID NO: 190 | LC CDR3 (Chothia) | HQWSFYPT |
| SEQ ID NO: 188 | LC CDR1 (Combined) | SANSTVGYIE |
| SEQ ID NO: 189 | LC CDR2 (Combined) | TTSNLAS |
| SEQ ID NO: 190 | LC CDR3 (Combined) | HQWSFYPT |
| SEQ ID NO: 192 | VL | QIVLTQSPAIMSASLGEEIALTCSANSTVGYIEIWYQQ |
| | | KSGTSPKLLIYTTSNLASGVPSRFSGSGSGTFYSLTIS |
| | | SVEAEDAADYFCHQWSFYPTFGGGTKLEIK |
| SEQ ID NO: 193 | VH | QIQLQQSGPEVVKPGASVQISCKASGYTFSGSWMN |
| | | WVKQRPGKGLEWIGRIYPGDGDTDYSGKFKGRATL |
| | | TADKSSSTAYMRLSSLTSEDSAVYFCARSGYFNYVP |
| | | VFDYWGQGTTLSVSS |

JOVI-3 (humanized) VHs
Binds to human TCRVβ 28

| | | |
|---|---|---|
| SEQ ID NO: 194 | VH-1 | QIQLVQSGAEVKKPGASVKVSCKASGYTFSGSWMN |
| | | WVRQAPGQGLEWIGRIYPGDGDTDYSGKFKGRATL |
| | | TADKSTSTAYMELSSLRSEDTAVYYCARSGYFNYVP |
| | | VFDYWGQGTTVTVSS |
| SEQ ID NO: 195 | VH-2 | QIQLVQSGAEVKKPGSSVKVSCKASGYTFSGSWMN |
| | | WVRQAPGQGLEWIGRIYPGDGDTDYSGKFKGRATL |
| | | TADKSTSTAYMELSSLRSEDTAVYYCARSGYFNYVP |
| | | VFDYWGQGTTVTVSS |
| SEQ ID NO: 196 | VH-3 | EIQLVQSGAEVKKPGESLKISCKASGYTFSGSWMNW |
| | | VRQMPGKGLEWIGRIYPGDGDTDYSGKFKGQATLS |
| | | ADKSISTAYLQWSSLKASDTAMYYCARSGYFNYVP |
| | | VFDYWGQGTTVTVSS |
| SEQ ID NO: 197 | VH-4 | QIQLVQSGSELKKPGASVKVSCKASGYTFSGSWMN |
| | | WVRQAPGQGLEWIGRIYPGDGDTDYSGKFKGRAVL |
| | | SADKSVSTAYLQISSLKAEDTAVYYCARSGYFNYVP |
| | | VFDYWGQGTTVTVSS |
| SEQ ID NO: 198 | VH-5 | QIQLVQSGSELKKPGASVKVSCKASGYTFSGSWMN |
| | | WVRQAPGQGLEWIGRIYPGDGDTDYSGKFKGRAVL |
| | | SADKSVSMAYLQISSLKAEDTAVYYCARSGYFNYV |
| | | PVFDYWGQGTTVTVSS |
| SEQ ID NO: 199 | VH-6 | EIQLVESGGGLVQPGRSLRLSCTASGYTFSGSWMN |
| | | WVRQAPGKGLEWIGRIYPGDGDTDYSGKFKGRATL |
| | | SADKSKSIAYLQMNSLKTEDTAVYYCARSGYFNYV |
| | | PVFDYWGQGTTVTVSS |

JOVI-3 (humanized) VLs
Binds to human TCRVβ 28

| | | |
|---|---|---|
| SEQ ID NO: 200 | VL-1 | EIVLTQSPATLSLSPGERATLSCSANSTVGYIEIWYQQ |
| | | KPGQAPKLLIYTTSNLASGIPARFSGSGSGTDYTLTIS |
| | | SLEPEDFAVYFCHQWSFYPTFGQGTKLEIK |
| SEQ ID NO: 201 | VL-2 | DIQLTQSPSFLSASVGDRVTITCSANSTVGYIEIWYQQ |
| | | KPGKAPKLLIYTTSNLASGVPSRFSGSGSGTEYTLTIS |
| | | SLQPEDFATYFCHQWSFYPTFGQGTKLEIK |
| SEQ ID NO: 202 | VL-3 | EIVLTQSPATLSLSPGERATLSCSANSTVGYIEIWYQQ |
| | | KPGQAPKLLIYTTSNLASGIPARFSGSGPGTDYTLTIS |
| | | SLEPEDFAVYFCHQWSFYPTFGQGTKLEIK |
| SEQ ID NO: 203 | VL-4 | DIVLTQSPDSLAVSLGERATINCSANSTVGYIEIWYQ |
| | | QKPGQPPKLLIYTTSNLASGVPDRFSGSGSGTDYTLT |
| | | ISSLQAEDVAVYFCHQWSFYPTFGQGTKLEIK |
| SEQ ID NO: 204 | VL-5 | EIVLTQSPDFQSVTPKEKVTITCSANSTVGYIHWYQQ |
| | | KPDQSPKLLIYTTSNLASGVPSRFSGSGSGTDYTLTIN |
| | | SLEAEDAATYFCHQWSFYPTFGQGTKLEIK |

S511 (murine) Also referred to herein as TCRvb10
Binds to human TCRVβ 10-1,10-2,10-3

| | | |
|---|---|---|
| SEQ ID NO: 205 | HC CDR1 (Kabat) | SYGMS |
| SEQ ID NO: 206 | HC CDR2 (Kabat) | LISSGGSYTYYTDSVKG |
| SEQ ID NO: 207 | HC CDR3 (Kabat) | HGGNFFDY |
| SEQ ID NO: 208 | HC CDR1 (Chothia) | GFTFRSY |
| SEQ ID NO: 209 | HC CDR2 (Chothia) | SSGGSY |
| SEQ ID NO: 207 | HC CDR3 (Chothia) | HGGNFFDY |
| SEQ ID NO: 210 | HC CDR1 (Combined) | GFTFRSYGMS |
| SEQ ID NO: 206 | HC CDR2 (Combined)) | LISSGGSYTYYTDSVKG |
| SEQ ID NO: 207 | HC CDR3 (Combined) | HGGNFFDY |
| SEQ ID NO: 211 | LC CDR1 (Kabat) | SVSSSVSYMH |
| SEQ ID NO: 212 | LC CDR2 (Kabat) | DTSKLAS |
| SEQ ID NO: 213 | LC CDR3 (Kabat) | QQWSSNPQYT |
| SEQ ID NO: 214 | LC CDR1 (Chothia) | SSSVSY |
| SEQ ID NO: 212 | LC CDR2 (Chothia) | DTSKLAS |
| SEQ ID NO: 213 | LC CDR3 (Chothia) | QQWSSNPQYT |
| SEQ ID NO: 211 | LC CDR1 (Combined) | SVSSSVSYMH |
| SEQ ID NO: 212 | LC CDR2 (Combined) | DTSKLAS |
| SEQ ID NO: 213 | LC CDR3 (Combined) | QQWSSNPQYT |
| SEQ ID NO: 215 | VL | QIVLTQSPSIMSASPGEKVTMTCSVSSSVSYMEIWYQ |
| | | QKSGTSPKRWIYDTSKLASGVPARFSGSGSGTSYSLT |
| | | ISSMEAEDAATYYCQQWSSNPQYTFGGGTKLEIK |
| SEQ ID NO: 216 | VH | EVQLVESGGDLVKPGGSLKLSCAVSGFTFRSYGMS |
| | | WVRQTPDKRLEWVALISSGGSYTYYTDSVKGRFTIS |
| | | RDNAKNTLYLQMSSLKSEDTAIYYCSRHGGNFFDY |
| | | WGQGTTLTVSS |

S511 (humanized) VHs
Binds to human TCRVβ 10-1,10-2,10-3

| | | |
|---|---|---|
| SEQ ID NO: 217 | VH-1 | EVQLVESGGGLVKPGGSLRLSCAVSGFTFRSYGMS |
| | | WVRQAPGKGLEWVALISSGGSYTYYTDSVKGRFTIS |
| | | RDNSKNTLYLQMNSLKTEDTAVYYCSRHGGNFFDY |
| | | WGQGTTVTVSS |
| SEQ ID NO: 218 | VH-2 | EVQLVESGGGLVQPGGSLRLSCAVSGFTFRSYGMS |
| | | WVRQAPGKGLEWVALISSGGSYTYYTDSVKGRFTIS |
| | | RDNAKNSLYLQMNSLRAEDTAVYYCSRHGGNFFD |
| | | YWGQGTTVTVSS |
| SEQ ID NO: 219 | VH-3 | QVQLQESGPGLVKPSGTLSLTCAVSGFTFRSYGMSW |
| | | VRQPPGKGLEWVALISSGGSYTYYTDSVKGRFTISR |
| | | DNSKNQLSLKLSSVTAADTAVYYCSRHGGNFFDYW |
| | | GQGTTVTVSS |
| SEQ ID NO: 220 | VH-4 | QVQLVQSGAEVKKPGASVKVSCKVSGFTFRSYGMS |
| | | WVRQAPGQRLEWVALISSGGSYTYYTDSVKGRFTIT |
| | | RDNSANTLYMELSSLRSEDTAVYYCSRHGGNFFDY |
| | | WGQGTTVTVSS |
| SEQ ID NO: 221 | VH-5 | EVQLVQSGAEVKKPGESLKISCKVSGFTFRSYGMSW |
| | | VRQMPGKGLEWVALISSGGSYTYYTDSVKGQFTISR |
| | | DNPINTLYLQWSSLKASDTAMYYCSRHGGNFFDYW |
| | | GQGTTVTVSS |

SH11 (humanized) VLs
Binds to human TCRVβ 10-1,10-2,10-3

| | | |
|---|---|---|
| SEQ ID NO: 222 | VL-1 | AIQLTQSPSSLSASVGDRVTITCSVSSSVSYMEIWYQ |
| | | QKPGKAPKLLIYDTSKLASGVPSRFSGSGSGTDYTL |
| | | TISSLQPEDFATYYCQQWSSNPQYTFGQGTKLEIK |
| SEQ ID NO: 223 | VL-2 | DIQLTQSPSFLSASVGDRVTITCSVSSSVSYMEIWYQ |
| | | QKPGKAPKLLIYDTSKLASGVPSRFSGSGSGTEYTLT |
| | | ISSLQPEDFATYYCQQWSSNPQYTFGQGTKLEIK |
| SEQ ID NO: 224 | VL-3 | EIVLTQSPDFQSVTPKEKVTITCSVSSSVSYMEIWYQ |
| | | QKPDQSPKLLIYDTSKLASGVPSRFSGSGSGTDYTLT |
| | | INSLEAEDAATYYCQQWSSNPQYTFGQGTKLEIK |
| SEQ ID NO: 225 | VL-4 | EIVLTQSPATLSLSPGERATLSCSVSSSVSYMHWYQQ |
| | | KPGQAPKLLIYDTSKLASGIPARFSGSGSGTDYTLTIS |
| | | SLEPEDFAVYYCQQWSSNPQYTFGQGTKLEIK |
| SEQ ID NO: 226 | VL-5 | DIVLTQSPLSLPVTPGEPASISCSVSSSVSYMEIWYLQ |
| | | KPGQSPKLLIYDTSKLASGVPDRFSGSGSGTDYTLKI |
| | | SRVEAEDVGVYYCQQWSSNPQYTFGQGTKLEIK |
| SEQ ID NO: 227 | VL-6 | EITLTQSPAFMSATPGDKVNISCSVSSSVSYMHWYQ |
| | | QKPGEAPKFIIYDTSKLASGIPPRFSGSGYGTDYTLTI |
| | | NNIESEDAAYYYCQQWSSNPQYTFGQGTKLEIK |

MH3-2 (murine) also referred to herein as TCRvb5
Binds to human TCRVβ 5-5,5-6

| | | |
|---|---|---|
| SEQ ID NO: 228 | HC CDR1 (Kabat) | SSWMN |
| SEQ ID NO: 229 | HC CDR2 (Kabat) | RIYPGDGDTKYNGKFKG |
| SEQ ID NO: 230 | HC CDR3 (Kabat) | RGTGGWYFDV |
| SEQ ID NO: 231 | HC CDR1 (Chothia) | GYAFSSS |
| SEQ ID NO: 232 | HC CDR2 (Chothia) | YPGDGD |
| SEQ ID NO: 230 | HC CDR3 (Chothia) | RGTGGWYFDV |
| SEQ ID NO: 233 | HC CDR1 (Combined) | GYAFSSSWMN |
| SEQ ID NO: 229 | HC CDR2 (Combined)) | RIYPGDGDTKYNGKFKG |
| SEQ ID NO: 230 | HC CDR3 (Combined) | RGTGGWYFDV |
| SEQ ID NO: 234 | LC CDR1 (Kabat) | RASESVDSSGNSFMH |
| SEQ ID NO: 235 | LC CDR2 (Kabat) | RASNLES |
| SEQ ID NO: 236 | LC CDR3 (Kabat) | QQSFDDPFT |
| SEQ ID NO: 237 | LC CDR1 (Chothia) | SESVDSSGNSF |
| SEQ ID NO: 235 | LC CDR2 (Chothia) | RASNLES |
| SEQ ID NO: 236 | LC CDR3 (Chothia) | QQSFDDPFT |
| SEQ ID NO: 234 | LC CDR1 (Combined) | RASESVDSSGNSFMH |
| SEQ ID NO: 235 | LC CDR2 (Combined) | RASNLES |
| SEQ ID NO: 236 | LC CDR3 (Combined) | QQSFDDPFT |
| SEQ ID NO: 238 | VL | DIVLTQSPASLAVSLGQRATISCRASESVDSSGNSFM |
| | | HWYQQKPGQPPQLLIYRASNLESGIPARFSGSGSRTD |
| | | FTLTINPVEADDVATFYCQQSFDDPFTFGSGTKLEIK |
| SEQ ID NO: 239 | VH | QVQLQQSGPELVKPGASVKISCKASGYAFSSSWMN |
| | | WVKQRPGQGLEWIGRIYPGDGDTKYNGKFKGKATL |
| | | TADKSSSTAYMHLSSLTSVDSAVYFCARRGTGGWY |
| | | FDVWGAGTTVTVSS |

MH3-2 (humanized) VHs
Binds to human TCRVβ 5-5,5-6

| | | |
|---|---|---|
| SEQ ID NO: 240 | VH-1 | QVQLVQSGAEVKKPGASVKVSCKASGYAFSSSWM |
| | | NWVRQAPGQGLEWIGRIYPGDGDTKYNGKFKGRA |
| | | TLTADKSTSTAYMELSSLRSEDTAVYYCARRGTGG |
| | | WYFDVWGQGTTVTVSS |
| SEQ ID NO: 241 | VH-2 | QVQLVQSGAEVKKPGSSVKVSCKASGYAFSSSWMN |
| | | WVRQAPGQGLEWIGRIYPGDGDTKYNGKFKGRATL |
| | | TADKSTSTAYMELSSLRSEDTAVYYCARRGTGGWY |
| | | FDVWGQGTTVTVSS |
| SEQ ID NO: 242 | VH-3 | EVQLVQSGAEVKKPGESLKISCKASGYAFSSSWMN |
| | | WVRQMPGKGLEWIGRIYPGDGDTKYNGKFKGQAT |
| | | LSADKSISTAYLQWSSLKASDTAMYYCARRGTGGW |
| | | YFDVWGQGTTVTVSS |
| SEQ ID NO: 243 | VH-4 | QVQLVQSGSELKKPGASVKVSCKASGYAFSSSWMN |
| | | WVRQAPGQGLEWIGRIYPGDGDTKYNGKFKGRAV |
| | | LSADKSVSMAYLQISSLKAEDTAVYYCARRGTGGW |
| | | YFDVWGQGTTVTVSS |

MH3-2 (humanized) VLs
Binds to human TCRVβ 5-5,5-6

| | | |
|---|---|---|
| SEQ ID NO: 244 | VL-1 | DIVLTQSPDSLAVSLGERATINCRASESVDSSGNSF |
| | | MHWYQQKPGQPPQLLTYRASNLESGVPDRFSGSGS |
| | | RTDFTLTISSLQAEDVAVYYCQQSFDDPFTFGQGTK |
| | | LEIK |
| SEQ ID NO: 245 | VL-2 | EIVLTQSPATLSLSPGERATLSCRASESVDSSGNSFM |
| | | HWYQQKPGQAPQLLIYRASNLESGIPARFSGSGSRT |
| | | DFTLTISSLEPEDFAVYYCQQSFDDPFTFGQGTKLEI |
| | | K |
| SEQ ID NO: 246 | VL-3 | EIVLTQSPATLSLSPGERATLSCRASESVDSSGNSFM |
| | | HWYQQKPGQAPQLLIYRASNLESGIPARFSGSGSRT |
| | | DFTLTISRLEPEDFAVYYCQQSFDDPFTFGQGTKLEI |
| | | K |
| SEQ ID NO: 247 | VL-4 | DIQLTQSPSSLSASVGDRVTITCRASESVDSSGNSFM |
| | | HWYQQKPGKVPQLLIYRASNLESGVPSRFSGSGSRT |
| | | DFTLTISSLQPEDVATYYCQQSFDDPFTFGQGTKLEI |
| | | K |
| SEQ ID NO: 248 | VL-5 | EIVLTQSPDFQSVTPKEKVTITCRASESVDSSGNSFM |
| | | HWYQQKPDQSPQLLIYRASNLESGVPSRFSGSGSRT |
| | | DFTLTINSLEAEDAATYYCQQSFDDPFTFGQGTKLEI |
| | | K |
| SEQ ID NO: 249 | VL-6 | DIVLTQTPLSLPVTPGEPASISCRASESVDSSGNSFMH |
| | | WYLQKPGQSPQLLIYRASNLESGVPDRFSGSGSRTD |
| | | FTLKISRVEAEDVGVYYCQQSFDDPFTFGQGTKLEIK |

ZOE (murine), also referred to as BJ1538
Binds to human TCRVβ 4-1,4-2,4-3

| | | |
|---|---|---|
| SEQ ID NO: 250 | HC CDR1 (Kabat) | DYYMY |
| SEQ ID NO: 251 | HC CDR2 (Kabat) | TISGGGSYTYSPDSVKG |
| SEQ ID NO: 252 | HC CDR3 (Kabat) | ERDIYYGNFNAMVY |
| SEQ ID NO: 253 | HC CDR1 (Chothia) | GFTFSDY |
| SEQ ID NO: 254 | HC CDR2 (Chothia) | SGGGSY |
| SEQ ID NO: 252 | HC CDR3 (Chothia) | ERDIYYGNFNAMVY |
| SEQ ID NO: 255 | HC CDR1 (Combined) | GFTFSDYYMY |
| SEQ ID NO: 251 | HC CDR2 (Combined)) | TISGGGSYTYSPDSVKG |
| SEQ ID NO: 252 | HC CDR3 (Combined) | ERDIYYGNFNAMVY |
| SEQ ID NO: 256 | LC CDR1 (Kabat) | RASKSVSTSGYSYMH |
| SEQ ID NO: 257 | LC CDR2 (Kabat) | LASNLES |
| SEQ ID NO: 258 | LC CDR3 (Kabat) | QHSRDLPWT |
| SEQ ID NO: 259 | LC CDR1 (Chothia) | SKSVSTSGYSY |
| SEQ ID NO: 257 | LC CDR2 (Chothia) | LASNLES |
| SEQ ID NO: 258 | LC CDR3 (Chothia) | QHSRDLPWT |
| SEQ ID NO: 256 | LC CDR1 (Combined) | RASKSVSTSGYSYMH |
| SEQ ID NO: 257 | LC CDR2 (Combined) | LASNLES |
| SEQ ID NO: 258 | LC CDR3 (Combined) | QHSRDLPWT |
| SEQ ID NO: 260 | VL | DIVLTQSPVSLTVSLGQRATISCRASKSVSTSGYSYM |
| | | HWYQQKPGQPPKLLIYLASNLESGVPARFSGSGSGT |
| | | DFTLNIHPVEEEDAATYYCQHSRDLPWTFGGGTKLE |
| | | IK |
| SEQ ID NO: 261 | VH | EVQLVESGGGLVKPGGSLKLSCAASGFTFSDYYMY |
| | | WVRQTPEKRLEWVATISGGGSYTYSPDSVKGRFTIS |
| | | RDNAKNNLYLQMSSLRSEDTAMYFCARERDIYYGN |
| | | FNAMVYWGRGTSVTVSS |

ZOE (humanized) VHs
Binds to human TCRVβ 4-1,4-2,4-3

| | | |
|---|---|---|
| SEQ ID NO: 262 | VH-1 | EVQLLESGGGLVQPGGSLRLSCAASGFTFSDYYMY |
| | | WVRQAPGKGLEWVATISGGGSYTYSPDSVKGRFTIS |
| | | RDNSKNTLYLQMNSLRAEDTAVYYCARERDIYYGN |
| | | FNAMVYWGRGTLVTVSS |
| SEQ ID NO: 263 | VH-2 | EVQLVESGGGLVQPGGSLRLSCAASGFTFSDYYMY |
| | | WVRQAPGKGLEWVATISGGGSYTYSPDSVKGRFTIS |
| | | RDNAKNSLYLQMNSLRAEDTAVYYCARERDIYYGN |
| | | FNAMVYWGRGTLVTVSS |
| SEQ ID NO: 264 | VH-3 | QVQLVESGGGVVQPGRSLRLSCAASGFTFSDYYMY |
| | | WVRQAPGKGLEWVATISGGGSYTYSPDSVKGRFTI |
| | | SRDNSKNTLYLQMNSLRAEDTAVYYCARERDIYY |
| | | GNFNAMVYWGRGTLVTVSS |
| SEQ ID NO: 265 | VH-4 | QVQLVESGGGLVKPGGSLRLSCAASGFTFSDYYMY |
| | | WIRQAPGKGLEWVATISGGGSYTYSPDSVKGRFTIS |
| | | RDNAKNSLYLQMNSLRAEDTAVYYCARERDIYYGN |
| | | FNAMVYWGRGTLVTVSS |

ZOE (humanized) VLs
Binds to human TCRVβ 4-1,4-2,4-3

| | | |
|---|---|---|
| SEQ ID NO: 266 | VL-1 | EIVLTQSPGTLSLSPGERATLSCRASKSVSTSGYSYM |
| | | HWYQQKPGQAPRLLIYLASNLESGIPDRFSGSGSGT |
| | | DFTLTISRLEPEDFAVYYCQHSRDLPWTFGGGTKVEI |
| | | K |
| SEQ ID NO: 267 | VL-2 | EIVLTQSPATLSLSPGERATLSCRASKSVSTSGYSYM |
| | | HWYQQKPGQAPRLLIYLASNLESGIPARFSGSGSGT |
| | | DFTLTISSLEPEDFAVYYCQHSRDLPWTFGGGTKVEI |
| | | K |
| SEQ ID NO: 268 | VL-3 | DIQLTQSPSTLSASVGDRVTITCRASKSVSTSGYSYM |
| | | HWYQQKPGKAPKLLIYLASNLESGVPSRFSGSGSGT |
| | | EFTLTISSLQPDDFATYYCQHSRDLPWTFGGGTKVEI |
| | | K |
| SEQ ID NO: 269 | VL-4 | AIQLTQSPSSLSASVGDRVTITCRASKSVSTSGYSYM |
| | | HWYQQKPGKAPKLLIYLASNLESGVPSRFSGSGSGT |
| | | DFTLTISSLQPEDFATYYCQHSRDLPWTFGGGTKVEI |
| | | K |

Anti-TCRvb19 (murine), also referred to as BJ1465
Binds to human TCRVβ 19

| | | |
|---|---|---|
| SEQ ID NO: 270 | HC CDR1 (Kabat) | GYFWN |
| SEQ ID NO: 271 | HC CDR2 (Kabat) | YISYDGSNNYNPSLKN |
| SEQ ID NO: 272 | HC CDR3 (Kabat) | PSPGTGYAVDY |
| SEQ ID NO: 273 | HC CDR1 (Chothia) | GYSITSGY |
| SEQ ID NO: 274 | HC CDR2 (Chothia) | SYDGSN |
| SEQ ID NO: 272 | HC CDR3 (Chothia) | PSPGTGYAVDY |
| SEQ ID NO: 275 | HC CDR1 (Combined) | GYSITSGYFWN |
| SEQ ID NO: 271 | HC CDR2 (Combined)) | YISYDGSNNYNPSLKN |
| SEQ ID NO: 272 | HC CDR3 (Combined) | PSPGTGYAVDY |
| SEQ ID NO: 276 | LC CDR1 (Kabat) | RSSQSLVHSNGNTYLH |
| SEQ ID NO: 277 | LC CDR2 (Kabat) | KVSNRFS |
| SEQ ID NO: 278 | LC CDR3 (Kabat) | SQSTHVPFT |
| SEQ ID NO: 279 | LC CDR1 (Chothia) | SQSLVHSNGNTY |
| SEQ ID NO: 277 | LC CDR2 (Chothia) | KVSNRFS |
| SEQ ID NO: 278 | LC CDR3 (Chothia) | SQSTHVPFT |
| SEQ ID NO: 276 | LC CDR1 (Combined) | RSSQSLVHSNGNTYLH |
| SEQ ID NO: 277 | LC CDR2 (Combined) | KVSNRFS |
| SEQ ID NO: 278 | LC CDR3 (Combined) | SQSTHVPFT |
| SEQ ID NO: 280 | VL | NVVMTQTPLSLPVSLGDQASISCRSSQSLVHSNGNT |
| | | YLHWYLQKPGQSPKFLIYKVSNRFSGVPDRFSGGGS |
| | | GTEFTLKISRVEAEDLGVYFCSQSTHVPFTFGSGTKL |
| | | EIK |
| SEQ ID NO: 281 | VH | NVQLQESGPGLVKPSQSLSLTCSVAGYSITSGYFWN |
| | | WIRQFPGNKLEWMGYISYDGSNNYNPSLKNRISITR |
| | | DTSKNQFFLKLNSVTTEDTATYYCASPSPGTGYAVD |
| | | YWGQGTSVTVSS |

Anti-TCRvb19 (humanized) VHs
Binds to human TCRVβ 19

| | | |
|---|---|---|
| SEQ ID NO: 282 | VH-1 | QVQLQESGPGLVKPSETLSLTCTVSGYSITSGYFWN |
| | | WIRQPPGKGLEWIGYISYDGSNNYNPSLKNRVTISR |
| | | DTSKNQFSLKLSSVTAADTAVYYCASPSPGTGYAVD |
| | | YWGQGTLVTVSS |
| SEQ ID NO: 283 | VH-2 | QVQLQESGPGLVKPSETLSLTCTVSGYSITSGYFWN |
| | | WIRQPPGKGLEWIGYISYDGSNNYNPSLKNRVTISR |
| | | DTSKNQFSLKLSSVTAADTAVYYCASPSPGTGYAVD |
| | | YWGQGTLVTVSS |
| SEQ ID NO: 284 | VH-3 | QVQLVESGGGLVQPGGSLRLSCSVSGYSITSGYFWN |
| | | WVRQAPGKGLEWVGYISYDGSNNYNPSLKNRFTIS |
| | | RDTSKNTFYLQMNSLRAEDTAVYYCASPSPGTGYA |
| | | VDYWGQGTLVTVSS |

Anti-TCRvb19 (humanized) VLs
Binds to human TCRVβ 19

| | | |
|---|---|---|
| SEQ ID NO: 285 | VL-1 | VVMTQSPGTLSLSPGERATLSCRSSQSLVHSNGNTY |
| | | LHWYQQKPGQAPRFLIYKVSNRFSGIPDRFSGSGSG |
| | | TDFTLTISRLEPEDFAVYFCSQSTHVPFTFGQGTKLEI |
| | | K |
| SEQ ID NO: 286 | VL-2 | EVVMTQSPATLSLSPGERATLSCRSSQSLVHSNGNT |
| | | YLHWYQQKPGQAPRFLIYKVSNRFSGIPARFSGSGS |
| | | GTDFTLTISSLEPEDFAVYFCSQSTHVPFTFGQGTKL |
| | | EIK |
| SEQ ID NO: 287 | VL-3 | EVVMTQSPATLSVSPGERATLSCRSSQSLVHSNGNT |
| | | YLHWYQQKPGQAPRFLIYKVSNRFSGIPARFSGSGS |
| | | GTEFTLTISSLQSEDFAVYFCSQSTHVPFTFGQGTKL |
| | | EIK |
| SEQ ID NO: 288 | VL-4 | DVQMTQSPSSLSASVGDRVTITCRSSQSLVHSNGNT |
| | | YLHWYQQKPGKAPKFLIYKVSNRFSGVPSRFSGSGS |
| | | GTDFTFTISSLQPEDIATYFCSQSTHVPFTFGQGTKLE |
| | | IK |

BL37.2 (murine), also referred to as BJ1539
Binds to human TCRVβ 9

| | | |
|---|---|---|
| SEQ ID NO: 314 | HC CDR1 (Kabat) | DYIVH |
| SEQ ID NO: 315 | HC CDR2 (Kabat) | WINTYTGTPTYADDFEG |
| SEQ ID NO: 316 | HC CDR3 (Kabat) | SWRRGIRGIGFDY |
| SEQ ID NO: 317 | HC CDR1 (Chothia) | GYTFTDY |
| SEQ ID NO: 318 | HC CDR2 (Chothia) | NTYTGT |
| SEQ ID NO: 316 | HC CDR3 (Chothia) | SWRRGIRGIGFDY |
| SEQ ID NO: 319 | HC CDR1 (Combined) | GYTFTDYIVH |
| SEQ ID NO: 315 | HC CDR2 (Combined)) | WINTYTGTPTYADDFEG |
| SEQ ID NO: 316 | HC CDR3 (Combined) | SWRRGIRGIGFDY |
| SEQ ID NO: 320 | LC CDR1 (Kabat) | KASKSINKYLA |
| SEQ ID NO: 321 | LC CDR2 (Kabat) | DGSTLQS |
| SEQ ID NO: 322 | LC CDR3 (Kabat) | QQHNEYPPT |
| SEQ ID NO: 323 | LC CDR1 (Chothia) | SKSINKY |
| SEQ ID NO: 321 | LC CDR2 (Chothia) | DGSTLQS |
| SEQ ID NO: 322 | LC CDR3 (Chothia) | QQHNEYPPT |
| SEQ ID NO: 320 | LC CDR1 (Combined) | KASKSINKYLA |
| SEQ ID NO: 321 | LC CDR2 (Combined) | DGSTLQS |
| SEQ ID NO: 322 | LC CDR3 (Combined) | QQHNEYPPT |
| SEQ ID NO: 324 | VL | DVQMTQSPYNLAASPGESVSINCKASKSINKYLAWY |
| | | QQKPGKPNKLLIYDGSTLQSGIPSRFSGSGSGTDFTL |
| | | TIRGLEPEDFGLYYCQQHNEYPPTFGAGTKLELK |
| SEQ ID NO: 325 | VH | QLQLVQSGPELREPGESVKISCKASGYTFTDYIVHW |
| | | VKQAPGKGLKWMGWINTYTGTPTYADDFEGRFVFS |
| | | LEASASTANLQISNLKNEDTATYFCARSWRRGIRGIG |
| | | FDWGQGVMVTVSS |

BL37.2 (humanized) VH's
Binds to human TCRVβ 9

| | | |
|---|---|---|
| SEQ ID NO: 326 | VH-1 | QLQLVQSGAEVKKPGASVKVSCKASGYTFTDYIVH |
| | | WVRQAPGQGLEWMGWINTYTGTPTYADDFEGWVT |
| | | MTLDASISTAYMELSRLRSDDTAVYYCARSWRRGIR |
| | | GIGFDWGQGTMVTVSS |
| SEQ ID NO: 327 | VH-2 | QLQLVQSGAEVKKPGASVKVSCKASGYTFTDYIVH |
| | | WVRQAPGQGLEWMGWINTYTGTPTYADDFEGRVT |
| | | MTLDASTSTAYMELSSLRSEDTAVYYCARSWRRGI |
| | | RGIGFDWGQGTMVTVSS |
| SEQ ID NO: 328 | VH-3 | QLQLVQSGAEVKKPGASVKVSCKASGYTFTDYIVH |
| | | WVRQAPGQRLEWMGWINTYTGTPTYADDFEGRVTI |
| | | TLDASASTAYMELSSLRSEDMAVYYCARSWRRGIR |
| | | GIGFDWGQGTMVTVSS |
| SEQ ID NO: 329 | VH-4 | QLQLVQSGAEVKKPGASVKVSCKASGYTFTDYIVH |
| | | WVRQATGQGLEWMGWINTYTGTPTYADDFEGRVT |
| | | MTLNASISTAYMELSSLRSEDTAVYYCARSWRRGIR |
| | | GIGFDWGQGTMVTVSS |

BL37.2 (humanized) VL's
Binds to human TCRVβ 9

| | | |
|---|---|---|
| SEQ ID NO: 330 | VL-1 | EVVMTQSPGTLSLSPGERATLSCKASKSINKYLAWY |
| | | QQKPGQAPRLLIYDGSTLQSGIPDRFSGSGSGTDFTL |
| | | TISRLEPEDFAVYYCQQHNEYPPTFGQGTKLEIK |
| SEQ ID NO: 331 | VL-2 | EVVMTQSPATLSLSPGERATLSCKASKSINKYLAWY |
| | | QQKPGQAPRLLIYDGSTLQSGIPARFSGSGSGTDFTL |
| | | TISSLEPEDFAVYYCQQHNEYPPTFGQGTKLEIK |
| SEQ ID NO: 332 | VL-3 | DVQMTQSPSSLSASVGDRVTITCKASKSINKYLAWY |
| | | QQKPGKAPKLLIYDGSTLQSGVPSRFSGSGSGTDFTL |
| | | TISSLQPEDFATYYCQQHNEYPPTFGQGTKLEIK |
| SEQ ID NO: 333 | VL-4 | AVRMTQSPSSFSASTGDRVTITCKASKSINKYLAWY |
| | | QQKPGKAPKLLIYDGSTLQSGVPSRFSGSGSGTDFTL |
| | | TISCLQSEDFATYYCQQHNEYPPTFGQGTKLEIK |

IG125 (murine)
Binds to TRVβ 11-2

| | | |
|---|---|---|
| SEQ ID NO: 334 | HC CDR1 (Kabat) | NYGVH |
| SEQ ID NO: 335 | HC CDR2 (Kabat) | VIWSDGSTDYDTAFIS |
| SEQ ID NO: 336 | HC CDR3 (Kabat) | RAVVADFDY |
| SEQ ID NO: 337 | HC CDR1 (Chothia) | GFSLTN |
| SEQ ID NO: 338 | HC CDR2 (Chothia) | VIWSDGSTD |
| SEQ ID NO: 336 | HC CDR3 (Chothia) | RAVVADFDY |
| SEQ ID NO: 339 | HC CDR1 (Combined) | GFSLTNYGVH |
| SEQ ID NO: 335 | HC CDR2 (Combined) | VIWSDGSTDYDTAFIS |
| SEQ ID NO: 336 | HC CDR3 (Combined) | RAVVADFDY |
| SEQ ID NO: 340 | LC CDR1 (Kabat) | KASKEVTIFGSISALH |
| SEQ ID NO: 341 | LC CDR2 (Kabat) | NGAKLES |
| SEQ ID NO: 342 | LC CDR3 (Kabat) | LQNKEVPFT |
| SEQ ID NO: 340 | LC CDR1 (Chothia) | KASKEVTIFGSISALH |
| SEQ ID NO: 341 | LC CDR2 (Chothia) | NGAKLES |
| SEQ ID NO: 342 | LC CDR3 (Chothia) | LQNKEVPFT |
| SEQ ID NO: 340 | LC CDR1 (Combined) | KASKEVTIFGSISALH |
| SEQ ID NO: 341 | LC CDR2 (Combined) | NGAKLES |
| SEQ ID NO: 342 | LC CDR3 (Combined) | LQNKEVPFT |
| SEQ ID NO: 343 | VH | QVQLKQSGPGLLQPSQSLSITCTVSGFSLTNYGVHW |
| | | VRQSPGKGLEWLGVIWSDGSTDYDTAFISRLSISKD |
| | | NSKSQVFFKLNSLQADDTAIYYCARRAVVADFDYW |
| | | GQGTTLTVSS |
| SEQ ID NO: 344 | VL | DIVLTQSPASLAVSLGQKATISCKASKEVTIFGSISAL |
| | | HWYQQKPGQPPKLIYNGAKLESGVSARFSDSGSQN |
| | | RSPFGNQLSFTLTIAPVEADDAATYYCLQNKEVPFT |
| | | FGSGTKLEIK |

IG125 (humanized) VHs
Binds to TRVβ 11-2

| | | |
|---|---|---|
| SEQ ID NO: 345 | VH-1 | QVTLKESGPVLVKPTETLTLTCTVSGFSLTNYGVH |
| | | WVRQPPGKALEWLGVIWSDGSTDYDTAFISRLTISK |
| | | DNSKSQVVLTMTNMDPVDTATYYCARRAVVADF |
| | | DYWGQGTTVTVSS |
| SEQ ID NO: 346 | VH-2 | QVQLQESGPGLVKPSGTLSLTCAVSGFSLTNYGVH |
| | | WVRQPPGKGLEWLGVIWSDGSTDYDTAFISRLTISK |
| | | DNSKSQVSLKLSSVTAADTAVYYCARRAVVADFD |
| | | YWGQGTTVTVSS |
| SEQ ID NO: 347 | VH-3 | QVQLQQSGPGLVKPSQTLSLTCAVSGFSLTNYGVH |
| | | WVRQSPSRGLEWLGVIWSDGSTDYDTAFISRLTINK |
| | | DNSKSQVSLQLNSVTPEDTAVYYCARRAVVADFD |
| | | YWGQGTTVTVSS |
| SEQ ID NO: 348 | VH-4 | EVQLVESGGGLVQPGPSLRLSCTVSGFSLTNYGVH |
| | | WVRQAPGKGLEWLGVIWSDGSTDYDTAFISRLTIS |
| | | KDNSKSIVYLQMNSLKTEDTAVYYCARRAVVADF |
| | | DYWGQGTTVTVSS |
| SEQ ID NO: 349 | VH-5 | EVQLVQSGAEVKKPGESLRISCKVSGFSLTNYGVH |
| | | WVRQMPGKGLEWLGVIWSDGSTDYDTAFISQLTIS |
| | | KDNSISTVYLQWSSLKASDTAMYYCARRAVVADF |
| | | DYWGQGTTVTVSS |

IG125 (humanized) VLs
Binds to TRVβ 11-2

| | | |
|---|---|---|
| SEQ ID NO: 350 | VL-1 | DIVLTQSPDSLAVSLGERATINCKASKEVTIFGSISAL |
| | | HWYQQKPGQPPKLLYNGAKLESGVSARFGVPDRFS |
| | | RSGSGLDFTLTISSLQAEDVAVYYCLQNKEVPFTFG |
| | | QGTKLEIK |
| SEQ ID NO: 351 | VL-2 | EIVLTQSPDFQSVTPKEKVTITCKASKEVTIFGSISAL |
| | | HWYQQKPDQSPKLLYNGAKLESGVSARFGVPSRFS |
| | | RSGSGLDFTLTINSLEAEDAATYYCLQNKEVPFTFG |
| | | QGTKLEIK |
| SEQ ID NO: 352 | VL-3 | AIQLTQSPSSLSASVGDRVTITCKASKEVTIFGSISAL |
| | | HWYQQKPGKAPKLLYNGAKLESGVSARFGVPSRFS |
| | | RSGSGLDFTLTISSLQPEDFATYYCLQNKEVPFTFGQ |
| | | GTKLEIK |
| SEQ ID NO: 353 | VL-4 | DIVLTQTPLSLSVTPGQPASISCKASKEVTIFGSISAL |
| | | HWYLQKPGQPPKLLYNGAKLESGVSARFGVPDRFS |
| | | RSGSGLDFTLKISRVEAEDVGVYYCLQNKEVPFTFG |
| | | QGTKLEIK |

Medi-1

| | | |
|---|---|---|
| SEQ ID NO: 354 | HC CDR1 | TFTEN |
| SEQ ID NO: 355 | HC CDR2 | IDPEDGTTDYVEKFKN |
| SEQ ID NO: 356 | HC CDR3 | GVGSGDYVMDA |
| SEQ ID NO: 357 | LC CDR1 | RASQSVSISRHNLIH |
| SEQ ID NO: 358 | LC CDR2 | RASNLAS |
| SEQ ID NO: 359 | LC CDR3 | QQSGESPRT |

Medi-2

| | | |
|---|---|---|
| SEQ ID NO: 360 | VL | LSLGQRATISCRASQSVSISRHNLIHWYQQKPGQQP |
| | | KLLIYRASNLASGIPARFSGSGSGTDFTLTINPVQAD |
| | | DVATYYCQQSGESPRTFGGGTKLELK |
| SEQ ID NO: 361 | VH | GRGRAQSVVVQASGYTFTENYIYWVKQRPKQGLE |
| | | LIGRIDPEDGTTDYVEKFKNKATLTVDTSSKTAYM |
| | | QLSSLTSEDTASYFCARGVGSGDYVMDAWGQGAS |
| | | VTVSS |

Medi-3

| | | |
|---|---|---|
| SEQ ID NO: 362 | HC CDR1 | GYTFTAYYIS |
| SEQ ID NO: 363 | HC CDR2 | RIDPEDGSTDYVEKFK |
| SEQ ID NO: 364 | HC CDR3 | GNSDYVMDA |
| SEQ ID NO: 365 | LC CDR1 | RASQSVSISGINLMH |
| SEQ ID NO: 366 | LC CDR2 | RASSLAS |
| SEQ ID NO: 367 | LC CDR3 | QQSWESPRT |

Medi-4

| | | |
|---|---|---|
| SEQ ID NO: 368 | HC CDR1 | GYTFTAYY |
| SEQ ID NO: 369 | HC CDR2 | IDPEDGST |
| SEQ ID NO: 370 | HC CDR3 | CARGNSD |
| SEQ ID NO: 371 | LC CDR1 | QSVSISGINL |
| SEQ ID NO: 366 | LC CDR2 | RASSLAS |
| SEQ ID NO: 372 | LC CDR3 | CQQSWESPRT |

Medi-5

| | | |
|---|---|---|
| SEQ ID NO: 373 | VL | LCLRQRATISCRASQSVSISGINLMHWYQQRPGQQP |
| | | KLLIYRASSLASGIPARFSGRGSGTDFTLTIDPVQAD |
| | | DIAAYFCQQSWESPRTF GGGTQLELKR |
| SEQ ID NO: 374 | VH | DWNSRTGLSQVSCKASGYTFTAYYISWVKQRPKQ |
| | | GLELIG RIDPEDGSTD |
| | | YVEKFKIKATLTADTSSNTAYMQFSSLTFEDTATYF |
| | | CARGNSDYVMDAWGQGASVTVSS |

TABLE 6
Constant region amino acid sequences of human IgG heavy chains and
human kappa light chain.

| | | |
|---|---|---|
| Human kappa | LC | RTVAAPSVFIFPPSDEQLKSGTASVVCLLNNFYPREAKVQWKVDNAL |
| constant region | | QSGNSQESVTEQDSKDSTYSLSSTLTLSKADYEKHKVYACEVTHQGL |
| SEQ ID NO: 289 | | SSPVTKSFNRGEC |
| IgG4 (S228P) | HC | ASTKGPSVFPLAPCSRSTSESTAALGCLVKDYFPEPVTVSWNSGALTS |
| mutant constant | | GVHTFPAVLQSSGLYSLSSVVTVPSSSLGTKTYTCNVDHKPSNTKVD |
| region (EU | | KRVESKYGPPCPPCPAPEFLGGPSVFLFPPKPKDTLMISRTPEVTCVVV |
| Numbering) | | DVSQEDPEVQFNWYVDGVEVHNAKTKPREEQFNSTYRVVSVLTVLH |
| SEQ ID NO: 290 | | QDWLNGKEYKCKVSNKGLPSSIEKTISKAKGQPREPQVYTLPPSQEE |
| | | MTKNQVSLTCLVKGFYPSDIAVEWESNGQPENNYKTTPPVLDSDGSF |
| | | FLYSRLTVDKSRWQEGNVFSCSVMHEALHNHYTQKSLSLSLG |
| IgG1 wild type | HC | ASTKGPSVFPLAPSSKSTSGGTAALGCLVKDYFPEPVTVSWNSGALTS |
| SEQ ID NO: 291 | | GVHTFPAVLQSSGLYSLSSVVTVPSSSLGTQTYICNVNHKPSNTKVDK |
| | | RVEPKSCDKTHTCPPCPAPELLGGPSVFLFPPKPKDTLMISRTPEVTCV |
| | | VVDVSHEDPEVKFNWYVDGVEVHNAKTKPREEQYNSTYRVVSVLT |
| | | VLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |
| IgG1 (N297A) | HC | ASTKGPSVFPLAPSSKSTSGGTAALGCLVKDYFPEPVTVSWNSGALTS |
| mutant constant | | GVHTFPAVLQSSGLYSLSSVVTVPSSSLGTQTYICNVNHKPSNTKVDK |
| region (EU | | RVEPKSCDKTHTCPPCPAPELLGGPSVFLFPPKPKDTLMISRTPEVTCV |
| Numbering) | | VVDVSHEDPEVKFNWYVDGVEVHNAKTKPREEQYASTYRVVSVLT |
| SEQ ID NO: 292 | | VLHQDWLNGKEYKCKVSNKALPAPIEKTISKAKGQPREPQVYTLPPS |
| | | REEMTKNQVSLTCLVKGFYPSDIAVEWESNGQPENNYKTTPPVLDSD |
| | | GSFFLYSKLTVDKSRWQQGNVFSCSVMHEALHNHYTQKSLSLSPGK |

### Chimeric Antigen Receptors (CARs)

In some embodiments, the T cells described herein express a chimeric antigen receptor (CAR) and are referred to herein as CAR T cells. In some embodiments, the methods described herein comprise introducing one or more exogenous nucleic acid molecules encoding a chimeric antigen receptor (CAR) into a population of T cells. In some embodiments, the one or more exogenous nucleic acid molecules encoding a chimeric antigen receptor (CAR) are introduced into a population of T cells post expansion by a method described herein. In some embodiments, the one or more exogenous nucleic acid molecules encoding a chimeric antigen receptor (CAR) are introduced into a population of T cells prior to expansion by a method described herein.

In some embodiments, a CAR polypeptide comprises an extracellular region (ectodomain) that comprises an antigen binding region, a transmembrane region and, optionally an intracellular (endodomain) region. In some embodiments, the intracellular region further comprises one or more intracellular signaling regions. In some embodiments, a CAR described herein comprises an antigen binding region, a transmembrane region, one or more costimulatory regions or domains, and a signaling region for T-cell activation.

In some embodiments, an antigen binding region comprises complementary determining regions of a monoclonal antibody (e.g., three heavy chain CDRs and three light chain CDRs), variable regions of a monoclonal antibody, and/or antigen binding fragments thereof. In some instances, an antigen binding region comprises F(ab′)₂, Fab′, Fab, Fv, or scFv. In some embodiments, an antigen binding region is a scFv. In some embodiments, an antigen binding region is a Fab. In some embodiments, an antigen binding region is a Fab′. In some embodiments, an antigen binding region is F(ab′)₂. In some embodiments, an antigen binding region is an Fv.

In some embodiments, a CAR comprises an antigen binding region that binds to an epitope of CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WTI), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1).

In some embodiments, a polypeptide comprises a transmembrane region or transmembrane domain derived from either a natural or a synthetic source. Where the source is natural, the region can be derived from any membrane-bound or transmembrane protein. Suitable transmembrane regions can include, but not limited to, the transmembrane region(s) of alpha, beta or zeta chain of the T-cell receptor; or a transmembrane region from CD28, CD3 epsilon, CD3ζ, CD45, CD4, CD5, CD8alpha, CD9, CD16, CD22, CD33, CD37, CD64, CD80, CD86, CD134, CD137, CD152 (CTLA-4) or CD154. Alternatively, the transmembrane region or domain can be synthetic, and can comprise hydrophobic residues such as leucine and valine. In some embodiments, a triplet of phenylalanine, tryptophan and valine is found at one or both termini of a synthetic transmembrane domain. Optionally, a short oligonucleotide or polypeptide linker, in some embodiments, between 2 and 10 amino acids in length may form the linkage between the transmembrane domain and the cytoplasmic signaling domain of a CAR. In some embodiments, the linker is a glycine-serine linker. In some embodiments, the transmembrane region comprises a CD8a transmembrane domain, a CD152 (CTLA-4), a TCRα, TCRβ, a TCRγ1, a TCRδ or a CD3ζ transmembrane domain.

In some embodiments, the CAR comprises an intracellular region. In some embodiments, said intracellular region comprises a primary signaling domain. Exemplary primary signaling domains include, but are not limited to, intracellular domain of CD3ζ, FcR gamma, FcR beta, CD3 gamma, CD3 delta, CD3 epsilon, CD5, CD22, CD79a, CD79b or CD66d. In some embodiments, the primary signaling domain comprises intracellular domain of CD3ζ. In some embodiments, said intracellular region comprises a primary signaling domain and one or more costimulatory domains. Exemplary costimulatory domains include, but are not limited to, CD3ζ, CD8, CD27, CD28, 4-1BB (CD137), ICOS, DAP10, DAP12, OX40 (CD134) or functional fragments or variants thereof, or any combination thereof. In some instances, a CAR described herein comprises two, three, four, or five costimulatory domains.

In some embodiments, provided herein are chimeric antigen receptors that do not contain a CD3ζ signaling domain. In some embodiments, (a) an antigen binding domain, wherein the antigen binding domain does not contain a T cell receptor α (TCRα) variable region, a T cell receptor β (TCRβ) variable region, or both (b) a transmembrane domain; and (c) an intracellular signaling domain comprising a TCRβ constant region intracellular signaling domain, wherein the CAR does not contain a CD3t intracellular signaling domain. In some embodiments, (a) an antigen binding domain, wherein the antigen binding domain is a single chain variable fragment (scFv) or a single domain antibody; (b) a transmembrane domain; and (c) an intracellular signaling domain comprising a TCRβ intracellular signaling domain, and wherein the CAR does not contain a CD3ζ intracellular signaling domain.

In some embodiments, absence of a CD3ζ signaling domain in a CAR prevents cytokine release syndrome induced by infusion of a population of immune effector cells (e.g., T cells and NK cells). In some embodiments, absence of a CD3ζ signaling domain in a CAR prevents cytokine release syndrome induced by infusion of a population of immune effector cells (e.g., T cells and NK cells), wherein antigen presenting cells release a lower level of one or more proinflammatory cytokine (e.g., IL-6, IFNγ, TNFα, IL-6, IL-1β, IL-8, IL-10, sIL2Rα, sgp130, sIL6R, MCP1, MIP1α, MIP1β, and GM-CSF). In some embodiments, absence of a CD3ζ signaling domain in a CAR prevents cytokine release syndrome induced by infusion of a population of immune effector cells (e.g., T cells and NK cells), wherein the immune effector cells expressing the CAR release a lower level of one or more proinflammatory cytokine (e.g., IFNγ, TNFα, IL-6, IL-1β, IL-8, IL-10, sIL2Rα, sgp130, sIL6R, MCP1, MIP1α, MIP1β, and GM-CSF). In some embodiments, the CAR comprises a TCRβ intracellular domain.

In some embodiments, a nucleic acid molecule encoding an CAR described herein is introduced into a T cell using a vector. In some embodiments, the vector is a plasmid, viral vector, or non-viral vector. In some embodiments, the viral vector is a lentivirus vector, adenovirus vector, adeno-associated virus vector, or a retrovirus vector. In some embodiments, the nucleic acid molecule encoding the CAR is introduced into the cell population by transfection or transduction. In some embodiments, the nucleic acid molecule is integrated into the host genome. In some embodiments, the nucleic acid molecule is integrated into the host genome by transposon/transposase system; CRISPR system, a zinc finger nuclease system, or Talen system. In some embodiments, the CRISPR system comprises at least one gRNA and an endonuclease (e.g., Cas9). In some embodiments, the nucleic acid molecule is integrated into the host genome through a viral vector (e.g., a lentivirus vector, adenovirus vector, adeno-associated virus vector, or a retrovirus vector).

In some embodiments, a nucleic acid encoding said CAR is integrated into the host genome. In some embodiments, the nucleic acid is targeted for integration at a specific genomic locus. In some embodiments, the nucleic acid is targeted for integration in a TRAC or TCRB gene sequence. In some embodiments, the nucleic acid is targeted for integration within an immune checkpoint gene sequence (e.g., an immune checkpoint gene described herein). In some embodiments, the nucleic acid not targeted for integration at a specific genomic locus.

### Exogenous T Cell Receptors (TCRs)

In some embodiments, the T cells described herein express and exogenous T cells receptor. In some embodiments, the methods described herein comprise introducing one or more nucleic acid molecules encoding an exogenous T cell receptor (TCR). In some embodiments, the one or more nucleic acid molecules encoding the exogenous T cell receptor are introduced into a population of T cells after the T cells have been expanded by a method described herein. In some embodiments, the one or more nucleic acid molecules encoding the exogenous T cell receptor are introduced into a population of T cells prior the T cells have been expanded by a method described herein.

T cell receptors are composed of two chains (αβ or γδ) that pair on the surface of the T cell to form a heterodimeric receptor (αβ pair or γδ pair). Each chain (α, β, γ, and δ) are composed of two domains: a constant domain (C) which anchors the protein to the cell membrane and is associated with invariant subunits of the CD3 signaling apparatus; and a variable domain (V) that confers antigen recognition through six loops, referred to as complementarity determining regions (CDRs). In some instances, each of the V domains comprises three CDRs; e.g., CDR1, CDR2 and CDR3 with CDR3 as the hypervariable region. These CDRs interact with a complex formed between an antigenic peptide bound to a protein encoded by the major histocompatibility complex (pepMHC) (e.g., HLA-A, HLA-B, HLA-C, HLA-DPA1, HLA-DPB1, HLA-DQA1, HLA-DQB1, HLA-DRA, or HLA-DRB1 complex). In some instances, the constant domain further comprises a joining region that connects the constant domain to the variable domain. In some cases, the beta chain further comprises a short diversity region which makes up part of the joining region.

In some embodiments, said exogenous T cell receptors bind to a peptide/MHC complex, wherein said peptide is derived from CD19, CD123, CD22, CD30, CD171, CS-1, C-type lectin-like molecule-1, CD33, epidermal growth factor receptor variant III (EGFRvIII), ganglioside G2 (GD2), ganglioside GD3, TNF receptor family member B cell maturation (BCMA), Tn antigen ((Tn Ag) or (GalNAcα-Ser/Thr)), prostate-specific membrane antigen (PSMA), Receptor tyrosine kinase-like orphan receptor 1 (ROR1), Fms-Like Tyrosine Kinase 3 (FLT3), Tumor-associated glycoprotein 72 (TAG72), CD38, CD44v6, Carcinoembryonic antigen (CEA), Epithelial cell adhesion molecule (EPCAM), B7H3 (CD276), KIT (CD117), Interleukin-13 receptor subunit alpha-2, mesothelin, Interleukin 11 receptor alpha (IL-11Ra), prostate stem cell antigen (PSCA), Protease Serine 21, vascular endothelial growth factor receptor 2 (VEGFR2), Lewis(Y) antigen, CD24, Platelet-derived growth factor receptor beta (PDGFR-beta), Stage-specific embryonic antigen-4 (SSEA-4), CD20, Folate receptor alpha, Receptor tyrosine-protein kinase ERBB2 (Her2/neu), Mucin 1, cell surface associated (MUC1), epidermal growth factor receptor (EGFR), neural cell adhesion molecule (NCAM), Prostase, prostatic acid phosphatase (PAP), elongation factor 2 mutated (ELF2M), Ephrin B2, fibroblast activation protein alpha (FAP), insulin-like growth factor 1 receptor (IGF-I receptor), carbonic anhydrase IX (CAIX), Proteasome (Prosome, Macropain) Subunit, Beta Type, 9 (LMP2), glycoprotein 100 (gp100), oncogene fusion protein consisting of breakpoint cluster region (BCR) and Abelson murine leukemia viral oncogene homolog 1 (Abl) (bcr-abl), tyrosinase, ephrin type-A receptor 2 (EphA2), Fucosyl GM1, sialyl Lewis adhesion molecule (sLe), ganglioside GM3, transglutaminase 5 (TGS5), high molecular weight-melanoma-associated antigen (HMWMAA), o-acetyl-GD2 ganglioside (OAcGD2), Folate receptor beta, tumor endothelial marker 1 (TEM1/CD248), tumor endothelial marker 7-related (TEM7R), claudin 6 (CLDN6), thyroid stimulating hormone receptor (TSHR), G protein-coupled receptor class C group 5, member D (GPRC5D), chromosome X open reading frame 61 (CXORF61), CD97, CD179a, anaplastic lymphoma kinase (ALK), Polysialic acid, placenta-specific 1 (PLAC1), hexasaccharide portion of globoH glycoceramide (GloboH), mammary gland differentiation antigen (NY-BR-1), uroplakin 2 (UPK2), Hepatitis A virus cellular receptor 1 (HAVCR1), adrenoceptor beta 3 (ADRB3), pannexin 3 (PANX3), G protein-coupled receptor 20 (GPR20), lymphocyte antigen 6 complex, locus K 9 (LY6K), Olfactory receptor 51E2 (OR51E2), TCR Gamma Alternate Reading Frame Protein (TARP), Wilms tumor protein (WTI), Cancer/testis antigen 1 (NY-ESO-1), Cancer/testis antigen 2 (LAGE-1a), Melanoma-associated antigen 1 (MAGE-A1), ETS translocation-variant gene 6, located on chromosome 12p (ETV6-AML), sperm protein 17 (SPA17), X Antigen Family, Member 1A (XAGE1), angiopoietin-binding cell surface receptor 2 (Tie 2), melanoma cancer testis antigen-1 (MAD-CT-1), melanoma cancer testis antigen-2 (MAD-CT-2), Fos-related antigen 1, tumor protein p53 (p53), p53 mutant, prostein, surviving, telomerase, prostate carcinoma tumor antigen-1, melanoma antigen recognized by T cells 1, Rat sarcoma (Ras) mutant, human Telomerase reverse transcriptase (hTERT), sarcoma translocation breakpoints, melanoma inhibitor of apoptosis (ML-IAP), ERG (transmembrane protease, serine 2 (TMPRSS2) ETS fusion gene), N-Acetyl glucosaminyl-transferase V (NA17), paired box protein Pax-3 (PAX3), Androgen receptor, Cyclin B1, v-myc avian myelocytomatosis viral oncogene neuroblastoma derived homolog (MYCN), Ras Homolog Family Member C (RhoC), Tyrosinase-related protein 2 (TRP-2), Cytochrome P450 1B1 (CYP1B1), CCCTC-Binding Factor (Zinc Finger Protein)-Like, Squamous Cell Carcinoma Antigen Recognized By T Cells 3 (SART3), Paired box protein Pax-5 (PAX5), proacrosin binding protein sp32 (OY-TES1), lymphocyte-specific protein tyrosine kinase (LCK), A kinase anchor protein 4 (AKAP-4), synovial sarcoma, X breakpoint 2 (SSX2), Receptor for Advanced Glycation Endproducts (RAGE-1), renal ubiquitous 1 (RU1), renal ubiquitous 2 (RU2), legumain, human papilloma virus E6 (HPV E6), human papilloma virus E7 (HPV E7), intestinal carboxyl esterase, heat shock protein 70-2 mutated (mut hsp70-2), CD79a, CD79b, CD72, Leukocyte-associated immunoglobulin-like receptor 1 (LAIR1), Fc fragment of IgA receptor (FCAR or CD89), Leukocyte immunoglobulin-like receptor subfamily A member 2 (LILRA2), CD300 molecule-like family member f (CD300LF), C-type lectin domain family 12 member A (CLEC12A), bone marrow stromal cell antigen 2 (BST2), EGF-like module-containing mucin-like hormone receptor-like 2 (EMR2), lymphocyte antigen 75 (LY75), Glypican-3 (GPC3), Fc receptor-like 5 (FCRL5), or immunoglobulin lambda-like polypeptide 1 (IGLL1). In some embodiments, the exogenous TCR bind to a cancer antigen expressed within a patient's tumor (i.e. patient-specific, somatic, non-synonymous mutations expressed by tumors) in the context of MHC. In some embodiments, the exogenous TCR bind to a cancer neoantigens expressed within a patient's tumor (i.e. patient-specific, somatic, non-synonymous mutations expressed by tumors) in the context of MHC. In some embodiments, engineered TCRs are affinity-enhanced.

In some embodiments, a TCR is described using the International Immunogenetics (IMGT) TCR nomenclature, and links to the IMGT public database of TCR sequences. For example, there can be several types of alpha chain variable (Vα) regions and several types of beta chain variable (Vβ) regions distinguished by their framework, CDR1, CDR2, and CDR3 sequences. As such, a Vα type can be referred to in IMGT nomenclature by a unique TRAV number. For example, “TRAV21” defines a TCR Vα region having unique framework and CDR1 and CDR2 sequences, and a CDR3 sequence which is partly defined by an amino acid sequence which is preserved from TCR to TCR but which also includes an amino acid sequence which varies from TCR to TCR. Similarly, “TRBV5-1” defines a TCR Vβ region having unique framework and CDR1 and CDR2 sequences, but with only a partly defined CDR3 sequence. In some cases, the beta chain diversity region is referred to in IMGT nomenclature by the abbreviation TRBD. In some instances, the unique sequences defined by the IMGT nomenclature are widely known and accessible to those working in the TCR field. For example, they can be found in the IMGT public database and in “T cell Receptor Factsbook,” (2001) LeFranc and LeFranc, Academic Press, ISBN 0-12-441352-8.

In some embodiments, an αβ heterodimeric TCR is transfected as full length chains having both cytoplasmic and transmembrane domains. In some embodiments, the TCRs contain an introduced disulfide bond between residues of the respective constant domains, as described, for example, in WO 2006/000830.

In some embodiments, TCRs described herein are in single chain format, for example see WO2004/033685. Single chain formats include αβ TCR polypeptides of the Vα-L-Vβ, Vβ-L-Vα, Vα-Cα-L-Vβ, Vα-L-Vβ-Cβ, Vα-Cα-L-Vβ-Cβ types, wherein Vα and Vβ are TCR α and β variable regions respectively, Cα and Cβ are TCR α and β constant regions respectively, and L is a linker sequence. In certain embodiments single chain TCRs of the invention may have an introduced disulfide bond between residues of the respective constant domains, as described in WO2004/033685, incorporated by reference herein.

In some embodiments, a nucleic acid molecule encoding an exogenous TCR described herein is introduced into a T cell using a vector. In some embodiments, the vector is a plasmid, viral vector, or non-viral vector. In some embodiments, the viral vector is a lentivirus vector, adenovirus vector, adeno-associated virus vector, or a retrovirus vector. In some embodiments, the nucleic acid molecule encoding the exogenous TCR is introduced into the cell population by transfection or transduction. In some embodiments, the nucleic acid molecule is integrated into the host genome. In some embodiments, the nucleic acid molecule is integrated into the host genome by transposon/transposase system; CRISPR system, a zinc finger nuclease system, or Talen system. In some embodiments, the CRISPR system comprises at least one gRNA and an endonuclease (e.g., Cas9). In some embodiments, the nucleic acid molecule is integrated into the host genome through a viral vector (e.g., a lentivirus vector, adenovirus vector, adeno-associated virus vector, or a retrovirus vector).

In some embodiments, a nucleic acid encoding said exogenous T cell receptor is integrated into the host genome. In some embodiments, the nucleic acid is targeted for integration at a specific genomic locus. In some embodiments, the nucleic acid is targeted for integration in a TRAC or TCRB gene sequence. In some embodiments, the nucleic acid is targeted for integration within an immune checkpoint gene sequence (e.g., an immune checkpoint gene described herein). In some embodiments, the nucleic acid not targeted for integration at a specific genomic locus.

Exemplary TCR sequences are disclosed in Table 7.

TABLE 7
Exemplary TCR sequences

| TCR Domain | Amino Acid Sequence | SEQ ID NO: |
|---|---|---|
| TCRα Constant | IQNPDPAVYQLRDSKSSDKSVCLFTDFDSQTNVSQSKDS | 293 |
| domain | DVYITDKTVLDMRSMDFKSNSAVAWSNKSDFACANAF | |
| | NNSIIPEDTFFPSPESSCDVKLVEKSFETDTNLNFQNLS | |
| TCRα | VIGFRILLLKVAGFNLLMTLRLW | 294 |
| Transmembrane | | |
| domain | | |
| TCRα Intracellulasr | SS | 295 |
| domain | | |
| TCRβ Constant 1 | PEVAVFEPSEAEISHTQKATLVCLATGFFPDHVELSWWV | 296 |
| domain (no signal | NGKEVHSGVSTDPQPLKEQPALNDSRYCLSSRLRVSATF | |
| sequence) | WQNPRNHFRCQVQFYGLSENDEWTQDRAKPVT | |
| TCRβ Constant 1 | DLNKVFPPEVAVFEPSEAEISHTQKATLVCLATGFFPDHV | 297 |
| domain (with signal | ELSWWVNGKEVHSGVSTDPQPLKEQPALNDSRYCLSSR | |
| sequence) | LRVSATFWQNPRNHFRCQVQFYGLSENDEWTQDRAKP | |
| | VTQIVSAEAWGRADCGFTSVSYQQGVLSATILYE | |
| TCRβ Constant 1 | ILLGKATLYAVLVSALVLMAM | 298 |
| Transmembrane | | |
| domain | | |
| TCRβ Constant 1 | VKRKDF | 299 |
| Intracellular | | |
| domain | | |
| TCRβ Constant 2 | PKVAVFEPSEAEISHTQKATLVCLATGFYPDHVELSWWV | 300 |
| domain (no signal | NGKEVHSGVSTDPQPLKEQPALNDSRYCLSSRLRVSATF | |
| sequence) | WQNPRNHFRCQVQFYGLSENDEWTQDRAKPVTQIVSAE | |
| | AWGRADCGFTSESYQQGVLSA | |
| TCRβ Constant 2 | DLKNVFPPKVAVFEPSEAEISHTQKATLVCLATGFYPDH | 301 |
| domain (with signal | VELSWWVNGKEVHSGVSTDPQPLKEQPALNDSRYCLSS | |
| sequence) | RLRVSATFWQNPRNHFRCQVQFYGLSENDEWTQDRAKP | |
| | VTQIVSAEAWGRADCGFTSESYQQGVLSA | |
| TCRβ Constant 2 | TILYEILLGKATLYAVLVSALVL | 302 |
| Transmembrane | | |
| domain | | |
| TCRβ Constant 2 | MAMVKRKDSRG | 303 |
| Intracellular | | |
| domain | | |

### Heterologous Targeting Constructs

In some embodiments, the T cells described herein express a heterologous targeting construct that comprises an extracellular antigen-binding domain and a transmembrane domain operatively linked to the antigen binding domain, wherein the heterologous targeting construct lacks an intracellular domain capable of activating the cell. In some embodiments, the construct further comprises a talk domain operatively linking the antigen-binding domain to the transmembrane domain. In some embodiments, the antigen-binding domain comprises a single chain variable fragment (scFv), a monoclonal antibody, a Fab fragment, a B cell receptor, a T cell receptor, an antibody scaffold, a receptor-specific ligand, or a ligand-specific receptor. In some embodiments, clustering of the heterologous targeting construct upon binding of the antigen-binding domain to a target antigen does not substantially activate the TCR pathway in the engineered. In some embodiments, the antigen-binding domain binds a tumor-associated antigen (e.g., described herein). In some embodiments, binding of the antigen-binding domain to a target antigen expressed on a healthy cell triggers substantially less cytolysis by the engineered T cell relative to a reference cell having a functional intracellular domain. In some embodiments, binding of the antigen-binding domain to the target antigen expressed on a healthy cell does not substantially trigger cytolysis by the engineered T cell. In some embodiments, binding of the antigen-binding domain to a target antigen expressed on a tumor cell or an infected cell substantially triggers cytolysis by the engineered T cell.

### Immune Checkpoint Proteins

In some embodiments, the T cells described herein comprise a genomic alteration that results in decreased or completely inhibited expression of an immune check point protein. In some embodiments, said immune checkpoint protein is normally expressed on the surface of the cell. In some embodiments, said immune checkpoint protein is normally expressed intracellularly. In some embodiments, said immune checkpoint protein is selected from the group consisting adenosine A2a receptor (ADORA), Cytokine-inducible SH2-containing protein (CISH), CD276, V-set domain containing T cell activation inhibitor 1 (VTCN1), B and T lymphocyte associated (BTLA), indoleamine 2,3-dioxygenase 1 (IDO1), killer cell immunoglobulin-like receptor, three domains, long cytoplasmic tail, 1 (KIR3DL1), lymphocyte-activation gene 3 (LAG3), hepatitis A virus cellular receptor 2 (HAVCR2), V-domain immunoglobulin suppressor of T-cell activation (VISTA), natural killer cell receptor 2B4 (CD244), hypoxanthine phosphoribosyltransferase 1 (HPRT), adeno-associated virus integration site 1(AAVS1), or chemokine (C-C motif) receptor 5 (gene/pseudogene) (CCR5), CD160 molecule (CD160), T-cell immunoreceptor with Ig and ITIM domains (TIGIT), CD96 molecule (CD96), cytotoxic and regulatory T-cell molecule (CRTAM), leukocyte associated immunoglobulin like receptor 1(LAIR1), sialic acid binding Ig like lectin 7 (SIGLEC7), sialic acid binding Ig like lectin 9 (SIGLEC9), tumor necrosis factor receptor superfamily member 10b (TNFRSF10B), tumor necrosis factor receptor superfamily member 10a (TNFRSF10A), caspase 8 (CASP8), caspase 10 (CASP10), caspase 3 (CASP3), caspase 6 (CASP6), caspase 7 (CASP7), Fas associated via death domain (FADD), Fas cell surface death receptor (FAS), transforming growth factor beta receptor II (TGFBRII), transforming growth factor beta receptor I (TGFBR1), SMAD family member 2 (SMAD2), SMAD family member 3 (SMAD3), SMAD family member 4 (SMAD4), SKI proto-oncogene (SKI), SKI-like proto-oncogene (SKIL), TGFB induced factor homeobox 1(TGIF1), programmed cell death 1 (PD-1), cytotoxic T-lymphocyte-associated protein 4 (CTLA4), interleukin 10 receptor subunit alpha (IL10RA), interleukin 10 receptor subunit beta (IL10RB), heme oxygenase 2 (HMOX2), interleukin 6 receptor (IL6R), interleukin 6 signal transducer (IL6ST), c-src tyrosine kinase (CSK), phosphoprotein membrane anchor with glycosphingolipid microdomains 1(PAG1), signaling threshold regulating transmembrane adaptor 1(SIT1), forkhead box P3 (FOXP3), PR domain 1(PRDM1), basic leucine zipper transcription factor, ATF-like (BATF), guanylate cyclase 1, soluble, alpha 2 (GUCY1A2), guanylate cyclase 1, soluble, alpha 3 (GUCY1A3), guanylate cyclase 1, soluble, beta 2(GUCY1B2), prolyl hydroxylase domain (PHD1, PHD2, PHD3) family of proteins, or guanylate cyclase 1, soluble, beta 3 (GUCY1B3), egl-9 family hypoxia-inducible factor 1 (EGLN1), egl-9 family hypoxia-inducible factor 2 (EGLN2), egl-9 family hypoxia-inducible factor 3 (EGLN3), and protein phosphatase 1 regulatory subunit 12C (PPP1R12C).

### Linkers

The term “linker” as used in the context of polypeptides refers to a peptide linker that consists of amino acids that link two regions of a polypeptide together. In some embodiments, the linker comprises or consists of glycine residues, serine residues, or glycine and serine residues. In some embodiments, the linker is a Gly/Ser linker and comprises the amino acid sequence (Gly-Gly-Gly-Ser)n (SEQ ID NO: 375), where n is a positive integer equal to or greater than 1. For example, n=1, n=2, n=3, n=4, n=5 and n=6, n=7, n=8, n=9 and n=10 In some embodiments, the linker comprises (Gly₄Ser)₄ (SEQ ID NO: 376) or (Gly₄Ser)₃ (SEQ ID NO: 377). In some embodiments, the linker comprises multiple repeats of (Gly₂Ser), (GlySer) or (Gly₃Ser) (SEQ ID NO: 378). Also included within the scope are linkers described in WO2012/138475, incorporated herein by reference).

Other exemplary linkers include, but are not limited to the following amino acid sequences:

(SEQ ID NO: 304)
GGG; DGGGS;
(SEQ ID NO: 305)
TGEKP;
(SEQ ID NO: 306)
GGRR;
(SEQ ID NO: 307)
(GGGGS)ₙ;
wherein = 1, 2, 3, 4 or 5
(SEQ ID NO: 308)
EGKSSGSGSESKVD;
(SEQ ID NO: 309)
KESGSVSSEQLAQFRSLD;
(SEQ ID NO: 310)
GGRRGGGS;
(SEQ ID NO: 311)
LRQRDGERP;
(SEQ ID NO: 312)
LRQKDGGGSERP;
(SEQ ID NO: 313)
LRQKD(GGGS)₂ ERP.

Alternatively, flexible linkers can be rationally designed using a computer program capable of modeling both DNA-binding sites and the peptides themselves or by phage display methods.

### Methods of Use and Pharmaceutical Compositions

Generally, T cells activated and expanded as described herein may be utilized in the treatment of various diseases. Pharmaceutical compositions may be administered in a manner appropriate to the disease to be treated. The quantity and frequency of administration will be determined by such factors as the condition of the patient, and the type and severity of the patient's disease, although appropriate dosages may be determined by clinical trials.

In some embodiments, methods described herein can be used to manufacture cells expressing polynucleotides and/or polypeptides for the treatment of a hyperproliferative disease, such as a cancer, an autoimmune disease or for the treatment of an infection, such as a viral, bacterial or parasitic infection. In some embodiments, the antigen is an antigen that is elevated in cancer cells, in autoimmune cells or in cells that are infected by a virus, bacteria or parasite. Pathogens that may be targeted include, without limitation, Plasmodium, trypanosome, Aspergillus, Candida, Hepatitis A, Hepatitis B, Hepatitis C, HSV, HPV, RSV, EBV, CMV, JC virus, BK virus, or Ebola pathogens. Autoimmune diseases can include graft-versus-host disease, rheumatoid arthritis, lupus, celiac disease, Crohn's disease, Sjogren Syndrome, polymyalgia rheumatic, multiple sclerosis, neuromyelitis optica, ankylosing spondylitis, Type 1 diabetes, alopecia areata, vasculitis, temporal arteritis, bullous pemphigoid, psoriasis, pemphigus vulgaris or autoimmune uveitis.

In some embodiments, the disease is a cancer or infection. In some embodiments, the cancer is acute lymphoblastic leukemia, acute lymphocytic leukemia, acute myelogenous leukemia, aplastic anemia, chronic myelogenous leukemia, desmoplastic small round cell tumor, Ewing's sarcoma, Hodgkin's disease, multiple myeloma, myelodysplasia, Non-Hodgkin's lymphoma, paroxysmal nocturnal hemoglobinuria, radiation poisoning, chronic lymphocytic leukemia, AL amyloidosis, essential thrombocytosis, polycythemia vera, severe aplastic anemia, neuroblastoma, breast tumors, ovarian tumors, renal cell carcinoma, autoimmune disorders, such as systemic sclerosis, osteopetrosis, inherited metabolic disorders, juvenile chronic arthritis, adrenoleukodystrophy, amegakaryocytic thrombocytopenia, sickle cell disease, severe congenital immunodeficiency, Griscelli syndrome type II, Hurler syndrome, Kostmann syndrome, Krabbe disease, metachromatic leukodystrophy, thalassemia, hemophagocytic lymphohistiocytosis, and Wiskott-Aldrich syndrome, leukemias, lymphomas, melanomas, neuroendocrine tumors, carcinomas and sarcomas. Exemplary cancers that may be treated with a compound, pharmaceutical composition, or method provided herein include lymphoma, sarcoma, bladder cancer, bone cancer, brain tumor, cervical cancer, colon cancer, esophageal cancer, gastric cancer, head and neck cancer, kidney cancer, myeloma, thyroid cancer, leukemia, prostate cancer, breast cancer (e.g. triple negative, ER positive, ER negative, chemotherapy resistant, herceptin resistant, HER2 positive, doxorubicin resistant, tamoxifen resistant, ductal carcinoma, lobular carcinoma, primary, metastatic), ovarian cancer, pancreatic cancer, liver cancer (e.g., hepatocellular carcinoma), lung cancer (e.g. non-small cell lung carcinoma, squamous cell lung carcinoma, adenocarcinoma, large cell lung carcinoma, small cell lung carcinoma, carcinoid, sarcoma), glioblastoma multiforme, glioma, melanoma, prostate cancer, castration-resistant prostate cancer, breast cancer, triple negative breast cancer, glioblastoma, ovarian cancer, lung cancer, squamous cell carcinoma (e.g., head, neck, or esophagus), colorectal cancer, leukemia, acute myeloid leukemia, lymphoma, B cell lymphoma, or multiple myeloma. Additional examples include, cancer of the thyroid, endocrine system, brain, breast, cervix, colon, head & neck, esophagus, liver, kidney, lung, non-small cell lung, melanoma, mesothelioma, ovary, sarcoma, stomach, uterus or Medulloblastoma, Hodgkin's Disease, Non-Hodgkin's Lymphoma, multiple myeloma, neuroblastoma, glioma, glioblastoma multiforme, ovarian cancer, rhabdomyosarcoma, primary thrombocytosis, primary macroglobulinemia, primary brain tumors, cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, Paget's Disease of the Nipple, Phyllodes Tumors, Lobular Carcinoma, Ductal Carcinoma, cancer of the pancreatic stellate cells, cancer of the hepatic stellate cells, or prostate cancer. In some embodiments, the cancer is a solid tumor. In some embodiments, the cancer is hematological.

In some embodiments, the infection is a fungal, bacterial, or viral infection. Exemplary pathogens include those of the families of Adenoviridae, Epstein-Barr virus (EBV), Cytomegalovirus (CMV), Respiratory Syncytial Virus (RSV), JC virus, BK virus, HSV, HHV family of viruses, Picornaviridae, Herpesviridae, Hepadnaviridae, Flaviviridae, Retroviridae, Orthomyxoviridae, Paramyxoviridae, Papovaviridae, Polyomavirus, Rhabdoviridae, and Togaviridae. Exemplary pathogenic viruses cause smallpox, influenza, mumps, measles, chicken pox, ebola, and rubella. Exemplary pathogenic fungi include Candida, Aspergillus, Cryptococcus, Histoplasma, Pneumocystis, and Stachybotrys. Exemplary pathogenic bacteria include Streptococcus, Pseudomonas, Shigella, Campylobacter, Staphylococcus, Helicobacter, E. coli, Rickettsia, Bacillus, Bordetella, Chlamydia, Spirochetes, and Salmonella. In some embodiments the pathogen receptor Dectin-1 may be used to generate a CAR that recognizes the carbohydrate structure on the cell wall of fungi such as Aspergillus.

The immune response induced in a subject by administering T cells activated and expanded wherein T cells are stimulated and expanded to therapeutic levels, may include cellular immune responses mediated by cytotoxic T cells, capable of killing tumor and infected cells, regulatory T cells, and helper T cell responses. Humoral immune responses, mediated primarily by helper T cells capable of activating B cells thus leading to antibody production, may also be induced. A variety of techniques may be used for analyzing the type of immune responses induced by the compositions, which are well described in the art; e.g., Coligan et al. Current Protocols in Immunology, John Wiley & Sons Inc. (1994).

It can generally be stated that a pharmaceutical composition comprising the T cells described herein may be administered at a dosage of 10⁴ to 10⁹cells/kg body weight, preferably 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988). The optimal dosage and treatment regime for a particular patient can readily be determined by one skilled in the art of medicine by monitoring the patient for signs of disease and adjusting the treatment accordingly.

Typically, in adoptive immunotherapy studies, antigen-specific T cells are administered approximately at 2×10⁹ to 2×10¹¹ cells to the patient. (See, e.g., U.S. Pat. No. 5,057,423). In some aspects, particularly in the use of allogeneic or xenogeneic cells, lower numbers of cells, in the range of 10⁶/kilogram (10⁶-10¹¹ per patient) may be administered. In certain embodiments, T cells are administered at 1×10⁵, 1×10⁶, 1×10⁷, 1×10⁸, 2×10⁸, 2×10⁹, 1×10¹⁰, 2×10¹⁰, 1×10¹¹, 5×10¹¹, or 1×10¹² cells to the subject. T cell compositions may be administered multiple times at dosages within these ranges. The cells may be autologous or heterologous to the patient undergoing therapy. If desired, the treatment may also include administration of mitogens (e.g., PHA) or lymphokines, cytokines, and/or chemokines (e.g., GM-CSF, IL-4, IL-7, IL-13, FIt3-L, RANTES, MIP1α, etc.) as described herein to enhance induction of the immune response.

In certain embodiments, it may be desired to administer activated T cells to a subject and then subsequently redraw blood (or have a leukapheresis performed), activate T cells therefrom, and reinfuse the patient with these activated and expanded T cells. This process can be carried out multiple times every few weeks. In certain embodiments, T cells can be activated from blood draws of from 10 cc to 400 cc. In certain embodiments, T cells are activated from blood draws of 20 cc, 30 cc, 40 cc, 50 cc, 60 cc, 70 cc, 80 cc, 90 cc, or 100 cc. Not to be bound by theory, using this multiple blood draw/multiple reinfusion protocol, may select out certain populations of T cells.

The administration of the subject compositions may be carried out in any convenient manner, including by aerosol inhalation, injection, ingestion, transfusion, implantation or transplantation. The compositions described herein may be administered to a patient subcutaneously, intradermally, intratumorally, intranodally, intramedullary, intramuscularly, by intravenous (i. v.) injection, or intraperitoneally. In one embodiment, the T cell compositions are administered to a patient by intradermal or subcutaneous injection. In another embodiment, the T cell compositions are preferably administered by i.v. injection. The compositions of T cells may be injected directly into a tumor, lymph node, or site of infection.

In yet another embodiment, the pharmaceutical composition can be delivered in a controlled release system. In one embodiment, a pump may be used (see Langer, 1990, Science 249:1527-1533; Sefton 1987, CRC Crit. Ref. Biomed. Eng. 14:201; Buchwald et al., 1980; Surgery 88:507; Saudek et al., 1989, N. Engl. J. Med. 321:574). In another embodiment, polymeric materials can be used (see Medical Applications of Controlled Release, 1974, Langer and Wise (eds.), CRC Pres., Boca Raton, Fla.; Controlled Drug Bioavailability, Drug Product Design and Performance, 1984, Smolen and Ball (eds.), Wiley, New York; Ranger and Peppas, 1983; J. Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105). In yet another embodiment, a controlled release system can be placed in proximity of the therapeutic target, thus requiring only a fraction of the systemic dose (see, e.g., Medical Applications of Controlled Release, 1984, Langer and Wise (eds.), CRC Pres., Boca Raton, Fla., vol. 2, pp. 115-138).

The T cell compositions may also be administered using any number of matrices. Matrices have been utilized for a number of years within the context of tissue engineering (see, e.g., Principles of Tissue Engineering (Lanza, Langer, and Chick (eds.)), 1997. The type of matrix that may be used in the compositions, devices and methods is virtually limitless and may include both biological and synthetic matrices. In one particular example, the compositions and devices set forth by U.S. Pat. Nos. 5,980,889; 5,913,998; 5,902,745; 5,843,069; 5,787,900; or 5,626,561 are utilized. Matrices comprise features commonly associated with being biocompatible when administered to a mammalian host. Matrices may be formed from both natural or synthetic materials. The matrices may be non-biodegradable in instances where it is desirable to leave permanent structures or removable structures in the body of an animal, such as an implant; or biodegradable. The matrices may take the form of sponges, implants, tubes, telfa pads, fibers, hollow fibers, lyophilized components, gels, powders, porous compositions, or nanoparticles. In addition, matrices can be designed to allow for sustained release seeded cells or produced cytokine or other active agent. In certain embodiments, the matrix is flexible and elastic, and may be described as a semisolid scaffold that is permeable to substances such as inorganic salts, aqueous fluids and dissolved gaseous agents including oxygen.

In certain embodiments, cells activated and expanded using the methods described herein, or other methods known in the art where T cells are expanded to therapeutic levels, are administered to a patient in conjunction with (e.g., before, simultaneously or following) any number of relevant treatment modalities, including but not limited to treatment with agents such as antiviral therapy, cidofovir and interleukin-2, Cytarabine (also known as ARA-C) or natalizumab treatment for MS patients or efalizumab treatment for psoriasis patients or other treatments for PML patients. In further embodiments, the T cells may be used in combination with chemotherapy, radiation, immunosuppressive agents, such as cyclosporin, azathioprine, methotrexate, mycophenolate, and FK506, antibodies, or other immunoablative agents such as CAM PATH, anti-CD3 antibodies or other antibody therapies, cytoxin, fludaribine, cyclosporin, FK506, rapamycin, mycophenolic acid, steroids, FR901228, cytokines, and irradiation. These drugs inhibit either the calcium dependent phosphatase calcineurin (cyclosporine and FK506) or inhibit the p70S6 kinase that is important for growth factor induced signaling (rapamycin). (Liu et al., Cell 66:807-815, 1991; Henderson et al., Immun. 73:316-321, 1991; Bierer et al., Curr. Opin. Immun. 5:763-773, 1993; Isoniemi (supra)). In a further embodiment, the cell compositions are administered to a patient in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH. In another embodiment, the cell compositions are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells. In an additional embodiment, expanded cells are administered before or following surgery.

The dosage of the above treatments to be administered to a patient will vary with the precise nature of the condition being treated and the recipient of the treatment. The scaling of dosages for human administration can be performed according to art-accepted practices. The dose for CAMPATH, for example, will generally be in the range 1 to about 100 mg for an adult patient, usually administered daily for a period between 1 and 30 days. The preferred daily dose is 1 to 10 mg per day although in some instances larger doses of up to 40 mg per day may be used (described in U.S. Pat. No. 6,120,766).

In one embodiment the expanded antigen-specific T cell population obtained is biased to producing a CD8+ cell population. In one embodiment the expanded antigen-specific T cell population obtained is biased to producing a CD4+ cell population. In one embodiment the process of the present disclosure is employed to provide a cell population comprising a CD4+ T cell population, for example a Th1 population. A Th1 population as employed herein is intended to refer to a CD4+ population wherein 5% of the cells or more, such as 10, 20, 30, 40, 50, 60, 70, 80, 90% or more are classified as Th1. Memory T cells are a component of Th1 cells. In one embodiment the process of the present disclosure is employed to provide a cell population comprising a CD8+ T cell population

In one embodiment the population of cells obtained from the process comprises a sub-population of memory T cells, for example the memory T cells represent 10, 20, 30, 40, 50 or 60% of the expanded cells and will generally express effector memory markers such as CD27, CD28, CD62L and CD45RO. This will be significantly higher than the population of memory cells prior to expansion.

In some embodiments, residual CD3−, CD56+, and NK cells in the final cell population are acceptable since these are potentially beneficial.

In some embodiments, he cell populations expanded using a process of the present disclosure comprise a desired T cell population and a minimal percentage of non-desired cell population. In some embodiments, the final product administered to the patient comprises a minimal percentage of other cells that the process did not target the expansion of. In some embodiments the final product comprises at least 90%, 95%, 98%, 99%, or 100% of the desired CD4+ and/or CD8+ T cell population. Frequency of the cell populations may be measured, for example, by employing a cytokine assay (e.g., IFNγ ELISPOT assay) or by measuring expression of cell surface proteins, which is known to persons skilled in the art.

In some embodiments a T cells population obtained from a process described herein is diverse when analyzed by serotyping, and without the emergence of dominant clone. In some embodiments, the T cell diversity in the starting sample is substantially represented in the expanded T cells, i.e. the expansion is not generally the expansion of a single clone. In some embodiments a T cells population obtained from a process described herein is not diverse when analyzed by serotyping, characterized by the emergence of a dominant clone.

In some embodiments the T cell population produced by a method described herein comprises a plurality of T cells that express a T cell receptor on the surface. In some embodiments, a T cell population made by a method described herein have one or more advantageous properties in comparison to cells prepared using activation/expansion with an anti-CD3ε antibody. In some embodiments, the one or more advantageous properties comprise less or no production of cytokines associated with cytokine release syndrome (CRS), e.g., IL-6, IL-1beta and TNF alpha; and enhanced and/or delayed production of IL-2 and IFNγ, compared to a method of preparing cells using activation/expansion with an anti-CD3ε antibody. For example, in some embodiments, IL-6 production can be at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 26-fold, 27-fold, 28-fold, 29-fold, 30-fold, 31-fold, 32-fold, 33-fold, 34-fold, 35-fold, 36-fold, 37-fold, 38-fold, 39-fold, 39-fold, 40-fold, 41-fold, 42-fold, 43-fold, 44-fold, 45-fold, 46-fold, 47-fold, 48-fold, 49-fold or at least 50-fold less than T cells prepared using activation/expansion with an anti-CD3ε antibody. For example, in some embodiments, IL-1beta production can be at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 26-fold, 27-fold, 28-fold, 29-fold, 30-fold, 31-fold, 32-fold, 33-fold, 34-fold, 35-fold, 36-fold, 37-fold, 38-fold, 39-fold, 39-fold, 40-fold, 41-fold, 42-fold, 43-fold, 44-fold, 45-fold, 46-fold, 47-fold, 48-fold, 49-fold or at least 50-fold less than T cells prepared using activation/expansion with an anti-CD3ε antibody. For example, in some embodiments, TNF alpha production can be at least 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 26-fold, 27-fold, 28-fold, 29-fold, 30-fold, 31-fold, 32-fold, 33-fold, 34-fold, 35-fold, 36-fold, 37-fold, 38-fold, 39-fold, 39-fold, 40-fold, 41-fold, 42-fold, 43-fold, 44-fold, 45-fold, 46-fold, 47-fold, 48-fold, 49-fold or at least 50-fold less than T cells prepared using activation/expansion with an anti-CD3ε antibody. In some embodiments, an enhancement of IL-2 of at least 1.1-fold, 2-fold, 5-fold, 10-fold or about 20-fold or about 50-fold may be observed in T cells prepared by a method described herein, over T cells prepared using activation/expansion with an anti-CD3ε antibody. In some embodiments, a delay, e.g., at least 1, 2, 3, 4, 5, 6, 7, 8, 9, 10 or more hours delay, in increased level, e.g., expression level, and/or activity of IL-2 may be observed. In some embodiments, a delay, e.g., at least 1, 2, 3, 4, 5, 6, 7, 8, 9, 10 hours delay, in increased level, e.g., expression level, and/or activity of IFNγ may be observed. In some embodiments, the one or more advantageous properties include limiting the unwanted side-effects of CRS, e.g., CRS associated with anti-CD3ε targeting.

Advantageous properties include, but are not limited to, lower levels of IFNγ secretion, in vivo proliferation, up-regulation of a T cell activation marker (for example T cell receptors) may be high relative to the total number of antigen specific T cells in the population.

In some embodiments, T cells prepared by methods described herein show enhanced antigen specificity in comparison to cells prepared using activation/expansion with an anti-CD3ε antibody.

In some embodiments, T cells prepared by methods described herein show comparable avidity (not significantly different) to cell populations prepared using activation/expansion with an anti-CD3ε antibody.

In some embodiments, the therapeutic T cell population administered to a subject and made by a method disclosed herein may technically be a sub-therapeutic dose in the composition. However, after infusion into the subject the cells expand further analysis of whether the T cells are suitable for expanding in vivo may be tested employing an in vitro test, for example using a cell proliferation assay, for example the CF SE assay. Cell proliferation may be assayed by labelling cells with fluorescent compound CF SE to monitor division to a given stimulus. In short cells are labelled with CFSE and antigen is added which stimulates some cells to divide. These cells can be monitored as when they divide the amount of dye in each daughter cell is halved thus halving the brightness of the cell as detected by flow cytometry. Therefore, the number of divisions the cell population has undergone can be determined. In some embodiments, the expanded T cells are capable of further expansion in vitro and in vivo, significant levels expansion for example include 2, 3, 4, 5 fold expansion or more. In some embodiments, at least 70% of the relevant cells are viable as measured by dye exclusion or flow cytometry, for example at least 75%, 80%, 85%, 90%, 95%, 96%, 97%, 98%, or 99% of the cells are viable.

In some embodiments, once the final formulation has been prepared it will be filled into a suitable container, for example an infusion bag or cryovial. In some embodiments, the process according to the present disclosure comprises the further step of filling the T cell population or pharmaceutical formulation thereof into a suitable container, such as an infusion bag and sealing the same. In some embodiments, the container filled with the T cell population of the present disclosure or a pharmaceutical composition comprising the same is frozen for storage and transport, for example is store at about −135° C. In some embodiments, the process of the present disclosure comprises the further step of freezing the T cell population of the present disclosure or a pharmaceutical composition comprising the same. In some embodiments, the “product” is frozen by reducing the temperature by 1° C. per minute to ensure the crystals formed do not disrupt the cell structure. This process may be continued until the sample has reached about −100° C. A product according to the present disclosure is intended to refer to a cultured cell population of the present disclosure or a pharmaceutical composition comprising the same. In some embodiments the product is transferred, shipped, transported in a frozen form to the patient's location. In some embodiments the product according to the present disclosure is provided in a form suitable for parenteral administration, for example, infusion, slow injection or bolus injection. In one embodiment the formulation is provided in a form suitable for intravenous infusion. In some embodiments, the present disclosure provides a method of transport a product according to the present disclosure, from the place of manufacture, or a convenient collection point to the vicinity of the intended patient, for example where the T cell product is stored at or below 0° C. during transit, such as below −100° C. In some embodiments, a protein stabilizing agent is added to the cell culture after manufacturing, for example albumin, in particular human serum album, which may act as a stabilizing agent. The amounts albumin employed in the formulation may be 1 to 50% w/w, for example 10 to 50% w/w, such as about 2.25, 4.5 or 12.5% w/w. In some embodiments the formulation also contains a cryopreservative, for example glycerol or DMSO. The quantity of DMSO is generally 12% or less such as about 10% w/w. In some embodiments the process comprises the further step of preparing a pharmaceutical formulation by adding a pharmaceutically acceptable excipient, in particular an excipient as described herein, for example diluent, stabilizer and/or preservative. Excipient as employed herein is a generic term to cover all ingredients added to the T cell population that do not have a biological or physiological function.

In some embodiments, T cells produced by a method described herein have an average cell diameter which is 95% or less, for example 90% or less, such as 85% or less, more specifically 80% or less of the maximum cell diameter. In some embodiments, the average cell diameter of cells in the relevant T cell population is in the range 10 to 14 microns and the average cell diameter is about 10, 11, 12, 13 or 14 microns.

### CRS Grading

Methods described herein include, methods of preventing or lessening the severity of cytokine release syndrome (CRS) in a human subject. In some embodiments, the method comprises: removing a plurality of T cells from a human subject, expanding the plurality of T cells from the human subject comprising contacting the plurality of T cells to a first agent, wherein the first agent comprises a domain that specifically binds to a T cell receptor beta variable chain (TCRβV) region, thereby generating a first population of T cells, infusing at least a portion of the first population of T cells into the human subject, wherein the subject shows no symptoms of CRS or less severe symptoms (e.g., one or more symptom described herein) of CRS relative to a human subject infused with at least a first population of T cells generated by removing a plurality of T cells the subject and expanding the plurality of T cells by contacting the plurality of T cells with an agent that binds CD3 (e.g., CD3e).

In some embodiments, methods described herein include administering cells made by the methods described herein to a subject. In some embodiments, CRS is prevented. In some embodiments, the subject has no or less severe CRS. In some embodiments, the subject as no or less severe CRS after 1, 2, 3, 4, 5, 6, 7, 8, 9, 10, 14, 21, 30 or more days post administration of the cells. CRS can be graded in severity from 1-5 as follows. Grades 1-3 are less than severe CRS. Grades 4-5 are severe CRS. For Grade 1 CRS, only symptomatic treatment is needed (e.g., nausea, fever, fatigue, myalgias, malaise, headache) and symptoms are not life threatening. For Grade 2 CRS, the symptoms require moderate intervention and generally respond to moderate intervention. Subjects having Grade 2 CRS develop hypotension that is responsive to either fluids or one low-dose vasopressor; or they develop grade 2 organ toxicity or mild respiratory symptoms that are responsive to low flow oxygen (<40% oxygen). In Grade 3 CRS subjects, hypotension generally cannot be reversed by fluid therapy or one low-dose vasopressor. These subjects generally require more than low flow oxygen and have grade 3 organ toxicity (e.g., renal or cardiac dysfunction or coagulopathy) and/or grade 4 transaminitis. Grade 3 CRS subjects require more aggressive intervention, e.g., oxygen of 40% or higher, high dose vasopressor(s), and/or multiple vasopressors. Grade 4 CRS subjects suffer from immediately life-threatening symptoms, including grade 4 organ toxicity or a need for mechanical ventilation. Grade 4 CRS subjects generally do not have transaminitis. In Grade 5 CRS subjects, the toxicity causes death. Sets of criteria for grading CRS are provided herein as Table 8, Table 9, and Table 10. Unless otherwise specified, CRS as used herein refers to CRS according to the criteria of Table 9. In embodiments, CRS is graded according to Table 8. In embodiments, CRS is graded according to Table 9. In embodiments, CRS is graded according to Table 10.

TABLE 8
CRS grading

| | |
|---|---|
| Gr1 | Supportive care only |
| Gr2 | IV therapies +/− hospitalization. |
| Gr3 | Hypotension requiring IV fluids or |
| | low-dose vasoactives or hypoxemia |
| | requiring oxygen, CPAP, or BIPAP. |
| Gr4 | Hypotension requiring high-dose |
| | vasoactives or hypoxemia requiring |
| | mechanical ventilation. |
| Gr5 | Death |

TABLE 9
CTCAE v 4.0 CRS grading scale

| CRS grade | Characteristics |
|---|---|
| Grade 1 | Mild; No infusion interruption; No intervention |
| Grade 2 | Infusion interruption indicated but responds |
| | promptly to symptomatic treatment (e.g., |
| | antihistamines, NSAIDS, narcotics, IV fluids); |
| | prophylactic medications indicated for <= |
| | 24 hrs |
| Grade 3 | Prolonged (e.g., not rapidly responsive to |
| | symptomatic medications and/or brief |
| | interruption of infusion); recurrence of symptoms |
| | following initial improvement; hospitalization |
| | indicated for clinical sequelae (e.g., renal |
| | impairment, pulmonary infiltrates) |
| Grade 4 | Life threatening consequences; pressor or ventilator |
| | support |

TABLE 10
NCI CRS grading scale

| CRS grade | Characteristics |
|---|---|
| Grade 1 | Symptoms are not life threatening and require |
| | symptomatic treatment only; e.g., fever, nausea, |
| | fatigue, headache, myalgias, malaise |
| Grade 2 | Symptoms require and respond to moderate intervention; |
| | Oxygen requirement < 40% or hypotension responsive to |
| | fluids or low dose pressors or Grade 2 organ toxicity |
| Grade 3 | Symptoms require and respond to aggressive intervention; |
| | Oxygen requirement >= 40% or Hypotension requiring |
| | high dose or multiple pressors or grade 3 organ toxicity |
| | or grade 4 transaminitis |
| Grade 4 | Life threatening symptoms Requirement for ventilator |
| | support or Grade 4; organ toxicity (excluding transaminitis) |

### Macrophage Activation Syndrome, a Neurological Toxicity, and Tumor Lysis Syndrome

Methods described herein include, methods of preventing or lessening the severity of macrophage activation syndrome, a neurological toxicity, or tumor lysis syndrome in a human subject.

In some embodiments, the method comprises: removing a plurality of T cells from a human subject, expanding the plurality of T cells from the human subject comprising contacting the plurality of T cells to a first agent, wherein the first agent comprises a domain that specifically binds to a T cell receptor beta variable chain (TCRβV) region, thereby generating a first population of T cells, infusing at least a portion of the first population of T cells into the human subject, wherein the subject shows no symptoms of macrophage activation syndrome or less severe symptoms (e.g., one or more symptom described herein) of macrophage activation syndrome relative to a human subject infused with at least a first population of T cells generated by removing a plurality of T cells the subject and expanding the plurality of T cells by contacting the plurality of T cells with an agent that binds CD3 (e.g., CD3e). Symptoms of macrophage activation syndrome include, but are not limited to, fever, headache, lymphadenopathy, hepatosplenomegaly, coagulopathy, rash, tachycardia, arrhythmia, cardiomyopathy, lethargy, pancytopenia, liver dysfunction, disseminated intravascular coagulation, hypofibrinogenemia, hyperferritinemia, or hypertriglyceridemia. In some embodiments, the at least one symptom is fever, headache, lymphadenopathy, hepatosplenomegaly, coagulopathy, rash, tachycardia, arrhythmia, cardiomyopathy, lethargy, pancytopenia, liver dysfunction, disseminated intravascular coagulation, hypofibrinogenemia, hyperferritinemia, or hypertriglyceridemia.

In some embodiments, the method comprises: removing a plurality of T cells from a human subject, expanding the plurality of T cells from the human subject comprising contacting the plurality of T cells to a first agent, wherein the first agent comprises a domain that specifically binds to a T cell receptor beta variable chain (TCRβV) region, thereby generating a first population of T cells, infusing at least a portion of the first population of T cells into the human subject, wherein the subject shows no symptoms of a neurological toxicity or less severe symptoms (e.g., one or more symptom described herein) of a neurological toxicity relative to a human subject infused with at least a first population of T cells generated by removing a plurality of T cells the subject and expanding the plurality of T cells by contacting the plurality of T cells with an agent that binds CD3 (e.g., CD3e). Symptoms of a neurological toxicity include, but are not limited to, encephalopathy, aphasia, tremor, ataxia, hemiparesis, palsy, dysmetria, seizure, motor weakness, loss of consciousness, or cerebral edema. In some embodiments, the at least one symptom is encephalopathy, aphasia, tremor, ataxia, hemiparesis, palsy, dysmetria, seizure, motor weakness, loss of consciousness, or cerebral edema.

In some embodiments, the method comprises: removing a plurality of T cells from a human subject, expanding the plurality of T cells from the human subject comprising contacting the plurality of T cells to a first agent, wherein the first agent comprises a domain that specifically binds to a T cell receptor beta variable chain (TCRβV) region, thereby generating a first population of T cells, infusing at least a portion of the first population of T cells into the human subject, wherein the subject shows no symptoms of tumor lysis syndrome or less severe symptoms (e.g., one or more symptom described herein) of tumor lysis syndrome relative to a human subject infused with at least a first population of T cells generated by removing a plurality of T cells the subject and expanding the plurality of T cells by contacting the plurality of T cells with an agent that binds CD3 (e.g., CD3e). Symptoms of a neurological toxicity include, but are not limited to, nausea, vomiting, diarrhea, muscle cramps, muscle twitches, weakness, numbness, tingling, fatigue, lethargy, decreased urination, encephalopathy, aphasia, tremor, ataxia, hemiparesis, palsy, dysmetria, seizure, motor weakness, loss of consciousness, cerebral edema, or hallucinations. In some embodiments, the at least one symptom is nausea, vomiting, diarrhea, muscle cramps, muscle twitches, weakness, numbness, tingling, fatigue, lethargy, decreased urination, encephalopathy, aphasia, tremor, ataxia, hemiparesis, palsy, dysmetria, seizure, motor weakness, loss of consciousness, cerebral edema, or hallucinations.

### EXAMPLES

### Example 1: Characteristics of Anti-TCRβV Antibodies

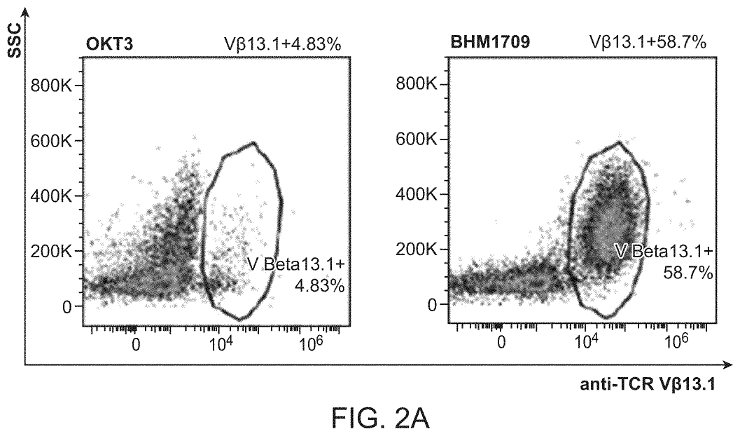

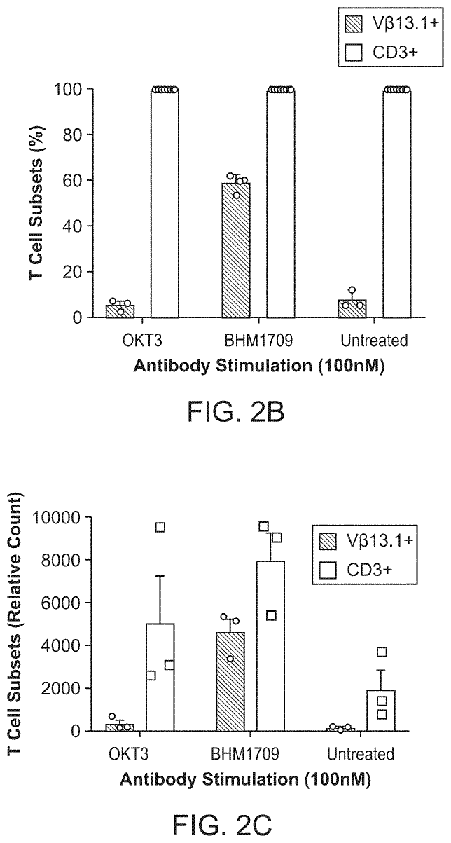

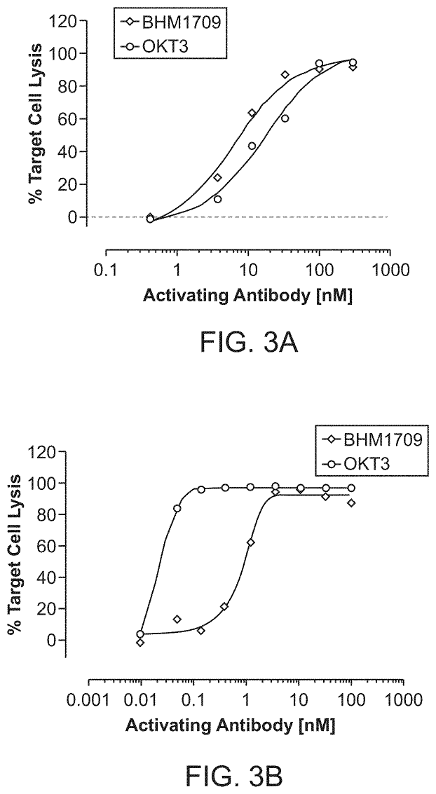

Human CD3+ T cells were isolated using magnetic-bead separation (negative selection) and activated with immobilized (plate-coated) BHM1709 or OKT3 (anti-CD3ε) antibodies at 100 nM for 6 days. T cells, defined by positive staining with BHM1709, were expanded (from ˜5% of T cells on day 0 to almost 60% of total T cells on day 6 of cell culture) (FIGS. 2A-2C). The expanded Vb13.1+ T cells display cytolytic activity against transformed cell line RPMI-8226 when co-cultured with purified CD3+ T cells (FIGS. 3A-3B).

The cytokine production of PBMCs activated with anti-TCRβV antibodies was compared to the cytokine production of PBMCs activated with: (i) anti-CD3ε antibodies (OKT3 or SP34-2); (ii) anti-TCRAV antibodies (anti-TCRAV 12.1 antibody 6D6.6, anti-TCRAV 24JA18 antibody 6B11); (iii) anti-TCR αβ antibody (T10B9); or (iv) isotype control (BGM0109). The anti-TCRβV antibodies tested included: humanized anti-TCRβV 13.1 antibodies BHM1709 and BHM 1710, murine anti-TCRβV 5 antibody MH3-2, murine anti-TCRβV 8.1 antibody 16G8, and murine anti-TCRβV 12 antibody S511. Supernatant samples were taken at days 1, 2, 3, 5, and 6 post-activation of the PBMCs with the indicated antibody.

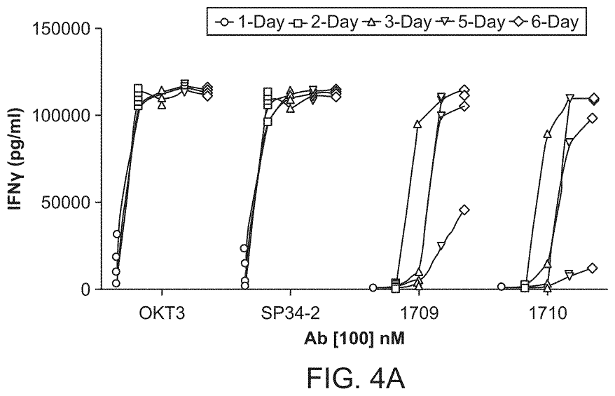

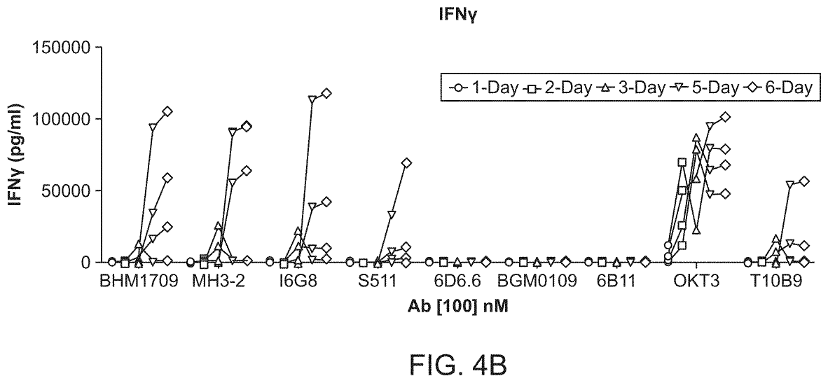

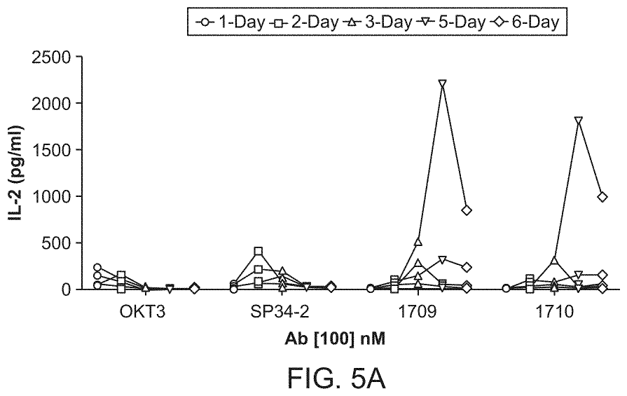

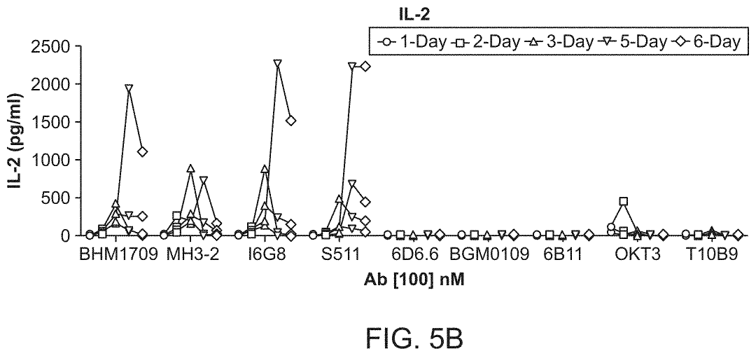

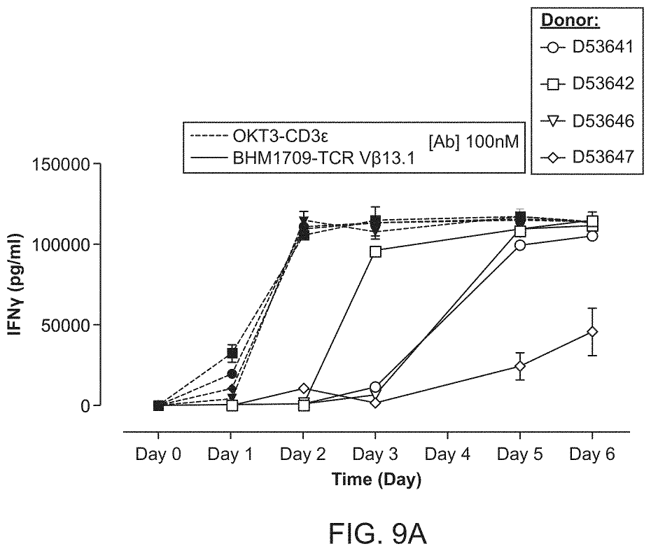

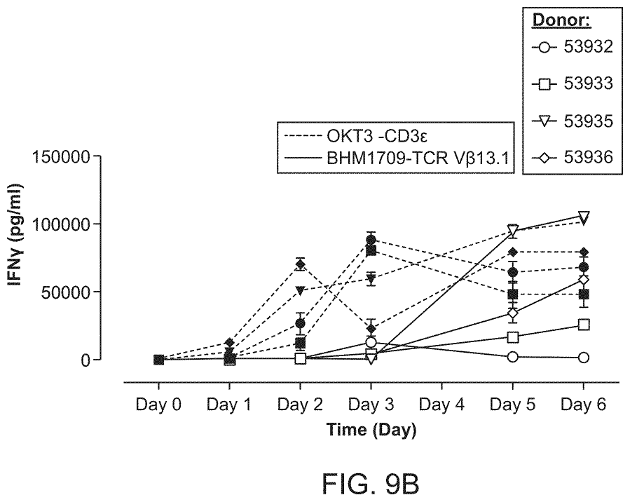

PBMCs activated using plate-bound BHM1709 or BHM1710 showed decreased secretion of IFNγ compared to PBMCs activated using anti-CD3ε antibodies (OKT3 or SP34-2) were used to activate human PBMCs (FIG. 4A and FIG. 5B). The kinetics of IFNγ production by anti-TCRβV antibody BHM1709-activated CD3+ T cells was delayed relative to those produced by CD3+ T cells activated by anti-CD3ε antibodies (OKT3 and SP34-2) (FIGS. 9A and 9B).

PBMCs activated with BHM1709 and BHM1710 resulted in increased IL-2 production (FIG. 5A) with delayed kinetics (FIG. 5B) as compared to PBMCs activated with anti-CD3ε antibodies (OKT3 or SP34-2). Anti-TCRβV antibodies activated PBMCs demonstrate peak production of IL-2 at Day 5 or Day 6 post-activation (FIG. 5B). In contrast, IL-2 production in PBMCs activated with OKT3 peaked at day 2 post-activation (FIG. 5B). As with IFNγ, the IL-2 effect (e.g., enhanced production of IL-2 and delayed kinetics) was similar across all anti-TCRβV antibodies tested (FIG. 5B).

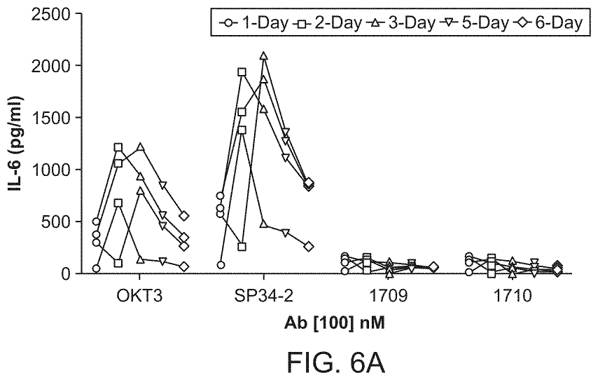

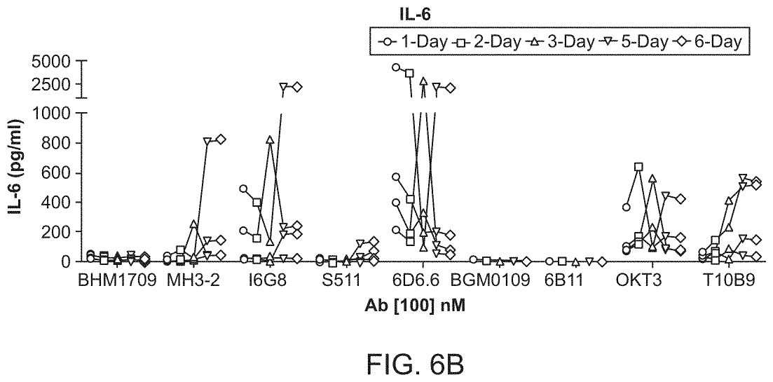

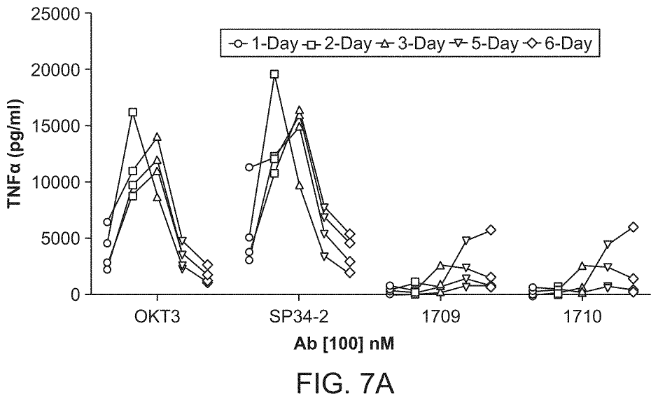

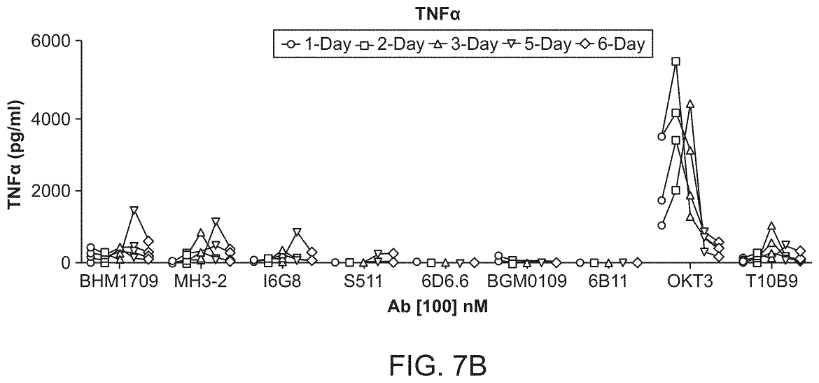

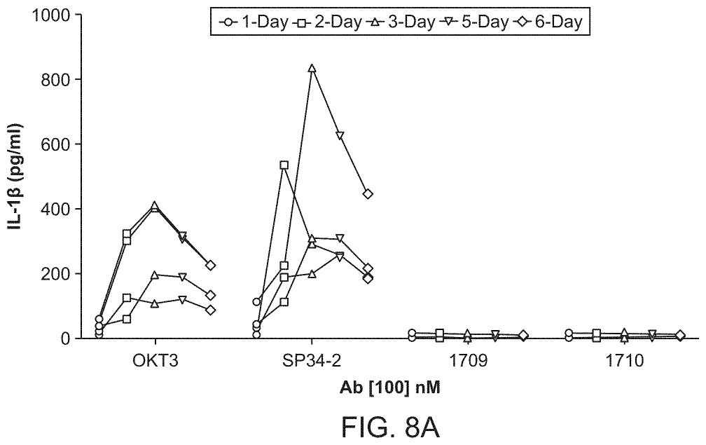

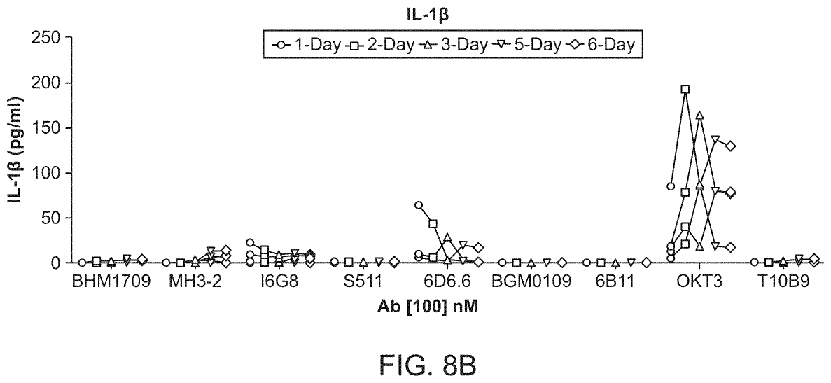

The production of cytokines IL-6, IL-1β and TNF-α which are associated with cytokine storms (e.g., CRS) were also assessed under similar conditions. FIGS. 6A, 7A, and 8A show that while PBMCs activated with anti-CD3ε antibodies demonstrate production of IL-6 (FIG. 6A), TNF-α (FIG. 7A) and IL-1β (FIG. 8A), no or little induction of these cytokines was observed with PBMCs activated with the anti-TCRβV antibodies BHM1709 or BHM1710. As shown in FIG. 7B and FIG. 8B, TNF-α and IL-1β production were not induced by activation of PBMCs with any of the anti-TCRβV antibodies.

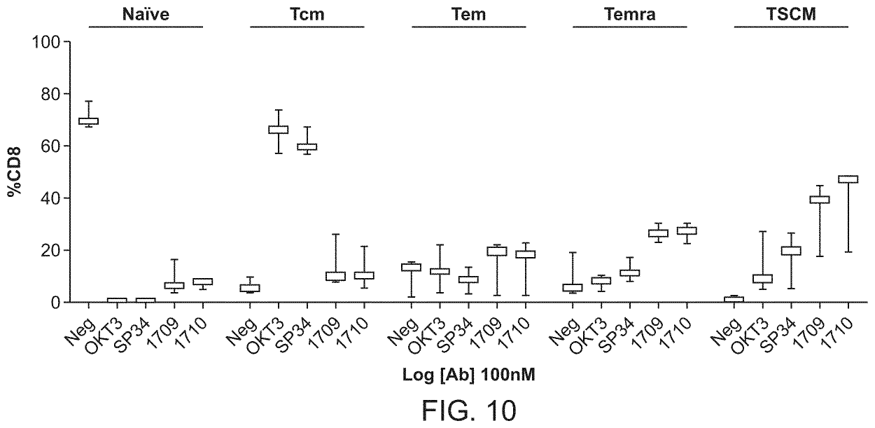

The subset of memory effector T cells known as TEMRA cells was preferentially expanded in CD8+ T cells activated by anti-TCRβV antibodies BHM1709 or BHM1710 (FIG. 10). Isolated human PBMCs were activated with immobilized (plate-coated) anti-CD3ε antibody or an anti-TCRβV 13.1 antibody at 100 nM for 6-days. After a 6-day incubation, T-cell subsets were identified by FACS staining for surface markers for Naive T cell (CD8+, CD95−, CD45RA+, CCR7+), T stem cell memory (TSCM; CD8+, CD95+, CD45RA+, CCR7+), T central memory (TCM; CD8+, CD95+, CD45RA−, CCR7+), T effector memory (TEM; CD8+, CD95+, CD45RA−, CCR7−), and T effector memory re-expressing CD45RA (i.e. TEMRA) (CD8+, CD95+, CD45RA+, CCR7−). Human PBMCs activated by anti-TCR Vβ13.1 antibodies (BHM1709 or BHM1710) increased CD8+ TSCM and TEMRA T cell subsets compared to PBMCs activated by anti-CD3e antibodies (OKT3 or SP34-2) (FIG. 10). Similar expansion was observed with CD4+ T cells.

The data provided in this Example shows that anti-TCRβV antibodies can preferentially activate a subset of T cells, leading to an expansion of TERMA cells. These cells can promote tumor cell lysis without inducing cytokine storm (e.g., cytokine release syndrome). Thus, ex vivo T cells (e.g., CAR T cells, TILs, T cells expressing an exogenous receptor (e.g., exogenous TCR)) can be activated and expanded using anti-TCRβV antibodies without or decreasing the severity of CRS when administered to a subject.

### Example 2: Reactivation of Anti-TCRβV Antibody-Activated and Expanded Purified T Cells In Vitro do not Induce CRS-Related Cytokines

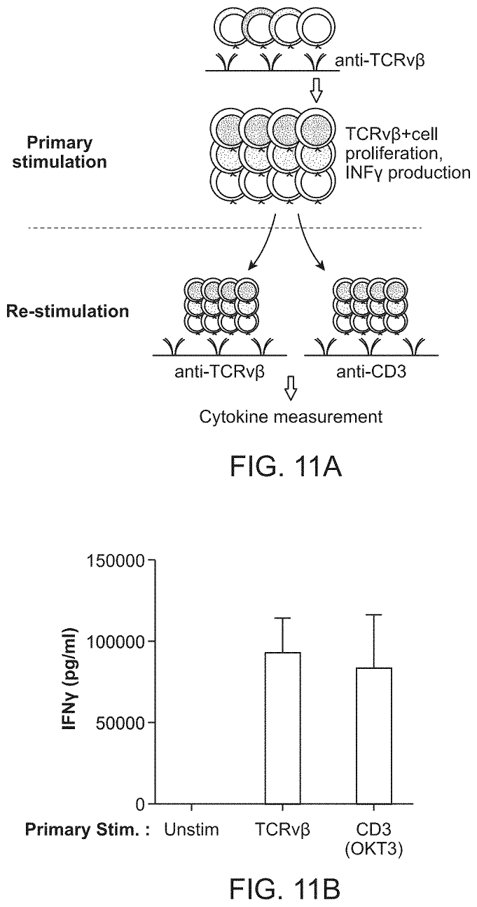

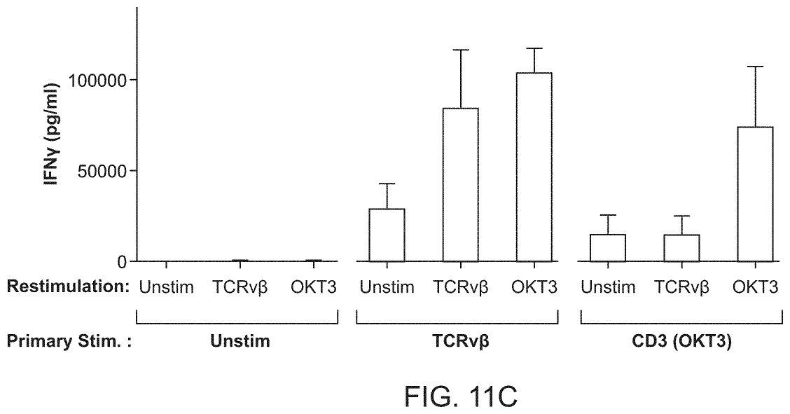

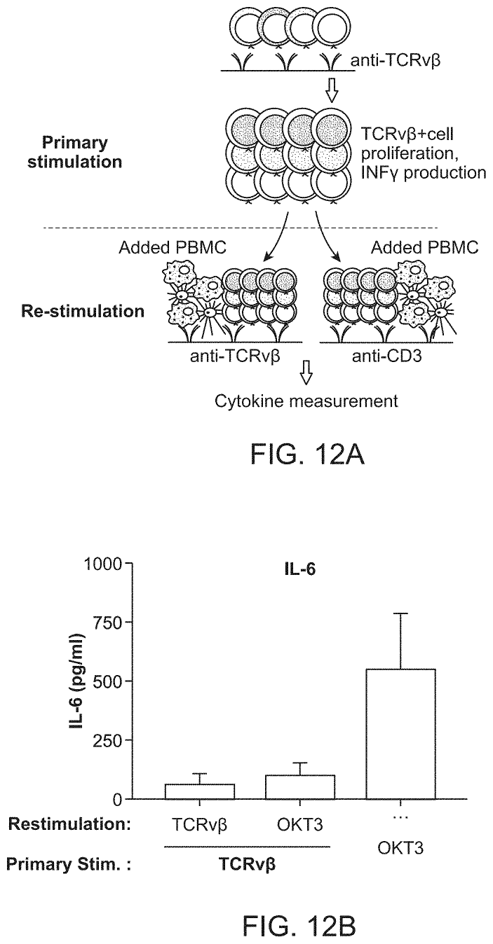

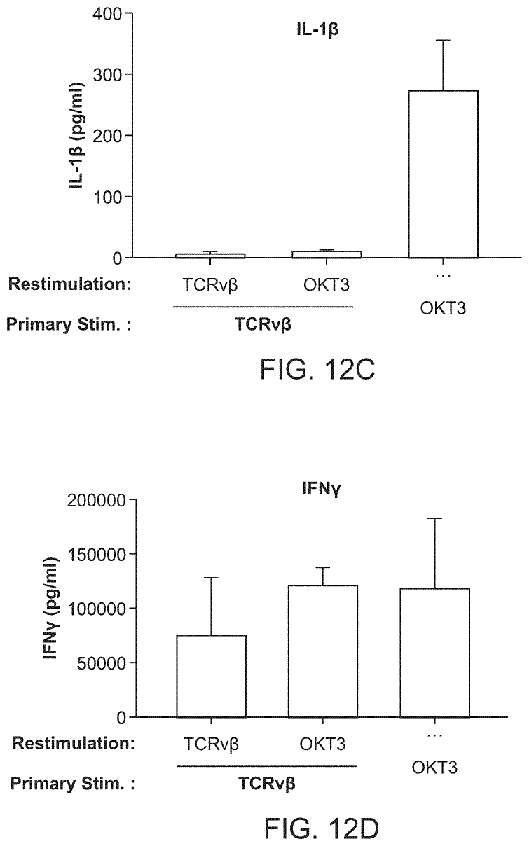

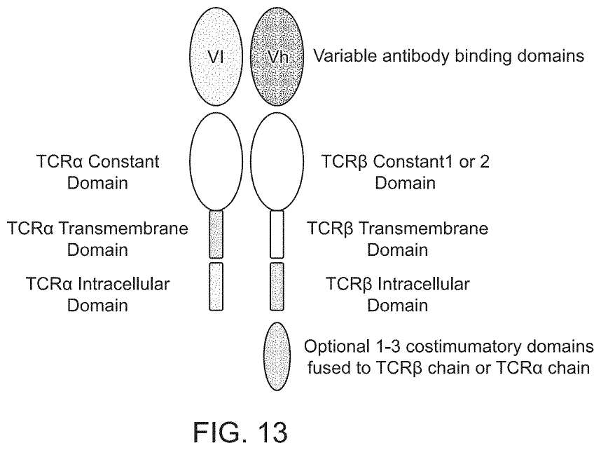

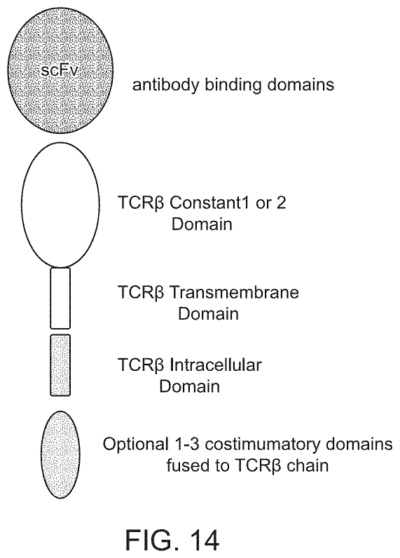

Healthy donor PBMCs or purified T cells were first activated ex vivo with anti-TCRβV antibody for 5 days with plate-bound antibodies. The anti-TCRβV antibody-activated and expanded cells were then stimulated for 2 days with fresh plate-bound anti-TCRβV antibodies or anti-CD3ε antibodies in the presence (FIG. 11A) or absence (FIG. 12A) of T cell depleted autologous PBMCs.

When plate-bound anti-TCRβV antibody was used to activate human PBMCs as a primary stimulation, the T cell cytokine IFNγ was induced (FIG. 11B). Following primary stimulation, plate-bound anti-TCRβV antibody or anti-CD3ε antibody (OKT3) were used to re-stimulate human PBMCs from the primary stimulation. Re-stimulation of anti-TCRβV antibody-activated T cells with anti-TCRβV antibodies resulted in higher IFNγ induction compared to anti-TCRβV antibody-activated T cells re-stimulated with anti-CD3ε antibodies (FIG. 11C).

When plate-bound anti-TCRβV antibody was used to activate human PBMCs as a primary stimulation followed by re-stimulation with plate-bound anti-TCRβV antibody, the T cells induced lower levels of IL-6 and IL-1β compared to human PBMCs activated with plate-bound anti-CD3ε antibody as a primary stimulation followed by re-stimulation with plate-bound anti-CD3ε antibody (FIG. 12B and FIG. 12C). Additionally, when plate-bound anti-TCRβV antibody was used to activate human PBMCs as a primary stimulation followed by re-stimulation with plate-bound anti-TCRβV antibody, the T cells induced IFNγ as did human PBMCs activated with plate-bound anti-TCRβV antibody as a primary stimulation followed by re-stimulation with plate-bound anti-CD3ε antibody (FIG. 12D). The data indicates that anti-TCRβV antibody-expanded T cells remain functionally active and do not induce CRS-related cytokines upon re-challenge with either anti-TCRβV antibodies or anti-CD3 antibodies.

### Example 3: Ex Vivo Expansion of Anti-TCRβV Antibody-Activated and Expanded CAR T Cells

For all conditions below activation antibodies were coated onto 24-well BD Falcon flat bottom plate at 100 nM (in PBS) for 2 hours at 37 degrees C. Subsequently, the plates were washed once with 500 μl fresh PBS prior to use. Three separate conditions tested included: Condition 1: equal amounts of TCRβV clonotype specific antibodies H131 and 16G8 (50 nM each in PBS); Condition 2: equal amounts of TCRβV clonotype specific antibodies H131 and 16G8 (50 nM each in PBS) and IL2 (culture medium containing 300 U/mL rIL-2 (cat. #Pr21269, ProMab); Condition 3: using equal amounts of anti-CD3ε and anti-CD28 antibodies (50 nM each in PBS).

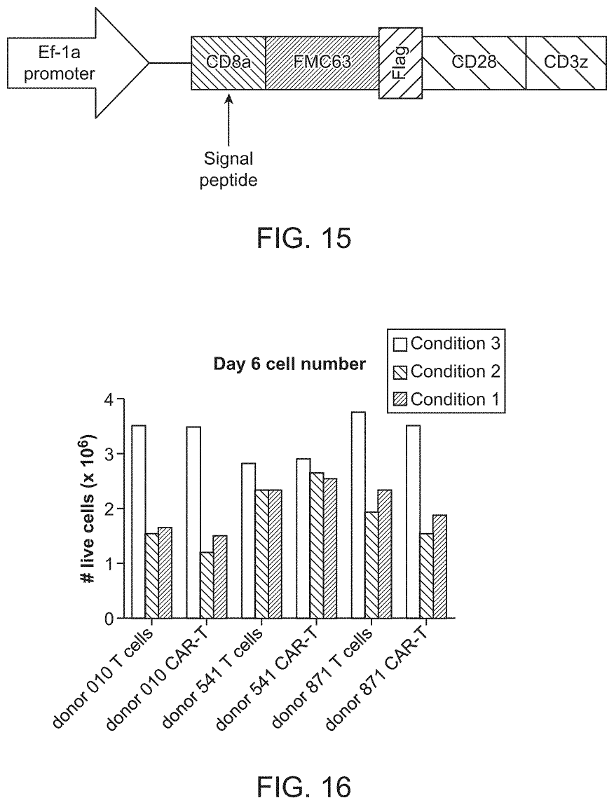

PBMCs from three individual healthy human donors were diluted at 1×10\hat{ }6 cells/ml in culture media (AIM V-AlbuMAXmedium (ThermoFisher) containing 10% FBS with 10 ng/ml IL-2 (ThermoFisher)). On day 1 of activation, 0.5 ml (0.5×10\hat{ }6/well) of PBMCs were seeded into the antibody coated 24-well plates (conditions described above) and incubated for 24 hours. On day 2 of activation, PBMCs were transduced with lentiviral particles containing an anti-CD19 chimeric antigen receptor (CAR) cassette (Promab cat. #PM-CAR1007, CD19SCFV-FLAG-CD28-CD3ζ) (FIG. 15). Prior to transduction, the culture medium was aspirated and replaced with fresh medium containing TransPlus transduction enhancer (1 μl of TransPlus, ProMab cat. #V050 in 500 μl culture medium). Lentivirus particles containing the CD19 CAR-T construct were added at MOI of 10:1 to each well and the plate rocked to mix well. Cells of all conditions were further expanded from this point in the presence of IL2 (culture medium containing 300 U/mL rIL-2 (cat. #Pr21269, ProMab). T cell expansion was continued for 9 days and samples collected to determine T cell count on days 5 and 9 post activation. Nine days post activation, the cells were collected and CAR-T expression analyzed by flow cytometry. A 16G8-PE labeled antibody was used to determine the increased percentage of clonotypic βV positive CAR-T cells. Total CAR expression (anti-CD19-Flag-CAR) on cells that had been transduced with the lentivirus was analyzed; and the ratio of CD4/CD8 T cells of CD3+ T cells was determined.

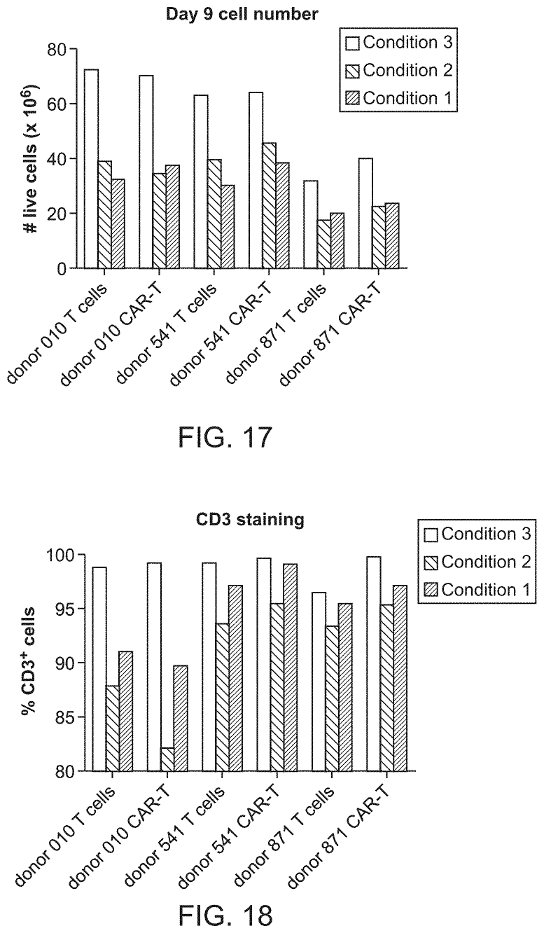

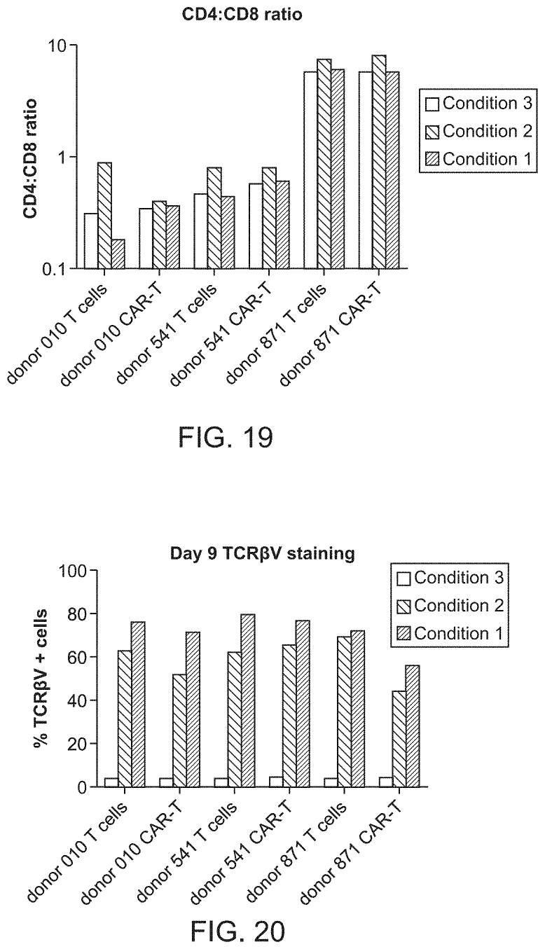

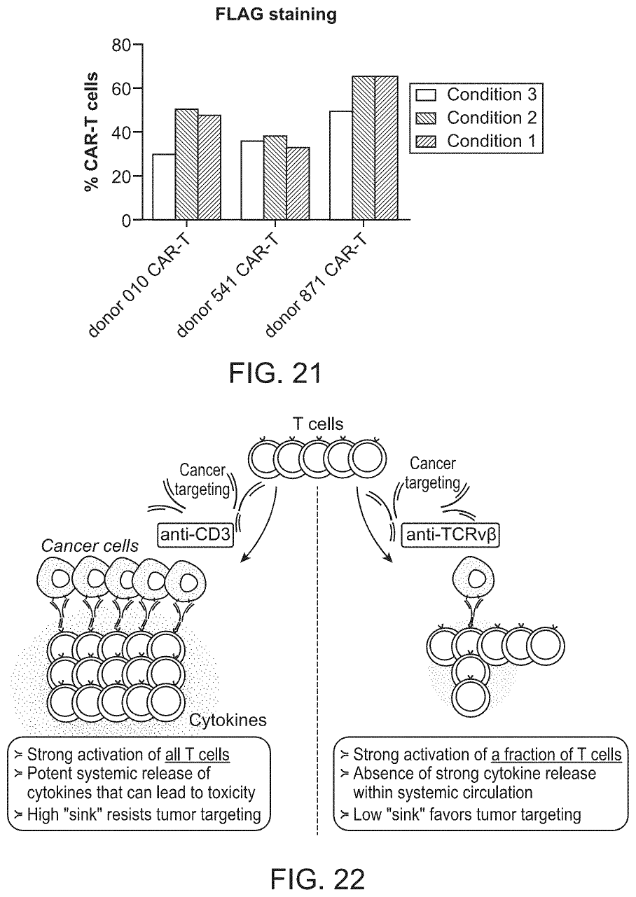

At 6 days (FIG. 16) and 9 days (FIG. 17) post activation, the number of live cells from Condition 2 and Condition 3 cultures were similar to or comparable to those of Condition 1. At 9 days post activation, the number of CD3+ T cells from Condition 2 and Condition 3 cultures were similar to or comparable to those of Condition 1 (FIG. 18). At 9 days post activation, the ratio of CD4:CD8+ T cells from Condition 2 and Condition 3 cultures were similar to or comparable to those of Condition 1 (FIG. 19). As described above, a 16G8-PE labeled antibody was used to determine the increased percentage of clonotypic βV positive CAR-T cells. As shown in FIG. 20, the percentage of clonotypic βV positive CAR-T cells increases in Condition 2 and Condition 3 as compared to Condition 1. As described above, the FLAG tag in the CAR construct was used to determine the percentage of T cells that expressed the CAR. The data presented in FIG. 21 shows that T cells activated under Condition 1 and Condition 2 contained about the same or greater percentages of T cells expressing the chimeric antigen receptor.

Nine days post activation the number of cells in each condition was analyzed (Table 11). As shown in Table 11, CAR-T cells expanded in the experimental Condition 1 and Condition 2.

TABLE 11
Cell counts 9 days post activation

| | Method of Activation | | |
|---|---|---|---|
| | Anti-CD3/CD28 | Anti-TCRβV | Anti-TCRβV |
| | antibodies | antibodies +IL2 | antibodies |
| | (Condition 1) | (Condition 2) | (Condition 3) |
| Cells | Cell Count (millions) | | |
| Donor 010 | 72 | 38.7 | 32.2 |
| T cells | | | |
| Donor 010 | 69.6 | 34.4 | 36.9 |
| CAR T cells | | | |
| Donor 541 | 62.4 | 39.4 | 29.6 |
| T cells | | | |
| Donor 541 | 63.6 | 45.3 | 38.4 |
| CAR T cells | | | |
| Donor 871 | 31.2 | 17 | 20 |
| T cells | | | |
| Donor 871 | 39.6 | 21.9 | 23.1 |
| CAR T cells | | | |

### Example 4: Kinetics of T Cell Expansion Following TCRβV 6-5 Stimulation

To assess the kinetics and absolute count of anti-TCRβv 6-5 expanded T cells—either PBMCs or purified T cells were stimulated with plate-immobilized anti-TCRvb 6-5 antibody over 8 days with a T cell-activating antibody at 100 nM. T cell activating antibodies tested included: i) anti-TCRvb 6-5 v1 antibody; ii) anti-TCRvb 6-5 v2; iii) OKT3 (anti-CD3ε antibody); iv) SP34-2 (anti-CD3ε antibody); and v) IgG1 N297A (isotype control). Cell pellets were collected each day and stained for CD3, CD4, CD8 and TCRvb 6-5 for flow analysis.

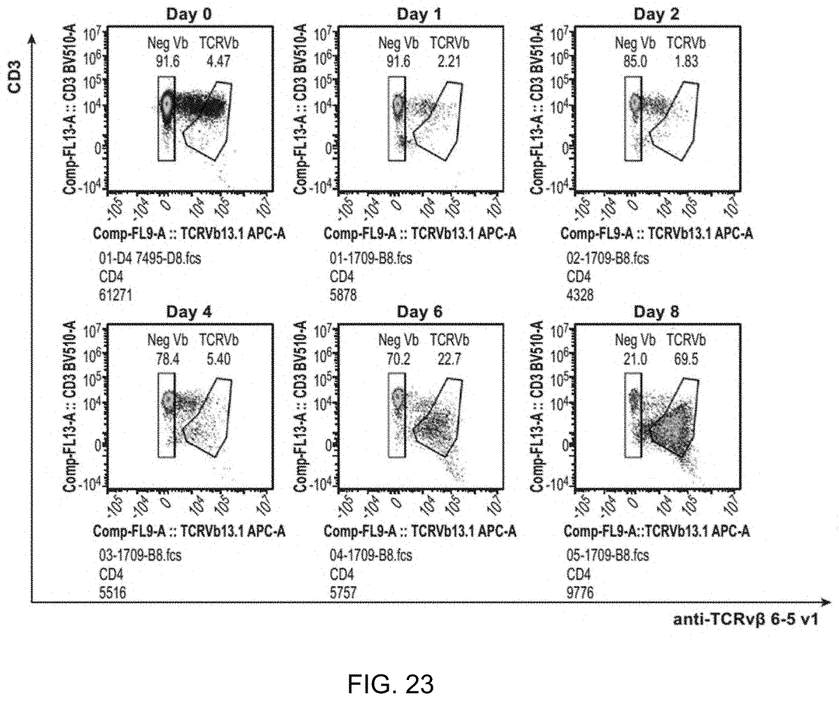

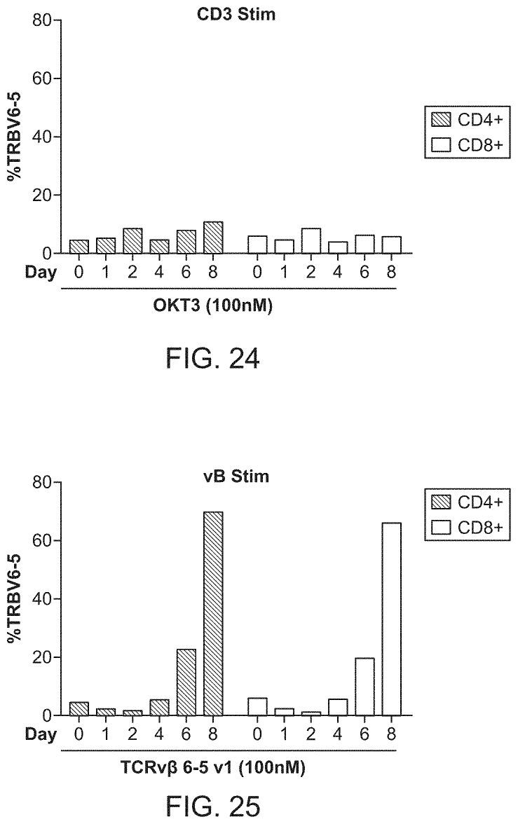

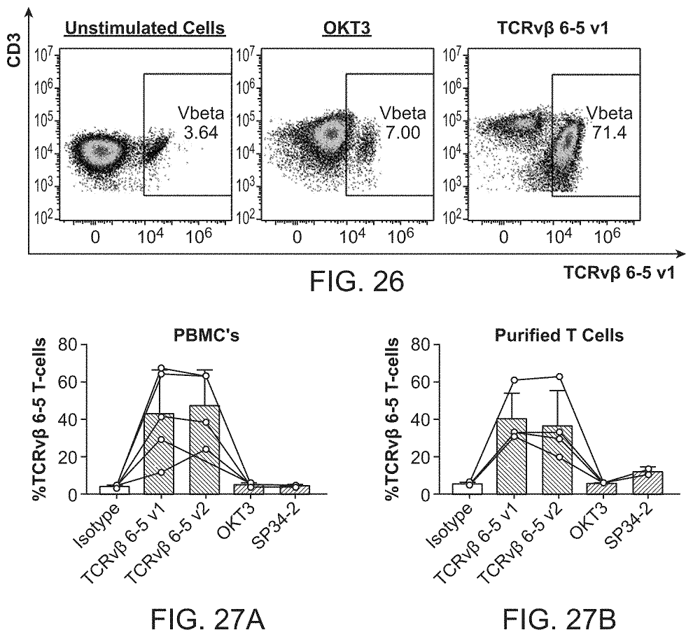

TCRvb 6-5+ T cell expansion over 8 days using anti-TCRvb 6-5 v1 is shown in FIG. 23, as assessed by flow cytometry. The data is for a single representative donor; and similar results were seen with PBMCs from two other independent donors. FIG. 25 further shows the specific expansion of TCRvb 6-5+ CD4+ T cells and TCRvb 6-5+ CD8+ T cells by TCRvb 6-5 vl. In contrast, there was no specific TCRvb 6-5+ T-cell expansion by OKT3 (FIG. 24; FIG. 26). FIGS. 27A and 27B show selective expansion of TCRβV 6-5+ T cells in human PBMCs (FIG. 27A) and purified T cells (FIG. 27B)

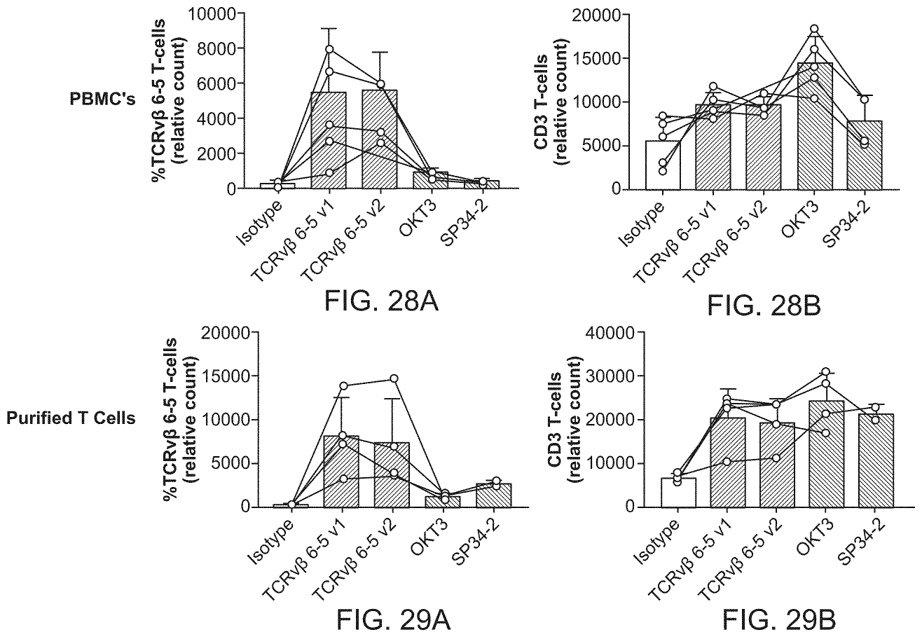

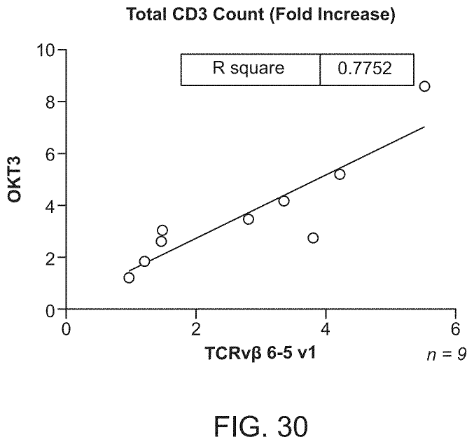

FIGS. 28A-30 shows that anti-TCRβV and anti-CD3ε antibodies expand T cells in a PBMC culture (FIGS. 28A and 28B) or a purified T cell culture (FIGS. 29A and 29B)) to comparable levels after 8 days, as measured by both relative count of TCRVB 6-5+ T cells (FIGS. 28A-29B) and relative count of total CD3+ T cells (FIGS. 28A-30).

### Example 5: Activated TCRvb 6-5+ T Cells Exert Cytolytic Function

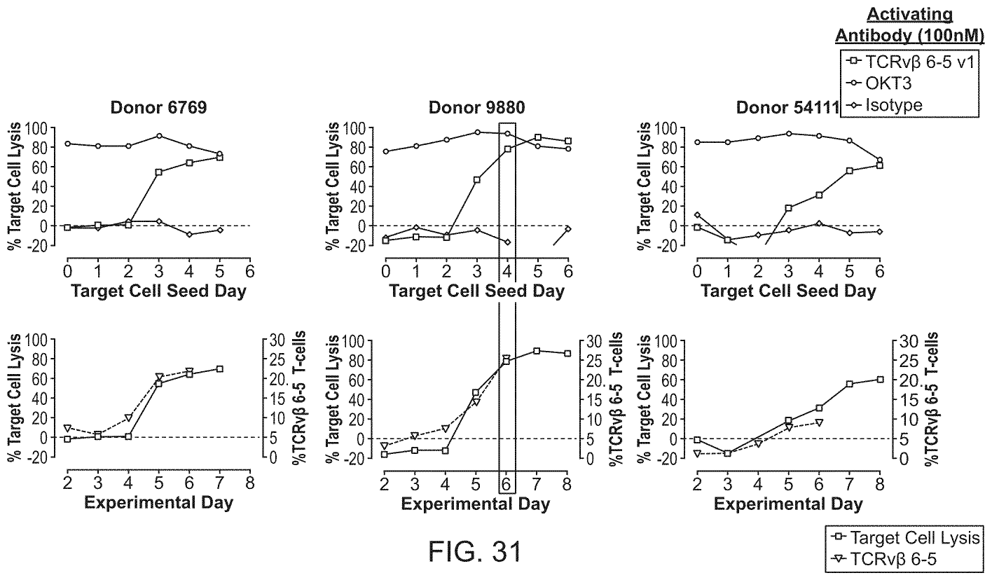

To assess the ability of T cells activated/expanded with anti-TCRVβ to mediate tumor cell lysis purified T cells were stimulated over 6 days with an immobilized T cell-activating antibody at 100 nM. T cell activating antibodies tested included: i) TCRvb 6-5 vl antibody; ii) OKT3 (anti-CD3ε antibody); or iii) IgG1 N297A (isotype control). Target cells (RPMI-8226 cells) were added on each day and incubated with the activated T cells at an initial effector T cell:target (E:T) cell ratio of 5:1 for 48 hours. Quantification of target cell lysis was measured using CFSE/CD138 and DRAQ7 FACS staining. Three different T cell donors were used (donor 6769, donor 9880, donor 54111). The data shows that the kinetics of target cell lysis by TCRVb 6-5 v1 activated T cells correlates with the expansion of TCRvb 6-5+ T cells (FIG. 31).

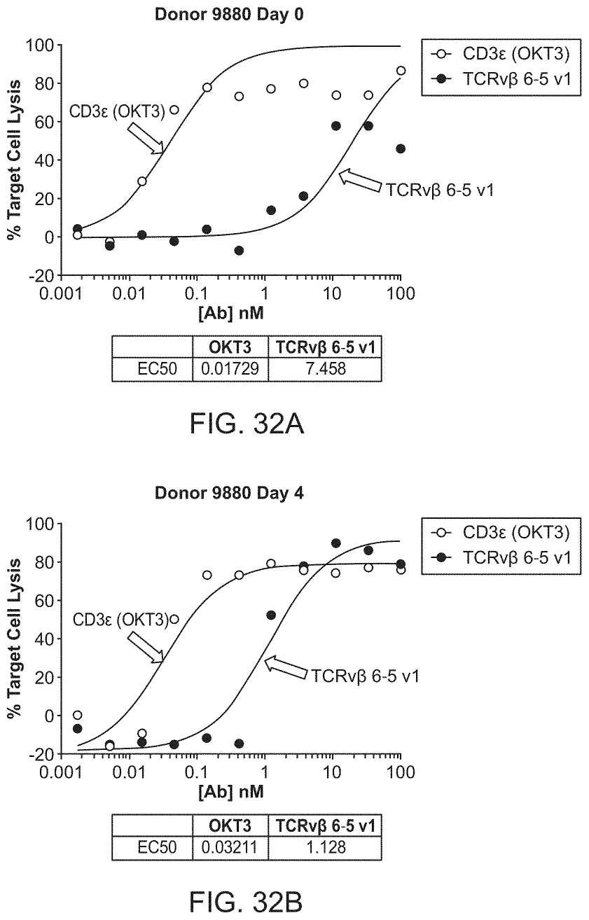

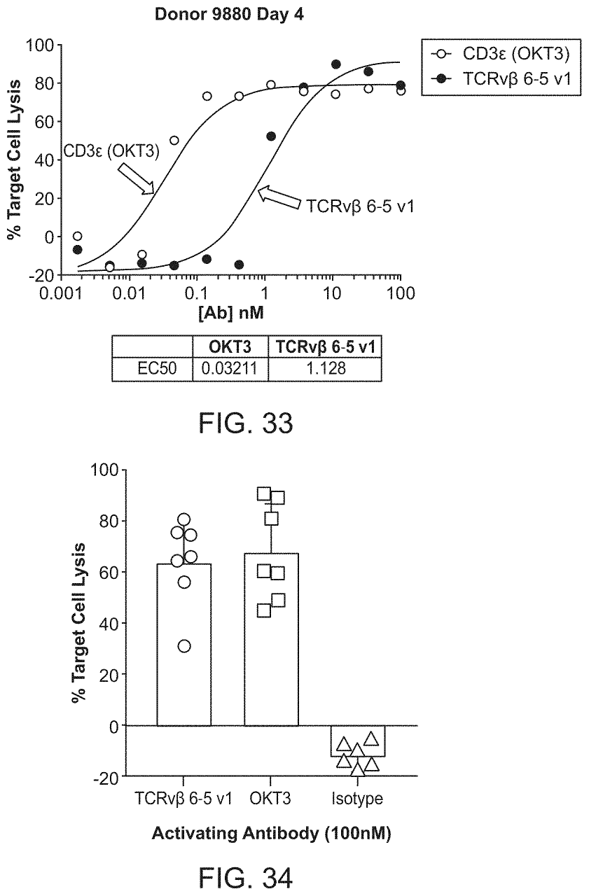

To further assess target cell lysis OKT3 or TCRvb 6-5 v1 antibodies were immobilized (plate-coated) with a ½ log serial dilution from a top dose concentration of 100 nM for purified T-cell (pan CD3 isolated) activation. The purified T-cells were stimulated with the activation plate for 0 (i.e. without antibody preactivation) to 4 (i.e. with antibody preactivation) days prior to addition of the target cells. Target cells (RPMI8226) were added to the activation plate (at an initial E:T cell ratio, 5:1) for up to 6-days (i.e. for plate 0, E:T coculture for 6-days, and for plate 4, E:T coculture for 2-days) followed by target cell lysis quantification via CFSE/CD138 and DRAQ7 FACS staining. The data shows that without T-cell preactivation, approximately 3% of Vb cells were able to kill target cells at day 6 (at higher concentration) (FIG. 32A); and with T-cell preactivation, approximately 25% of Vb cells were able to kill target cells at day 6 (the killing curve is shifted to the left) (FIG. 32B). TCRvb 6-5 v1 activated T cells exhibit comparable maximal target cell lysis when compared to anti-CD3ε when T cells are preactivated for 4 days (FIG. 33). At 100 nM, TCRvb 6-5 v1 activation shows comparable killing of target cells to anti-CD3ε activation (FIG. 34) (preactivation between 4-6 days depending on the donor and the cultures cultured for 48 h in presence of target cells).

### Example 6: Assessing TCRvb Downregulation/Internalization by Anti-TCRvb 6-5 Antibody

To assess the effect anti-TCRvb 6-5 mediated T cell activation has on cell surface expression of TCRvb—purified T cells were stimulated over 8 days with the indicated T cell-activating antibody at 100 nM (plate bound). T cell activating antibodies included: i) anti-TCRvb 6-5 v1 antibody; or ii) SP34-2 (anti-CD3ε antibody). Cell pellets were collected each day and stained for CD3, CD4, CD8 and TCRβV 6-5 for flow cytometry analysis. A total of three donors were tested, each showing similar results.

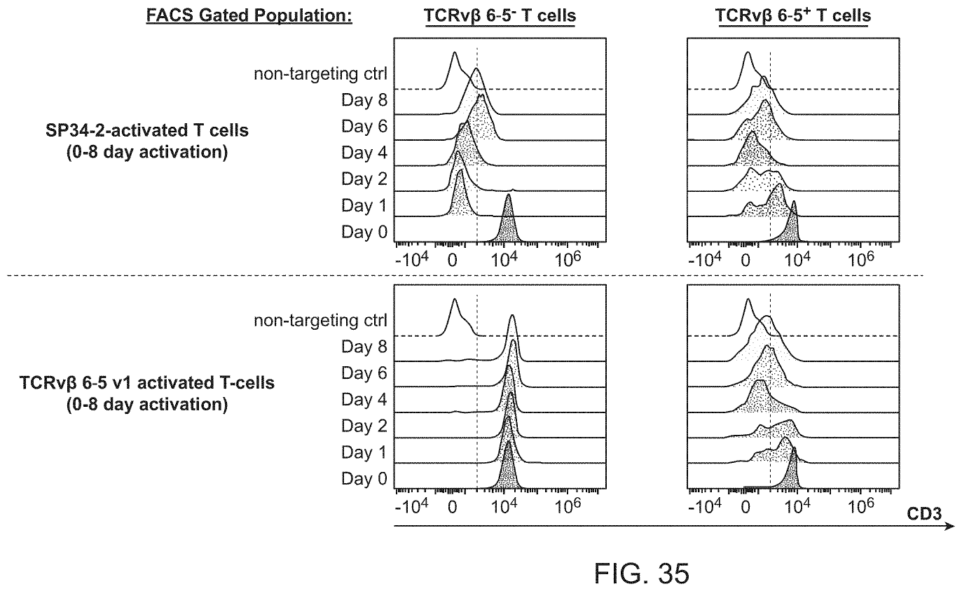

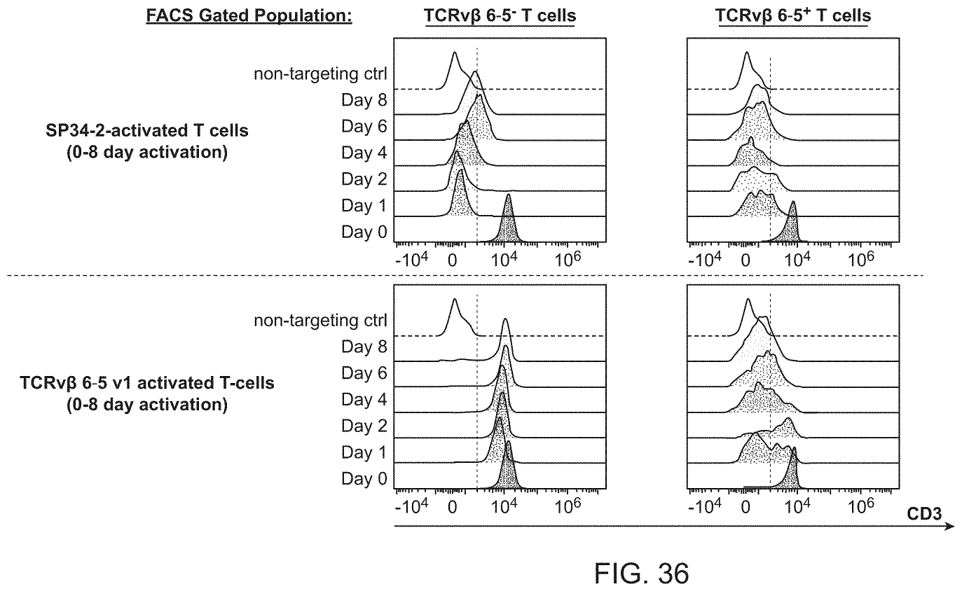

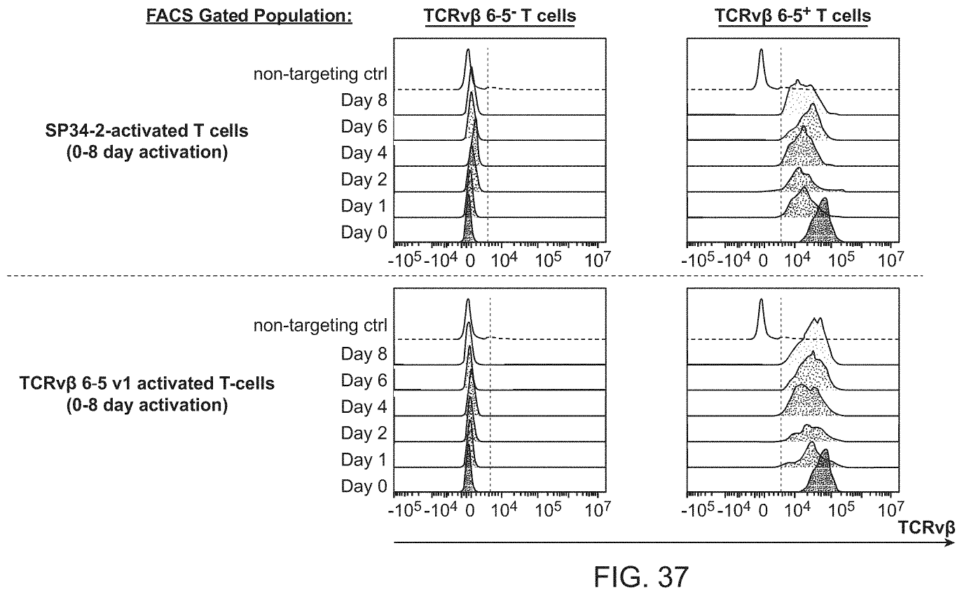

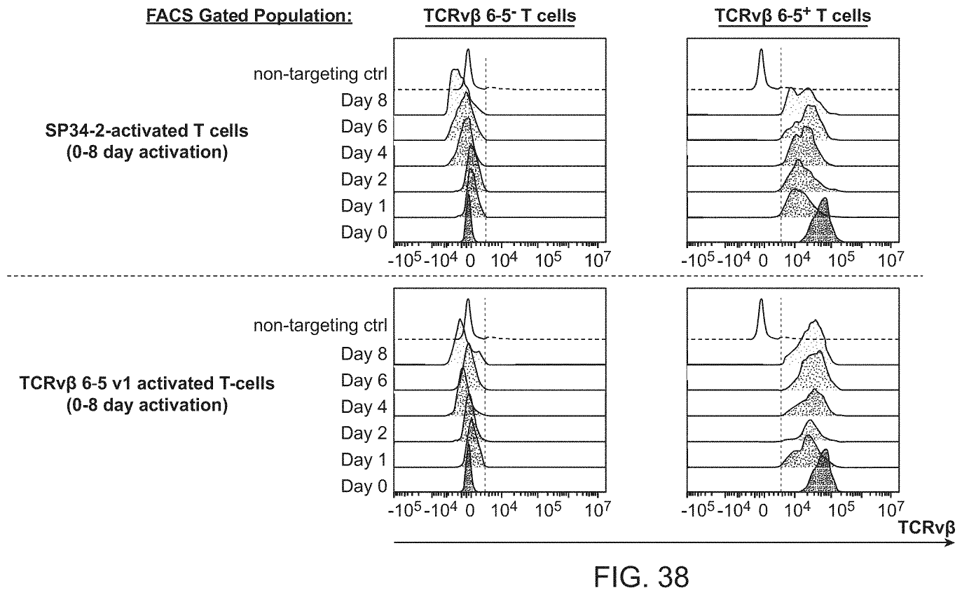

The results show that both anti-CD3ε and anti-TCRvb antibodies activated CD4+ T cells (FIG. 35) and activated CD8+ T cells (FIG. 36) display reduced CD3ε cell surface expression; whereas, TCRvb 6-5 cell surface expression on CD4+ T cells (FIG. 37) and CD8+ T cells (FIG. 38) remains detectable post T cell activation. The results show that the CD3ε subunit is downregulated/internalized in T cells activated by either anti-CD3ε or anti-TCRvb antibodies; while TCRvb 6-5 remains detectable post T cell activation. Additionally, CD4 and CD8 staining did not show any signs of downmodulation of these receptors by either antibody.

### Example 7: Cynomolgus Cross Reactivity of Anti-TCRβV Antibodies

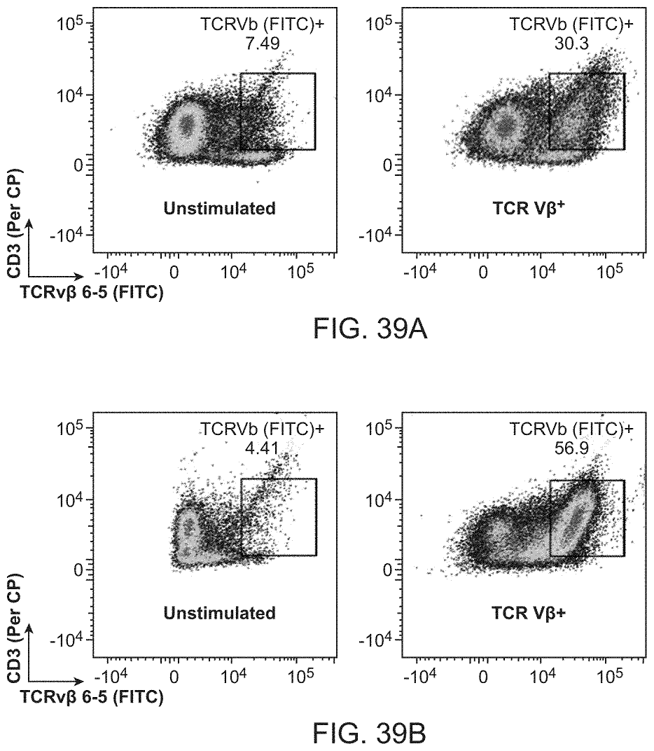

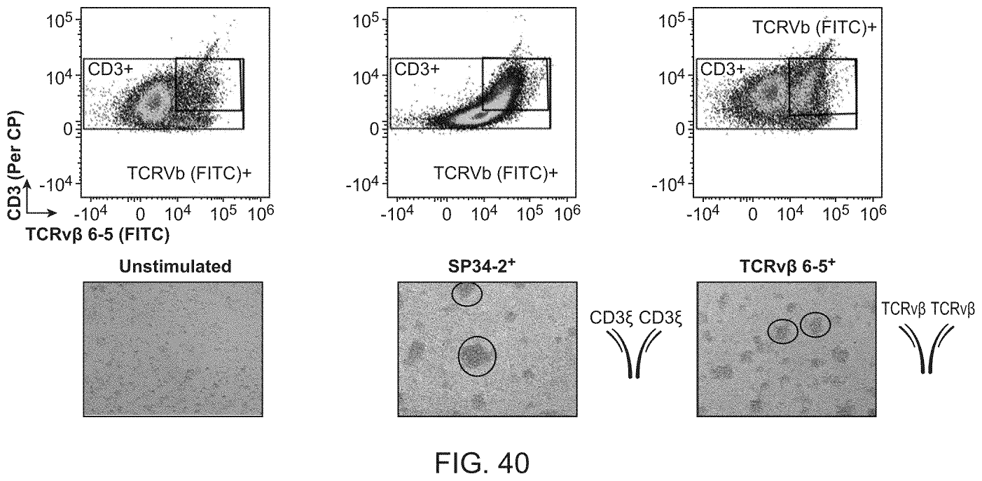

To assess the cross reactivity of anti-TCRβV antibodies for cynomolgus TCRβV clonotype fresh and cryopreserved cynomolgus PBMCs were cultured in complete media (RPMI with 10% FBS) in tissue culture treated flat bottom 96 well plates precoated with anti-TCRβV 6-5 v1 or anti-CD3ζ antibodies at 100 nM concentration. Negative control or unstimulated wells received PBS alone. TCRβV 6-5 expression was evaluated after 6 days in culture using CytoFlex flow cytometer (Beckmann Coulter) and imaged. Two donors samples were used: Donor DW8N—fresh PBMC sample, male, age 8, weight 7.9 kgs (data presented in FIG. 39A); Donor G709—cryopreserved sample, male, age 6, weight 4.7 kgs (data presented in FIG. 39B). The data show that cynomolgus T cells were activated and expanded by the anti-TCRβV 6-5 v1 (FIG. 39A and FIG. 39B). Fresh cynomolgus PBMCs from donor DW8N that had shown TCRvb 6-5 expansion were cryopreserved and after a week in cryopreservation, the cells were thawed and stimulated using anti-CD3 and anti-TCRvb 6-5 v1 for seven days. Cluster formation and expansion were both reproducible as shown in FIG. 40.

### Example 8: No Activation of γδ T Cells by Anti-TCRβV Antibodies

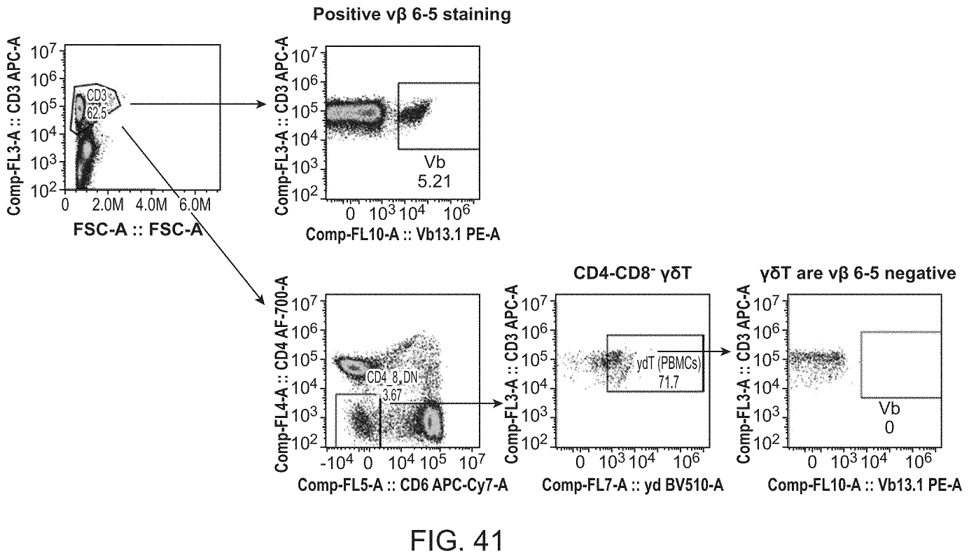

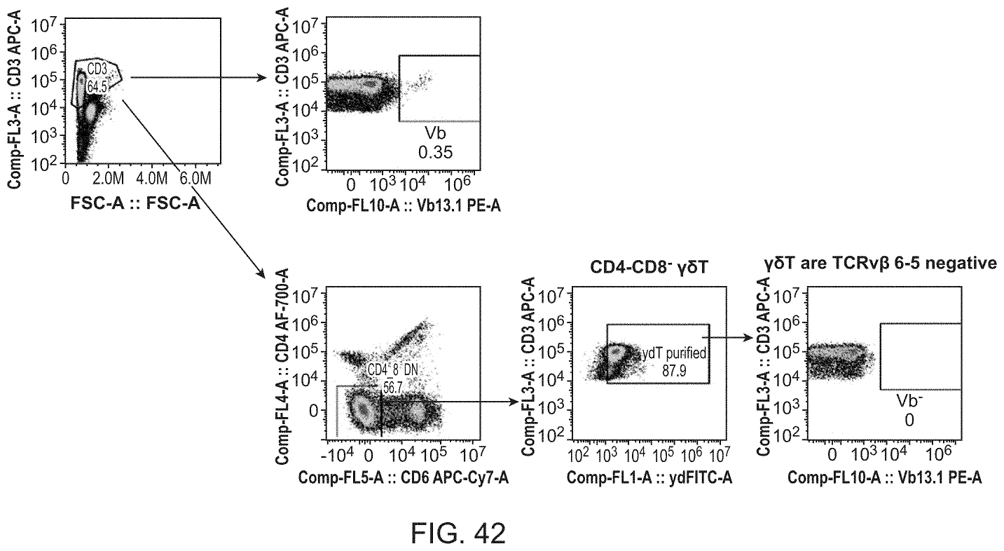

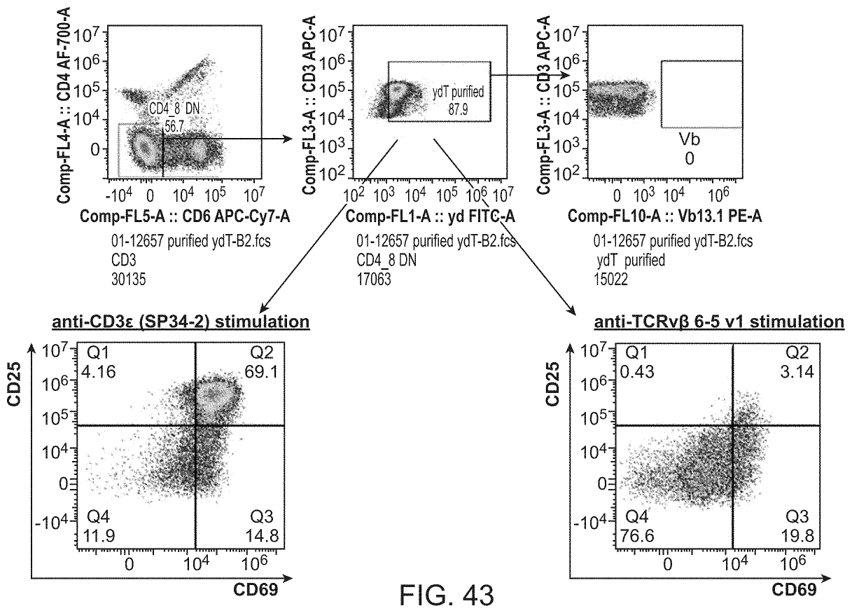

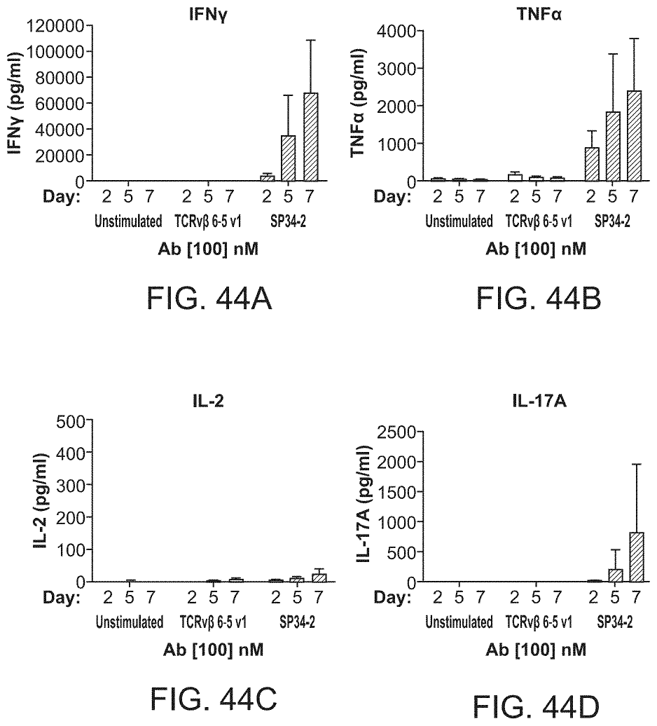

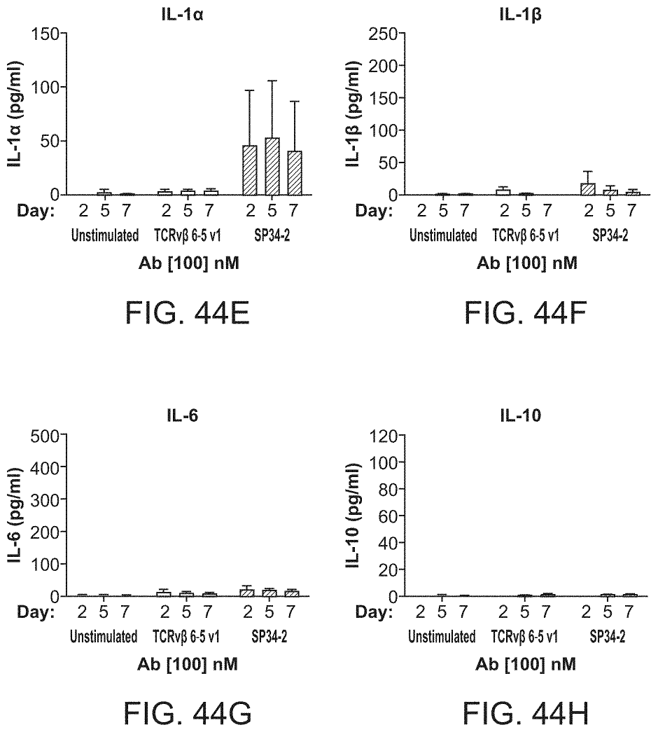

To determine if anti-TCRvb antibodies are able to activate γδ T cells-γδ T cells were purified from human PBMCs via magnetic bead separation. γδ T cells were immobilized on plate-coated anti-CD3ε (SP34-2) or anti-TCRvb 6-5 (anti-TCRvb 6-5 v1) antibodies for 24 hours and analyzed for CD69 and CD25 expression by flow cytometry. Supernatants were collected post activation 2, 5, and 7 days, and analyzed for cytokines using Meso Scale Discovery (MSD) assay. FACS gating/staining on PBMCs was conducted prior to γδ T cell purification showing that γδ T cells are vβ6-5 negative (Donor 12657-gating for γδ T and TCRvβ 6-5 based on FMO) (FIG. 41). FACS gating/staining on purified γδ T cell was conducted showing that purified γδ T cells are vβ 6-5 negative (Donor 12657—gating for γδ T and TCRvβ 6-5 based on FMO) (FIG. 42). As shown in FIG. 43, the anti-TCR Vβ 6-5 antibody (anti-TCRvb 6-5 v1) did not activate γδ T cells; while the anti-CD3ε antibody (SP34-2) did. The cytokine analysis showed that anti-TCRβV 6-5 v1 does not induce cytokine release by γδ T cells, cytokines analyzed include IFNγ, TNFα, IL-2, IL-17A, IL-1α, IL-1β, IL-6, and IL-10 (FIG. 44A-44H).

### Example 9: Polyclonal T Cell Expansion by Anti-TCRVβ Antibodies

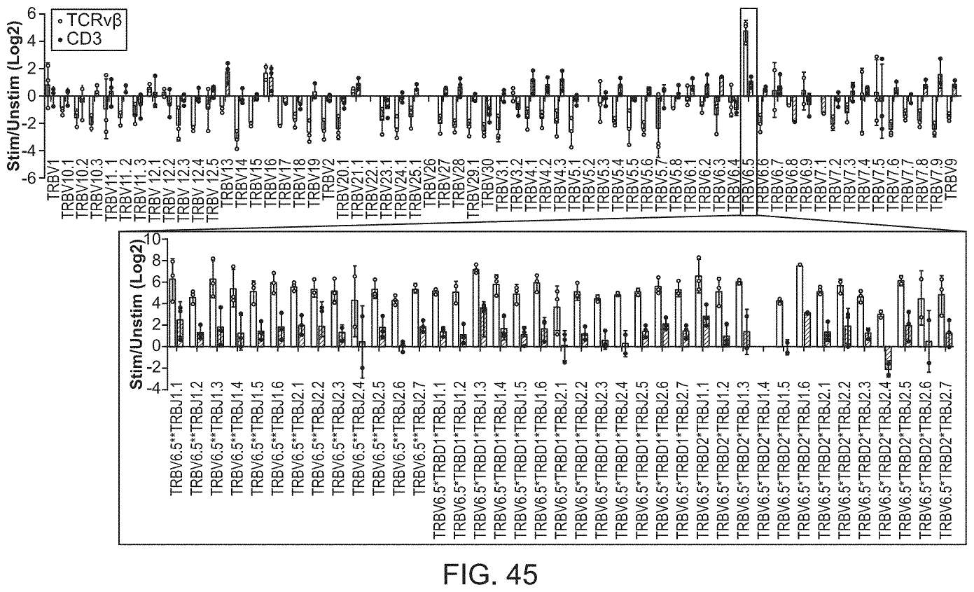

To assess the ability of anti-TCRVβ antibodies to induce polyclonal T cell expansion—human CD3+ T cells were isolated using magnetic-bead separation (negative selection) and activated with immobilized (plate-coated) anti-TCRβV 6-5 v1 at 100 nM for 6 days. The expanded T cell population was washed and lysed using Takara single cell lysis buffer for SMART(er) TCR cDNA synthesis and sequencing. TCR sequencing was carried out and absolute counts and relative representation of the different TCR alpha V and J segments and TCR beta V, D, and J segments were determined, as well as the different variants of each of them that arise from Artemis/TdT activity during the V(D)J recombination, and that correspond to unique clones of T cells. FIG. 45 shows the relative representations of all TCR alpha V segments (TRAV group of genes) and their variants (top), all TCR beta V segment 6-5 variants (TRBV6-5 gene) (bottom left), and all TCR beta V segments and variants excluding 6-5 (bottom right). The data show that the anti-TCRVβ antibody stimulation does not induce proliferation of specific T cell clones within the TRBV6-5 positive population, as the relative difference in clonal representation in that population is comparable to the TRBV6-5 negative population as well as total TRAV usage.

### Example 10: T Cells Expanded by Anti-TCRβV Represent a Novel Subset of Recently Activated Effector T Cells

To assess the phenotype of anti-TCRβV expanded T cells purified T cells were stimulated with solid-phase anti-TCRβV antibody over 8 days with the indicated T cell-activating antibody at 100 nM: i) anti-TCRvb 6-5 v1 antibody; ii) anti-TCRvb 6-5 v2; iii) OKT3 (anti-CD3ε antibody); or iv) IgG1 N297A (isotype control). T-cell subsets were identified by FACS staining for specific surface markers for: Naive T cell (CD4/CD8+, CD45RA+, CCR7+); T stem cell memory (TSCM; CD4/CD8+, CD95+, CD45RA+, CCR7+); T central memory (TCM; CD4/CD8+, CD95+, CD45RA−, CCR7+); T effector memory (TEM; CD4/CD8+, CD95+, CD45RA−, CCR7−); T effector memory re-expressing CD45RA (TEMRA; CD4/CD8+, CD95+, CD45RA+, CCR7−); and CD27, CD28, 4-1BB, OX40, and ICOS. Data is representative of more than 5 independent experiments.

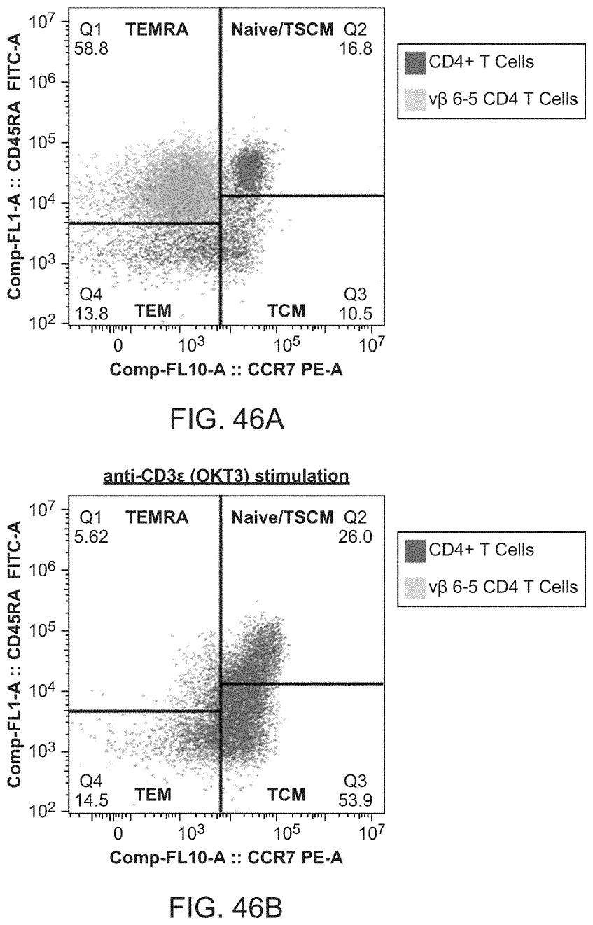

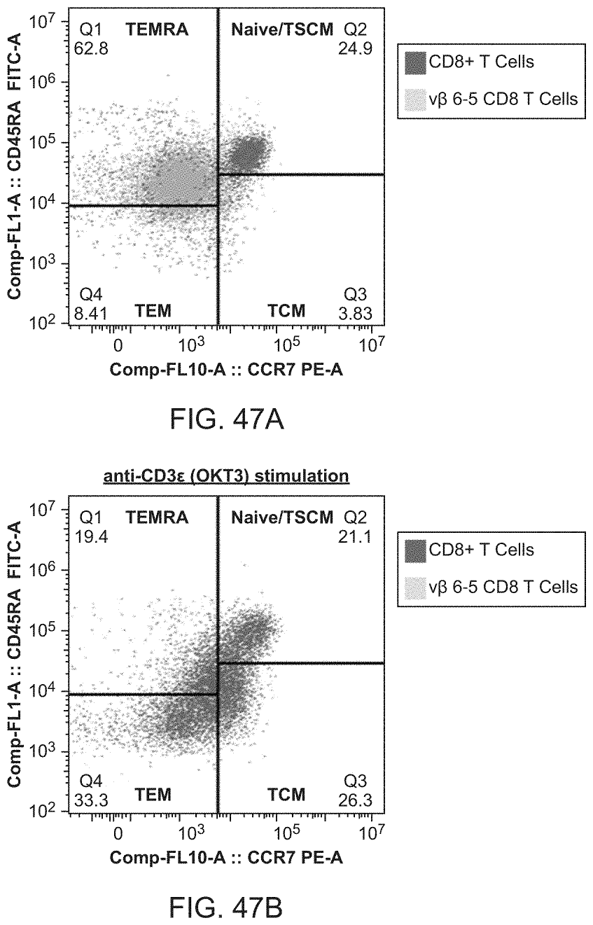

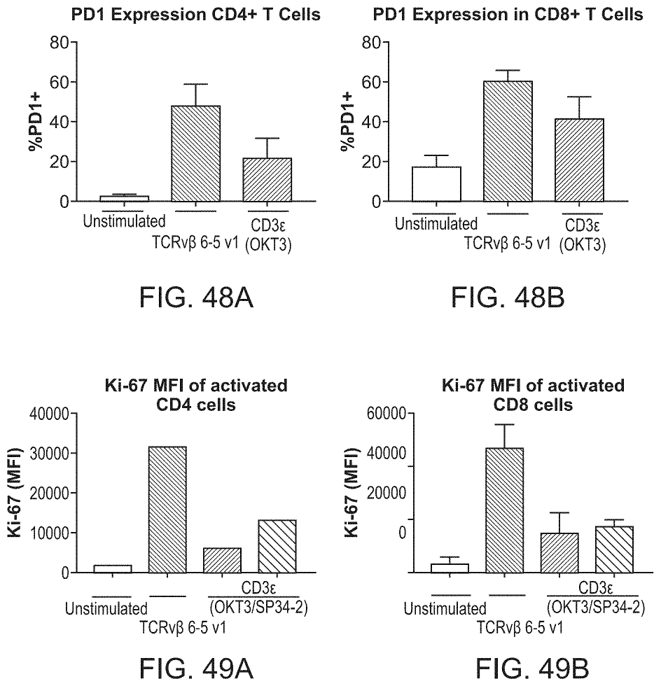

The data shows that CD4+ T cells expanded by anti-TCR Vβ antibody (FIG. 46A), but not OKT3 (FIG. 46B), share phenotypic markers with the TEMRA subset. Likewise, the data shows that CD4+ T cells expanded by anti-TCR Vβ antibody (FIG. 47A), but not OKT3 (FIG. 47B), share phenotypic markers with the TEMRA subset. Further analysis of PD1 expression showed anti-TCR Vβ activated CD4+ T cells (FIG. 48A) and CD8+ T cells (FIG. 48B) display increased PD1 expression relative to anti-CD3ε activated CD4+ T cells (FIG. 48A) and CD8+ T cells (FIG. 48B). These anti-TCR Vβ activated CD4+ T cells (FIG. 49A) (PD-1+ TEMRA phenotype) and anti-TCR Vβ activated CD8+ T cells (FIG. 49B) (PD-1+ TEMRA phenotype) show Ki-67 enriched phenotype relative to anti-CD3ε activated CD4+ T cells (FIG. 49A) and CD8+ T cells (FIG. 49B).

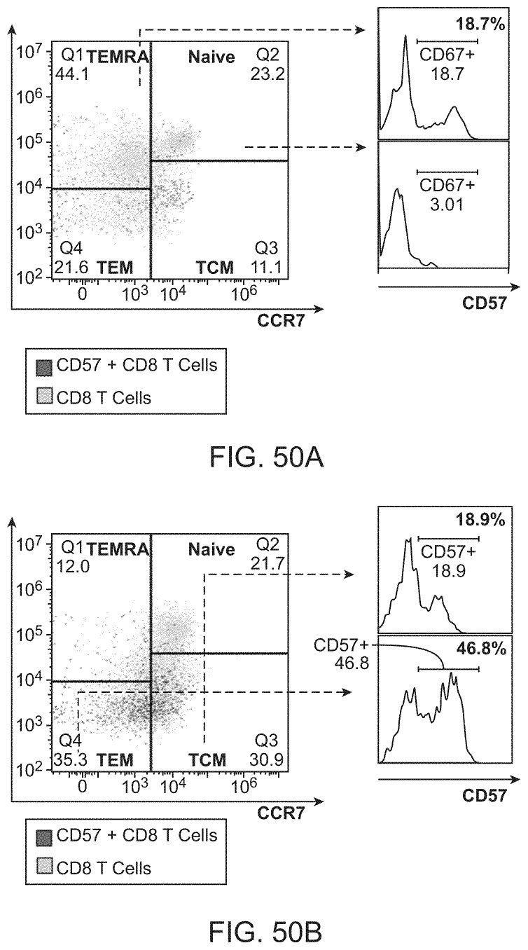

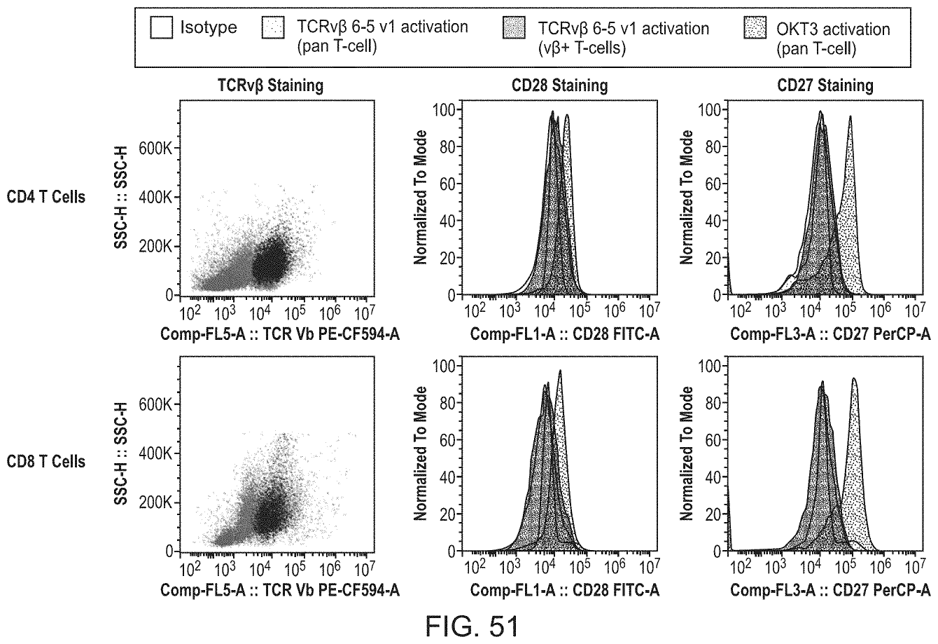

Further analysis of CD57 expression showed anti-TCR Vβ activated CD8+ T cells (FIG. 50A) do not display increased CD57 expression relative to anti-CD3ε activated CD8+ T cells (FIG. 50B). Likewise, analysis of CD27 and CD28 expression showed anti-TCR Vβ activated CD4+ T cells (FIG. 51 top) and anti-TCR Vβ activated CD8+ T cells (FIG. 51 bottom) do not display increased CD27 and CD28 expression relative to anti-CD3ε activated CD8+ T cells (FIG. 51).

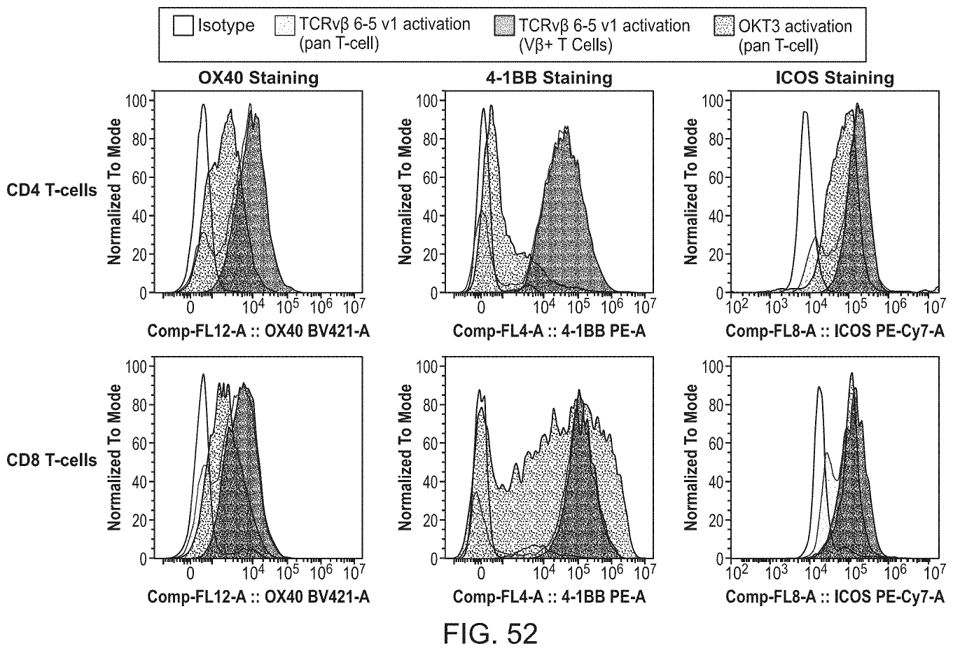

Further analysis of OX40, 41BB, and ICOS expression showed anti-TCR Vβ activated CD4+ T cells (FIG. 52 top) and anti-TCR Vβ activated CD8+ T cells (FIG. 52 bottom) display increased OX40, 41BB, and ICOS expression relative to anti-CD3ε activated CD8+ T cells (FIG. 52).

The TEMRA like phenotype of anti-TCR Vβ antibody expanded T cells was further analyzed using time lapse flow cytometry to evaluate expression of CD45RA and CCR7 at different time points post activation. Isolated human T-cells were activated with immobilized (plate-coated) anti-CD3ε or anti-TCR Vβ at 100 nM for between 1-8-days. After each (1, 2, 3, 4, 5, 6, 8−) day activation, T-cell subsets were identified by FACS staining for surface markers for Naïve/TSCM T cell (CD4+/CD8+, CD45RA+, CCR7+), T central memory (TCM; CD4+/CD8+, CD95+, CD45RA−, CCR7+), T effector memory (TEM; CD4+/CD8+, CD95+, CD45RA−, CCR7−), and T effector memory re-expressing CD45RA (TEMRA; CD4+/CD8+, CD95+, CD45RA+, CCR7−). TCRβV+ T-cells are identified by TCR Vβ+ staining. FACS stained samples were analyzed by flow cytometry analysis. Data shown a representative for CD4+ T-cells from 1 of 3 donors.

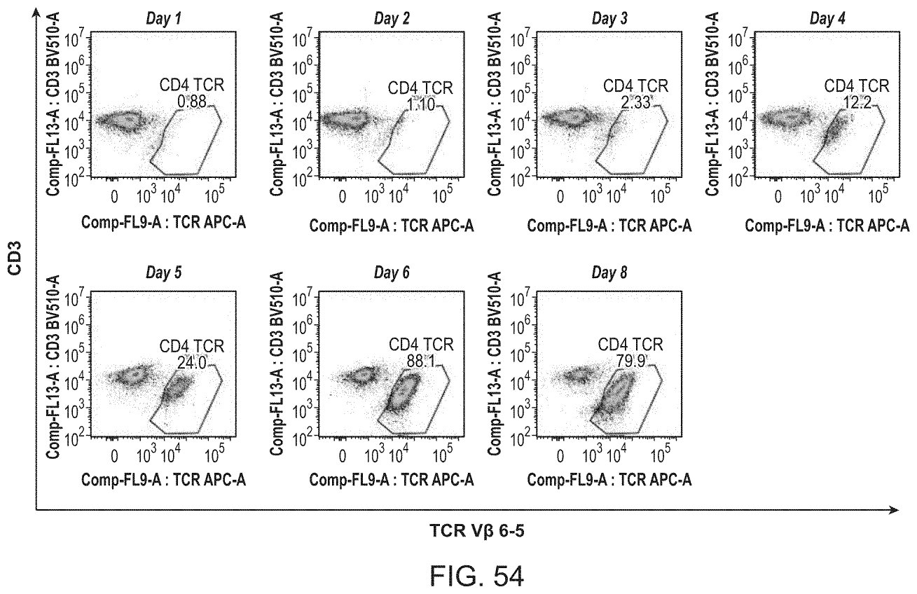

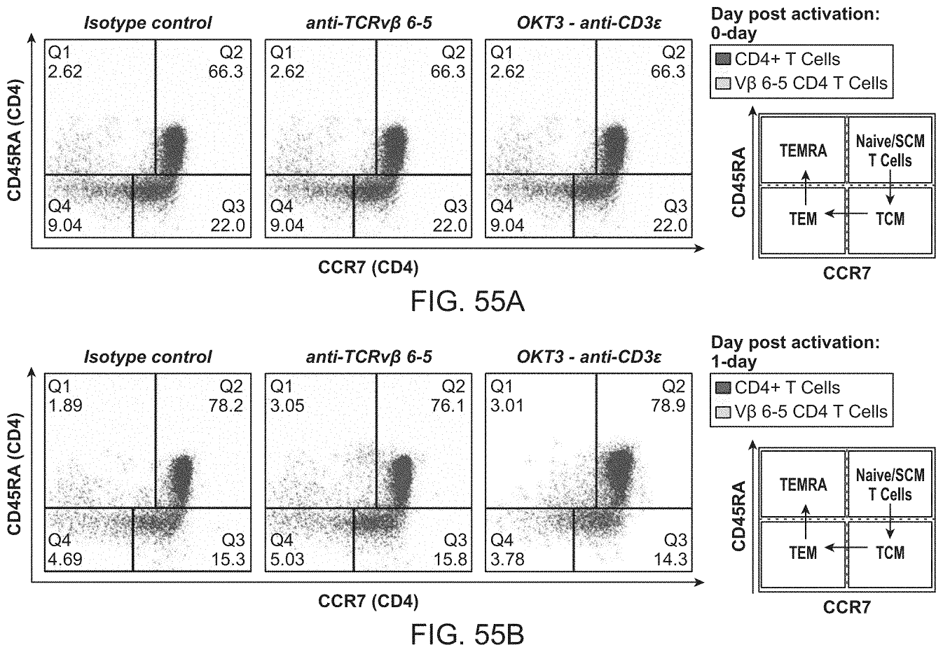

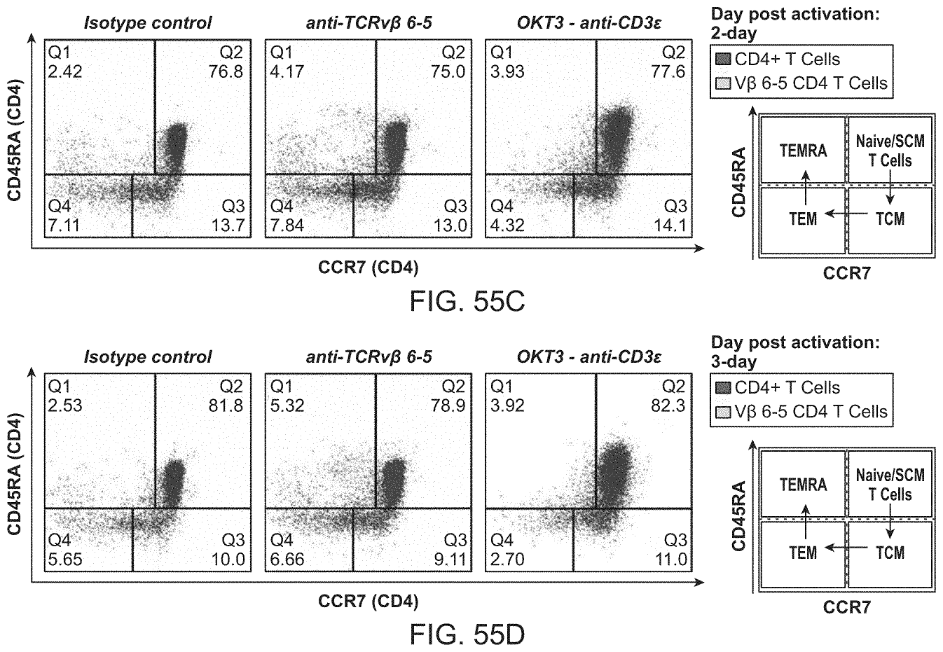

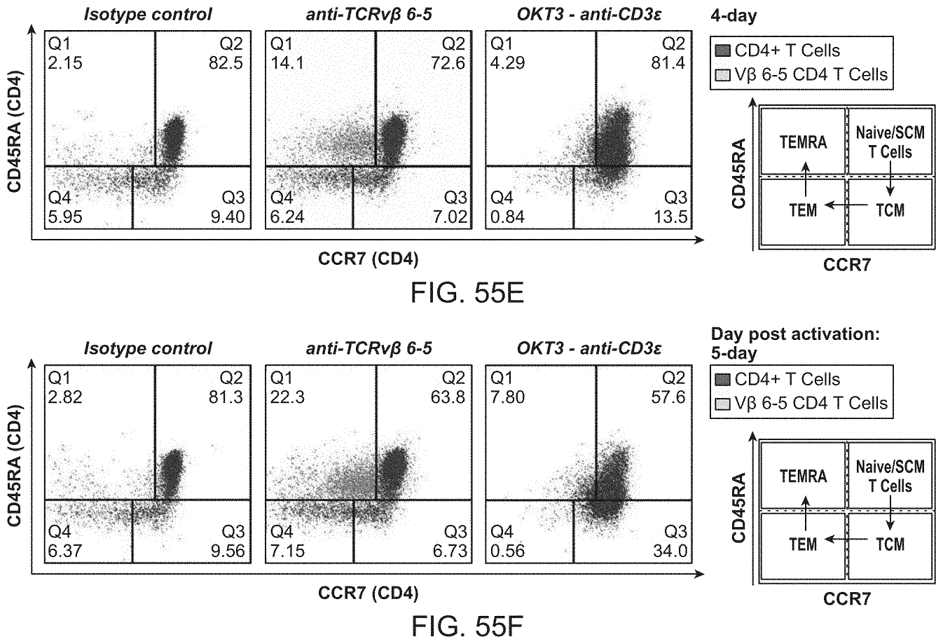

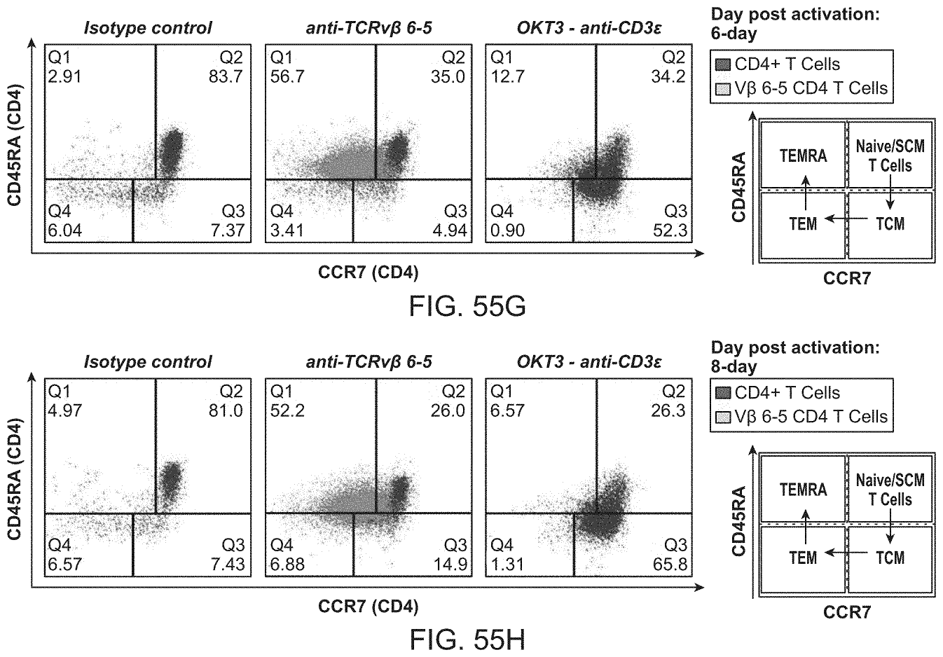

FIG. 54 shows a series of FACS plots showing the percentage of CD3+ (CD4 gated) TCRβV 6-5+ T cells 1, 2, 3, 4, 5, 6, and 8 days port activation with BCMA and the anti-TCR Vβ antibody anti-TCR Vβ 6-5 v1. Analysis of the percentage of CD4+ T cells expanded using isotype control (IgG1 N297A), anti-TCRβV (anti-TCR Vβ 6-5 v1), or anti-CD3ε (OKT3) antibodies on day 0 post activation (FIG. 55A), day 1 post activation (FIG. 55B), day 2 post activation (FIG. 55C), day 3 post activation (FIG. 55D), day 4 post activation (FIG. 55E), day 5 post activation (FIG. 55F), day 6 post activation (FIG. 55G), and day 8 post activation (FIG. 551I). The percentage of TEMRA like T cells expressing both CD45RA and CCR7 shows an increase in the population of TEMRA like cells in the CD4+ TCR Vβ 6-5+ T cell cultures expanded with the anti-TCR Vβ 6-5 v1 antibody compared to those expanded with the OKT3 antibody. Similar results were seen with CD8+ T cells. The results further show that purified human T-cells activated by anti-TCRβV 6-5 directly differentiates to TEMRA subsets and proliferate when compared to purified T-cells activated by anti-CD3ε (OKT3).

In summary, the data shows anti-TCRβV antibodies activated and expanded T cells represent a novel subset of recently activated effector T cells which share phenotypic markers with T_{EMRA}. This is in contrast to anti-CD3e-expanded T cells which differentiated into T_{CM} and T_{EM}. TCRβV expanded T cells are highly proliferative and do not upregulate the senescent marker CD57 OX40, 4-1BB, and ICOS are upregulated on anti-TCRβV activated T cells.

### Example 11: Expression Level of TCRβV6-5 on Jurkat Cells Through Multiple Passages

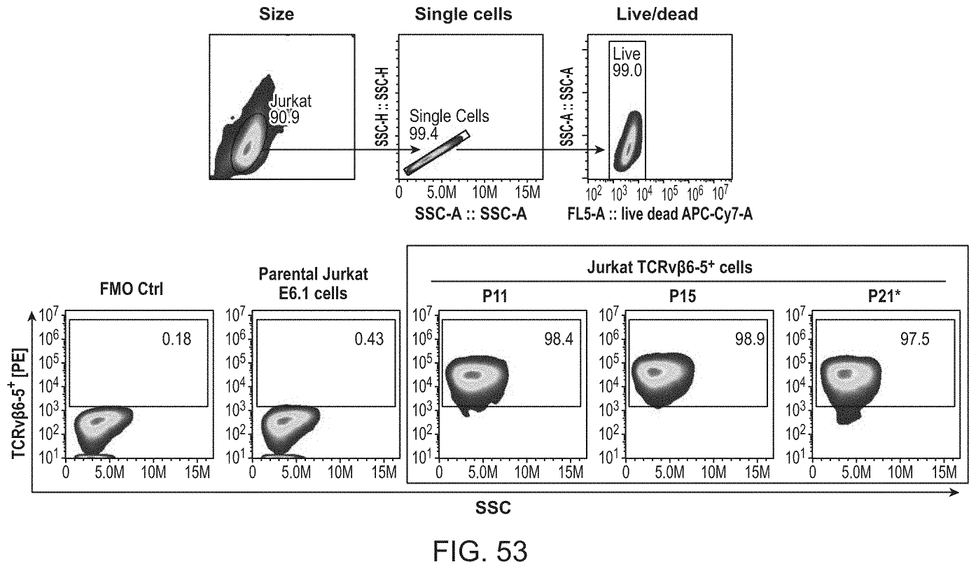

To assess the effect of passage number and culture conditions of TCRβV6-5+ Jurkat on the expression level of TCRβV6-5 TCRβV+ Jurkat cells were maintained in IMDM growth media containing 10% Hi-FBS, 2 mM L-Glu, 1% Pen/Strep, 55 μM BME and parental E6.1 Jurkat cells in RPMI growth media containing 10% Hi-FBS at cell densities between 1×105 and 1×106 cells/mL. The cells were harvested and counted using AOPi staining solution (Nexcelom). 2×10⁵ cells (96-97% viability) were plated per well of a 96-well V-bottom plate and washed twice with PBS. Cells were incubated in 100 μL viability dye (eBioscience Fixable Viability Dye eFluor 780, Cat #65-0865-14, 1:1000 dilution in PBS) for 30 minutes at 4 C in the dark. Cells were washed twice in PBS and incubated in 100 μL of the commercial anti-TCRβV 6-5-PE Ab (Beckman Coulter, Cat #IM2292, 1:10 dilution in FACS buffer (PBS +0.5% BSA)) for 30 minutes at 4 C in the dark. For compensation, beads were stained with respective viability dye or Ab and incubated for 30 minutes at 4 C in the dark. Cells and beads were washed twice in FACS buffer and incubated in 100 μL fixation buffer (4% PFA in PBS, Biolegend, Cat #420801) and incubated for 30 minutes at 4 C in the dark. Cells were washed twice in FACS buffer and the cells and beads were resuspended in 120 μL FACS buffer and acquired on the Cytoflex S for analysis. The data shows that Passage number and culture conditions of TCRβV6-5⁺ Jurkat cells do not affect the expression levels of TCRβV6-5 (FIG. 53).

### Example 12: Differential Gene Expression in Anti-TCRβV Activated Cells

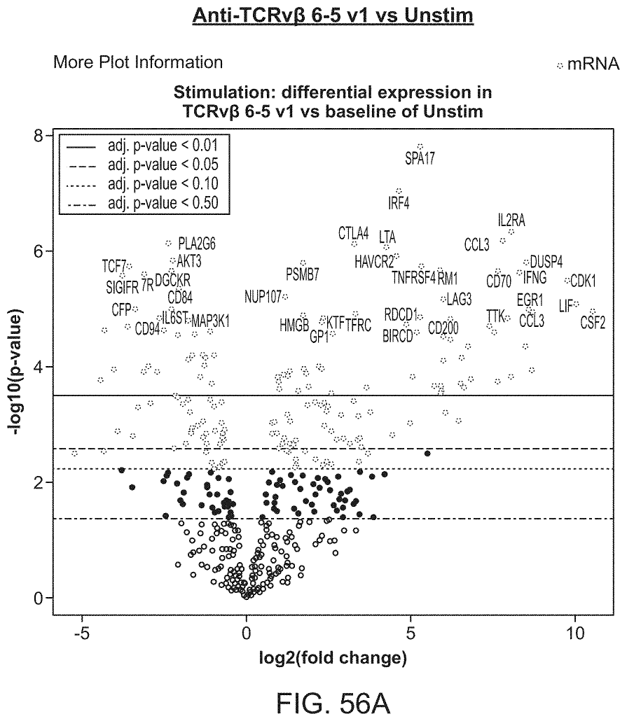

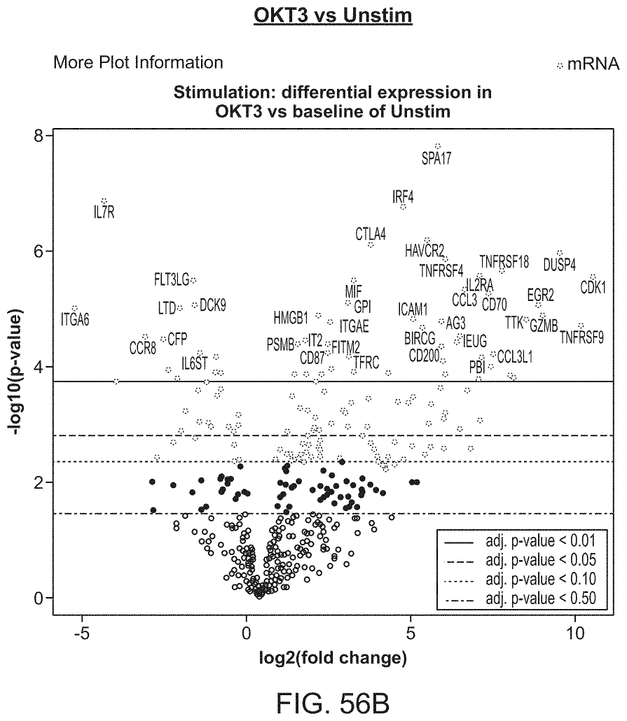

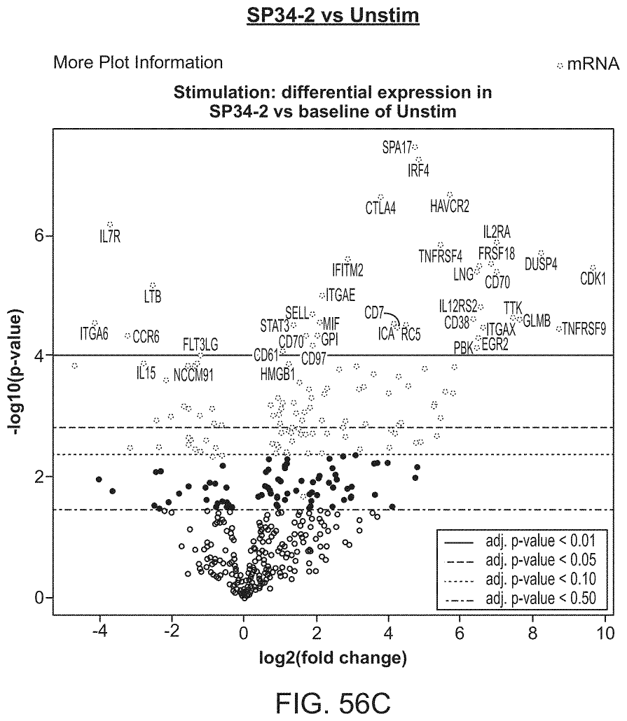

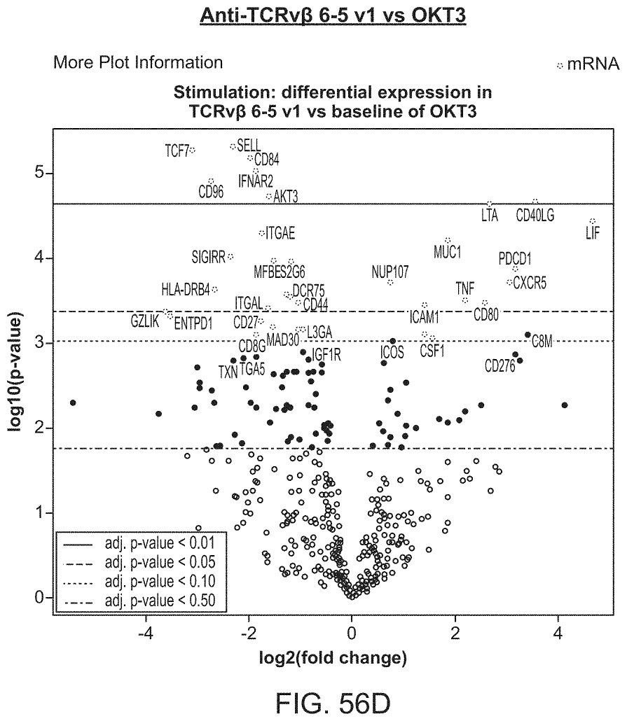

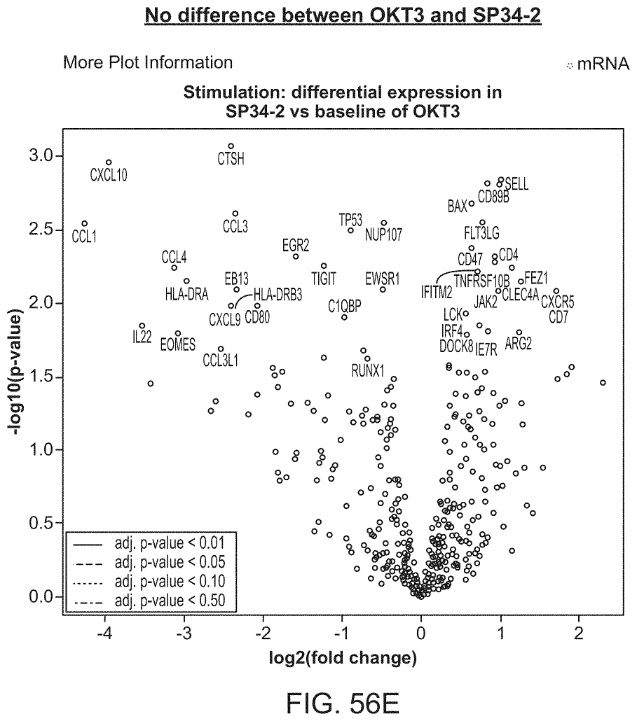

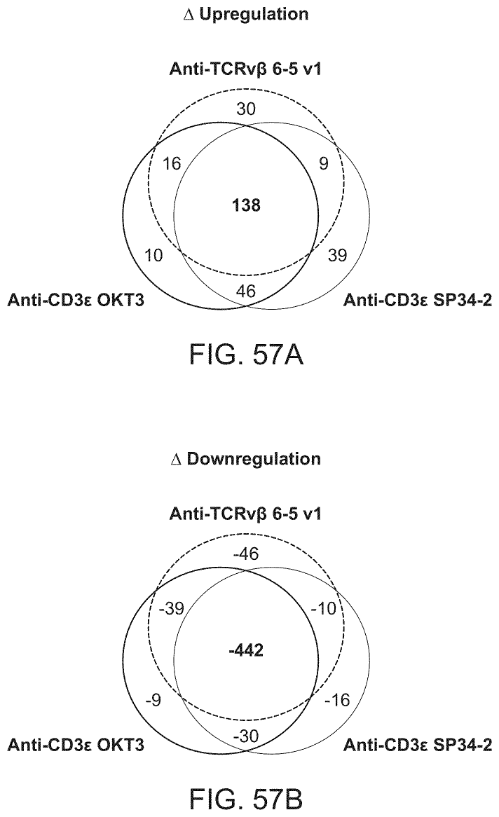

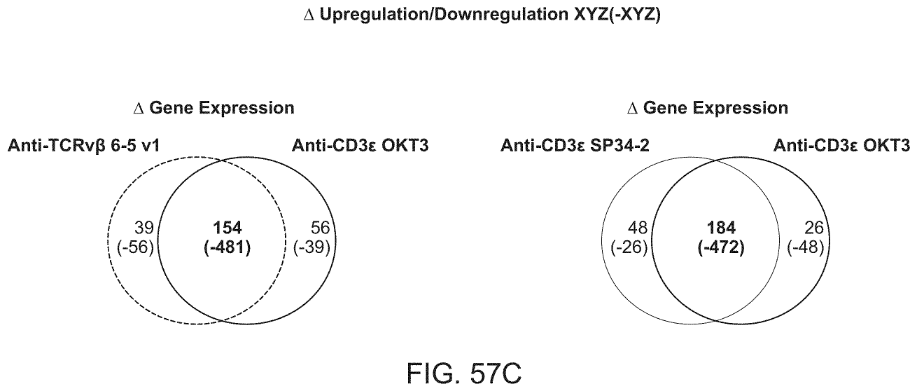

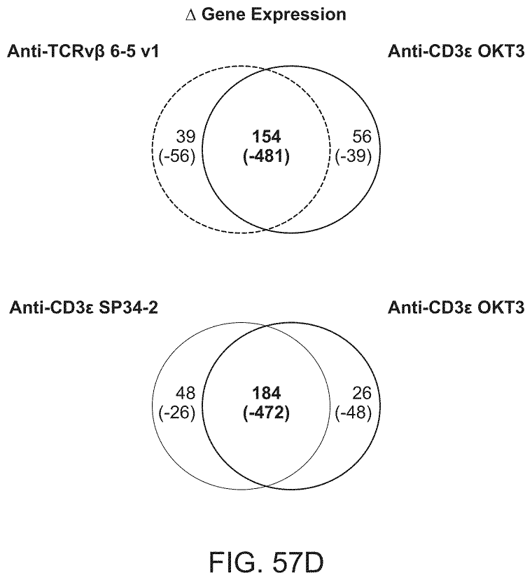

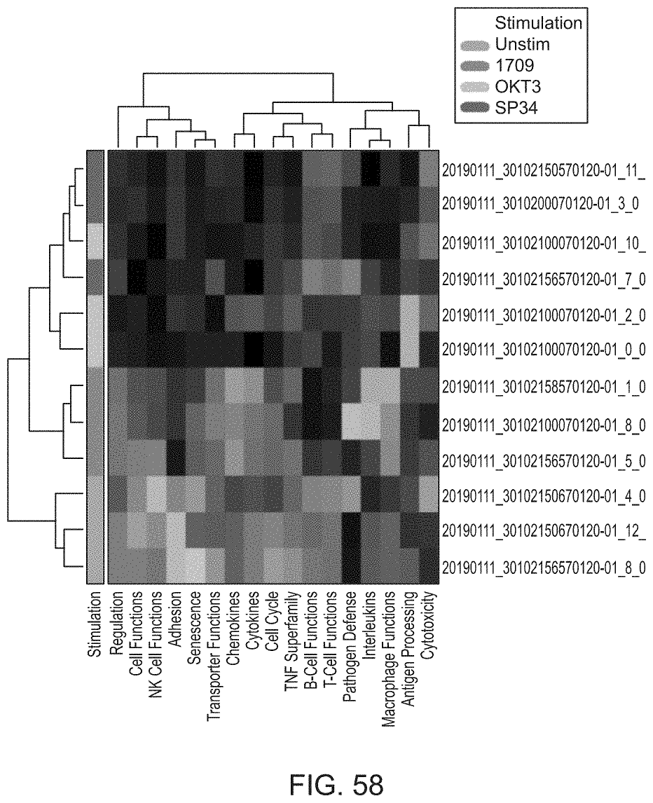

Purified T cells were stimulated with solid-phase anti-TCRβV antibody over 6 days with the indicated T cell-activating antibody at 100 nM: i) anti-TCRvb 6-5 v1 antibody; ii) OKT3 (anti-CD3ε antibody); or iii) SP34-2 (anti-CD3ε antibody). Expanded T cells were collected by centrifugation followed by RNA extraction. 778 immunology-related genes were counted using the nCounter Technology (Nanostring) followed by gene expression analysis using nSolver analysis tools. Data is representative of 3 donors. Genes were found to be differentially expressed between cells activated with anti-TCRvb 6-5 v1 antibody versus unstimulated (FIG. 56A); cells activated with OKT3 versus unstimulated (FIG. 56B); cells activated with SP34-2 versus unstimulated (FIG. 56C); and cells activated with anti-TCRvb 6-5 v1 antibody versus OKT3 (FIG. 56D). While, no differential gene expression was detected between cells activated with OKT3 versus SP34-2 (FIG. 56E). The majority of genes differentially expressed were found to be similar among different activated T cells (FIG. 57A 57D). FIG. 58 shows a heat map of pathway scores for genes differentially regulated and related to various cellular pathways. The purified T cell samples include unstimulated (n=3), OKT3 stimulated (n=3), SP34-2 stimulated (n=3), and anti-TCRβV 6-5 v1 stimulated (n=3). Similar patterns between OKT3 simulated and SP34-2 stimulated T-cells was observed.

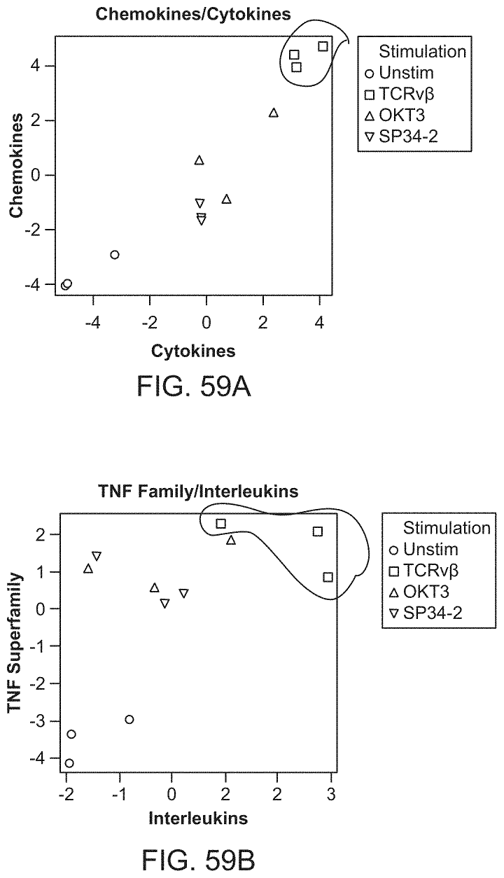

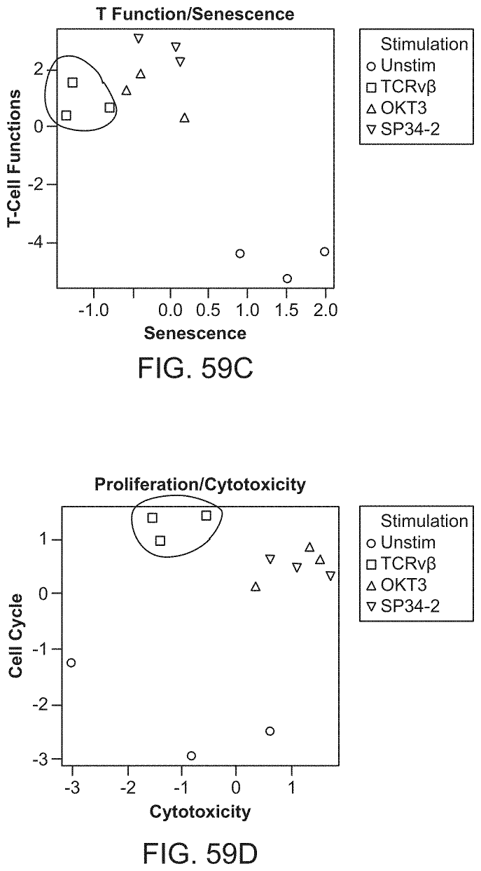

FIG. 59A 59D show the pathways upregulated or downregulated by activation with the indicated antibodies or unstimulated, including cytokines and chemokine pathways (FIG. 59A); TNF superfamily and interleukin pathways (FIG. 59B); T cell function and senescence pathways (FIG. 59C); and cell cycle and cytotoxicity pathways (FIG. 59D).

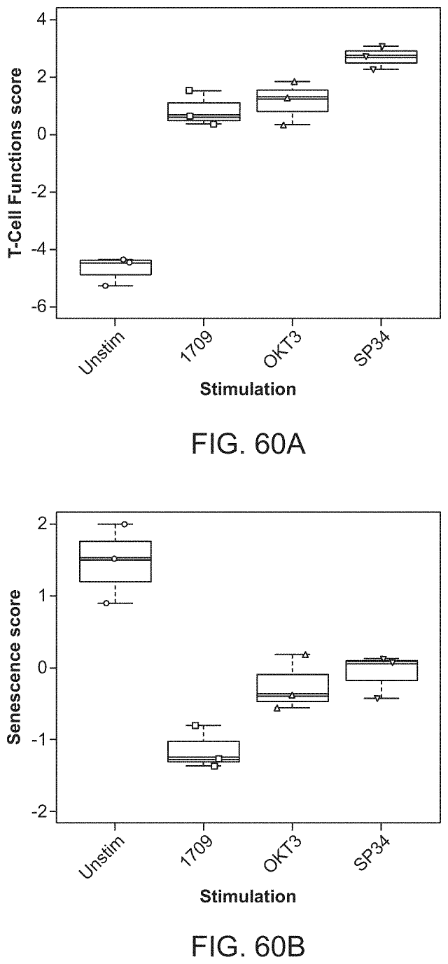

FIG. 60A show the overall pathway score of genes in the T cell function pathway differentially expressed by activation with the indicated antibodies; and FIG. 60B show the overall pathway score of genes in the senescence pathway differentially expressed by activation with the indicated antibodies. The data shows that αTCRβV 6-5 v1 activated T cells are functional and viable.

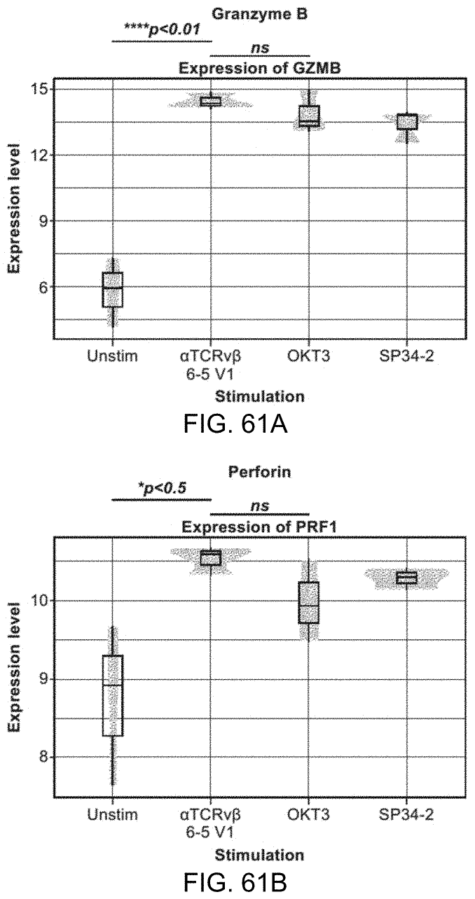

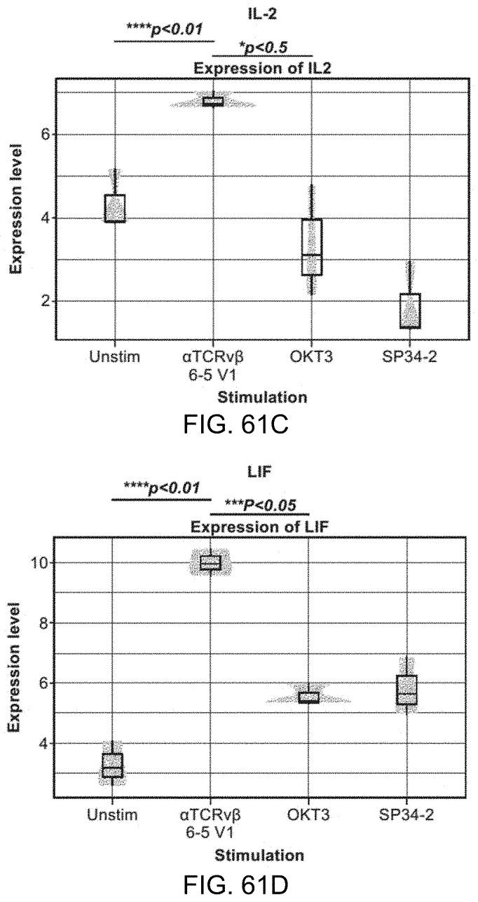

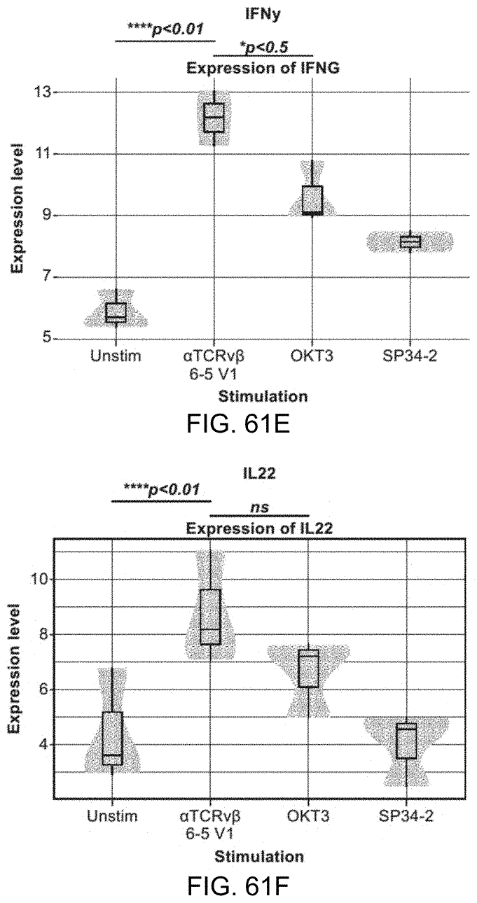

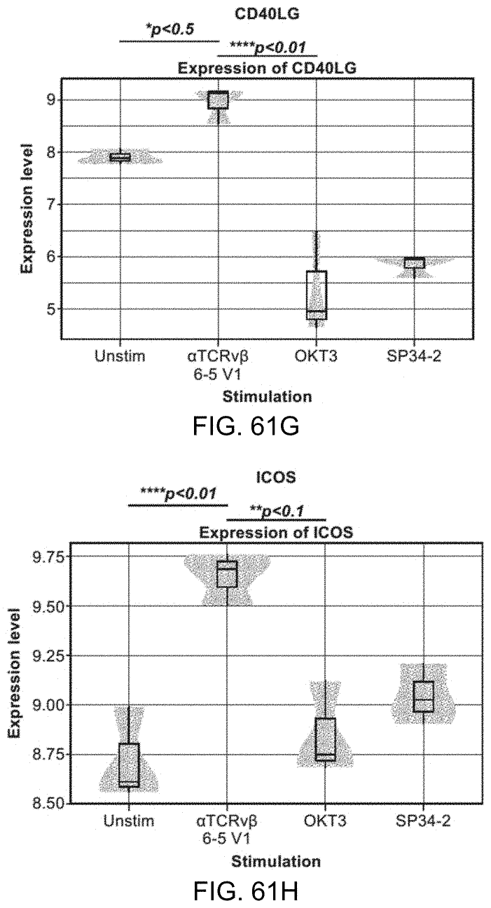

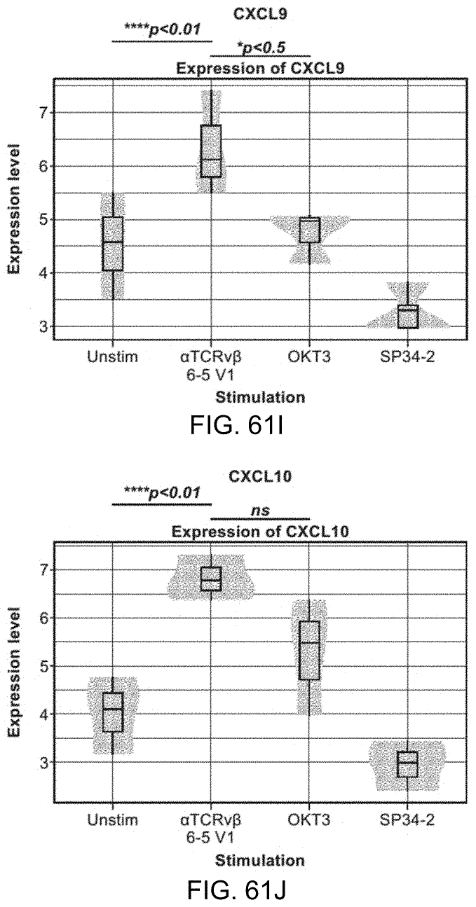

FIG. 61A-FIG. 61J show the differential regulation of genes in cells activated with the indicated antibody, OKT3, SP34-2, or anti-TCRβV 6-5 v1, or unstimulated. The genes analyzed included granzyme B (FIG. 61A) and perforin (FIG. 61B), showing the upregulation of genes associated with cytotoxicity function in cells activated with αTCRβV 6-5 v1 antibody. Increased expression of IL-2 (FIG. 61C) and LIF (FIG. 61D) by T cells activated with anti-TCRβV 6-5 v1 antibody shows the anti-TCRβV 6-5 v1 expanded T cells are highly proliferative. Increased expression of IFNγ (FIG. 61E) and IL-22 (FIG. 61F) by T cells activated with anti-TCRβV 6-5 v1 antibody shows the anti-TCRβV 6-5 v1 expanded T cells are highly active. T cells activated with anti-TCRβV 6-5 v1 antibody shows the anti-TCRβV 6-5 v1 also show increased expression of the co-stimulatory molecules CD40LG (FIG. 61G) and ICOS (FIG. 61H). T cells activated with anti-TCRβV 6-5 v1 antibody shows the anti-TCRβV 6-5 v1 also show increased expression of the IFNγ-mediated antitumor cytokines CCXL9 (FIG. 61I) and CXCL10 (FIG. 61J).

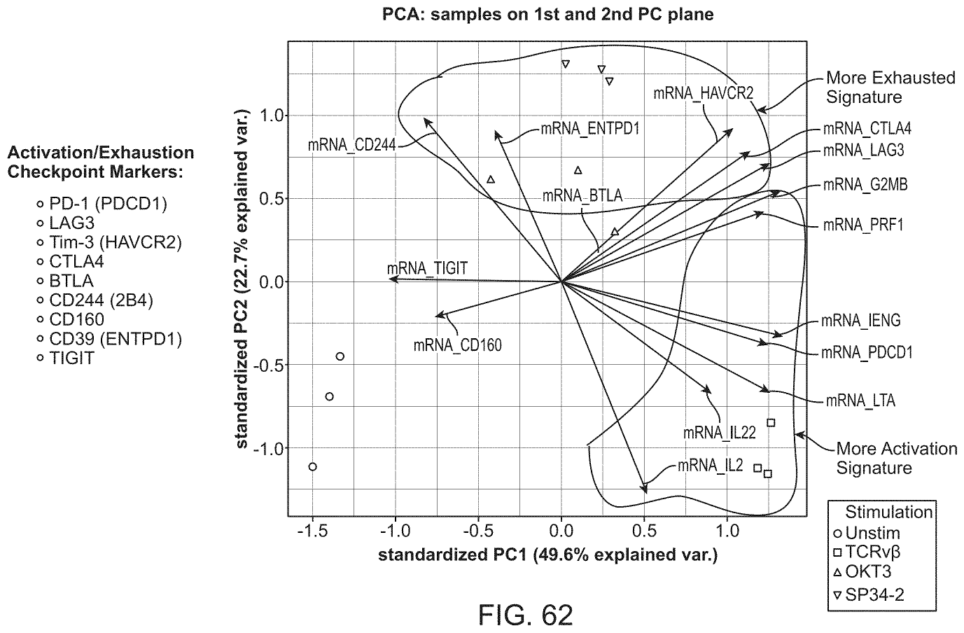

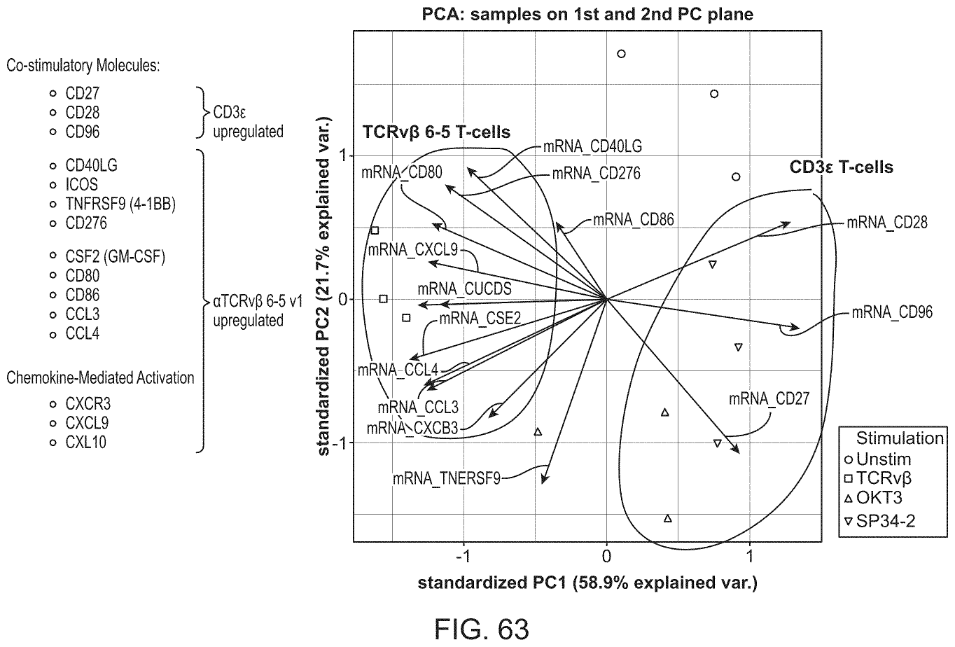

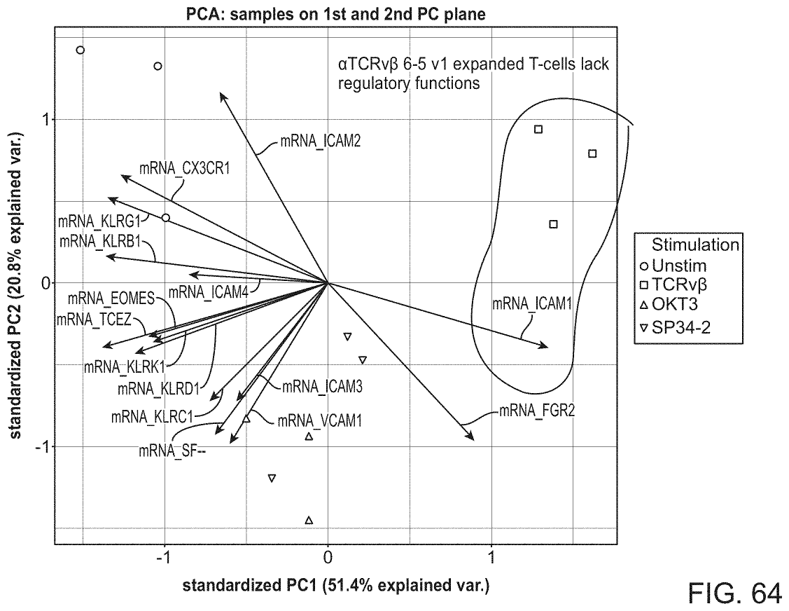

Principal component analysis of activation and exhaustion checkpoint markers PD-1 (PDCD1), LAG3, Tim-3 (HAVCR2), CTLA4, BTLA, CD244 (2B4), CD160, CD39 (ENTPD1), and TIGIT, shows αTCRβV 6-5 v1 expanded T cells appear less exhausted compared to T cells activated with anti-CD3ε antibodies (FIG. 62). Principal component analysis of costimulatory markers CD27, CD28, CD96, CD40LG, ICOS, TNFRSF9 (4-1BB), CD276, CSF2 (GM-CSF), CD80, CD86, CCL3, and CCL4, show differentiation upregulation with CSF2 (GM-CSF), CD80, CD86, CCL3, and CCL4 upregulated in T cells activated with αTCRβV 6-5 v1 antibody; and CD27, CD28, CD96, CD40LG, ICOS, TNFRSF9 (4-1BB), and CD276 upregulated in T cells activated with anti-CD3ε (FIG. 63). The analysis further showed upregulation of chemokine-mediated activation genes CXCR3, CXCL9, and CXCL10 in T cells activated with αTCRβV 6-5 v1 antibody (FIG. 63). Principal component analysis of regulatory genes indicated αTCRβV 6-5 v1 expanded T-cells lack regulatory functions (FIG. 64).

In summary, the data indicate CD3ε- or αTCRβV 6-5 v1-expanded T cells share many differentially expressed genes; and αTCRβV 6-5 v1-activated T cells express high levels of cytolytic effectors, proliferative markers and appear to be less exhausted compared to CD3ε-activated T cells.

### Example 13: Metabolic State of αTCRβV Activated T Cells

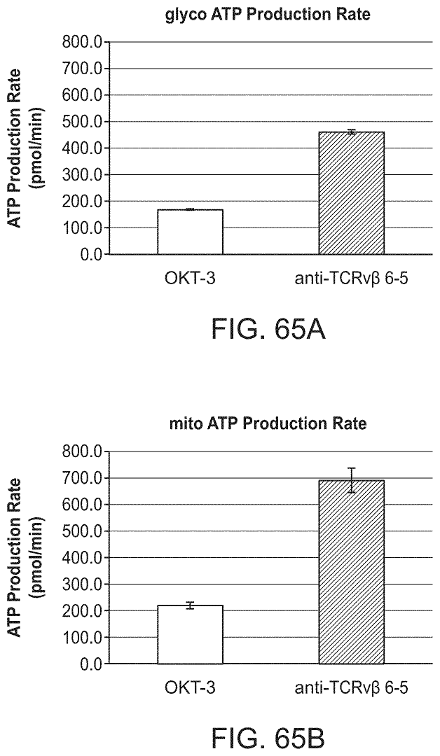

To evaluate the metabolic phenotype of T cells activated with αTCRβV antibodies nave T cells from PBMCs were stimulated and expanded for 5 days with plate-bound anti-CD3 antibody (OKT3) or anti-TCRβV antibody (anti-TCRβV 6-5 v1 antibody). Activated T cells were then rested in IL-2 containing media for 2 days, before they were cryopreserved. Prior to assay setup, cells were thawed and re-stimulated for 3 days with plate-bound anti-CD3 Ab (clone OKT3) or anti-TCRβV antibody (anti-TCRβV 6-5 v1 antibody), respectively. Equal numbers of live cells were plated on a Seahorse cartridge, and the Real-Time ATP Rate Assay was performed according to manufacturer's instructions. The data showed that ATP production from glycolysis (FIG. 65A) oxidative phosphorylation (FIG. 65B) in T cells from 3 donors (representative results from a single donor presented in FIG. 65A-65B) activated with the anti-TCRβV 6-5 v1 antibody increased compared to T cells activated with the OKT3 antibody (3-fold increase in ATP production was observed on average); and one donor showed equal levels of ATP production in anti-TCRβV 6-5 v1 and OKT3 Ab stimulated cells (data not shown).

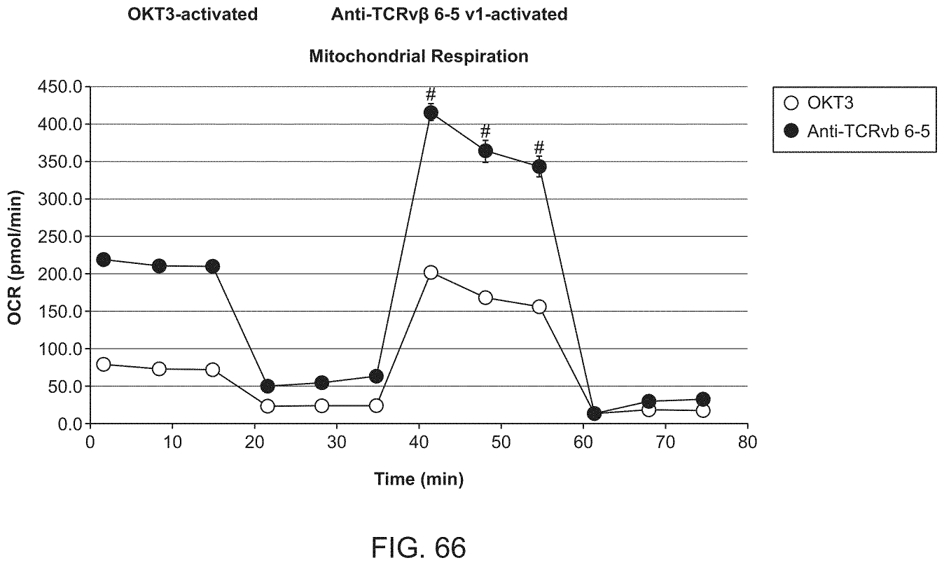

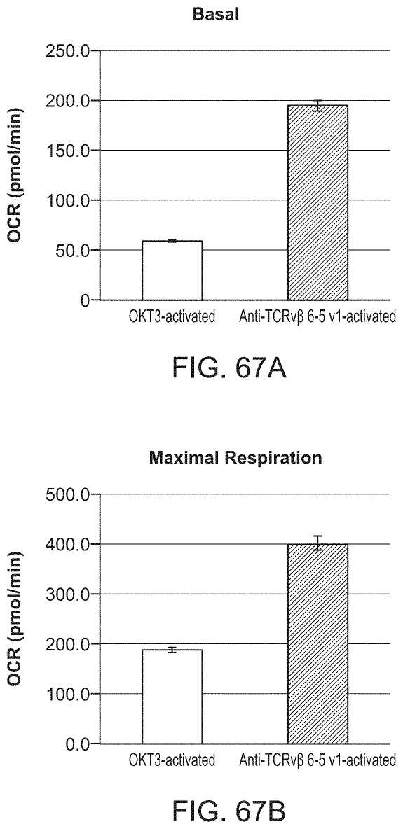

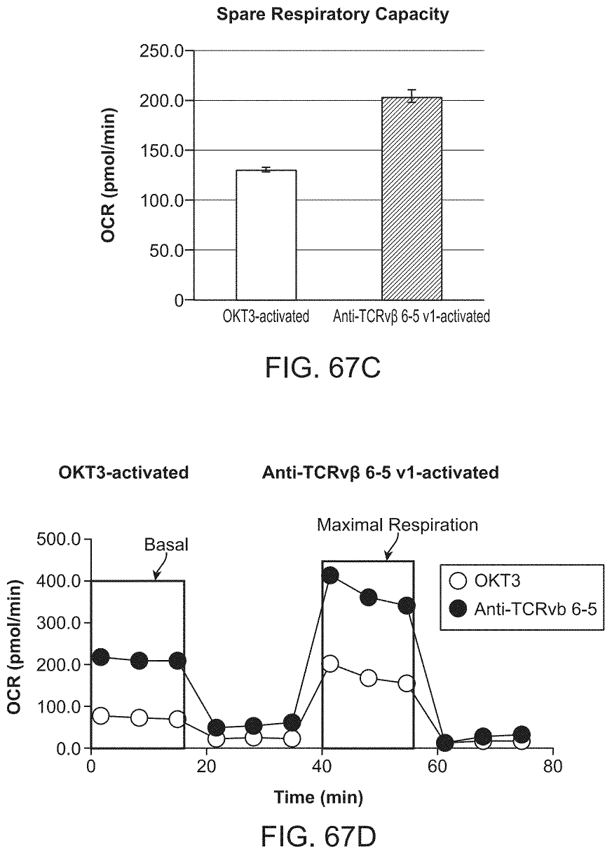

The increased mitochondrial respiration in T cells activated with anti-TCRβV 6-5 v1 antibody compared to T cells activated with the OKT3 antibody is further shown in FIG. 66, which shows the oxygen consumption rate (OCR) of T cells from about 0 to 75 minutes activated with the indicated antibody. Data in FIG. 66 is from a single donor; a second donor tested showed equal levels of ATP production in anti-TCRβV 6-5 v1 and OKT3 Ab stimulated cells (data not shown). FIGS. 67A-67C shows the oxygen consumption rate (OCR) of T cells activated with the indicated antibody during basal respiration (FIG. 67A), maximal respiration (FIG. 67B), and spare respiratory capacity (FIG. 67C). Cells were plated in media containing glucose and glutamine to measure basal OCR. FCCP (ETC accelerator) was added to the cell culture medium to determine maximum respiratory capacity/max OCR. Antimycin A & Rotenone (ETC inhibitor) were added to the cell culture medium to determine spare respiratory capacity and non-mitochondrial oxygen consumption. The data presented in FIGS. 67A-67C α-TCRβV 6-5 v1 activated T cells had significantly increased basal respiration, maximal respiration, and spare respiratory capacity compared to α-CD3 (OKT3) activated T cells (data from a single donor). A second donor was tested which showed equal levels of ATP production in anti-TCRβV 6-5 v1 and OKT3 Ab stimulated cells (data not shown).FIG. 67D indicates the areas of basal respiration and maximal respiration as shown in FIG. 67A and FIG. 67B, respectively.

In order to determine if the observed increase in metabolism due to differences in T cell stimulation, or is it intrinsic to the differentiation stage of T cells activated with anti-TCRβV antibodies TCRβV 6-5+ T cells were expanded for 5 days with plate-bound anti-TCRβV 6-5 v1 Ab. Cells were then rested in IL-2 containing media for 2 days and cryopreserved. Upon thawing, cells were re-stimulated with anti-TCRβV 6-5 v1 for 3 days. Cells were then counted and equal numbers of live cells were re-seeded and stimulated with plate-bound anti-CD3 Ab (clone OKT3) or anti-TCRβV 6-5 v1, respectively, for 24 hours. Equal numbers of live cells were plated on the Seahorse cartridge and the Real-Time ATP Rate Assay was performed.

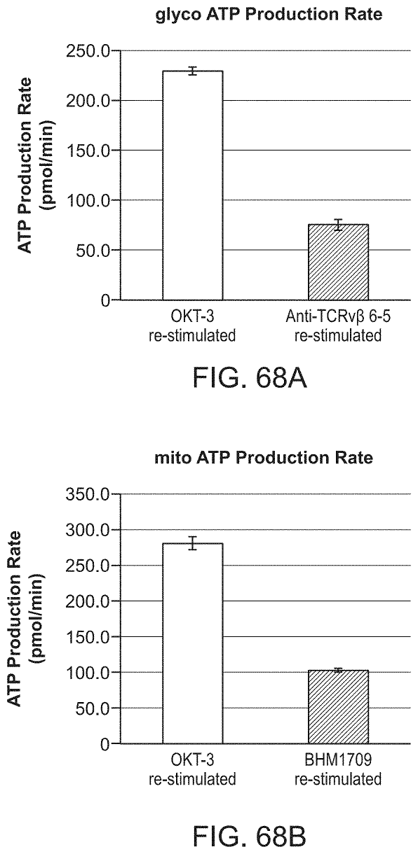

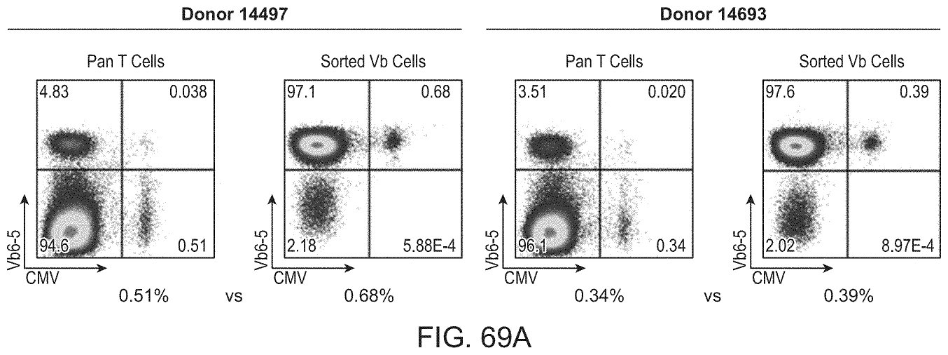

The results show that ATP production by glycolysis (FIG. 68A) and oxidative phosphorylation (FIG. 68B) by T cells activated with anti-TCRβV 6-5 v1 is significantly increased upon re-stimulation with α-CD3 antibody OKT3 versus α-TCRβV 6-5 v1 antibody. The observed increase in metabolism of T cells activated with anti-TCRβV 6-5 v1 appears to be due to intrinsic differences upon differentiation into these cells. T cells activated with anti-TCRβV 6-5 v1 have an increased metabolism compared to CD3-activated T cells, which can be further enhanced with strong T cell stimulation via OKT3.

In summary, the results show that T cells activated with anti-TCRβV antibodies have a metabolic memory phenotype. The cells are not metabolically exhausted, because exhausted T cells have a decreased metabolism. α-TCRβV 6-5 v1-stimulation induces a T cell differentiation stage, which is highly metabolically active, indicative of an effector memory phenotype. This metabolic phenotype is maintained when these cells are re-stimulated with other T cell engagers (OKT3).

### Example 14: TCRβV 6-5+ T Cells do not Represent Virus-Specific Memory T Cells

To assess whether TCRβV 6-5+ T cells represent virus-specific memory T cells—TCRβV 6-5+ T cells were prepared using two different methods. Method 1: total CD3 T-cells were first isolated via magnetic bead negative selection (Miltenyi Biotec), followed by FACS sorted TCRβV 6-5+ T cells (with >95% purity) or pan T-cells were activated with microbeads (at 2:1 T-cell:bead ratio) coated with anti-CD2/CD3/CD28 antibodies (Miltenyi Biotec, 10 ug per antibody per 100 million beads) and recombinant human IL-2 (Roche, 20 U per ml) for 6 days; and activated/expanded TCRβV 6-5+ T cells were stained for viral specific tetramer's that are HLA-matched to donor cells, and analyzed by flow cytometry. Method 2: total CD3 T-cells were first isolated via magnetic bead negative selection (Miltenyi Biotec), and then total T-cells were stimulated with plate-bound anti-TCRβV 6-5 antibody v1 (100 nM) or OKT3 (100 nM) for 6 days, followed by the addition of rhIL-2 (Roche, 50 U per ml) for 2 more days; and activated/expanded TCRβV 6-5+ T cells were stained for viral specific tetramer's that are HLA-matched to donor cells, and analyzed by flow cytometry.

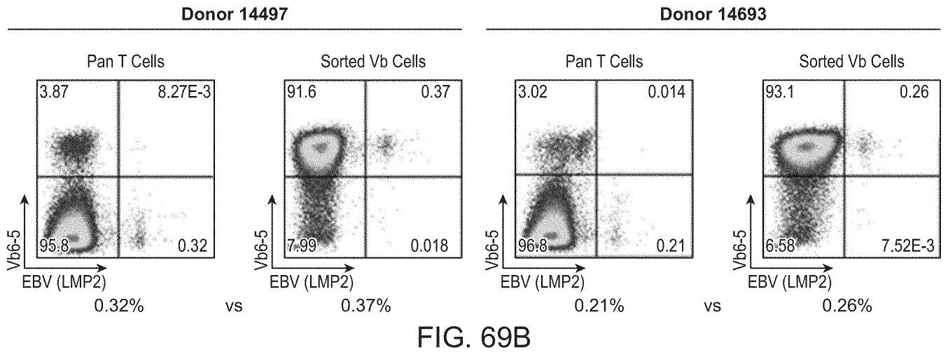

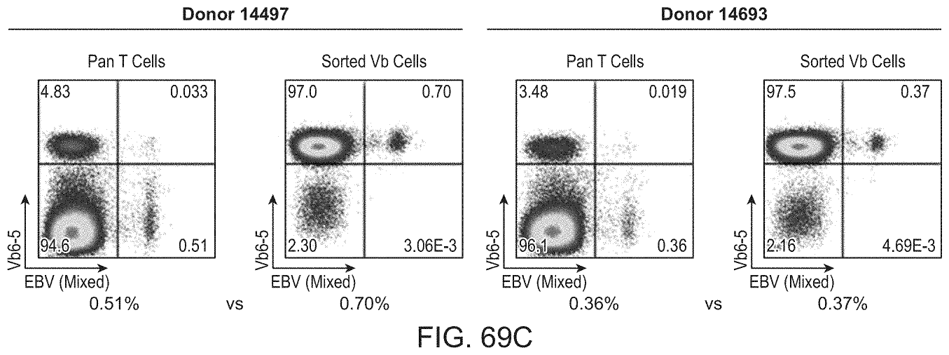

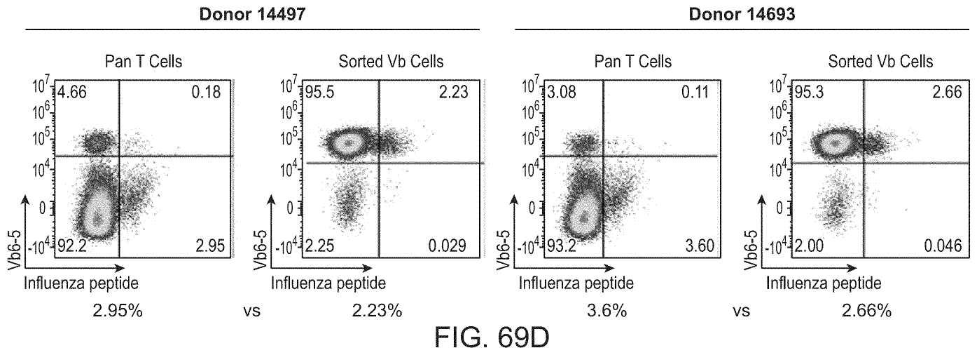

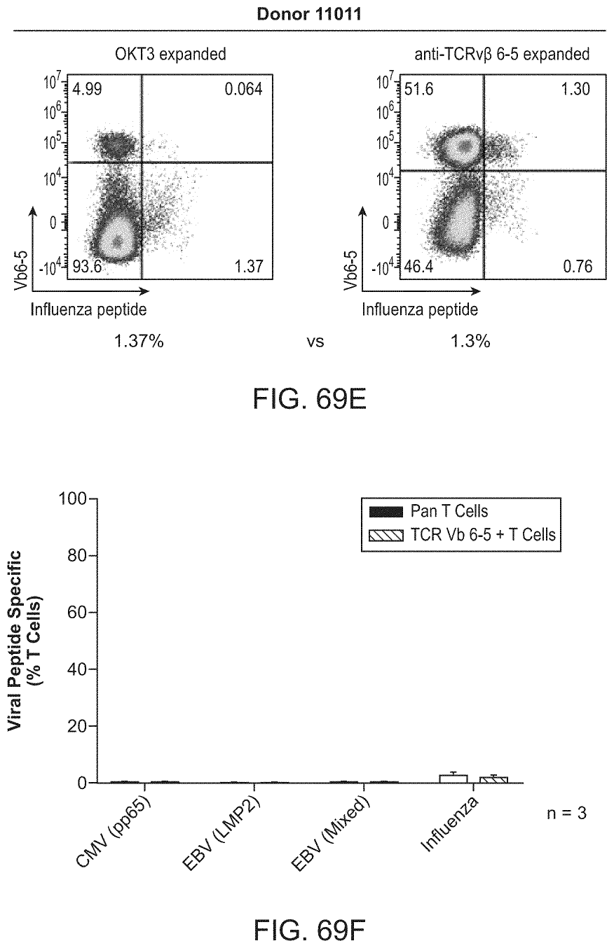

The data show that TCRvβ 6-5+ CD8+ T cells are not CMV (pp65) specific (FIG. 69A) (Method 1); EBV (LMP2) specific (FIG. 69B) (Method 1); EBV (mixed peptide) specific (FIG. 69C) (Method 1); influenza specific (FIG. 69D (Method 1); FIG. 69E (Method 2)). A summary of the results is further provided in FIG. 69F. In summary, the data show that TCRβV 6-5+ T cells do not appear to represent commonly viral specific (CMV, EBV and influenza) specific CD8+ T cells. Both methods described above (Method 1 and Method 2) show similar peptide binding results.

### Example 15: Anti-TCRβV Stimulated PBMC Mediated Stimulation of NK Cell Expansion

To assess whether anti-TCRβV stimulated PBMCs mediate expansion of NK cells in vitro human PBMCs were stimulated with 100 nM of plate-coated anti-TCRβV 6-5 v1 anti-CD3ε (OKT3 and SP34-2) for up to 7 days. NK cells were identified via FACS staining for CD3-/CD56+/CD16+/NKp46+ populations. NK cell count was determined by a constant μl sample (presented as relative count for each donor). NK cell-mediated target cell lysis was determined 6-days post stimulation, in which PBMCs were harvested and co-cultured with K562 target cells for 4 hours to determine cell killing, via DRAQ7 viability FACS staining.

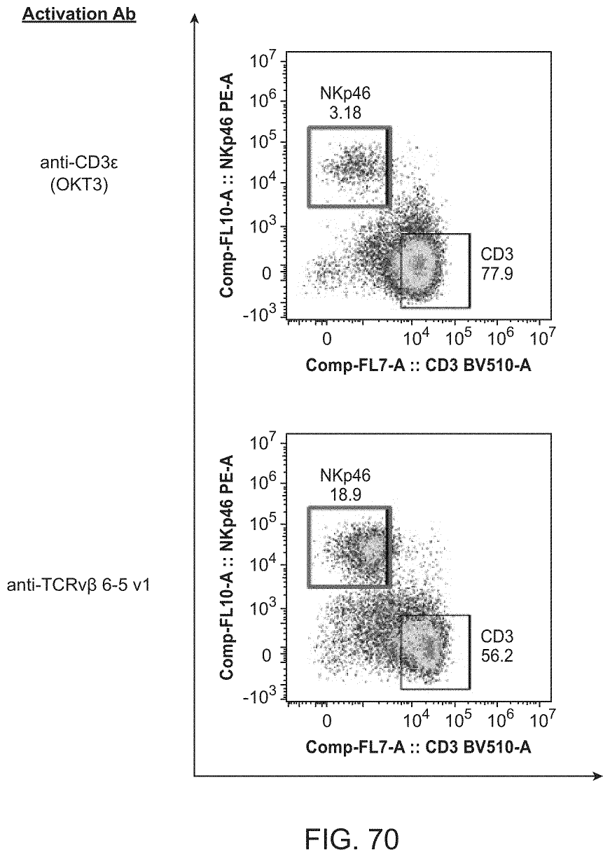

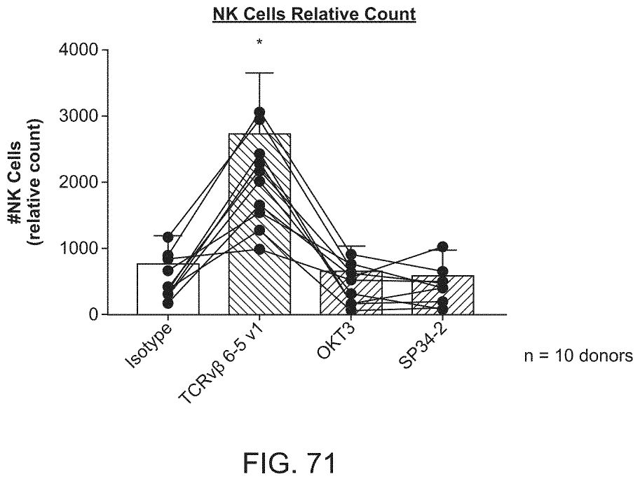

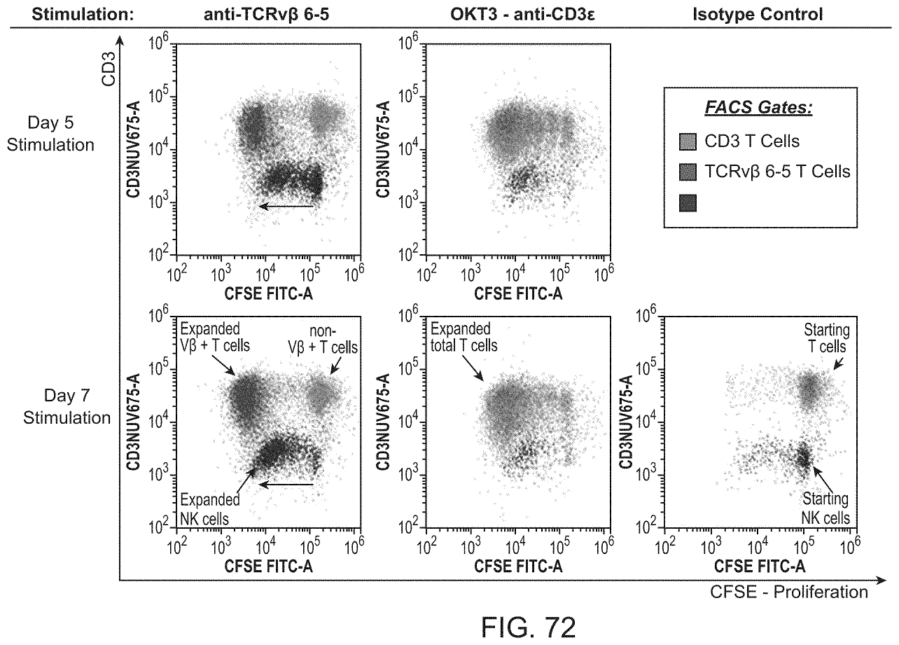

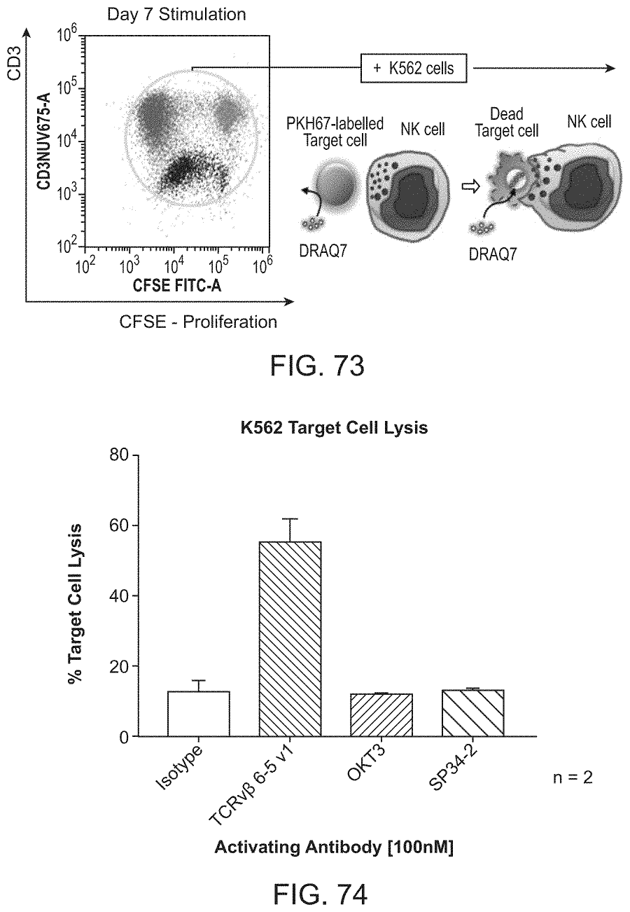

The results show that anti-TCRβV stimulation increases NK cell numbers compared to OKT3 stimulation (FIG. 70; FIG. 71). FACS CFSE staining further shows NK cell proliferation (FIG. 72). FIG. 73 and FIG. 74 shows NK cell mediated lysis of target K562 cells. In summary, anti-TCRβV 6-5 antibody induces expansion of NK cells in PBMC; and This effect is unlikely to be mediated through the FcR on NK cells as anti-CD3ε antibodies did not expand NK cells. Expanded NK cells by anti-TCRβV 6-5 v1 mediates potent target cell (K562) lysis in vitro.

In addition to the experiments conducted above using the anti-TCRβV 6-5 v1 antibody, similar experiments were carried out using anti-TCRβV antibodies that recognize different clonotypes. In one experiment, the anti-TCRβV 12 antibodies: anti-TCRvβ 12-3/4 v1, anti-TCRvβ 12-3/4 v2, and anti-TCRvβ 12-3/4 v3 were used to activate/expand PBMCs using solid-phase stimulated (plate-coated) with the indicated T cell-activating antibody at 100 nM for 6 days as described above. Flow analysis was performed for NK cells using NKp46 and CD56 (CD3 negative). Data was generated from 3 donors and representative of 1 independent experiments.

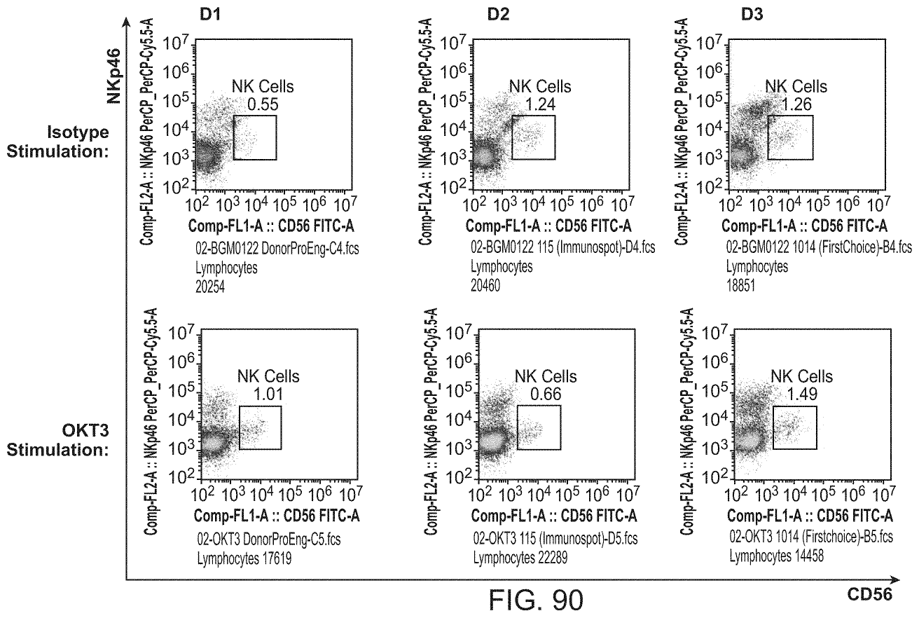

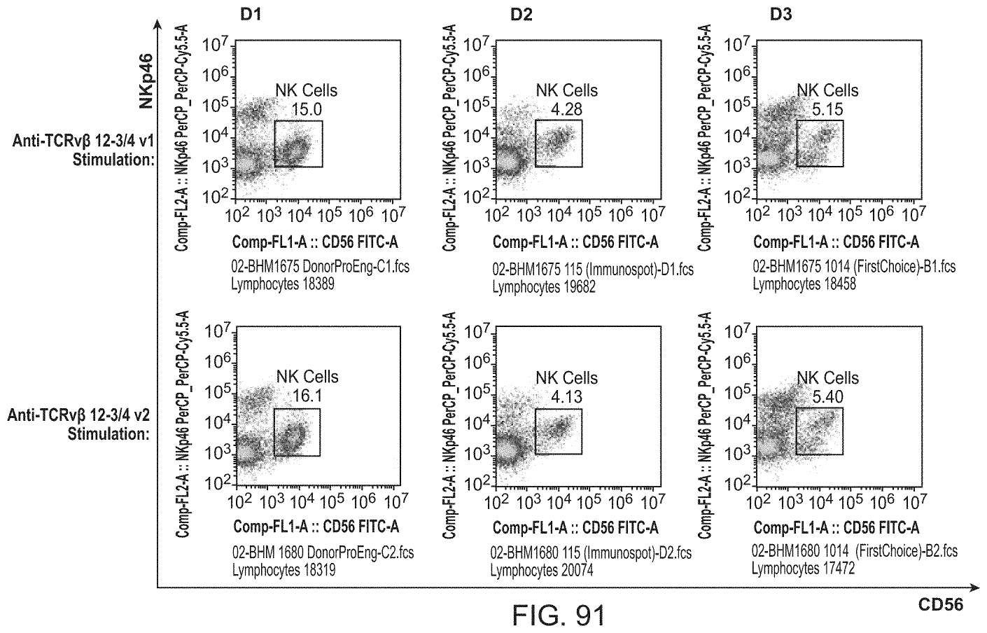

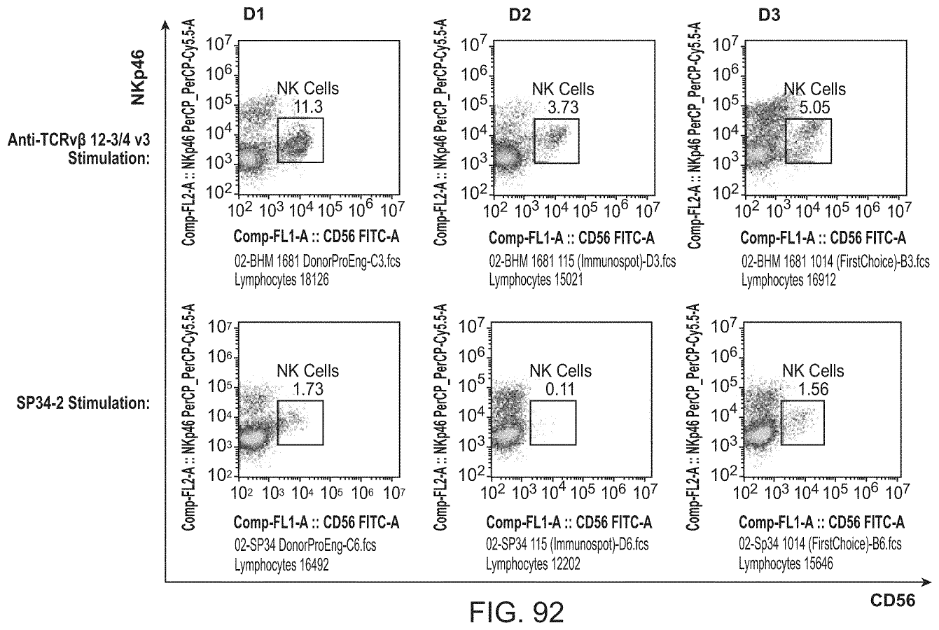

Activation/expansion of the PBMCs with isotype control or the anti-CD3ε antibody OKT3 or SP34-2 did not induce expansion of NK cells (FIG. 90; FIG. 92). However, activation/expansion of PBMCs with anti-TCRvβ 12-3/4 v1 (FIG. 91), anti-TCRvβ 12-3/4 v2 (FIG. 91), and anti-TCRvβ 12-3/4 v3 (FIG. 92) all induced NK cell expansion. In summary, the data shows that anti-TCRvb 12 antibodies are able to induce indirect expansion of NK cells from PBMC cultures in vitro.

### Example 16: Concentration Response to Anti-TCRβV Stimulation In Vitro

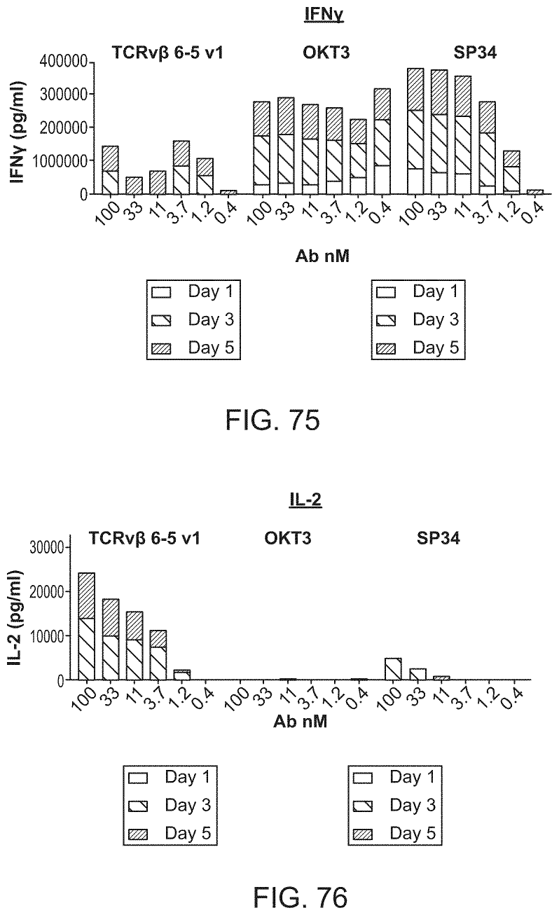

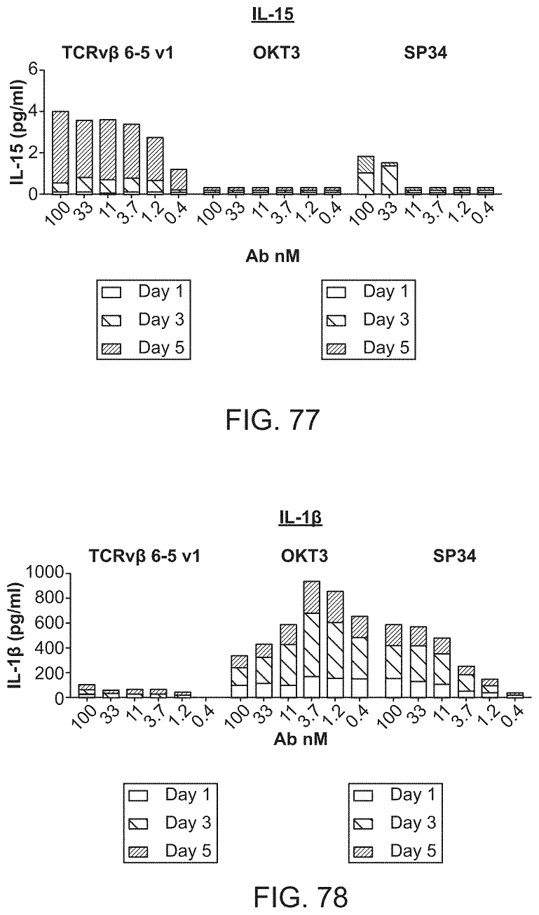

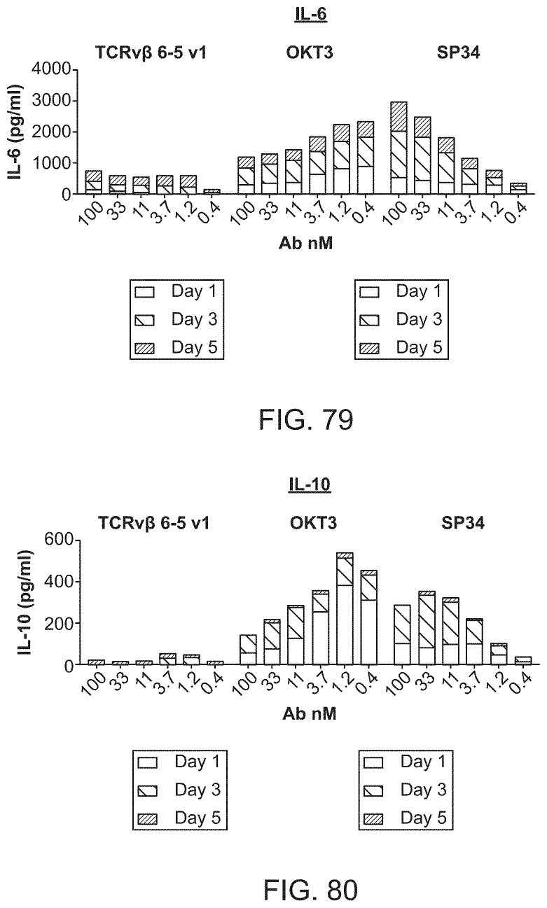

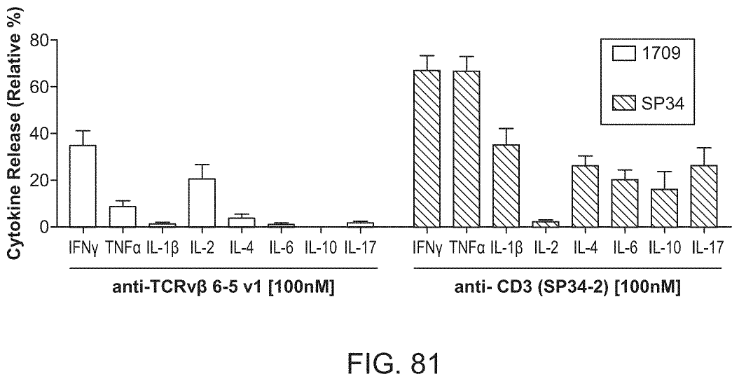

Human PBMCs were solid-phase stimulated (plate-coated) with the indicated T cell-activating antibody at the indicated different concentrations: i) anti-TCRvb 6-5 v1 antibody; ii) OKT3 (anti-CD3ε antibody); or iii) SP34-2 (anti-CD3ε antibody). Supernatant were collected on day 1, day 3 and day 5 and cytokines quantified by using Meso Scale Discovery (MSD) assay. The production of cytokines IFNγ (FIG. 75), IL-2 (FIG. 76), IL-15 (FIG. 77), IL-1β (FIG. 78), IL-6 (FIG. 79), and IL-1β (FIG. 80) was analyzed. The results indicate that the lack of CRS associated cytokine induction by T cells activated with an anti-TCRvb is not a result of inhibition or toxicity due to high antibody concentrations.

### Example 17: T Cells Activated by Anti-TCRβV Antibodies have a Distinct Cytokine Release Profile Compared to T Cell Activated with Anti-CD3ε Antibodies

To assess the cytokine release profile of T cells activated/expanded using anti-TCRβV antibodies as compared to anti-CD3ε antibodies PBMCs were cultured in cell culture plates coated with the immobilized anti-TCRβV antibody anti-TCRβV 6-5 v1 or an anti-CD3ε antibody, either OKT3 or SP37-2. The cells were cultured for 1-8 days, the supernatant collected, and cytokines analyzed using Meso Scale Discovery (MSD) assay. T cells samples from numerous different human donors were tested.

FIG. 81 shows a summary of data from 17 donors. The highest overall cytokine secretion from time points (day 3 and beyond) was used for further analysis. Each data point was normalized against the highest secretion for each donor and showed as relative % of highest (at a confidence interval of 0.95 percentile). The data shows that T cells activated/expanded with an anti-TCRβV antibody as compared to anti-CD3ε antibody release less IFNγ, TNFα, IL-1β, IL-4, IL-6, IL10, and IL-17; while releasing an increased amount of IL-2 (FIG. 81).

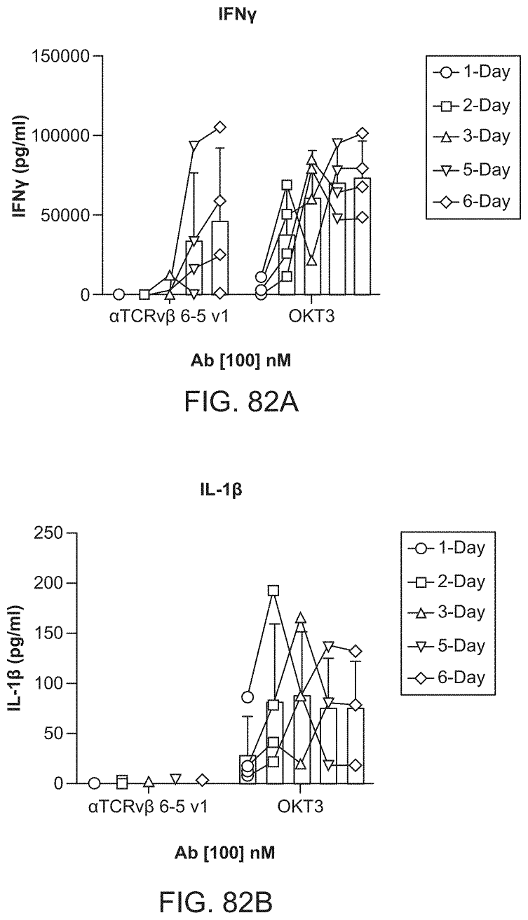

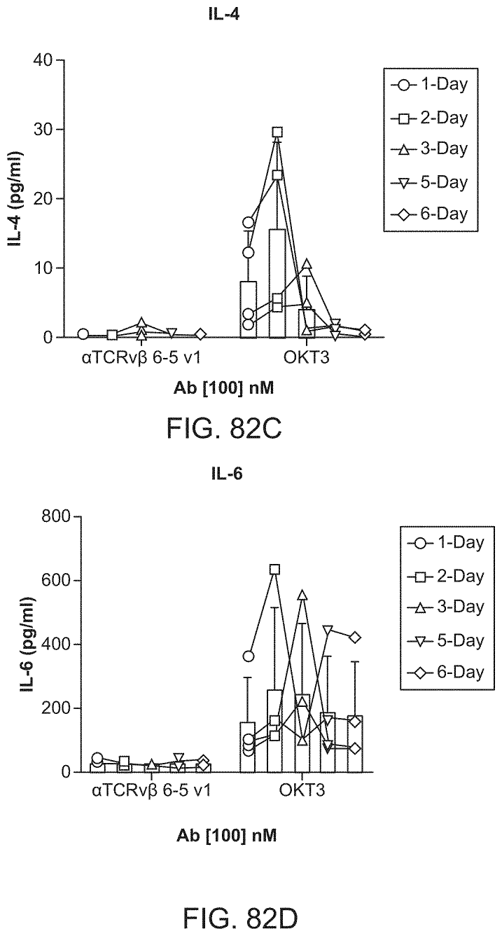

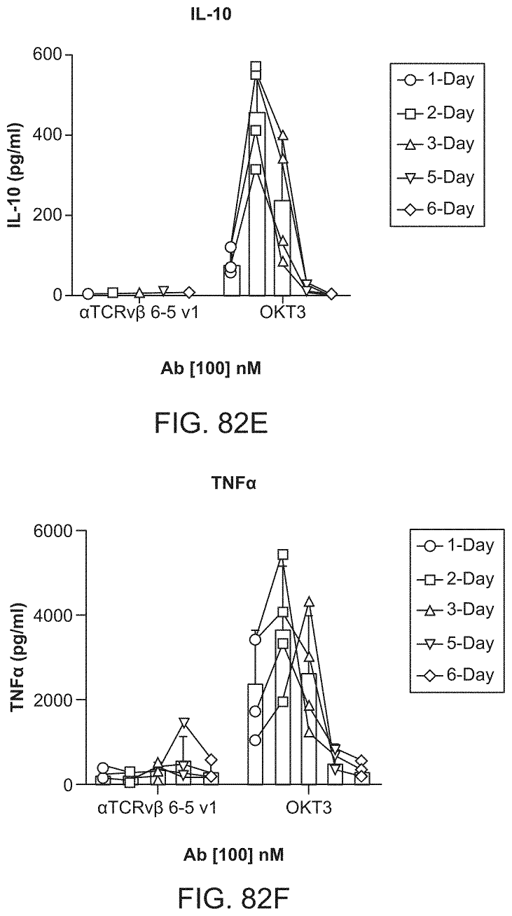

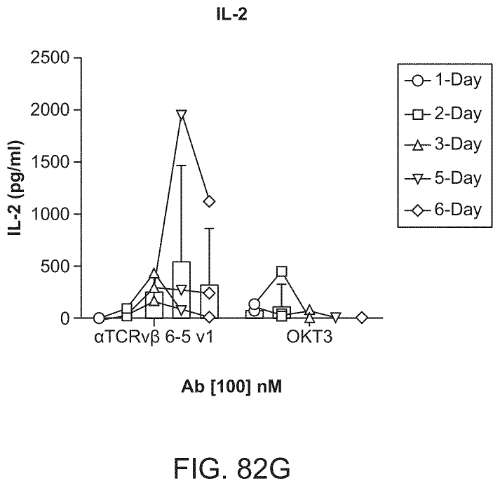

A series of experiments using the methods previously described, but varying the culture period were conducted with PBMCs from different donors. In one experiment, PBMCs from four different donors were cultured in plates coated with immobilized anti-TCRβV antibody anti-TCRβV 6-5 v1 or an anti-CD3ε antibody, either OKT3 or SP37-2 for 1-6 days. The data confirms that T cells activated/expanded with an anti-TCRβV antibody as compared to anti-CD3ε antibody release lower levels of IFNγ (FIG. 82A), IL-1β (FIG. 82B), IL-4 (FIG. 82C), IL-6 (FIG. 82D), IL10 (FIG. 82E), and TNFα (FIG. 82F); and higher levels of IL-2 (FIG. 82G).

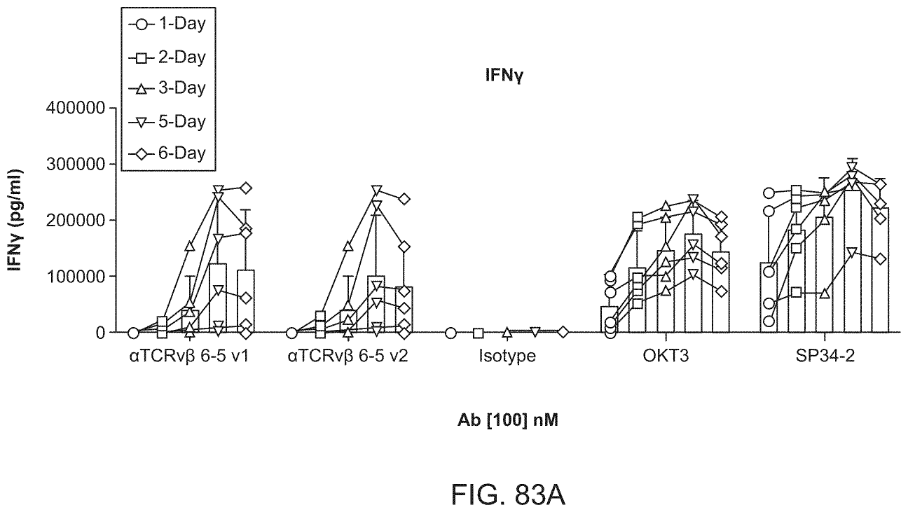

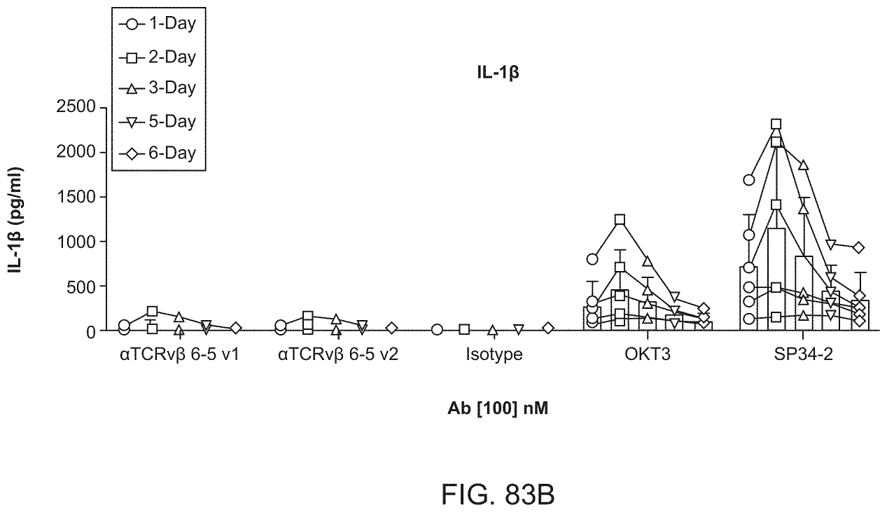

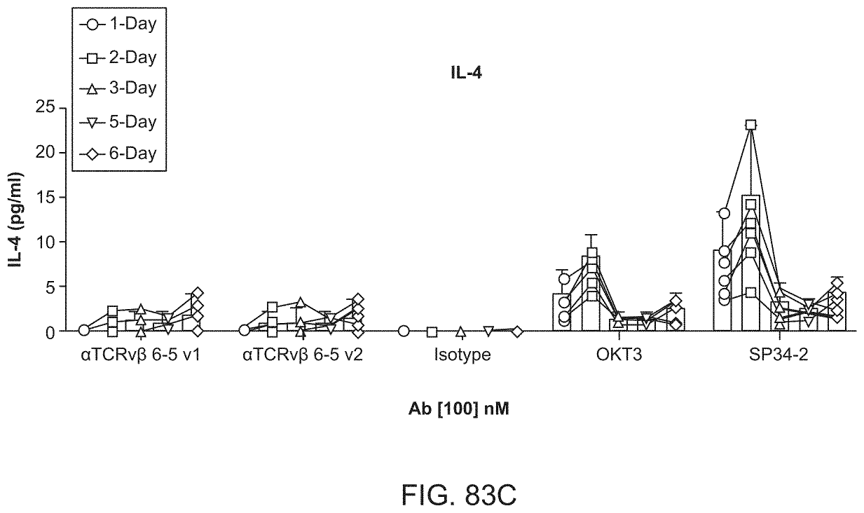

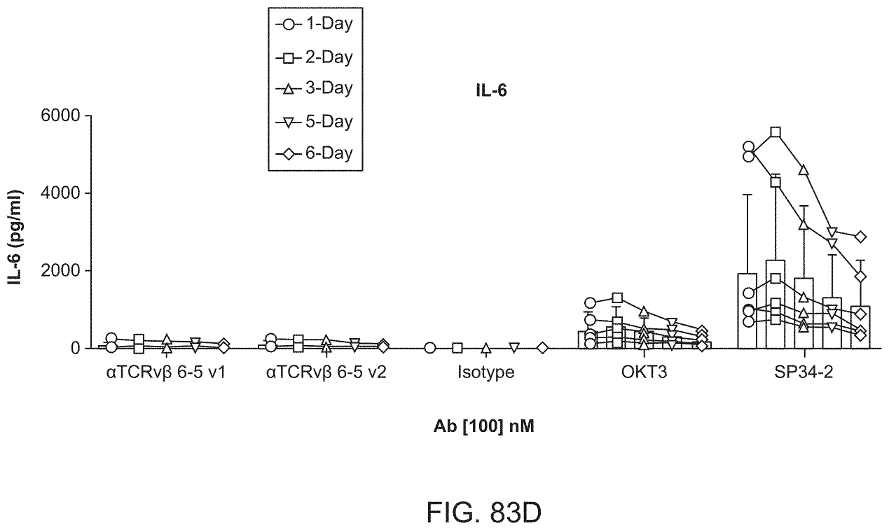

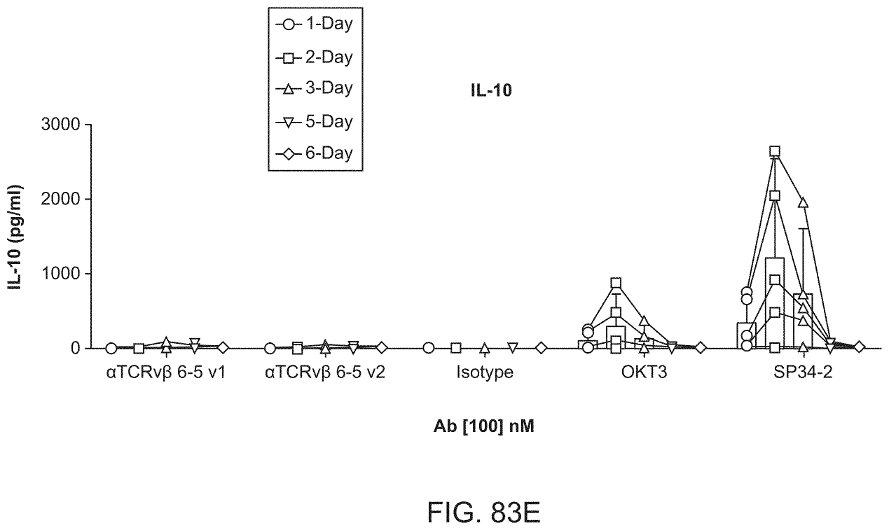

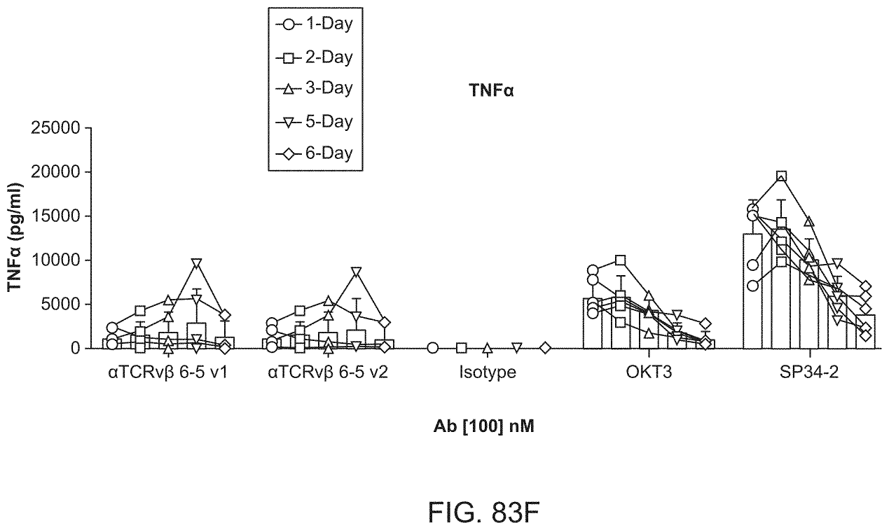

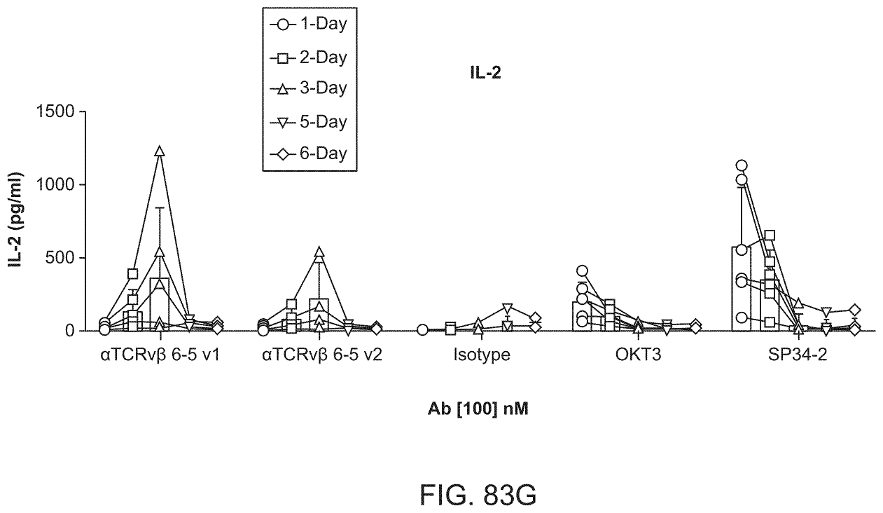

In a second experiment, PBMCs from six different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 1-6 days, or isotype control. The data confirms that T cells activated/expanded with an anti-TCRβV antibody as compared to anti-CD3ε antibody release lower levels of IFNγ (FIG. 83A), IL-1β (FIG. 83B), IL-4 (FIG. 83C), IL-6 (FIG. 83D), IL10 (FIG. 83E), and TNFα (FIG. 83F); and higher levels of IL-2 (FIG. 83G).

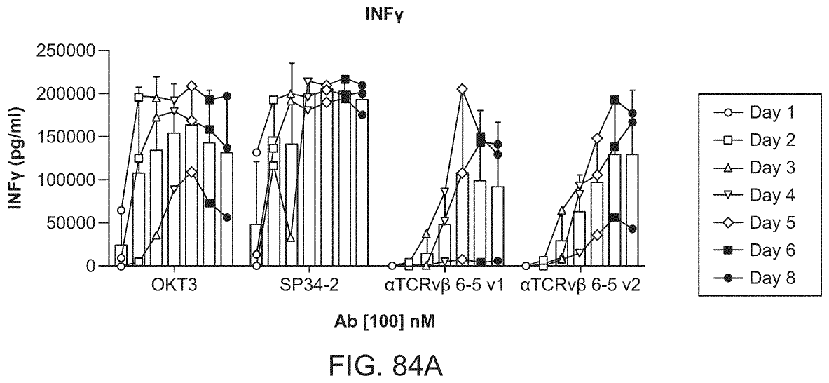

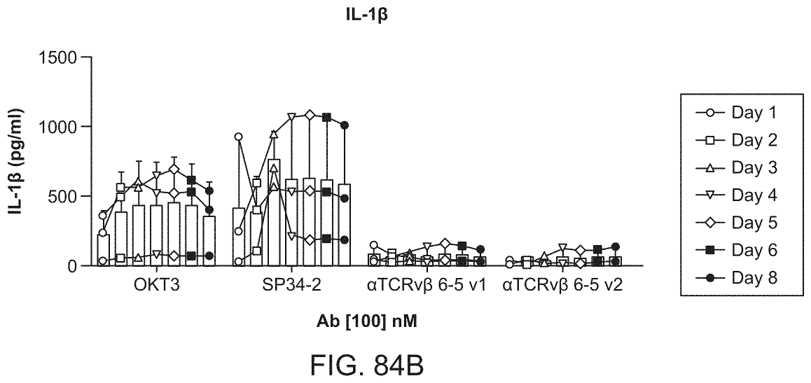

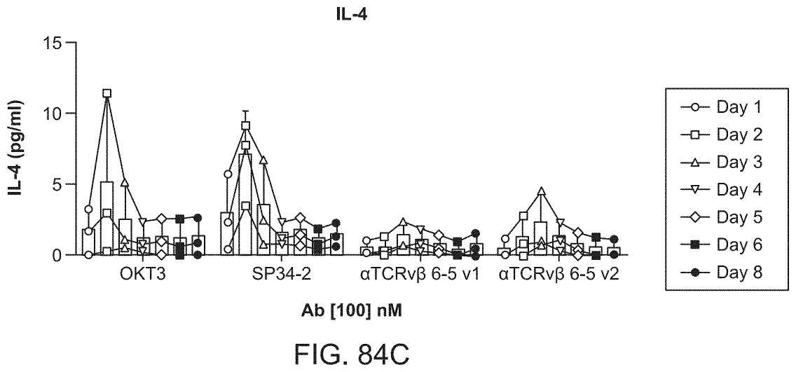

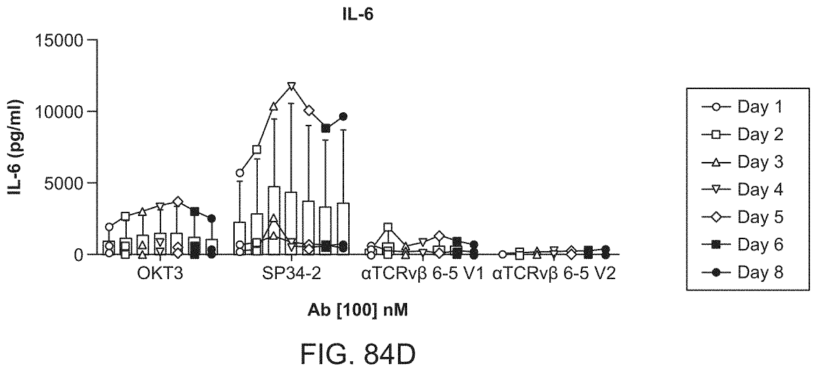

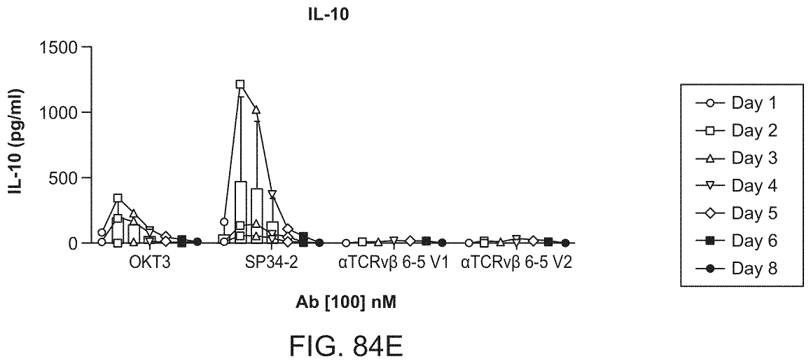

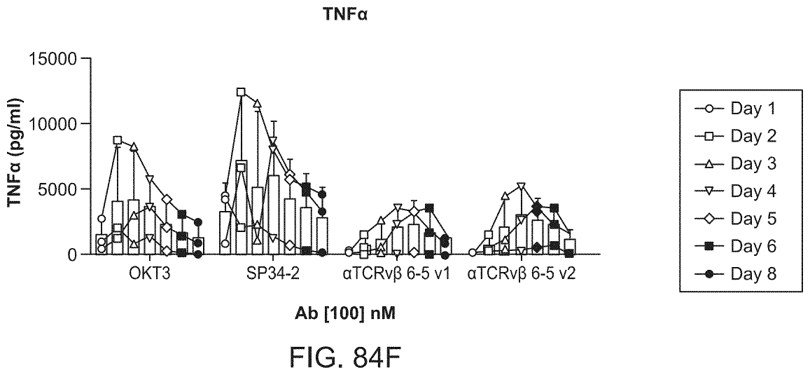

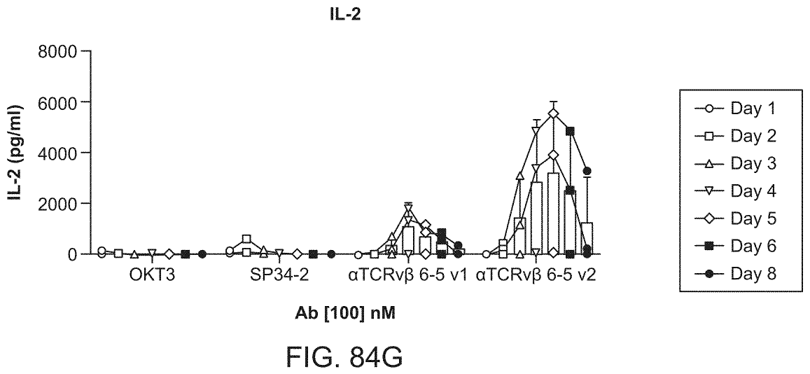

In a third experiments, PBMCs from three different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 1-8 days, or isotype control. The data confirms that T cells activated/expanded with an anti-TCRβV antibody as compared to anti-CD3ε antibody release lower levels of IFNγ (FIG. 84A), IL-1β (FIG. 84B), IL-4 (FIG. 84C), IL-6 (FIG. 84D), IL10 (FIG. 84E), and TNFα (FIG. 84F); and higher levels of IL-2 (FIG. 84G).

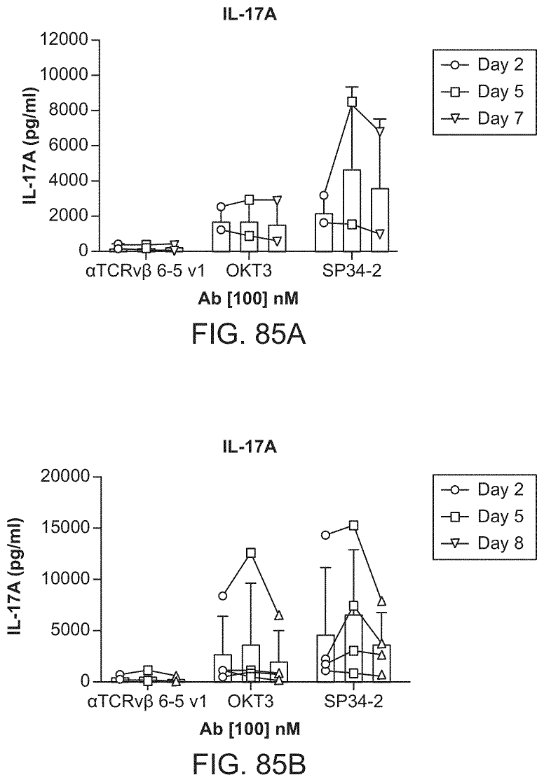

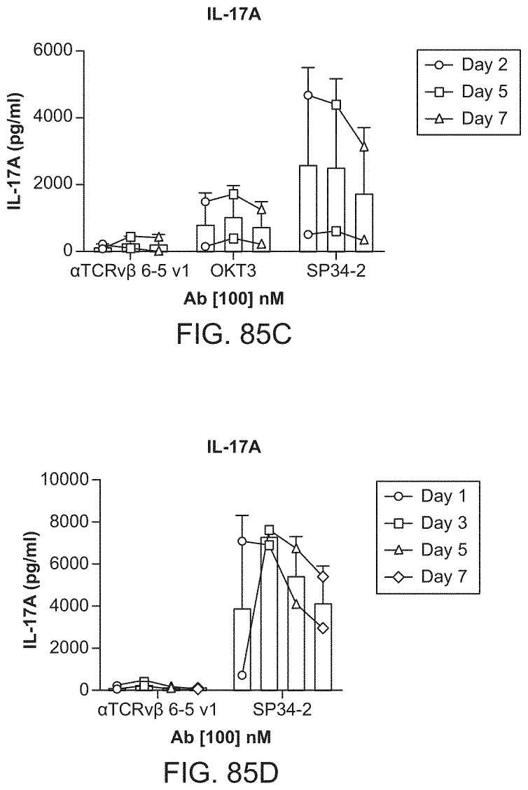

In a fourth experiments, PBMCs from two different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-7 days, or isotype control. The data confirms that T cells activated/expanded with an anti-TCRβV antibody as compared to anti-CD3ε antibody release lower levels of IL-17A (FIG. 85A). In a fifth experiments, PBMCs from four different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-8 days, or isotype control. The data confirms that T cells activated/expanded with an anti-TCRβV antibody as compared to anti-CD3ε antibody release lower levels of IL-17A (FIG. 85B). In a sixth experiments, PBMCs from two different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-7 days, or isotype control. The data confirms that T cells activated/expanded with an anti-TCRβV antibody as compared to anti-CD3ε antibody release lower levels of IL-17A (FIG. 85C). In a seventh experiments, PBMCs from two different donors were cultured in plates coated with immobilized anti-TCRβV antibody, either anti-TCRβV 6-5 v1 or anti-TCRβV 6-5 v1; or an anti-CD3ε antibody, either OKT3 or SP37-2 for 2-7 days, or isotype control. The data confirms that T cells activated/expanded with an anti-TCRβV antibody as compared to anti-CD3ε antibody release lower levels of IL-17A (FIG. 85D).

A series of similar experiments were conducted using the TCRβV antibody anti-TCRβV 6-5 v1 or anti-TCRvb 12-3/4 v1 to further assess the cytokine release profile of T cells activated/expanded using anti-TCRβV antibodies as compared to anti-CD3ε antibodies. As described above, PBMCs were cultured in cell culture plates coated with the immobilized anti-TCRβV antibody, anti-TCRβV 6-5 v1 or anti-TCRvb 12-3/4 v1; or an anti-CD3ε antibody, either OKT3 or SP37-2; isotype control; or anti-TCRβV 6-5 v1 in combination with. The cells were cultured for 1-8 days, the supernatant collected, and cytokines analyzed using Meso Scale Discovery (MSD) assay. Data generated from 2 donors and representative of 2 independent experiments.

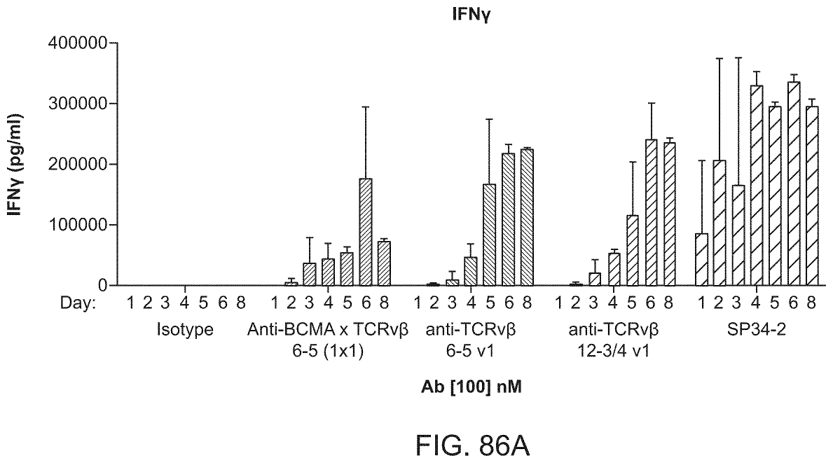

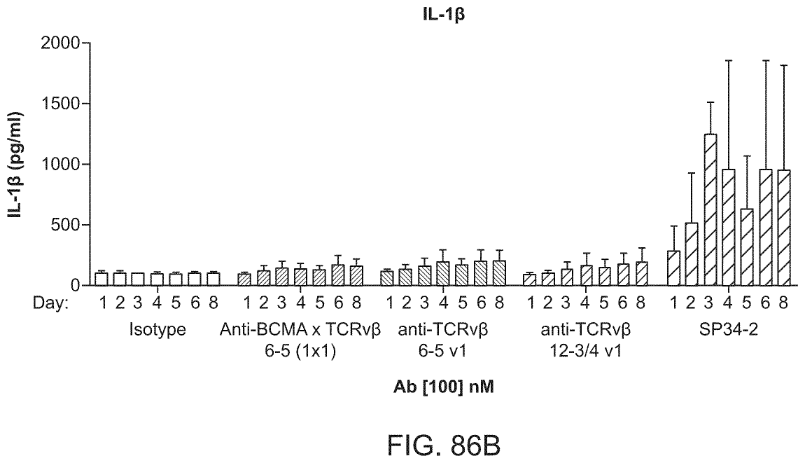

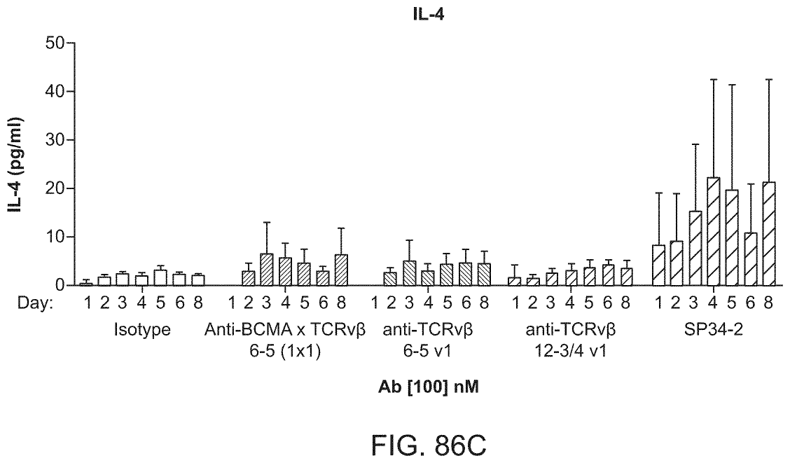

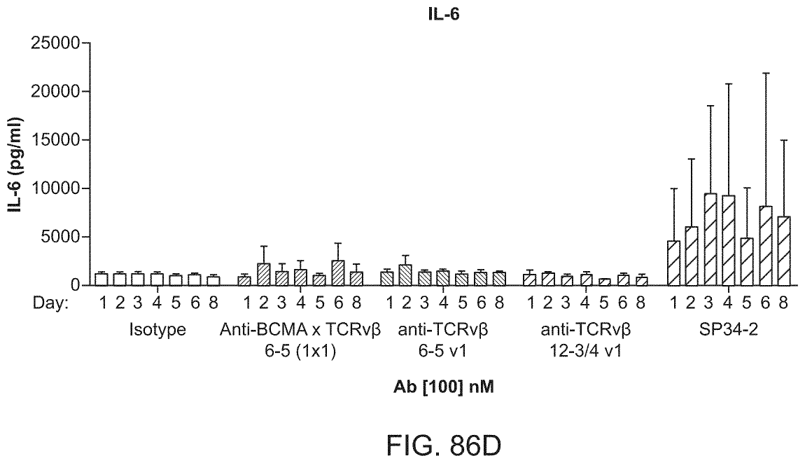

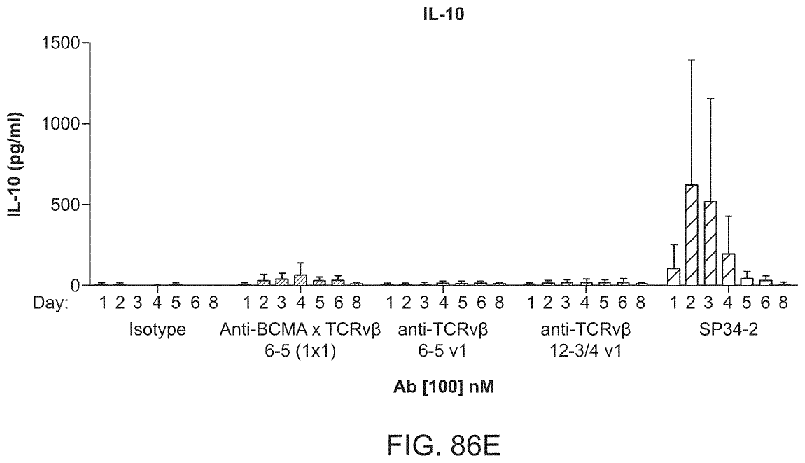

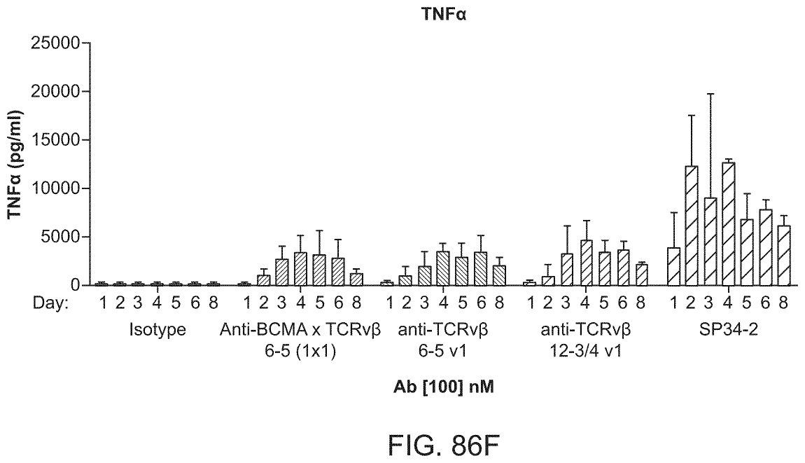

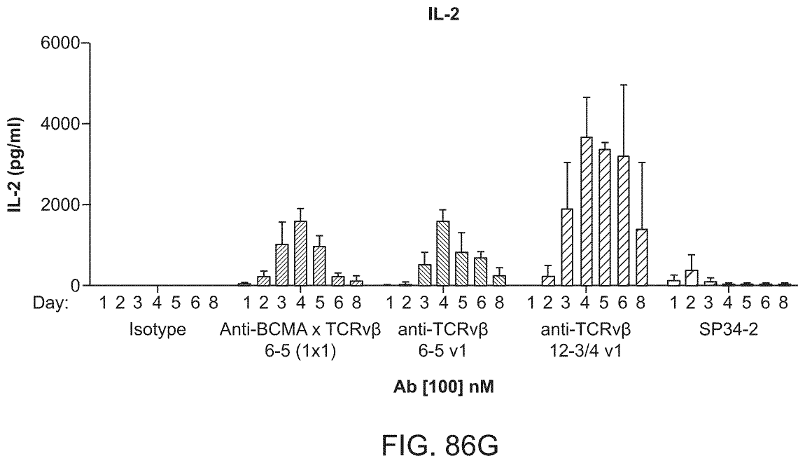

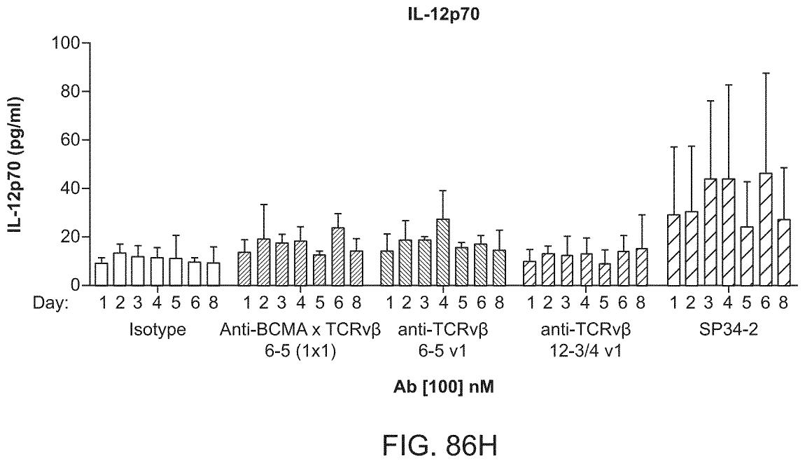

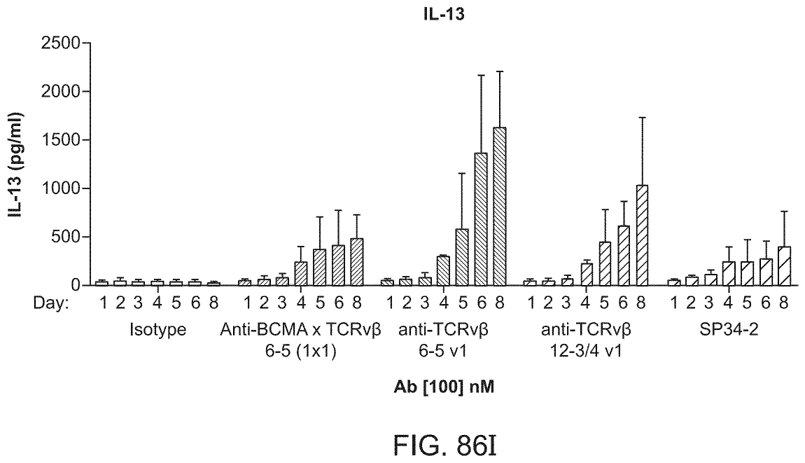

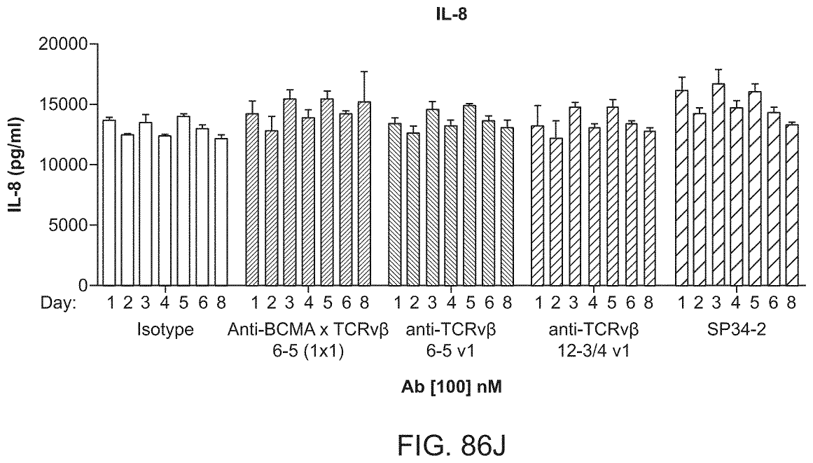

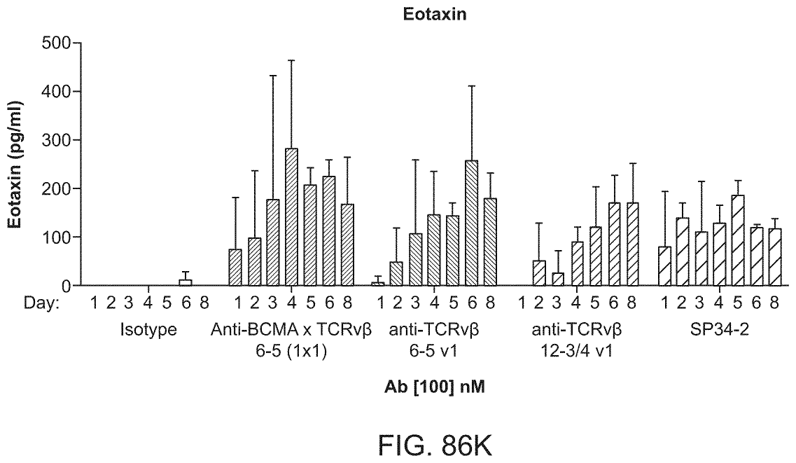

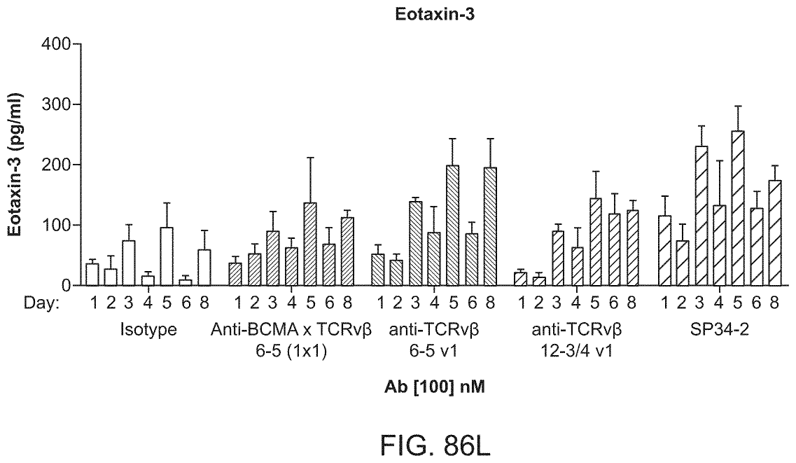

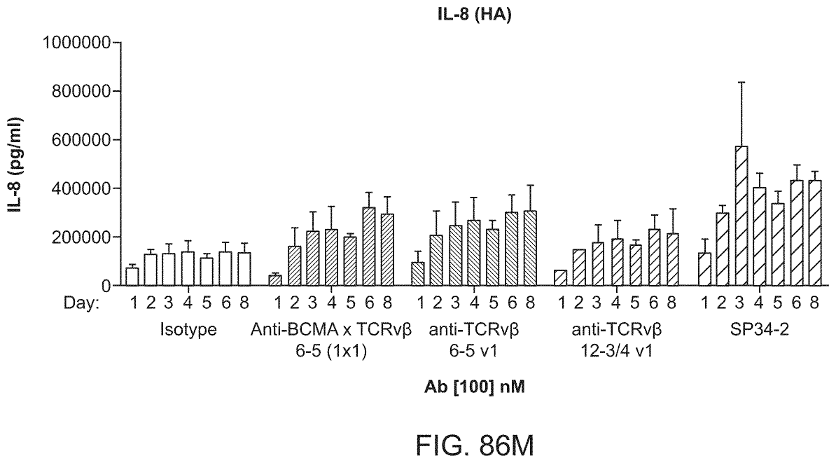

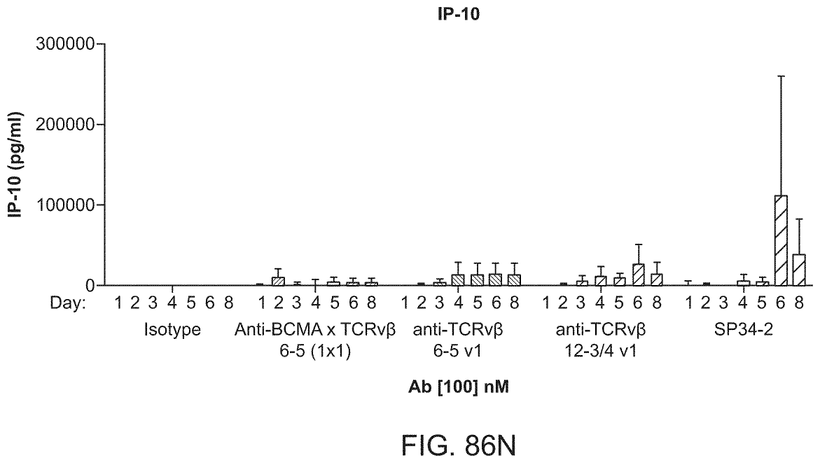

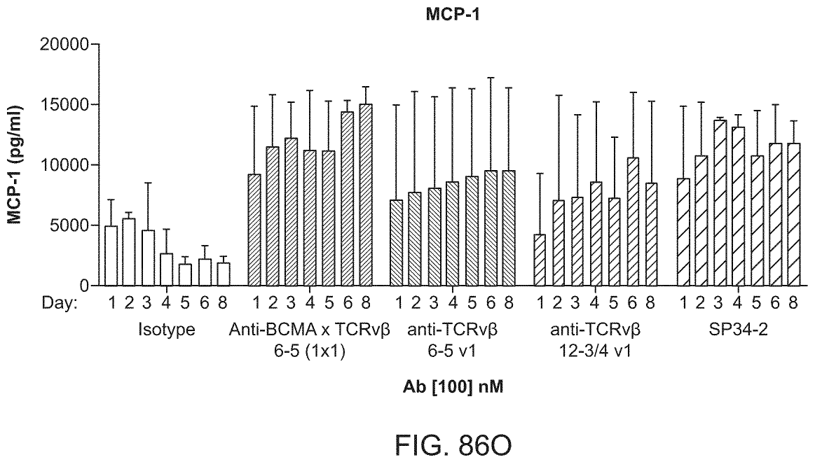

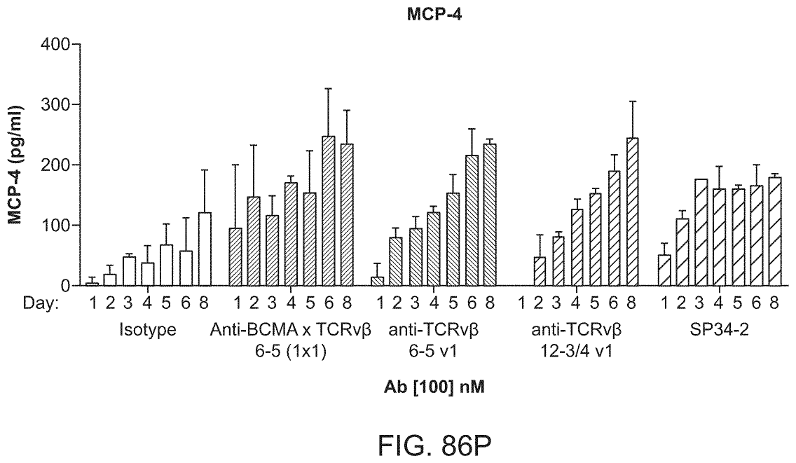

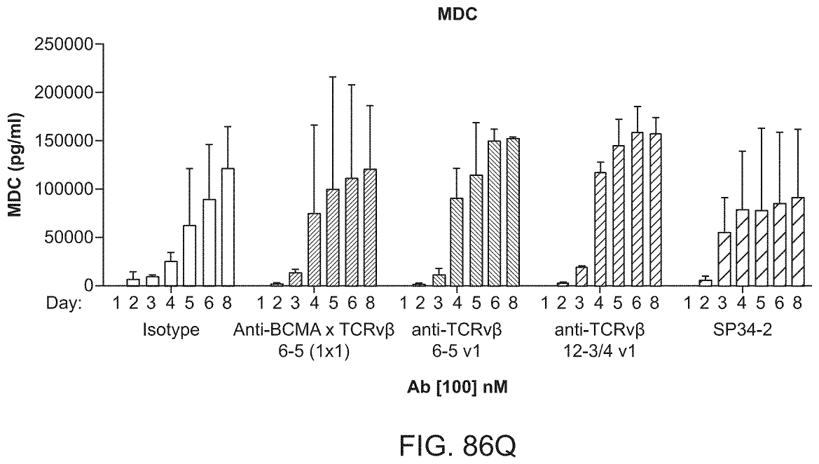

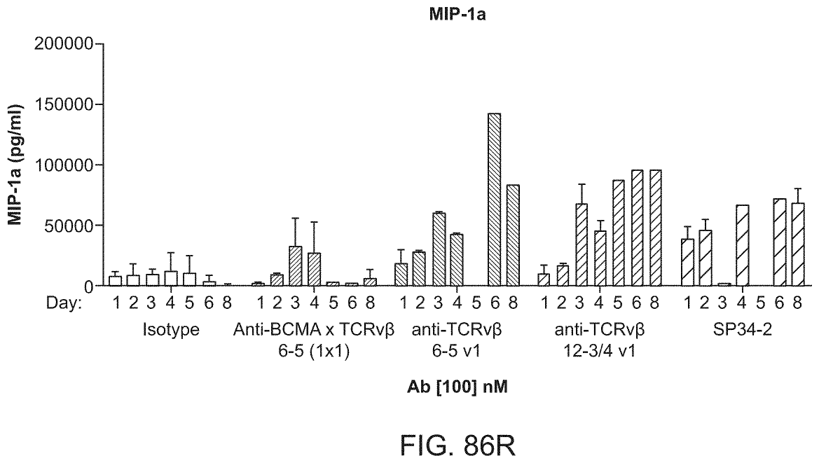

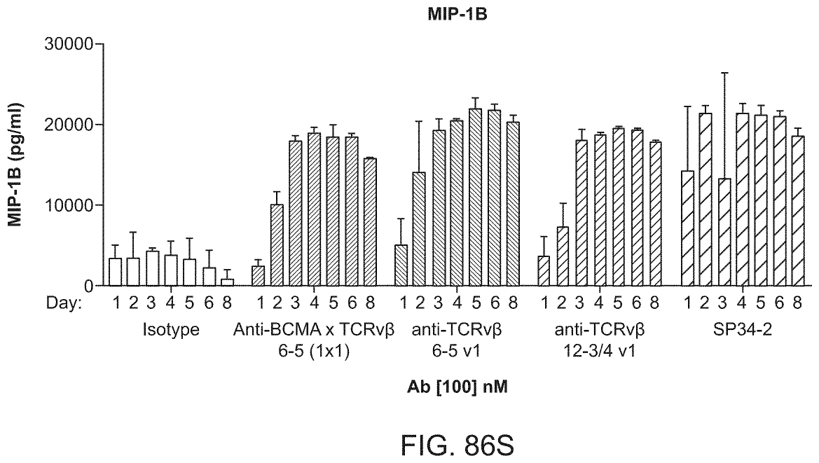

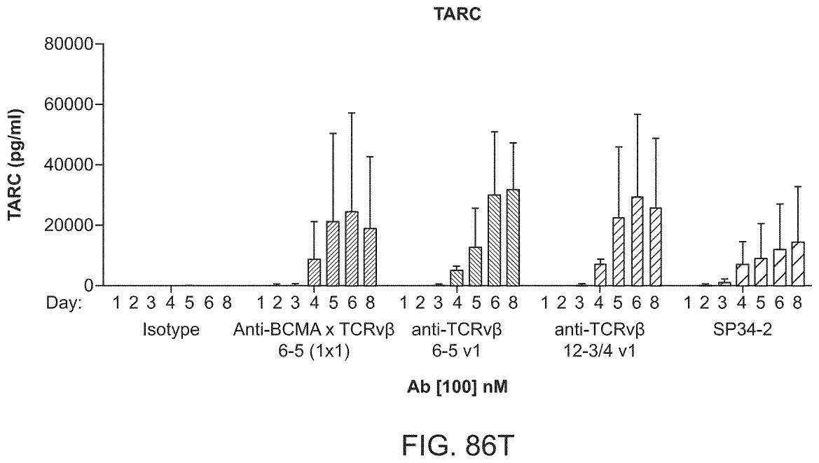

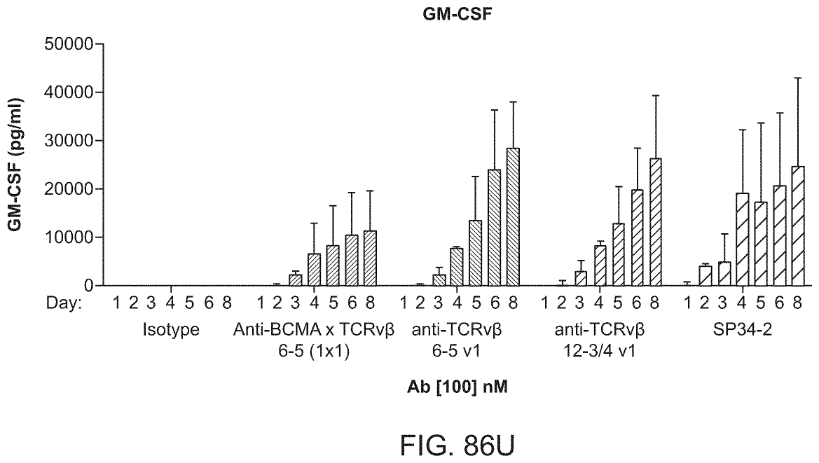

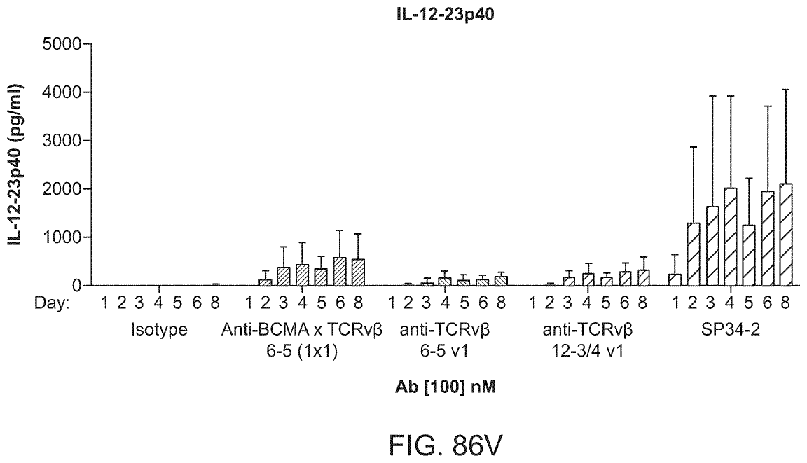

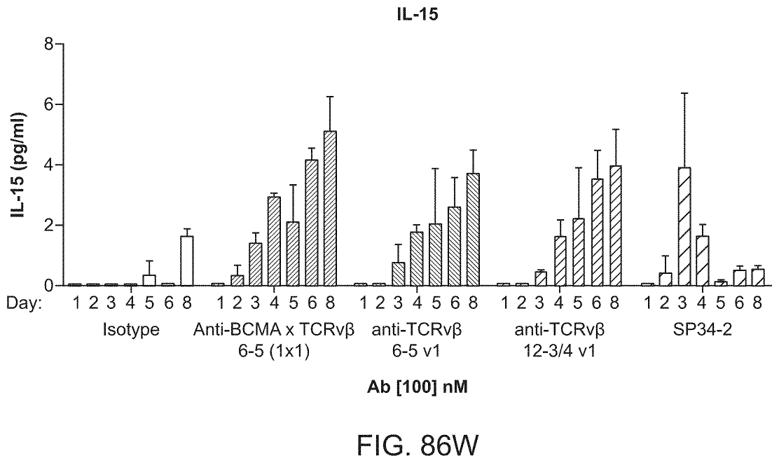

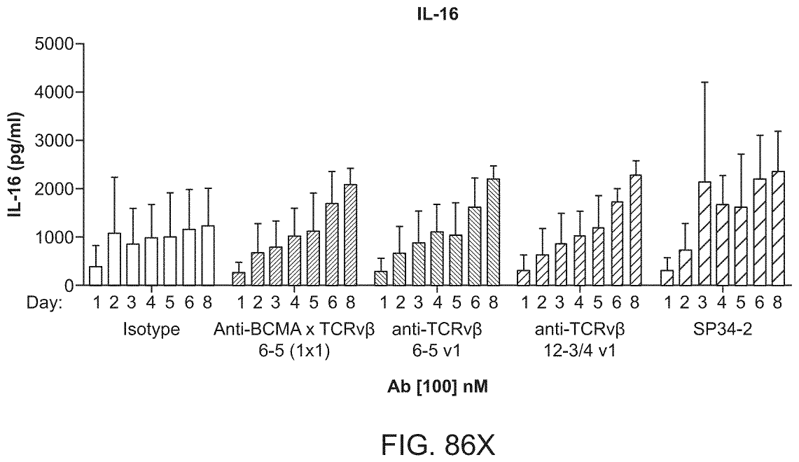

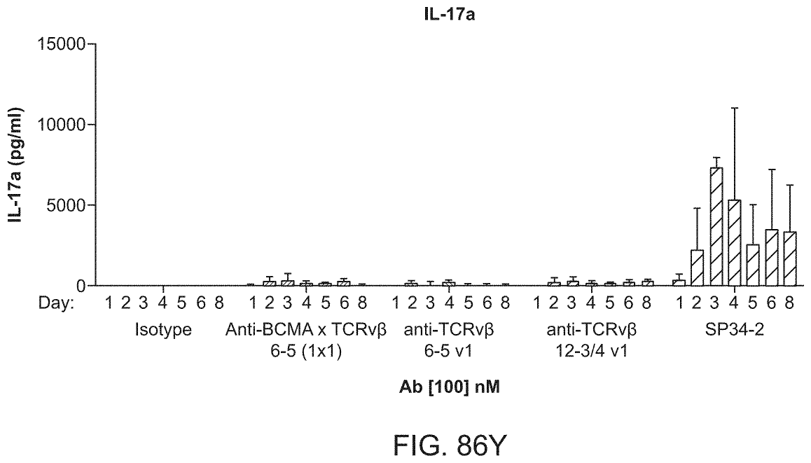

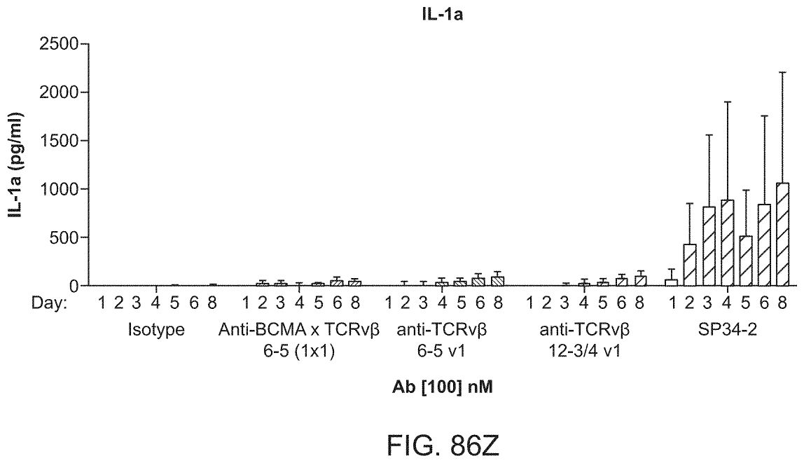

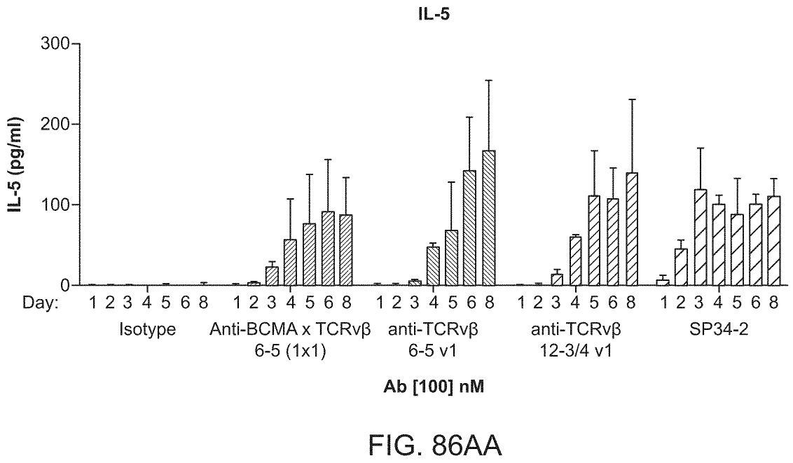

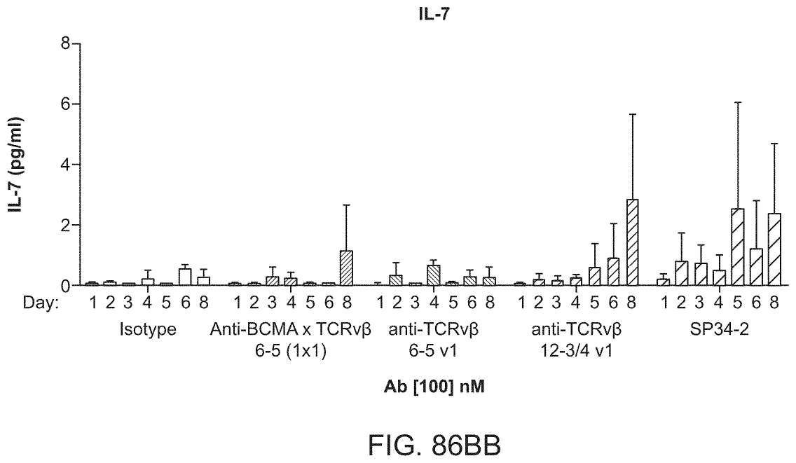

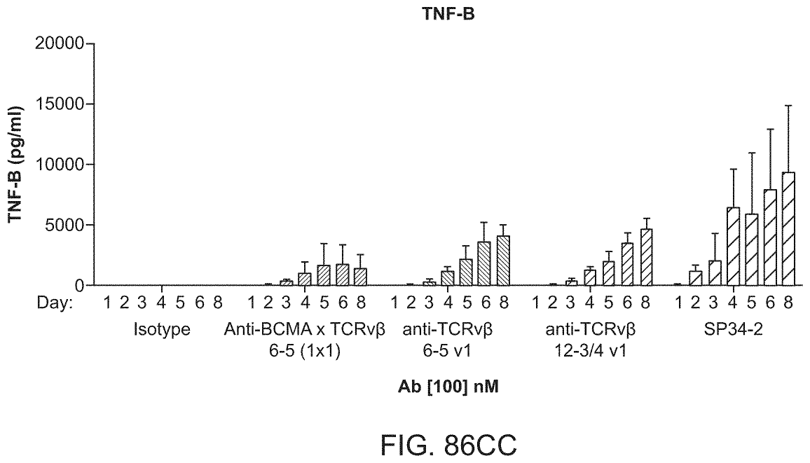

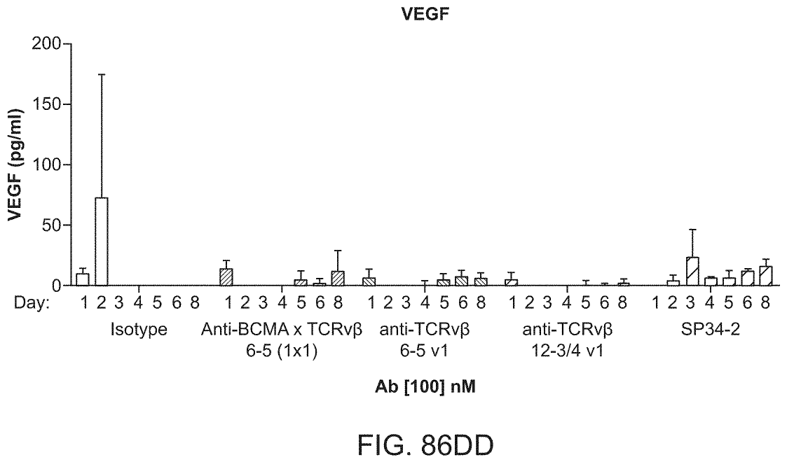

The data confirmed that T cells activated/expanded by either anti-TCRβV antibody, anti-TCRβV 6-5 v1 or anti-TCRvb 12-3/4 v1, as compared to either anti-CD3ε antibody (OKT3 or SP37-2) secreted a lower level of IFNγ (FIG. 86A), IL-1β (FIG. 86B), IL-4 (FIG. 86C), IL-6 (FIG. 86D), IL10 (FIG. 86E), TNFα (FIG. 86F); and higher levels of IL-2 (FIG. 86G). Secretion of IL-12p70 (FIG. 86H), IL-13 (FIG. 86I), IL-8 (FIG. 86J), Exotaxin (FIG. 86K), Exotaxin-3 (FIG. 86L), IL-8 (FIG. 86M), IP-10 (FIG. 86N), MCP-1 (FIG. 86O), MCP-4 (FIG. 86P), MDC (FIG. 86Q), MIP-1a (FIG. 86R), MIP-1b (FIG. 86S), TARC (FIG. 86T), GMCSF (FIG. 86U), IL-12-23p40 (FIG. 86V), IL-15 (FIG. 86W), IL-16 (FIG. 86X), IL-17a (FIG. 86Y), IL-1α (FIG. 86Z), IL-5 (FIG. 86AA), IL-7 (FIG. 86BB), TNF-β (FIG. 86CC), and VEGF (FIG. 86DD), wherein also tested.

In addition to determining the cytokine profile of T cells activated with the αTCRβV antibodies αTCRβV 6-5 v1 and αTCRβV 6-5 v2 (described above); the assays were conducted with additional αTCRβV antibodies recognizing different clonotypes.

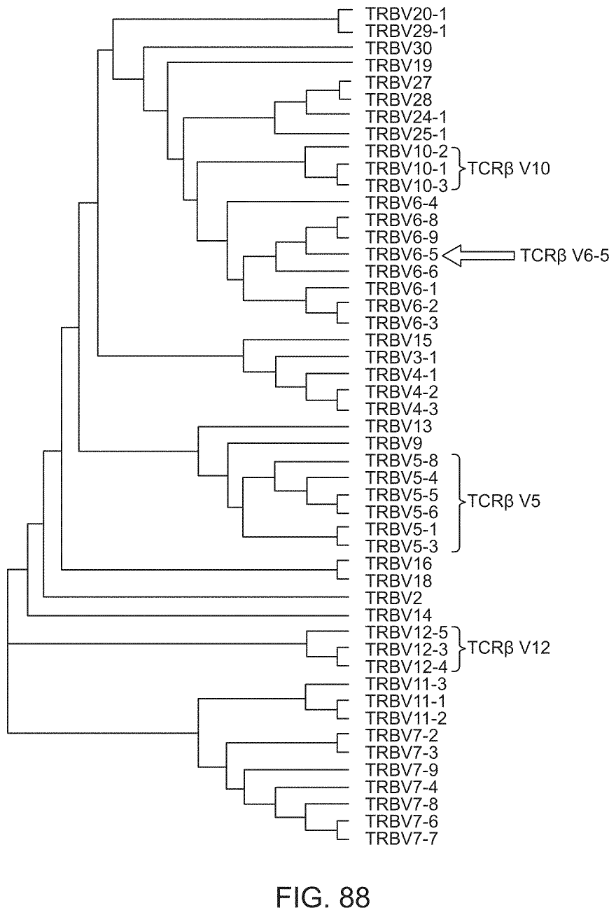

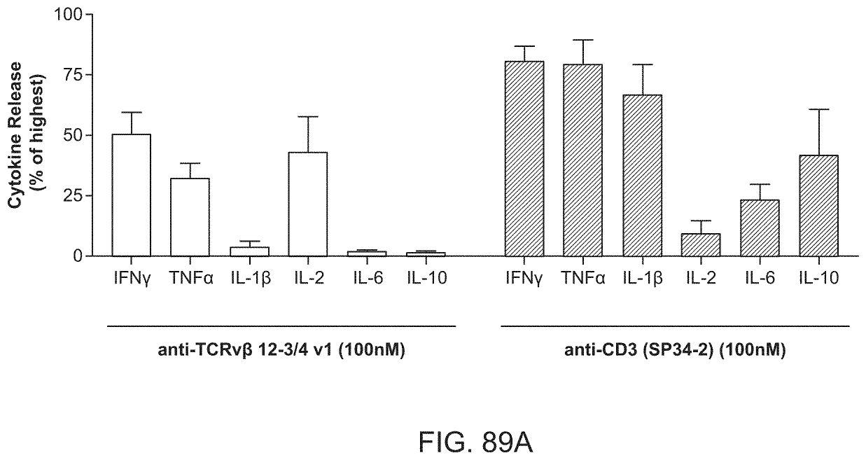

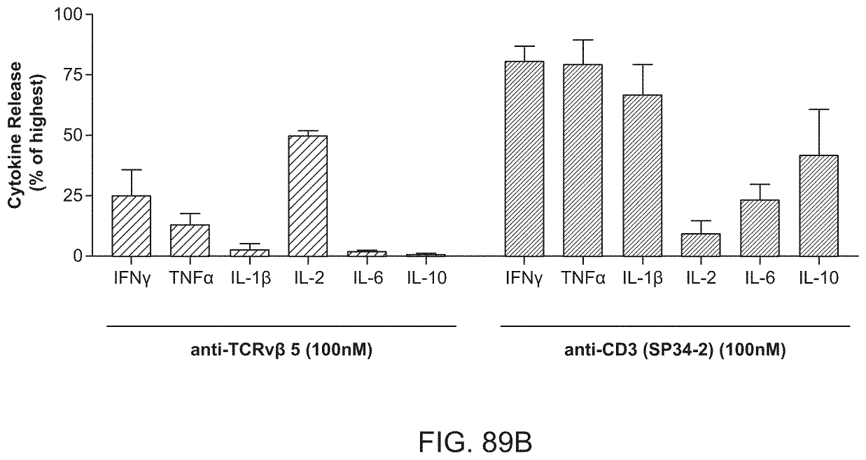

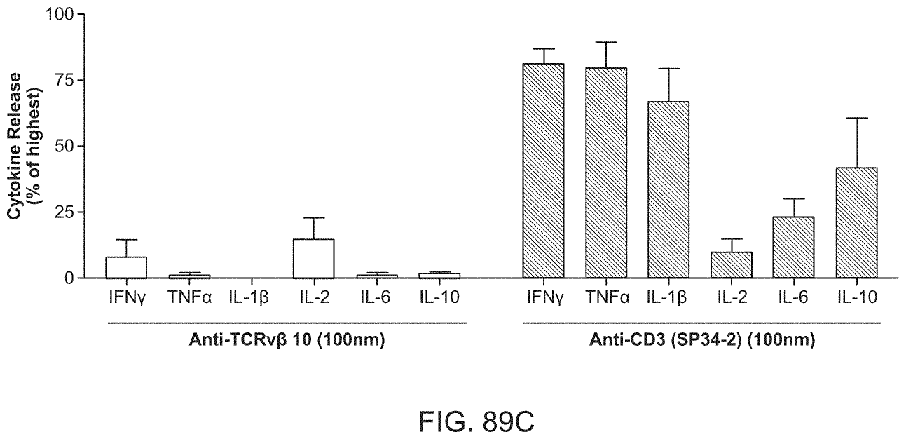

In one series of experiments antibodies tested included anti-TCRvb 12-3/4 v1, anti-TCRvb 10, and anti-TCRvb 5. Per the protocol described above, human PBMCs were solid-phase stimulated (plate-coated) with the indicated T cell-activating antibody (anti-TCRvb 12-3/4 v1, anti-TCRvb 10, anti-TCRvb 5, or the anti-CD3ε antibody SP34) at 100 nM. Supernatant were collected on day 1 to day 8; and cytokines were quantified using Meso Scale Discovery (MSD) assay. FIG. 88 provides a graphical representation of sequences between the different clonotypes, highlighting the four subfamilies tested in this series of experiments. PBMCs activated/expanded with the anti-TCRvb 12-3/4 v1 antibody (FIG. 89A), anti-TCRvb 10 antibody (FIG. 89B), or anti-TCRvb antibody (FIG. 89C) exhibited lower levels of secretion of cytokines associated with cytokine release syndrome, including IFNγ, TNFα, IL-1β, IL-2, IL-6, and IL-10, as compared to PBMCs activated/expanded with the anti-CD3ε antibody SP34-2.

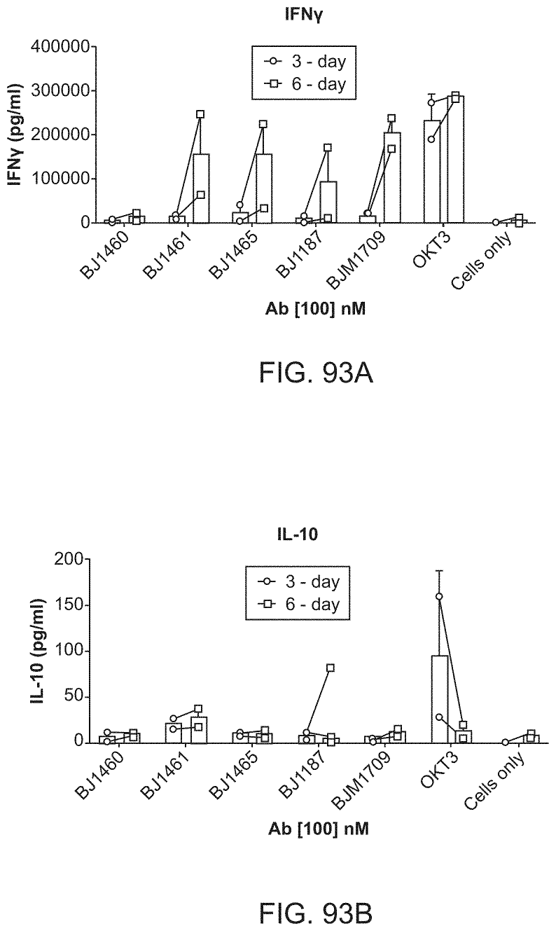

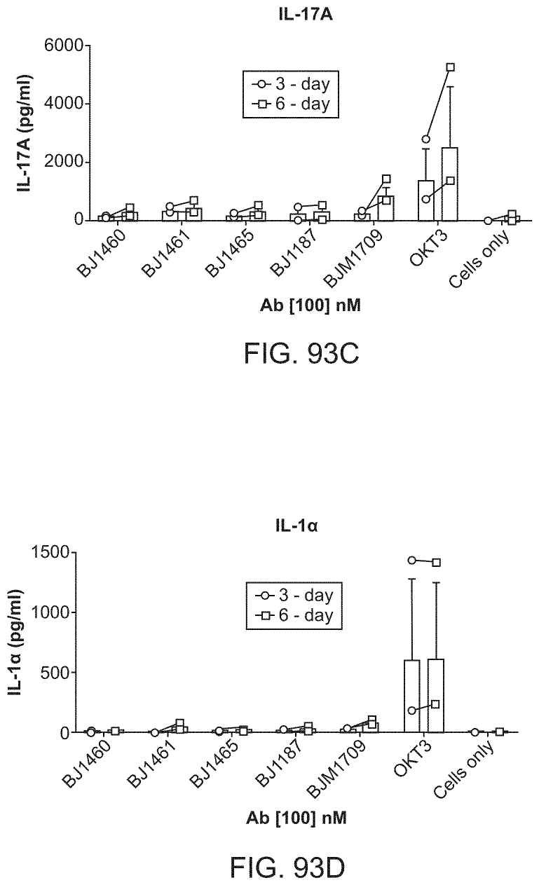

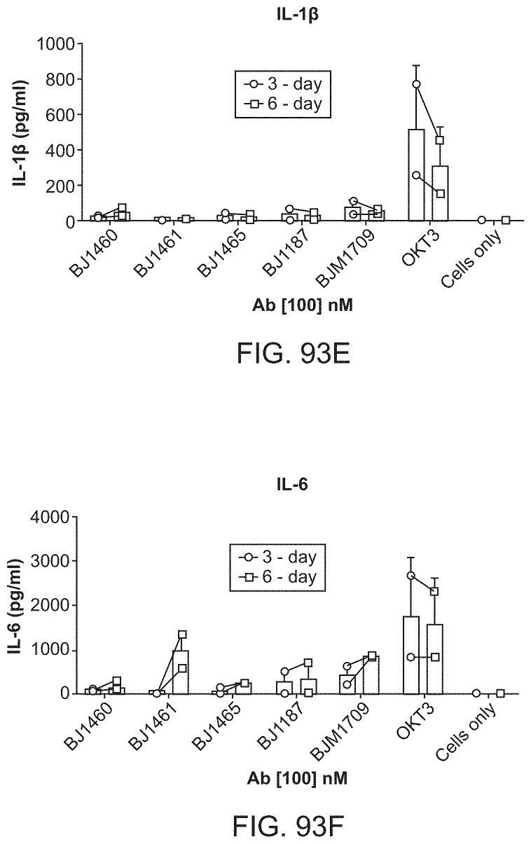

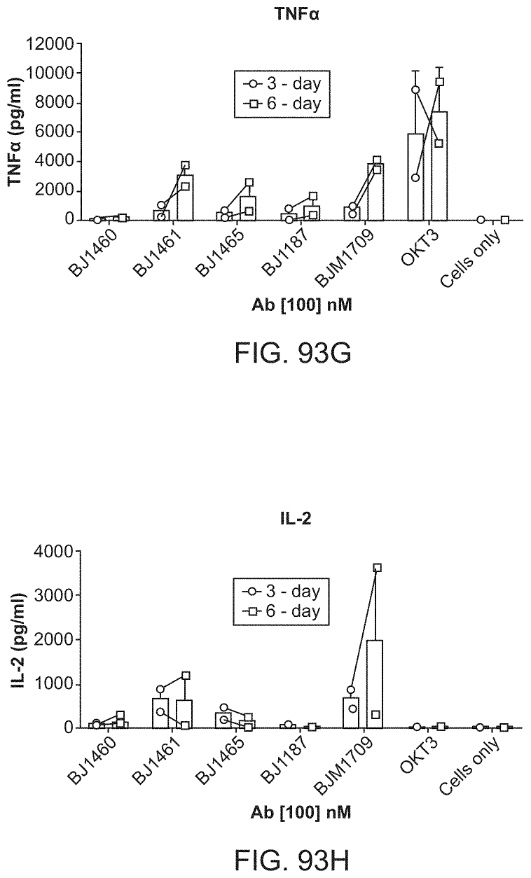

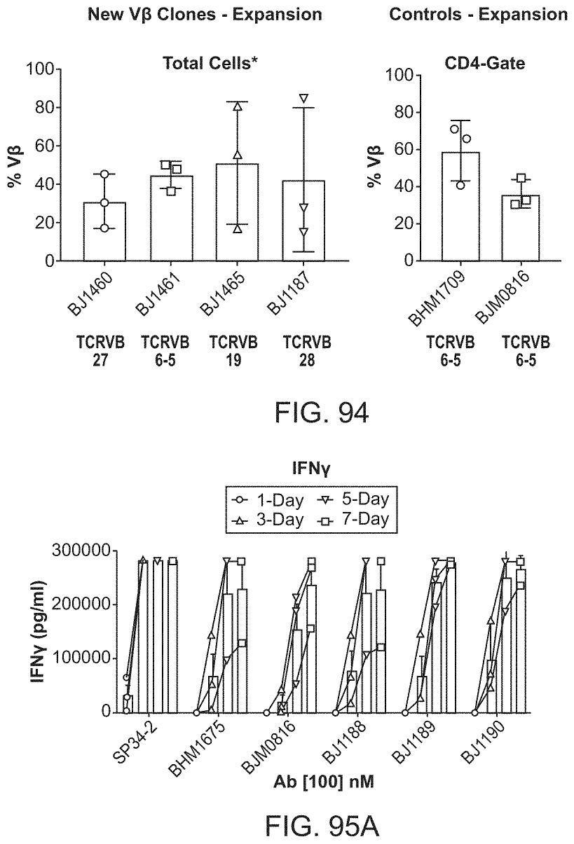

In a second series of experiments, antibodies tested included the anti-TCRVβ antibodies: BJ1460, BJ1461, BJ1465, BJ1187, BJM1709; the anti-CD3ε antibody OKT3, and a cell only control. At Day-0 PBMCs from donor 10749 were thawed and counted along with PBMCs from two fresh donors (13836 and 14828). 200,000 PBMCs in 180 uL of X-vivo media/ well (1×10e6 cells/mL) was added to a round bottom 96 well plate—one donor for ⅓ of the plate. 20 uL of 10×TCRVβ antibodies at 100 nM or 15 μg/mL were added to the wells of the plate and one triplicate of wells was added with cells only. The pate was kept in a 37° C. incubator with 5% CO₂. The cells were stimulated for 3 days with a selected antibody and 50 μL of supernatant harvested from the plate and stored at −20° C. 50 μL of media was added back to each well and the plate kept in a 37° C. incubator with 5% CO₂. On Day-6 50 uL of supernatant was harvested from each well of the plate and stored at −20° C. The cells from two wells out of the triplicate were combined and media replenished with huIL-2 was added the cell suspension for each donor was transferred into a 12-well plate. The cells were incubated overnight to allow for rest and expansion in IL-2. The cells were subsequently stained for specific Vβ-clones for detection of specific Vβ-clone expansion by FACS analysis. The concentration of cytokines (including IFNγ, IL-10, IL-17A, IL-1α, IL-1β, IL-2, IL-6, and TNFα) in the media were analyzed in the Day-3 and Day-6 supernatant samples using Meso Scale Discovery (MSD) assay. The data confirmed that PBMCs cells activated/expanded using any of the anti-TCRβV antibodies—BJ1460, BJ1461, BJ1465, BJ1187, BJM1709—secreted lower levels of IFNγ (FIG. 93A), IL-1β (FIG. 93B), IL-17A (FIG. 93C), IL-1α (FIG. 93D), IL-1β (FIG. 93E), IL-6 (FIG. 93F), TNFα (FIG. 93G); and higher levels of IL-2 (FIG. 9311). FACS analysis further showed expansion of T cells expressing the indicated TCRVβ clones (FIG. 94).

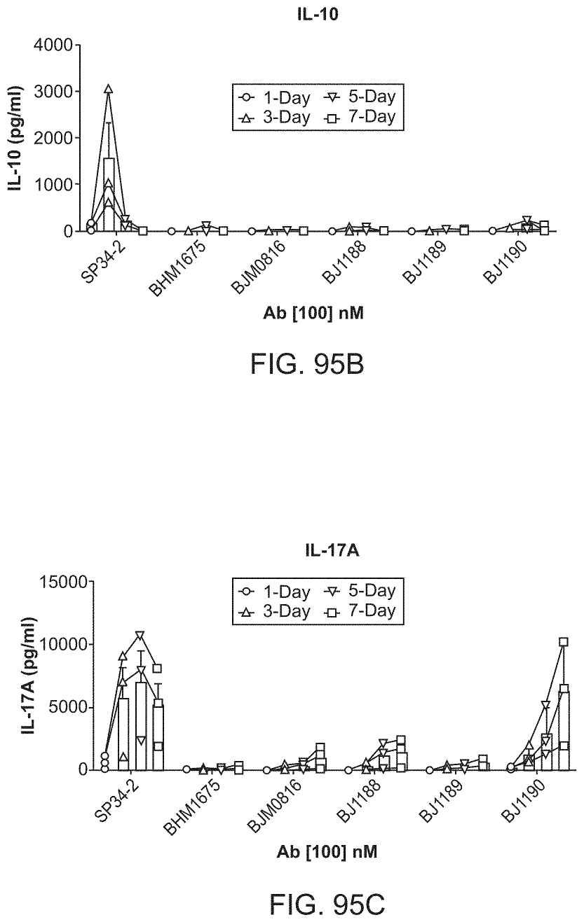

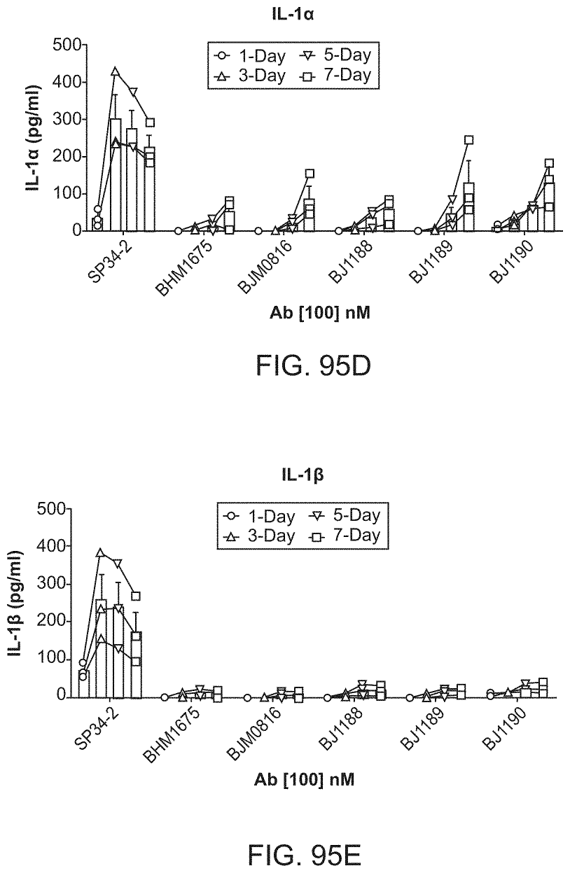

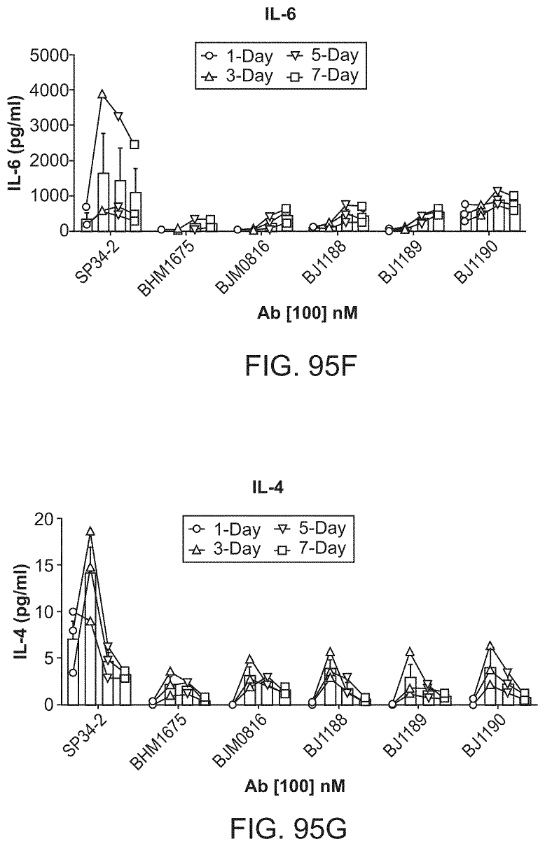

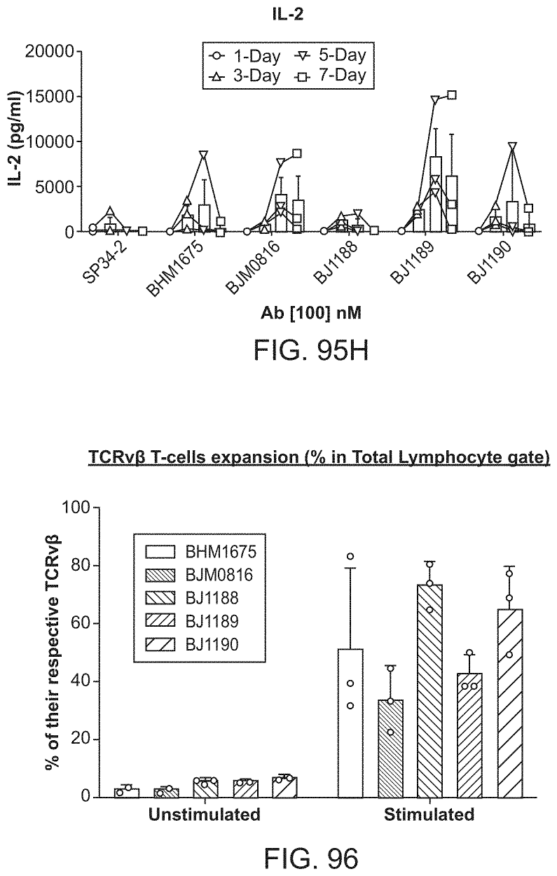

In a third series of experiments, antibodies tested included the anti-TCRVβ antibodies: BHM1675, BJM0816, BJ1188, BJ1189, BJ1190; and the anti-CD3ε antibody SP34-2. The indicated antibodies were coated into a 96-well round bottom plate at concentration of 100 nM or 15 μg/mL at 200 μl/well in PBS at 4° C. overnight or at 37° C. for a minimum of 2 hours. The plate was washed the next day with 200 μL of PBS and 0.2×10\hat{ }6 PBMCs/well from donors: CTL_123, CTL_323 and CTL_392. Supernatant samples were collected on days 1, 3, 5, and 7. A 10-plex Meso Scale Discovery (MSD) assay was run on the supernatants to determine the concentration of cytokines (including IFNγ, IL-10, IL-17A, IL-1α, IL-1β, IL-6, IL-4, and IL-2). After day 7, cells were pelleted and added to culture medium supplemented with IL-2 for one additional day to allow for expansion. Expansion of T cells expressing TCRVβ clones was analyzed by FACS staining using the same activating antibody followed by a secondary anti-human/mouse FITC antibody. Live/Dead, CD4+ and CD8+ T cells were also stained for using BHM1675, BJM0816, BJ1189 and BJ1190 antibodies. The data confirmed that PBMCs cells activated/expanded using any of the anti-TCRβV antibodies—BHM1675, BJM0816, BJ1188, BJ1189, BJ1190—secreted lower levels of IFNγ (FIG. 95A), IL-1β (FIG. 95B), IL-17A (FIG. 95C), IL-1α (FIG. 95D), IL-1β (FIG. 95E), IL-6 (FIG. 95F), IL-4 (FIG. 95G); and higher levels of IL-2 (FIG. 95H). FACS analysis further showed that TCRVβ sub-clone T-cells are expanded by their respective activation antibody (FIG. 96).

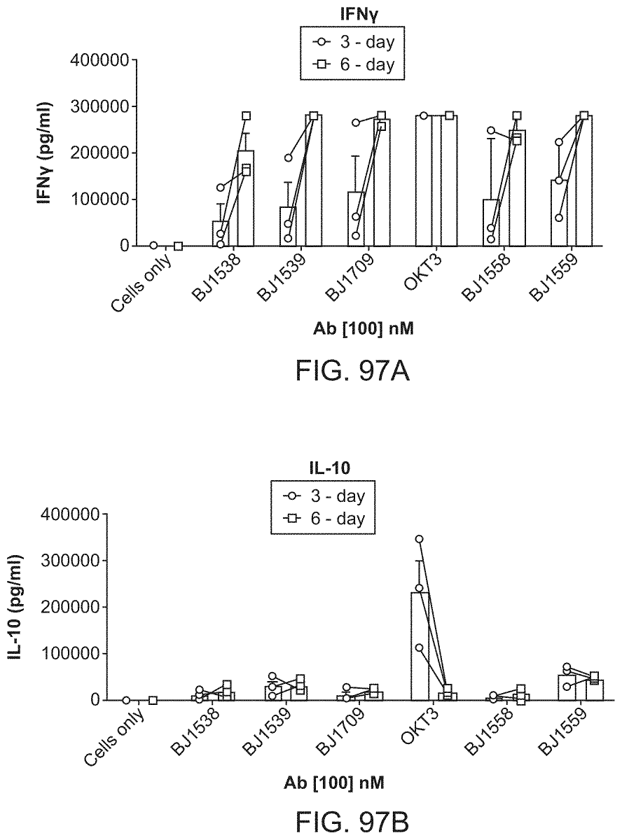

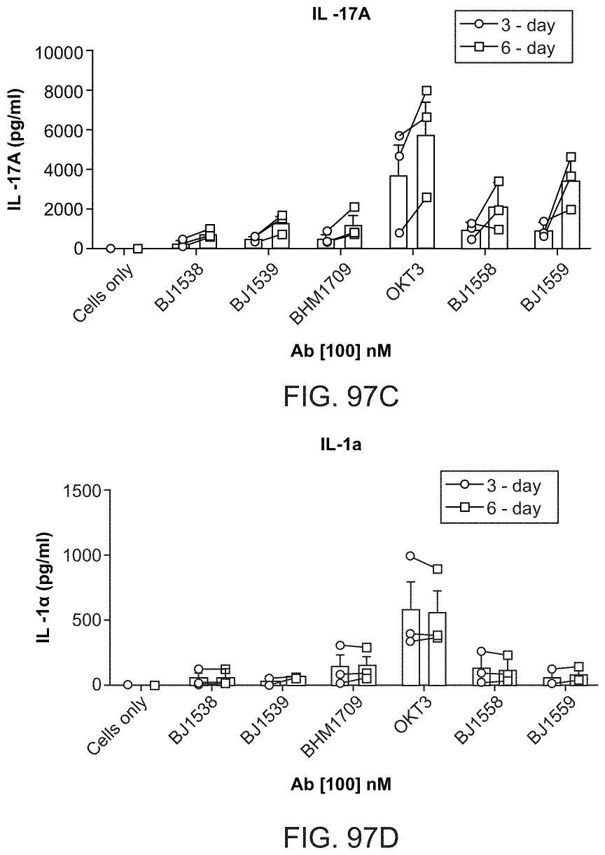

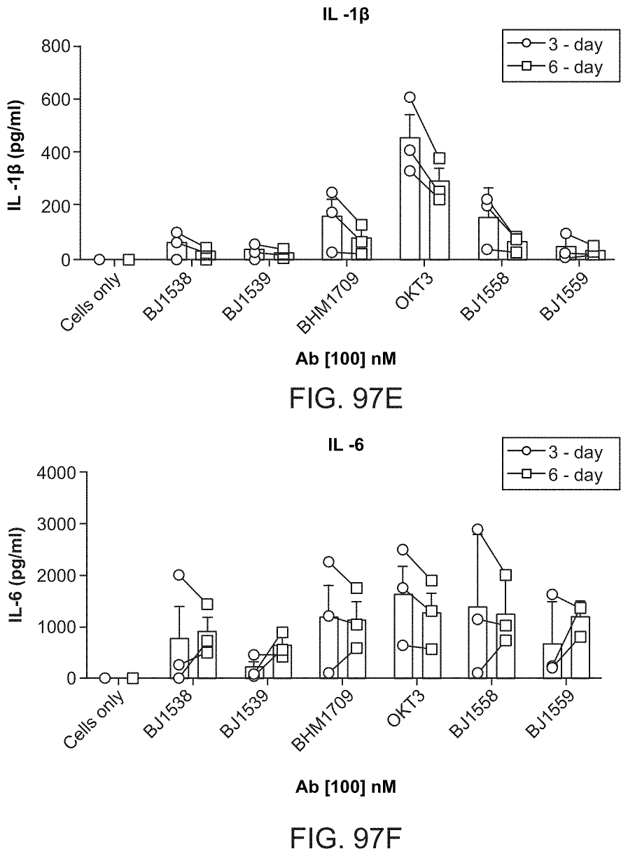

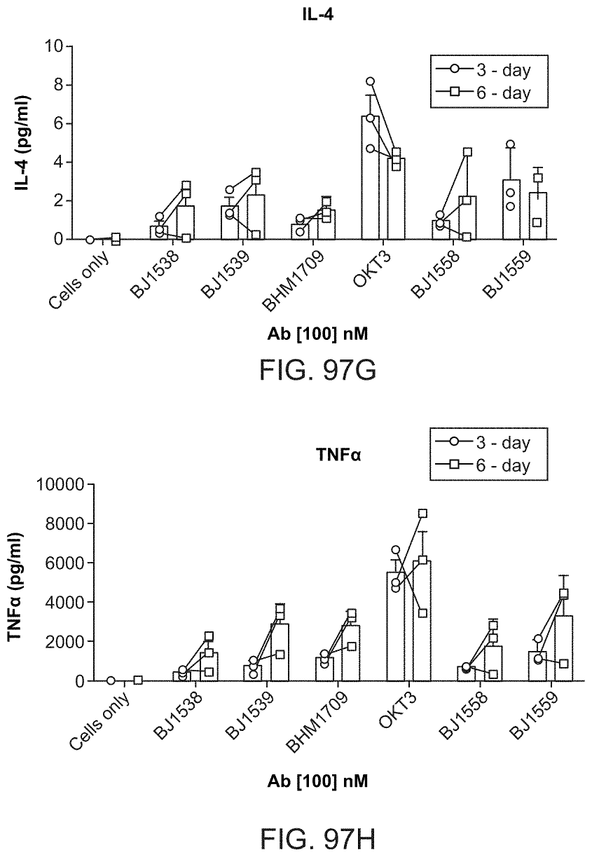

In a fourth series of experiments, antibodies tested included the anti-TCRVβ antibodies: BJ1538, BJ1539, BJ1558, BJ1559, BHM1709; and the anti-CD3ε antibody OKT3. The indicated antibodies were coated into a 96-well round bottom plate at concentration of 100 nM or 15 μg/mL at 200 μl/well in PBS at 4° C. overnight or at 37° C. for a minimum of 2 hours. The plate was washed the next day with 200 μL of PBS and 0.2×10\hat{ } PBMCs/well from donors: 10749, 5078 and 15562 (frozen and thawed samples). Supernatant samples were collected on days 3 and 6. A 10-plex Meso Scale Discovery (MSD) assay was run on the supernatants to determine the concentration of cytokines (including IFNγ, IL-10, IL-17A, IL-1α, IL-1β, IL-6, IL-4, TNFα, and IL-2). The data confirmed that PBMCs cells activated/expanded using any of the anti-TCRβV antibodies—BJ1538, BJ1539, BJ1558, BJ1559, BHM1709—secreted lower levels of IFNγ (FIG. 97A), IL-1β (FIG. 97B), IL-17A (FIG. 97C), IL-1α (FIG. 97D), IL-1β (FIG. 97E), IL-6 (FIG. 97F), IL-4 (FIG. 97G) TNFα (FIG. 97H); and higher levels of IL-2 (FIG. 97I).

In summary, the data shows that anti-TCRvb antibodies recognizing different TCRvb subtypes do not induce cytokines associated with CRS.

### Example 18: Anti-TCRvb does not Activate T Cells without Cross-Linking

To assess whether bivalent anti-TCRvb antibodies activate T cells without cross-linking—purified T cells from 2 donors were stimulated with anti-TCRvb (TCRvb 6-5 v1) or anti-CD3e (SP34), either plate-coated or in solution. Supernatants were collected at day 1, 3, 5 and 7 post activation. Cytokine secretion was detected using MSD 10 plex kit (IFN-g, IL-10, IL-15, IL-17A, IL-1a, IL-1b, IL-2, IL-4, IL-6 and TNF-α).

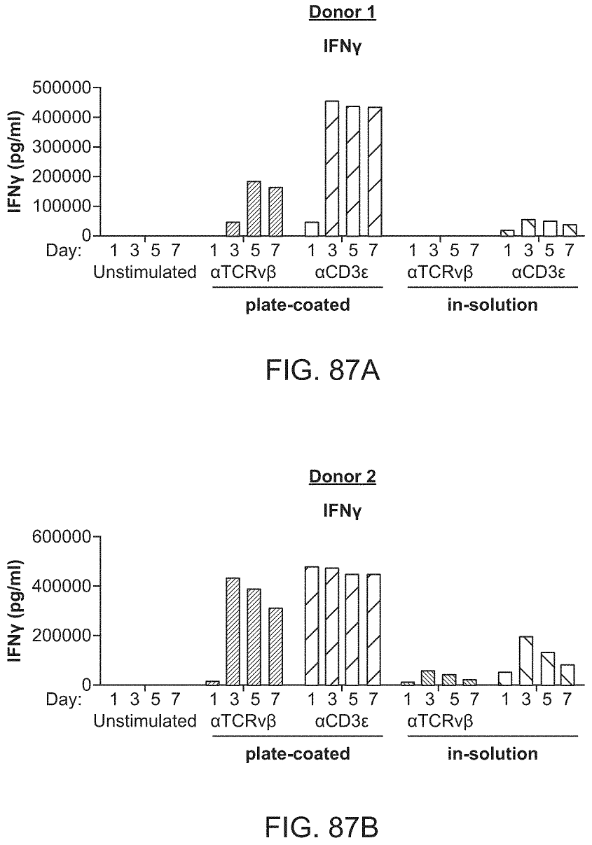

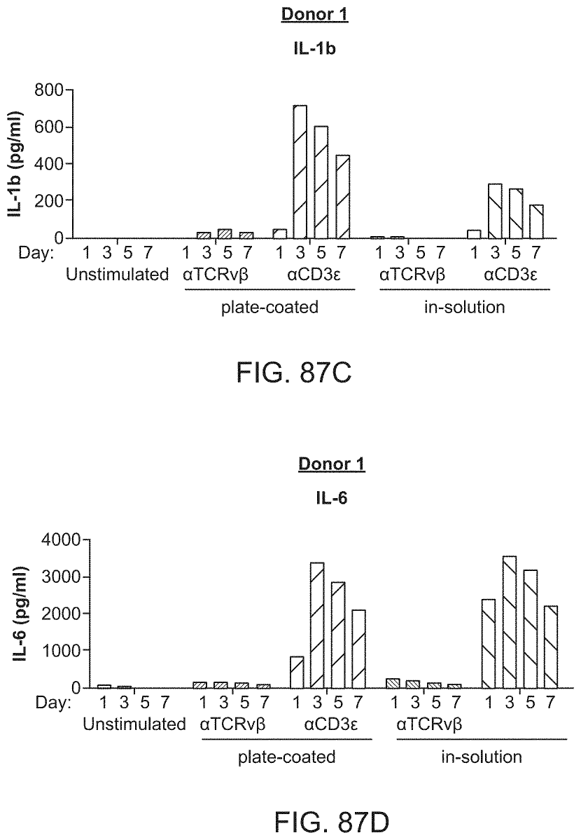

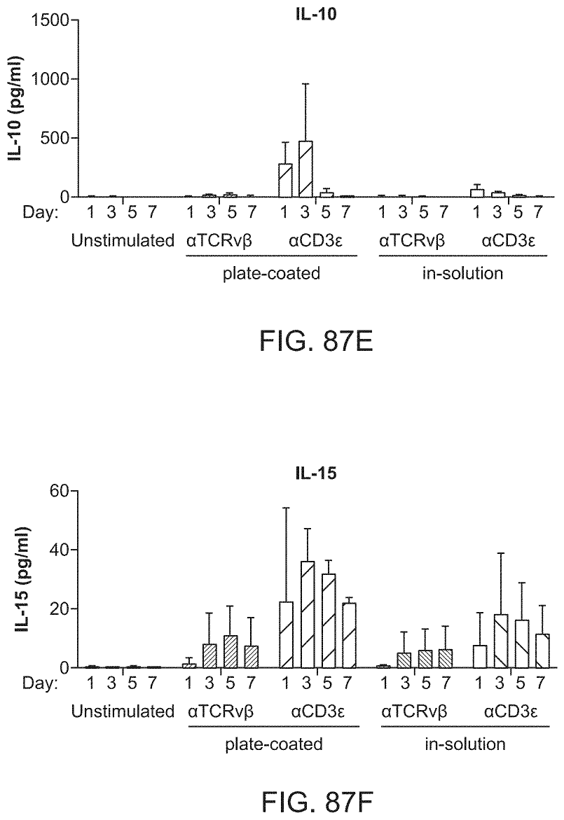

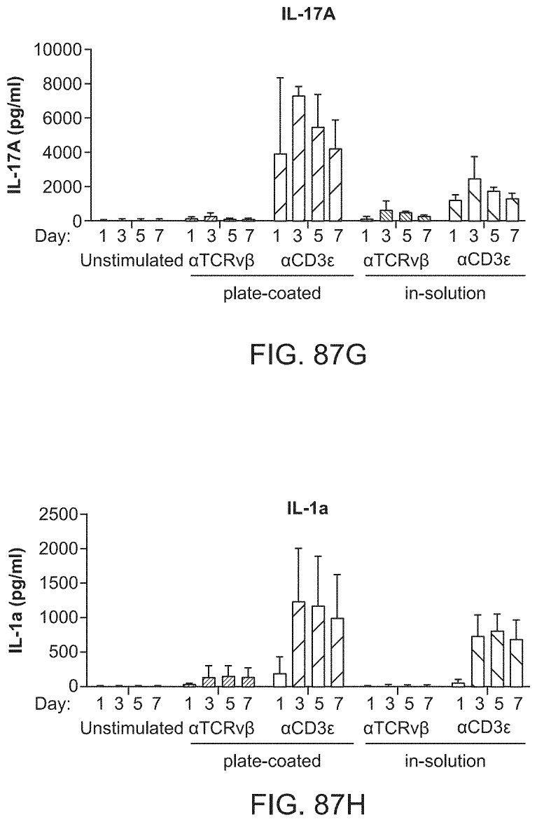

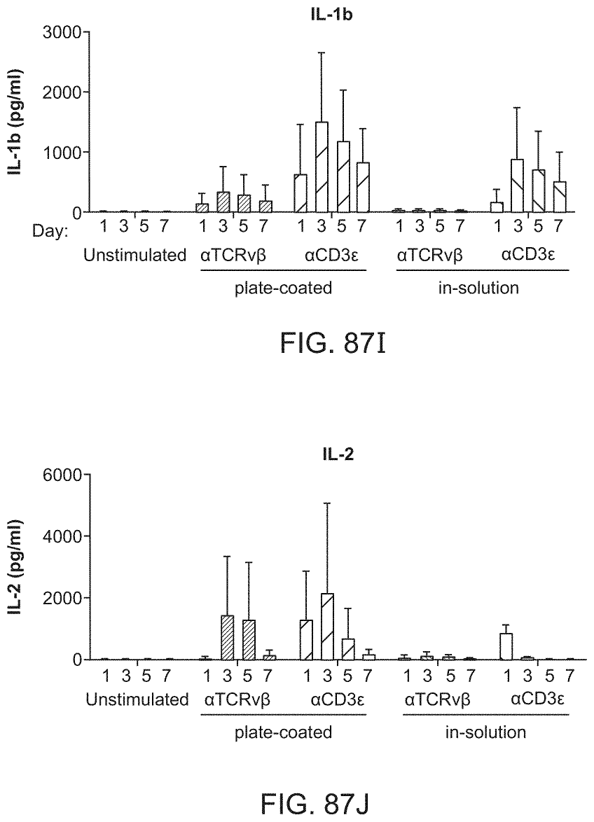

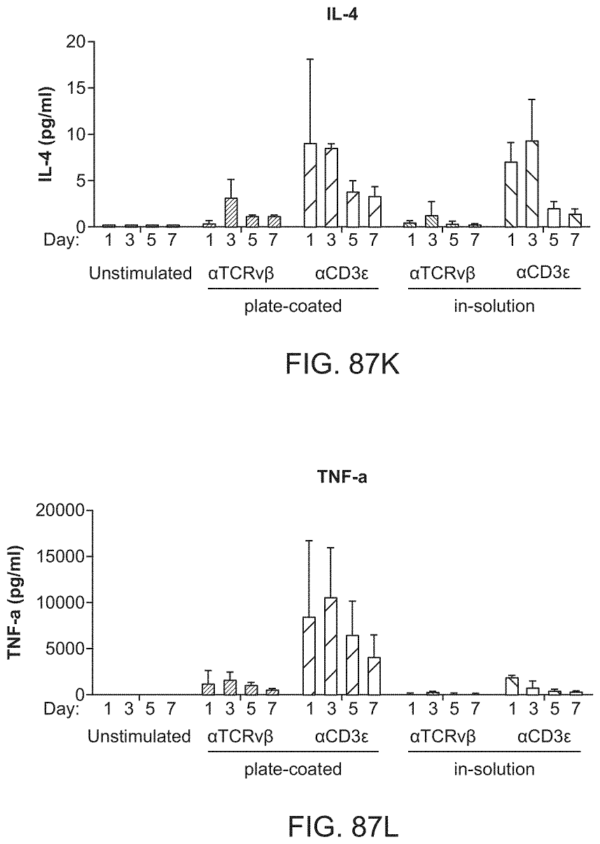

The results show the PBMCs activated/expanded with anti-TCRvb 6-5 v1 antibody in solution do no induce very little IFNγ secretion as compared to PBMCs activated/expanded with anti-TCRvb 6-5 v1 antibody in immobilized (allowing for crosslinking) (FIG. 87A and FIG. 87B). The results show the PBMCs activated/expanded with anti-TCRvb 6-5 v1 antibody in solution do no induce very little or no IL-1b (FIG. 87C and FIG. 87D), IL-10 (FIG. 87E), IL-15 (FIG. 87F), IL-17A (FIG. 87G), IL-1a (FIG. 87H), IL-1b (FIG. 87I), IL-2 (FIG. 87J), IL-4 (FIG. 87K), IL-6 (FIG. 87L), and TNF-α (FIG. 87M) secretion. In summary, the data shows that anti-CD3ε activates T cells in solution (without crosslinking); while the anti-TCRvb antibodies does not activate T-cells in solution.

### Example 19: Anti-TCRVβ 5-5, 5-6 Antibodies Compete for Binding

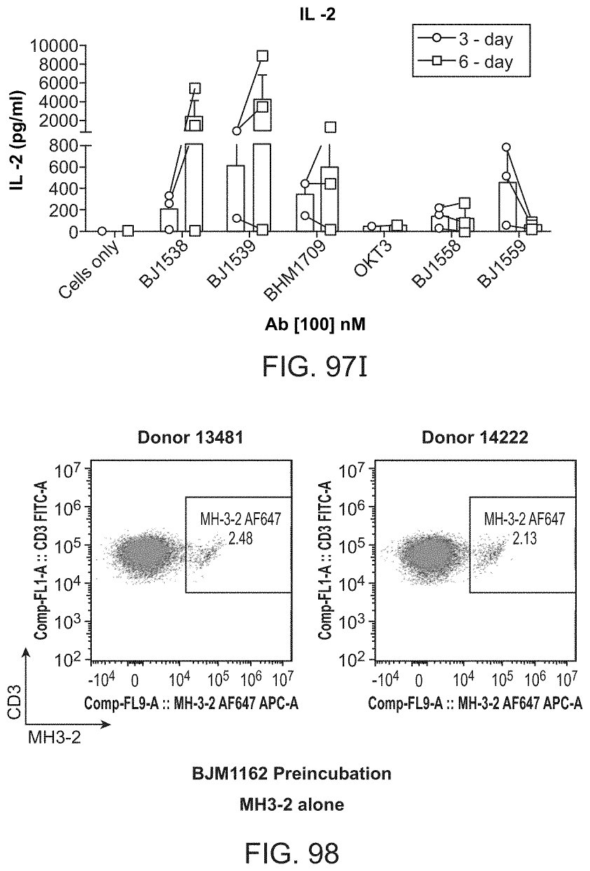

To assess whether two antibodies that bind TCRVβ 5-5, 5-6, TM23 and MH3-2, that do not share substantial sequence homology compete for binding to an overlapping epitope purified MH3-2 antibodies were conjugated to AF647; and T cells from two donors were preincubated or not with 500 nM TM23 and then stained with MH3-2 AF647. The data shows that preincubation with TM23 blocks MH3-2 binding (FIG. 98 and FIG. 99).

### Example 20. Polyfunctional Strength Index of Anti-TCRVβ 6-5 Antibody Expanded T Cells

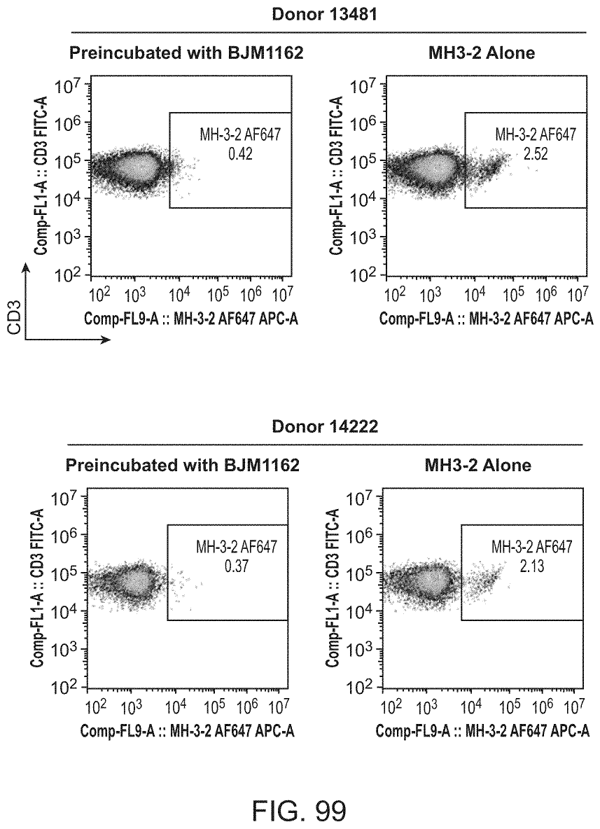

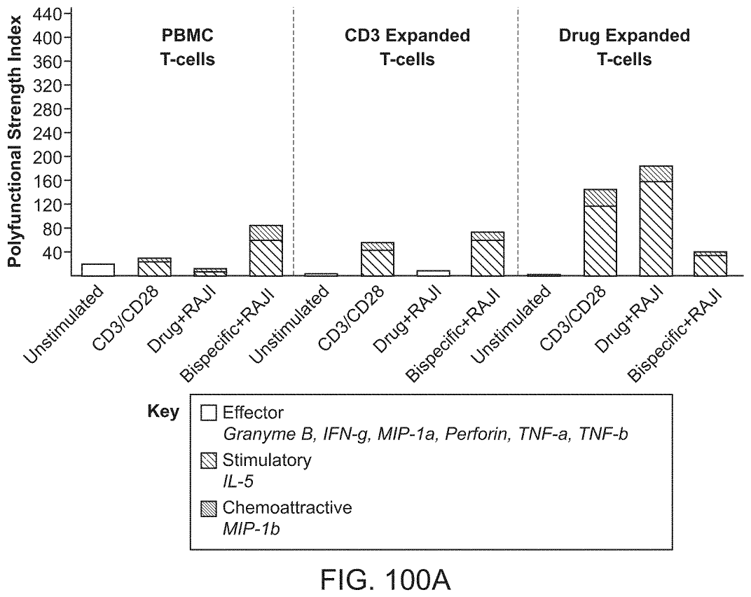

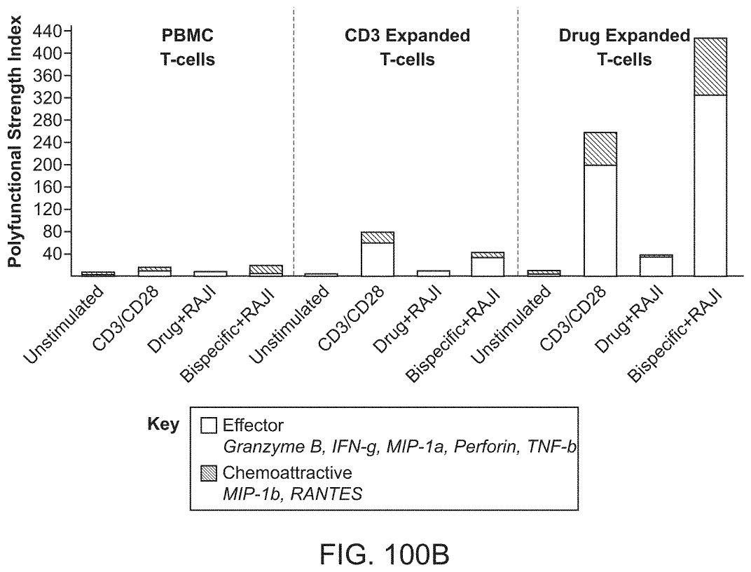

The polyfunctional strength index (PSI) of PBMCs were compared to anti-CD3ε antibody expanded CD4+ T cell (FIG. 100A) and CD8+ T cells (FIG. 100B) and anti-TCRVβ 6-5 antibody expanded (Drug Expanded T cells) CD4+ T cells (FIG. 100A) and CD8+ T cells (FIG. 100B). PSI is defined as the percentage of polyfunctional cells in the sample, multiplied by the intensities of the secreted cytokines. The data shows that there is a greater upregulation of PSI in the CD4+ T cells (FIG. 100A) and CD8+ T cells (FIG. 100B) across the groups expanded with anti-TCRVβ 6-5 antibody.

### Example 21: Ex Vivo Expansion of Anti-TCRVβ Antibody-Activated and Expanded CAR T Cells

At Day 0 plates were coated with antibodies and cryopreserved PBMCs (NK cell depleted) were thawed and added to the plate. 6 wells were coated with CD3/CD28 monoclonal antibodies at 0.1 μg/ml and 6 wells coated with anti-TCRVβ antibodies BHM1675 and BHM1709. The PBMCs were from one of three donors: 177, 178, and 890. The PBMCs were suspended at 1 or 2 million cells/ml in CAR-T cell medium with or without IL-2 (10 ng/ml). The 12 cultures are outlined in Table 12 below. At Day 1 virus was added with a transduction enhancer. 23 μl PMC152 virus (FLAG-tagged anti-CD19 CAR) was added to each well along with IL-2 (only in the wells that were not incubated with IL-2 on Day 0). At Day 2 PMC152 virus (FLAG-tagged anti-CD19 CAR) was added (17 μl). At Days 4-11 the cells were expanded in culture, and the number of cells counted on Days 7, 9, and 11. At Day 11 the cells were analyzed. The cells were analyzed by flow cytometry for expression for the CAR along with (1) CD45RA and CCR7 or (2) CD26L and CD45RO. The flow cytometry staining protocol is shown in FIG. 102. The cells were stained with PE-anti-FLAG A-AAD and wither CCR7-APC+CD45RA-FITC or CD26L-FITC and CD45RO-APC. Gating was done on live cells. The cells were further analyzed using a xCELLigence real-time cell analysis (RTCA) cytotoxicity assay. HeLa-CD19 target cells were used, and the assay medium was assayed for IFNγ and IL-2 levels by ELISA. The cells were also cryopreserved—6 vials of each culture with 5-6 million cells per vial.

TABLE 12
Twelve Culture Conditions

| Antibody | Cells | Virus | IL-2 at Day 0 |
|---|---|---|---|
| α-CD3/CD28 | 1 million/ml | No virus | +IL-2 |
| | PBMC #177 | | |
| α-CD3/CD28 | 1 million/ml | PMC152 | +IL-2 |
| | PBMC #177 | | |
| BHM1675/BHM1709 | 2 million/ml | PMC152 | +IL-2 |
| | PBMC #177 | | |
| BHM1675/BHM1709 | 2 million/ml | PMC152 | No IL-2 |
| | PBMC #177 | | |
| α-CD3/CD28 | 1 million/ml | No virus | +IL-2 |
| | PBMC #178 | | |
| α-CD3/CD28 | 1 million/ml | PMC152 | +IL-2 |
| | PBMC #178 | | |
| BHM1675/BHM1709 | 2 million/ml | PMC152 | +IL-2 |
| | PBMC #178 | | |
| BHM1675/BHM1709 | 2 million/ml | PMC152 | No IL-2 |
| | PBMC #178 | | |
| α-CD3/CD28 | 1 million/ml | No virus | +IL-2 |
| | PBMC #890 | | |
| α-CD3/CD28 | 1 million/ml | PMC152 | +IL-2 |
| | PBMC #890 | | |
| BHM1675/BHM1709 | 2 million/ml | PMC152 | +IL-2 |
| | PBMC #890 | | |
| BHM1675/BHM1709 | 2 million/ml | PMC152 | No IL-2 |
| | PBMC #890 | | |

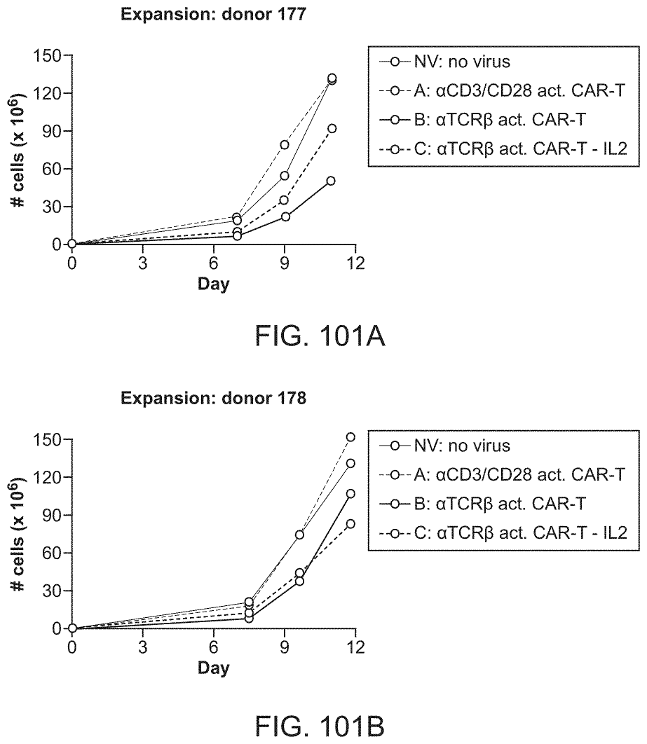

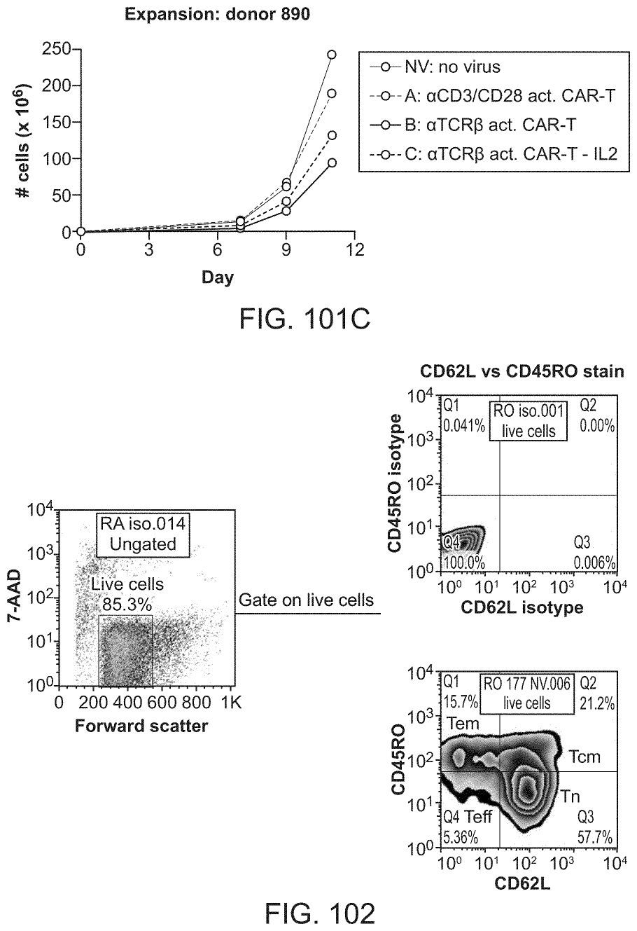

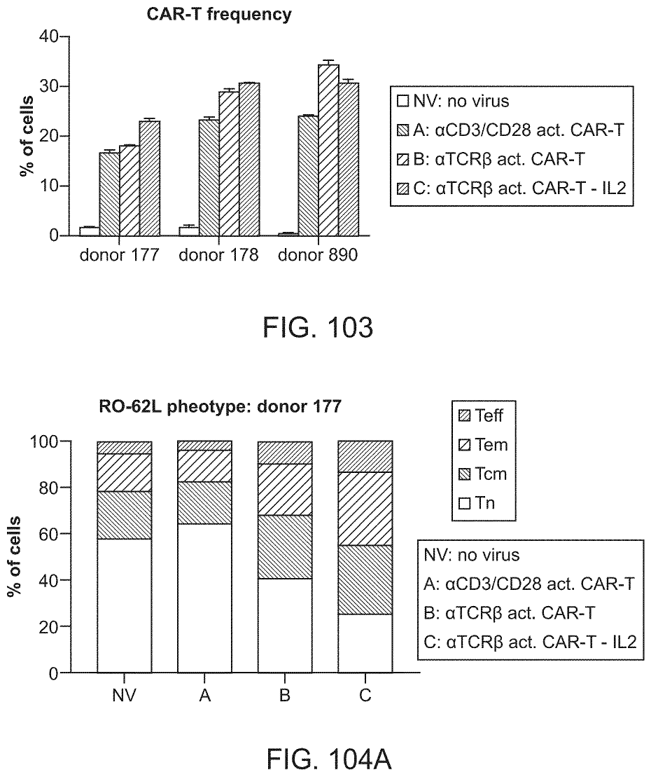

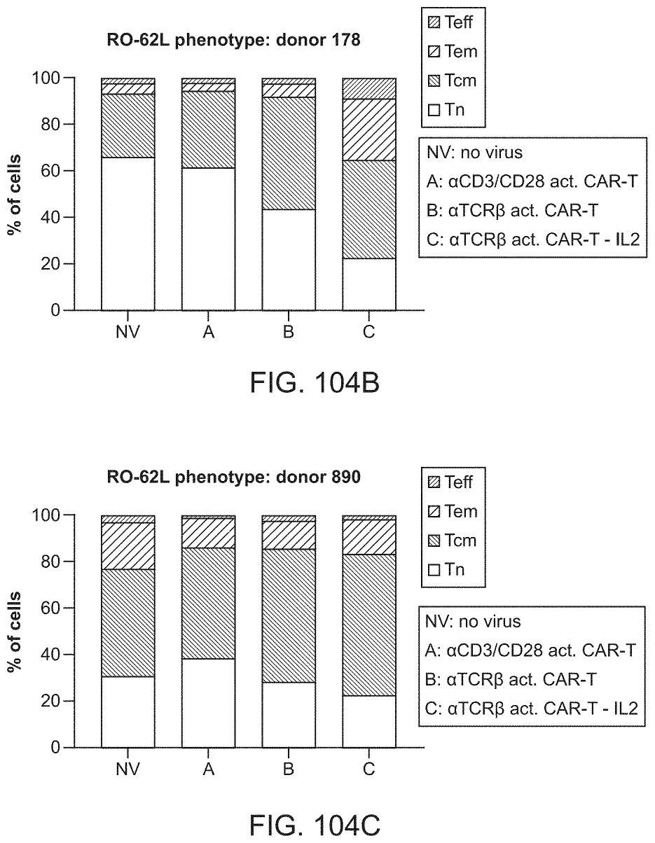

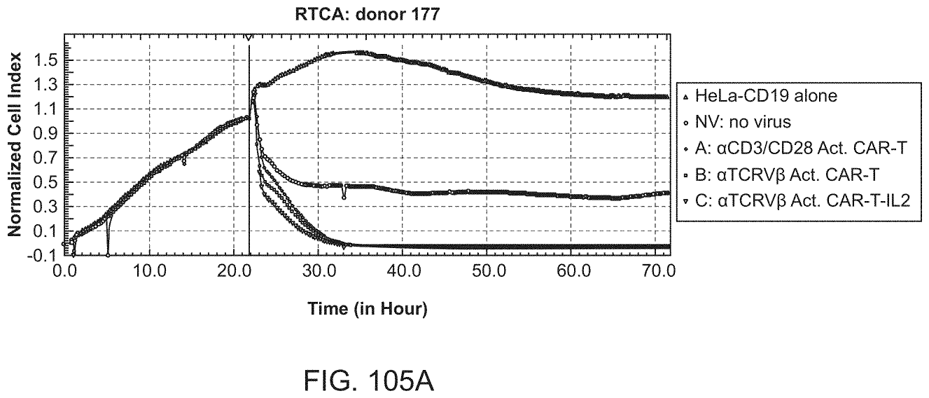

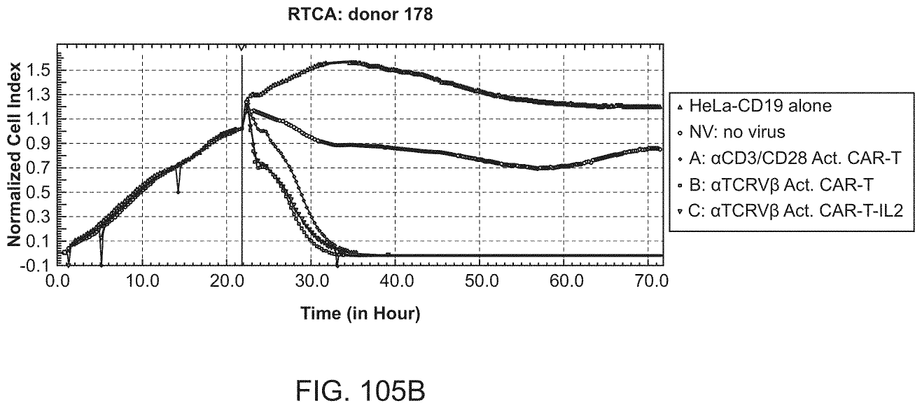

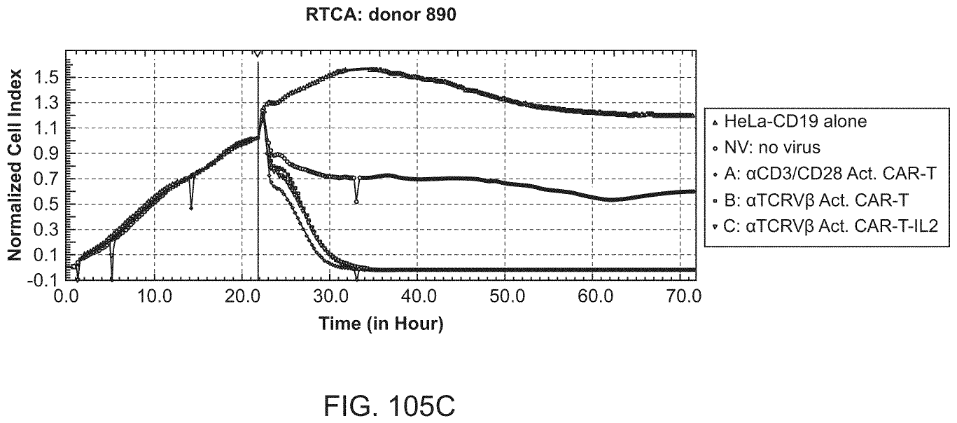

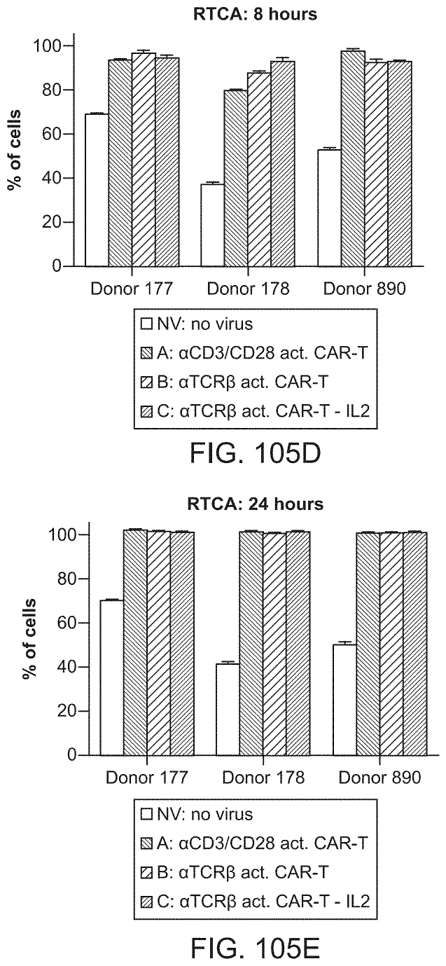

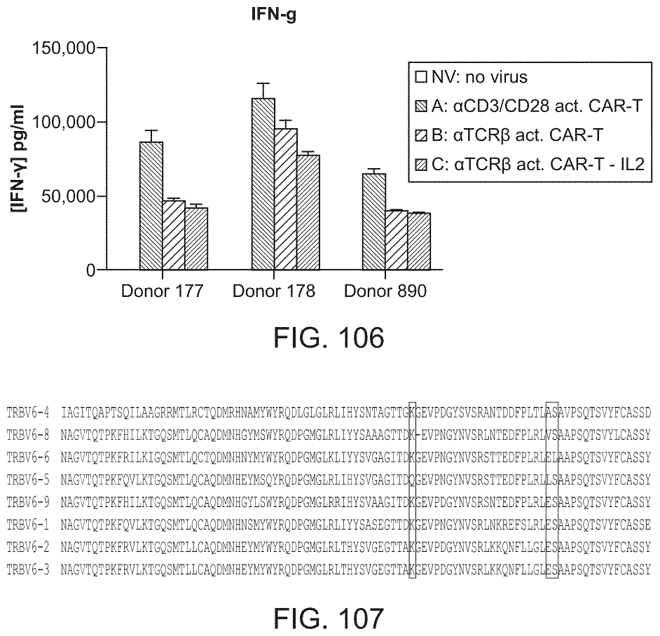

As shown in FIG. 101A-101C CAR-T cells expanded similarly (slightly lower) when activated with the anti-TCRVβ antibodies BHM1675 and BHM1709 as compared to CAR-T cells activated with the α-CD3/CD28 antibodies. The data further shows that IL-2 is not required for the first day when activating CAR-T cells with the anti-TCRVβ antibodies BHM1675 and BHM1709 (FIG. 101A-101C). FIG. 103 shows CAR-T cell frequencies are slightly higher when the cells are activated with the anti-TCRVβ antibodies BHM1675 and BHM1709 as compared to CAR-T cells activated with the a-CD3/CD28 antibodies (as determined by flow cytometry) and that IL-2 does not affect CAR-T frequency in a dose dependent manner. FIG. 104A-104C show CAR-T cells are more differentiated when the cells are activated with the anti-TCRVβ antibodies BHM1675 and BHM1709 as compared to CAR-T cells activated with the α-CD3/CD28 antibodies and IL-2 may increase CAR-T cell differentiation depending on the donor. FIG. 105A FIG. 105E shows that CAR-T cells activated with the anti-TCRVβ antibodies BHM1675 and BHM1709 exhibit comparable cytotoxicity to CAR-T cells activated with the α-CD3/CD28 antibodies; and IL-2 may increase CAR-T cell cytotoxicity depending on the donor. FIG. 106 shows that CAR-T cells activated with the anti-TCRVβ antibodies BHM1675 and BHM1709 produce less IFNγ compared to CAR-T cells activated with the α-CD3/CD28 antibodies.

### Example 22: Epitope Mapping of H131

Anti-hFc biosensors were equilibrated in PBS. Ligand: BJM0898-20191004 was diluted to 10 ug/mL in PBS. Analyte: BIM0444 or BJM1170 or BJM1171 or BJM1172 were diluted to 250 nM in PBS and then serially diluted two-fold down the plate. Assay was run according to the steps in Table 13.

TABLE 13
Assay Steps

| Step Time | Step Type Name |
|---|---|
| 30 | Baseline |
| to 0.5 nm | Loading |
| 30 | Baseline |
| 60 | Association |
| 300 | Dissociation |

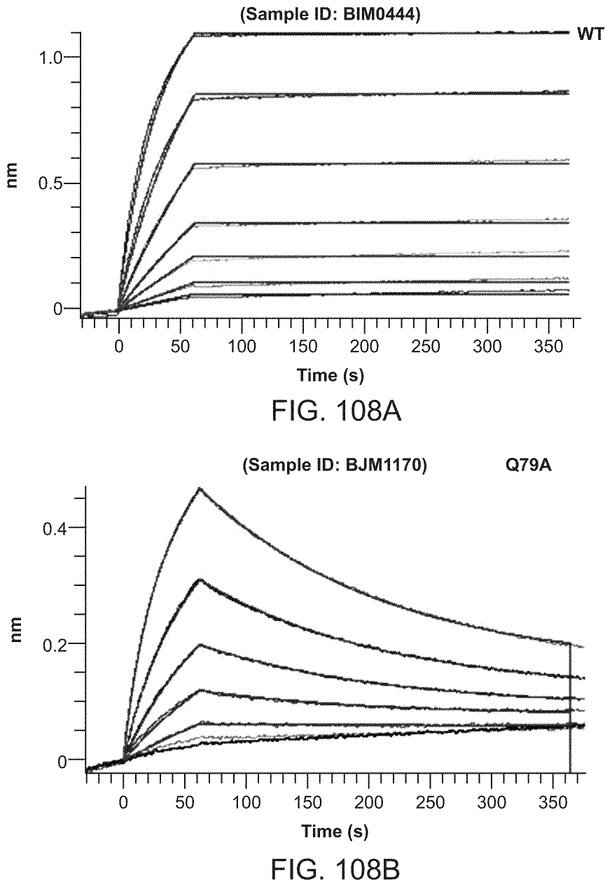

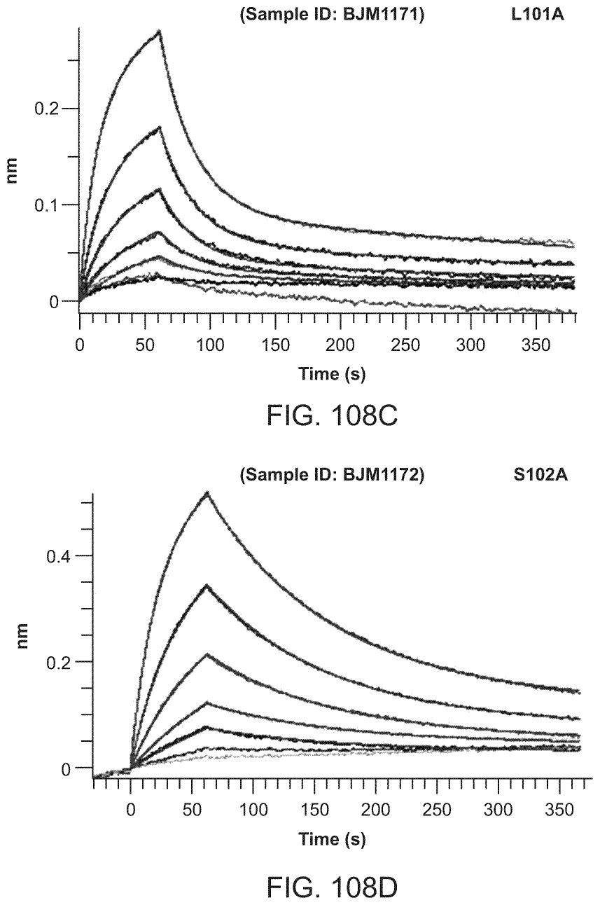

Sequence alignment of 8 functional human TCRVβ6 family sequences showed 3 unique amino acids in subfamily 6-5 (FIG. 107), positions Q79, L101, and S102 are unique to TCRVβ 6-5. Alanine substitutions at positions Q79, L101, and S102 significantly reduced binding of the antibody H131 to TCR compared to the WT receptor (FIG. 108A-108D).

We claim:

1-179. (canceled)

180. A method of expanding T cells that expresses a T cell receptor beta variable region (TCRβV) in a T cell population ex vivo, the method comprising:
contacting the T cell population with a first agent comprising a first domain that binds to a first target molecule that is T cell receptor beta variable beta chain (TCRβV),
wherein the first agent comprises a second domain that binds to a second target molecule that is a protein expressed on the surface of T cells of the T cell population,
wherein the first target molecule and the second target molecule are different target molecules,
wherein the first domain contacts the TCRβV of a T cell receptor (TCR) expressed by the T cells in the T cell population, thereby expanding the T cells in the T cell population, and
wherein the T cell population is an ex vivo T cell population.

181. The method of claim 180, wherein T cells in the T cell population express a chimeric antigen receptor (CAR) or a recombinant T cell receptor.

182. The method of claim 180, wherein the method expands T cells expressing the CAR or the recombinant T cell receptor.

183. The method of claim 182, wherein the first domain binds to a TCRβV region of a TCRβV belonging to a TCRβV12 subfamily, a TCRβV6 subfamily, a TCRβV20 subfamily or a TCRβV5 subfamily.

184. The method of claim 183, wherein the second domain binds to a T cell receptor variable beta chain (TCRβV).

185. The method of claim 182, wherein the second domain and the first domain bind to TCRβVs belonging to different subfamilies or different members of the same TCRβV subfamily.

186. The method of claim 185, wherein the method the method comprises culturing the T cell population in the presence of the first agent for at least 5 days.

187. The method of claim 180, wherein T cells in an expanded T cell population obtained by the method exhibit a more activated signature based on a principal component analysis of CD27, CD28, CD96, CD40LG, ICOS, TNFRSF9 (4-1BB), CD276, CSF2 (GM-CSF), CD80, CD86, CCL3, CCL4, CXCR3, CXCL9, CXCL10, or a combination thereof compared to an expanded T cell population obtained by a method that expands the T cell population with an anti-CD3 agent.

188. The method of claim 187, wherein T cells in an expanded T cell population obtained by the method exhibit a less exhausted signature based on a principal component analysis of PD-1 (PDCD1), LAG3, Tim-3 (HAVCR2), CTLA4, BTLA, CD244 (2B4), CD160, CD39 (ENTPD1), TIGIT, or a combination thereof compared to an expanded T cell population obtained by a method that expands the T cell population with an anti-CD3 agent.

189. The method of claim 180, wherein the TCRβV is TCRβV2, TCRβV3, TCRβV4, TCRβV5, TCRβV6, TCRβV7, TCRβV8, TCRβV9, TCRβV10, TCRβV11, TCRβV12, TCRβV19, TCRβV20, TCRβV24, TCRβV25, TCRβV27, TCRβV28, TCRβV29, or TCRβV30.

190. The method of claim 180, wherein the TCRβV is TCRβV2, TCRβ4-1, TCRβV4-2, TCRβV5-1, TCRβV5-5, TCRβV5-6, TCRβV6, TCRβ6-5, TCRβV6-6, TCRβV6-9, TCRβV7-2, TCRβV7-3, TCRβV7-8, TCRβV7-9, TCRβV9, TCRβV10-1, TCRβV10-2, TCRβV10-3, TCRβV11-2, TCRβV12-3, TCRβV12-4, TCRβV12-5, TCRβV19, TCRβV20-1, TCRβV24-1, TCRβV25-1, or TCRβV28.

191. The method of claim 180, wherein the TCRβV is TCRβV2, TCRβV3-1, TCRβV4-1, TCRβ4-2, TCRβV5-1, TCRβV5-4, TCRβV5-5, TCRβV5-6, TCRβV6-1, TCRβV6-5, TCRβV6-6, TCRβV7-3, TCRβV7-6, TCRβV7-8, TCRβV9, TCRβV11-2, TCRβV19, TCRβV20-1, TCRβV24-1, TCRβV27, TCRβV28, TCRβV29-1, or TCRβV30.

192. The method of claim 180, wherein second target molecule is selected from the group consisting of BCMA, FcRH5, CD19, CD20, CD22, CD30, CD33, CD38, CD47, CD99, CD123, FcRH5, CLEC12, CD179A, SLAMF7, NY-ESO1, PDL1, CD47, gangloside 2 (GD2), prostate stem cell antigen (PSCA), prostate specific membrane antigen (PSMA), prostate-specific antigen (PSA), carcinoembryonic antigen (CEA), Ron Kinase, c-Met, immature laminin receptor, TAG-72, BING-4, Calcium-activated chloride channel 2, Cyclin-Bl, 9D7, Ep-CAM, EphA3, Her2/neu, Telomerase, SAP-I, Survivin, NY-ESO-1/LAGE-1, PRAME, SSX-2, Melan-A/MART-I, Gp100/pmell7, Tyrosinase, TRP-1/-2, MC1R, b-catenin, BRCA1/2, CDK4, CML66, Fibronectin, p53, Ras, TGF-B receptor, AFP, ETA, MAGE, MUC-1, CA-125, BAGS, GAGE, NY-ESO-l, b-catenin, CDK4, CDC27, a actinin-4, TRP1/gp75, TRP2, gp100, Melan-A/MART1, gangliosides, WT1, EphA3, Epidermal growth factor receptor (EGFR), MART-2, MART-1, MUC1, MUC2, MUM1, MUM2, MUM3, NA88-1, NPM, OA1, OGT, RCC, RUI1, RUI2, SAGE, TRG, TRP1, TSTA, Folate receptor alpha, L1-CAM, CAIX, gpA33, GD3, GM2, VEGFR, Intergrin, a carbohydrate, IGF1R, EPHA3, TRAILR1, TRAILR2, RANKL, FAP, TGF-beta, hyaluronic acid, collagen, tenascin C, and tenascin W.

193. The method of claim 180, wherein the method further comprises contacting the T cell population with a second agent, wherein the second agent comprises a domain that binds to a TCRβV, wherein the first agent and the second agent bind to TCRβVs that belong to different TCRβV subfamilies or that are different members of the same TCRβV subfamily.

194. The method of claim 193, wherein the T cell population is from a biological sample from a human subject.

195. The method of claim 193, wherein the second target molecule s not a TCRβV.

196. The method of claim 193, wherein the second target molecule is CD19.

197. The method of claim 193, wherein the second target molecule is CD3.

198. The method of claim 193, wherein the second target molecule is a target molecule on a target cell within the T cell population.

199. The method of claim 181, wherein the first domain binds to a TCRβV region of a TCRβV12-3, a TCRβV12-4, or a combination thereof.

200. The method of claim 199, wherein the first domain binds to a TCRβV region of a TCRβV6-5.

201. The method of claim 18283, wherein the first domain binds to a TCRβV region of a TCRβV20-1.

202. The method of claim 183, wherein the first domain binds to a TCRβV region of a TCRβV5-1.

203. The method of claim 180, wherein the first agent is coupled to a solid surface.

204. The method of claim 203, wherein the solid surface is a bead or a cell culture plate.

205. The method of claim 180, wherein binding of the first domain to the TCRβV and binding of the second domain to the second target molecule promotes the T cells to kill cancer cells.

206. The method of claim 180, wherein the target cell is a T cell.

207. The method of claim 180, wherein the target cell is a non-cancer cell.


SEQUENCE LISTING (386 sequences)

SEQ ID NO: 1 - protein, 10 aa, Artificial Sequence
  Description of Artificial Sequence Synthetic peptide
GYSFTTYYIH

SEQ ID NO: 2 - protein, 17 aa, Artificial Sequence
  Description of Artificial Sequence Synthetic peptide
WFFPGSGNIKYNEKFKG

SEQ ID NO: 3 - protein, 10 aa, Artificial Sequence
  Description of Artificial Sequence Synthetic peptide
SYYSYDVLDY

SEQ ID NO: 4 - protein, 5 aa, Artificial Sequence
  Description of Artificial Sequence Synthetic peptide
TYYIH

SEQ ID NO: 5 - protein, 7 aa, Artificial Sequence
  Description of Artificial Sequence Synthetic peptide
GYSFTTY

SEQ ID NO: 6 - protein, 6 aa, Artificial Sequence
  Description of Artificial Sequence Synthetic peptide
FPGSGN

SEQ ID NO: 7 - protein, 11 aa, Artificial Sequence
  Description of Artificial Sequence Synthetic peptide
KASQNVGINVV

SEQ ID NO: 8 - protein, 7 aa, Artificial Sequence
  Description of Artificial Sequence Synthetic peptide
SSSHRYS

SEQ ID NO: 9 - protein, 9 aa, Artificial Sequence
  Description of Artificial Sequence Synthetic peptide
QQFKSYPLT

SEQ ID NO: 10 - protein, 107 aa, Artificial Sequence
  Description of Artificial Sequence Synthetic polypeptide
DILMTQSQKFMSTSLGDRVSVSCKASQNVGINVVWHQQKPGQSPKALIYSSSHRYSGVPDRFTGSGSGTDFTLTINNVQSEDLAEYFCQQFKSYPLTFGAGTKLELK

SEQ ID NO: 11 - protein, 119 aa, Artificial Sequence
  Description of Artificial Sequence Synthetic polypeptide
QVQLQQSGPELVKPGTSVKISCKASGYSFTTYYIHWVKQRPGQGLEWIGWFFPGSGNIKYNEKFKGKATLTADTSSSTAYMQLSSLTSEESAVYFCAGSYYSYDVLDYWGHGTTLTVSS

SEQ ID NO: 12 - protein, 119 aa, Artificial Sequence
  Description of Artificial Sequence Synthetic polypeptide
QVQLVQSGAEVKKPGSSVKVSCKASGYSFTTYYIHWVRQAPGQGLEWMGWFFPGSGNIKYNEKFKGRVTITADTSTSTAYMELSSLRSEDTAVYYCAGSYYSYDVLDYWGQGTTVTVSS

SEQ ID NO: 13 - DNA, 357 bp, Artificial Sequence
  Description of Artificial Sequence Synthetic polynucleotide
caggtgcagctggttcagtctggcgccgaagtgaagaaacctggctcctccgtgaaggtgtcctgcaaggcttccggctactccttcaccacctactacatccactgggtccgacaggcccctggacaaggattggaatggatgggctggttcttccccggctccggcaacatcaagtacaacgagaagttcaagggccgcgtgaccatcaccgccgacacctctacctctaccgcctacatggaactgtccagcctgagatctgaggacaccgccgtgtactactgcgccggctcctactactcttacgacgtgctggattactggggccagggcaccacagtgacagtgtcctct

SEQ ID NO: 14 - protein, 107 aa, Artificial Sequence
  Description of Artificial Sequence Synthetic polypeptide
DIQMTQSPSFLSASVGDRVTITCKASQNVGINVVWHQQKPGKAPKALIYSSSHRYSGVPSRFSGSGSGTEFTLTISSLQPEDFATYFCQQFKSYPLTFGQGTKLEIK

SEQ ID NO: 15 - DNA, 321 bp, Artificial Sequence
  Description of Artificial Sequence Synthetic polynucleotide
gacatccagatgacccagtctccatccttcctgtccgcctctgtgggcgacagagtgaccatcacatgcaaggcctctcagaacgtgggcatcaacgtcgtgtggcaccagcagaagcctggcaaggctcctaaggctctgatctactcctccagccaccggtactctggcgtgccctctagattttccggctctggctctggcaccgagtttaccctgacaatctccagcctgcagcctgaggacttcgccacctacttttgccagcagttcaagagctaccctctgacctttggccagggcaccaagctggaaatcaag

SEQ ID NO: 16 - protein, 107 aa, Artificial Sequence
  Description of Artificial Sequence Synthetic polypeptide
DIQMTQSPSSLSASVGDRVTITCKASQNVGINVVWHQQKPGKVPKALIYSSSHRYSGVPSRFSGSGSGTDFTLTISSLQPEDVATYFCQQFKSYPLTFGQGTKLEIK

SEQ ID NO: 17 - DNA, 321 bp, Artificial Sequence
  Description of Artificial Sequence Synthetic polynucleotide
gacatccagatgacccagtctccatcctctctgtccgcctctgtgggcgacagagtgaccatcacatgcaaggcctctcagaacgtgggcatcaacgtcgtgtggcaccagcagaaacctggcaaggtgcccaaggctctgatctactcctccagccacagatactccggcgtgccctctagattctccggctctggctctggcaccgactttaccctgacaatctccagcctgcagcctgaggacgtggccacctacttttgccagcagttcaagagctaccctctgacctttggccagggcaccaagctggaaatcaag

SEQ ID NO: 18 - protein, 106 aa, Artificial Sequence
  Description of Artificial Sequence Synthetic polypeptide
QSVLTQPPSVSEAPRQRVTISCKASQNVGINVVWHQQLPGKAPKALIYSSSHRYSGVSDRFSGSGSGTSFSLAISGLQSEDEADYFCQQFKSYPLTFGTGTKVTVL

SEQ ID NO: 19 - protein, 119 aa, Artificial Sequence
  Description of Artificial Sequence Synthetic polypeptide
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSS

SEQ ID NO: 20 - protein, 119 aa, Artificial Sequence
  Description of Artificial Sequence Synthetic polypeptide
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAVSYYSYDVLDYWGQGTTVTVSS

SEQ ID NO: 21 - protein, 119 aa, Artificial Sequence
  Description of Artificial Sequence Synthetic polypeptide
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAVSYYSYDVLDYWGQGTTVTVSS

SEQ ID NO: 22 - protein, 107 aa, Artificial Sequence
  Description of Artificial Sequence Synthetic polypeptide
DIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 23 - protein, 107 aa, Artificial Sequence
  Description of Artificial Sequence Synthetic polypeptide
DIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 24 - protein, 246 aa, Artificial Sequence
  Description of Artificial Sequence Synthetic polypeptide
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 25 - protein, 246 aa, Artificial Sequence
  Description of Artificial Sequence Synthetic polypeptide
QVQLVQSGAEVKKPGSSVKVSCKASGHDFRDFYIHWVRQAPGQGLEWMGRVYPGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 26 - protein, 246 aa, Artificial Sequence
  Description of Artificial Sequence Synthetic polypeptide
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 27 - protein, 246 aa, Artificial Sequence
  Description of Artificial Sequence Synthetic polypeptide
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVENKVAWHQQKPGKAPKALIYSSSHRYKGVPSRFSGSGSGTEFTLTISSLQPEDFATYFCQQFKSYPLTFGQGTKLEIK

SEQ ID NO: 28 - protein, 246 aa, Artificial Sequence
  Description of Artificial Sequence Synthetic polypeptide
QVQLVQSGAEVKKPGSSVKVSCKASGHDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 29 - protein, 246 aa, Artificial Sequence
  Description of Artificial Sequence Synthetic polypeptide
QVQLVQSGAEVKKPGSSVKVSCKASGHDFDKFYIHWVRQAPGQGLEWMGRIFAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 30 - protein, 246 aa, Artificial Sequence
  Description of Artificial Sequence Synthetic polypeptide
QVQLVQSGAEVKKPGSSVKVSCKASGTDFKLTYIHWVRQAPGQGLEWMGRVSPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 31 - protein, 246 aa, Artificial Sequence
  Description of Artificial Sequence Synthetic polypeptide
QVQLVQSGAEVKKPGSSVKVSCKASGTDFKLTYIHWVRQAPGQGLEWMGRIFPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWYQQKPGKAPKALIYSSSHRYKGVPSRFSGSGSGTEFTLTISSLQPEDFATYFCQQFKSYPLTFGQGTKLEIK

SEQ ID NO: 32 - protein, 246 aa, Artificial Sequence
  Description of Artificial Sequence Synthetic polypeptide
QVQLVQSGAEVKKPGSSVKVSCKASGGTFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 33 - protein, 246 aa, Artificial Sequence
  Description of Artificial Sequence Synthetic polypeptide
QVQLVQSGAEVKKPGSSVKVSCKASGTDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 34 - protein, 246 aa, Artificial Sequence; identical to SEQ ID NO: 33
  Description of Artificial Sequence Synthetic polypeptide

SEQ ID NO: 35 - protein, 246 aa, Artificial Sequence
  Description of Artificial Sequence Synthetic polypeptide
QVQLVQSGAEVKKPGSSVKVSCKASGHDFRLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 36 - protein, 246 aa, Artificial Sequence
  Description of Artificial Sequence Synthetic polypeptide
QVQLVQSGAEVKKPGSSVKVSCKASGHDFHLWYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNKVAWHQQKPGKAPKALIYSSSHRYKGVPSRFSGSGSGTEFTLTISSLQPEDFATYFCQQFKSYPLTFGQGTKLEIK

SEQ ID NO: 37 - protein, 246 aa, Artificial Sequence
  Description of Artificial Sequence Synthetic polypeptide
QVQLVQSGAEVKKPGSSVKVSCKASGTDFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGNRVAWYQQKPGKAPKALIYSSSHRYKGVPSRFSGSGSGTEFTLTISSLQPEDFATYFCQQFKSYPLTFGQGTKLEIK

SEQ ID NO: 38 - protein, 246 aa, Artificial Sequence
  Description of Artificial Sequence Synthetic polypeptide
QVQLVQSGAEVKKPGSSVKVSCKASGHDFHLWYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWHQQKPGKAPKALIYSSSHRYKGVPSRFSGSGSGTEFTLTISSLQPEDFATYFCQQFKSYPLTFGQGTKLEIK

SEQ ID NO: 39 - protein, 246 aa, Artificial Sequence
  Description of Artificial Sequence Synthetic polypeptide
QVQLVQSGAEVKKPGSSVKVSCKASGHDFKLTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 40 - protein, 246 aa, Artificial Sequence
  Description of Artificial Sequence Synthetic polypeptide
QVQLVQSGAEVKKPGSSVKVSCKASGTDFHLWYIHWVRQAPGQGLEWMGRVFAGSGSYRYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 41 - protein, 246 aa, Artificial Sequence
  Description of Artificial Sequence Synthetic polypeptide
QVQLVQSGAEVKKPGSSVKVSCKASGHDFDKTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVEDRVAWYQQKPGKAPKALIYSSSHRYKGVPSRFSGSGSGTEFTLTISSLQPEDFATYFCQQFKSYPLTFGQGTKLEIK

SEQ ID NO: 42 - protein, 246 aa, Artificial Sequence
  Description of Artificial Sequence Synthetic polypeptide
QVQLVQSGAEVKKPGSSVKVSCKASGHDFDKTYIHWVRQAPGQGLEWMGRIYPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVADRVAWYQQKPGKAPKALIYSSSHRYKGVPSRFSGSGSGTEFTLTISSLQPEDFATYFCQQFKSYPLTFGQGTKLEIK

SEQ ID NO: 43 - protein, 246 aa, Artificial Sequence
  Description of Artificial Sequence Synthetic polypeptide
QVQLVQSGAEVKKPGSSVKVSCKASGTDFDKTYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 44 - protein, 246 aa, Artificial Sequence
  Description of Artificial Sequence Synthetic polypeptide
QVQLVQSGAEVKKPGSSVKVSCKASGTDFDKIYIHWVRQAPGQGLEWMGRISAGSGNIKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 45 - protein, 246 aa, Artificial Sequence
  Description of Artificial Sequence Synthetic polypeptide
QVQLVQSGAEVKKPGSSVKVSCKASGTDFDKIYIHWVRQAPGQGLEWMGRISAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVAWYQQKPGKAPKALIYSSSHRYKGVPSRFSGSGSGTEFTLTISSLQPEDFATYFCQQFKSYPLTFGQGTKLEIK

SEQ ID NO: 46 - protein, 246 aa, Artificial Sequence
  Description of Artificial Sequence Synthetic polypeptide
QVQLVQSGAEVKKPGSSVKVSCKASGGTFKLTYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDDRVAWYQQKPGKAPKALIYSSSHRYKGVPSRFSGSGSGTEFTLTISSLQPEDFATYFCQQFKSYPLTFGQGTKLEIK

SEQ ID NO: 47 - protein, 246 aa, Artificial Sequence
  Description of Artificial Sequence Synthetic polypeptide
QVQLVQSGAEVKKPGSSVKVSCKASGTDFKLTYIHWVRQAPGQGLEWMGRISPGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVDNRVAWHQQKPGKAPKALIYSSSHRYKGVPSRFSGSGSGTEFTLTISSLQPEDFATYFCQQFKSYPLTFGQGTKLEIK

SEQ ID NO: 48 - protein, 246 aa, Artificial Sequence
  Description of Artificial Sequence Synthetic polypeptide
QVQLVQSGAEVKKPGSSVKVSCKASGTDFDKFYIHWVRQAPGQGLEWMGRVSAGSGNVKYNEKFKGRVTITADTSTSTAYMELSSLRSEDTAVYYCAGSYYSYDVLDYWGQGTTVTVSSGGGGSGGGGSGGGGSGGGGSDIQMTQSPSFLSASVGDRVTITCKASQNVGDRVVWYQQKPGKAPKALIYSSSHRYKGVPSRFSGSGSGTEFTLTISSLQPEDFATYFCQQFKSYPLTFGQGTKLEIK

SEQ ID NO: 49 - protein, 10 aa, Artificial Sequence
  Description of Artificial Sequence Synthetic peptide
GFTFSNFGMH

SEQ ID NO: 50 - protein, 17 aa, Artificial Sequence
  Description of Artificial Sequence Synthetic peptide
YISSGSSTIYYADTLKG

SEQ ID NO: 51 - protein, 8 aa, Artificial Sequence
  Description of Artificial Sequence Synthetic peptide
RGEGAMDY

SEQ ID NO: 52 - protein, 5 aa, Artificial Sequence
  Description of Artificial Sequence Synthetic peptide
NFGMH

SEQ ID NO: 53 - protein, 7 aa, Artificial Sequence
  Description of Artificial Sequence Synthetic peptide
GFTFSNF

SEQ ID NO: 54 - protein, 6 aa, Artificial Sequence
  Description of Artificial Sequence Synthetic peptide
SSGSST

SEQ ID NO: 55 - protein, 10 aa, Artificial Sequence
  Description of Artificial Sequence Synthetic peptide
RASSSVNYIY

SEQ ID NO: 56 - protein, 7 aa, Artificial Sequence
  Description of Artificial Sequence Synthetic peptide
YTSNLAP

SEQ ID NO: 57 - protein, 9 aa, Artificial Sequence
  Description of Artificial Sequence Synthetic peptide
QQFTSSPFT

SEQ ID NO: 58 - protein, 117 aa, Artificial Sequence
  Description of Artificial Sequence Synthetic polypeptide
DVQLVESGGGLVQPGGSRKLSCAASGFTFSNFGMHWVRQAPDKGLEWVAYISSGSSTIYYADTLKGRFTISRDNPKNTLFLQMTSLRSEDTAMYYCARRGEGAMDYWGQGTSVTVSS

SEQ ID NO: 59 - protein, 106 aa, Artificial Sequence
  Description of Artificial Sequence Synthetic polypeptide
ENVLTQSPAIMSASLGEKVTMSCRASSSVNYIYWYQQKSDASPKLWIYYTSNLAPGVPTRFSGSGSGNSYSLTISSMEGEDAATYYCQQFTSSPFTFGSGTKLEIK

SEQ ID NO: 60 - protein, 106 aa, Artificial Sequence
  Description of Artificial Sequence Synthetic polypeptide
DIQMTQSPSSLSASVGDRVTITCRASSSVNYIYWYQQTPGKAPKLLIYYTSNLAPGVPSRFSGSGSGTDYTFTISSLQPEDIATYYCQQFTSSPFTFGQGTKLQIT

SEQ ID NO: 61 - protein, 117 aa, Artificial Sequence
  Description of Artificial Sequence Synthetic polypeptide
EVQLVESGGGVVQPGRSLRLSCSSSGFTFSNFGMHWVRQAPGKGLEWVAYISSGSSTIYYADTLKGRFTISRDNSKNTLFLQMDSLRPEDTGVYFCARRGEGAMDYWGQGTPVTVSS

SEQ ID NO: 62 - protein, 106 aa, Artificial Sequence
  Description of Artificial Sequence Synthetic polypeptide
DNQLTQSPSFLSASVGDRVTITCRASSSVNYIYWYQQKPGKAPKLLIYYTSNLAPGVPSRFSGSGSGNEYTLTISSLQPEDFATYYCQQFTSSPFTFGQGTKLEIK

SEQ ID NO: 63 - protein, 117 aa, Artificial Sequence
  Description of Artificial Sequence Synthetic polypeptide
QVQLVESGGGVVQPGRSLRLSCAASGFTFSNFGMHWVRQAPGKGLEWVAYISSGSSTIYYADTLKGRFTISRDNSKNTLYLQMNSLRAEDTAVYYCARRGEGAMDYWGQGTTVTVSS

SEQ ID NO: 64 - protein, 117 aa, Artificial Sequence
  Description of Artificial Sequence Synthetic polypeptide
EVQLVESGGGLVQPGGSLRLSCAASGFTFSNFGMHWVRQAPGKGLEWVSYISSGSSTIYYADTLKGRFTISRDNSKNTLYLQMNSLRAEDTAVYYCARRGEGAMDYWGQGTTVTVSS

SEQ ID NO: 65 - protein, 106 aa, Artificial Sequence
  Description of Artificial Sequence Synthetic polypeptide
DNQLTQSPSSLSASVGDRVTITCRASSSVNYIYWYQQKPGKAPKLLIYYTSNLAPGVPSRFSGSGSGNDYTLTISSLQPEDFATYYCQQFTSSPFTFGQGTKLEIK

SEQ ID NO: 66 - protein, 117 aa, Artificial Sequence
  Description of Artificial Sequence Synthetic polypeptide
EVQLVESGGGLVQPGGSLRLSCAASGFTFSNFGMHWVRQAPGKGLEWVSYISSGSSTIYYADTLKGRFTISRDNAKNSLYLQMNSLRAEDTAVYYCARRGEGAMDYWGQGTTVTVSS

SEQ ID NO: 67 - protein, 106 aa, Artificial Sequence
  Description of Artificial Sequence Synthetic polypeptide
ENVLTQSPATLSVSPGERATLSCRASSSVNYIYWYQQKPGQAPRLLIYYTSNLAPGIPARFSGSGSGNEYTLTISSLQSEDFAVYYCQQFTSSPFTFGQGTKLEIK

SEQ ID NO: 68 - protein, 105 aa, Artificial Sequence
  Description of Artificial Sequence Synthetic polypeptide
QNVLTQPPSASGTPGQRVTISCRASSSVNYIYWYQQLPGTAPKLLIYYTSNLAPGVPDRFSGSGSGNSYSLAISGLRSEDEADYYCQQFTSSPFTFGTGTKVTVL

SEQ ID NO: 69 - protein, 105 aa, Artificial Sequence
  Description of Artificial Sequence Synthetic polypeptide
SNELTQPPSVSVSPGQTARITCRASSSVNYIYWYQQKSGQAPVLVIYYTSNLAPGIPERFSGSGSGNMYTLTISGAQVEDEADYYCQQFTSSPFTFGTGTKVTVL

SEQ ID NO: 70 - protein, 5 aa, Artificial Sequence
  Description of Artificial Sequence Synthetic peptide
AYGVN

SEQ ID NO: 71 - protein, 16 aa, Artificial Sequence
  Description of Artificial Sequence Synthetic peptide
MIWGDGNTDYNSALKS

SEQ ID NO: 72 - protein, 12 aa, Artificial Sequence
  Description of Artificial Sequence Synthetic peptide
DRVTATLYAMDY

SEQ ID NO: 73 - protein, 7 aa, Artificial Sequence
  Description of Artificial Sequence Synthetic peptide
GFSLTAY

SEQ ID NO: 74 - protein, 5 aa, Artificial Sequence
  Description of Artificial Sequence Synthetic peptide
WGDGN

SEQ ID NO: 75 - protein, 10 aa, Artificial Sequence
  Description of Artificial Sequence Synthetic peptide
GFSLTAYGVN

SEQ ID NO: 76 - protein, 11 aa, Artificial Sequence
  Description of Artificial Sequence Synthetic peptide
SASQGISNYLN

SEQ ID NO: 77 - protein, 7 aa, Artificial Sequence
  Description of Artificial Sequence Synthetic peptide
YTSSLHS

SEQ ID NO: 78 - protein, 9 aa, Artificial Sequence
  Description of Artificial Sequence Synthetic peptide
QQYSKLPRT

SEQ ID NO: 79 - protein, 107 aa, Artificial Sequence
  Description of Artificial Sequence Synthetic polypeptide
DIQMTQTTSSLSASLGDRVTISCSASQGISNYLNWYQQKPDGTVKLLIYYTSSLHSGVPSRFSGSGSGTDYSLTISNLEPEDIATYYCQQYSKLPRTFGGGTKVEIK

SEQ ID NO: 80 - protein, 120 aa, Artificial Sequence
  Description of Artificial Sequence Synthetic polypeptide
QVQLKESGPGLVAPSQSLSITCTVSGFSLTAYGVNWVRQPPGKGLEWLGMIWGDGNTDYNSALKSRLSISKDNSKSQVFLKMNSLQTDDTARYYCARDRVTATLYAMDYWGQGTSVTVSS

SEQ ID NO: 81 - protein, 107 aa, Artificial Sequence
  Description of Artificial Sequence Synthetic polypeptide
DIQMTQSPSSLSASVGDRVTITCSASQGISNYLNWYQQTPGKAPKLLIYYTSSLHSGVPSRFSGSGSGTDYTFTISSLQPEDIATYYCQQYSKLPRTFGQGTKLQIT

SEQ ID NO: 82 - protein, 120 aa, Artificial Sequence
  Description of Artificial Sequence Synthetic polypeptide
QVQLQESGPGLVRPSQTLSLTCTVSGFSLTAYGVNWVRQPPGRGLEWLGMIWGDGNTDYNSALKSRVTMLKDTSKNQFSLRLSSVTAADTAVYYCARDRVTATLYAMDYWGQGSLVTVSS

SEQ ID NO: 83 - protein, 120 aa, Artificial Sequence
  Description of Artificial Sequence Synthetic polypeptide
QVTLKESGPVLVKPTETLTLTCTVSGFSLTAYGVNWVRQPPGKALEWLGMIWGDGNTDYNSALKSRLTISKDNSKSQVVLTMTNMDPVDTATYYCARDRVTATLYAMDYWGQGTLVTVSS

SEQ ID NO: 84 - protein, 120 aa, Artificial Sequence
  Description of Artificial Sequence Synthetic polypeptide
QVTLKESGPALVKPTQTLTLTCTVSGFSLTAYGVNWVRQPPGKALEWLGMIWGDGNTDYNSALKSRLTISKDNSKSQVVLTMTNMDPVDTATYYCARDRVTATLYAMDYWGQGTLVTVSS

SEQ ID NO: 85 - protein, 120 aa, Artificial Sequence
  Description of Artificial Sequence Synthetic polypeptide
QVQLQESGPGLVKPSGTLSLTCAVSGFSLTAYGVNWVRQPPGKGLEWLGMIWGDGNTDYNSALKSRLTISKDNSKSQVSLKLSSVTAADTAVYYCARDRVTATLYAMDYWGQGTLVTVSS

SEQ ID NO: 86 - protein, 120 aa, Artificial Sequence
  Description of Artificial Sequence Synthetic polypeptide
EVQLVESGGGLVQPGPSLRLSCTVSGFSLTAYGVNWVRQAPGKGLEWLGMIWGDGNTDYNSALKSRLTISKDNSKSIVYLQMNSLKTEDTAVYYCARDRVTATLYAMDYWGQGTLVTVSS

SEQ ID NO: 87 - protein, 120 aa, Artificial Sequence
  Description of Artificial Sequence Synthetic polypeptide
QVQLQQSGPGLVKPSQTLSLTCAVSGFSLTAYGVNWVRQSPSRGLEWLGMIWGDGNTDYNSALKSRLTINKDNSKSQVSLQLNSVTPEDTAVYYCARDRVTATLYAMDYWGQGTLVTVSS

SEQ ID NO: 88 - protein, 107 aa, Artificial Sequence
  Description of Artificial Sequence Synthetic polypeptide
DIQMTQSPSFLSASVGDRVTITCSASQGISNYLNWYQQKPGKAVKLLIYYTSSLHSGVPSRFSGSGSGTEYTLTISSLQPEDFATYYCQQYSKLPRTFGGGTKVEIK

SEQ ID NO: 89 - protein, 107 aa, Artificial Sequence
  Description of Artificial Sequence Synthetic polypeptide
DIQMTQSPSSLSASVGDRVTITCSASQGISNYLNWYQQKPGKAVKLLIYYTSSLHSGVPSRFSGSGSGTDYTLTISSLQPEDFATYYCQQYSKLPRTFGGGTKVEIK

SEQ ID NO: 90 - protein, 107 aa, Artificial Sequence
  Description of Artificial Sequence Synthetic polypeptide
DIQMTQSPSSLSASVGDRVTITCSASQGISNYLNWYQQKPGKVVKLLIYYTSSLHSGVPSRFSGSGSGTDYTLTISSLQPEDVATYYCQQYSKLPRTFGGGTKVEIK

SEQ ID NO: 91 - protein, 107 aa, Artificial Sequence
  Description of Artificial Sequence Synthetic polypeptide
AIRMTQSPFSLSASVGDRVTITCSASQGISNYLNWYQQKPAKAVKLFIYYTSSLHSGVPSRFSGSGSGTDYTLTISSLQPEDFATYYCQQYSKLPRTFGGGTKVEIK

SEQ ID NO: 92 - protein, 107 aa, Artificial Sequence
  Description of Artificial Sequence Synthetic polypeptide
DIQMTQSPSAMSASVGDRVTITCSASQGISNYLNWYQQKPGKVVKRLIYYTSSLHSGVPSRFSGSGSGTEYTLTISSLQPEDFATYYCQQYSKLPRTFGGGTKVEIK

SEQ ID NO: 93 - protein, 107 aa, Artificial Sequence
  Description of Artificial Sequence Synthetic polypeptide
EIVMTQSPPTLSLSPGERVTLSCSASQGISNYLNWYQQKPGQAVKLLIYYTSSLHSGIPARFSGSGSGTDYTLTISSLQPEDFAVYYCQQYSKLPRTFGGGTKVEIK

SEQ ID NO: 94 - protein, 107 aa, Artificial Sequence
  Description of Artificial Sequence Synthetic polypeptide
DIVMTQTPLSLSVTPGQPASISCSASQGISNYLNWYLQKPGQSVKLLIYYTSSLHSGVPDRFSGSGSGTDYTLKISRVEAEDVGVYYCQQYSKLPRTFGGGTKVEIK

SEQ ID NO: 95 - protein, 107 aa, Artificial Sequence
  Description of Artificial Sequence Synthetic polypeptide
DIVMTQSPAFLSVTPGEKVTITCSASQGISNYLNWYQQKPDQAVKLLIYYTSSLHSGVPSRFSGSGSGTDYTFTISSLEAEDAATYYCQQYSKLPRTFGGGTKVEIK

SEQ ID NO: 96 - protein, 5 aa, Artificial Sequence
  Description of Artificial Sequence Synthetic peptide
SAYMH

SEQ ID NO: 97 - protein, 17 aa, Artificial Sequence
  Description of Artificial Sequence Synthetic peptide
RIDPATGKTKYAPKFQA

SEQ ID NO: 98 - protein, 10 aa, Artificial Sequence
  Description of Artificial Sequence Synthetic peptide
SLNWDYGLDY

SEQ ID NO: 99 - protein, 7 aa, Artificial Sequence
  Description of Artificial Sequence Synthetic peptide
GFNIKSA

SEQ ID NO: 100 - protein, 6 aa, Artificial Sequence
  Description of Artificial Sequence Synthetic peptide
DPATGK

SEQ ID NO: 101 - protein, 10 aa, Artificial Sequence
  Description of Artificial Sequence Synthetic peptide
GFNIKSAYMH

SEQ ID NO: 102 - protein, 15 aa, Artificial Sequence
  Description of Artificial Sequence Synthetic peptide
RASKSVSILGTHLIH

SEQ ID NO: 103 - protein, 7 aa, Artificial Sequence
  Description of Artificial Sequence Synthetic peptide
AASNLES

SEQ ID NO: 104 - protein, 9 aa, Artificial Sequence
  Description of Artificial Sequence Synthetic peptide
QQSIEDPWT

SEQ ID NO: 105 - protein, 11 aa, Artificial Sequence
  Description of Artificial Sequence Synthetic peptide
SKSVSILGTHL

SEQ ID NO: 106 - protein, 111 aa, Artificial Sequence
  Description of Artificial Sequence Synthetic polypeptide
DIVLTQSPASLAVSLGQRATISCRASKSVSILGTHLIHWYQQKPGQPPKLLIYAASNLESGVPARFSGSGSETVFTLNIHPVEEEDAATYFCQQSIEDPWTFGGGTKLGIK

SEQ ID NO: 107 - protein, 119 aa, Artificial Sequence
  Description of Artificial Sequence Synthetic polypeptide
EVQLQQSVADLVRPGASLKLSCTASGFNIKSAYMHWVIQRPDQGPECLGRIDPATGKTKYAPKFQAKATITADTSSNTAYLQLSSLTSEDTAIYYCTRSLNWDYGLDYWGQGTSVTVSS

SEQ ID NO: 108 - protein, 119 aa, Artificial Sequence
  Description of Artificial Sequence Synthetic polypeptide
QVQLVQSGAEVKKPGASVKVSCKASGFNIKSAYMHWVRQAPGQGLEWMGRIDPATGKTKYAPKFQARVTMTADTSTNTAYMELSSLRSEDTAVYYCARSLNWDYGLDYWGQGTLVTVSS

SEQ ID NO: 109 - protein, 119 aa, Artificial Sequence
  Description of Artificial Sequence Synthetic polypeptide
QVQLVQSGAEVKKPGASVKVSCKASGFNIKSAYMHWVRQAPGQEPGCMGRIDPATGKTKYAPKFQARVTMTADTSINTAYTELSSLRSEDTATYYCARSLNWDYGLDYWGQGTLVTVSS

SEQ ID NO: 110 - protein, 119 aa, Artificial Sequence
  Description of Artificial Sequence Synthetic polypeptide
QVQLVQSGAEVKKPGSSVKVSCKASGFNIKSAYMHWVRQAPGQGLEWMGRIDPATGKTKYAPKFQARVTITADTSTNTAYMELSSLRSEDTAVYYCARSLNWDYGLDYWGQGTLVTVSS

SEQ ID NO: 111 - protein, 119 aa, Artificial Sequence
  Description of Artificial Sequence Synthetic polypeptide
QVQLVQSGAEVKKPGASVKVSCKASGFNIKSAYMHWVRQAPGQRLEWMGRIDPATGKTKYAPKFQARVTITADTSANTAYMELSSLRSEDTAVYYCARSLNWDYGLDYWGQGTLVTVSS

SEQ ID NO: 112 - protein, 109 aa, Artificial Sequence
  Description of Artificial Sequence Synthetic polypeptide
EIVLTQSPATLSLSPGERATLSCRASKSVSILGTHLIHWYQQKPGQAPRLLIYAASNLESGIPARFSGSGSETDFTLTISSLEPEDFAVYFCQQSIEDPFGGGTKVEIK

SEQ ID NO: 113 - protein, 109 aa, Artificial Sequence
  Description of Artificial Sequence Synthetic polypeptide
EIVLTQSPATLSLSPGERATLSCRASKSVSILGTHLIHWYQQKPGLAPRLLIYAASNLESGIPDRFSGSGSETDFTLTISRLEPEDFAVYFCQQSIEDPFGGGTKVEIK

SEQ ID NO: 114 - protein, 109 aa, Artificial Sequence
  Description of Artificial Sequence Synthetic polypeptide
EIVLTQSPGTLSLSPGERATLSCRASKSVSILGTHLIHWYQQKPGQAPRLLIYAASNLESGIPDRFSGSGSETDFTLTISRLEPEDFAVYFCQQSIEDPFGGGTKVEIK

SEQ ID NO: 115 - protein, 5 aa, Artificial Sequence
  Description of Artificial Sequence Synthetic peptide
DTYMY

SEQ ID NO: 116 - protein, 17 aa, Artificial Sequence
  Description of Artificial Sequence Synthetic peptide
RIDPANGNTKYDPKFQD

SEQ ID NO: 117 - protein, 9 aa, Artificial Sequence
  Description of Artificial Sequence Synthetic peptide
GSYYYAMDY

SEQ ID NO: 118 - protein, 7 aa, Artificial Sequence
  Description of Artificial Sequence Synthetic peptide
GFKTEDT

SEQ ID NO: 119 - protein, 6 aa, Artificial Sequence
  Description of Artificial Sequence Synthetic peptide
DPANGN

SEQ ID NO: 120 - protein, 10 aa, Artificial Sequence
  Description of Artificial Sequence Synthetic peptide
GFKTEDTYMY

SEQ ID NO: 121 - protein, 15 aa, Artificial Sequence
  Description of Artificial Sequence Synthetic peptide
RASESVDSYGNSFMH

SEQ ID NO: 122 - protein, 7 aa, Artificial Sequence
  Description of Artificial Sequence Synthetic peptide
RASNLES

SEQ ID NO: 123 - protein, 9 aa, Artificial Sequence
  Description of Artificial Sequence Synthetic peptide
QQSNEDPYT

SEQ ID NO: 124 - protein, 11 aa, Artificial Sequence
  Description of Artificial Sequence Synthetic peptide
SESVDSYGNSF

SEQ ID NO: 125 - protein, 111 aa, Artificial Sequence
  Description of Artificial Sequence Synthetic polypeptide
DIVLTQSPASLAVSLGQRATISCRASESVDSYGNSFMHWYQQKPGQPPKLLIYRASNLESGIPARFSGSGSRTDFTLTINPVEADDVATYYCQQSNEDPYTFGGGTKLEIK

SEQ ID NO: 126 - protein, 118 aa, Artificial Sequence
  Description of Artificial Sequence Synthetic polypeptide
EVQLQQSGAELVKPGASVKLSCTASGFKTEDTYMYWVKQRPEQGLEWIGRIDPANGNTKYDPKFQDKATITADSSSNTAYLQLSSLPSEDTAVYYCARGSYYYAMDYWGQGTSVTVSS

SEQ ID NO: 127 - protein, 118 aa, Artificial Sequence
  Description of Artificial Sequence Synthetic polypeptide
QVQLVQSGAEVKKPGSSVKVSCKASGFKTEDTYMYWVRQAPGQGLEWIGRIDPANGNTKYDPKFQDRATITADSSTNTAYMELSSLRSEDTAVYYCARGSYYYAMDYWGQGTLVTVSS

SEQ ID NO: 128 - protein, 118 aa, Artificial Sequence
  Description of Artificial Sequence Synthetic polypeptide
QVQLVQSGAEVKKPGASVKVSCKASGFKTEDTYMYWVRQAPGQRLEWIGRIDPANGNTKYDPKFQDRATITADSSANTAYMELSSLRSEDTAVYYCARGSYYYAMDYWGQGTLVTVSS

SEQ ID NO: 129 - protein, 118 aa, Artificial Sequence
  Description of Artificial Sequence Synthetic polypeptide
EVQLVESGGGLVQPGGSLKLSCAASGFKTEDTYMYWVRQASGKGLEWIGRIDPANGNTKYDPKFQDRATISADSSKNTAYLQMNSLKTEDTAVYYCARGSYYYAMDYWGQGTLVTVSS

SEQ ID NO: 130 - protein, 118 aa, Artificial Sequence
  Description of Artificial Sequence Synthetic polypeptide
EVQLVQSGAEVKKPGESLRISCKASGFKTEDTYMYWVRQMPGKGLEWIGRIDPANGNTKYDPKFQDQATISADSSINTAYLQWSSLKASDTAMYYCARGSYYYAMDYWGQGTLVTVSS

SEQ ID NO: 131 - protein, 118 aa, Artificial Sequence
  Description of Artificial Sequence Synthetic polypeptide
QVQLVQSGSELKKPGASVKVSCKASGFKTEDTYMYWVRQAPGQGLEWIGRIDPANGNTKYDPKFQDRAVISADSSVNTAYLQISSLKAEDTAVYYCARGSYYYAMDYWGQGTLVTVSS

SEQ ID NO: 132 - protein, 111 aa, Artificial Sequence
  Description of Artificial Sequence Synthetic polypeptide
DIVLTQSPDSLAVSLGERATINCRASESVDSYGNSFMHWYQQKPGQPPKLLIYRASNLESGVPDRFSGSGSRTDFTLTISSLQAEDVAVYYCQQSNEDPYTFGQGTKLEIK

SEQ ID NO: 133 - protein, 111 aa, Artificial Sequence
  Description of Artificial Sequence Synthetic polypeptide
EIVLTQSPATLSLSPGERATLSCRASESVDSYGNSFMHWYQQKPGQAPKLLIYRASNLESGIPARFSGSGSRTDFTLTISRLEPEDFAVYYCQQSNEDPYTFGQGTKLEIK

SEQ ID NO: 134 - protein, 111 aa, Artificial Sequence
  Description of Artificial Sequence Synthetic polypeptide
DIQLTQSPSSLSASVGDRVTITCRASESVDSYGNSFMHWYQQKPGQAPKLLIYRASNLESGVPSRFSGSGSRTDFTLTISSLQPEDVATYYCQQSNEDPYTFGQGTKLEIK

SEQ ID NO: 135 - protein, 111 aa, Artificial Sequence
  Description of Artificial Sequence Synthetic polypeptide
AIQLTQSPSSLSASVGDRVTITCRASESVDSYGNSFMHWYQQKPGKAPKLLIYRASNLESGVPSRFSGSGSRTDFTLTISSLQPEDFATYYCQQSNEDPYTFGQGTKLEIK

SEQ ID NO: 136 - protein, 111 aa, Artificial Sequence
  Description of Artificial Sequence Synthetic polypeptide
EIVLTQSPDFQSVTPKEKVTITCRASESVDSYGNSFMHWYQQKPDQSPKLLIYRASNLESGVPSRFSGSGSRTDFTLTINSLEAEDAATYYCQQSNEDPYTFGQGTKLEIK

SEQ ID NO: 137 - protein, 5 aa, Artificial Sequence
  Description of Artificial Sequence Synthetic peptide
SYAMS

SEQ ID NO: 138 - protein, 17 aa, Artificial Sequence
  Description of Artificial Sequence Synthetic peptide
HISNGGDYIYYADTVKG

SEQ ID NO: 139 - protein, 12 aa, Artificial Sequence
  Description of Artificial Sequence Synthetic peptide
PSYYSDPWFFDV

SEQ ID NO: 140 - protein, 7 aa, Artificial Sequence
  Description of Artificial Sequence Synthetic peptide
GFTFRSY

SEQ ID NO: 141 - protein, 6 aa, Artificial Sequence
  Description of Artificial Sequence Synthetic peptide
SNGGDY

SEQ ID NO: 142 - protein, 10 aa, Artificial Sequence
  Description of Artificial Sequence Synthetic peptide
GFTFRSYAMS

SEQ ID NO: 143 - protein, 10 aa, Artificial Sequence
  Description of Artificial Sequence Synthetic peptide
SAGSSVSFMH

SEQ ID NO: 144 - protein, 7 aa, Artificial Sequence
  Description of Artificial Sequence Synthetic peptide
DTSKLAS

SEQ ID NO: 145 - protein, 9 aa, Artificial Sequence
  Description of Artificial Sequence Synthetic peptide
LQGSGFPLT

SEQ ID NO: 146 - protein, 6 aa, Artificial Sequence
  Description of Artificial Sequence Synthetic peptide
GSSVSF

SEQ ID NO: 147 - protein, 106 aa, Artificial Sequence
  Description of Artificial Sequence Synthetic polypeptide
ENVLTQSPAIMSASPGEKVTMTCSAGSSVSFMHWYQQKSSTSPKLWIYDTSKLASGVPGRFSGSGSGNSFSLTISSMEAEDVAIYYCLQGSGFPLTFGSGTKLEIK

SEQ ID NO: 148 - protein, 121 aa, Artificial Sequence
  Description of Artificial Sequence Synthetic polypeptide
DVKLVESGEGLVKPGGSLKLSCAASGFTFRSYAMSWVRQTPEKRLEWVAHISNGGDYIYYADTVKGRFTISRDNARNTLYLQMSSLKSEDTAMYYCTRPSYYSDPWFFDVWGTGTTVTVSS

SEQ ID NO: 149 - protein, 121 aa, Artificial Sequence
  Description of Artificial Sequence Synthetic polypeptide
EVQLVESGGGLVQPGGSLRLSCAASGFTFRSYAMSWVRQAPGKGLEWVAHISNGGDYIYYADTVKGRFTISRDNAKNSLYLQMNSLRAEDTAVYYCTRPSYYSDPWFFDVWGQGTTVTVSS

SEQ ID NO: 150 - protein, 121 aa, Artificial Sequence
  Description of Artificial Sequence Synthetic polypeptide
QVQLVESGGGVVQPGRSLRLSCAASGFTFRSYAMSWVRQAPGKGLEWVAHISNGGDYIYYADTVKGRFTISRDNSKNTLYLQMSSLRAEDTAVYYCTRPSYYSDPWFFDVWGQGTTVTVSS

SEQ ID NO: 151 - protein, 121 aa, Artificial Sequence
  Description of Artificial Sequence Synthetic polypeptide
EVQLVESGGGLVQPGGSLRLSCAASGFTFRSYAMSWVRQAPGKGLEWVAHISNGGDYIYYADTVKGRFTISRDNSKNTLYLQMNSLRAEDTAVYYCTRPSYYSDPWFFDVWGQGTTVTVSS

SEQ ID NO: 152 - protein, 121 aa, Artificial Sequence
  Description of Artificial Sequence Synthetic polypeptide
QVQLVQSGSELKKPGASVKVSCKASGFTFRSYAMSWVRQAPGQGLEWVAHISNGGDYIYYADTVKGRFVISRDNSVNTLYLQISSLKAEDTAVYYCTRPSYYSDPWFFDVWGQGTTVTVSS

SEQ ID NO: 153 - protein, 121 aa, Artificial Sequence
  Description of Artificial Sequence Synthetic polypeptide
QVQLVQSGAEVKKPGASVKVSCKASGFTFRSYAMSWVRQAPGQRLEWVAHISNGGDYIYYADTVKGRFTITRDNSANTLYMELSSLRSEDTAVYYCTRPSYYSDPWFFDVWGQGTTVTVSS

SEQ ID NO: 154 - protein, 106 aa, Artificial Sequence
  Description of Artificial Sequence Synthetic polypeptide
ENVLTQSPATLSLSPGERATLSCSAGSSVSFMHWYQQKPGQAPKLLIYDTSKLASGIPARFSGSGSGNDFTLTISSLEPEDFAVYYCLQGSGFPLTFGQGTKLEIK

SEQ ID NO: 155 - protein, 106 aa, Artificial Sequence
  Description of Artificial Sequence Synthetic polypeptide
ENVLTQSPDFQSVTPKEKVTITCSAGSSVSFMHWYQQKPDQSPKLLIYDTSKLASGVPSRFSGSGSGNDFTLTINSLEAEDAATYYCLQGSGFPLTFGQGTKLEIK

SEQ ID NO: 156 - protein, 106 aa, Artificial Sequence
  Description of Artificial Sequence Synthetic polypeptide
DNQLTQSPSSLSASVGDRVTITCSAGSSVSFMHWYQQKPGKVPKLLIYDTSKLASGVPSRFSGSGSGNDFTLTISSLQPEDVATYYCLQGSGFPLTFGQGTKLEIK

SEQ ID NO: 157 - protein, 106 aa, Artificial Sequence
  Description of Artificial Sequence Synthetic polypeptide
ANQLTQSPSSLSASVGDRVTITCSAGSSVSFMHWYQQKPGKAPKLLIYDTSKLASGVPSRFSGSGSGNDFTLTISSLQPEDFATYYCLQGSGFPLTFGQGTKLEIK

SEQ ID NO: 158 - protein, 106 aa, Artificial Sequence
  Description of Artificial Sequence Synthetic polypeptide
DNVLTQSPDSLAVSLGERATINCSAGSSVSFMHWYQQKPGQPPKLLIYDTSKLASGVPDRFSGSGSGNDFTLTISSLQAEDVAVYYCLQGSGFPLTFGQGTKLEIK

SEQ ID NO: 159 - protein, 5 aa, Artificial Sequence
  Description of Artificial Sequence Synthetic peptide
DYNIH

SEQ ID NO: 160 - protein, 17 aa, Artificial Sequence
  Description of Artificial Sequence Synthetic peptide
YINPYNGRTGYNQKFKA

SEQ ID NO: 161 - protein, 9 aa, Artificial Sequence
  Description of Artificial Sequence Synthetic peptide
WDGSSYFDY

SEQ ID NO: 162 - protein, 10 aa, Artificial Sequence
  Description of Artificial Sequence Synthetic peptide
GYTFTDYNIH

SEQ ID NO: 163 - protein, 6 aa, Artificial Sequence
  Description of Artificial Sequence Synthetic peptide
NPYNGR

SEQ ID NO: 164 - protein, 10 aa, Artificial Sequence
  Description of Artificial Sequence Synthetic peptide
SASSSVSYMH

SEQ ID NO: 165 - protein, 7 aa, Artificial Sequence
  Description of Artificial Sequence Synthetic peptide
EISKLAS

SEQ ID NO: 166 - protein, 9 aa, Artificial Sequence
  Description of Artificial Sequence Synthetic peptide
QQWNYPLLT

SEQ ID NO: 167 - protein, 6 aa, Artificial Sequence
  Description of Artificial Sequence Synthetic peptide
SSSVSY

SEQ ID NO: 168 - protein, 106 aa, Artificial Sequence
  Description of Artificial Sequence Synthetic polypeptide
EIVLTQSPAITAASLGQKVTITCSASSSVSYMHWYQQKSGTSPKPWIYEISKLASGVPARFSGSGSGTSYSLTISSMEAEDAAIYYCQQWNYPLLTFGAGTKLELK

SEQ ID NO: 169 - protein, 118 aa, Artificial Sequence
  Description of Artificial Sequence Synthetic polypeptide
EVQLQQSGPVLVKPGASVRMSCKASGYTFTDYNIHWVKQSHGRSLEWVGYINPYNGRTGYNQKFKAKATLTVDKSSSTAYMDLRSLTSEDSAVYYCARWDGSSYFDYWGQGTTLTVSS

SEQ ID NO: 170 - protein, 118 aa, Artificial Sequence
  Description of Artificial Sequence Synthetic polypeptide
QVQLVQSGAEVKKPGSSVKVSCKASGYTFTDYNIHWVRQAPGQGLEWVGYINPYNGRTGYNQKFKARATLTVDKSTSTAYMELSSLRSEDTAVYYCARWDGSSYFDYWGQGTTVTVSS

SEQ ID NO: 171 - protein, 118 aa, Artificial Sequence
  Description of Artificial Sequence Synthetic polypeptide
QVQLVQSGAEVKKPGASVKVSCKASGYTFTDYNIHWVRQAPGQGLEWVGYINPYNGRTGYNQKFKARATLTVDKSTSTAYMELRSLRSDDMAVYYCARWDGSSYFDYWGQGTTVTVSS

SEQ ID NO: 172 - protein, 118 aa, Artificial Sequence
  Description of Artificial Sequence Synthetic polypeptide
QVQLVQSGAEVKKPGASVKVSCKASGYTFTDYNIHWVRQATGQGLEWVGYINPYNGRTGYNQKFKARATLTVNKSISTAYMELSSLRSEDTAVYYCARWDGSSYFDYWGQGTTVTVSS

SEQ ID NO: 173 - protein, 118 aa, Artificial Sequence
  Description of Artificial Sequence Synthetic polypeptide
EVQLVESGGGLVQPGRSLRLSCTASGYTFTDYNIHWVRQAPGKGLEWVGYINPYNGRTGYNQKFKARATLSVDKSKSIAYLQMNSLKTEDTAVYYCARWDGSSYFDYWGQGTTVTVSS

SEQ ID NO: 174 - protein, 118 aa, Artificial Sequence
  Description of Artificial Sequence Synthetic polypeptide
QVQLVQSGSELKKPGASVKVSCKASGYTFTDYNIHWVRQAPGQGLEWVGYINPYNGRTGYNQKFKARAVLSVDKSVSTAYLQISSLKAEDTAVYYCARWDGSSYFDYWGQGTTVTVSS

SEQ ID NO: 175 - protein, 106 aa, Artificial Sequence
  Description of Artificial Sequence Synthetic polypeptide
EIVLTQSPATLSLSPGERATLSCSASSSVSYMHWYQQKPGQAPKLLIYEISKLASGIPARFSGSGSGTDYTLTISSLEPEDFAVYYCQQWNYPLLTFGQGTKLEIK

SEQ ID NO: 176 - protein, 106 aa, Artificial Sequence
  Description of Artificial Sequence Synthetic polypeptide
EIVLTQSPATLSLSPGERATLSCSASSSVSYMHWYQQKPGQAPKLLIYEISKLASGIPARFSGSGSGTDYTLTISRLEPEDFAVYYCQQWNYPLLTFGQGTKLEIK

SEQ ID NO: 177 - protein, 106 aa, Artificial Sequence
  Description of Artificial Sequence Synthetic polypeptide
EIVLTQSPDFQSVTPKEKVTITCSASSSVSYMHWYQQKPDQSPKLLIYEISKLASGVPSRFSGSGSGTDYTLTINSLEAEDAATYYCQQWNYPLLTFGQGTKLEIK

SEQ ID NO: 178 - protein, 106 aa, Artificial Sequence
  Description of Artificial Sequence Synthetic polypeptide
DIQLTQSPSFLSASVGDRVTITCSASSSVSYMHWYQQKPGKAPKLLIYEISKLASGVPSRFSGSGSGTEYTLTISSLQPEDFATYYCQQWNYPLLTFGQGTKLEIK

SEQ ID NO: 179 - protein, 106 aa, Artificial Sequence
  Description of Artificial Sequence Synthetic polypeptide
AIQLTQSPSSLSASVGDRVTITCSASSSVSYMHWYQQKPGKAPKLLIYEISKLASGVPSRFSGSGSGTDYTLTISSLQPEDFATYYCQQWNYPLLTFGQGTKLEIK

SEQ ID NO: 180 - protein, 106 aa, Artificial Sequence
  Description of Artificial Sequence Synthetic polypeptide
AIRLTQSPFSLSASVGDRVTITCSASSSVSYMHWYQQKPAKAPKLFIYEISKLASGVPSRFSGSGSGTDYTLTISSLQPEDFATYYCQQWNYPLLTFGQGTKLEIK

SEQ ID NO: 181 - protein, 106 aa, Artificial Sequence
  Description of Artificial Sequence Synthetic polypeptide
DIVLTQSPDSLAVSLGERATINCSASSSVSYMHWYQQKPGQPPKLLIYEISKLASGVPDRFSGSGSGTDYTLTISSLQAEDVAVYYCQQWNYPLLTFGQGTKLEIK

SEQ ID NO: 182 - protein, 5 aa, Artificial Sequence
  Description of Artificial Sequence Synthetic peptide
GSWMN

SEQ ID NO: 183 - protein, 17 aa, Artificial Sequence
  Description of Artificial Sequence Synthetic peptide
RIYPGDGDTDYSGKFKG

SEQ ID NO: 184 - protein, 12 aa, Artificial Sequence
  Description of Artificial Sequence Synthetic peptide
SGYFNYVPVFDY

SEQ ID NO: 185 - protein, 7 aa, Artificial Sequence
  Description of Artificial Sequence Synthetic peptide
GYTFSGS

SEQ ID NO: 186 - protein, 6 aa, Artificial Sequence
  Description of Artificial Sequence Synthetic peptide
YPGDGD

SEQ ID NO: 187 - protein, 10 aa, Artificial Sequence
  Description of Artificial Sequence Synthetic peptide
GYTFSGSWMN

SEQ ID NO: 188 - protein, 10 aa, Artificial Sequence
  Description of Artificial Sequence Synthetic peptide
SANSTVGYIH

SEQ ID NO: 189 - protein, 7 aa, Artificial Sequence
  Description of Artificial Sequence Synthetic peptide
TTSNLAS

SEQ ID NO: 190 - protein, 8 aa, Artificial Sequence
  Description of Artificial Sequence Synthetic peptide
HQWSFYPT

SEQ ID NO: 191 - protein, 6 aa, Artificial Sequence
  Description of Artificial Sequence Synthetic peptide
NSTVGY

SEQ ID NO: 192 - protein, 105 aa, Artificial Sequence
  Description of Artificial Sequence Synthetic polypeptide
QIVLTQSPAIMSASLGEEIALTCSANSTVGYIHWYQQKSGTSPKLLIYTTSNLASGVPSRFSGSGSGTFYSLTISSVEAEDAADYFCHQWSFYPTFGGGTKLEIK

SEQ ID NO: 193 - protein, 121 aa, Artificial Sequence
  Description of Artificial Sequence Synthetic polypeptide
QIQLQQSGPEVVKPGASVQISCKASGYTFSGSWMNWVKQRPGKGLEWIGRIYPGDGDTDYSGKFKGRATLTADKSSSTAYMRLSSLTSEDSAVYFCARSGYFNYVPVFDYWGQGTTLSVSS

SEQ ID NO: 194 - protein, 121 aa, Artificial Sequence
  Description of Artificial Sequence Synthetic polypeptide
QIQLVQSGAEVKKPGASVKVSCKASGYTFSGSWMNWVRQAPGQGLEWIGRIYPGDGDTDYSGKFKGRATLTADKSTSTAYMELSSLRSEDTAVYYCARSGYFNYVPVFDYWGQGTTVTVSS

SEQ ID NO: 195 - protein, 121 aa, Artificial Sequence
  Description of Artificial Sequence Synthetic polypeptide
QIQLVQSGAEVKKPGSSVKVSCKASGYTFSGSWMNWVRQAPGQGLEWIGRIYPGDGDTDYSGKFKGRATLTADKSTSTAYMELSSLRSEDTAVYYCARSGYFNYVPVFDYWGQGTTVTVSS

SEQ ID NO: 196 - protein, 121 aa, Artificial Sequence
  Description of Artificial Sequence Synthetic polypeptide
EIQLVQSGAEVKKPGESLKISCKASGYTFSGSWMNWVRQMPGKGLEWIGRIYPGDGDTDYSGKFKGQATLSADKSISTAYLQWSSLKASDTAMYYCARSGYFNYVPVFDYWGQGTTVTVSS

SEQ ID NO: 197 - protein, 121 aa, Artificial Sequence
  Description of Artificial Sequence Synthetic polypeptide
QIQLVQSGSELKKPGASVKVSCKASGYTFSGSWMNWVRQAPGQGLEWIGRIYPGDGDTDYSGKFKGRAVLSADKSVSTAYLQISSLKAEDTAVYYCARSGYFNYVPVFDYWGQGTTVTVSS

SEQ ID NO: 198 - protein, 121 aa, Artificial Sequence
  Description of Artificial Sequence Synthetic polypeptide
QIQLVQSGSELKKPGASVKVSCKASGYTFSGSWMNWVRQAPGQGLEWIGRIYPGDGDTDYSGKFKGRAVLSADKSVSMAYLQISSLKAEDTAVYYCARSGYFNYVPVFDYWGQGTTVTVSS

SEQ ID NO: 199 - protein, 121 aa, Artificial Sequence
  Description of Artificial Sequence Synthetic polypeptide
EIQLVESGGGLVQPGRSLRLSCTASGYTFSGSWMNWVRQAPGKGLEWIGRIYPGDGDTDYSGKFKGRATLSADKSKSIAYLQMNSLKTEDTAVYYCARSGYFNYVPVFDYWGQGTTVTVSS

SEQ ID NO: 200 - protein, 105 aa, Artificial Sequence
  Description of Artificial Sequence Synthetic polypeptide
EIVLTQSPATLSLSPGERATLSCSANSTVGYIHWYQQKPGQAPKLLIYTTSNLASGIPARFSGSGSGTDYTLTISSLEPEDFAVYFCHQWSFYPTFGQGTKLEIK

SEQ ID NO: 201 - protein, 105 aa, Artificial Sequence
  Description of Artificial Sequence Synthetic polypeptide
DIQLTQSPSFLSASVGDRVTITCSANSTVGYIHWYQQKPGKAPKLLIYTTSNLASGVPSRFSGSGSGTEYTLTISSLQPEDFATYFCHQWSFYPTFGQGTKLEIK

SEQ ID NO: 202 - protein, 105 aa, Artificial Sequence
  Description of Artificial Sequence Synthetic polypeptide
EIVLTQSPATLSLSPGERATLSCSANSTVGYIHWYQQKPGQAPKLLIYTTSNLASGIPARFSGSGPGTDYTLTISSLEPEDFAVYFCHQWSFYPTFGQGTKLEIK

SEQ ID NO: 203 - protein, 105 aa, Artificial Sequence
  Description of Artificial Sequence Synthetic polypeptide
DIVLTQSPDSLAVSLGERATINCSANSTVGYIHWYQQKPGQPPKLLIYTTSNLASGVPDRFSGSGSGTDYTLTISSLQAEDVAVYFCHQWSFYPTFGQGTKLEIK

SEQ ID NO: 204 - protein, 105 aa, Artificial Sequence
  Description of Artificial Sequence Synthetic polypeptide
EIVLTQSPDFQSVTPKEKVTITCSANSTVGYIHWYQQKPDQSPKLLIYTTSNLASGVPSRFSGSGSGTDYTLTINSLEAEDAATYFCHQWSFYPTFGQGTKLEIK

SEQ ID NO: 205 - protein, 5 aa, Artificial Sequence
  Description of Artificial Sequence Synthetic peptide
SYGMS

SEQ ID NO: 206 - protein, 17 aa, Artificial Sequence
  Description of Artificial Sequence Synthetic peptide
LISSGGSYTYYTDSVKG

SEQ ID NO: 207 - protein, 8 aa, Artificial Sequence
  Description of Artificial Sequence Synthetic peptide
HGGNFFDY

SEQ ID NO: 208 - protein, 7 aa, Artificial Sequence
  Description of Artificial Sequence Synthetic peptide
GFTFRSY

SEQ ID NO: 209 - protein, 6 aa, Artificial Sequence
  Description of Artificial Sequence Synthetic peptide
SSGGSY

SEQ ID NO: 210 - protein, 10 aa, Artificial Sequence
  Description of Artificial Sequence Synthetic peptide
GFTFRSYGMS

SEQ ID NO: 211 - protein, 10 aa, Artificial Sequence
  Description of Artificial Sequence Synthetic peptide
SVSSSVSYMH

SEQ ID NO: 212 - protein, 7 aa, Artificial Sequence
  Description of Artificial Sequence Synthetic peptide
DTSKLAS

SEQ ID NO: 213 - protein, 10 aa, Artificial Sequence
  Description of Artificial Sequence Synthetic peptide
QQWSSNPQYT

SEQ ID NO: 214 - protein, 6 aa, Artificial Sequence
  Description of Artificial Sequence Synthetic peptide
SSSVSY

SEQ ID NO: 215 - protein, 107 aa, Artificial Sequence
  Description of Artificial Sequence Synthetic polypeptide
QIVLTQSPSIMSASPGEKVTMTCSVSSSVSYMHWYQQKSGTSPKRWIYDTSKLASGVPARFSGSGSGTSYSLTISSMEAEDAATYYCQQWSSNPQYTFGGGTKLEIK

SEQ ID NO: 216 - protein, 117 aa, Artificial Sequence
  Description of Artificial Sequence Synthetic polypeptide
EVQLVESGGDLVKPGGSLKLSCAVSGFTFRSYGMSWVRQTPDKRLEWVALISSGGSYTYYTDSVKGRFTISRDNAKNTLYLQMSSLKSEDTAIYYCSRHGGNFFDYWGQGTTLTVSS

SEQ ID NO: 217 - protein, 117 aa, Artificial Sequence
  Description of Artificial Sequence Synthetic polypeptide
EVQLVESGGGLVKPGGSLRLSCAVSGFTFRSYGMSWVRQAPGKGLEWVALISSGGSYTYYTDSVKGRFTISRDNSKNTLYLQMNSLKTEDTAVYYCSRHGGNFFDYWGQGTTVTVSS

SEQ ID NO: 218 - protein, 117 aa, Artificial Sequence
  Description of Artificial Sequence Synthetic polypeptide
EVQLVESGGGLVQPGGSLRLSCAVSGFTFRSYGMSWVRQAPGKGLEWVALISSGGSYTYYTDSVKGRFTISRDNAKNSLYLQMNSLRAEDTAVYYCSRHGGNFFDYWGQGTTVTVSS

SEQ ID NO: 219 - protein, 117 aa, Artificial Sequence
  Description of Artificial Sequence Synthetic polypeptide
QVQLQESGPGLVKPSGTLSLTCAVSGFTFRSYGMSWVRQPPGKGLEWVALISSGGSYTYYTDSVKGRFTISRDNSKNQLSLKLSSVTAADTAVYYCSRHGGNFFDYWGQGTTVTVSS

SEQ ID NO: 220 - protein, 117 aa, Artificial Sequence
  Description of Artificial Sequence Synthetic polypeptide
QVQLVQSGAEVKKPGASVKVSCKVSGFTFRSYGMSWVRQAPGQRLEWVALISSGGSYTYYTDSVKGRFTITRDNSANTLYMELSSLRSEDTAVYYCSRHGGNFFDYWGQGTTVTVSS

SEQ ID NO: 221 - protein, 117 aa, Artificial Sequence
  Description of Artificial Sequence Synthetic polypeptide
EVQLVQSGAEVKKPGESLKISCKVSGFTFRSYGMSWVRQMPGKGLEWVALISSGGSYTYYTDSVKGQFTISRDNPINTLYLQWSSLKASDTAMYYCSRHGGNFFDYWGQGTTVTVSS

SEQ ID NO: 222 - protein, 107 aa, Artificial Sequence
  Description of Artificial Sequence Synthetic polypeptide
AIQLTQSPSSLSASVGDRVTITCSVSSSVSYMHWYQQKPGKAPKLLIYDTSKLASGVPSRFSGSGSGTDYTLTISSLQPEDFATYYCQQWSSNPQYTFGQGTKLEIK

SEQ ID NO: 223 - protein, 107 aa, Artificial Sequence
  Description of Artificial Sequence Synthetic polypeptide
DIQLTQSPSFLSASVGDRVTITCSVSSSVSYMHWYQQKPGKAPKLLIYDTSKLASGVPSRFSGSGSGTEYTLTISSLQPEDFATYYCQQWSSNPQYTFGQGTKLEIK

SEQ ID NO: 224 - protein, 107 aa, Artificial Sequence
  Description of Artificial Sequence Synthetic polypeptide
EIVLTQSPDFQSVTPKEKVTITCSVSSSVSYMHWYQQKPDQSPKLLIYDTSKLASGVPSRFSGSGSGTDYTLTINSLEAEDAATYYCQQWSSNPQYTFGQGTKLEIK

SEQ ID NO: 225 - protein, 107 aa, Artificial Sequence
  Description of Artificial Sequence Synthetic polypeptide
EIVLTQSPATLSLSPGERATLSCSVSSSVSYMHWYQQKPGQAPKLLIYDTSKLASGIPARFSGSGSGTDYTLTISSLEPEDFAVYYCQQWSSNPQYTFGQGTKLEIK

SEQ ID NO: 226 - protein, 107 aa, Artificial Sequence
  Description of Artificial Sequence Synthetic polypeptide
DIVLTQSPLSLPVTPGEPASISCSVSSSVSYMHWYLQKPGQSPKLLIYDTSKLASGVPDRFSGSGSGTDYTLKISRVEAEDVGVYYCQQWSSNPQYTFGQGTKLEIK

SEQ ID NO: 227 - protein, 107 aa, Artificial Sequence
  Description of Artificial Sequence Synthetic polypeptide
EITLTQSPAFMSATPGDKVNISCSVSSSVSYMHWYQQKPGEAPKFIIYDTSKLASGIPPRFSGSGYGTDYTLTINNIESEDAAYYYCQQWSSNPQYTFGQGTKLEIK

SEQ ID NO: 228 - protein, 5 aa, Artificial Sequence
  Description of Artificial Sequence Synthetic peptide
SSWMN

SEQ ID NO: 229 - protein, 17 aa, Artificial Sequence
  Description of Artificial Sequence Synthetic peptide
RIYPGDGDTKYNGKFKG

SEQ ID NO: 230 - protein, 10 aa, Artificial Sequence
  Description of Artificial Sequence Synthetic peptide
RGTGGWYFDV

SEQ ID NO: 231 - protein, 7 aa, Artificial Sequence
  Description of Artificial Sequence Synthetic peptide
GYAFSSS

SEQ ID NO: 232 - protein, 6 aa, Artificial Sequence
  Description of Artificial Sequence Synthetic peptide
YPGDGD

SEQ ID NO: 233 - protein, 10 aa, Artificial Sequence
  Description of Artificial Sequence Synthetic peptide
GYAFSSSWMN

SEQ ID NO: 234 - protein, 15 aa, Artificial Sequence
  Description of Artificial Sequence Synthetic peptide
RASESVDSSGNSFMH

SEQ ID NO: 235 - protein, 7 aa, Artificial Sequence
  Description of Artificial Sequence Synthetic peptide
RASNLES

SEQ ID NO: 236 - protein, 9 aa, Artificial Sequence
  Description of Artificial Sequence Synthetic peptide
QQSFDDPFT

SEQ ID NO: 237 - protein, 11 aa, Artificial Sequence
  Description of Artificial Sequence Synthetic peptide
SESVDSSGNSF

SEQ ID NO: 238 - protein, 111 aa, Artificial Sequence
  Description of Artificial Sequence Synthetic polypeptide
DIVLTQSPASLAVSLGQRATISCRASESVDSSGNSFMHWYQQKPGQPPQLLIYRASNLESGIPARFSGSGSRTDFTLTINPVEADDVATFYCQQSFDDPFTFGSGTKLEIK

SEQ ID NO: 239 - protein, 119 aa, Artificial Sequence
  Description of Artificial Sequence Synthetic polypeptide
QVQLQQSGPELVKPGASVKISCKASGYAFSSSWMNWVKQRPGQGLEWIGRIYPGDGDTKYNGKFKGKATLTADKSSSTAYMHLSSLTSVDSAVYFCARRGTGGWYFDVWGAGTTVTVSS

SEQ ID NO: 240 - protein, 119 aa, Artificial Sequence
  Description of Artificial Sequence Synthetic polypeptide
QVQLVQSGAEVKKPGASVKVSCKASGYAFSSSWMNWVRQAPGQGLEWIGRIYPGDGDTKYNGKFKGRATLTADKSTSTAYMELSSLRSEDTAVYYCARRGTGGWYFDVWGQGTTVTVSS

SEQ ID NO: 241 - protein, 119 aa, Artificial Sequence
  Description of Artificial Sequence Synthetic polypeptide
QVQLVQSGAEVKKPGSSVKVSCKASGYAFSSSWMNWVRQAPGQGLEWIGRIYPGDGDTKYNGKFKGRATLTADKSTSTAYMELSSLRSEDTAVYYCARRGTGGWYFDVWGQGTTVTVSS

SEQ ID NO: 242 - protein, 119 aa, Artificial Sequence
  Description of Artificial Sequence Synthetic polypeptide
EVQLVQSGAEVKKPGESLKISCKASGYAFSSSWMNWVRQMPGKGLEWIGRIYPGDGDTKYNGKFKGQATLSADKSISTAYLQWSSLKASDTAMYYCARRGTGGWYFDVWGQGTTVTVSS

SEQ ID NO: 243 - protein, 119 aa, Artificial Sequence
  Description of Artificial Sequence Synthetic polypeptide
QVQLVQSGSELKKPGASVKVSCKASGYAFSSSWMNWVRQAPGQGLEWIGRIYPGDGDTKYNGKFKGRAVLSADKSVSMAYLQISSLKAEDTAVYYCARRGTGGWYFDVWGQGTTVTVSS

SEQ ID NO: 244 - protein, 111 aa, Artificial Sequence
  Description of Artificial Sequence Synthetic polypeptide
DIVLTQSPDSLAVSLGERATINCRASESVDSSGNSFMHWYQQKPGQPPQLLIYRASNLESGVPDRFSGSGSRTDFTLTISSLQAEDVAVYYCQQSFDDPFTFGQGTKLEIK

SEQ ID NO: 245 - protein, 111 aa, Artificial Sequence
  Description of Artificial Sequence Synthetic polypeptide
EIVLTQSPATLSLSPGERATLSCRASESVDSSGNSFMHWYQQKPGQAPQLLIYRASNLESGIPARFSGSGSRTDFTLTISSLEPEDFAVYYCQQSFDDPFTFGQGTKLEIK

SEQ ID NO: 246 - protein, 111 aa, Artificial Sequence
  Description of Artificial Sequence Synthetic polypeptide
EIVLTQSPATLSLSPGERATLSCRASESVDSSGNSFMHWYQQKPGQAPQLLIYRASNLESGIPARFSGSGSRTDFTLTISRLEPEDFAVYYCQQSFDDPFTFGQGTKLEIK

SEQ ID NO: 247 - protein, 111 aa, Artificial Sequence
  Description of Artificial Sequence Synthetic polypeptide
DIQLTQSPSSLSASVGDRVTITCRASESVDSSGNSFMHWYQQKPGKVPQLLIYRASNLESGVPSRFSGSGSRTDFTLTISSLQPEDVATYYCQQSFDDPFTFGQGTKLEIK

SEQ ID NO: 248 - protein, 111 aa, Artificial Sequence
  Description of Artificial Sequence Synthetic polypeptide
EIVLTQSPDFQSVTPKEKVTITCRASESVDSSGNSFMHWYQQKPDQSPQLLIYRASNLESGVPSRFSGSGSRTDFTLTINSLEAEDAATYYCQQSFDDPFTFGQGTKLEIK

SEQ ID NO: 249 - protein, 111 aa, Artificial Sequence
  Description of Artificial Sequence Synthetic polypeptide
DIVLTQTPLSLPVTPGEPASISCRASESVDSSGNSFMHWYLQKPGQSPQLLIYRASNLESGVPDRFSGSGSRTDFTLKISRVEAEDVGVYYCQQSFDDPFTFGQGTKLEIK

SEQ ID NO: 250 - protein, 5 aa, Artificial Sequence
  Description of Artificial Sequence Synthetic peptide
DYYMY

SEQ ID NO: 251 - protein, 17 aa, Artificial Sequence
  Description of Artificial Sequence Synthetic peptide
TISGGGSYTYSPDSVKG

SEQ ID NO: 252 - protein, 14 aa, Artificial Sequence
  Description of Artificial Sequence Synthetic peptide
ERDIYYGNFNAMVY

SEQ ID NO: 253 - protein, 7 aa, Artificial Sequence
  Description of Artificial Sequence Synthetic peptide
GFTFSDY

SEQ ID NO: 254 - protein, 6 aa, Artificial Sequence
  Description of Artificial Sequence Synthetic peptide
SGGGSY

SEQ ID NO: 255 - protein, 10 aa, Artificial Sequence
  Description of Artificial Sequence Synthetic peptide
GFTFSDYYMY

SEQ ID NO: 256 - protein, 15 aa, Artificial Sequence
  Description of Artificial Sequence Synthetic peptide
RASKSVSTSGYSYMH

SEQ ID NO: 257 - protein, 7 aa, Artificial Sequence
  Description of Artificial Sequence Synthetic peptide
LASNLES

SEQ ID NO: 258 - protein, 9 aa, Artificial Sequence
  Description of Artificial Sequence Synthetic peptide
QHSRDLPWT

SEQ ID NO: 259 - protein, 11 aa, Artificial Sequence
  Description of Artificial Sequence Synthetic peptide
SKSVSTSGYSY

SEQ ID NO: 260 - protein, 111 aa, Artificial Sequence
  Description of Artificial Sequence Synthetic polypeptide
DIVLTQSPVSLTVSLGQRATISCRASKSVSTSGYSYMHWYQQKPGQPPKLLIYLASNLESGVPARFSGSGSGTDFTLNIHPVEEEDAATYYCQHSRDLPWTFGGGTKLEIK

SEQ ID NO: 261 - protein, 123 aa, Artificial Sequence
  Description of Artificial Sequence Synthetic polypeptide
EVQLVESGGGLVKPGGSLKLSCAASGFTFSDYYMYWVRQTPEKRLEWVATISGGGSYTYSPDSVKGRFTISRDNAKNNLYLQMSSLRSEDTAMYFCARERDIYYGNFNAMVYWGRGTSVTVSS

SEQ ID NO: 262 - protein, 123 aa, Artificial Sequence
  Description of Artificial Sequence Synthetic polypeptide
EVQLLESGGGLVQPGGSLRLSCAASGFTFSDYYMYWVRQAPGKGLEWVATISGGGSYTYSPDSVKGRFTISRDNSKNTLYLQMNSLRAEDTAVYYCARERDIYYGNFNAMVYWGRGTLVTVSS

SEQ ID NO: 263 - protein, 123 aa, Artificial Sequence
  Description of Artificial Sequence Synthetic polypeptide
EVQLVESGGGLVQPGGSLRLSCAASGFTFSDYYMYWVRQAPGKGLEWVATISGGGSYTYSPDSVKGRFTISRDNAKNSLYLQMNSLRAEDTAVYYCARERDIYYGNFNAMVYWGRGTLVTVSS

SEQ ID NO: 264 - protein, 123 aa, Artificial Sequence
  Description of Artificial Sequence Synthetic polypeptide
QVQLVESGGGVVQPGRSLRLSCAASGFTFSDYYMYWVRQAPGKGLEWVATISGGGSYTYSPDSVKGRFTISRDNSKNTLYLQMNSLRAEDTAVYYCARERDIYYGNFNAMVYWGRGTLVTVSS

SEQ ID NO: 265 - protein, 123 aa, Artificial Sequence
  Description of Artificial Sequence Synthetic polypeptide
QVQLVESGGGLVKPGGSLRLSCAASGFTFSDYYMYWIRQAPGKGLEWVATISGGGSYTYSPDSVKGRFTISRDNAKNSLYLQMNSLRAEDTAVYYCARERDIYYGNFNAMVYWGRGTLVTVSS

SEQ ID NO: 266 - protein, 111 aa, Artificial Sequence
  Description of Artificial Sequence Synthetic polypeptide
EIVLTQSPGTLSLSPGERATLSCRASKSVSTSGYSYMHWYQQKPGQAPRLLIYLASNLESGIPDRFSGSGSGTDFTLTISRLEPEDFAVYYCQHSRDLPWTFGGGTKVEIK

SEQ ID NO: 267 - protein, 111 aa, Artificial Sequence
  Description of Artificial Sequence Synthetic polypeptide
EIVLTQSPATLSLSPGERATLSCRASKSVSTSGYSYMHWYQQKPGQAPRLLIYLASNLESGIPARFSGSGSGTDFTLTISSLEPEDFAVYYCQHSRDLPWTFGGGTKVEIK

SEQ ID NO: 268 - protein, 111 aa, Artificial Sequence
  Description of Artificial Sequence Synthetic polypeptide
DIQLTQSPSTLSASVGDRVTITCRASKSVSTSGYSYMHWYQQKPGKAPKLLIYLASNLESGVPSRFSGSGSGTEFTLTISSLQPDDFATYYCQHSRDLPWTFGGGTKVEIK

SEQ ID NO: 269 - protein, 111 aa, Artificial Sequence
  Description of Artificial Sequence Synthetic polypeptide
AIQLTQSPSSLSASVGDRVTITCRASKSVSTSGYSYMHWYQQKPGKAPKLLIYLASNLESGVPSRFSGSGSGTDFTLTISSLQPEDFATYYCQHSRDLPWTFGGGTKVEIK

SEQ ID NO: 270 - protein, 5 aa, Artificial Sequence
  Description of Artificial Sequence Synthetic peptide
GYFWN

SEQ ID NO: 271 - protein, 16 aa, Artificial Sequence
  Description of Artificial Sequence Synthetic peptide
YISYDGSNNYNPSLKN

SEQ ID NO: 272 - protein, 11 aa, Artificial Sequence
  Description of Artificial Sequence Synthetic peptide
PSPGTGYAVDY

SEQ ID NO: 273 - protein, 8 aa, Artificial Sequence
  Description of Artificial Sequence Synthetic peptide
GYSITSGY

SEQ ID NO: 274 - protein, 6 aa, Artificial Sequence
  Description of Artificial Sequence Synthetic peptide
SYDGSN

SEQ ID NO: 275 - protein, 11 aa, Artificial Sequence
  Description of Artificial Sequence Synthetic peptide
GYSITSGYFWN

SEQ ID NO: 276 - protein, 16 aa, Artificial Sequence
  Description of Artificial Sequence Synthetic peptide
RSSQSLVHSNGNTYLH

SEQ ID NO: 277 - protein, 7 aa, Artificial Sequence
  Description of Artificial Sequence Synthetic peptide
KVSNRFS

SEQ ID NO: 278 - protein, 9 aa, Artificial Sequence
  Description of Artificial Sequence Synthetic peptide
SQSTHVPFT

SEQ ID NO: 279 - protein, 12 aa, Artificial Sequence
  Description of Artificial Sequence Synthetic peptide
SQSLVHSNGNTY

SEQ ID NO: 280 - protein, 112 aa, Artificial Sequence
  Description of Artificial Sequence Synthetic polypeptide
NVVMTQTPLSLPVSLGDQASISCRSSQSLVHSNGNTYLHWYLQKPGQSPKFLIYKVSNRFSGVPDRFSGGGSGTEFTLKISRVEAEDLGVYFCSQSTHVPFTFGSGTKLEIK

SEQ ID NO: 281 - protein, 120 aa, Artificial Sequence
  Description of Artificial Sequence Synthetic polypeptide
NVQLQESGPGLVKPSQSLSLTCSVAGYSITSGYFWNWIRQFPGNKLEWMGYISYDGSNNYNPSLKNRISITRDTSKNQFFLKLNSVTTEDTATYYCASPSPGTGYAVDYWGQGTSVTVSS

SEQ ID NO: 282 - protein, 120 aa, Artificial Sequence
  Description of Artificial Sequence Synthetic polypeptide
QVQLQESGPGLVKPSETLSLTCTVSGYSITSGYFWNWIRQPPGKGLEWIGYISYDGSNNYNPSLKNRVTISRDTSKNQFSLKLSSVTAADTAVYYCASPSPGTGYAVDYWGQGTLVTVSS

SEQ ID NO: 283 - protein, 120 aa, Artificial Sequence; identical to SEQ ID NO: 282
  Description of Artificial Sequence Synthetic polypeptide

SEQ ID NO: 284 - protein, 120 aa, Artificial Sequence
  Description of Artificial Sequence Synthetic polypeptide
QVQLVESGGGLVQPGGSLRLSCSVSGYSITSGYFWNWVRQAPGKGLEWVGYISYDGSNNYNPSLKNRFTISRDTSKNTFYLQMNSLRAEDTAVYYCASPSPGTGYAVDYWGQGTLVTVSS

SEQ ID NO: 285 - protein, 111 aa, Artificial Sequence
  Description of Artificial Sequence Synthetic polypeptide
VVMTQSPGTLSLSPGERATLSCRSSQSLVHSNGNTYLHWYQQKPGQAPRFLIYKVSNRFSGIPDRFSGSGSGTDFTLTISRLEPEDFAVYFCSQSTHVPFTFGQGTKLEIK

SEQ ID NO: 286 - protein, 112 aa, Artificial Sequence
  Description of Artificial Sequence Synthetic polypeptide
EVVMTQSPATLSLSPGERATLSCRSSQSLVHSNGNTYLHWYQQKPGQAPRFLIYKVSNRFSGIPARFSGSGSGTDFTLTISSLEPEDFAVYFCSQSTHVPFTFGQGTKLEIK

SEQ ID NO: 287 - protein, 112 aa, Artificial Sequence
  Description of Artificial Sequence Synthetic polypeptide
EVVMTQSPATLSVSPGERATLSCRSSQSLVHSNGNTYLHWYQQKPGQAPRFLIYKVSNRFSGIPARFSGSGSGTEFTLTISSLQSEDFAVYFCSQSTHVPFTFGQGTKLEIK

SEQ ID NO: 288 - protein, 112 aa, Artificial Sequence
  Description of Artificial Sequence Synthetic polypeptide
DVQMTQSPSSLSASVGDRVTITCRSSQSLVHSNGNTYLHWYQQKPGKAPKFLIYKVSNRFSGVPSRFSGSGSGTDFTFTISSLQPEDIATYFCSQSTHVPFTFGQGTKLEIK

SEQ ID NO: 289 - protein, 107 aa, Homo sapiens [sequence omitted]

SEQ ID NO: 290 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 291 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 292 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 293 - protein, 115 aa, Artificial Sequence
  Description of Artificial Sequence Synthetic polypeptide
IQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLS

SEQ ID NO: 294 - protein, 23 aa, Artificial Sequence
  Description of Artificial Sequence Synthetic peptide
VIGFRILLLKVAGFNLLMTLRLW

SEQ ID NO: 295 - protein, 2 aa, Artificial Sequence
  Description of Artificial Sequence Synthetic peptide
SS

SEQ ID NO: 296 - protein, 110 aa, Artificial Sequence
  Description of Artificial Sequence Synthetic polypeptide
PEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVT

SEQ ID NO: 297 - protein, 149 aa, Artificial Sequence
  Description of Artificial Sequence Synthetic polypeptide
DLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYE

SEQ ID NO: 298 - protein, 21 aa, Artificial Sequence
  Description of Artificial Sequence Synthetic peptide
ILLGKATLYAVLVSALVLMAM

SEQ ID NO: 299 - protein, 6 aa, Artificial Sequence
  Description of Artificial Sequence Synthetic peptide
VKRKDF

SEQ ID NO: 300 - protein, 137 aa, Artificial Sequence
  Description of Artificial Sequence Synthetic polypeptide
PKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSA

SEQ ID NO: 301 - protein, 144 aa, Artificial Sequence
  Description of Artificial Sequence Synthetic polypeptide
DLKNVFPPKVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSA

SEQ ID NO: 302 - protein, 23 aa, Artificial Sequence
  Description of Artificial Sequence Synthetic peptide
TILYEILLGKATLYAVLVSALVL

SEQ ID NO: 303 - protein, 11 aa, Artificial Sequence
  Description of Artificial Sequence Synthetic peptide
MAMVKRKDSRG

SEQ ID NO: 304 - protein, 5 aa, Artificial Sequence
  Description of Artificial Sequence Synthetic peptide
DGGGS

SEQ ID NO: 305 - protein, 5 aa, Artificial Sequence
  Description of Artificial Sequence Synthetic peptide
TGEKP

SEQ ID NO: 306 - protein, 4 aa, Artificial Sequence
  Description of Artificial Sequence Synthetic peptide
GGRR

SEQ ID NO: 307 - protein, 25 aa, Artificial Sequence
  Description of Artificial Sequence Synthetic peptide
  MISC_FEATURE (1)..(25): This sequence may encompass 1-5 “Gly Gly Gly Gly Ser” repeating units
GGGGSGGGGSGGGGSGGGGSGGGGS

SEQ ID NO: 308 - protein, 14 aa, Artificial Sequence
  Description of Artificial Sequence Synthetic peptide
EGKSSGSGSESKVD

SEQ ID NO: 309 - protein, 18 aa, Artificial Sequence
  Description of Artificial Sequence Synthetic peptide
KESGSVSSEQLAQFRSLD

SEQ ID NO: 310 - protein, 8 aa, Artificial Sequence
  Description of Artificial Sequence Synthetic peptide
GGRRGGGS

SEQ ID NO: 311 - protein, 9 aa, Artificial Sequence
  Description of Artificial Sequence Synthetic peptide
LRQRDGERP

SEQ ID NO: 312 - protein, 12 aa, Artificial Sequence
  Description of Artificial Sequence Synthetic peptide
LRQKDGGGSERP

SEQ ID NO: 313 - protein, 16 aa, Artificial Sequence
  Description of Artificial Sequence Synthetic peptide
LRQKDGGGSGGGSERP

SEQ ID NO: 314 - protein, 5 aa, Artificial Sequence
  Description of Artificial Sequence Synthetic peptide
DYIVH

SEQ ID NO: 315 - protein, 17 aa, Artificial Sequence
  Description of Artificial Sequence Synthetic peptide
WINTYTGTPTYADDFEG

SEQ ID NO: 316 - protein, 13 aa, Artificial Sequence
  Description of Artificial Sequence Synthetic peptide
SWRRGIRGIGFDY

SEQ ID NO: 317 - protein, 7 aa, Artificial Sequence
  Description of Artificial Sequence Synthetic peptide
GYTFTDY

SEQ ID NO: 318 - protein, 6 aa, Artificial Sequence
  Description of Artificial Sequence Synthetic peptide
NTYTGT

SEQ ID NO: 319 - protein, 10 aa, Artificial Sequence
  Description of Artificial Sequence Synthetic peptide
GYTFTDYIVH

SEQ ID NO: 320 - protein, 11 aa, Artificial Sequence
  Description of Artificial Sequence Synthetic peptide
KASKSINKYLA

SEQ ID NO: 321 - protein, 7 aa, Artificial Sequence
  Description of Artificial Sequence Synthetic peptide
DGSTLQS

SEQ ID NO: 322 - protein, 9 aa, Artificial Sequence
  Description of Artificial Sequence Synthetic peptide
QQHNEYPPT

SEQ ID NO: 323 - protein, 7 aa, Artificial Sequence
  Description of Artificial Sequence Synthetic peptide
SKSINKY

SEQ ID NO: 324 - protein, 107 aa, Artificial Sequence
  Description of Artificial Sequence Synthetic polypeptide
DVQMTQSPYNLAASPGESVSINCKASKSINKYLAWYQQKPGKPNKLLIYDGSTLQSGIPSRFSGSGSGTDFTLTIRGLEPEDFGLYYCQQHNEYPPTFGAGTKLELK

SEQ ID NO: 325 - protein, 122 aa, Artificial Sequence
  Description of Artificial Sequence Synthetic polypeptide
QLQLVQSGPELREPGESVKISCKASGYTFTDYIVHWVKQAPGKGLKWMGWINTYTGTPTYADDFEGRFVFSLEASASTANLQISNLKNEDTATYFCARSWRRGIRGIGFDYWGQGVMVTVSS

SEQ ID NO: 326 - protein, 122 aa, Artificial Sequence
  Description of Artificial Sequence Synthetic polypeptide
QLQLVQSGAEVKKPGASVKVSCKASGYTFTDYIVHWVRQAPGQGLEWMGWINTYTGTPTYADDFEGWVTMTLDASISTAYMELSRLRSDDTAVYYCARSWRRGIRGIGFDYWGQGTMVTVSS

SEQ ID NO: 327 - protein, 122 aa, Artificial Sequence
  Description of Artificial Sequence Synthetic polypeptide
QLQLVQSGAEVKKPGASVKVSCKASGYTFTDYIVHWVRQAPGQGLEWMGWINTYTGTPTYADDFEGRVTMTLDASTSTAYMELSSLRSEDTAVYYCARSWRRGIRGIGFDYWGQGTMVTVSS

SEQ ID NO: 328 - protein, 122 aa, Artificial Sequence
  Description of Artificial Sequence Synthetic polypeptide
QLQLVQSGAEVKKPGASVKVSCKASGYTFTDYIVHWVRQAPGQRLEWMGWINTYTGTPTYADDFEGRVTITLDASASTAYMELSSLRSEDMAVYYCARSWRRGIRGIGFDYWGQGTMVTVSS

SEQ ID NO: 329 - protein, 122 aa, Artificial Sequence
  Description of Artificial Sequence Synthetic polypeptide
QLQLVQSGAEVKKPGASVKVSCKASGYTFTDYIVHWVRQATGQGLEWMGWINTYTGTPTYADDFEGRVTMTLNASISTAYMELSSLRSEDTAVYYCARSWRRGIRGIGFDYWGQGTMVTVSS

SEQ ID NO: 330 - protein, 107 aa, Artificial Sequence
  Description of Artificial Sequence Synthetic polypeptide
EVVMTQSPGTLSLSPGERATLSCKASKSINKYLAWYQQKPGQAPRLLIYDGSTLQSGIPDRFSGSGSGTDFTLTISRLEPEDFAVYYCQQHNEYPPTFGQGTKLEIK

SEQ ID NO: 331 - protein, 107 aa, Artificial Sequence
  Description of Artificial Sequence Synthetic polypeptide
EVVMTQSPATLSLSPGERATLSCKASKSINKYLAWYQQKPGQAPRLLIYDGSTLQSGIPARFSGSGSGTDFTLTISSLEPEDFAVYYCQQHNEYPPTFGQGTKLEIK

SEQ ID NO: 332 - protein, 107 aa, Artificial Sequence
  Description of Artificial Sequence Synthetic polypeptide
DVQMTQSPSSLSASVGDRVTITCKASKSINKYLAWYQQKPGKAPKLLIYDGSTLQSGVPSRFSGSGSGTDFTLTISSLQPEDFATYYCQQHNEYPPTFGQGTKLEIK

SEQ ID NO: 333 - protein, 107 aa, Artificial Sequence
  Description of Artificial Sequence Synthetic polypeptide
AVRMTQSPSSFSASTGDRVTITCKASKSINKYLAWYQQKPGKAPKLLIYDGSTLQSGVPSRFSGSGSGTDFTLTISCLQSEDFATYYCQQHNEYPPTFGQGTKLEIK

SEQ ID NO: 334 - protein, 5 aa, Artificial Sequence
  Description of Artificial Sequence Synthetic peptide
NYGVH

SEQ ID NO: 335 - protein, 16 aa, Artificial Sequence
  Description of Artificial Sequence Synthetic peptide
VIWSDGSTDYDTAFIS

SEQ ID NO: 336 - protein, 9 aa, Artificial Sequence
  Description of Artificial Sequence Synthetic peptide
RAVVADFDY

SEQ ID NO: 337 - protein, 6 aa, Artificial Sequence
  Description of Artificial Sequence Synthetic peptide
GFSLTN

SEQ ID NO: 338 - protein, 9 aa, Artificial Sequence
  Description of Artificial Sequence Synthetic peptide
VIWSDGSTD

SEQ ID NO: 339 - protein, 10 aa, Artificial Sequence
  Description of Artificial Sequence Synthetic peptide
GFSLTNYGVH

SEQ ID NO: 340 - protein, 16 aa, Artificial Sequence
  Description of Artificial Sequence Synthetic peptide
KASKEVTIFGSISALH

SEQ ID NO: 341 - protein, 7 aa, Artificial Sequence
  Description of Artificial Sequence Synthetic peptide
NGAKLES

SEQ ID NO: 342 - protein, 9 aa, Artificial Sequence
  Description of Artificial Sequence Synthetic peptide
LQNKEVPFT

SEQ ID NO: 343 - protein, 117 aa, Artificial Sequence
  Description of Artificial Sequence Synthetic polypeptide
QVQLKQSGPGLLQPSQSLSITCTVSGFSLTNYGVHWVRQSPGKGLEWLGVIWSDGSTDYDTAFISRLSISKDNSKSQVFFKLNSLQADDTAIYYCARRAVVADFDYWGQGTTLTVSS

SEQ ID NO: 344 - protein, 119 aa, Artificial Sequence
  Description of Artificial Sequence Synthetic polypeptide
DIVLTQSPASLAVSLGQKATISCKASKEVTIFGSISALHWYQQKPGQPPKLIYNGAKLESGVSARFSDSGSQNRSPFGNQLSFTLTIAPVEADDAATYYCLQNKEVPFTFGSGTKLEIK

SEQ ID NO: 345 - protein, 117 aa, Artificial Sequence
  Description of Artificial Sequence Synthetic polypeptide
QVTLKESGPVLVKPTETLTLTCTVSGFSLTNYGVHWVRQPPGKALEWLGVIWSDGSTDYDTAFISRLTISKDNSKSQVVLTMTNMDPVDTATYYCARRAVVADFDYWGQGTTVTVSS

SEQ ID NO: 346 - protein, 117 aa, Artificial Sequence
  Description of Artificial Sequence Synthetic polypeptide
QVQLQESGPGLVKPSGTLSLTCAVSGFSLTNYGVHWVRQPPGKGLEWLGVIWSDGSTDYDTAFISRLTISKDNSKSQVSLKLSSVTAADTAVYYCARRAVVADFDYWGQGTTVTVSS

SEQ ID NO: 347 - protein, 117 aa, Artificial Sequence
  Description of Artificial Sequence Synthetic polypeptide
QVQLQQSGPGLVKPSQTLSLTCAVSGFSLTNYGVHWVRQSPSRGLEWLGVIWSDGSTDYDTAFISRLTINKDNSKSQVSLQLNSVTPEDTAVYYCARRAVVADFDYWGQGTTVTVSS

SEQ ID NO: 348 - protein, 117 aa, Artificial Sequence
  Description of Artificial Sequence Synthetic polypeptide
EVQLVESGGGLVQPGPSLRLSCTVSGFSLTNYGVHWVRQAPGKGLEWLGVIWSDGSTDYDTAFISRLTISKDNSKSIVYLQMNSLKTEDTAVYYCARRAVVADFDYWGQGTTVTVSS

SEQ ID NO: 349 - protein, 117 aa, Artificial Sequence
  Description of Artificial Sequence Synthetic polypeptide
EVQLVQSGAEVKKPGESLRISCKVSGFSLTNYGVHWVRQMPGKGLEWLGVIWSDGSTDYDTAFISQLTISKDNSISTVYLQWSSLKASDTAMYYCARRAVVADFDYWGQGTTVTVSS

SEQ ID NO: 350 - protein, 117 aa, Artificial Sequence
  Description of Artificial Sequence Synthetic polypeptide
DIVLTQSPDSLAVSLGERATINCKASKEVTIFGSISALHWYQQKPGQPPKLLYNGAKLESGVSARFGVPDRFSRSGSGLDFTLTISSLQAEDVAVYYCLQNKEVPFTFGQGTKLEIK

SEQ ID NO: 351 - protein, 117 aa, Artificial Sequence
  Description of Artificial Sequence Synthetic polypeptide
EIVLTQSPDFQSVTPKEKVTITCKASKEVTIFGSISALHWYQQKPDQSPKLLYNGAKLESGVSARFGVPSRFSRSGSGLDFTLTINSLEAEDAATYYCLQNKEVPFTFGQGTKLEIK

SEQ ID NO: 352 - protein, 117 aa, Artificial Sequence
  Description of Artificial Sequence Synthetic polypeptide
AIQLTQSPSSLSASVGDRVTITCKASKEVTIFGSISALHWYQQKPGKAPKLLYNGAKLESGVSARFGVPSRFSRSGSGLDFTLTISSLQPEDFATYYCLQNKEVPFTFGQGTKLEIK

SEQ ID NO: 353 - protein, 117 aa, Artificial Sequence
  Description of Artificial Sequence Synthetic polypeptide
DIVLTQTPLSLSVTPGQPASISCKASKEVTIFGSISALHWYLQKPGQPPKLLYNGAKLESGVSARFGVPDRFSRSGSGLDFTLKISRVEAEDVGVYYCLQNKEVPFTFGQGTKLEIK

SEQ ID NO: 354 - protein, 5 aa, Artificial Sequence
  Description of Artificial Sequence Synthetic peptide
TFTEN

SEQ ID NO: 355 - protein, 16 aa, Artificial Sequence
  Description of Artificial Sequence Synthetic peptide
IDPEDGTTDYVEKFKN

SEQ ID NO: 356 - protein, 11 aa, Artificial Sequence
  Description of Artificial Sequence Synthetic peptide
GVGSGDYVMDA

SEQ ID NO: 357 - protein, 15 aa, Artificial Sequence
  Description of Artificial Sequence Synthetic peptide
RASQSVSISRHNLIH

SEQ ID NO: 358 - protein, 7 aa, Artificial Sequence
  Description of Artificial Sequence Synthetic peptide
RASNLAS

SEQ ID NO: 359 - protein, 9 aa, Artificial Sequence
  Description of Artificial Sequence Synthetic peptide
QQSGESPRT

SEQ ID NO: 360 - protein, 99 aa, Artificial Sequence
  Description of Artificial Sequence Synthetic polypeptide
LSLGQRATISCRASQSVSISRHNLIHWYQQKPGQQPKLLIYRASNLASGIPARFSGSGSGTDFTLTINPVQADDVATYYCQQSGESPRTFGGGTKLELK

SEQ ID NO: 361 - protein, 108 aa, Artificial Sequence
  Description of Artificial Sequence Synthetic polypeptide
GRGRAQSVVVQASGYTFTENYIYWVKQRPKQGLELIGRIDPEDGTTDYVEKFKNKATLTVDTSSKTAYMQLSSLTSEDTASYFCARGVGSGDYVMDAWGQGASVTVSS

SEQ ID NO: 362 - protein, 10 aa, Artificial Sequence
  Description of Artificial Sequence Synthetic peptide
GYTFTAYYIS

SEQ ID NO: 363 - protein, 16 aa, Artificial Sequence
  Description of Artificial Sequence Synthetic peptide
RIDPEDGSTDYVEKFK

SEQ ID NO: 364 - protein, 9 aa, Artificial Sequence
  Description of Artificial Sequence Synthetic peptide
GNSDYVMDA

SEQ ID NO: 365 - protein, 15 aa, Artificial Sequence
  Description of Artificial Sequence Synthetic peptide
RASQSVSISGINLMH

SEQ ID NO: 366 - protein, 7 aa, Artificial Sequence
  Description of Artificial Sequence Synthetic peptide
RASSLAS

SEQ ID NO: 367 - protein, 9 aa, Artificial Sequence
  Description of Artificial Sequence Synthetic peptide
QQSWESPRT

SEQ ID NO: 368 - protein, 8 aa, Artificial Sequence
  Description of Artificial Sequence Synthetic peptide
GYTFTAYY

SEQ ID NO: 369 - protein, 8 aa, Artificial Sequence
  Description of Artificial Sequence Synthetic peptide
IDPEDGST

SEQ ID NO: 370 - protein, 7 aa, Artificial Sequence
  Description of Artificial Sequence Synthetic peptide
CARGNSD

SEQ ID NO: 371 - protein, 10 aa, Artificial Sequence
  Description of Artificial Sequence Synthetic peptide
QSVSISGINL

SEQ ID NO: 372 - protein, 10 aa, Artificial Sequence
  Description of Artificial Sequence Synthetic peptide
CQQSWESPRT

SEQ ID NO: 373 - protein, 100 aa, Artificial Sequence
  Description of Artificial Sequence Synthetic polypeptide
LCLRQRATISCRASQSVSISGINLMHWYQQRPGQQPKLLIYRASSLASGIPARFSGRGSGTDFTLTIDPVQADDIAAYFCQQSWESPRTFGGGTQLELKR

SEQ ID NO: 374 - protein, 109 aa, Artificial Sequence
  Description of Artificial Sequence Synthetic polypeptide
DWNSRTGLSQVSCKASGYTFTAYYISWVKQRPKQGLELIGRIDPEDGSTDYVEKFKIKATLTADTSSNTAYMQFSSLTFEDTATYFCARGNSDYVMDAWGQGASVTVSS

SEQ ID NO: 375 - protein, 4 aa, Artificial Sequence
  Description of Artificial Sequence Synthetic peptide
  See specification as filed for detailed description of substitutions and preferred embodiments
GGGS

SEQ ID NO: 376 - protein, 20 aa, Artificial Sequence
  Description of Artificial Sequence Synthetic peptide
GGGGSGGGGSGGGGSGGGGS

SEQ ID NO: 377 - protein, 15 aa, Artificial Sequence
  Description of Artificial Sequence Synthetic peptide
GGGGSGGGGSGGGGS

SEQ ID NO: 378 - protein, 4 aa, Artificial Sequence
  Description of Artificial Sequence Synthetic peptide
GGGS

SEQ ID NO: 379 - protein, 95 aa, Homo sapiens
IAGITQAPTSQILAAGRRMTLRCTQDMRHNAMYWYRQDLGLGLRLIHYSNTAGTTGKGEVPDGYSVSRANTDDFPLTLASAVPSQTSVYFCASSD

SEQ ID NO: 380 - protein, 94 aa, Homo sapiens
NAGVTQTPKFHILKTGQSMTLQCAQDMNHGYMSWYRQDPGMGLRLIYYSAAAGTTDKEVPNGYNVSRLNTEDFPLRLVSAAPSQTSVYLCASSY

SEQ ID NO: 381 - protein, 95 aa, Homo sapiens
NAGVTQTPKFRILKIGQSMTLQCTQDMNHNYMYWYRQDPGMGLKLIYYSVGAGITDKGEVPNGYNVSRSTTEDFPLRLELAAPSQTSVYFCASSY

SEQ ID NO: 382 - protein, 95 aa, Homo sapiens
NAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSY

SEQ ID NO: 383 - protein, 95 aa, Homo sapiens
NAGVTQTPKFHILKTGQSMTLQCAQDMNHGYLSWYRQDPGMGLRRIHYSVAAGITDKGEVPDGYNVSRSNTEDFPLRLESAAPSQTSVYFCASSY

SEQ ID NO: 384 - protein, 95 aa, Homo sapiens
NAGVTQTPKFQVLKTGQSMTLQCAQDMNHNSMYWYRQDPGMGLRLIYYSASEGTTDKGEVPNGYNVSRLNKREFSLRLESAAPSQTSVYFCASSE

SEQ ID NO: 385 - protein, 95 aa, Homo sapiens
NAGVTQTPKFRVLKTGQSMTLLCAQDMNHEYMYWYRQDPGMGLRLIHYSVGEGTTAKGEVPDGYNVSRLKKQNFLLGLESAAPSQTSVYFCASSY

SEQ ID NO: 386 - protein, 95 aa, Homo sapiens; identical to SEQ ID NO: 385